(19) United States
(12) Patent Application Publication
(10) Pub. No.: US 2026/0265337 A1
(43) Pub. Date: Sep. 10, 2026

(54) CHIMERIC ANTIGEN RECEPTORS INCLUDING JAK/STAT BINDING DOMAINS AND MODIFIED IMMUNE CELLS

(71) Applicant: Carisma Therapeutics Inc., Philadelphia, PA (US)

(72) Inventors: Nicholas G. Minutolo, Philadelphia, PA (US); Michael Klichinsky, New Hope, PA (US); Kerri Ciccaglione, Collingswood, NJ (US)

(21) Appl. No.: 19/138,964

(22) PCT Filed: Dec. 14, 2023
(86) PCT No.: PCT/US2023/084007

§ 371 Date: Jun. 13, 2025

Related U.S. Application Data
(60) Provisional application No. 63/432,745, filed on Dec. 15, 2022.

(51) Int. Cl. C07K 14/705; A61K 38/00; C07K 14/715; C07K 16/18; C07K 16/40; C12N 5/0786

(52) U.S. Cl. CPC C07K 14/7051; C07K 14/70517; C07K 14/70578; C07K 14/7151; C07K 14/7153; C07K 14/7155; C07K 16/18; C07K 16/40; C12N 5/0645; A61K 38/00; C12N 2510/00

(57) ABSTRACT

The present disclosure pertains to modified immune cells comprising chimeric antigen receptors (CARs) comprising one or more intracellular JAK binding domains and one or more intracellular STAT binding domains and methods of using and making immune cells comprising said CARs.

### CROSS REFERENCE TO RELATED APPLICATIONS

This application claims priority to and the benefit of U.S. Provisional Patent Application No. 63/432,745, filed Dec. 15, 2022, the contents of which are incorporated herein by reference in their entirety.

### BACKGROUND

JAK/STAT proteins are an integral part of cytokine signaling in immune cells. The recruitment of JAKs and STATs to activated cytokine receptors can have a range of various effects, such as induction of cytokine secretion, differentiation, and shift of cell polarization and/or phenotype.

Macrophages are powerful modulators of the immune response and can generally adopt either a pro-inflammatory (M1) or an anti-inflammatory (M2) phenotype. A precise balance of M1/M2 macrophages is important in resolving the body's response to disease and injury, and various diseases include dysregulated M1/M2 phenotypes. For example, macrophages in the tumor microenvironment (TME) are often biased toward an M2 phenotype that safeguards the tumor, while M1 macrophages in atherosclerotic tissue promote plaque progression.

Therefore, a need exists for establish a method to genetically control, modify, and/or maintain the M1/M2 polarization of engineered immune cells for cell therapies and of immune cells in a subject.

### SUMMARY OF THE INVENTION

The present disclosure encompasses, among other things, compositions comprising modified immune cells (e.g., stem cells, macrophages, monocytes, and/or dendritic cells) comprising chimeric antigen receptors (CARs) comprising one or more intracellular JAK binding domains and one or more intracellular STAT binding domains and methods of producing the same.

The present disclosure provides a system wherein the order of one or more intracellular moieties (from cellular membrane proximal to distal) in the CAR architecture directly influences their function.

In one aspect, the present disclosure provides modified immune cells comprising a chimeric antigen receptor (CAR), wherein the CAR comprises: (a) an extracellular domain; (b) a transmembrane domain; and (c) one or more intracellular moieties comprising: (i) a first JAK binding domain or a portion thereof, and (ii) a first STAT binding domain or a portion thereof; wherein a modified immune cell is or comprises a stem cell, macrophage, monocyte, or dendritic cell. In some embodiments, one or more intracellular moieties further comprise: (iii) a first intracellular domain or a portion thereof.

In some embodiments, one or more intracellular moieties comprise, in order (from cellular membrane proximal to distal): (i) a first JAK binding domain or portion thereof, a first STAT binding domain or portion thereof, and a first intracellular domain or portion thereof; (ii) a first STAT binding domain or portion thereof, a first JAK binding domain or portion thereof, and a first intracellular domain or portion thereof; (iii) a first JAK binding domain or portion thereof, a first intracellular domain or portion thereof, and a first STAT binding domain or portion thereof; (iv) a first STAT binding domain or portion thereof, a first intracellular domain or portion thereof, and a first JAK binding domain or portion thereof; (v) a first intracellular domain or portion thereof, a first JAK binding domain or portion thereof, and a first STAT binding domain or portion thereof; (vi) a first intracellular domain or portion thereof, a first STAT binding domain or portion thereof, and a first JAK binding domain or portion thereof; (vii) a first JAK binding domain or portion thereof and a first STAT binding domain or portion thereof; or (viii) a first STAT binding domain or portion thereof and a first JAK binding domain or portion thereof.

In some embodiments, one or more intracellular moieties further comprise: (iv) a second JAK binding domain or a portion thereof, (v) a second STAT binding domain or a portion thereof, (vi) a second intracellular domain or a portion thereof, or combinations thereof.

In some embodiments, a first JAK binding domain or portion thereof is derived from a first cytokine receptor. In some embodiments, a first STAT binding domain or portion thereof is derived from a second cytokine receptor. In some embodiments, a second JAK binding domain or portion thereof is derived from a third cytokine receptor. In some embodiments, a second STAT binding domain or portion thereof is derived from a fourth cytokine receptor. In some embodiments, a first cytokine receptor and a second cytokine receptor are different cytokine receptors. In some embodiments, a first cytokine receptor and a second cytokine receptor are the same cytokine receptor. In some embodiments, a third cytokine receptor and a fourth cytokine receptor are different cytokine receptors. In some embodiments, a third cytokine receptor and a fourth cytokine receptor are the same cytokine receptor. In some embodiments, a first cytokine receptor and a third cytokine receptor are different cytokine receptors. In some embodiments, a first cytokine receptor and a third cytokine receptor are the same cytokine receptor. In some embodiments, a second cytokine receptor and a fourth cytokine receptor are different cytokine receptors. In some embodiments, a second cytokine receptor and a fourth cytokine receptor are the same cytokine receptor.

In some embodiments, a first cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3. In some embodiments, a second cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3. In some embodiments, a third cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL 7Ra, IL9Ra, IL 10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3. In some embodiments, a fourth cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a first JAK binding domain or portion thereof binds JAK1, JAK2, JAK3, TYK2, or combinations thereof. In some embodiments, a second JAK binding domain or portion thereof binds JAK1, JAK2, JAK3, TYK2, or combinations thereof.

In some embodiments, a first STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof. In some embodiments, a second STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof.

In some embodiments, a first intracellular domain or portion thereof is or comprises a CD34, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL12R, IL13R, IL 14R, IL15R, IL17R, IFNaR, IFNgR, TNFR, CSFIR, CSF2R, Dap10, CD36, Dectin-1, ICOSL, or Syk intracellular domain, a portion of any of the foregoing, or combinations thereof. In some embodiments, a second intracellular domain or portion thereof is or comprises a CD3ζ, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL12R, IL13R, IL 14R, IL15R, IL 17R, IFNaR, IFNgR, TNFR, CSFIR, CSF2R, Dap10, CD36, Dectin-1, ICOSL, or Syk intracellular domain, a portion of any of the foregoing, or combinations thereof. In some embodiments, a first intracellular domain comprise a CD3 intracellular domain or an FcRγ intracellular domain. In some embodiments, a second intracellular domain comprise a CD35 intracellular domain or an FcRγ intracellular domain.

In some embodiments, an extracellular domain is or comprises an scFv, VHH antibody, centyrin, darpin, or nanobody.

In some embodiments, a transmembrane domain is or comprises a CD8a, CD28, CD40, MyD88 CD64, CD32a, CD32c, CD16a, CD3zeta, ICOS, Dectin-1, DNGR1, SLAMF7, TRL1, TLR2, TLR3, TRL4, TLR5, TLR6, TLR7, TLR8, or TLR9 transmembrane domain.

In some embodiments, a CAR further comprises an extracellular leader domain. In some embodiments, an extracellular leader domain comprises a CD8a extracellular leader domain.

In some embodiments, a CAR further comprises an extracellular hinge domain. In some embodiments, an extracellular hinge domain comprises: a CD8a extracellular hinge domain, a CD28 extracellular hinge domain, a DNGR-1 extracellular hinge domain, a Dectin-1 extracellular hinge domain, or an IgG4 extracellular hinge domain.

In some embodiments, a CAR has or comprises: (a) an amino acid sequence selected from Table 2; (b) an amino acid sequence that differs from a sequence selected from Table 2 by no more than five substitutions, additions, or deletions; or (c) an amino acid sequence that is at least 80% identical to a sequence selected from Table 2.

In another aspect, the present disclosure provides pharmaceutical compositions comprising a modified immune cell as described herein. In some embodiments, a pharmaceutical composition comprises a pharmaceutically acceptable carrier.

In another aspect, the present disclosure provides chimeric antigen receptors (CARs) comprising: (a) an extracellular domain; (b) a transmembrane domain; and (c) one or more intracellular moieties comprising: (i) a first JAK binding domain or a portion thereof, and (ii) a first STAT binding domain or a portion thereof. In some embodiments, one or more intracellular moieties further comprise: (iii) a first intracellular domain or a portion thereof.

In some embodiments, one or more intracellular moieties are in order (from cellular membrane proximal to distal): (i) a first JAK binding domain or portion thereof, a first STAT binding domain or portion thereof, and a first intracellular domain or portion thereof; (ii) a first STAT binding domain or portion thereof, a first JAK binding domain or portion thereof, and a first intracellular domain or portion thereof; (iii) a first JAK binding domain or portion thereof, a first intracellular domain or portion thereof, and a first STAT binding domain or portion thereof; (iv) a first STAT binding domain or portion thereof, a first intracellular domain or portion thereof, and a first JAK binding domain or portion thereof; (v) a first intracellular domain or portion thereof, a first JAK binding domain or portion thereof, and a first STAT binding domain or portion thereof; (vi) a first intracellular domain or portion thereof, a first STAT binding domain or portion thereof, and a first JAK binding domain or portion thereof; (vii) a first JAK binding domain or portion thereof and a first STAT binding domain or portion thereof; or (viii) a first STAT binding domain or portion thereof and a first JAK binding domain or portion thereof.

In some embodiments, one or more intracellular moieties further comprise: (iv) a second JAK binding domain or a portion thereof, (v) a second STAT binding domain or a portion thereof, (vi) a second intraceullular domain or a portion thereof, or combinations thereof.

In some embodiments, a first JAK binding domain or portion thereof is derived from a first cytokine receptor. In some embodiments, a first STAT binding domain or portion thereof is derived from a second cytokine receptor. In some embodiments, a second JAK binding domain or portion thereof is derived from a third cytokine receptor. In some embodiments, a second STAT binding domain or portion thereof is derived from a fourth cytokine receptor. In some embodiments, a first cytokine receptor and a second cytokine receptor are different cytokine receptors. In some embodiments, a first cytokine receptor and a second cytokine receptor are the same cytokine receptor. In some embodiments, a third cytokine receptor and a fourth cytokine receptor are different cytokine receptors. In some embodiments, a third cytokine receptor and a fourth cytokine receptor are the same cytokine receptor. In some embodiments, a first cytokine receptor and a third cytokine receptor are different cytokine receptors. In some embodiments, a first cytokine receptor and a third cytokine receptor are the same cytokine receptor. In some embodiments, a second cytokine receptor and a fourth cytokine receptor are different cytokine receptors. In some embodiments, a second cytokine receptor and a fourth cytokine receptor are the same cytokine receptor.

In some embodiments, a first cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR¹, TNFR2, TREM1, TREM2, or Tyro3. In some embodiments, a second cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3. In some embodiments, a third cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL 10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3. In some embodiments, a fourth cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a first JAK binding domain or portion thereof binds JAK1, JAK2, JAK3, TYK2, or combinations thereof. In some embodiments, a second JAK binding domain or portion thereof binds JAK1, JAK2, JAK3, TYK2, or combinations thereof.

In some embodiments, a STAT binding domain or portion thereof binds STAT1, STAT2, STAT3, STAT4, STAT5A, STAT5B, STAT6, a minimal STAT1 binding motif, a minimal STAT3 binding motif, a minimal STAT5a binding motif, a minimal STAT6 binding motif, or combinations thereof. In some embodiments, a second STAT binding domain or portion thereof binds STAT1, STAT2, STAT3, STAT4, STATSA, STAT5B, STAT6, a minimal STAT1 binding motif, a minimal STAT3 binding motif, a minimal STAT5A binding motif, a minimal STAT6 binding motif, or combinations thereof.

In some embodiments, a first intracellular domain or a portion thereof is or comprises a CD34, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL12R, IL13R, IL14R, IL15R, IL17R, IFNaR, IFNgR, TNFR, CSFIR, CSF2R, Dap10, CD36, Dectin-1, ICOSL, or Syk intracellular domain, a portion of any of the foregoing, or combinations thereof. In some embodiments, a second intracellular domain or portion thereof is or comprises a CD34, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL12R, IL13R, IL 14R, IL15R, IL17R, IFNaR, IFNgR, TNFR, CSFIR, CSF2R, Dap10, CD36, Dectin-1, ICOSL, or Syk intracellular domain, a portion of any of the foregoing, or combinations thereof.

In some embodiments, a first intracellular domain comprises a CD3ζ intracellular domain or an FcRγ intracellular domain. In some embodiments, a second intracellular domain comprises a CD32 intracellular domain or an FcRγ intracellular domain.

In some embodiments, an extracellular domain is or comprises an scFv, VHH antibody, centyrin, darpin, or nanobody.

In some embodiments, a transmembrane domain is or comprises a CD8a, CD28, CD40, MyD88 CD64, CD32a, CD32c, CD16a, CD3zeta, ICOS, Dectin-1, DNGR1, SLAMF7, TRL1, TLR2, TLR3, TRL4, TLR5, TLR6, TLR7, TLR8, or TLR9 transmembrane domain.

In some embodiments, a CAR further comprises an extracellular leader domain. In some embodiments, an extracellular leader domain comprises a CD8a extracellular leader domain.

In some embodiments, a CAR further comprises an extracellular hinge domain. In some embodiments, an extracellular hinge domain comprises: a CD8a extracellular hinge domain, a CD28 extracellular hinge domain, a DNGR-1 extracellular hinge domain, a Dectin-1 extracellular hinge domain, or an IgG4 extracellular hinge domain.

In some embodiments, a CAR has or comprises: (a) an amino acid sequence selected from Table 2; (b) an amino acid sequence that differs from a sequence selected from Table 2 by no more than five substitutions, additions, or deletions; or (c) an amino acid sequence that is at least 80% identical to a sequence selected from Table 2.

In another aspect, the present disclosure provides nucleic acid constructs comprising one or more nucleic acid sequences encoding: (a) an extracellular domain; (b) a transmembrane domain; and (c) one or more intracellular moieties comprising: (i) a first JAK binding domain or a portion thereof, and (ii) a first STAT binding domain or a portion thereof; wherein the nucleic acid construct encodes a chimeric antigen receptor (CAR) comprising (a) through (c). In some embodiments, one or more intracellular moieties further comprise: (iii) a first intracellular domain or a portion thereof.

In some embodiments, one or more intracellular moieties further comprise: (iv) a second JAK binding domain or a portion thereof, (v) a second STAT binding domain or a portion thereof, (vi) a second intracellular domain or a portion thereof, or combinations thereof.

In some embodiments, a nucleic acid construct further comprises one or more nucleic acid sequences encoding: (d) one or more extracellular leader domains, (e) one or more extracellular hinge domains, (f) one or more cleavage peptides, or combinations thereof. In some embodiments, cleavage peptide is or comprises a P2A, F2A, E2A or T2A peptide.

In some embodiments, a nucleic acid construct has or comprises: (a) a nucleotide sequence selected from Table 3; (b) a nucleotide sequence that differs from a sequence selected from Table 3 by no more than five substitutions, additions, or deletions; or (c) a nucleotide acid sequence that is at least 80% identical to a sequence selected from Table 3.

In some embodiments, a nucleic acid construct comprises DNA or messenger RNA (mRNA).

In some embodiments, a nucleic acid construct comprises a modification selected from: a modified nucleotide, an alteration to the 5′ untranslated region (UTR), an alteration to the 3′ UTR, a cap structure, a poly(A) tail, or combinations thereof. In some embodiments, a cap structure comprises AGCap1, m6AGCap1, or Anti-Reverse Cap Analog (ARCA). In some embodiments, a modified nucleotide comprises pseudouridine (PsU), 5-methoxyuridine (5moU), 5-methylcytidine/pseudouridine (5meC PsU), N1-methyl-pseudouridine (NlmPsU), or combinations thereof.

In some embodiments, a nucleic acid construct is a purified nucleic acid construct.

In some embodiments, a nucleic acid construct is codon-optimized. In some embodiments, a nucleic acid construct is codon-optimized for expression in a stem cell, monocyte, macrophage, or dendritic cell.

In another aspect, the present disclosure provides pharmaceutical compositions comprising a nucleic acid construct as described herein. In some embodiments, a pharmaceutical composition comprises a pharmaceutically acceptable carrier.

In another aspect, the present disclosure provides methods of treating a disease or disorder in a subject, comprising: administering to the subject a therapeutically effective amount of the pharmaceutical composition as described herein, wherein at least one sign or symptom of the disease or disorder is improved in the subject after administration.

In some embodiments, a step of administering is or comprises transarterial, subcutaneous, intravenous, intradermal, intratumoral, intranodal, intramedullary, intramuscular, or intraperitoneal delivery.

In another aspect, the present disclosure provides methods of modifying an immune cell, comprising: delivering to the immune cell a nucleic acid construct as described herein, thereby producing a modified immune cell, wherein the modified immune cell is or comprises a macrophage, monocyte, dendritic cell, or stem cell.

In some embodiments, a nucleic acid construct comprises DNA or messenger RNA (mRNA).

In some embodiments, a nucleic acid construct comprises a modification selected from: a modified nucleotide, an alteration to the 5′ untranslated region (UTR), an alteration to the 3′ UTR, a cap structure, a poly(A) tail, or combinations thereof. In some embodiments, a cap structure comprises AGCap1, m6AGCap1, or Anti-Reverse Cap Analog (ARCA). In some embodiments, a modified nucleotide comprises pseudouridine (PsU), 5-methoxyuridine (5moU), 5-methylcytidine/pseudouridine (5meC PsU), N1-methyl-pseudouridine (NlmPsU), or combinations thereof.

In some embodiments, a nucleic acid construct is a purified nucleic acid construct. In some embodiments, a purified nucleic acid construct is produced by a method comprising silica membrane purification, high performance liquid chromatography (HPLC), Dynabeads, LiCl precipitation, phenol-chloroform extraction, resin based purification, polyA isolation, RNeasy, or combinations thereof.

In some embodiments, a nucleic acid construct is codon-optimized. In some embodiments, a nucleic acid construct is codon-optimized for expression in a stem cell, monocyte, macrophage, or dendritic cell.

In some embodiments, delivering comprises electroporation or transfection with the nucleic acid construct. In some embodiments, a nucleic acid construct is encapsulated within a delivery vehicle. In some embodiments, a delivery vehicle is or comprises a liposome, a lipid nanoparticle, a polymer, an adeno-associated viral (AAV) vector, an adenoviral vector, a retroviral vector or combinations thereof. In some embodiments, a liposome or lipid nanoparticle comprises one or more cationic lipids, one or more non-cationic lipids, one or more cholesterol-based lipids, one or more PEG-modified lipids, or combinations thereof. In some embodiments, a retroviral vector comprises a lentiviral vector or a gammaretroviral vector. In some embodiments, a lentiviral vector is packaged with a Vpx protein. In some embodiments, an adenoviral vector comprises an Ad2 vector or an Ad5 vector. In some embodiments, an Ad5 vector comprises an Ad5f35 adenoviral vector.

In some embodiments, a method of the present disclosure further comprises delivering to the immune cell an additional payload. In some embodiments, an additional payload is or comprises a pathogen recognition receptor agonist, polyinosinic: polycytidylic acid (poly I:C), a TLR7/8 agonist, a CpG oligodeoxynucleotide, a NOD-like receptor (NLR) agonist, a RIG-I-like receptor (RLR) agonist, a C-type lectins receptor (CLR) agonist, a cytosolic DNA sensing, the cyclic GMP-AMP synthase-stimulator of interferon genes (cGAS-STING) agonist, an interferon-inducible protein 16 (IFI16) agonist, a DEAD-box helicase 41 (DDX41) agonist, an LRR binding FLII interacting protein 1 (LRRFIP1) agonist, an absent in melanoma 2 (AIM2) agonist, an aryl hydrocarbon receptor (AhR) ligand, or combinations thereof. In some embodiments, a nucleic acid construct and an additional payload are encapsulated within a delivery vehicle.

### BRIEF DESCRIPTION OF THE DRAWING

The drawings are for illustration purposes only, not for limitation.

FIG. 1 shows exemplary designs for CAR constructs comprising JAK, STAT, and ITAM domains.

FIG. 2 shows exemplary functions of CAR constructs comprising JAK and STAT domains in cells (e.g., myeloid cells, e.g., macrophages, monocytes, etc.).

FIG. 3 shows exemplary cytokine secretion in macrophages expressing anti-HER2 CAR constructs comprising JAK/STAT domains distal from plasma membrane when incubated with PBS, HER2, or Mesothelin for 24 hours. IP-10 and TNFα cytokines were assessed.

FIG. 4 shows exemplary cytokine secretion in macrophages expressing anti-HER2 CAR constructs comprising JAK/STAT domains proximal to plasma membrane when incubated with PBS, HER2, or Mesothelin for 24 hours. IP-10 and TNFα cytokines were assessed.

FIG. 5 shows exemplary phosphorylation of STAT1 in macrophages expressing constitutive, inducible, or mutated anti-HER2 CAR constructs relative to control cells when incubated with PBS or HER2 for 1 or 2 hours, as assayed by Western Blot.

FIG. 6 shows exemplary viability and recovery percentages of macrophages expressing constitutive, inducible, or mutated anti-HER2 CAR constructs relative to control cells.

FIG. 7A and FIG. 7B show exemplary CAR expression via histogram (FIG. 7A) and quantification by CAR+ percentage and mean fluorescence intensity (FIG. 7B) in macrophages expressing constitutive, inducible, or mutated anti-HER2 CAR constructs relative to control cells, as measured by flow cytometry.

FIG. 8 shows exemplary expression of M1 markers (CD80, CD86) and M2 markers (CD163, CD206) via quantification by mean fluorescence intensity in macrophages expressing constitutive, inducible, or mutated anti-HER2 CAR constructs relative to control cells, as measured by flow cytometry.

FIG. 9 shows exemplary expression of M1 marker (CD86) and M2 markers (CD163, CD206) via histogram in macrophages expressing a constitutive anti-HER2 CAR construct relative to control cells, as measured by flow cytometry.

FIG. 10 shows exemplary tumor cell death when co-cultured at different ratios for 72 hours with macrophages expressing constitutive, inducible, or mutated anti-HER2 CAR constructs relative to control cells, as measured by GFP intensity from tumor cell expression. Ratios of 1:2 E:T (20,000 macrophages: 40,000 Au565 cells) and 2:1 E:T (20,000 macrophages: 10,000 Au565 cells) were assessed.

FIG. 11A and FIG. 11B show exemplary tumor cell death in wild-type (Wt) and HER2+Panc1 cells (FIG. 11A) and MDA-MB-468 cells (FIG. 11B) when co-cultured at 3:1 E:T ratio for 48 hours with macrophages expressing constitutive, inducible, or mutated anti-HER2 CAR constructs relative to control cells, as measured by GFP intensity from tumor cell expression.

FIG. 12 shows exemplary designs for CAR constructs comprising an ITAM domain, a JAK domain, and one or more STAT domains.

FIG. 13 shows exemplary activity via STAT1 pathway activation in a HEEK cell reporter line.

FIG. 14A and FIG. 14B show exemplary CAR expression via histogram (FIG. 14A) and quantification by CAR+ percentage and mean fluorescence intensity (FIG. 14B) in macrophages expressing inducible, multi-STAT anti-HER2 CAR constructs relative to control cells, as measured by flow cytometry.

FIG. 15 shows exemplary tumor cell death when co-cultured at different ratios for 72 hours with macrophages expressing inducible, multi-STAT anti-HER2 CAR constructs relative to control cells, as measured by GFP intensity from tumor cell expression. Ratios of 1:1 E:T (20,000 macrophages: 20,000 Au565 cells) and 1:4 E:T (20,000 macrophages: 80,000 Au565 cells) were assessed.

FIG. 16 shows antigen-specific and STAT1-specific increased PD-L1 expression in macrophages comprising inducible, multi-STAT anti-HER2 CAR constructs.

FIG. 17 shows an exemplary principal component analysis plot of broad panel cytokine data.

FIG. 18 shows exemplary data for a single cytokine (TNFα) taken from the broad panel cytokine analysis.

FIG. 19A, FIG. 19B, and FIG. 19C show exemplary data for IFNγ-associated cytokines (FIG. 19A-CXCL10 (IP-10); FIG. 19B-CXCL11 (IP-9); and FIG. 19C-CXCL9 (MIG)) taken from the broad panel cytokine analysis.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Activation: As used herein, the term “activation” refers to the state of a cell, for example a monocyte, macrophage, or dendritic cell that has been sufficiently stimulated to induce detectable cellular proliferation or has been stimulated to exert its effector function. Activation can also be associated with induced cytokine production, phagocytosis, cell signaling, target cell killing, and/or antigen processing and presentation.

Activated monocytes/macrophages/dendritic cells: As used herein, the term “activated monocytes/macrophages/dendritic cells” refers to, among other things, monocyte/macrophage/dendritic cells that are undergoing cell division or exerting effector function. The term “activated monocytes/macrophages/dendritic cells” refers to, among others thing, cells that are performing an effector function or exerting any activity not seen in the resting state, including phagocytosis, cytokine secretion, proliferation, gene expression changes, metabolic changes, and other functions.

Agent: As used herein, the term “agent” (or “biological agent” or “therapeutic agent”), refers to a molecule that may be expressed, released, secreted or delivered to a target by a modified cell described herein. An agent includes, but is not limited to, a nucleic acid, an antibiotic, an anti-inflammatory agent, an antibody or fragments thereof, an antibody agent or fragments thereof, a growth factor, a cytokine, an enzyme, a protein (e.g., an RNAse inhibitor), a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′) 2 fragments, Fd′ fragments, Fd fragments, and isolated CDRs or sets thereof, single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, in some embodiments, an antibody agent utilized in accordance with the present invention is in a format selected from, but not limited to, intact IgA, IgG, IgE or IgM antibodies; bi- or multi-specific antibodies (e.g., Zybodies®, etc); antibody fragments such as Fab fragments, Fab′ fragments, F(ab′) 2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies, Adnectins®; Affilins®; Trans-bodies®; Affibodies®; TrimerX®; MicroProteins; Fynomers®, Centyrins®; and KALBITOR®s. In some embodiments, an antibody agent may lack a covalent modification (e.g., attachment of a glycan) that it would have if produced naturally. In some embodiments, an antibody agent may contain a covalent modification (e.g., attachment of a glycan, a payload [e.g., a detectable moiety, a therapeutic moiety, a catalytic moiety, etc], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain. In some embodiments, an antibody agent is not and/or does not comprise a polypeptide whose amino acid sequence includes structural elements recognized by those skilled in the art as an immunoglobulin variable domain. In some embodiments, an antibody agent may be or comprise a molecule or composition which does not include immunoglobulin structural elements (e.g., a receptor or other naturally occurring molecule which includes at least one antigen binding domain).

Antibody fragment: As used herein, the term “antibody fragment” refers to a portion of an intact antibody and refers to the antigenic determining variable regions of an intact antibody. Examples of antibody fragments include, but are not limited to, Fab, Fab′, F(ab′) 2, and Fv fragments, linear antibodies, scFv antibodies, and multispecific antibodies formed from antibody fragments and human and humanized versions thereof.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Synthetic antibody: As used herein, the term “synthetic antibody” refers to an antibody that is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Anti-tumor effect: As used herein, the term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the invention in prevention of the occurrence of a tumor in the first place.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Xenogenic: As used herein, the term “xenogeneic” refers to any material (e.g., a population of cells) derived from an animal of a different species.

Cancer: As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand: As used herein, the term “co-stimulatory ligand” refers to a molecule on an antigen presenting cell (e.g., an APC, dendritic cell, B cell, and the like) that specifically binds a cognate co-stimulatory molecule on a monocyte/macrophage/dendritic cell, thereby providing a signal which mediates a monocyte/macrophage/dendritic cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a monocyte/macrophage/dendritic cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In one embodiment, cytotoxicity of the metabolically enhanced cells is improved, e.g. increased cytolytic activity of macrophages.

Effective amount: As used herein, “effective amount” and “therapeutically effective amount” are interchangeable, and refer to an amount of a compound, formulation, material, or composition, as described herein effective to achieve a particular biological result or provides a manufacturing, therapeutic or prophylactic benefit. Such results may include, but are not limited to, anti-tumor activity as determined by any means suitable in the art.

Effector function: As used herein, “effector function” or “effector activity” refers to a specific activity carried out by an immune cell in response to stimulation of the immune cell. For example, an effector function of macrophages to engulf and digest cellular debris, foreign substances, microbes, cancer cells and other unhealthy cells by phagocytosis.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., IRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, monocytes, macrophages, and/or dendritic cells. In one embodiment, monocytes, macrophages, or dendritic cells that are expanded ex vivo increase in number relative to the number originally present in a culture. In another embodiment, monocytes, macrophages, or dendritic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector: As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Immune cell: As used herein, the term “immune cell,” refers to a cell that is involved in an immune response, e.g., promotion of an immune response. Examples of immune cells include, but are not limited to, macrophages, monocytes, dendritic cells, neutrophils, eosinophils, mast cells, platelets, large granular lymphocytes, Langerhans' cells, natural killer (NK) cells, T-lymphocytes, or B-lymphocytes. A source of immune cells (e.g., macrophages, monocytes, or dendritic cells) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Modified: As used herein, the term “modified” refers to a changed state or structure of a molecule or cell of the invention. Molecules may be modified in many ways, including chemically, structurally, and functionally. Cells may be modified through the introduction of nucleic acids.

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof).

In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Overexpressed tumor antigen: As used herein, the term “overexpressed” tumor antigen or “overexpression” of a tumor antigen refers to an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Signal transduction pathway: As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

Specifically binds: As used herein, the term “specifically binds,” with respect to an antigen binding domain, such as an antibody agent, refers to an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

Stimulation: As used herein, the term “stimulation,” refers to a primary response induced by binding of a stimulatory molecule (e.g., an FcR complex, a TLR complex, or a TCR/CD3 complex), for example, with its cognate ligand thereby mediating a signal transduction event, such as, but not limited to, signal transduction via Fc receptor machinery or via a synthetic CAR. Stimulation can mediate altered expression of certain molecules, such as downregulation of TGF-beta, and/or reorganization of cytoskeletal structures, and the like. As used herein, the term “stimulatory molecule,” refers to a molecule of a monocyte, macrophage, or dendritic cell that specifically binds with a cognate stimulatory ligand present on an antigen presenting cell. In some embodiments, a stimulatory molecule comprises an FcR extracellular domain comprising a CD64 (FcγRI), CD32a (FcγRIIa), CD32b (FcγRIIb), CD32c, CD16a (FcγRIIIa), CD16b (FcγRIIIb), FcERI, FcERII, FcaRI (CD89) or CD40 domain. In some embodiments, a stimulatory molecule comprises a TLR extracellular domain comprising a TLR1, TLR2, TLR3, TLR4, TLR5, TLR6, TLR7, TLR8, or TLR9 domain. As used herein, the term “stimulatory ligand,” refers to a ligand that when present on an antigen presenting cell (e.g., an aAPC, a macrophage, a dendritic cell, a B-cell, and the like) or tumor cell can specifically bind with a cognate binding partner (referred to herein as a “stimulatory molecule”) on a monocyte, macrophage, or dendritic cell thereby mediating a response by the immune cell, including, but not limited to, activation, initiation of an immune response, proliferation, and the like. Stimulatory ligands are well-known in the art and encompass, inter alia, Toll-like receptor (TLR) ligand, an anti-toll-like receptor antibody, an agonist, and an antibody for a monocyte/macrophage receptor. In addition, cytokines, such as interferon-gamma, are potent stimulants of macrophages.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially purified: As used herein, the term “substantially purified”, for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, an antibody (or fragment thereof) or a CAR.

Target site: As used herein, the term “target site” or “target sequence” refers to a genomic nucleic acid sequence that defines a portion of a nucleic acid to which a binding molecule may specifically bind under conditions sufficient for binding to occur.

T cell receptor: As used herein, the term “T cell receptor” or “TCR” refers to a complex of membrane proteins that participate in the activation of T cells in response to the presentation of antigen. A TCR is responsible for recognizing antigens bound to major histocompatibility complex molecules. A TCR comprises a heterodimer of an alpha (a) and beta (β) chain, although in some cells the TCR comprises gamma and delta (γ/δ) chains. TCRs may exist in alpha/beta and gamma/delta forms, which are structurally similar but have distinct anatomical locations and functions. Each chain comprises two extracellular domains, a variable and constant domain. In some embodiments, a TCR may be modified on any cell comprising a TCR, including, for example, a helper T cell, a cytotoxic T cell, a memory T cell, regulatory T cell, natural killer T cell, and gamma delta T cell.

Therapeutic: As used herein, the term “therapeutic” refers to a treatment and/or prophylaxis. A therapeutic effect is obtained by suppression, remission, or eradication of one or more symptoms or features of a disease state.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering to an immune cell (e.g., a monocyte, macrophage, or dendritic cell) or contacting an immune cell with a modulator of a pathway activated by in vitro transcribed mRNA.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

JAK/STAT proteins are an integral part of cytokine signaling. The recruitment of intracellular Janus kinases (JAKs) and signal transducer and activator of transcription proteins (STATs) to activated cytokine receptors can have a range of effects, such as induction of cytokine secretion, differentiation, and shift of cell polarization/phenotype. The present disclosure provides for the incorporation of JAK/STAT signaling domains into chimeric antigen receptor (CAR) architecture. These CARs comprising JAK and STAT binding domains allow for recruitment of JAK, followed by phosphorylation of STAT molecules and the activation of their downstream transcriptional outputs. This, in turn, can allow modified cells comprising one or more such CARs to leverage, for example, native JAK-STAT signaling machinery, in order to modulate the response of modified cells to particular stimuli (e.g., allowing a cell to react to a normally inflammatory stimuli as if it were a non-inflammatory stimuli, or vice versa).

Macrophages and monocytes are powerful modulators of the immune response. Macrophages can generally adopt either a pro-inflammatory (M1) or an anti-inflammatory (M2) phenotype. A precise balance of M1/M2 macrophages is important in the body's response to disease and injury, and various diseases include dysregulated M1/M2 phenotypes. For example, macrophages in the tumor microenvironment (TME) are often biased toward an M2 phenotype that safeguards the tumor, while M1 macrophages in atherosclerotic tissue typically promote plaque progression. Methods that allow external control over macrophage phenotype are thus promising therapeutic strategies, whether by repolarizing existing macrophages or by delivering macrophages of a desired phenotype (e.g. the delivery of M1 macrophages to the TME or M2 macrophages to atherosclerotic tissue). Natural systems use cytokines as potent regulators of M1/M2 phenotype, and thus manipulated cytokine signaling networks represent an appealing system for engineering macrophages and monocytes.

### Immune Cells

The present disclosure provides, among other things, modified immune cells (e.g., stem cells, macrophages, monocytes, or dendritic cells) comprising a chimeric antigen receptor (CAR) as described herein. In some embodiments, a modified immune cell of the present disclosure further comprises a membrane-tethered cytokine as described herein.

In some embodiments, a population of immune cells as described herein comprises stem cells, monocytes, macrophages, dendritic cells, and/or precursors thereof. In some embodiments, a population of immune cells comprises a substantially purified population of stem cells, monocytes, macrophages, or dendritic cells, or a cell line.

In some embodiments, an immune cell is activated, e.g., an immune cell exhibits increased cytokine production, chemokine production, phagocytosis, cell signaling, target cell killing, and/or antigen presentation in response to a particular stimuli, e.g., relative to an inactive cell. In some embodiments, an activated immune cell exhibits changes in gene expression, e.g., an induction of pro-inflammatory gene expression, e.g., relative to an inactive cell. In some embodiments, an activated immune cell exhibits changes in gene expression, e.g., an induction of anti-inflammatory gene expression, e.g., relative to an inactive cell. In certain embodiments, activated immune cells are undergoing cell division. In some embodiments, targeted effector activity of an immune cell is enhanced by inhibition of CD47 and/or SIRPa activity. CD47 and/or SIRPa activity may be inhibited by treating an immune cell with an anti-CD47 or anti-SIRPa antibody or by any method known to those skilled in the art.

In some embodiments, immune cells (e.g., stem cells, macrophages, monocytes, or dendritic cells) are obtained (e.g., isolated) from a subject. Immune cells may be autologous or sourced from allogeneic or universal donors. Cells can be obtained from a number of sources including peripheral blood mononuclear cells, bone marrow, lymph node tissue, spleen tissue, umbilical cord, tumors, and/or induced pluripotent stem cells, such as embryonic stem cells (ESCs). In certain embodiments, cells can be obtained from a unit of blood collected from a subject using any number of separation techniques known to a skilled artisan, such as Ficoll separation. In some embodiments, cells from circulating blood of a subject are obtained by apheresis or leukapheresis. Cells collected by apheresis may be washed to remove a plasma fraction and resuspended in a variety of buffers (e.g., phosphate buffered saline (PBS)) or culture media). In some embodiments, enrichment of immune cells (e.g. monocytes) comprises plastic adherence. In some embodiments, following enrichment, differentiation of immune cells (e.g. monocytes) comprises stimulation with GM-CSF. In some embodiments, a composition comprising blood cells (e.g., monocytes, lymphocytes, platelets, plasma, and/or red blood cells), such as a leukapheresis composition (e.g., a leukopak) is used for enrichment. In some embodiments, a leukapheresis composition (e.g., a leukopak) comprises a sample from a healthy human donor. In certain embodiments, apheresis of immune cells (e.g. monocytes) is followed by mobilization with GM-CSF. In certain embodiments, selection of immune cells (e.g., monocytes) comprises CD14 positive selection using microbeads (e.g., MACS® MicroBeads on a CliniMACS Prodigy device). In some embodiments, an immune cell precursor (e.g., precursors to macrophages, monocytes, or dendritic cells including, but not limited to induced pluripotent stem cells, or iPSCs) is used in compositions and methods described herein. Immune cell precursors may be differentiated in vivo or ex vivo into immune cells. Non-limiting examples of precursor immune cells include hematopoietic stem cells, common myeloid progenitors, myeloblasts, monoblasts, promonocytes, or intermediates thereof. For example, induced pluripotent stem cells may be used to generate monocytes, macrophages, and/or dendritic cells. Induced pluripotent stem cells (iPSCs) may be derived from normal human tissue, such as peripheral blood, fibroblasts, skin, keratinocytes, or renal epithelial cells. Autologous, allogeneic, or universal donor iPSCs could be differentiated toward a myeloid lineage (e.g., a monocyte, macrophage, dendritic cell, or precursor thereof).

Immune cells (e.g., stem cells, macrophages, monocytes, or dendritic cells) as described herein can be isolated from peripheral blood, for example, by lysing red blood cells and depleting lymphocytes and red blood cells, such as by centrifugation through a PERCOLL™ gradient. Alternatively, immune cells can be isolated from umbilical cord tissue. A specific subpopulation of immune cells can be further isolated by positive or negative selection techniques. In some embodiments, immune cells can be depleted of cells expressing certain antigens, including, but not limited to, CD34, CD3, CD4, CD8a, CD56, CD66b, CD19, or CD20. In some embodiments, enrichment of an immune cell population, for example, by negative selection can be accomplished using a combination of antibodies directed to surface markers unique to the negatively selected cells. By way of non-limiting example, cell selection can also comprise negative magnetic immunoadherence or flow cytometry that uses a cocktail of monoclonal antibodies directed to cell surface markers present on negatively selected cells.

During isolation of a desired population of immune cells (e.g., stem cells, macrophages, monocytes, or dendritic cells) as described herein by positive or negative selection, immune cell concentration and surface (e.g., particles, such as beads) can be varied. It may be desirable to significantly decrease volume in which beads and cells are mixed together to ensure maximum contact area of cells and beads.

In some embodiments, prior to administration, immune cells (e.g., stem cells, macrophages, monocytes, or dendritic cells) as described herein (e.g., comprising a CAR as described herein) are treated with a pro-inflammatory agent. In some embodiments, treatment with a pro-inflammatory agent promotes an anti-inflammatory (i.e., M2) phenotype in immune cells as described herein. In some embodiments, a pro-inflammatory agent comprises or is a pro-inflammatory cytokine. In some embodiments, a pro-inflammatory agent comprises or is IFN-α, IFN-β, IFN-λ, IFN-γ, TNF-α, IL-6, IL-1b, IL-2, IL-8, IL-12, IL-15, IL-18, IL-17, IL-1a, IL-3, IL-21, IL-33, IL-23, IL-37, or IL-36. In some embodiments, a pro-inflammatory agent comprises or is an IFN-γ agonist (e.g., IFN-γ).

In some embodiments, prior to administration, immune cells (e.g., stem cells, macrophages, monocytes, or dendritic cells) as described herein (e.g., comprising a CAR as described herein) are treated with an anti-inflammatory agent. In some embodiments, treatment with an anti-inflammatory agent promotes a pro-inflammatory (i.e., M1) phenotype in immune cells as described herein. In some embodiments, an anti-inflammatory agent comprises or is an anti-inflammatory cytokine. In some embodiments, an anti-inflammatory agent comprises or is IL-10, TGF-β, IL-4, IL-13, IL-11, or IL-35. In some embodiments, an anti-inflammatory agent comprises or is an IL-10 agonist (e.g., IL-10).

In some embodiments, immune cells (e.g., stem cells, macrophages, monocytes, or dendritic cells) as described herein (e.g., comprising a CAR as described herein) are administered to a subject in combination with a pro-inflammatory agent. In some embodiments, immune cells (e.g., stem cells, macrophages, monocytes, or dendritic cells) as described herein (e.g., comprising a CAR as described herein) are administered to a subject substantially simultaneously, before, or after a pro-inflammatory agent. In some embodiments, administration with a pro-inflammatory agent promotes an anti-inflammatory (i.e., M2) phenotype in immune cells as described herein. In some embodiments, a pro-inflammatory agent comprises or is an IFN-γ agonist (e.g., IFN-γ).

In some embodiments, immune cells (e.g., stem cells, macrophages, monocytes, or dendritic cells) as described herein (e.g., comprising a CAR as described herein) are administered to a subject in combination with an anti-inflammatory agent. In some embodiments, immune cells (e.g., stem cells, macrophages, monocytes, or dendritic cells) as described herein (e.g., comprising a CAR as described herein) are administered to a subject substantially simultaneously, before, or after an anti-inflammatory agent. In some embodiments, administration with an anti-inflammatory agent increases anti-tumor activity of immune cells as described herein. In some embodiments, administration with an anti-inflammatory agent promotes a pro-inflammatory (i.e., M1) phenotype in immune cells as described herein. In some embodiments, an anti-inflammatory agent comprises or is an IL-10 agonist (e.g., IL-10).

### Macrophages

Macrophages are immune cells specialized for detection, phagocytosis, and destruction of target cells, such as pathogens or tumor cells. Macrophages are potent effectors of the innate immune system and are capable of at least three distinct anti-tumor functions: 1) phagocytosis of dead and dying cells, microorganisms, cancer cells, cellular debris, or other foreign substances; 2) cytotoxicity against tumor cells; and 3) presentation of tumor antigens to orchestrate an adaptive anti-tumor immune response.

Accumulating evidence suggests that macrophages are abundant in the tumor microenvironment of numerous cancers and can adopt a number of phenotypes that are collectively referred to as tumor-associated macrophages (TAMs). The immunosuppressive nature of the tumor microenvironment typically results in more M2-like TAMs, which further contribute to the general suppression of anti-tumor immune responses. Recent studies, however, have identified that TAMs are able to be “reprogrammed” via pro-inflammatory signals, and that the switch from a M2 phenotype to a more M1 phenotype is associated with productive anti-tumor immune responses. Inducing endogenous TAMs to switch to M1-type cells and engineering macrophages that cannot be subverted into M2 would greatly enhance anti-tumor immunotherapy and represent a significant advance in the field.

In some embodiments, a macrophage comprises or is an undifferentiated or M0 macrophage. In certain embodiments, a macrophage comprises or expresses one, two, three, four, five, or six of CD14, CD16, CD64, CD68, CD71, or CCR5. Exposure to various stimuli can induce M0 macrophages to polarize into several distinct populations, which may be identified by macrophage phenotype markers, cytokine production, and/or chemokine secretion.

In some embodiments, a macrophage comprises or is a polarized macrophage. Under classical conditions of activation, M0 macrophages can be exposed to pro-inflammatory signals, such as LPS, IFNγ, and GM-CSF, and polarize into pro-inflammatory (i.e., M1) macrophages. Generally, pro-inflammatory (M1) macrophages are associated with pro-inflammatory immune responses, such as Th1 and Th17 T cell responses. Exposure to other stimuli can polarize macrophages into a diverse group of “alternatively activated” or anti-inflammatory (i.e., M2) macrophages.

In some embodiments, a macrophage comprises or is a pro-inflammatory (M1) macrophage. In some embodiments, a macrophage expresses one or more markers of pro-inflammatory (M1) macrophages (e.g., 1, 2, 3, 4, 5, 6, 7, 8, 9, 10, 11, 12, 13, 14, 15, 16, 17, or 18 of CD86, CD80, MHC II, IL-1R, TLR2, TLR4, iNOS, SOCS3, CD83, PD-L1, CD69, MHC I, CD64, CD32, CD16, ILIR, a IFIT family member, or an ISG family member).

In some embodiments, a macrophage comprising or expressing a CAR as described herein secretes relatively high levels of one or more inflammatory cytokines (e.g., 1, 2, 3, 4, 5, 6, 7, 8, 9, 10, 11, or 12 of IL-1, TNF, IL-12, IL-18, IL-23, IFNα, IFNβ, IFNγ, IL-2, IL-6, IL-8, or IL33) or chemokines (e.g., one or both of CC or CXC chemokines) (e.g., 1, 2, 3, 4, 5, 6, 7, 8, 9, 10, 11, 12, 13, 14, 15, or 16 of the CXC chemokines; e.g., 1, 2, 3, 4, 5, 6, 7, 8, 9, 10, 11, 12, 13, 14, 15, 16, 17, 18, 19, 20, 21, 22, 23, 24, 25, 26, 27, or 28 of the CC chemokines; eg., one of the CX3C chemokines, e.g., one or both of the C chemokines), e.g., relative to a macrophage without a CAR as described herein. In some embodiments, a macrophage comprising or expressing a CAR as described herein stimulates an immune response and/or inflammation, e.g., relative to a macrophage without a CAR as described herein.

In some embodiments, a macrophage comprises or is an anti-inflammatory (M2) macrophage (e.g., an M2a, M2b, M2c, and M2d macrophage). An M2a macrophage can be induced by IL-4, IL-13, and/or fungal infection. An M2b macrophage can be induced by IL-1R ligands, an immune complex, and/or LPS. An M2c macrophage can be induced by IL-10 and/or TGFβ. An M2d macrophage can be induced by IL-6 and/or adenosine. In some embodiments, a macrophage comprising or expressing a CAR as described herein decreases an immune response in a subject, e.g., relative to a macrophage without a CAR as described herein. In some embodiments, a macrophage expresses one or more markers of anti-inflammatory (M2) macrophages (e.g., one, two, or three of CD206, CD163, or CD209). In some embodiments, a macrophage comprising or expressing a CAR as described herein exhibits increased secretion of one or more anti-inflammatory cytokines (e.g., one or both of IL-10 or TGFβ), e.g., relative to a macrophage without a CAR as described herein.

In some embodiments, a macrophage comprises at least one upregulated pro-inflammatory (M1) marker and/or at least one downregulated anti-inflammatory (M2) marker as compared to a control macrophage that does not comprise a CAR as described herein and/or the same macrophage before delivery of a CAR as described herein. In some embodiments, at least one pro-inflammatory (M1) marker (e.g., HLA DR, CD86, CD80, PD-L1, CD83, CD69, MHC I, CD64, CD32, CD16, IL1R, an IFIT family member, and/or an ISG family member) is upregulated in a macrophage. In some embodiments, at least one anti-inflammatory (M2) marker (e.g., CD206, CD163, and/or CD209) is downregulated in a macrophage.

In some embodiments, a macrophage comprising or expressing a CAR as described herein exhibits increased phagocytosis, e.g., relative to a macrophage without a CAR as described herein. In some embodiments, a macrophage comprising or expressing a CAR as described herein exhibits increased cytotoxicity against a tumor cell, e.g., relative to a macrophage without a CAR as described herein. In some embodiments, a macrophage comprising or expressing a CAR as described herein exhibits increased tumor antigen presentation (e.g., post-phagocytosis presentation) and/or increased antigen processing, e.g., relative to a macrophage without a CAR as described herein. In some embodiments, a macrophage comprising or expressing at a CAR as described herein exhibits increased tumor killing (e.g., by phagocytosis, lysis, apoptosis, or production of tumor killing cytokines (e.g., TNFα)), e.g., relative to a macrophage without a CAR as described herein.

In some embodiments, a macrophage comprising or expressing a CAR as described herein exhibits one or both of increased expression of one or more genes typically associated with increased effector function (e.g., phagocytosis, targeted cellular cytotoxicity, antigen presentation, or cytokine secretion) (e.g., CD80, CD86, MHC-I, MHC-II, CD40, 41BBL, TNF, IFN-α, IFN-β, IFN-γ, IL2, IL12, IL6, IL8, IL1b, and/or CXCL12) or decreased expression of one or more genes typically associated with decreased effector function (e.g., phagocytosis, targeted cellular cytotoxicity, antigen presentation, or cytokine secretion) (e.g., CD163, CD206, TGFβ, IL-10, and/or IL4), e.g., relative to a macrophage without a CAR as described herein. In some embodiments, a macrophage comprising or expressing a CAR as described herein exhibits increased production of ROS, e.g., relative to a macrophage without a CAR as described herein. In some embodiments, a macrophage comprising or expressing a CAR as described herein exhibits metabolic reprogramming (e.g., of an interferon signaling pathway, TH1 pathway, PTEN signaling, PI3K signaling, MTOR signaling, TLR signaling, CD40 signaling, 41BB signaling, 41BBL signaling, macrophage maturation signaling, dendritic cell maturation signaling, CD3-zeta signaling, FcR γ signaling, CD64 signaling, CD32a signaling, CD32c signaling, CD16a signaling, TLR1 signaling, TLR2 signaling, TLR3 signaling, TLR4 signaling, TLR5 signaling, TLR6 signaling, TLR7 signaling, TLR8 signaling, TLR9 signaling, ALK signaling, AXL signaling, DDR2 signaling, EGFR signaling, EphA1 signaling, INSR signaling, cMET signaling, MUSK signaling, PDGFR signaling, PTK7 signaling, RET signaling, ROR1 signaling, ROS1 signaling, RYK signaling, TIE2 signaling, TRK signaling, VEGFR signaling, CD40 signaling, CD19 signaling, CD20 signaling, 41BB signaling, CD28 signaling, OX40 signaling, GITR signaling, TREM-1 signaling, TREM-2 signaling, DAP12 signaling, MR signaling, ICOS signaling, MyD88 signaling, V/I/LxYxxL/V signaling, SIRPa signaling, CD45 signaling, Siglec-10 signaling, PD1 signaling, SHP-1 signaling, SHP-2 signaling, KIR-2DL signaling, KIR-3DL signaling, NKG2A signaling, CD170 signaling, CD33 signaling, BTLA signaling, CD32b signaling, SIRPβ signaling, CD22 signaling, PIR-B signaling, and/or LILRB1 signaling), e.g., relative to a macrophage without a CAR as described herein. In some embodiments, a macrophage comprising or expressing a CAR as described herein exhibits induction of cell survival mechanisms, e.g., relative to a macrophage without a CAR as described herein. In some embodiments, a macrophage comprising or expressing a CAR as described herein exhibits induction of cell death mechanisms, e.g., relative to a macrophage without a CAR as described herein. In some embodiments, a macrophage comprising or expressing a CAR as described herein exhibits one, two, three, four, or five of increased resistance to phagocytic checkpoints, increased expression of chemokine receptors to aid in trafficking, increased expression of chemokines to recruit other immune cells, increased expression of ECM degrading enzymes (e.g., MMPs to degrade tumor ECM and/or exhibit anti fibrotic activity), and/or increased proliferation, e.g., relative to a macrophage without a CAR as described herein. In some embodiments, a macrophage comprising or expressing a CAR as described herein exhibits one, two, three, or four of improved duration of CAR expression, improved stability of the CAR on the cell surface, increased level of CAR expression, and/or decreased background activity of the CAR, e.g., relative to a macrophage without a CAR as described herein.

In some embodiments, a macrophage comprising or expressing a CAR as described herein decreases one or more signs and/or symptoms of an infection (e.g., of an infectious agent) in a subject, e.g., relative to a macrophage without a CAR as described herein. In some embodiments, an infectious agent comprises or is a virus, a protozoa (e.g., trypanosome, malaria, or toxoplasma), a bacteria (e.g., mycobacterium, salmonella, or listeria), a fungi (e.g., Candida), or a combination thereof. In some embodiments, a virus comprises hepatitis virus (e.g., hepatitis A, hepatitis B, hepatitis C, or hepatitis E), retrovirus, human immunodeficiency virus (e.g., HIV1 or HIV2), T cell leukemia virus, a Lymphotropic virus (e.g., HTLV1 or HTLV2), herpes simplex virus (e.g., herpes simplex virus type 1 or type 2), Epstein-Barr virus, cytomegalovirus, varicella-zoster virus, poliovirus, measles virus, Rubella virus, Japanese encephalitis virus, mumps virus, influenza virus, adenovirus, enterovirus, rhinovirus, coronavirus (e.g., severe acute respiratory syndrome (SARS) virus, Middle East respiratory syndrome (MERS) virus, or severe acute respiratory syndrome coronavirus 2 (SARS-CoV2)), Ebola virus, West Nile virus, or a variant or combination thereof.

In some embodiments, a macrophage comprising or expressing a CAR as described herein decreases formation and/or degrades existing aggregates via phagocytosis of at least one protein aggregate in a subject (e.g., a subject having a neurodegenerative disease, an inflammatory disease, a cardiovascular disease, a fibrotic disease, amyloidosis, or a combination thereof), e.g., relative to a macrophage without a CAR as described herein. In some embodiments, a neurodegenerative disease is selected from the group consisting of tauopathy, a-synucleopathy, presenile dementia, senile dementia, Alzheimer's disease, progressive supranuclear palsy (PSP), Pick's disease, primary progressive aphasia, frontotemporal dementia, corticobasal dementia, Parkinson's disease, dementia with Lewy bodies, Down's syndrome, multiple system atrophy, amyotrophic lateral sclerosis (ALS), Hallervorden-Spatz syndrome, polyglutamine disease, trinucleotide repeat disease, and prion disease. In some embodiments, an inflammatory disease is selected from the group consisting of systemic lupus erythematosus, vasculitis, rheumatoid arthritis, periodontitis, ulcerative colitis, sinusitis, asthma, tuberculosis, Crohn's disease, chronic infection, hereditary periodic fever, a malignancy, systemic vasculitides, cystic fibrosis, bronchiectasis, epidermolysis bullosa, cyclic neutropenia, an immunodeficiency, Muckle-Wells (MWS) disease, and Familiar Mediterranean Fever (FMF). In some embodiments, amyloidosis is selected from the group consisting of Primary Amyloidosis (AL), Secondary Amyloidosis (AA), Familial Amyloidosis (ATTR), Beta-2 Microglobulin Amyloidosis, Localized Amyloidosis, Heavy Chain Amyloidosis (AH), Light Chain Amyloidosis (AL), Primary Systemic Amyloidosis, ApoAI Amyloidosis, ApoAII Amyloidosis, ApoAIV Amyloidosis, Apolipoprotein C2 Amyloidosis, Apolipoprotein C3 Amyloidosis, Corneal lactoferrin amyloidosis, Transthyretin-Related Amyloidosis, Dialysis amyloidosis, Fibrinogen amyloidosis, Lect2 amyloidosis (ALECT2), and Lysozyme amyloidosis. In some embodiments, a cardiovascular disease is selected from the group consisting of atherosclerosis, coronary artery disease, peripheral artery disease, hypertensive heart disease, metabolic syndrome, hypertension, cerebrovascular disease, and heart failure. In some embodiments, a fibrotic disease is selected from the group consisting of pulmonary fibrosis, idiopathic pulmonary fibrosis, cirrhosis, cystic fibrosis, scleroderma, cardiac fibrosis, radiation-induced lung injury, steatohepatitis, glomerulosclerosis, interstitial lung disease, liver fibrosis, mediastinal fibrosis, retroperitoneal cavity fibrosis, bone marrow fibrosis, and skin fibrosis.

### Monocytes

Monocytes are multipotent cells that circulate in the blood, bone marrow, and spleen, and generally do not proliferate when in a steady state. Monocytes can vary in size significantly in the range of about 10-30 μm in diameter. A ratio of nucleus to cytoplasm for a monocyte can range from about 2:1 to about 1:1. Typically, monocytes comprise chemokine receptors and pathogen recognition receptors that mediate migration from blood to tissues, such as during an infection. Monocytes can produce inflammatory cytokines, take up cells and/or toxic molecules, and differentiate into dendritic cells or macrophages.

In some embodiments, a monocyte comprises or expresses one or more phenotypic markers. Exemplarily phenotypic markers for human monocyte cells include, but are not limited to, CD9, CD11b, CD11c, CDw12, CD13, CD15, CDw17, CD31, CD32, CD33, CD35, CD36, CD38, CD43, CD49b, CD49e, CD49f, CD63, CD64, CD65s, CD68, CD84, CD85, CD86, CD87, CD89, CD91, CDw92, CD93, CD98, CD101, CD102, CD111, CD112, CD115, CD116, CD119, CDwl2lb, CDw123, CD127, CDw128, CDw131, CD147, CD155, CD156a, CD157, CD162 CD163, CD164, CD168, CD171, CD172a, CD180, CD206, CD131a1, CD213 2, CDw210, CD226, CD281, CD282, CD284, and CD286. Exemplarily phenotypic markers for mouse monocyte cells include, but are not limited to, CD11a, CD11b, CD16, CD18, CD29, CD31, CD32, CD44, CD45, CD49d, CD115, CD116, Cdw131, CD281, CD282, CD284, CD286, F4/80, and CD49b. In certain embodiments, monocytes comprise one, two, or three of CD11b, CD14, or CD16. In certain embodiments, monocytes comprise CD14+CD16-monocytes, CD14+CD16+ monocytes, or CD14-CD16+ monocytes.

In some embodiments, a monocyte differentiates into a macrophage. In some embodiments, a monocyte differentiates into a dendritic cell (DC). Monocytes can be differentiated into macrophages or DCs by any technique known in the art. For example, differentiation of monocytes into macrophages can be induced by macrophage colony stimulating factor (M-CSF). Differentiation of monocytes into DCs can be induced by granulocyte-macrophage colony stimulating factor (GM-CSF) in combination with IL-4.

In some embodiments, a monocyte comprising or expressing a CAR as described herein exhibits increased secretion of one or more cytokines (e.g., one, two, three, four, five, six, or seven of TNF, IL-12, IFN, GM-CSF, G-CSF, M-CSF, or IL-1), e.g., relative to a monocyte without a CAR as described herein. In some embodiments, a monocyte comprising or expressing a CAR as described herein exhibits increased phagocytosis, e.g., relative to a monocyte without a CAR as described herein. In some embodiments, a monocyte comprising or expressing a CAR as described herein exhibits enhanced survival, e.g., relative to a monocyte without a CAR as described herein. In some embodiments, a monocyte comprising or a CAR as described herein exhibits enhanced differentiation into macrophages (e.g., M1 or M2 macrophages), e.g., relative to a monocyte without a CAR as described herein. In some embodiments, a monocyte comprising or expressing a CAR as described herein as described herein exhibits enhanced differentiation into DCs (e.g., resident or migrating DCs and/or in lymphoid and non-lymphoid tissue), e.g., relative to a monocyte without a CAR as described herein. In some embodiments, a monocyte comprising or expressing a CAR as described herein exhibits increased cytotoxicity against a tumor cell, e.g., relative to a monocyte without a CAR as described herein. In some embodiments, a monocyte comprising or expressing a CAR as described herein exhibits increased tumor antigen presentation (e.g., post-phagocytosis presentation) and/or increased antigen processing, e.g., relative to a monocyte without a CAR as described herein. In some embodiments, a monocyte comprising or expressing a CAR as described herein exhibits increased tumor killing (e.g., by phagocytosis, lysis, apoptosis, or production of tumor killing cytokines (e.g., TNFα), e.g., relative to a monocyte without a CAR as described herein.

In some embodiments, a monocyte comprising or expressing a CAR as described herein exhibits one or both of increased expression of one or more genes typically associated with increased effector function (e.g., phagocytosis, targeted cellular cytotoxicity, antigen presentation, or cytokine secretion) or decreased expression of one or more genes typically associated with decreased effector function (e.g., phagocytosis, targeted cellular cytotoxicity, antigen presentation, or cytokine secretion), e.g., relative to a monocyte without a CAR as described herein. In some embodiments, a monocyte comprising or expressing a CAR as described herein exhibits increased production of ROS, e.g., relative to a monocyte without a CAR as described herein. In some embodiments, a monocyte comprising or expressing a CAR as described herein exhibits metabolic reprogramming, e.g., relative to a monocyte without a CAR as described herein. In some embodiments, a monocyte comprising or expressing a CAR as described herein exhibits induction of cell survival mechanisms, e.g., relative to a monocyte without a CAR as described herein. In some embodiments, a monocyte comprising or expressing a CAR as described herein exhibits induction of cell death mechanisms, e.g., relative to a monocyte without a CAR as described herein. In some embodiments, a monocyte comprising or expressing a CAR as described herein exhibits one, two, three, four, or five of increased resistance to phagocytic checkpoints, increased expression of chemokine receptors to aid in trafficking, increased expression of chemokines to recruit other immune cells, increased expression of ECM degrading enzymes (e.g., MMPs to degrade tumor ECM and/or exhibit anti fibrotic activity), or increased proliferation, e.g., relative to a monocyte without a CAR as described herein. In some embodiments, a monocyte comprising or expressing a CAR as described herein exhibits one, two, three, or four of improved duration of CAR expression, improved stability of the CAR on the cell surface, increased level of CAR expression, and/or decreased background activity of the CAR, e.g., relative to a monocyte without a CAR as described herein.

### Dendritic Cells

Dendritic cells (DCs) are bone marrow-derived, specialized antigen presenting cells that are involved in initiating immune responses and maintaining tolerance of the immune system to self-antigens. Dendritic cells may be found in both lymphoid and non-lymphoid organs and are generally thought to arise from lymphoid or myeloid lineages.

In some embodiments, a DC comprises or expresses one or more phenotypic markers. Exemplarily phenotypic markers for DCs include, but are not limited to, CD11c, CD83, CD1a, CD1c, CD141, CD207, CLEC9a, CD123, CD85, CD180, CD187, CD205, CD281, CD282, CD284, CD286 and partially CD206, CD207, CD208 and CD209.

Immature DCs can be characterized by a high capacity for antigen capture, but relatively low T cell stimulatory capability. Inflammatory mediators promote DC maturation. Once DCs reach the mature stage, there is a dramatic change in properties relative to immature DCs, such as a decrease in antigen capture ability and/or an increased ability to stimulate T cells. In some embodiments, a DC comprises or is an immature DC. In other embodiments, a DC comprises or is a mature DC.

Without wishing to be bound by theory, it is believed that modification of a DC cell to comprise or express a CAR as described herein as described herein can allow mature DCs to simultaneously exhibit increased antigen capture ability and T cell stimulation, e.g., relative to a DC without a CAR as described herein. In some embodiments, a DC comprising or expressing a CAR as described herein mediates tumor antigen presentation, e.g., increased tumor antigen presentation relative to a DC without a CAR as described herein. In some embodiments, a DC comprising or expressing a CAR as described herein mediates tumor T cell stimulation, e.g., increased T cell stimulation relative to a DC without a CAR as described herein.

In some embodiments, a DC comprising or expressing a CAR as described herein exhibits increased secretion of one or more cytokines (e.g., one, two, three, four, five, six, or seven of TNF, IL-12, IFN, GM-CSF, G-CSF, M-CSF, or IL-1), e.g., relative to a DC without a CAR as described herein. In some embodiments, a DC comprising or expressing a CAR as described herein exhibits increased phagocytosis, e.g., relative to a DC without a CAR as described herein. In some embodiments, a DC comprising or expressing a CAR as described herein exhibits increased tumor antigen presentation (e.g., post-phagocytosis presentation), increased antigen processing, increased antigen cross presentation, increased T cell priming, and/or stimulation of T cells, e.g., relative to a DC without a CAR as described herein.

In some embodiments, a DC comprising or expressing a CAR as described herein exhibits one or both of increased expression of favorable genes or decreased expression of unfavorable genes, e.g., relative to a DC without a CAR as described herein. In some embodiments, a DC comprising or expressing a CAR as described herein exhibits increased production of ROS, e.g., relative to a DC without a CAR as described herein. In some embodiments, a DC comprising or expressing a CAR as described herein exhibits metabolic reprogramming, e.g., relative to a DC without a CAR as described herein. In some embodiments, a DC comprising or expressing a CAR as described herein exhibits induction of cell survival mechanisms, e.g., relative to a DC without a CAR as described herein.

In some embodiments, a DC comprising or expressing a CAR as described herein exhibits induction of cell death mechanisms, e.g., relative to a DC without a CAR as described herein. In some embodiments, a DC comprising or expressing a CAR as described herein exhibits one, two, three, four, or five of increased resistance to phagocytic checkpoints, increased expression of chemokine receptors to aid in trafficking, increased expression of chemokines to recruit other immune cells, increased expression of ECM degrading enzymes (e.g., MMPs to degrade tumor ECM and/or exhibit anti fibrotic activity), or increased proliferation, e.g., relative to a DC without a CAR as described herein. In some embodiments, a DC comprising or expressing a CAR as described herein exhibits one, two, three, or four of improved duration of CAR expression, improved stability of the CAR on the cell surface, increased level of CAR expression, and/or decreased background activity of the CAR, e.g., relative to a DC without a CAR as described herein.

### Methods of Immune Cell Modification

The present disclosure provides, among other things, methods for modifying an immune cell (e.g., a stem cell, monocyte, macrophage, or dendritic cell) comprising delivering to the immune cell a nucleic acid construct comprising one or more nucleic acids encoding a CAR as described herein or a fragment thereof into an immune cell. Methods can comprise delivering to an immune cell (e.g., a stem cell, monocyte, macrophage, or dendritic cell), a nucleic acid construct comprising one or more nucleic acids encoding: a CAR comprising an extracellular domain, a transmembrane domain and one or more intracellular moieties, wherein the one or more intracellular moieties comprise a first JAK binding domain (or a portion thereof) and a first STAT binding domain (or a portion thereof). In some embodiments, a nucleic acid construct of the present disclosure further comprises one or more nucleic acids encoding a CAR and/or a membrane-tethered cytokine.

In some embodiments, the present disclosure provides methods of producing a modified immune cell (e.g., a stem cell, monocyte, macrophage, or dendritic cell) in a subject comprising administering to a subject a composition as described herein comprising: (a) one or more nucleic acid molecules, wherein the one or more intracellular moieties comprise a first JAK binding domain (or a portion thereof) and a first STAT binding domain (or a portion thereof), at least one membrane-tethered cytokine, or combinations thereof as described herein, and (b) a delivery vehicle. Accordingly, in some embodiments, following administration of the composition, one or more nucleic acid molecules are translated in an immune cell (e.g., stem cell, macrophage, monocyte, or dendritic cell) to produce a modified immune cell comprising the CAR and/or membrane-tethered cytokine. In some embodiments, the modified immune cell comprising the CAR and/or membrane-tethered cytokine possesses targeted effector activity.

### Delivery Methods

A nucleic acid construct comprising one or more nucleic acid sequences encoding at least one CAR comprising one or more intracellular moieties, wherein the one or more intracellular moieties comprise a first JAK binding domain (or a portion thereof) and a first STAT binding domain (or a portion thereof), at least one membrane-tethered cytokine, or combinations thereof as described herein, can be introduced into an immune cell (e.g., a stem cell, macrophage, monocyte, or dendritic cell) by physical, chemical, or biological methods. In some embodiments, the present disclosure provides methods for modifying an immune cell comprising producing a modified immune cell (e.g., a stem cell, monocyte, macrophage, or dendritic cell) ex vivo. In some embodiments, the present disclosure provides methods for modifying an immune cell comprising producing a modified immune cell (e.g., a stem cell, monocyte, macrophage, or dendritic cell) in a subject (i.e., in vivo).

Physical methods for introducing a nucleic acid construct as described herein into an immune cell (e.g., a stem cell, macrophage, monocyte, or dendritic cell) can comprise electroporation, calcium phosphate precipitation, lipofection, particle bombardment, microinjection, or a combination thereof. A nucleic acid construct can be introduced into immune cells using commercially available methods, including electroporation (Amaxa Nucleofector-II® (Amaxa Biosystems, Cologne, Germany), ECM 830 BTX (Harvard Instruments, Boston, Mass.) Gene Pulser II® (BioRad, Denver, Colo.), or Multiporator® (Eppendort, Hamburg Germany)). A nucleic acid construct can also be introduced into immune cells using mRNA transfection, e.g., cationic liposome-mediated transfection, lipofection, polymer encapsulation, peptide-mediated transfection, or biolistic particle delivery systems, such as “gene guns” (See, e.g., Nishikawa, et al. Hum Gene Ther., 12 (8): 861-70 (2001), which is hereby incorporated by reference in its entirety).

Biological methods for introducing a nucleic acid construct as described herein into an immune cell (e.g., a stem cell, macrophage, monocyte, or dendritic cell) include use of DNA and RNA vectors. In one embodiment, a vector comprises a plasmid vector, a viral vector, a transposon, a retrotransposon (e.g., piggyback, sleeping beauty), a site directed insertion vector (e.g., CRISPR, Zn finger nucleases, TALEN), suicide expression vector, or another vector known in the art. Viral vectors, and especially retroviral vectors, have become widely used for inserting genes into mammalian cells (e.g., human cells). Viral vectors can also be derived from lentivirus, poxviruses, herpes simplex virus I, adenoviruses (e.g. Ad5f35), or adeno-associated viruses (See, e.g., U.S. Pat. Nos. 5,350,674 and 5,585,362, which are hereby incorporated by reference in their entirety). Retroviral vectors, such as lentivirus, are suitable tools to achieve long-term gene transfer that allow for long-term, stable integration of a transgene and its propagation in daughter cells. In some embodiments, a lentiviral vector is packaged with a Vpx protein (e.g., as described in International Publication No. WO 2017/044487, which is hereby incorporated by reference in its entirety). In some embodiments, Vpx comprises a virion-associated protein (e.g., an accessory protein for viral replication). In some embodiments, a Vpx protein is encoded by human immunodeficiency virus type 2 (HIV-2). In some embodiments, a Vpx protein is encoded by simian immunodeficiency virus (SIV). In some embodiments, an immune cell as described herein (e.g., a stem cell, macrophage, monocyte, or dendritic cell) is transfected with a lentiviral vector packaged with a Vpx protein. In some embodiments, Vpx inhibits at least one antiviral factor of an immune cell as described herein (e.g., a stem cell, macrophage, monocyte, or dendritic cell). In some embodiments, a lentiviral vector packaged with a Vpx protein exhibits increased transfection efficiency of an immune cell as described herein (e.g., a stem cell, macrophage, monocyte, or dendritic cell), e.g., relative to a lentiviral vector not packaged with a Vpx protein. In some embodiments, an immune cell as described herein (e.g., a stem cell, macrophage, monocyte, or dendritic cell) is one or both of electroporated or transfected with at least one VPX mRNA prior to transfection with a viral vector (e.g., an adenoviral vector, e.g., an Ad2 vector or an Ad5 vector (e.g., Ad5f35 adenoviral vector, e.g., a helper-dependent Ad5F35 adenoviral vector)).

Chemical means for introducing a nucleic acid construct as described herein into an immune cell (e.g., a stem cell, macrophage, monocyte, or dendritic cell) include colloidal dispersion systems, macromolecule complexes, nanocapsules, microspheres, beads, and lipid-based systems (e.g., oil-in-water emulsions, micelles, mixed micelles, nanoparticles, liposomes, and lipofectamine-nucleic acid complexes).

An exemplary system for delivery of a nucleic acid construct as described herein is a lipid-based system. A nucleic acid construct as described herein may be encapsulated in an aqueous interior of a liposome, interspersed within a lipid bilayer, attached to a liposome via a linking molecule, attached to a lipid nanoparticle (LNP) via a linking molecule, entrapped in a liposome, entrapped in an LNP, complexed with a liposome, complexed with an LNP, dispersed in a solution or suspension comprising a lipid, mixed with a lipid, complexed with a micelle, or otherwise associated with a lipid. Lipids for use in methods described herein may be naturally occurring or synthetic lipids. Lipids can also be obtained from commercial sources. For example, dimyristyl phosphatidylcholine can be obtained from Sigma (St. Louis, MO); dicetyl phosphate can be obtained from K & K Laboratories (Plainview, NY); cholesterol can be obtained from Calbiochem-Behring; and dimyristyl phosphatidylglycerol can be obtained from Avanti Polar Lipids, Inc. (Birmingham, AL.). Stock solutions of lipids in chloroform or chloroform/methanol can be stored at about-20° C. In some embodiments, a lipid-based system may comprise one or more lipids that facilitate targeting of the composition to a desired cell type or cell types (e.g., stem cells, monocytes, macrophages, or dendritic cells). In some embodiments, a delivery vehicle allows a composition to be preferentially taken up (e.g. endocytosed, phagocytosed) by an immune cell (e.g., stem cell, monocyte, macrophage, or dendritic cell) relative to a composition that does not comprise the delivery vehicle.

### Targeting Moieties

In some embodiments, a delivery vehicle may comprise one or more targeting moieties. In some embodiments, a targeting moiety may facilitate passive targeting of a composition to a desired target. In some embodiments, a targeting moiety may facilitate active targeting of a composition to a desired target.

In some embodiments, a targeting moiety may be or comprise one of more of an antibody (e.g., a monoclonal antibody, a polyclonal antibody, a synthetic antibody, a human antibody, a humanized antibody, a non-human antibody) or any fragment thereof, for example an scFv, an aptamer, a darpin, a centyrin, a naturally occurring or synthetic receptor, an affibody, or other engineered protein recognition molecule, for example, to bind to one or more of CD14, CD11b, CD163, CD206, CD33, CD209. In some embodiments, a targeting moiety may be or comprise a small molecule.

In some embodiments, a targeting moiety may be or comprise a particular lipid or combination of hydrophobic entities, for example, present in or forming an exterior surface of a liposome or lipid nanoparticle (e.g., for targeting to a particular cell type or cell types).

### Nucleic Acid Molecules

In some embodiments of the present disclosure, one or more nucleic acid molecules are or comprise DNA. In some embodiments of the present disclosure, one or more nucleic acid molecules are or comprise messenger RNA (mRNA). In some embodiments, mRNA according to the present disclosure may be synthesized as unmodified or modified mRNA. Typically, mRNAs are modified to enhance stability. Modifications of mRNA can include, for example, modifications of the nucleotides of the RNA. A modified mRNA according to the present disclosure can thus include, for example, backbone modifications, sugar modifications or base modifications. In some embodiments, a step of modifying an mRNA comprises causing the mRNA to include a modified nucleotide, an alteration to the 5′ or 3′ untranslated region (UTR), a cap structure, and/or a poly(A) tail.

In some embodiments, mRNAs of the present disclosure (e.g., mRNAs encoding CARs) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which comprises replacing the phosphodiester linkage by other anionic, cationic or neutral groups.

In some embodiments, mRNAs of the present disclosure (e.g., mRNAs encoding CARs) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, mRNAs of the present disclosure (e.g., mRNAs encoding CARs) comprise modified nucleotide comprising pseudouridine (PsU), 5-methoxyuridine (5moU), 5-methylcytidine/pseudouridine (5meC PsU), N1-methyl-pseudouridine (NlmPsU), or combinations thereof.

In some embodiments, mRNAs of the present disclosure (e.g., mRNAs encoding CARs) may contain modifications of the bases of the nucleotides (base modifications). A modified nucleotide which contains a base modification is also called a base-modified nucleotide.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs of the present disclosure (e.g., mRNAs encoding CARs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′ triphosphate linkage; and the 7-nitrogen of guanine is then methylated by a methyltransferase. Examples of cap structures include, but are not limited to, m7G (5′)ppp(5′ (A,G (5′)ppp(5′) A and G (5′)ppp(5′) G. In some embodiments, a cap comprises a Cap0 structure. A cap0 structures lack a 2′-O-methyl residue of the ribose attached to bases 1 and 2. In some embodiments, a cap comprises an AGCap1 structure. An AGCap1 structures has a 2′-O-methyl residue at base 2. In some embodiments, a cap comprises a Cap2 structure. Cap2 structures have a 2′-O-methyl residue attached to both bases 2 and 3. In some embodiments, a cap structure comprises AGCap1, m6AGCap1, or Anti-Reverse Cap Analog (ARCA). In some embodiments, a modified mRNA of the present disclosure comprises an m6AGCap1 and modified nucleotides comprising pseudouridine (PsU).

In some embodiments, mRNAs of the present disclosure (e.g., mRNAs encoding CARs) include a 3′ poly(A) tail structure. A poly(A) tail on the 3′ terminus of mRNA typically includes about 10 to 400 adenosine nucleotides (SEQ ID NO: 148) (e.g., about 100 to 400 adenosine nucleotides, about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly(C) tail on the 3′ terminus of mRNA typically include about 10 to 200 cytosine nucleotides (SEQ ID NO: 149) (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). A poly(C) tail may be added to a poly(A) tail or may be a substitute for the poly(A) tail.

In some embodiments, mRNAs of the present disclosure (e.g., mRNAs encoding CARs) include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA's stability of location in a cell, or one or more binding sites for miRNAs. In some embodiments, a 3′ untranslated region may be between 50 and 500 nucleotides in length or longer.

### Administration of Additional Payloads

In some embodiments, methods of the present disclosure comprise one or more steps of treating an immune cell (e.g., a stem cell, macrophage, monocyte, or dendritic cell) during the process of modifying the immune cell. In some embodiments, methods of the present disclosure comprise one or more steps of administering to a subject an additional payload for modulating an immune cell (e.g., a stem cell, macrophage, monocyte, or dendritic cell) during the process of modifying the immune cell. In some embodiments, a composition may comprise one or more additional payloads. In some embodiments, a composition may comprise one or more additional payloads in the same delivery vehicle as one or more nucleic acid molecules. In some embodiments, a composition may comprise one or more additional payloads in a different delivery vehicle than the one used with one or more nucleic acid molecules.

In some embodiments, methods of the present disclosure comprise a step of treating an immune cell (e.g., a stem cell, macrophage, monocyte, or dendritic cell) with a modulator of a pathway activated by in vitro transcribed mRNA. In some embodiments, an additional payload may be or comprise a modulator of a pathway activated by in vitro transcribed mRNA. In vitro transcribed (IVT) mRNA is recognized by various endosomal innate immune receptors (Toll-like receptor 3 (TLR3), TLR7 and TLR8) and cytoplasmic innate immune receptors (protein kinase RNA-activated (PKR), retinoic acid-inducible gene I protein (RIG-I), melanoma differentiation-associated protein 5 (MDA5) and 2′-5′-oligoadenylate synthase (OAS)). Signaling through these different pathways results in inflammation associated with type 1 interferon (IFN), tumor necrosis factor (TNF), interleukin-6 (IL-6), IL-12 and the activation of cascades of transcriptional programs. Overall, these create a pro-inflammatory microenvironment poised for inducing specific immune responses. Moreover, downstream effects such as slow-down of translation by eukaryotic translation initiation factor 2a (eIF2a) phosphorylation, enhanced RNA degradation by ribonuclease L (RNaseL), and overexpression and inhibition of replication of self-amplifying mRNA are of relevance for the pharmacokinetics and pharmacodynamics of IVT mRNA.

In some embodiments, a modulator of a pathway activated by in vitro transcribed mRNA comprises an RNase inhibitor. In some embodiments, a modulator of a pathway activated by in vitro transcribed mRNA comprises an RNaseL, RNase T2 or RNasel inhibitor. In some embodiments, a modulator of a pathway activated by in vitro transcribed mRNA comprises an RNaseL inhibitor. In some embodiments, an RNaseL inhibitor comprises sunitinib. In some embodiments, an RNaseL inhibitor comprises ABCE1.

In some embodiments, treating an immune cell (e.g., a stem cell, macrophage, monocyte, or dendritic cell) with an RNaseL inhibitor increases mRNA stability in a modified immune cell relative to mRNA stability in a modified immune cell of the same type that was not treated with an RNaseL inhibitor. In some embodiments, treating an immune cell (e.g., a stem cell, macrophage, monocyte, or dendritic cell) with an RNaseL inhibitor increases CAR expression in a modified immune cell relative to CAR expression in a modified immune cell of the same type that was not treated with an RNaseL inhibitor. In some embodiments, treating an immune cell (e.g., a stem cell, macrophage, monocyte, or dendritic cell) with an RNaseL inhibitor increases membrane-tethered cytokine expression in a modified immune cell relative to membrane-tethered cytokine expression in a modified immune cell of the same type that was not treated with an RNaseL inhibitor. In some embodiments, treating an immune cell (e.g., a stem cell, macrophage, monocyte, or dendritic cell) with an RNaseL inhibitor increases effector activity in a modified immune cell relative to effector activity in a modified immune cell of the same type that was not treated with an RNaseL inhibitor.

In some embodiments, administering to a subject an RNaseL inhibitor increases mRNA stability in a modified immune cell (e.g., a stem cell, macrophage, monocyte, or dendritic cell) relative to mRNA stability in a modified immune cell of the same type in a subject that that was not administered an RNaseL inhibitor. In some embodiments, administering to a subject an RNaseL inhibitor increases CAR expression in a modified immune cell (e.g., a stem cell, macrophage, monocyte, or dendritic cell) relative to CAR expression in a modified immune cell of the same type in a subject that was not administered an RNaseL inhibitor. In some embodiments, administering to a subject an RNaseL inhibitor increases membrane-tethered cytokine expression in a modified immune cell (e.g., a stem cell, macrophage, monocyte, or dendritic cell) relative to membrane-tethered cytokine expression in a modified immune cell of the same type in a subject that was not administered an RNaseL inhibitor. In some embodiments, administering to a subject an RNaseL inhibitor increases effector activity in a modified immune cell (e.g., a stem cell, macrophage, monocyte, or dendritic cell) relative to effector activity in a modified immune cell of the same type in a subject that was not administered an RNaseL inhibitor.

In some embodiments of the present disclosure, a step of treating an immune cell (e.g., a stem cell, macrophage, monocyte, or dendritic cell) occurs before a step of delivering an mRNA to the immune cell. In some embodiments of the present disclosure, a step of administering an additional payload to a subject occurs before a step of administering a composition comprising an mRNA to the subject.

In some embodiments, methods of the present disclosure comprise a step of culturing an immune cell (e.g., a stem cell, macrophage, monocyte, or dendritic cell) with a cytokine or immune stimulating recombinant protein. In some embodiments, methods of the present disclosure comprise a step of administering to a subject a cytokine or immune stimulating recombinant protein. In some embodiments, a cytokine comprises IFN-α, IFN-β, IFN-γ, TNFα, IL-6, STNGL, LPS, a CD40 agonist, a 4-1BB ligand, recombinant 4-1BB, a CD19 agonist, a TLR agonist (e.g., TLR-1, TLR-2, TLR-3, TLR-4, TLR-5, TLR-6, TLR-7, TLR-8 or TLR-9), TGF-β (e.g., TGF-1, TGF-β2, or TGF-β3), a glucocorticoid, an immune complex, interleukin-1 alpha (IL-1a), IL-1B, IL-2, IL-3, IL-4, IL-5, IL-6, IL-7, IL-9, IL-10, IL-12, IL-13, IL-14, IL-15, IL-16, IL-17, IL-18, IL-20, granulocyte-macrophage colony-stimulating factor (GM-CSF), granulocyte colony-stimulating factor (G-CSF), Leukemia inhibitory factor (LIF), oncostatin M (OSM), TNF-β, CD154, lymphotoxin beta (LT-B), an A proliferation-inducing ligand (APRIL), CD70, CD153, glucocorticoid-induced TNF receptor ligand (GITRL), tumor necrosis factor superfamily member 14 (TNFSF14), OX40L (CD252), TALL-1 (Tumor necrosis factor ligand superfamily member 13B-TNFSF13B), TNF-related apoptosis-inducing ligand (TRAIL), TNF-related weak inducer of apoptosis (TWEAK), TNF-related activation-induced cytokine (TRANCE), erythropoietin (Epo), thyroid peroxidase precursor (Tpo), FMS-related tyrosine kinase 3 ligand (FLT-3L), stem cell factor (SCF), macrophage colony-stimulating factor (M-CSF), merozoite surface protein (MSP), a Nucleotide-binding oligomerization domain-containing protein (NOD) ligand (e.g., NOD1, NOD2, or NOD1/2 agonists), a RIG-I-like receptor (RLR) ligand (e.g., 5′ppp-dsRNA, 3p-hpRNA, Poly(I:C), or Poly(dA:dT)), a C-type lectin receptor (CLR) ligand (e.g., curdlan, β-glucan, HKCA, laminarin, pustulan, scleroglucan, WGP dispersible, WGP soluble, zymosan, zymosan depleted, furfurman, b-GlcCer, GlcC14C18, HKMT, TDB, TDB-HS15, or TDM), a cyclic dinucleotide sensor ligand (e.g., C-Gas agonist or stimulator of interferon gene (STING) ligand), an inflammasome inducer (e.g., alum, ATP, CPPD crystals, hemozoin, MSU crystals, Nano-SiO2, Nigericin, or TDB), an aryl hydrocarbon (AhR) ligand (e.g., FICZ, indirubin, ITE, or L-kynurenine), an alpha-protein kinase 1 (ALPK1) ligand, a multi-PRR ligand, an NFKB/NFAT activator (e.g., concavalin A, ionomycin, PHA-P, or PMA) or combinations thereof. In some embodiments, a cytokine comprises IFN-β.

In some embodiments of the present disclosure, a step of culturing an immune cell (e.g., a stem cell, macrophage, monocyte, or dendritic cell) occurs after a step of delivering an mRNA to the immune cell. In some embodiments of the present disclosure, a step of administering to a subject a cytokine or immune stimulating recombinant protein occurs after a step of administering a composition comprising an mRNA to the subject.

In some embodiments, culturing a modified immune cell (e.g., a stem cell, macrophage, monocyte, or dendritic cell) with a cytokine or immune stimulating recombinant protein increases the viability of the modified immune cell relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases protein (e.g., CAR and/or membrane-tethered cytokine) expression in the modified immune cell relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases longevity of protein (e.g., CAR and/or membrane-tethered cytokine) expression relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases effector activity of the modified immune cell relative to a modified immune cell of the same type that was not cultured with the cytokine or immune stimulating recombinant protein. In some embodiments, culturing a modified immune cell (e.g., a stem cell, macrophage, monocyte, or dendritic cell) with a cytokine or immune stimulating recombinant protein increases pro-inflammatory (M1) polarization of the modified immune cell relative to a modified immune cell of the same type that was not cultured with the cytokine or immune stimulating recombinant protein.

In some embodiments, administering to a subject a cytokine or immune stimulating recombinant protein increases the viability of a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases protein (e.g., CAR and/or membrane-tethered cytokine) expression of a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases longevity of protein (e.g., CAR and/or membrane-tethered cytokine) expression in a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases effector activity of a modified immune cell (e.g., a stem cell, macrophage, monocyte, or dendritic cell) in the subject relative to a modified immune cell of the same type in a subject that was not administered the cytokine or immune stimulating recombinant protein. In some embodiments, administering to a subject a cytokine or immune stimulating recombinant protein increases pro-inflammatory (M1) polarization of a modified immune cell (e.g., a stem cell, macrophage, monocyte, or dendritic cell) in the subject relative to a modified immune cell of the same type in a subject that was not administered the cytokine or immune stimulating recombinant protein.

### Methods of Altering the Inflammatory Phenotype of a Population of Cells

In some embodiments, methods of the present disclosure comprise altering the inflammatory phenotype of a population of cells. In some embodiments, methods of altering the inflammatory phenotype of a population of cells comprises contacting the population of cells with a modified immune cell (e.g., a stem cell, macrophage, monocyte, or dendritic cell) as described herein. In some embodiments, a population of cells comprises macrophages, monocytes, dendritic cells, T cells, NK cells, or combinations thereof.

In some embodiments, the inflammatory phenotype of the population of cells is altered from anti-inflammatory to non-activated. In some embodiments, the inflammatory phenotype of the population of cells is altered from pro-inflammatory to non-activated. In some embodiments, the inflammatory phenotype of the population of cells is altered from anti-inflammatory to pro-inflammatory. In some embodiments, the inflammatory phenotype of the population of cells is altered from pro-inflammatory to anti-inflammatory.

### Modified Immune Cells

In some embodiments, a modified immune cell (e.g., a stem cell, macrophage, monocyte, or dendritic cell) is made by methods of the present disclosure. In some embodiments, a modified immune cell comprises a CAR. In some embodiments a modified immune cell comprises one or more nucleic acids encoding a CAR. In some embodiments, a CAR comprises an extracellular domain, a transmembrane domain and one or more intracellular moieties. In some embodiments, one or more intracellular moieties comprise a first JAK binding domain or a portion thereof and a first STAT binding domain or a portion thereof. In some embodiments, one or more intracellular moieties further comprise a first intracellular domain or a portion thereof.

In some embodiments, one or more intracellular moieties are, in order (from cellular membrane proximal to distal): (i) a first JAK binding domain or portion thereof, a first STAT binding domain or portion thereof, and a first intracellular domain or portion thereof; (ii) a first STAT binding domain or portion thereof, a first JAK binding domain or portion thereof, and a first intracellular domain or portion thereof; (iii) a first JAK binding domain or portion thereof, a first intracellular domain or portion thereof, and a first STAT binding domain or portion thereof; (iv) a first STAT binding domain or portion thereof, a first intracellular domain or portion thereof, and a first JAK binding domain or portion thereof; (v) a first intracellular domain or portion thereof, a first JAK binding domain or portion thereof, and a first STAT binding domain or portion thereof; (vi) a first intracellular domain or portion thereof, a first STAT binding domain or portion thereof, and a first JAK binding domain or portion thereof; (vii) a first JAK binding domain or portion thereof and a first STAT binding domain or portion thereof; or (viii) a first STAT binding domain or portion thereof and a first JAK binding domain or portion thereof.

In some embodiments, one or more intracellular moieties further comprise: a second JAK binding domain or a portion thereof, a second STAT binding domain or a portion thereof, a second intracellular domain or a portion thereof, or combinations thereof. In some embodiments, a modified immune cell (e.g., a stem cell, macrophage, monocyte, or dendritic cell) of the present disclosure comprises a CAR comprising one or more intracellular moieties comprising one, two, three, four, five or six of: a first JAK binding domain or portion thereof, a second JAK binding domain or portion thereof, a first STAT binding domain or portion thereof, a second STAT binding domain or portion thereof, a first intracellular domain or portion thereof and/or a second intracellular domain or portion thereof in any order (from cellular membrane proximal to distal).

In some embodiments, a first JAK binding domain or portion thereof is derived from a first cytokine receptor. In some embodiments, a first STAT binding domain or portion thereof is derived from a second cytokine receptor. In some embodiments, a second JAK binding domain or portion thereof is derived from a third cytokine receptor. In some embodiments, a second STAT binding domain or portion thereof is derived from a fourth cytokine receptor. In some embodiments, a first cytokine receptor and a second cytokine receptor are different cytokine receptors (e.g., different types of cytokine receptors, e.g., a first cytokine receptor comprises a G-CSF receptor or portion thereof and a second cytokine receptor comprises an IFNγ receptor or portion thereof). In some embodiments, a first cytokine receptor and a second cytokine receptor are the same cytokine receptor (e.g., the same type of cytokine receptor, e.g., an IL-10 receptor or portion thereof). In some embodiments, a third cytokine receptor and a fourth cytokine receptor are different cytokine receptors. In some embodiments, a third cytokine receptor and a fourth cytokine receptor are the same cytokine receptor. In some embodiments, a first cytokine receptor and a third cytokine receptor are different cytokine receptors. In some embodiments, a first cytokine receptor and a third cytokine receptor are the same cytokine receptor. In some embodiments, a second cytokine receptor and a fourth cytokine receptor are different cytokine receptors. In some embodiments, a second cytokine receptor and a fourth cytokine receptor are the same cytokine receptor.

In some embodiments, a first cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a second cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a third cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a fourth cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a first JAK binding domain or portion thereof binds JAK1, JAK2, JAK3, TYK2, or combinations thereof. In some embodiments, a second JAK binding domain or portion thereof binds JAK1, JAK2, JAK3, TYK2, or combinations thereof. In some embodiments, a first STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof. In some embodiments, a second STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof.

In some embodiments, a first intracellular domain or portion thereof is or comprises a CD34, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL 7R, IL8R, IL9R, IL10R, IL11R, IL12R, IL13R, IL14R, IL15R, IL17R, IFNaR, IFNgR, TNFR, CSFIR, CSF2R, Dap10, CD36, Dectin-1, ICOSL, or Syk intracellular domain, a portion of any of the foregoing, or combinations thereof. In some embodiments, a second intracellular domain or portion thereof is or comprises a CD34, FcRY, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL 12R, IL13R, IL 14R, IL15R, IL17R, IFNaR, IFNgR, TNFR, CSFIR, CSF2R, Dap10, CD36, Dectin-1, ICOSL, or Syk intracellular domain, a portion of any of the foregoing, or combinations thereof. In some embodiments, a first intracellular domain comprise a CD3 intracellular domain or an FcRγ intracellular domain.

In some embodiments, an extracellular domain is or comprises an scFv, VHH antibody, centyrin, darpin, or nanobody.

In some embodiments, a transmembrane domain is or comprises a CD8a, CD28, CD40, MyD88 CD64, CD32a, CD32c, CD16a, CD3zeta, ICOS, Dectin-1, DNGR1, SLAMF7, TRL1, TLR2, TLR3, TRL4, TLR5, TLR6, TLR7, TLR8, or TLR9 transmembrane domain.

In some embodiments, a CAR further comprises an extracellular leader domain. In some embodiments, an extracellular leader domain comprises a CD8a extracellular leader domain. In some embodiments, a CAR further comprises an extracellular hinge domain. In some embodiments, an extracellular hinge domain comprises: a CD8a extracellular hinge domain, a CD28 extracellular hinge domain, a DNGR-1 extracellular hinge domain, a Dectin-1 extracellular hinge domain, or an IgG4 extracellular hinge domain.

In some embodiments, a CAR has or comprises: an amino acid sequence selected from Table 2; an amino acid sequence that differs from a sequence selected from Table 2 by no more than five substitutions, additions, or deletions; or an amino acid sequence that is at least 80% identical to a sequence selected from Table 2.

In some embodiments, a first cytokine receptor is selected from Table 1. In some embodiments, a second cytokine receptor is selected from Table 1. In some embodiments, a third cytokine receptor is selected from Table 1. In some embodiments, a fourth cytokine receptor is selected from Table 1.

TABLE 1
Cytokine Receptors and Intracellular Output Signals

| | | | | | | GeneID for |
|---|---|---|---|---|---|---|
| Output | Intracellular | | Primary | Additional | General | Ligand |
| Signaling | Signaling | Intracellular | Signal | Signal | Macrophage | Binding |
| Family | Output | Domain From: | Transducer | Transducer | Polarization | Receptor |
| JAK/STAT | IL-10 | IL-10Ra | STAT3 | — | M2 | 3587 |
| JAK/STAT | IL-4/IL-13 | IL4Ra | STAT6 | — | M2 | 3566 |
| JAK/STAT | IL-7/TSLP | IL7Ra | STAT5 | STAT1, | M2 | 3575 |
| | | | | STAT3 | | |
| JAK/STAT | IL-9 | IL9Ra | STAT5 | STAT1, | M2 | 3581 |
| | | | | STAT3 | | |
| JAK/STAT | IL-21 | IL21Ra | STAT5 | STAT1, | M2 | 50615 |
| | | | | STAT3 | | |
| JAK/STAT | IL-2/IL-15 | IL2Rb | STAT5 | — | M1 | 3560 |
| JAK/STAT | IL-6/IL-11 | gp130 | STAT3 | STAT1 | M2 | 3572 |
| JAK/STAT | IL-6 | IL6Ra | STAT3 | STAT1 | M2 | 3570 |
| JAK/STAT | IL-11 | IL11Ra | STAT3 | STAT1 | M2 | 3590 |
| JAK/STAT | GCSF | GCSFR | STAT3 | — | M2 | 1441 |
| JAK/STAT | IL-3 | IL3Ra | STAT5 | — | | 3563 |
| JAK/STAT | IL-5 | IL5Ra | STAT5 | — | | 3568 |
| JAK/STAT | GM-CSF | GMCSFRa | STAT5 | — | M1 | 1438 |
| JAK/STAT | IL-3/5/GM-CSF | CSF2Rb | STAT5 | — | | 1439 |
| JAK/STAT | LIF | LIFRb | STAT3 | — | | 3977 |
| JAK/STAT | IL-31 | IL31Ra | STAT3 | — | M1 | 133396 |
| JAK/STAT | CNTF/CT-1 | CNTFR | STAT3 | — | | 1271 |
| JAK/STAT | IL-27 | IL27Ra | STAT3 | — | | 9466 |
| JAK/STAT | EPO | EPOR | STAT5 | STAT1 | M1 | 2057 |
| JAK/STAT | GH | GHR | STAT5 | STAT1 | M1 | 2690 |
| JAK/STAT | PRL | PRLR | STAT5 | — | | 5618 |
| JAK/STAT | IFN-α/β/ω/ε/κ | IFNAR2 | STAT1/2 | — | M1 | 3455 |
| JAK/STAT | IFN-γ | IFNGR1 | STAT1 | — | M1 | 3459 |
| JAK/STAT | IFN-λ1/λ2/λ3 | IL28R | STAT1/2 | — | M1 | 163702 |
| JAK/STAT | IL-26/19/20/24 | IL20Ra | STAT3 | — | M2 | 53832 |
| JAK/STAT | IL-22/20/24 | IL22R | STAT3 | — | M2 | 58985 |
| JAK/STAT | STAT1 | (minimal | STAT1 | — | M1 | NA |
| | | STAT1 binding | | | | |
| | | motif pYxxP) | | | | |
| JAK/STAT | STAT3 | (minimal | STAT3 | — | M2 | NA |
| | | STAT3 binding | | | | |
| | | motif pYxxQ) | | | | |
| JAK/STAT | STAT5 | (minimal | STAT5 | — | M1 | NA |
| | | STAT5a binding | | | | |
| | | motif pYxxL) | | | | |
| JAK/STAT | STAT6 | (minimal | STAT6 | — | M2 | NA |
| | | STAT6 binding | | | | |
| | | motif pYxxF) | | | | |
| TNFRSF/TRAF | CD40 | CD40 | TRAF2/3/6 | — | M1 | 958 |
| TNFRSF/TRAF | 4-1BB | 4-1BB | TRAF1/2 | — | M1 | 3604 |
| TLR/TRAF | TLR4 | MyD88 | IRAK1/4 | TRAF6 | M1 | 4615 |
| TNFRSF/TRAF | OX40 | OX40 | TRAF2 | — | | 7293 |
| TNFRSF/TRAF | CD30 | CD30 | TRAF2/3 | — | | 943 |
| TNFRSF/TRAF | TNFα | TNFR1 | TRAF2/5 | — | M1 | 7132 |
| TNFRSF/TRAF | TNFα | TNFR2 | TRAF1/2 | 14-3-3e | M1 | 7133 |
| STAT | FLT3L | FLT3 | STAT5 | — | | 2322 |
| DAP12 | TREM1 | TREM1 | DAP12 | — | M1 | 54210 |
| DAP12 | TREM2 | TREM2 | DAP10/12 | — | M2 | 54209 |
| Receptor | Tyro3 | Tyro3 | PI3K | SOCS1/3 | M2 | 7301 |
| tyrosine kinase | | | | | | |
| Receptor | Axl | Axl | PI3K | SOCS1/3 | M2 | 558 |
| tyrosine kinase | | | | | | |
| Receptor | MerTK | MerTK | PI3K | SOCS1/3 | M2 | 10461 |
| tyrosine kinase | | | | | | |

In some embodiments, a first cytokine receptor comprises an amino acid sequence at least 80% identical to a sequence selected from Table 4. In some embodiments, a second cytokine receptor comprises an amino acid sequence at least 80% identical to a sequence selected from Table 4. In some embodiments, a third cytokine receptor comprises an amino acid sequence at least 80% identical to a sequence selected from Table 4. In some embodiments, a fourth cytokine receptor comprises an amino acid sequence at least 80% identical to a sequence selected from Table 4.

In some embodiments, a first cytokine receptor comprises a nucleic acid sequence at least 80% identical to a sequence selected from Table 5. In some embodiments, a second cytokine receptor comprises a nucleic acid sequence at least 80% identical to a sequence selected from Table 5. In some embodiments, a third cytokine receptor comprises a nucleic acid sequence at least 80% identical to a sequence selected from Table 5. In some embodiments, a fourth cytokine receptor comprises a nucleic acid sequence at least 80% identical to a sequence selected from Table 5.

In some embodiments, a modified immune cell (e.g., a stem cell, macrophage, monocyte, or dendritic cell) of the present disclosure comprises a CAR comprising an extracellular domain that binds a tumor antigen, such as an antigen that is specific for a tumor or cancer of interest. In some embodiments a tumor antigen comprises one or more antigenic cancer epitopes. In some embodiments, a tumor antigen comprises CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a, anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1). In certain embodiments, a tumor antigen comprises ERBB2 (Her2/neu). In certain embodiments, a tumor antigen comprises PSMA. In certain embodiments, a tumor antigen comprises Mesothelin.

In some embodiments, a modified immune cell (e.g., a stem cell, macrophage, monocyte, or dendritic cell) of the present disclosure comprises a membrane-tethered cytokine. In some embodiments, modified immune cells comprising membrane-tethered cytokines can stimulate receptors on neighboring cells. In some embodiments, a membrane-tethered cytokine comprises an extracellular domain and a membrane tether. In some embodiments, an extracellular domain is or comprises a pro-inflammatory cytokine. In some embodiments, an extracellular domain is or comprises an anti-inflammatory cytokine. In some embodiments, an extracellular domain is or comprises IFN-β. In some embodiments, a membrane tether is or comprises: a B7 transmembrane domain (TMD); a B7 TMD with a matrix metalloproteinase (MMP) linker; a glycosylphosphatidylinositol (GPI) anchor; or a GPI anchor with a CD28 spacer. In some embodiments, a membrane tether can release from the modified immune cell upon binding of the extracellular domain with a receptor expressed by another cell.

In some embodiments, a modified immune cell (e.g., a stem cell, macrophage, monocyte, or dendritic cell) comprising a modified mRNA encoding a CAR as described herein exhibits increased viability relative to a modified immune cell of the same type comprising unmodified mRNA encoding a CAR as described herein. In some embodiments, a modified immune cell (e.g., a stem cell, macrophage, monocyte, or dendritic cell) comprising a modified mRNA encoding a CAR as described herein exhibits increased expression of an mRNA encoding a CAR as described herein relative to a modified immune cell of the same type comprising unmodified mRNA encoding the CAR. In some embodiments, a modified immune cell (e.g., a stem cell, macrophage, monocyte, or dendritic cell) comprising a modified mRNA encoding a CAR as described herein exhibits increased CAR expression relative to a modified immune cell of the same type comprising unmodified mRNA encoding the CAR. In some embodiments, a modified immune cell (e.g., a stem cell, macrophage, monocyte, or dendritic cell) comprising a modified mRNA encoding a CAR as described herein exhibits increased longevity of a mRNA encoding a CAR as described herein relative to a modified immune cell of the same type comprising unmodified mRNA encoding the CAR. In some embodiments, a modified immune cell (e.g., a stem cell, macrophage, monocyte, or dendritic cell) comprising a modified mRNA encoding a CAR as described herein exhibits increased longevity of the CAR relative to a modified immune cell of the same type comprising unmodified mRNA encoding the CAR. In some embodiments, a modified immune cell (e.g., a stem cell, macrophage, monocyte, or dendritic cell) comprising a modified mRNA encoding a CAR as described herein exhibits increased effector activity relative to a modified immune cell of the same type comprising unmodified mRNA encoding the CAR. In some embodiments, a modified immune cell (e.g., a stem cell, macrophage, monocyte, or dendritic cell) comprising a modified mRNA encoding a CAR as described herein exhibits increased pro-inflammatory (M1) polarization relative to a modified immune cell of the same type comprising unmodified mRNA encoding the CAR.

In some embodiments, a modified immune cell (e.g., a stem cell, macrophage, monocyte, or dendritic cell) comprising a CAR as described herein maintains a pro-inflammatory phenotype over time. In some embodiments, a modified immune cell (e.g., a stem cell, macrophage, monocyte, or dendritic cell) comprising a CAR as described herein maintains a pro-inflammatory phenotype at least 4 hours, 2 days, 4 days, 7 days, 14 days, and/or 28 days after an immune cell is modified with a nucleic acid encoding the CAR.

In some embodiments, a modified immune cell (e.g., a stem cell, macrophage, monocyte, or dendritic cell) comprising a CAR as described herein maintains an anti-inflammatory phenotype over time. In some embodiments, a modified immune cell (e.g., a stem cell, macrophage, monocyte, or dendritic cell) comprising a CAR as described herein maintains an anti-inflammatory phenotype at least 4 hours, 2 days, 4 days, 7 days, 14 days, and/or 28 days after an immune cell is modified with a nucleic acid encoding the CAR.

In some embodiments, a modified immune cell (e.g., a stem cell, macrophage, monocyte, or dendritic cell) comprising a CAR as described herein maintains a pro-inflammatory phenotype and/or otherwise resists subversion when challenged by anti-inflammatory cytokines. In some embodiments, the sensitivity of a modified immune cell to environmental cytokines is measured by generating a dose-response curve of pro-inflammatory markers by treating modified immune cells comprising a CAR as described herein with increasing concentrations of anti-inflammatory cytokines. In some embodiments, the sensitivity of a modified immune cell to environmental cytokines is measured by generating a dose-response curve of pro-inflammatory markers by treating modified immune cells comprising a CAR as described herein with increasing concentrations of pro-inflammatory cytokines.

In some embodiments, a modified immune cell (e.g., a stem cell, macrophage, monocyte, or dendritic cell) comprising a CAR as described herein maintains an anti-inflammatory phenotype and/or otherwise resists subversion when challenged by pro-inflammatory cytokines. In some embodiments, the sensitivity of a modified immune cell to environmental cytokines is measured by generating a dose-response curve of anti-inflammatory markers by treating modified immune cells comprising a CAR as described herein with increasing concentrations of pro-inflammatory cytokines. In some embodiments, the sensitivity of a modified immune cell to environmental cytokines is measured by generating a dose-response curve of anti-inflammatory markers by treating modified immune cells comprising a CAR as described herein with increasing concentrations of anti-inflammatory cytokines.

In some embodiments, a modified immune cell (e.g., a stem cell, macrophage, monocyte, or dendritic cell) comprising a CAR as described herein has minimal effects on neighboring cells. In some embodiments, a modified immune cell (e.g., a stem cell, macrophage, monocyte, or dendritic cell) comprising a CAR as described herein has significant effects on neighboring cells. In some embodiments, the effect of a modified immune cell (e.g., a stem cell, macrophage, monocyte, or dendritic cell) comprising a CAR as described herein on an unmodified cell (e.g., an immune cell that doesn't comprise a CAR as described herein) can be tested by co-culturing modified immune cells with unmodified immune cells and using flow cytometry to analyze the expression of pro-inflammatory and anti-inflammatory markers in the unmodified cells. In some embodiments, modified immune cells and unmodified immune cells can be co-cultured in a culture dish where the modified immune cells and unmodified immune cells contact each other. In some embodiments, modified immune cells and unmodified immune cells can be co-cultured in a culture dish where the modified immune cells and unmodified immune cells are separated by a transwell assay membrane.

In some embodiments, a modified immune cell (e.g., a stem cell, macrophage, monocyte, or dendritic cell) comprising a CAR as described herein has minimal cytotoxic effects on neighboring cells. In some embodiments, a modified immune cell (e.g., a stem cell, macrophage, monocyte, or dendritic cell) comprising a CAR as described herein has significant cytotoxic effects on neighboring cells (e.g., cancer cells). In some embodiments, modifying an immune cell to comprise a CAR as described herein is not cytotoxic to the modified immune cell. In some embodiments, RNAseq data from modified immune cells are examined to determine if upregulation of genes indicative of cytotoxic effects is present.

In some embodiments, expression of a membrane-tethered cytokine in a modified immune cell (e.g., a stem cell, macrophage, monocyte, or dendritic cell) also comprising a CAR does not decrease a targeted effector function (e.g., phagocytosis, targeted cellular cytotoxicity, antigen presentation, or cytokine secretion) of the modified immune cell relative to a modified immune cell comprising the CAR but not comprising the membrane-tethered cytokine. In some embodiments, expression of a membrane-tethered cytokine in a modified immune cell (e.g., a stem cell, macrophage, monocyte, or dendritic cell) also comprising a CAR increases a targeted effector function (e.g., phagocytosis, targeted cellular cytotoxicity, antigen presentation, or cytokine secretion) of the modified immune cell relative to a modified immune cell comprising the CAR but not comprising the membrane-tethered cytokine.

In some embodiments, a modified immune cell (e.g., a stem cell, macrophage, monocyte, or dendritic cell) comprising a CAR as described herein and/or a membrane-tethered cytokine as described herein may comprise one or more control systems including, but not limited to: a safety switch (e.g., an on switch, an off switch, a suicide switch), transcriptional control (e.g. cell-specific promoters, cell-state specific promoters, promoters downstream of CAR or membrane-tethered cytokine activation, promoters downstream of endogenous signaling pathways, or drug-inducible transcription), post-transcriptional control of CAR or membrane-tethered cytokine mRNA (e.g. RNA-based inhibition with endogenous or recombinant miRNA), or post-translational control of a CAR or membrane-tethered cytokine's structure or stability (e.g. a CAR or membrane-tethered cytokine whose intracellular domain conditionally associates with the full structure by drug/light-inducible association (to allow signaling) or dissociation (to inhibit signaling), or whose stability is drug-regulated for inducible stabilization (to allow signaling) or degradation (to inhibit signaling)). These control systems can be combined to create logic gates, for example an AND gate (e.g. a CAR or membrane-tethered cytokine with a CAR or membrane-tethered cytokine-inducible promoter and cytosolic domain that associates in a drug-dependent manner, thus requiring CAR or membrane-tethered cytokine activation and the presence of a small molecule), an OR gate (e.g. a CAR or membrane-tethered cytokine under control of a promoter that is transcriptionally active following CAR or membrane-tethered activation or small molecule addition), and/or a NOT gate (e.g. a CAR or membrane-tethered cytokine whose mRNA is degraded by endogenous miRNAs expressed in natural immune cell signaling states (such as miRNAs upregulated by a particular cytokine signaling pathway, thus only expressing CAR or membrane-tethered cytokine in the absence of this cytokine)).

### Assays

A variety of assays may be performed to confirm the presence of a nucleic acid construct as described herein and/or the presence of a protein (e.g., a CAR and/or a membrane-tethered cytokine) in an immune cell (e.g., a stem cell, macrophage, monocyte, or dendritic cell). For example, such assays include molecular biological assays well known to those of skill in the art, such as Southern and Northern blotting, RT-PCR, and PCR; and biochemical assays, such as detecting the presence or absence of a particular peptide, e.g., by immunological means (ELISAs and Western blots). Other assays of the present disclosure, include, for example, fluorescence-activated cell sorting (FACS), immunofluorescent microscopy, MSD cytokine analysis, mass spectrometry (MS), RNA-Seq and functional assays.

A variety of assays may be performed to determine various characteristics of a modified immune cell (e.g., a stem cell, macrophage, monocyte, or dendritic cell), such as, but not limited to, immune cell viability, nucleic acid expression, nucleic acid longevity, protein (e.g., CAR and/or membrane-tethered cytokine) expression, protein (e.g., CAR and/or membrane-tethered cytokine) longevity, effector activity, and pro-inflammatory (M1) polarization. For example, such assays include flow cytometry, quantitative PCR, and in vitro functional assays such as cytokine/chemokine secretion, phagocytosis, and specific lysis assays of target tumor cells.

### Chimeric Antigen Receptors (CAR)

The term “chimeric antigen receptor” or “CAR,” as used herein, refers to an artificial cell surface receptor that is engineered to be expressed on an immune effector cell and specifically targets a cell and/or binds an antigen. CARs may be used, for example, as a therapy with adoptive cell transfer. For example, in some embodiments, immune cells (e.g., stem cells, macrophages, monocytes, and/or dendritic cells) are removed from a patient (e.g., from blood, tumor or ascites fluid) and modified so that they express a receptor specific to a particular form of antigen. In some embodiments, such modified immune cells are then reintroduced to the same or a different subject as a therapeutics. In some embodiments, CARs have been expressed with specificity to an antigen, for example, a tumor associated antigen. In some embodiments, a CAR comprises an extracellular domain, a transmembrane domain and one or more intracellular moieties.

In some embodiments, a modified immune cell, for example, a modified stem cell, macrophage, monocyte, or dendritic cell, is generated by expressing a CAR therein. In some embodiments, an immune cell comprises a CAR comprising an extracellular domain, a transmembrane domain, and one or more intracellular moieties, wherein the immune cell comprises a stem cell, macrophage, monocyte, or dendritic cell.

In some embodiments, a CAR may further comprise one or more of: one or more extracellular leader domains, one or more extracellular hinge domains and one or more intracellular co-stimulatory domains.

In some embodiments, a CAR comprises a spacer domain or hinge between an extracellular domain and a transmembrane domain. In some embodiments, a CAR comprises a spacer domain or hinge between one or more intracellular moieties and a transmembrane domain. As used herein, the term “spacer domain” or “hinge” refers to any oligo- or polypeptide that functions to link a transmembrane domain to either an extracellular domain or to one or more intracellular moieties in a polypeptide chain. In some embodiments, a spacer domain or hinge may comprise up to 300 amino acids, preferably 10 to 100 amino acids and most preferably 25 to 50 amino acids. In some embodiments, a short oligo- or polypeptide linker, preferably between 2 and 10 amino acids in length, may form a linkage between a transmembrane domain and one or more intracellular moieties of a CAR. An example of a linker includes a glycine-serine doublet.

In some embodiments, an immune cell (e.g., a stem cell, macrophage, monocyte, or dendritic cell) comprising a CAR may comprise one or more control systems including, but not limited to: a safety switch (e.g., an on switch, and off switch, a suicide switch), a logic gate, for example an AND gate (e.g., two or more CARs, each of which lacks one or more signaling domains such that activation of both/all CARs is required for full immune cell (e.g., stem cell, macrophage, monocyte, or dendritic cell) activation or function), an OR gate (e.g., two or more CARs, each with an intracellular domain such as CD35 and a co-stimulatory domain), and/or a NOT gate (e.g., two or more CARs, one of which includes an inhibitory domain that antagonizes the function of the other CAR[s]).

The present disclosure also provides immune cells (e.g., stem cells, macrophages, monocytes, or dendritic cells) comprising a nucleic acid sequence (e.g., an isolated nucleic acid sequence) encoding a CAR, wherein the nucleic acid sequence comprises a nucleic acid sequence encoding an extracellular domain, a nucleic acid sequence encoding a transmembrane domain and a nucleic acid sequence encoding one or more intracellular moieties, wherein the cell is a stem cell, macrophage, monocyte or dendritic cell that expresses the CAR.

In some embodiments, a CAR comprises an extracellular domain that is operably linked to another domain of the CAR, such as a transmembrane domain or one or more intracellular moieties, for expression in an immune cell. In some embodiments, a nucleic acid encoding an extracellular domain is operably linked to a nucleic acid encoding a transmembrane domain and the nucleic acid encoding the transmembrane domain is operably linked to a nucleic acid encoding one or more intracellular moieties.

In some embodiments, an effector activity of an immune cell comprising a CAR is directed against a target cell comprising an antigen that specifically binds an antigen binding domain of the CAR. In some embodiments, a targeted effector activity directed against a target cell is or comprises phagocytosis, targeted cellular cytotoxicity, antigen presentation, or cytokine secretion.

In some embodiments, a CAR described herein comprises at least one domain (e.g., an extracellular domain, a transmembrane domain, and/or an intracellular domain) that inhibits anti-phagocytic signaling in an immune cell described herein (e.g., a stem cell, macrophage, monocyte, or dendritic cell). In some embodiments, a CAR described herein improves effector activity of an immune cell described herein (e.g., a stem cell, macrophage, monocyte, or dendritic cell), e.g., by enhancing inhibition of CD47 and/or SIRPa activity. In some embodiments, a CAR described herein binds CD47, e.g., and serves as a dominant negative receptor, inhibiting SIRPa activity (e.g., a CD47 sink). In some embodiments, a CAR described herein that binds SIRPa, e.g., comprises an activating receptor (e.g., comprises a CD3z intracellular domain). In some embodiments, a CAR described herein inhibits at least one interaction of CD47 and SIRPa. In some embodiments, a CAR is or comprises a phagocytic logic gate.

In some embodiments, an immune cell described herein (e.g., comprising or expressing a CAR as described herein) comprises or expresses at least one variant or fragment of: SIRPα (e.g., a dominant negative SIRPα or a high-affinity engineered variant of SIRPα (e.g., CV1)), 5F9 scFv, B6H12 scFv (e.g., a humanized B6H12 scFv), PD1 (e.g., a dominant negative PD1 or HAC-I), anti-PD1 scFv (e.g., E27 or durvalumab), Siglec-10, Siglec-9, Siglec-11, and/or SHP-1. In some embodiments, a variant or fragment comprises a mutated intracellular domain. In some embodiments, a variant or fragment does not comprise or express at least one intracellular domain (e.g., an immune cell comprises or expresses an anti-CD47 scFv, CD8a hinge domain, and CD8a transmembrane domain). In some embodiments, an immune cell described herein (e.g., comprising or expressing a CAR as described herein) comprises a dominant negative receptor, e.g., blocking an inhibitory checkpoint.

In some embodiments, a CAR described herein further comprises a cleavage peptide (e.g., a P2A, F2A, E2A and/or T2A peptide) and at least one of a second CAR comprising at least one inhibitory domain of anti-phagocytic signaling and/or a membrane-tethered cytokine as described herein. In some embodiments, at least one second CAR comprises a SIRPα (e.g., a high-affinity engineered variant of SIRPα (e.g., CV1)), 5F9 scFv, B6H12 scFv (e.g., a humanized B6H12 scFv), or a CD47 binding extracellular domain or a fragment thereof. In some embodiments, at least one second CAR comprises a SIRPα transmembrane domain or a fragment thereof. In certain embodiments, a second CAR further comprises a hinge domain (e.g., a CD8a hinge domain). In certain embodiments, at least one second CAR comprises: (i) a leader sequence (e.g., a CD8a leader); ii) an extracellular domain (e.g., a SIRPa, CV1, 5F9 scFv, or B6H12 scFv (e.g., a humanized B6H12 scFv) extracellular domain); and ii) a transmembrane domain (e.g., a SIRPα transmembrane domain). In some embodiments, a CAR described herein further comprises a cleavage peptide (e.g., a P2A peptide) and at least one marker protein (e.g., CD20 or a fragment thereof, CD19 or a fragment thereof, NGFR or a fragment thereof, a synthetic peptide, and/or a fluorescent protein).

In some embodiments, an immune cell described herein (e.g., comprising or expressing a CAR as described herein) comprises or expresses one or more phosphatase dead domains (e.g. a phosphatase dead Shp1, phosphatase dead 72-5ptase (INPP5E), phosphatase dead Shp2, and/or phosphatase dead SHIP-1 domain) and/or a constitutively active kinase domain (e.g., a constitutively active LYN domain). In some embodiments, a CAR described herein further comprises a cleavage peptide (e.g., a P2A, F2A, E2A and/or T2A peptide) and one or more phosphatase dead domains (e.g. a phosphatase dead Shp1, phosphatase dead 72-5ptase (INPP5E), phosphatase dead Shp2, and/or phosphatase dead SHIP-1 domain) and/or a constitutively active kinase domain (e.g., a constitutively active LYN domain).

In some embodiments, a CAR of the present disclosure comprises an amino acid sequence at least 80% identical to a sequence selected from Table 2. In some embodiments, a CAR of the present disclosure comprises an amino acid sequence at least 85% identical to a sequence selected from Table 2. In some embodiments, a CAR of the present disclosure comprises an amino acid sequence at least 90% identical to a sequence selected from Table 2. In some embodiments, a CAR of the present disclosure comprises an amino acid sequence at least 95% identical to a sequence selected from Table 2. In some embodiments, a CAR of the present disclosure comprises an amino acid sequence at least 96% identical to a sequence selected from Table 2. In some embodiments, a CAR of the present disclosure comprises an amino acid sequence at least 97% identical to a sequence selected from Table 2. In some embodiments, a CAR of the present disclosure comprises an amino acid sequence at least 98% identical to a sequence selected from Table 2. In some embodiments, a CAR of the present disclosure comprises an amino acid sequence at least 99% identical to a sequence selected from Table 2. In some embodiments, a CAR of the present disclosure comprises an amino acid sequence identical to a sequence selected from Table 2.

In some embodiments, a CAR of the present disclosure is encoded by a nucleic acid sequence at least 80% identical to a sequence selected from Table 3. In some embodiments, a CAR of the present disclosure is encoded by a nucleic acid sequence at least 85% identical to a sequence selected from Table 3. In some embodiments, a CAR of the present disclosure is encoded by a nucleic acid sequence at least 90% identical to a sequence selected from Table 3. In some embodiments, a CAR of the present disclosure is encoded by a nucleic acid sequence at least 95% identical to a sequence selected from Table 3. In some embodiments, a CAR of the present disclosure is encoded by a nucleic acid sequence at least 96% identical to a sequence selected from Table 3. In some embodiments, a CAR of the present disclosure is encoded by a nucleic acid sequence at least 97% identical to a sequence selected from Table 3. In some embodiments, a CAR of the present disclosure is encoded by a nucleic acid sequence at least 98% identical to a sequence selected from Table 3. In some embodiments, a CAR of the present disclosure is encoded by a nucleic acid sequence at least 99% identical to a sequence selected from Table 3. In some embodiments, a CAR of the present disclosure is encoded by a nucleic acid sequence identical to a sequence selected from Table 3.

### CAR Extracellular Domains

The present disclosure provides chimeric antigen receptors (CAR) comprising one or more extracellular domains. In some embodiments, a CAR extracellular domain comprises an Fc receptor (FcR) extracellular domain. In some embodiments, a CAR extracellular domain comprises a toll-like receptor (TLR) extracellular domain. In some embodiments, a CAR extracellular domain comprises a leader domain. In some embodiments, a CAR extracellular domain comprises an antigen binding domain. In some embodiments, a CAR extracellular domain comprises a hinge domain. In some embodiments, a CAR extracellular domain comprises one or more of an FcR extracellular domain, a TLR extracellular domain, a leader domain, an antigen binding domain and a hinge domain. In some embodiments, a CAR extracellular domain may be a domain that is endogenous to a particular immune cell type (e.g., a modified immune cell as provided herein). In some embodiments, a CAR extracellular domain may be a domain that is not endogenous to a particular immune cell type (e.g., a modified immune cell as provided herein).

### FcR Extracellular Domains

In some embodiments, an FcR extracellular domain comprises a full-length FcR extracellular domain. In some embodiments, an FcR extracellular domain comprises a portion of a full-length FcR extracellular domain. In some embodiments, an FcR extracellular domain (or portion thereof) is or comprises a human FcR extracellular domain. In some embodiments, an FcR extracellular domain may be a domain that is endogenous to a particular immune cell type (e.g., a modified immune cell as provided herein). In some embodiments, an FcR extracellular domain may be a domain that is not endogenous to a particular immune cell type (e.g., a modified immune cell as provided herein). In some embodiments, an FcR extracellular domain comprises a CD64 (FcγRI), CD32a (FcγRIIa), CD32b (FcγRIIb), CD32c, CD16a (FcγRIIIa), CD16b (FcγRIIIb), FcERI, FcERII, or FcaRI (CD89) domain.

### TLR Extracellular Domains

In some embodiments, a TLR extracellular domain comprises a full-length TLR extracellular domain. In some embodiments, a TLR extracellular domain comprises a portion of a full-length TLR extracellular domain. In some embodiments, a TLR extracellular domain (or portion thereof) is or comprises a human TLR extracellular domain. In some embodiments, a TLR extracellular domain may be a domain that is endogenous to a particular immune cell type (e.g., a modified immune cell as provided herein). In some embodiments, a TLR extracellular domain may be a domain that is not endogenous to a particular immune cell type (e.g., a modified immune cell as provided herein). In some embodiments, a TLR extracellular domain comprises a TLR1, TLR2, TLR3, TLR4, TLR5, TLR6, TLR7, TLR8, or TLR9 domain.

### Leader Domains

In some embodiments, a CAR comprises one or more extracellular leader domains. In some embodiments, a nucleic acid encoding a CAR comprises a nucleic acid sequence encoding an extracellular leader domain, but the extracellular leader domain is cleaved from the CAR before the CAR is expressed in an immune cell. In some embodiments, an extracellular leader domain is or comprises a human extracellular leader domain. In some embodiments, an extracellular leader domain may be a domain that is endogenous to a particular immune cell type (e.g., a modified immune cell as provided herein). In some embodiments, an extracellular leader domain may be a domain that is not endogenous to a particular immune cell type (e.g., a modified immune cell as provided herein). In some embodiments, an extracellular leader domain comprises a CD8a extracellular leader domain. In some embodiments, an extracellular leader domain comprises a leader domain from a stimulatory or co-stimulatory domain (e.g., a TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88 domain).

### Antigen Binding Domains

In some embodiments, a CAR comprises at least one antigen binding domain that binds to an antigen, for example, on a target cell. In some embodiments, a CAR comprises an antigen binding domain that binds to an antigen associated with viral infection, bacterial infection, parasitic infection, autoimmune disease, and/or cancer cells. In some embodiments, a CAR antigen binding domain recognizes an antigen that acts as a cell surface marker on a target cell associated with a particular disease state.

In some embodiments, a CAR antigen binding domain binds to a tumor antigen, such as an antigen that is specific for a tumor or cancer of interest. In some embodiments a tumor antigen comprises one or more antigenic cancer epitopes. In some embodiments, a tumor antigen comprises CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1). In certain embodiments, a tumor antigen comprises ERBB2 (Her2/neu). In certain embodiments, a tumor antigen comprises PSMA. In certain embodiments, a tumor antigen comprises Mesothelin.

In some embodiments, an antigen binding domain binds to a misfolded protein antigen or a protein of a protein aggregate, such as a protein that is specific for a disease/disorder of interest. In some embodiments, the disease/disorder is a neurodegenerative disease/disorder, an inflammatory disease/disorder, a cardiovascular disease/disorder, a fibrotic disease/disorder, or amyloidosis (e.g., mediated by protein aggregates of immunoglobulin light chains or of transthyretin). In some embodiments, the neurodegenerative disease/disorder is selected from the group consisting of tauopathy, asynucleopathy, presenile dementia, senile dementia, Alzheimer's disease (mediated by protein aggregates ofbeta-amyloid), Parkinsonism linked to chromosome 17 (FTDP-17), progressive supranuclear palsy (PSP), Pick's disease, primary progressive aphasia, frontotemporal dementia, corticobasal dementia, Parkinson's disease, Parkinson's disease with dementia, dementia with Lewy bodies, Down syndrome, multiple system atrophy, amyotrophic lateral sclerosis (ALS), Hallervorden-Spatz syndrome, polyglutamine disease, trinucleotide repeat disease, Familial British dementia, Fatal Familial Insomnia, Gerstmann-Straussler-Scheinker Syndrome, Hereditary cerebral hemorrhage with amyloidosis (Icelandic) (HCHW A-I), Sporadic Fatal Insomnia (sFI), Variably Protease-Sensitive Prionopathy (VPSPr), Familial Danish dementia, and prion disease (such as Creutzfeldt-Jakob disease, CJD and Variant Creutzfeldt-Jakob Disease (vCJD)).

In some embodiments, an antigen binding domain comprises any domain that binds to an antigen. In some embodiments, a CAR antigen binding domain is or comprises a monoclonal antibody, a polyclonal antibody, a synthetic antibody, a human antibody, a humanized antibody, a non-human antibody, or any fragment thereof, for example an scFv. In some embodiments, a CAR antigen binding domain is or comprises an aptamer, a darpin, a centyrin, a naturally occurring or synthetic receptor, an affibody, or other engineered protein recognition molecule. In some embodiments, a CAR antigen binding domain is or comprises a mammalian antibody or a fragment thereof. In some embodiments, a CAR antigen binding domain is derived, in whole or in part, from the same species in which the CAR will ultimately be used. For example, for use in humans, an antigen binding domain of a CAR comprises a human antibody, a humanized antibody, or a fragment thereof (e.g. a scFv). In some embodiments, a CAR antigen binding domain may be a domain that is endogenous to a particular immune cell type (e.g., a modified immune cell as provided herein). In some embodiments, a CAR antigen binding domain may be a domain that is not endogenous to a particular immune cell type (e.g., a modified immune cell as provided herein).

In some embodiments, a CAR comprises one or more antigen binding domains. In some embodiments, a CAR comprises two or more antigen binding domains. In some embodiments, a CAR is a bispecific CAR. In some embodiments, an immune cell comprises two or more different CARs comprising one or more antigen binding domains. In some embodiments, an immune cell comprising a bispecific CAR and/or comprising two or more different CARs comprising one or more antigen binding domains can reduce off-target and/or on-target off-tissue effects by requiring that two antigens are present. In some embodiments, an immune cell comprises a bispecific CAR and/or comprises two or more different CARS comprising one or more antigen binding domains, wherein the CARs provide distinct signals that in isolation are insufficient to mediate activation of the modified cell, but are synergistic together, stimulating activation of the modified cell. In some embodiments, such a construct may be referred to as an ‘AND’ logic gate.

In some embodiments, an immune cell comprising a bispecific CAR and/or comprising two or more different CARs comprising one or more antigen binding domains can reduce off-target and/or on-target off-tissue effects by requiring that one antigen is present and a second, normal protein antigen is absent before the cell's activity is stimulated. In some embodiments, such a construct may be referred to as a ‘NOT’ logic gate. In contrast to AND gates, NOT gated CAR-modified cells are activated by binding to a single antigen. However, the binding of a second receptor to the second antigen functions to override the activating signal being perpetuated through the CAR. Typically, such an inhibitory receptor would be targeted against an antigen that is abundantly expressed in a normal tissue but is absent in tumor tissue.

### Hinge Domains

In some embodiments, a CAR comprises one or more extracellular hinge domains. In some embodiments, a CAR extracellular hinge domain is or comprises a human extracellular hinge domain. In some embodiments, a CAR extracellular hinge domain may be a domain that is endogenous to a particular immune cell type (e.g., a modified immune cell as provided herein). In some embodiments, a CAR extracellular hinge domain may be a domain that is not endogenous to a particular immune cell type (e.g., a modified immune cell as provided herein). In some embodiments, one or more CAR extracellular hinge domains comprise a CD8a extracellular hinge domain or an IgG4 or a CD28 extracellular hinge domain. In some embodiments, a CAR comprises or includes a CD28 hinge domain. In some embodiments, a CAR comprises or includes a CD8 hinge domain. In some embodiments, a CAR extracellular hinge domain optimizes the physicochemical parameters of a CAR, e.g., optimal size relative to tumor antigen (e.g., allowing for exclusion of inhibitory molecules), optimal flexibility, optimal protein folding, optimal protein stability, optimal binding, optimal homodimerization, and/or lack of homodimerization.

### CAR Transmembrane Domains

In some embodiments, a CAR comprises a transmembrane domain, for example, that connects an extracellular domain to one or more intracellular moieties. In some embodiments, a CAR transmembrane domain is naturally associated with one or more other domain(s) of a CAR. In some embodiments, a CAR transmembrane domain can be modified to avoid binding to transmembrane domains of other surface membrane proteins, in order to minimize interactions with other members of a receptor complex. In some embodiments, a CAR transmembrane domain may be derived either from a naturally-occurring or from a synthetic source. In some embodiments a CAR transmembrane domain is derived from a naturally-occurring membrane-bound or transmembrane protein. In some embodiments, a CAR transmembrane domain is or comprises a human transmembrane domain. In some embodiments, a CAR transmembrane domain may be a domain that is endogenous to a particular immune cell type (e.g., a modified immune cell as provided herein). In some embodiments, a CAR transmembrane domain may be a domain that is not endogenous to a particular immune cell type (e.g., a modified immune cell as provided herein). In some embodiments, a CAR transmembrane domain comprises a CD8a, CD64, CD32a, CD32c, CD16a, TRL1, TLR2, TLR3, TRL4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88, CD3-zeta, FCR Y, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPB, CD22, PIR-B, LILRB1, CD36, or Syk transmembrane domain. In some embodiments, a CAR comprises or includes a CD28 transmembrane domain. In some embodiments, a CAR comprises or includes a CD8 transmembrane domain.

### FcR Transmembrane Domains

In some embodiments, an FcR transmembrane domain comprises a full-length FcR transmembrane domain. In some embodiments, an FcR transmembrane domain comprises a portion of a full-length FcR transmembrane domain. In some embodiments, an FcR transmembrane domain is or comprises a human FcR transmembrane domain, or portion thereof. In some embodiments, an FcR transmembrane domain may be a domain that is endogenous to a particular immune cell type (e.g., a modified immune cell as provided herein). In some embodiments, an FcR transmembrane domain may be a domain that is not endogenous to a particular immune cell type (e.g., a modified immune cell as provided herein). In some embodiments, an FcR transmembrane domain comprises a CD64 (FcγRI), CD32a (FcγRIIa), CD32b (FcγRIIb), CD32c, CD16a (FcγRIIIa), CD16b (FcγRIIIb), FcERI, FcERII, or FcaRI (CD89) domain.

### TLR Transmembrane Domains

In some embodiments, a TLR transmembrane domain comprises a full-length TLR transmembrane domain. In some embodiments, a TLR transmembrane domain comprises a portion of a full-length TLR transmembrane domain. In some embodiments, a TLR transmembrane domain is or comprises a human TLR transmembrane domain, or portion thereof. In some embodiments, a TLR transmembrane domain may be a domain that is endogenous to a particular immune cell type (e.g., a modified immune cell as provided herein). In some embodiments, a TLR transmembrane domain may be a domain that is not endogenous to a particular immune cell type (e.g., a modified immune cell as provided herein). In some embodiments, a TLR transmembrane domain comprises a TLR1, TLR2, TLR3, TLR4, TLR5, TLR6, TLR7, TLR8, or TLR9 domain.

### CAR Intracellular Moieties

In some embodiments, a CAR comprises one or more intracellular moieties. In some embodiments, an intracellular moiety is or comprises a human intracellular moiety, or portion thereof. In some embodiments, an intracellular moiety may be or comprise a domain that is endogenous to a particular immune cell type (e.g., a modified immune cell as provided herein). In some embodiments, an intracellular moiety may be or comprise a domain that is not endogenous to a particular immune cell type (e.g., a modified immune cell as provided herein). In some embodiments, an intracellular moiety and/or other cytoplasmic moiety of a CAR is responsible for activation of the cell in which the CAR is expressed (e.g., an immune cell). In some embodiments, an intracellular moiety of a CAR is responsible for signal activation and/or transduction in an immune cell comprising said CAR.

### JAK Binding Domains and STAT Binding Domains

In some embodiments, an intracellular moiety is or comprises a first JAK binding domain or a portion thereof. In some embodiments, an intracellular moiety is or comprises a first STAT binding domain or a portion thereof. In some embodiments, an intracellular moiety is or comprises a first JAK binding domain or portion thereof and a first STAT binding domain or portion thereof. In some embodiments, an intracellular moiety is or comprises a second JAK binding domain or a portion thereof. In some embodiments, an intracellular moiety is or comprises a second STAT binding domain or a portion thereof. In some embodiments, an intracellular moiety is or comprises a first intracellular domain or a portion thereof. In some embodiments, an intracellular moiety is or comprises a second intracellular domain or a portion thereof.

In some embodiments, one or more intracellular moieties are, in order (from cellular membrane proximal to distal): (i) a first JAK binding domain or portion thereof, a first STAT binding domain or portion thereof, and a first intracellular domain or portion thereof; (ii) a first STAT binding domain or portion thereof, a first JAK binding domain or portion thereof, and a first intracellular domain or portion thereof; (iii) a first JAK binding domain or portion thereof, a first intracellular domain or portion thereof, and a first STAT binding domain or portion thereof; (iv) a first STAT binding domain or portion thereof, a first intracellular domain or portion thereof, and a first JAK binding domain or portion thereof; (v) a first intracellular domain or portion thereof, a first JAK binding domain or portion thereof, and a first STAT binding domain or portion thereof; (vi) a first intracellular domain or portion thereof, a first STAT binding domain or portion thereof, and a first JAK binding domain or portion thereof; (vii) a first JAK binding domain or portion thereof and a first STAT binding domain or portion thereof; or (viii) a first STAT binding domain or portion thereof and a first JAK binding domain or portion thereof.

In some embodiments, one or more intracellular moieties are, in order (from cellular membrane proximal to distal): a first JAK binding domain or portion thereof, a first STAT binding domain or portion thereof, a first intracellular domain or portion thereof, and a second STAT binding domain or portion thereof. In some embodiments, one or more intracellular moieties are, in order (from cellular membrane proximal to distal): a first intracellular domain or portion thereof, a first JAK binding domain or portion thereof, a first STAT binding domain or portion thereof, a spacer, and a second STAT binding domain or portion thereof. In some embodiments, one or more intracellular moieties are, in order (from cellular membrane proximal to distal): a first intracellular domain or portion thereof, a first JAK binding domain or portion thereof, a first STAT binding domain or portion thereof, a spacer, a second STAT binding domain or portion thereof, a spacer, and a third STAT binding domain or portion thereof.

In some embodiments, a modified immune cell (e.g., a stem cell, macrophage, monocyte, or dendritic cell) of the present disclosure comprises a CAR comprising one or more intracellular moieties comprising one, two, three, four, five or six of: a first JAK binding domain or portion thereof, a second JAK binding domain or portion thereof, a first STAT binding domain or portion thereof, a second STAT binding domain or portion thereof, a first intracellular domain or portion thereof and/or a second intracellular domain or portion thereof in any order (from cellular membrane proximal to distal). In some embodiments, a modified immune cell (e.g., a stem cell, macrophage, monocyte, or dendritic cell) of the present disclosure comprises a CAR comprising one or more intracellular moieties comprising one, two, three, four, five, six, or seven of: a first JAK binding domain or portion thereof, a second JAK binding domain or portion thereof, a first STAT binding domain or portion thereof, a second STAT binding domain or portion thereof, a third STAT binding domain or portion thereof, a first intracellular domain or portion thereof and/or a second intracellular domain or portion thereof in any order (from cellular membrane proximal to distal).

In some embodiments, the order of the one or more intracellular moieties (from cellular membrane proximal to distal) in the CAR architecture directly influences their function. In some embodiments, a CAR comprising one or more JAK and/or STAT binding domains membrane proximal (relative to one or more intracellular domains, for example, an ITAM domain or portion thereof) exhibits constitutive STAT signaling. In some embodiments, a CAR comprising one or more JAK and/or STAT binding domains membrane proximal (relative to one or more intracellular domains, for example, an ITAM domain or portion thereof) exhibits inducible intracellular domain signaling (e.g., induced via binding of a target to an extracellular portion of the CAR). In some embodiments, a modified immune cell (e.g., a stem cell, macrophage, monocyte, or dendritic cell) of the present disclosure comprising a CAR comprising one or more JAK and/or STAT binding domains membrane proximal (relative to one or more intracellular domains, for example, an ITAM domain or portion thereof) exhibits polarization toward an inflammatory (e.g., M1) or anti-inflammatory (e.g., M2) state and/or secretion of specific cytokines (based on the one or more JAK and/or STAT binding domains) in the absence of the CAR binding a target antigen.

In some embodiments, a CAR comprising one or more JAK and/or STAT binding domains membrane distal (relative to one or more intracellular domains, for example, an ITAM domain or portion thereof) exhibits inducible STAT signaling. In some embodiments, a CAR comprising one or more JAK and/or STAT binding domains membrane distal (relative to one or more intracellular domains, for example, an ITAM domain or portion thereof) exhibits constitutive intracellular domain signaling. In some embodiments, a modified immune cell (e.g., a stem cell, macrophage, monocyte, or dendritic cell) of the present disclosure comprising a CAR comprising one or more JAK and/or STAT binding domains membrane distal (relative to one or more intracellular domains, for example, an ITAM domain or portion thereof) exhibits polarization toward an inflammatory (e.g., M1) or anti-inflammatory (e.g., M2) state and/or secretion of specific cytokines (based on the one or more JAK and/or STAT binding domains) upon binding of the CAR binding a target antigen.

In some embodiments, a first JAK binding domain or portion thereof is derived from a first cytokine receptor. In some embodiments, a first STAT binding domain or portion thereof is derived from a second cytokine receptor. In some embodiments, a second JAK binding domain or portion thereof is derived from a third cytokine receptor. In some embodiments, a second STAT binding domain or portion thereof is derived from a fourth cytokine receptor. In some embodiments, a third STAT binding domain or portion thereof is derived from a first, second, third, or fourth cytokine receptor. In some embodiments, a first cytokine receptor and a second cytokine receptor are different cytokine receptors (e.g., different types of cytokine receptors, e.g., a first cytokine receptor comprises a G-CSF receptor or portion thereof and a second cytokine receptor comprises an IFNγ receptor or portion thereof). In some embodiments, a first cytokine receptor and a second cytokine receptor are the same cytokine receptor (e.g., the same type of cytokine receptor, e.g., an IL-10 receptor or portion thereof). In some embodiments, a third cytokine receptor and a fourth cytokine receptor are different cytokine receptors. In some embodiments, a third cytokine receptor and a fourth cytokine receptor are the same cytokine receptor. In some embodiments, a first cytokine receptor and a third cytokine receptor are different cytokine receptors. In some embodiments, a first cytokine receptor and a third cytokine receptor are the same cytokine receptor. In some embodiments, a second cytokine receptor and a fourth cytokine receptor are different cytokine receptors. In some embodiments, a second cytokine receptor and a fourth cytokine receptor are the same cytokine receptor.

In some embodiments, a first cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL 7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a second cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a third cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL 7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a fourth cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

In some embodiments, a first JAK binding domain or portion thereof binds JAK1, JAK2, JAK3, TYK2, or combinations thereof. In some embodiments, a second JAK binding domain or portion thereof binds JAK1, JAK2, JAK3, TYK2, or combinations thereof. In some embodiments, a first STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof. In some embodiments, a second STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof.

In some embodiments, a first JAK binding domain or portion thereof is derived from a first cytokine receptor comprising an amino acid sequence at least 80% identical to a sequence selected from Table 4. In some embodiments, a first JAK binding domain or portion thereof is derived from a first cytokine receptor comprising an amino acid sequence at least 85% identical to a sequence selected from Table 4. In some embodiments, a first JAK binding domain or portion thereof is derived from a first cytokine receptor comprising an amino acid sequence at least 90% identical to a sequence selected from Table 4. In some embodiments, a first JAK binding domain or portion thereof is derived from a first cytokine receptor comprising an amino acid sequence at least 95% identical to a sequence selected from Table 4. In some embodiments, a first JAK binding domain or portion thereof is derived from a first cytokine receptor comprising an amino acid sequence at least 96% identical to a sequence selected from Table 4. In some embodiments, a first JAK binding domain or portion thereof is derived from a first cytokine receptor comprising an amino acid sequence at least 97% identical to a sequence selected from Table 4. In some embodiments, a first JAK binding domain or portion thereof is derived from a first cytokine receptor comprising an amino acid sequence at least 98% identical to a sequence selected from Table 4. In some embodiments, a first JAK binding domain or portion thereof is derived from a first cytokine receptor comprising an amino acid sequence at least 99% identical to a sequence selected from Table 4. In some embodiments, a first JAK binding domain or portion thereof is derived from a first cytokine receptor comprising an amino acid sequence identical to a sequence selected from Table 4.

In some embodiments, a first JAK binding domain or portion thereof is derived from a first cytokine receptor encoded by a nucleic acid sequence at least 80% identical to a sequence selected from Table 5. In some embodiments, a first JAK binding domain or portion thereof is derived from a first cytokine receptor encoded by a nucleic acid sequence at least 85% identical to a sequence selected from Table 5. In some embodiments, a first JAK binding domain or portion thereof is derived from a first cytokine receptor encoded by a nucleic acid sequence at least 90% identical to a sequence selected from Table 5. In some embodiments, a first JAK binding domain or portion thereof is derived from a first cytokine receptor encoded by a nucleic acid sequence at least 95% identical to a sequence selected from Table 5. In some embodiments, a first JAK binding domain or portion thereof is derived from a first cytokine receptor encoded by a nucleic acid sequence at least 96% identical to a sequence selected from Table 5. In some embodiments, a first JAK binding domain or portion thereof is derived from a first cytokine receptor encoded by a nucleic acid sequence at least 97% identical to a sequence selected from Table 5. In some embodiments, a first JAK binding domain or portion thereof is derived from a first cytokine receptor encoded by a nucleic acid sequence at least 98% identical to a sequence selected from Table 5. In some embodiments, a first JAK binding domain or portion thereof is derived from a first cytokine receptor encoded by a nucleic acid sequence at least 99% identical to a sequence selected from Table 5. In some embodiments, a first JAK binding domain or portion thereof is derived from a first cytokine receptor encoded by a nucleic acid sequence identical to a sequence selected from Table 5.

In some embodiments, a first STAT binding domain or portion thereof is derived from a second cytokine receptor comprising an amino acid sequence at least 80% identical to a sequence selected from Table 4. In some embodiments, a first STAT binding domain or portion thereof is derived from a second cytokine receptor comprising an amino acid sequence at least 85% identical to a sequence selected from Table 4. In some embodiments, a first STAT binding domain or portion thereof is derived from a second cytokine receptor comprising an amino acid sequence at least 90% identical to a sequence selected from Table 4. In some embodiments, a first STAT binding domain or portion thereof is derived from a second cytokine receptor comprising an amino acid sequence at least 95% identical to a sequence selected from Table 4. In some embodiments, a first STAT binding domain or portion thereof is derived from a second cytokine receptor comprising an amino acid sequence at least 96% identical to a sequence selected from Table 4. In some embodiments, a first STAT binding domain or portion thereof is derived from a second cytokine receptor comprising an amino acid sequence at least 97% identical to a sequence selected from Table 4. In some embodiments, a first STAT binding domain or portion thereof is derived from a second cytokine receptor comprising an amino acid sequence at least 98% identical to a sequence selected from Table 4. In some embodiments, a first STAT binding domain or portion thereof is derived from a second cytokine receptor comprising an amino acid sequence at least 99% identical to a sequence selected from Table 4. In some embodiments, a first STAT binding domain or portion thereof is derived from a second cytokine receptor comprising an amino acid sequence identical to a sequence selected from Table 4.

In some embodiments, a first STAT binding domain or portion thereof is derived from a second cytokine receptor encoded by a nucleic acid sequence at least 80% identical to a sequence selected from Table 5. In some embodiments, a first STAT binding domain or portion thereof is derived from a second cytokine receptor encoded by a nucleic acid sequence at least 85% identical to a sequence selected from Table 5. In some embodiments, a first STAT binding domain or portion thereof is derived from a second cytokine receptor encoded by a nucleic acid sequence at least 90% identical to a sequence selected from Table 5. In some embodiments, a first STAT binding domain or portion thereof is derived from a second cytokine receptor encoded by a nucleic acid sequence at least 95% identical to a sequence selected from Table 5. In some embodiments, a first STAT binding domain or portion thereof is derived from a second cytokine receptor encoded by a nucleic acid sequence at least 96% identical to a sequence selected from Table 5. In some embodiments, a first STAT binding domain or portion thereof is derived from a second cytokine receptor encoded by a nucleic acid sequence at least 97% identical to a sequence selected from Table 5. In some embodiments, a first STAT binding domain or portion thereof is derived from a second cytokine receptor encoded by a nucleic acid sequence at least 98% identical to a sequence selected from Table 5. In some embodiments, a first STAT binding domain or portion thereof is derived from a second cytokine receptor encoded by a nucleic acid sequence at least 99% identical to a sequence selected from Table 5. In some embodiments, a first STAT binding domain or portion thereof is derived from a second cytokine receptor encoded by a nucleic acid sequence identical to a sequence selected from Table 5.

In some embodiments, a second JAK binding domain or portion thereof is derived from a third cytokine receptor comprising an amino acid sequence at least 80% identical to a sequence selected from Table 4. In some embodiments, a second JAK binding domain or portion thereof is derived from a third cytokine receptor comprising an amino acid sequence at least 85% identical to a sequence selected from Table 4. In some embodiments, a second JAK binding domain or portion thereof is derived from a third cytokine receptor comprising an amino acid sequence at least 90% identical to a sequence selected from Table 4. In some embodiments, a second JAK binding domain or portion thereof is derived from a third cytokine receptor comprising an amino acid sequence at least 95% identical to a sequence selected from Table 4. In some embodiments, a second JAK binding domain or portion thereof is derived from a third cytokine receptor comprising an amino acid sequence at least 96% identical to a sequence selected from Table 4. In some embodiments, a second JAK binding domain or portion thereof is derived from a third cytokine receptor comprising an amino acid sequence at least 97% identical to a sequence selected from Table 4. In some embodiments, a second JAK binding domain or portion thereof is derived from a third cytokine receptor comprising an amino acid sequence at least 98% identical to a sequence selected from Table 4. In some embodiments, a second JAK binding domain or portion thereof is derived from a third cytokine receptor comprising an amino acid sequence at least 99% identical to a sequence selected from Table 4. In some embodiments, a second JAK binding domain or portion thereof is derived from a third cytokine receptor comprising an amino acid sequence identical to a sequence selected from Table 4.

In some embodiments, a second JAK binding domain or portion thereof is derived from a third cytokine receptor encoded by a nucleic acid sequence at least 80% identical to a sequence selected from Table 5. In some embodiments, a second JAK binding domain or portion thereof is derived from a third cytokine receptor encoded by a nucleic acid sequence at least 85% identical to a sequence selected from Table 5. In some embodiments, a second JAK binding domain or portion thereof is derived from a third cytokine receptor encoded by a nucleic acid sequence at least 90% identical to a sequence selected from Table 5. In some embodiments, a second JAK binding domain or portion thereof is derived from a third cytokine receptor encoded by a nucleic acid sequence at least 95% identical to a sequence selected from Table 5. In some embodiments, a second JAK binding domain or portion thereof is derived from a third cytokine receptor encoded by a nucleic acid sequence at least 96% identical to a sequence selected from Table 5. In some embodiments, a second JAK binding domain or portion thereof is derived from a third cytokine receptor encoded by a nucleic acid sequence at least 97% identical to a sequence selected from Table 5. In some embodiments, a second JAK binding domain or portion thereof is derived from a third cytokine receptor encoded by a nucleic acid sequence at least 98% identical to a sequence selected from Table 5. In some embodiments, a second JAK binding domain or portion thereof is derived from a third cytokine receptor encoded by a nucleic acid sequence at least 99% identical to a sequence selected from Table 5. In some embodiments, a second JAK binding domain or portion thereof is derived from a third cytokine receptor encoded by a nucleic acid sequence identical to a sequence selected from Table 5.

In some embodiments, a second STAT binding domain or portion thereof is derived from a fourth cytokine receptor comprising an amino acid sequence at least 80% identical to a sequence selected from Table 4. In some embodiments, a second STAT binding domain or portion thereof is derived from a fourth cytokine receptor comprising an amino acid sequence at least 85% identical to a sequence selected from Table 4. In some embodiments, a second STAT binding domain or portion thereof is derived from a fourth cytokine receptor comprising an amino acid sequence at least 90% identical to a sequence selected from Table 4. In some embodiments, a second STAT binding domain or portion thereof is derived from a fourth cytokine receptor comprising an amino acid sequence at least 95% identical to a sequence selected from Table 4. In some embodiments, a second STAT binding domain or portion thereof is derived from a fourth cytokine receptor comprising an amino acid sequence at least 96% identical to a sequence selected from Table 4. In some embodiments, a second STAT binding domain or portion thereof is derived from a fourth cytokine receptor comprising an amino acid sequence at least 97% identical to a sequence selected from Table 4. In some embodiments, a second STAT binding domain or portion thereof is derived from a fourth cytokine receptor comprising an amino acid sequence at least 98% identical to a sequence selected from Table 4. In some embodiments, a second STAT binding domain or portion thereof is derived from a fourth cytokine receptor comprising an amino acid sequence at least 99% identical to a sequence selected from Table 4. In some embodiments, a second STAT binding domain or portion thereof is derived from a fourth cytokine receptor comprising an amino acid sequence identical to a sequence selected from Table 4.

In some embodiments, a second STAT binding domain or portion thereof is derived from a fourth cytokine receptor encoded by a nucleic acid sequence at least 80% identical to a sequence selected from Table 5. In some embodiments, a second STAT binding domain or portion thereof is derived from a fourth cytokine receptor encoded by a nucleic acid sequence at least 85% identical to a sequence selected from Table 5. In some embodiments, a second STAT binding domain or portion thereof is derived from a fourth cytokine receptor encoded by a nucleic acid sequence at least 90% identical to a sequence selected from Table 5. In some embodiments, a second STAT binding domain or portion thereof is derived from a fourth cytokine receptor encoded by a nucleic acid sequence at least 95% identical to a sequence selected from Table 5. In some embodiments, a second STAT binding domain or portion thereof is derived from a fourth cytokine receptor encoded by a nucleic acid sequence at least 96% identical to a sequence selected from Table 5. In some embodiments, a second STAT binding domain or portion thereof is derived from a fourth cytokine receptor encoded by a nucleic acid sequence at least 97% identical to a sequence selected from Table 5. In some embodiments, a second STAT binding domain or portion thereof is derived from a fourth cytokine receptor encoded by a nucleic acid sequence at least 98% identical to a sequence selected from Table 5. In some embodiments, a second STAT binding domain or portion thereof is derived from a fourth cytokine receptor encoded by a nucleic acid sequence at least 99% identical to a sequence selected from Table 5. In some embodiments, a second STAT binding domain or portion thereof is derived from a fourth cytokine receptor encoded by a nucleic acid sequence identical to a sequence selected from Table 5.

In some embodiments, a third STAT binding domain or portion thereof is derived from a fourth cytokine receptor comprising an amino acid sequence at least 80% identical to a sequence selected from Table 4. In some embodiments, a third STAT binding domain or portion thereof is derived from a fourth cytokine receptor comprising an amino acid sequence at least 85% identical to a sequence selected from Table 4. In some embodiments, a third STAT binding domain or portion thereof is derived from a fourth cytokine receptor comprising an amino acid sequence at least 90% identical to a sequence selected from Table 4. In some embodiments, a third STAT binding domain or portion thereof is derived from a fourth cytokine receptor comprising an amino acid sequence at least 95% identical to a sequence selected from Table 4. In some embodiments, a third STAT binding domain or portion thereof is derived from a fourth cytokine receptor comprising an amino acid sequence at least 96% identical to a sequence selected from Table 4. In some embodiments, a third STAT binding domain or portion thereof is derived from a fourth cytokine receptor comprising an amino acid sequence at least 97% identical to a sequence selected from Table 4. In some embodiments, a third STAT binding domain or portion thereof is derived from a fourth cytokine receptor comprising an amino acid sequence at least 98% identical to a sequence selected from Table 4. In some embodiments, a third STAT binding domain or portion thereof is derived from a fourth cytokine receptor comprising an amino acid sequence at least 99% identical to a sequence selected from Table 4. In some embodiments, a third STAT binding domain or portion thereof is derived from a fourth cytokine receptor comprising an amino acid sequence identical to a sequence selected from Table 4.

In some embodiments, a third STAT binding domain or portion thereof is derived from a fourth cytokine receptor encoded by a nucleic acid sequence at least 80% identical to a sequence selected from Table 5. In some embodiments, a third STAT binding domain or portion thereof is derived from a fourth cytokine receptor encoded by a nucleic acid sequence at least 85% identical to a sequence selected from Table 5. In some embodiments, a third STAT binding domain or portion thereof is derived from a fourth cytokine receptor encoded by a nucleic acid sequence at least 90% identical to a sequence selected from Table 5. In some embodiments, a third STAT binding domain or portion thereof is derived from a fourth cytokine receptor encoded by a nucleic acid sequence at least 95% identical to a sequence selected from Table 5. In some embodiments, a third STAT binding domain or portion thereof is derived from a fourth cytokine receptor encoded by a nucleic acid sequence at least 96% identical to a sequence selected from Table 5. In some embodiments, a third STAT binding domain or portion thereof is derived from a fourth cytokine receptor encoded by a nucleic acid sequence at least 97% identical to a sequence selected from Table 5. In some embodiments, a third STAT binding domain or portion thereof is derived from a fourth cytokine receptor encoded by a nucleic acid sequence at least 98% identical to a sequence selected from Table 5. In some embodiments, a third STAT binding domain or portion thereof is derived from a fourth cytokine receptor encoded by a nucleic acid sequence at least 99% identical to a sequence selected from Table 5. In some embodiments, a third STAT binding domain or portion thereof is derived from a fourth cytokine receptor encoded by a nucleic acid sequence identical to a sequence selected from Table 5.

### CAR Intracellular Domains

In some embodiments, a CAR comprises one or more intracellular domains. In some embodiments, an intracellular domain is or comprises a human intracellular domain, or portion thereof. In some embodiments, an intracellular domain may be a domain that is endogenous to a particular immune cell type (e.g., a modified immune cell as provided herein). In some embodiments, an intracellular domain may be a domain that is not endogenous to a particular immune cell type (e.g., a modified immune cell as provided herein). In some embodiments, an intracellular domain and/or other cytoplasmic domain of a CAR is responsible for activation of the cell in which the CAR is expressed (e.g., an immune cell). In some embodiments, an intracellular domain of a CAR is responsible for signal activation and/or transduction in an immune cell comprising said CAR.

In some embodiments, an intracellular domain of a CAR includes at least one domain responsible for signal activation and/or transduction. In some embodiments, an intracellular domain is or comprises at least one of a co-stimulatory molecule and a signaling domain. In some embodiments, an intracellular domain of a CAR comprises dual signaling domains. In some embodiments, an intracellular domain of a CAR comprises more than two signaling domains.

In some embodiments, an intracellular domain comprises a cytoplasmic portion of a surface receptor. In some embodiments, an intracellular domain comprises a co-stimulatory molecule. In some embodiments, an intracellular domain comprises a molecule that acts to initiate signal transduction in an immune cell.

In some embodiments, an intracellular domain of a CAR includes any portion of one or more co-stimulatory molecules, such as at least one signaling domain from CD3, Fc epsilon RI gamma chain, any derivative or variant thereof, any synthetic sequence thereof that has the same functional capability, and any combination thereof.

### FcR Intracellular Domains

In some embodiments, an FcR intracellular domain comprises a full-length FcR intracellular domain. In some embodiments, an FcR intracellular domain comprises a portion of a full-length FcR intracellular domain. In some embodiments, an FcR intracellular domain is or comprises a human FcR intracellular domain, or portion thereof. In some embodiments, an FcR intracellular domain may be a domain that is endogenous to a particular immune cell type (e.g., a modified immune cell as provided herein). In some embodiments, an FcR intracellular domain may be a domain that is not endogenous to a particular immune cell type (e.g., a modified immune cell as provided herein). In some embodiments, an FcR intracellular domain comprises a CD64 (FcγRI), CD32a (FcγRIIa), CD32b (FcγRIIb), CD32c, CD16a (FcγRIIIa), CD16b (FcγRIIIb), FcεRI, FcεRII, or FcαRI (CD89) domain.

### TLR Intracellular Domains

In some embodiments, a TLR intracellular domain comprises a full-length TLR intracellular domain. In some embodiments, a TLR intracellular domain comprises a portion of a full-length TLR intracellular domain. In some embodiments, a TLR intracellular domain is or comprises a human TLR intracellular domain, or portion thereof. In some embodiments, a TLR intracellular domain may be a domain that is endogenous to a particular immune cell type (e.g., a modified immune cell as provided herein). In some embodiments, a TLR intracellular domain may be a domain that is not endogenous to a particular immune cell type (e.g., a modified immune cell as provided herein). In some embodiments, a TLR intracellular domain comprises a TLR1, TLR2, TLR3, TLR4, TLR5, TLR6, TLR7, TLR8, or TLR9 domain.

### Signaling Domains

In some embodiments, a CAR comprises one or more intracellular signaling domains. In some embodiments, an intracellular signaling domain is or comprises a human intracellular signaling domain, or portion thereof. In some embodiments, a signaling domain may be a domain that is endogenous to a particular immune cell type (e.g., a modified immune cell as provided herein). In some embodiments, a signaling domain may be a domain that is not endogenous to a particular immune cell type (e.g., a modified immune cell as provided herein).

In some embodiments, one or more intracellular signaling domains may be or comprise a CD3-zeta, FcR γ, CD64, CD32a, CD32c, CD16a, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD40, CD19, CD20, 41BB, CD28, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPβ, CD22, PIR-B, LILRB1, Syk, 41BB ligand (41BBL; TNFSF9), CD27, OX40L, CD32b, CD11b, ITGAM, SLAMF7, CD206, CD163, CD209, Dectin-2, or one or more cytokine receptor signaling domains (e.g., an ILIR, an IL2R, an IL3R, an ILAR, an IL5R, an IL6R, an IL7R, an IL8R, an IL9R, an IL10R, an IL11R, an IL12R, an IL13R, an IL14R, an IL15R, an IL17R, an IFNaR, an IFNgR, an TNFR, an CSFIR, an CSF2R, Dap10, CD36, Dectin-1, or ICOSL intracellular signaling domain).

In some embodiments, an intracellular domain of a CAR may be or comprise dual signaling domains, such as 41BB, CD28, ICOS, TLR1, TLR2, TLR3, TLR4, TLR5, TLR6, TLR7, TLR8, TLR9, TLR10, TLR11, CD116 receptor beta chain, CSF1-R, LRP1/CD91, SR-A1, SR-A2, MARCO, SR-CL1, SR-CL2, SR—C, SR-E, CR1, CR3, CR4, dectin 1, DEC-205, DC-SIGN, CD14, CD36, LOX-1, CD11b, together with any of the signaling domains listed in the above paragraph in any combination.

### Co-Stimulatory Domains

As used herein, a “co-stimulatory molecule” or “co-stimulatory domain” refers to a molecule in an immune cell that is used to heighten or dampen an initial stimulus. For example, pathogen-associated pattern recognition receptors, such as TLR or the CD47/SIRPα axis, are molecules on immune cells that, respectively, heighten or dampen an initial stimulus. In some embodiments, a CAR co-stimulatory domain comprises TCR, CD3 zeta, CD3 gamma, CD3 delta, CD3 epsilon, CD86, common FcR gamma, FcR beta (Fc Epsilon R1b), CD79a, CD79b, Fcgamma RIIa, DAP10, DAP12, T cell receptor (TCR), CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, other co-stimulatory molecules described herein, any derivative, variant, or fragment thereof, any synthetic sequence of a co-stimulatory molecule that has the same functional capability, and any combinations thereof.

In some embodiments, a co-stimulatory domain may be a domain that is endogenous to a particular immune cell type (e.g., a modified immune cell as provided herein). In some embodiments, a co-stimulatory domain may be a domain that is not endogenous to a particular immune cell type (e.g., a modified immune cell as provided herein).

As used herein, a “co-stimulatory signal” refers to a signal, which in combination with a primary signal, such as activation of a CAR on an immune cell, leads to activation of the immune cell.

### Cleavage Peptides

As used herein, a cleavage peptide refers to a peptide that can induce the cleaving of a recombinant protein in a cell. In some embodiments, a cleavage peptide is a 2A peptide. In some embodiments, a cleavage peptide is or comprises a P2A, F2A, E2A or T2A peptide. In some embodiments, a nucleic acid as described herein comprises one or more nucleic acid sequences encoding one or more cleavage peptides. In some embodiments, a nucleic acid comprising a nucleic acid sequence encoding a cleavage peptide also comprises one or more nucleic acid sequences encoding one or more intracellular domains and one or more nucleic acid sequences comprising one or more peptide agents, wherein translation of the nucleic acid results in a protein comprising one or more intracellular domains separated from one or more peptide agents by a cleavage peptide. In some embodiments, a first promoter is operably linked to one or more nucleic acids encoding a CAR and a second promoter is operably linked to one or more nucleic acids encoding a peptide agent. In some embodiments, a nucleic acid sequence comprising a CAR, and optionally one or more peptide agents, further comprises an internal ribosome entry site (IRES) sequence. An IRES sequence may be any viral, chromosomal or artificially designed sequence that initiates cap-independent ribosome binding to mRNA facilitates the initiation of translation.

### CAR Peptide Agents

As used herein, a CAR peptide agent refers to a peptide co-expressed with a CAR in an immune cell. In some embodiments, a CAR peptide agent is co-expressed with a CAR to ensure stoichiometric balance and optimal signaling of a CAR. In some embodiments, a CAR peptide agent forms a homodimer with an identical peptide agent. In some embodiments, a CAR peptide agent forms a heterodimer with a different peptide agent. In some embodiments, a nucleic acid as described herein comprises one or more nucleic acid sequences encoding one or more CAR peptide agents. In some embodiments, a CAR peptide agent is or comprises an FcR gamma chain.

In some embodiments, a CAR peptide agent comprises any peptide, protein, receptor, secreted antibody or a fragment thereof (e.g., an scFv, Fab, Fab′, F(ab′) 2, Fc, or nanobody). In some embodiments, a CAR peptide agent comprises one or more cytokines (e.g., one or more of IL-1, IL-2, IL-6, IL-8, TNF-α, IFNa, IFNb, IFN-γ, GMCSF, or MCSF), CD40-L, dominant negative SIRPα, dominant negative PD1, dominant negative CD45, dominant negative SIGLEC 10, or dominant negative LILRB.

### Fc Receptors (FcR)

In some embodiments, a CAR comprises one or more antigen binding domains and an FcR extracellular domain, and/or the transmembrane domain of the CAR comprises an FcR transmembrane domain, and/or the intracellular domain of the CAR comprises an FcR intracellular domain. In some embodiments, a CAR comprises, from N-terminus to C-terminus, one or more extracellular domains, an FcR extracellular domain, an FcR transmembrane domain, and an FcR intracellular domain. In some embodiments, one or more of the FcR extracellular domain, the FcR transmembrane domain and the FcR intracellular domain is or comprises a human FcR domain. In some embodiments, an FcR extracellular domain, an FcR transmembrane domain and an FcR intracellular domain together comprise a full-length FcR. In some embodiments, an FcR extracellular domain, an FcR transmembrane domain and an FcR intracellular domain together comprise a portion of a full-length FcR. In some embodiments, an FcR extracellular domain comprises a portion of a full-length FcR extracellular domain. In some embodiments, an FcR transmembrane domain comprises a portion of a full-length FcR transmembrane domain. In some embodiments, an FcR intracellular domain comprises a portion of a full-length FcR intracellular domain.

### Toll-Like Antigen Receptors (TLR)

In some embodiments, a CAR comprises one or more antigen binding domains and a toll-like receptor (TLR) extracellular domain and/or the transmembrane domain of the CAR comprises a TLR transmembrane domain and/or the intracellular domain of the CAR comprises a TLR intracellular domain. In some embodiments, a CAR comprises, from N-terminus to C-terminus, one or more extracellular domains, a TLR extracellular domain, a TLR transmembrane domain, and a TLR intracellular domain. In some embodiments, one or more of the TLR extracellular domain, the TLR transmembrane domain and the TLR intracellular domain is or comprises a human TLR domain. In some embodiments, a TLR extracellular domain, a TLR transmembrane domain and a TLR intracellular domain together comprise a full-length TLR. In some embodiments, a TLR extracellular domain, a TLR transmembrane domain and a TLR intracellular domain together comprise portion of a full-length TLR. In some embodiments, a TLR extracellular domain comprises a portion of a full-length TLR extracellular domain. In some embodiments, a TLR transmembrane domain comprises a portion of a full-length TLR transmembrane domain. In some embodiments, a TLR intracellular domain comprises a portion of a full-length TLR intracellular domain.

### Membrane-Tethered Cytokines

The term “membrane-tethered cytokine”, as used herein, refers to an artificial chimeric protein comprising a cytokine fused to a membrane tether, such that the cytokine stimulates neighboring cells in trans. Membrane-tethered cytokines may be used, for example, as a therapy with adoptive cell transfer. For example, in some embodiments, immune cells (e.g., stem cells, macrophages, monocytes, and/or dendritic cells) are removed from a patient (e.g., from blood, tumor or ascites fluid) and modified so that they express a membrane-tethered cytokine. In some embodiments, such modified immune cells are then reintroduced to the same or a different patient as a therapeutic. In some embodiments, use of modified immune cells (e.g., stem cells, macrophages, monocytes, and/or dendritic cells) comprising a membrane-tethered cytokine avoids toxicity associated with systemic delivery of the cytokine.

In some embodiments, a membrane-tethered cytokine comprises an extracellular domain and a membrane tether. In some embodiments, an extracellular domain is or comprises a pro-inflammatory (M1) cytokine. In some embodiments, a pro-inflammatory cytokine is or comprises a Type I Inteferon (IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-β, IFN-ω, IFN-ε, or IFN-κ), Type II Interferon (IFN-γ), Type III Interferon (IFN-λ1, IFN-λ2, IFN-λ3, or IFN-λ4), TNF-α, IL-1β, IL-6, IL-12, IL-17, IL-23, or GM-CSF. In some embodiments, an extracellular domain is or comprises an anti-inflammatory (M2) cytokine. In some embodiments, an anti-inflammatory cytokine is or comprises IL-4, IL-10, IL-13, IL-18, M-CSF, or TGF-β. In some embodiments, a membrane-tethered cytokine comprises an extracellular domain that is or comprises a Type I Interferon (IFN-α1, IFN-α2, IFN-α4, IFN-α5, IFN-α6, IFN-α7, IFN-α8, IFN-α10, IFN-α13, IFN-α14, IFN-α16, IFN-α17, IFN-α21, IFN-β, IFN-ω, IFN-ε, or IFN-κ), Type II Interferon (IFN-γ), Type III Interferon (IFN-21, IFN-22, IFN-23, or IFN-24), TNF-α, IL-1B, IL-6, IL-12, IL-17, IL-23, GM-CSF, IL-4, IL-10, IL-13, IL-18, M-CSF, or TGF-β. In some embodiments, an extracellular domain is or comprises IFN-β.

In some embodiments, a “membrane tether” comprises a membrane-anchoring region and a spacer region. In some embodiments, a membrane-anchoring region comprises a protein-based transmembrane domain (TMD) or a lipid-based posttranslational modification (e.g., a glycolipid). In some embodiments, a protein-based TMD comprises up to 30 amino acids, preferably 18-25 amino acids. In some embodiments, a protein-based TMD adopts an alpha helical secondary structure. In some embodiments, a protein-based TMD comprises greater than 50% hydrophobic residues (e.g., isoleucine, valine, leucine, tryptophan, alanine, or methionine). In some embodiments, a protein-based TMD adopts an alpha helical secondary structure and comprises greater than 50% hydrophobic residues (e.g., isoleucine, valine, leucine, tryptophan, alanine, or methionine). In some embodiments, a protein-based TMD is derived from B7, CD86, EGFR, PDGFRA, PDGFRB, PDL1, Notch, CD4, CD8a, ICAM-1, VCAM-1, EPCAM, PECAM1, or NCAM-1. In some embodiments, a lipid-based a membrane-anchoring region comprises a glycosylphosphatidylinositol (GPI) anchor. In some embodiments, a lipid-based membrane-anchoring region comprises a C-terminal amino acid signal sequence that induces the posttranslational addition of a GPI anchor (i.e., a GPI signal sequence). In some embodiments, a GPI signal sequence is derived from Thyl, CD52, CD55, CD59, GP2, CD87, or folate receptor 1. In some embodiments, a membrane-anchoring region is fused to a spacer region. In some embodiments, a spacer region comprises up to 120 amino acids, preferably 10-48 amino acids. In some embodiments, a spacer sequence comprises a glycine-serine linker; a peptide that can be cleaved by a MMP; an immunoglobulin (Ig)-like domain from CD28, CD33, ILIR1, IL1R2, SIGLEC1, SIGLEC7, or Thyl; or at least 20 amino acids from said Ig-like domains. In some embodiments, a membrane tether is or comprises: a B7 transmembrane domain (TMD) membrane-anchoring region; a B7 TMD membrane-anchoring region with a matrix metalloproteinase (MMP) linker; a glycosylphosphatidylinositol (GPI) membrane-anchoring region; or a GPI membrane-anchoring region with a CD28 spacer region. In some embodiments, a membrane tether can release from the modified immune cell upon binding of the extracellular domain with a receptor expressed by another cell.

In some embodiments, a membrane-tethered cytokine of the present disclosure comprises an amino acid sequence at least 80% identical to an amino acid sequence selected from Table 6. In some embodiments, a membrane-tethered cytokine of the present disclosure comprises an amino acid sequence at least 85% identical to an amino acid sequence selected from Table 6. In some embodiments, a membrane-tethered cytokine of the present disclosure comprises an amino acid sequence at least 90% identical to an amino acid sequence selected from Table 6. In some embodiments, a membrane-tethered cytokine of the present disclosure comprises an amino acid sequence at least 95% identical to an amino acid sequence selected from Table 6. In some embodiments, a membrane-tethered cytokine of the present disclosure comprises an amino acid sequence at least 96% identical to an amino acid sequence selected from Table 6. In some embodiments, a membrane-tethered cytokine of the present disclosure comprises an amino acid sequence at least 97% identical to an amino acid sequence selected from Table 6. In some embodiments, a membrane-tethered cytokine of the present disclosure comprises an amino acid sequence at least 98% identical to an amino acid sequence selected from Table 6. In some embodiments, a membrane-tethered cytokine of the present disclosure comprises an amino acid sequence at least 99% identical to an amino acid sequence selected from Table 6. In some embodiments, a membrane-tethered cytokine of the present disclosure comprises an amino acid sequence identical to an amino acid sequence selected from Table 6.

In some embodiments, a membrane-tethered cytokine of the present disclosure is encoded by one or more nucleic acids comprising a sequence at least 80% identical to a nucleotide sequence selected from Table 6. In some embodiments, a membrane-tethered cytokine of the present disclosure is encoded by one or more nucleic acids comprising a sequence at least 85% identical to a nucleotide sequence selected from Table 6. In some embodiments, a membrane-tethered cytokine of the present disclosure is encoded by one or more nucleic acids comprising a sequence at least 90% identical to a nucleotide sequence selected from Table 6. In some embodiments, a membrane-tethered cytokine of the present disclosure is encoded by one or more nucleic acids comprising a sequence at least 95% identical to a nucleotide sequence selected from Table 6. In some embodiments, a membrane-tethered cytokine of the present disclosure is encoded by one or more nucleic acids comprising a sequence at least 96% identical to a nucleotide sequence selected from Table 6. In some embodiments, a membrane-tethered cytokine of the present disclosure is encoded by one or more nucleic acids comprising a sequence at least 97% identical to a nucleotide sequence selected from Table 6. In some embodiments, a membrane-tethered cytokine of the present disclosure is encoded by one or more nucleic acids comprising a sequence at least 98% identical to a nucleotide sequence selected from Table 6. In some embodiments, a membrane-tethered cytokine of the present disclosure is encoded by one or more nucleic acids comprising a sequence at least 99% identical to a nucleotide sequence selected from Table 6. In some embodiments, a membrane-tethered cytokine of the present disclosure is encoded by one or more nucleic acids comprising a sequence identical to a nucleotide sequence selected from Table 6.

### Nucleic Acid Constructs

In some embodiments, the present disclosure provides nucleic acid molecules encoding at least one CAR described herein or a fragment thereof. The present disclosure also provides, among other things, nucleic acid molecules encoding at least one membrane-tethered cytokine described herein or a fragment thereof. An immune cell (e.g., stem cell, macrophage, monocyte, or dendritic cell) can comprise a nucleic acid molecule (e.g., an exogenous nucleic acid molecule) encoding at least one protein (e.g., a CAR of the present disclosure or a membrane-tethered cytokine of the present disclosure) described herein.

Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns to the extent that the nucleotide sequence encoding the protein may in some version contain an intron(s). The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “operably linked” or “transcriptional control” refers to functional linkage between a regulatory sequence and a heterologous nucleic acid sequence resulting in expression of the heterologous nucleic acid sequence. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

Nucleic acid molecules encoding at least one protein (e.g., a CAR of the present disclosure or a membrane-tethered cytokine of the present disclosure) described herein or a fragment thereof can be a DNA molecule, an RNA molecule, or a combination thereof. In some embodiments, a nucleic acid molecule comprises or is a messenger RNA (mRNA) transcript encoding at least one protein (e.g., a CAR of the present disclosure or a membrane-tethered cytokine of the present disclosure) described herein or a fragment thereof. In some embodiments, a nucleic acid molecule comprises or is a DNA construct encoding at least one protein (e.g., a CAR of the present disclosure or a membrane-tethered cytokine of the present disclosure) described herein or a fragment thereof.

In some embodiments, all or a fragment of a protein (e.g., a CAR of the present disclosure or a membrane-tethered cytokine of the present disclosure) described herein is encoded by a codon optimized nucleic acid molecule, e.g., for expression in a cell (e.g., a mammalian cell). A variety of codon optimization methods are known in the art, e.g., as disclosed in U.S. Pat. Nos. 5,786,464 and 6,114,148, each of which is hereby incorporated by reference in its entirety.

Expression of nucleic acids as described herein may be achieved by operably linking a nucleic acid encoding a protein (e.g., a CAR of the present disclosure or a membrane-tethered cytokine of the present disclosure) or fragment thereof to a promoter in an expression vector. Exemplary promoters (e.g., constitutive promoters) include, but are not limited to, an elongation factor-1α promoter (EF-1α) promoter, immediate early cytomegalovirus (CMV) promoter, ubiquitin C promoter, phosphoglycerokinase (PGK) promoter, simian virus 40 (SV40) early promoter, mouse mammary tumor virus (MMTV) promoter, human immunodeficiency virus (HIV) long terminal repeat (LTR) promoter, Moloney murine leukemia virus (MoMuLV) promoter, an avian leukemia virus promoter, an Epstein-Barr virus immediate early promoter, a Rous sarcoma virus promoter, an actin promoter, a myosin promoter, a hemoglobin promoter, or a creatine kinase promoter. Examples of inducible promoters include, but are not limited to a metallothionine promoter, a glucocorticoid promoter, a progesterone promoter, and a tetracycline promoter. A vector can also comprise additional promoter elements, e.g., enhancers, to regulate the frequency of transcriptional initiation.

In some embodiments, a vector comprising a nucleic acid molecule encoding a protein (e.g., a CAR of the present disclosure or a membrane-tethered cytokine of the present disclosure) or fragment thereof comprises or is a viral vector. Viral vector technology is well known in the art and is described (e.g., in Sambrook et al., 2012, MOLECULAR CLONING: A LABORATORY MANUAL, volumes 1-4, Cold Spring Harbor Press, NY). Examples of viral vectors include, but are not limited to, adenoviral vectors, adeno-associated viral vectors, or retroviral vectors (e.g., a lentiviral vector or a gammaretroviral vector). In some embodiments, a vector comprises a lentiviral vector (e.g., as described in U.S. Pat. No. 9,149,519 or International Publication No. WO 2017/044487, each of which is hereby incorporated by reference in its entirety).

In some embodiments, a viral vector comprises an adenoviral vector. Adenoviruses are a large family of viruses containing double stranded DNA. They replicate within the nucleus of a host cell, using the host's cell machinery to synthesize viral RNA, DNA and proteins. Adenoviruses are known in the art to affect both replicating and non-replicating cells, to accommodate large transgenes, and to code for proteins without integrating into the host cell genome. In some embodiments, an adenoviral vector comprises an Ad2 vector or an Ad5 vector (e.g., Ad5f35 adenoviral vector, e.g., a helper-dependent Ad5F35 adenoviral vector).

In some embodiments, a viral vector is an adeno-associated virus (AAV) vector. AAV systems are generally well known in the art (see, e.g., Kelleher and Vos, Biotechniques, 17 (6): 1110-17 (1994); Cotten et al., P.N.A.S. U.S.A., 89 (13): 6094-98 (1992); Curiel, Nat Immun, 13 (2-3): 141-64 (1994); Muzyczka, Curr Top Microbiol Immunol, 158:97-129 (1992); and Asokan A, et al., Mol. Ther., 20 (4): 699-708 (2012)). Methods for generating and using recombinant AAV (rAAV) vectors are described, for example, in U.S. Pat. Nos. 5,139,941 and 4,797,368.

Several AAV serotypes have been characterized, including AAV1, AAV2, AAV3 (e.g., AAV3B), AAV4, AAV5, AAV6, AAV7, AAV8, AAV9, AAV10, and AAV11, as well as variants thereof. Generally, any AAV serotype may be used to deliver a protein (e.g., a CAR of the present disclosure or a membrane-tethered cytokine of the present disclosure) or fragment thereof described herein. In some embodiments, an AAV serotype has a tropism for a particular tissue.

In some embodiments, CRISPR/Cas9 system has recently been shown to facilitate high levels of precise genome editing using adeno associated viral (AAV) vectors to serve as donor template DNA during homologous recombination (HR).

In some embodiments, a vector comprises a gammaretroviral vector (e.g., as described in Tobias Maetzig et al., “Gammaretroviral Vectors: Biology, Technology and Application” Viruses. 2011 June; 3 (6): 677-713, which is hereby incorporated by reference in its entirety). Exemplary gammaretroviral vectors include Murine Leukemia Virus (MLV), Spleen-Focus Forming Virus (SFFV), and Myeloproliferative Sarcoma Virus (MPSV), and vectors derived therefrom.

In some embodiments, a vector comprises two or more nucleic acid sequences encoding proteins, e.g., at least one CAR described herein and/or membrane-tethered cytokine described herein. In some embodiments, a vector comprises two or more nucleic acid sequences encoding proteins, e.g., at least one CAR described herein and/or membrane-tethered cytokine described herein. In some embodiments, two or more nucleic acid sequences encoding a CAR and/or a membrane-tethered cytokine are encoded by a single nucleic molecule, e.g., in same frame and as a single polypeptide chain. In some embodiments, two or more nucleic acid sequences encoding a CAR and/or a membrane-tethered cytokine are encoded by a single nucleic molecule, e.g., in same frame and as a single polypeptide chain. In some embodiments, two or more proteins (e.g., CAR and/or a membrane-tethered cytokine) are separated by one or more cleavage peptide sites (e.g., an auto-cleavage site or a substrate for an intracellular protease). In some embodiments, two or more proteins (e.g., CAR and/or a membrane-tethered cytokine) are separated by one or more cleavage peptide sites (e.g., an auto-cleavage site or a substrate for an intracellular protease). In certain embodiments, a cleavage peptide comprises a porcine teschovirus-1 (P2A) peptide, Thosea asigna virus (T2A) peptide, equine rhinitis A virus (E2A) peptide, foot-and-mouth disease virus (F2A) peptide, or a variant thereof.

In some embodiments, a vector comprises at least one nucleic acid sequence encoding a protein, e.g., at least one CAR described herein or at least one membrane-tethered cytokine described herein, and at least one nucleic acid encoding at least one gene co-expressed with a second protein, e.g., a cytokine described herein (e.g., TNF, IL-12, IFN, GM-CSF, G-CSF, M-CSF, and/or IL-1) or a stimulatory ligand described herein (e.g., CD7, B7-1 (CD80), B7-2 (CD86), PD-L1, PD-L2, 4-1BBL, OX40L, ICOS-L, ICAM, CD30L, CD40, CD40L, CD70, CD83, HLA-G, MICA, MICB, HVEM, lymphotoxin beta receptor, 3/TR6, ILT3, ILT4, HVEM, an agonist or antibody that binds Toll ligand receptor, and/or a B7-H3 ligand).

### Pharmaceutical Compositions

The present disclosure, among other things, provides pharmaceutical compositions comprising modified immune cells (e.g., stem cells, macrophages, monocytes, or dendritic cells) comprising a CAR as described herein and/or a membrane-tethered cytokine as described herein with one or more pharmaceutically or physiologically acceptable carriers, diluents, or excipients. The present disclosure, among other things, also provides pharmaceutical compositions comprising nucleic acids encoding a CAR as described herein and/or a membrane-tethered cytokine as described herein in combination with one or more pharmaceutically or physiologically acceptable carriers, diluents, or excipients.

When “a therapeutically effective amount”, “an immunologically effective amount,” “an anti-immune response effective amount,” or “an immune response-inhibiting effective amount” is indicated, a precise amount of a pharmaceutical composition described herein can be determined by a physician with consideration of individual differences in age, weight, immune response, and condition of the patient (subject).

Pharmaceutical compositions described herein may comprise buffers, such as neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); serum and preservatives, such as cryoprotectant. In some embodiments, a pharmaceutical composition is substantially free of contaminants, e.g., there are no detectable levels of a contaminant (e.g., an endotoxin).

Pharmaceutical compositions described herein may be administered in a manner appropriate to the disease, disorder, or condition to be treated or prevented. Quantity and frequency of administration will be determined by such factors as condition of a patient, and type and severity of a patient's disease, disorder, or condition, although appropriate dosages may be determined by clinical trials.

Pharmaceutical compositions described herein may be in a variety of forms. These include, for example, liquid, semi-solid and solid dosage forms, such as liquid solutions (e.g., injectable and infusible solutions), dispersions or suspensions, liposomes, and suppositories. Preferred compositions may be injectable or infusible solutions. Pharmaceutical compositions described herein can be formulated for administration intravenously, subcutaneously, intradermally, intratumorally, intranodally, intramedullary, intramuscularly, transarterially, or intraperitoneally.

In some embodiments, a pharmaceutical composition described herein is formulated for parenteral (e.g., intravenous, subcutaneous, intraperitoneal, or intramuscular) administration. In some embodiments, a pharmaceutical composition described herein is formulated for intravenous infusion or injection. In some embodiments, a pharmaceutical composition described herein is formulated for intramuscular or subcutaneous injection. Pharmaceutical compositions described herein can be formulated for administered by using infusion techniques that are commonly known in immunotherapy (See, e.g., Rosenberg et al., New Eng. J. of Med. 319:1676, 1988, which is hereby incorporated by reference in its entirety).

As used herein, the terms “parenteral administration” and “administered parenterally” refer to modes of administration other than enteral and topical administration, usually by injection or infusion, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoral, and intrasternal injection and infusion.

Pharmaceutical compositions comprising modified immune cells as described herein may be administered at a dosage of about 104 to about 10⁹ cells/kg body weight (e.g., about 105 to about 10⁶ cells/kg body weight), including all integer values within those ranges. In some embodiments, a dose of immune cells as described herein (e.g., stem cells, macrophages, monocytes, or dendritic cells) comprises at least about 1×10⁶, about 1.1×10⁶, about 2×10⁶, about 3.6×10⁶, about 5×10⁶, about 1×10⁷, about 1.8×10⁷, about 2×10⁷, about 5×10⁷, about 1×10⁸, about 2×10⁸, about 5×10⁸, about 1×10⁹, about 2×10⁹, or about 5×10⁹ cells. Pharmaceutical compositions described herein may also be administered multiple times at a certain dosage. An optimal dosage and treatment regime for a particular patient can readily be determined by one skilled in the art by monitoring a patient for signs of a disease, disorder, or condition and adjusting treatment accordingly.

It may be desired to administer pharmaceutical compositions described herein to a subject and then subsequently redraw blood (or have apheresis performed), activate collected immune cells, and reinfuse a subject with activated immune cells. This process can be performed multiple times, e.g., every few weeks. Immune cells (e.g., stem cells, macrophages, monocytes, or dendritic cells) can be activated from blood draws of from about 10 cc to about 400 cc. In some embodiments, immune cells (e.g., macrophages, monocytes, or dendritic cells) are activated from blood draws of about 20 cc, about 30 cc, about 40 cc, about 50 cc, about 60 cc, about 70 cc, about 80 cc, about 90 cc, or about 100 cc. Without being bound by theory, methods comprising multiple blood draw and reinfusions as described herein may select for certain immune cell populations.

In some embodiments, pharmaceutical compositions described herein are administered in combination with (e.g., before, simultaneously, or following) a second therapy. For example, a second therapy can include, but is not limited to antiviral therapy (e.g., cidofovir, interleukin-2, Cytarabine (ARA-C), or natalizumab), chimeric antigen receptor-T cell (CAR-T) therapy, T-cell receptor (TCR)-T cell therapy, chemotherapy, radiation, an immunosuppressive agent (e.g., cyclosporin, azathioprine, methotrexate, mycophenolate, FK506 antibody, or glucocorticoids), an antagonist (e.g., one or more of a PD-1 antagonist, a PD-L1 antagonist, CTLA4 antagonist, CD47 antagonist, SIRPα antagonist, CD40 agonists, CSF1/CSFIR antagonist, or a STING agonist), or an immunoablative agent (e.g., an anti-CD52 antibody (e.g., alemtuzumab), an anti-CD3 antibody, cytoxin, fludaribine, cyclosporin, FK506, rapamycin, mycophenolic acid, a steroid, FR901228, or irradiation.

In some embodiments, pharmaceutical compositions described herein are administered in combination with (e.g., before, simultaneously, or following) bone marrow transplantation or lymphocyte ablative therapy using a chemotherapy agent (e.g., fludarabine, external-beam radiation therapy (XRT), cyclophosphamide, or Rituxan). In certain embodiments, subjects undergo standard treatment with high dose chemotherapy followed by peripheral blood stem cell transplantation. In certain embodiments, following transplant, subjects receive an infusion of a pharmaceutical composition comprising immune cells as described herein. Pharmaceutical compositions described herein may be administered before or following surgery.

A dosage of any aforementioned therapy to be administered to a subject will vary with a disease, disorder, or condition being treated and based on a specific subject. Scaling of dosages for human administration can be performed according to art-accepted practices. For example, a dose of alemtuzumab will generally be about 1 mg to about 100 mg for an adult, usually administered daily for a period of between about 1 day to about 30 days, e.g., a daily dose of about 1 mg to about 10 mg per day (e.g., as described in U.S. Pat. No. 6,120,766, which is hereby incorporated by reference in its entirety).

### Methods of Treatment

The present disclosure, among other things, provides methods of treating a disease or disorder (e.g., a disease or a disorder described herein) in a subject comprising delivering a pharmaceutical composition described herein. In some embodiments, a therapeutically effective amount of a pharmaceutical composition described herein is administered to a subject having a disease or disorder. Pharmaceutical compositions as described herein can be for use in the manufacture of a medicament for treating a disease or disorder in a subject or stimulating an immune response in a subject.

The present disclosure also provides, among other things, methods of treating a disease or disorder in a subject in need thereof, the method comprising administering to the subject a therapeutically effective amount of a composition as described herein comprising: (a) one or more nucleic acid molecules, wherein the one or more intracellular moieties comprise a first JAK binding domain (or a portion thereof) and a first STAT binding domain (or a portion thereof), at least one membrane-tethered cytokine, or combinations thereof as described herein, and (b) a delivery vehicle. Accordingly, in some embodiments, following administration of the composition, one or more nucleic acid molecules are translated in an immune cell (e.g., stem cell, macrophage, monocyte, or dendritic cell) to produce a modified immune cell comprising the CAR and/or membrane-tethered cytokine. In some embodiments, at least one sign or symptom of the disease or disorder is improved in the subject after administration of a composition as described herein.

A subject to be treated with methods described herein can be a mammal, e.g., a primate, e.g., a human (e.g., a patient having, or at risk of having, a disease or disorder described herein). In some embodiments, modified immune cells (e.g., stem cells, macrophages, monocytes, or dendritic cells) may be autologous, allogeneic, or xenogeneic with respect to a subject. Pharmaceutical compositions as described herein can be administered to a subject in accordance with a dosage regimen described herein, alone or in combination with one or more therapeutic agents, procedures, or modalities.

Pharmaceutical compositions described herein can be used to treat or prevent a disease associated with a tumor or cancer, a neurodegenerative disease or disorder, an inflammatory disease or disorder, a cardiovascular disease or disorder, a fibrotic disease or disorder, a disease associated with amyloidosis, and a combination of thereof.

A method of treating (e.g., one or more of reducing, inhibiting, or delaying progression of) a cancer or a tumor in a subject with a pharmaceutical composition described herein is provided. A subject can have an adult or pediatric form of cancer. A cancer may be at an early, intermediate, or late stage, or a metastatic cancer. A cancer can include, but is not limited to, a solid tumor, a hematological cancer (e.g., leukemia, lymphoma, or myeloma, e.g., multiple myeloma), or a metastatic lesion. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma, e.g., a cutaneous melanoma), pancreas, and bones (e.g., a chordoma).

In some embodiments, a cancer is selected from a lung cancer (e.g., a non-small cell lung cancer (NSCLC) (e.g., a non-small cell lung cancer (NSCLC) with squamous and/or non-squamous histology, or a NSCLC adenocarcinoma), or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS), a prostate cancer, a breast cancer (e.g., a breast cancer that does not express one, two or all of estrogen receptor, progesterone receptor, or Her2/neu, e.g., a triple negative breast cancer), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), a nasopharyngeal cancer, a duodenal cancer, an endometrial cancer, a pancreatic cancer, a head and neck cancer (e.g., head and neck squamous cell carcinoma (HNSCC)), an anal cancer, a gastro-esophageal cancer, a thyroid cancer (e.g., anaplastic thyroid carcinoma), a cervical cancer (e.g., a squamous cell carcinoma of the cervix), a neuroendocrine tumor (NET) (e.g., an atypical pulmonary carcinoid tumor), a lymphoproliferative disease (e.g., a post-transplant lymphoproliferative disease), a lymphoma (e.g., T-cell lymphoma, B-cell lymphoma, or a non-Hodgkin lymphoma), a myeloma (e.g., a multiple myeloma), or a leukemia (e.g., a myeloid leukemia or a lymphoid leukemia).

In some embodiments, a cancer is a brain tumor, e.g., a glioblastoma, a gliosarcoma, or a recurrent brain tumor. In some embodiments, a cancer is a pancreatic cancer, e.g., an advanced pancreatic cancer. In some embodiments, a cancer is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, a cancer is a renal cancer, e.g., a renal cell carcinoma (RCC) (e.g., a metastatic renal cell carcinoma). In some embodiments, a cancer is a breast cancer, e.g., a metastatic breast carcinoma or a stage IV breast carcinoma, e.g., a triple negative breast cancer (TNBC). In some embodiments, a cancer is a virus-associated cancer. In some embodiments, a cancer is an anal canal cancer (e.g., a squamous cell carcinoma of the anal canal). In some embodiments, a cancer is a cervical cancer (e.g., a squamous cell carcinoma of the cervix). In some embodiments, a cancer is a gastric cancer (e.g., an Epstein Barr Virus (EBV) positive gastric cancer, or a gastric or gastro-esophageal junction carcinoma). In some embodiments, a cancer is a head and neck cancer (e.g., an HPV positive and negative squamous cell cancer of the head and neck (SCCHN)). In some embodiments, a cancer is a nasopharyngeal cancer (NPC). In some embodiments, a cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer.

In some embodiments, a cancer is a hematological cancer. In some embodiments, a cancer is a leukemia, e.g., acute myeloid leukemia, chronic myeloid leukemia, acute lymphoblastic leukemia, chronic lymphocytic leukemia, chronic leukemia, or acute leukemia. In some embodiments, a cancer is a lymphoma, e.g., Hodgkin lymphoma (HL), non-Hodgkin's lymphoma, lymphocytic lymphoma, or diffuse large B cell lymphoma (DLBCL) (e.g., a relapsed or refractory HL or DLBCL). In some embodiments, a cancer is a myeloma, e.g., multiple myeloma.

Pharmaceutical compositions described herein can be used to enhance or modulate an immune response in a subject. In one embodiment, a pharmaceutical composition described herein enhances, stimulates, or increases an immune response in a subject (e.g., a subject having, or at risk of, a disease or disorder described herein). In certain embodiments, a subject is, or is at risk of being, immunocompromised. For example, a subject is undergoing or has undergone a chemotherapeutic treatment and/or radiation therapy.

In some embodiments, a subject has, or is at risk of, developing an inflammatory disorder (e.g., a chronic or acute inflammatory disorder). In some embodiments, a subject has, or is at risk, of developing an autoimmune disease or disorder. Exemplary autoimmune diseases that can be treated with methods described herein include, but are not limited to, Alzheimer's disease, asthma (e.g., bronchial asthma), an allergy (e.g., an atopic allergy), Acquired Immunodeficiency Syndrome (AIDS), atherosclerosis, Behcet's disease, celiac, cardiomyopathy, Crohn's disease, cirrhosis, diabetes, diabetic retinopathy, eczema, fibromyalgia, fibromyositis, glomerulonephritis, graft vs. host disease (GVHD), Guillain-Barre syndrome, hemolytic anemia, multiple sclerosis, myasthenia gravis, osteoarthritis, polychondritis, psoriasis, rheumatoid arthritis, sepsis, stroke, vasculitis, ventilator-induced lung injury, transplant rejection, Raynaud's phenomena, Reiter's syndrome, rheumatic fever, sarcoidosis, scleroderma, Sjogren's syndrome, ulcerative colitis, uveitis, vitiligo, or Wegener's granulomatosis.

Administration of pharmaceutical compositions described herein may be carried out in any convenient manner (e.g., injection, ingestion, transfusion, inhalation, implantation, or transplantation). In some embodiments, a pharmaceutical composition described herein is administered by injection or infusion. Pharmaceutical compositions described herein may be administered to a patient transarterially, subcutaneously, intravenously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by intravenous infusion or injection. In some embodiments, a pharmaceutical composition described herein is administered by intramuscular or subcutaneous injection. Pharmaceutical compositions described herein may be injected directly into a site of inflammation, a local disease site, a lymph node, an organ, a tumor, or site of infection in a subject.

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. An example is provided for illustrative purposes only. It is not to be construed as limiting the scope or content of the disclosure in any way.

### EXAMPLES

The following examples are provided so as to describe to the skilled artisan how to make and use methods and compositions described herein, and are not intended to limit the scope of the present disclosure.

### Example 1: Generation of JAK/STAT CAR Constructs and Effects on STAT Signaling in Macrophages

The present Example describes generation of JAK/STAT CAR constructs and assesses cytokine secretion and STAT1 phosphorylation in macrophages expressing such constructs.

JAK/STAT CAR constructs were generated by engineering specific domains from G-CSF and IFNγ cytokine receptors into CAR constructs. A JAK binding domain derived from the G-CSF cytokine receptor was used. G-CSF receptor typically drives STAT3 and STAT5 signaling, however STAT1 signaling is typically the main driver of IFNγ response. Therefore, a minimal STAT1 binding domain derived from the IFNγ cytokine receptor was used.

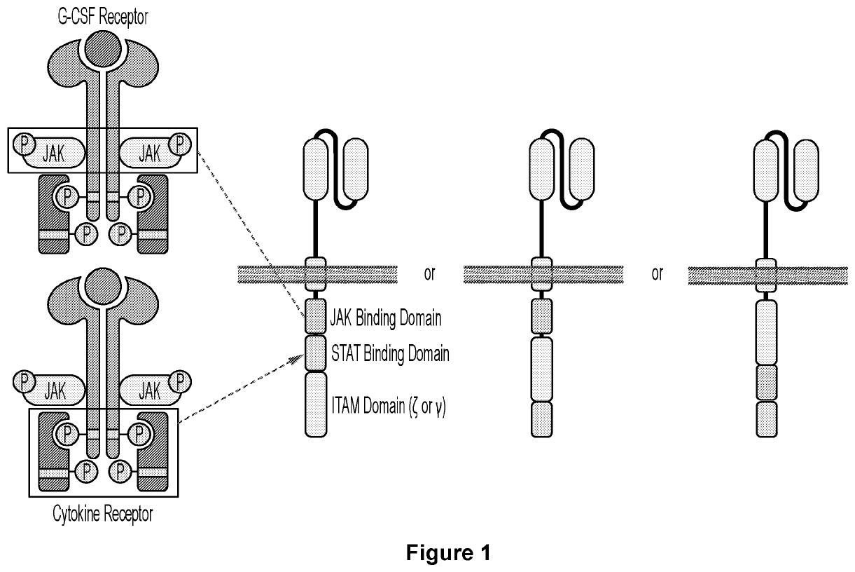

Orientation of these domains (e.g., JAK binding domain, STAT binding domain, and ITAM domain) within a CAR construct may directly influence CAR function. Without wishing to be bound by any particular theory, it is predicted that when JAK/STAT binding domains are membrane proximal (e.g., relative to ITAM domain), STAT signaling is constitutively active but ITAM signaling is not. Thus, in the absence of target antigen, these CAR constructs may still lead to polarization of macrophages toward an M1 state and secretion of specific IFNγ cytokines. Without wishing to be bound by any particular theory, it is predicted that when JAK/STAT binding domains are membrane distal (e.g., relative to ITAM domain), STAT signaling is inducible upon antigen binding. Thus, in order to elicit M1 polarization and IFNγ-induced cytokine secretion, these CAR constructs must engage with their target antigen. The ability to achieve either an inducible or constitutive CAR system allows for potentially broad applications of such CAR constructs. Exemplary JAK/STAT CAR constructs (e.g., membrane proximal and membrane distal) are shown in FIG. 1.

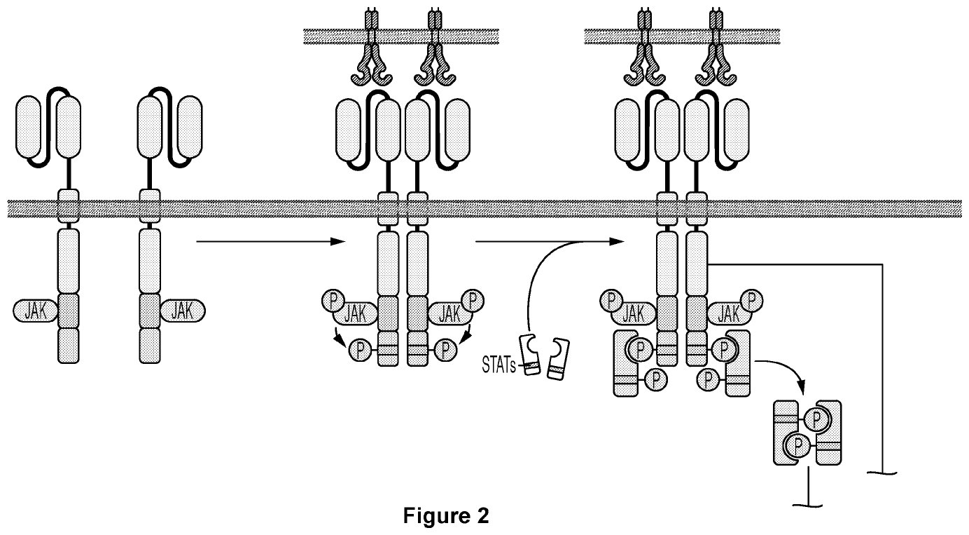

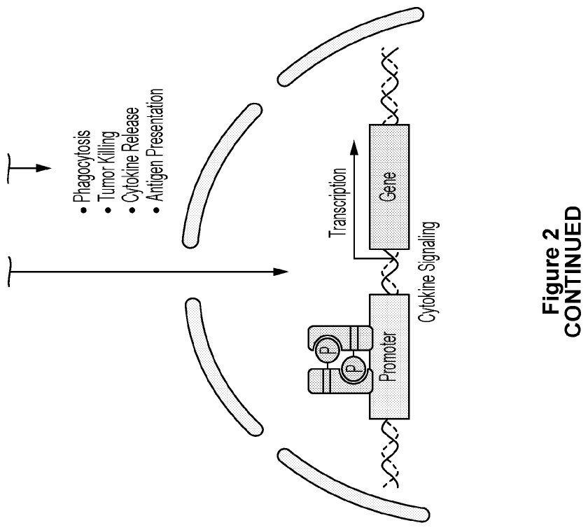

JAK/STAT proteins are integral components of cytokine signaling. Incorporation of JAK/STAT domains into CAR constructs may result in various effects in cells (e.g., macrophages) in which the CAR constructs are expressed. JAK/STAT CAR constructs (e.g., constitutive and/or inducible constructs) may result in cytokine secretion, differentiation, polarization, and changes in phenotype as shown in FIG. 2.

Macrophages were thawed and plated at a density of 2e6 cells per well in 6-well UpCell plates in 2 mL TexMacs+10% FBS+1% Pen/Strep+10 ng/ml GM-CSF (complete media; CM). After 2-3 hours of rest, VPX lentivirus particles were diluted into CM and added to macrophages at specified MOIs. Media was fully exchanged 24 hours post-lentivirus addition. Cells were tested 6-7 days post transduction.

### Cytokine Secretion

HER2 and Mesothelin antigens were diluted in PBS to a concentration of 28 nM. 100 μL of antigen solution or control PBS was added to wells and incubated at 4° C. for 24 hours so that the antigens passively bound to the plate. Wells were then washed twice with PBS and 50,000 macrophages were added to each well. Plates were incubated for 24 hours, followed by centrifugation at 300×g for 5 minutes. Supernatant was removed and stored at −20° C. Cytokine levels were assessed using Proinflammatory Human Kit and Chemokine Human Kit from MSD following standard protocol instructions.

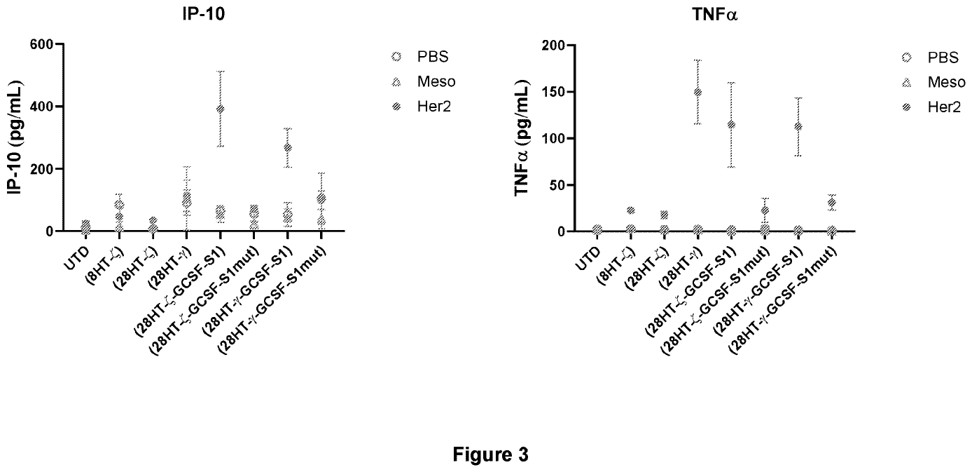

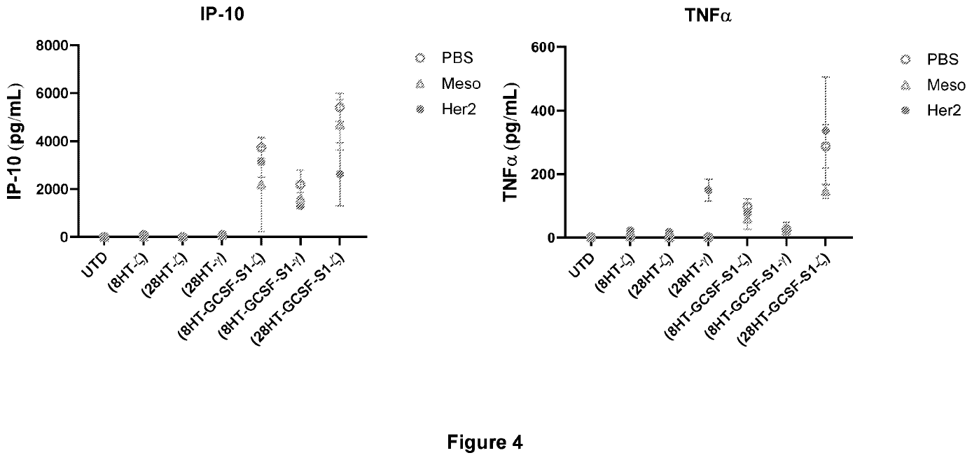

Supernatant cytokine levels of IP-10 and TNFα from macrophages expressing CAR constructs comprising JAK/STAT domains distal to plasma membrane are shown in FIG. 3. Cytokine secretion was observed only in macrophages incubated with HER2 antigen. Thus, as predicted, STAT1 signaling was inducible in CAR constructs comprising JAK/STAT domains distal to plasma membrane. Supernatant cytokine levels of IP-10 and TNFα from macrophages expressing CAR constructs comprising JAK/STAT domains proximal to plasma membrane are shown in FIG. 4. Cytokine secretion was observed in macrophages incubated with PBS control, Mesothelin antigen, and HER2 antigen. Thus, as predicted, STAT1 signaling was constitutive in CAR constructs comprising JAK/STAT domains proximal to the plasma membrane.

### STAT1 Phosphorylation

Macrophages were transduced with lentivirus encoding CAR constructs targeted to HER2 comprising various intracellular elements in particular orders. 6 days after transduction, cells were lifted and re-plated onto UpCell tissue culture plates coated with 5 μg/mL HER2 or PBS control. Samples were then harvested by scraping cells at 1 hour and 2 hours post-stimulation time points and were then snap-frozen on dry ice. Positive control samples were stimulated using either 100 U/mL IFNγ or 100 ng/mL IFNβ for 30 minutes and harvested. Frozen pellets were lysed using RIPA buffer with phosphatase inhibitors and total cell lysates were assayed via Western Blot using antibodies probing for phosphorylated STAT1 and GAPDH protein as loading control.

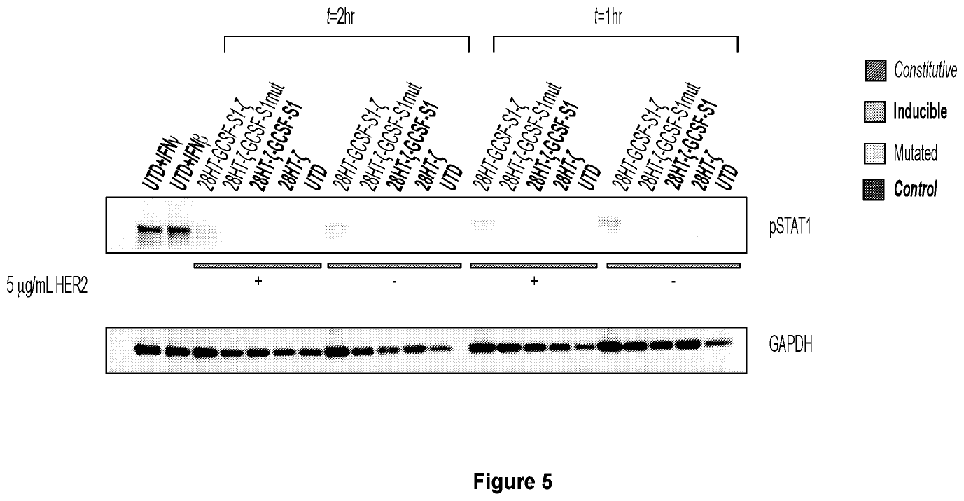

Phosphorylated STAT1 levels are shown in FIG. 5. STAT1 was phosphorylated in macrophages expressing a constitutive CAR construct when incubated with HER2 antigen or PBS control. As predicted, and consistent with cytokine expression studies described above, CAR constructs comprising JAK/STAT domains proximal to the plasma membrane resulted in constitutively active STAT1 signaling.

### Example 2: Functions of Constitutive, Inducible, and Mutated JAK/STAT CAR Constructs in Macrophages

The present Example assesses cell viability, CAR expression, M1/M2 phenotypic marker expression, and tumor killing function in macrophages expressing constitutive, inducible, and mutated JAK/STAT CAR constructs.

Macrophages were thawed and plated at a density of 2e6 cells per well in 6-well UpCell plates in 2 mL TexMacs+10% FBS+1% Pen/Strep+10 ng/ml GM-CSF (complete media; CM). After 2-3 hours of rest, VPX lentivirus particles were diluted into CM and added to macrophages at specified MOIs. Media was fully exchanged 24 hours post-lentivirus addition. Cells were tested 6-7 days post transduction.

VPX lentivirus particles utilized in these experiments encoded CAR constructs targeted to HER2 comprising various intracellular elements in particular orders. JAK/STAT CAR constructs utilized in these experiments can be categorized as constitutive, inducible, or mutated. Mutated CAR constructs contained a STAT1 binding domain in which the tyrosine residue that is phosphorylated by JAK proteins was changed to a phenylalanine, thus inhibiting phosphorylation and causing loss of STAT1 binding function.

For assessment of cell viability, CAR expression, and M1/M2 markers, macrophages were plated at 50,000 cells per well and stained with Aqua Live/Dead, APC-anti-Trastuzumab, FITC-CD80, PE-CD86, PerCP-Cy5.5-CD163, and BV421-CD206. Flow cytometry was performed using Attune Cytometer (ThermoFischer).

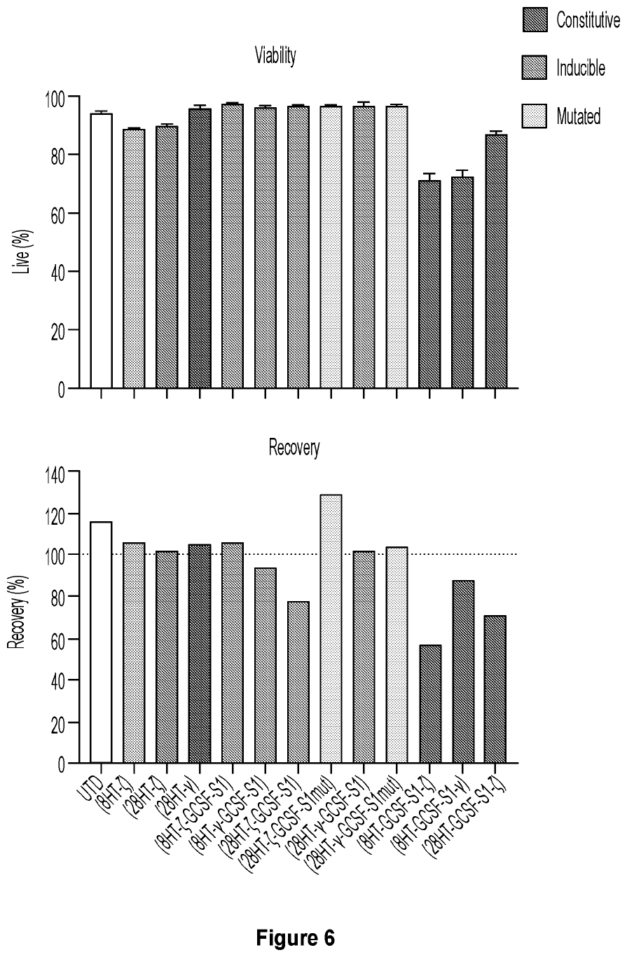

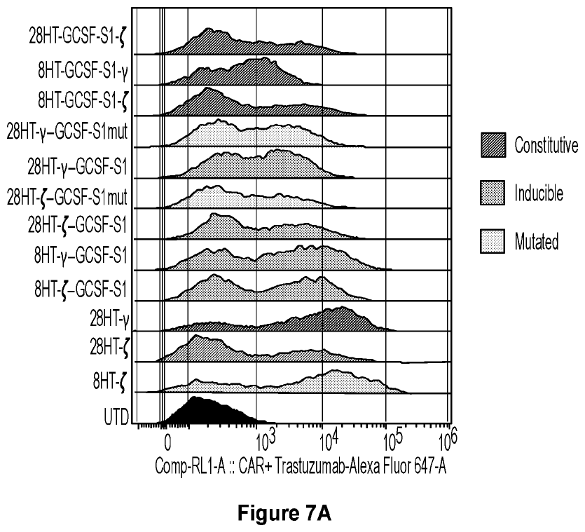

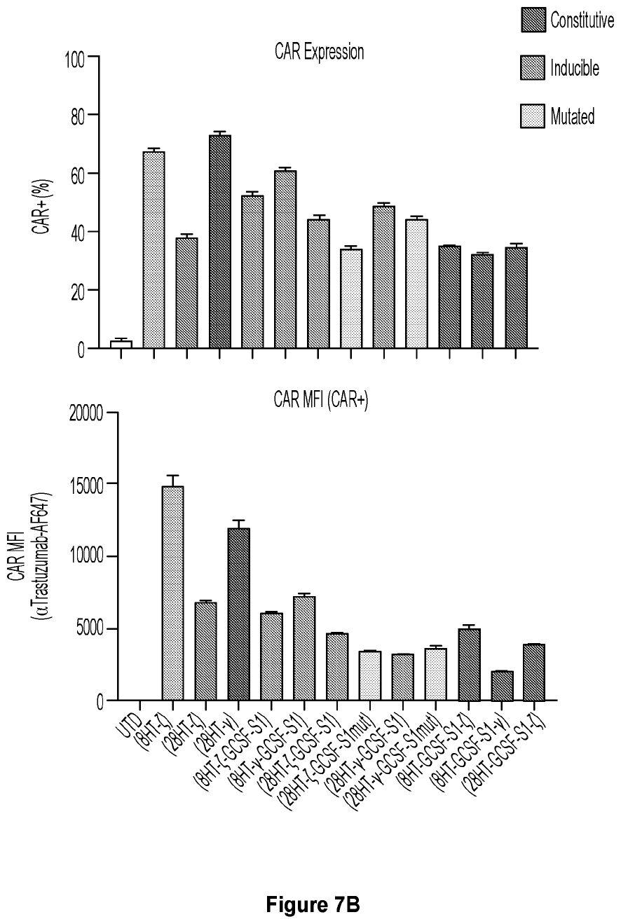

Cell viability and recovery are shown in FIG. 6. Recovery is defined (in this context) as the number of live cells post-lifting on day 6 or 7 divided by total number of cells initially seeded for transduction with lentivirus at day 0. Viability is defined (in this context) as the percentage of events captured via flow cytometry that are negative for viability dye. Expression of JAK/STAT CAR constructs were found to have minimal effects on cell viability and recovery. Constitutive, inducible, and mutated CAR construct expression is shown via histograms in FIG. 7A. Quantification of CAR expression, both in terms of percentage of cells expressing CAR construct and mean fluorescence intensity, is shown in FIG. 7B. JAK/STAT CAR constructs were found to express at the cell surface of macrophages.

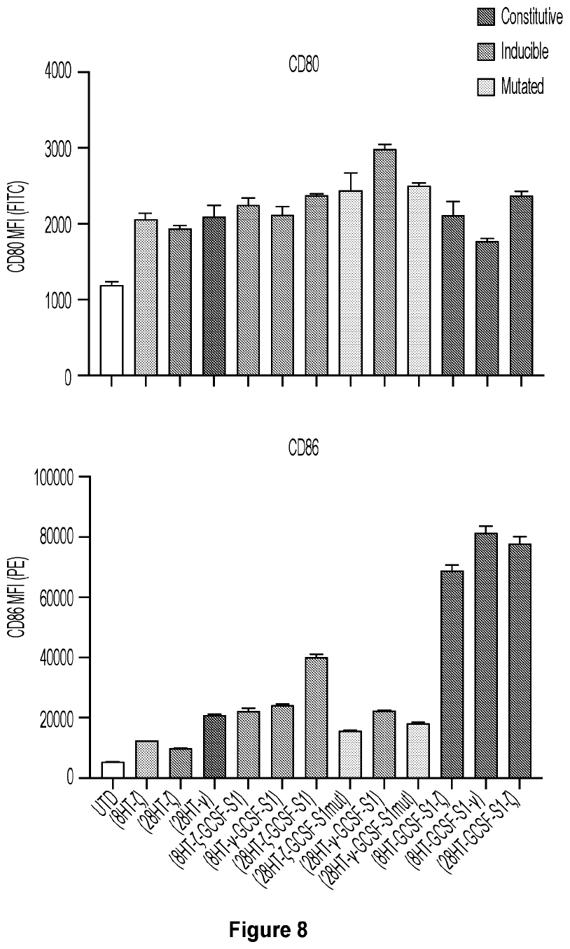

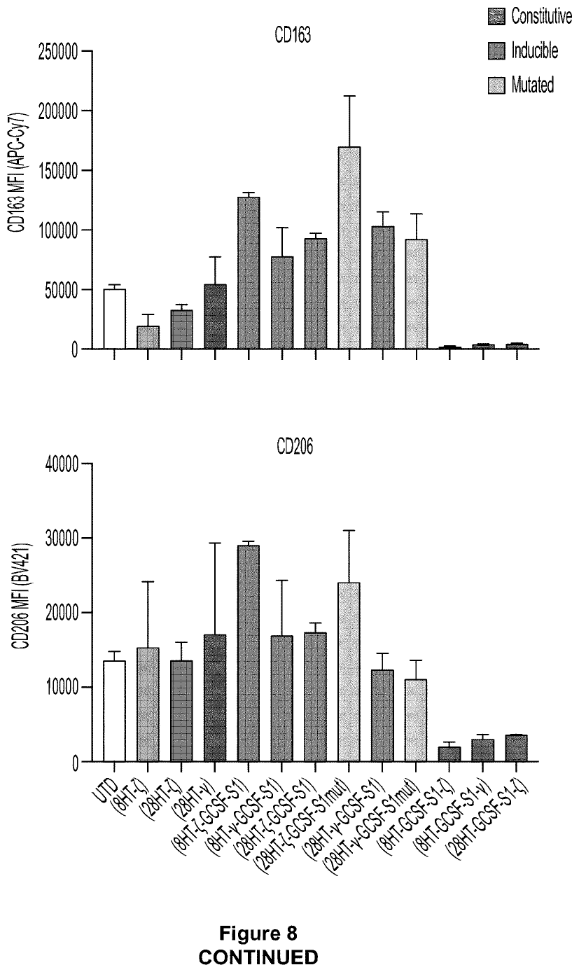

Representative M1/M2 phenotypic marker expression in macrophages expressing constitutive, inducible, and mutated JAK/STAT CAR constructs is shown in FIG. 8. Constitutive JAK/STAT CAR construct expression resulted in macrophage phenotype skewed towards an M¹ phenotype, as characterized by increased M1 marker expression (e.g., CD86) and decreased M2 marker expression (e.g., CD163, CD206), relative to control macrophages and inducible and mutated CAR constructs.

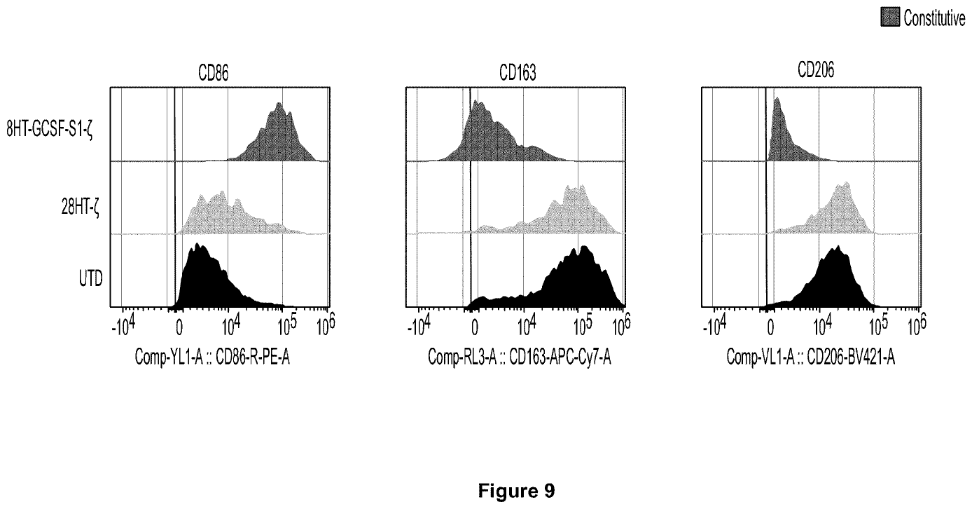

Representative M1/M2 marker phenotypic marker expression via histograms in macrophages expressing a constitutive JAK/STAT CAR construct relative to control is shown in FIG. 9. These data further confirm that macrophages expressing constitutively active JAK/STAT CAR constructs skew towards an M1 phenotype even in the absence of engagement with target antigen.

For assessment of tumor killing function (e.g., cytotoxicity), macrophages were plated at different concentrations in wells of a 96 well flat-bottom dish. Macrophages were incubated for 30-45 minutes at 37° C., followed by addition of tumor cells at different concentrations in equivalent volumes. Tumor cells (e.g., Au565, Panc1, and MDA-MB-468 cells) were engineered prior to the present experiment to express nuclear localized GFP or cytoplasmic GFP. AU565 cells normally overexpress HER2. In some instances, Panc1 tumor cells were engineered prior to the present experiment to express HER2, whereas wild-type Panc1 cells do not express HER2. MDA-MB-468 cells are a HER2-negative breast cancer cell line. Plates were then incubated at room temperature for 15 minutes after addition of tumor cells, followed by 45 minutes incubation at 37° C. GFP expression was then assessed in these co-cultures using an Incucyte.

For all tumor co-cultures, GFP integrated intensity of each well was normalized to integrated intensity at time 0 per well. For data noted as “Change in tumor burden”, the equation used to calculate the value at each time point was: Change in tumor burden=(normalized integrated intensity of co-culture well at time point X)/(normalized integrated intensity of tumor only control at time point X).

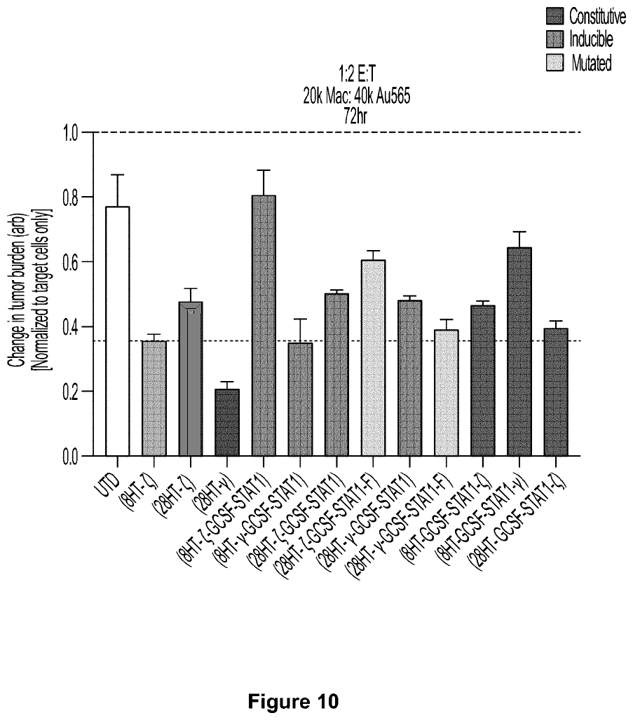

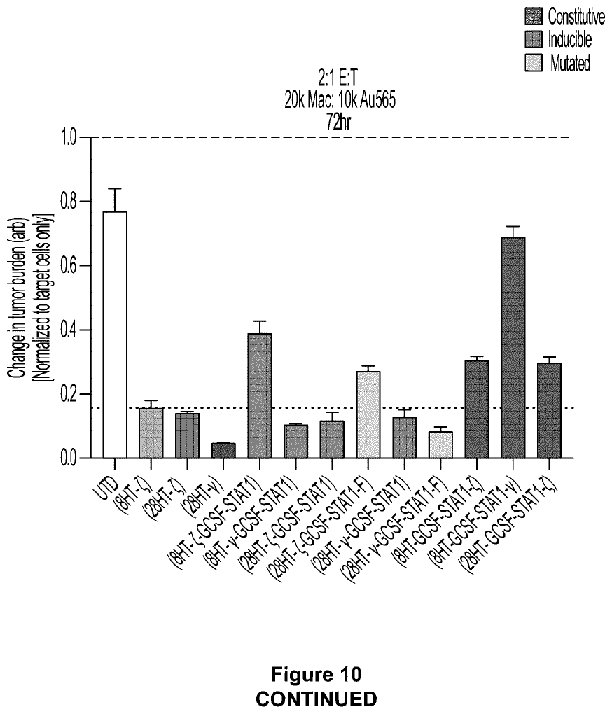

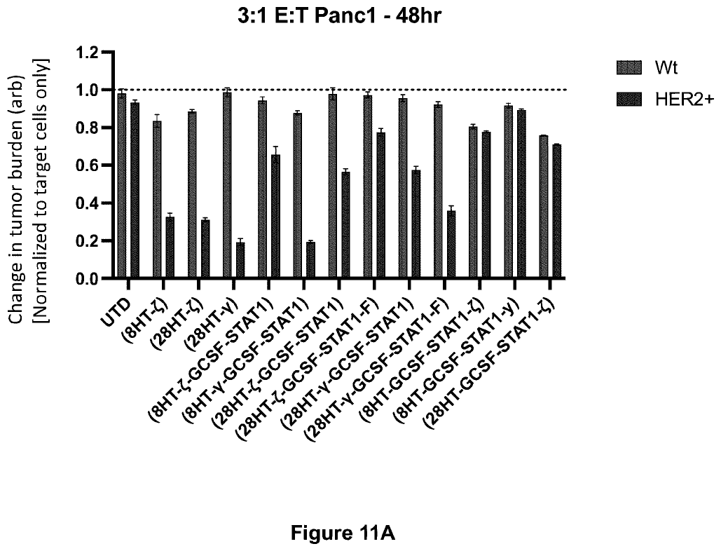

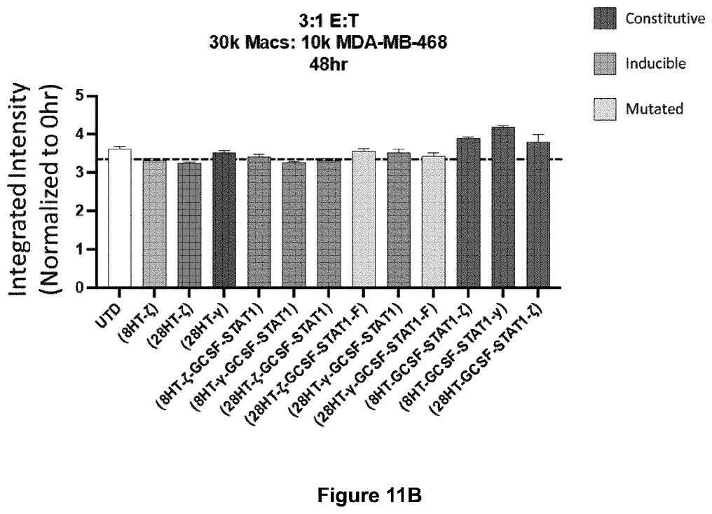

Change in AU565 tumor burden, as measured by GFP integrated intensity normalized to tumor cell only control wells at each time point, is shown in FIG. 10. JAK/STAT CAR constructs demonstrated tumor killing function as compared to untreated control. Change in Panc1 tumor burden, as measured by GFP integrated intensity normalized to tumor cell only control wells at each time point, is shown in FIG. 11A. MDA-MB-468 tumor burden, as measured by GFP intensity relative to time 0 hours of co-culture, is shown in FIG. 11B. Tumor killing function via JAK/STAT CAR constructs was observed to be dependent on the HER2 expression of each cell line, demonstrating antigen specificity.

### Example 3: Inducible, Multi-STAT, JAK/STAT CAR Constructs in Reporter Cell Line

The present Example assesses the effect of multiple STAT domains in inducible JAK/STAT CARS.

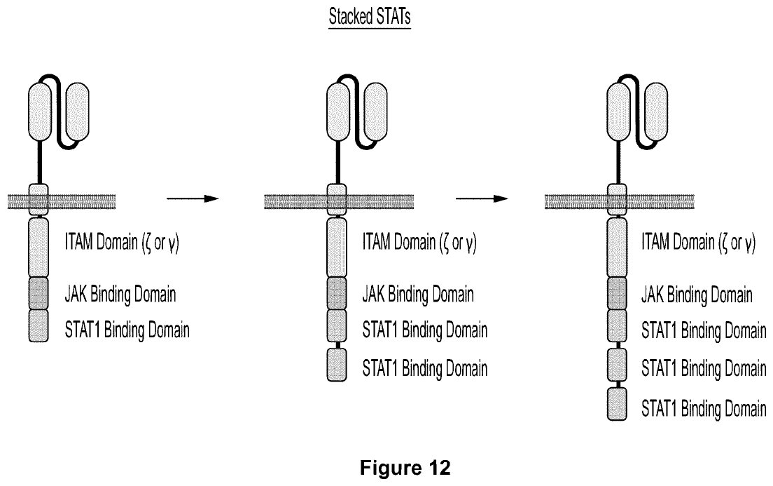

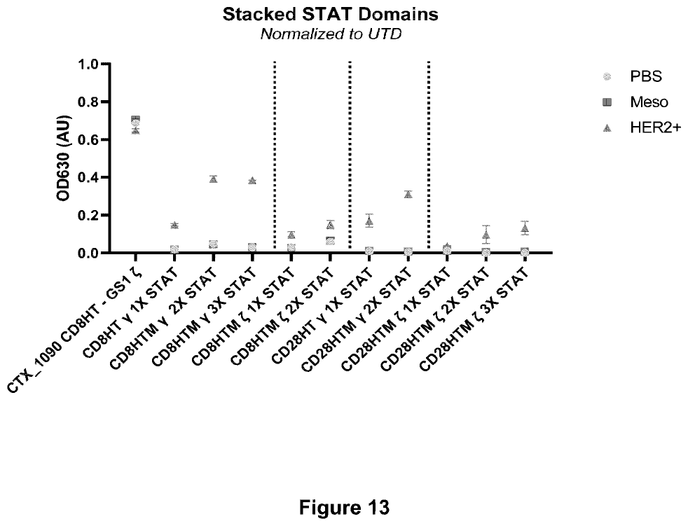

As shown in FIG. 12, CARs comprising an ITAM domain (CD3ζ or FCER1γ), a JAK binding domain, and a STAT1 binding domain were modified to include multiple sequential STAT1 binding domains distal from the cell membrane. To screen JAK-STAT constructs for activity via IFNγ activation, a HEK cell reporter line (HEK Blue IFN-γ, Invivogen) was used. Briefly, 1e6 HEK cells were plated in a 6-well TC plate and incubated overnight at 37° C. and 5% CO₂ in DMEM (10% FBS+1% Pen/Strep). Then, media was exchanged and a transfection mix was added containing 150 μL Optimem, 9 μL Lipofectamine 2000, and 2.5 mg transfer plasmid per reaction. Separately, 96-well plates were coated with either 28 nM HER2-his in PBS, 28 nM MSLN-his in PBS, or PBS alone and kept at 4° C. overnight. 24 hours after transfection, cells were collected and the pre-coated plates were washed with 200 mL PBS 2×. Then, 5e5 cells were plated in triplicate in the coated plates in a total volume of 200 mL per well. As a positive control, untransduced HEK cells were treated with 100 U/mL IFN-γ. After 24- and 48-hours of stimulation, 20 μL of supernatant from the culture was collected and added to 180 μL of QUANTI-Blue solution, incubated for 1.5 hours, and read at OD630. To analyze the data, UTD triplicate values for each condition (PBS, mesothelin (MLSN), HER2) were averaged and subtracted from the individual values of experimental wells of the same condition. As shown in FIG. 13, adding STAT domains to the inducible JAK/STAT CAR architecture did increase HER2-specific STAT signaling output. A signal increase from one to two STAT domains was observed, but no further increase was seen when a third STAT domain was added.

### Example 4: Generation and Function of Inducible, Multi-STAT, JAK/STAT CAR Constructs in Macrophages

The present Example assesses cell viability, CAR expression, tumor killing function, and cytokine secretion in macrophages expressing inducible JAK/STAT CAR constructs comprising one or more STAT domains.

Macrophages were thawed and resuspended to a density of 1e6 cells in TexMacs+10% FBS+1% Pen/Strep+10 ng/mL GM-CSF (complete media; CM). 4e6 cells per sample were aliquoted into tubes and adenovirus particles were added directly to macrophages at specified MOIs, followed by gentle mixing. Macrophages were plated at a density of 1e6 cells/well in 12-well UpCell plates (NUNC). Samples were assayed for CAR expression two days post-transduction.

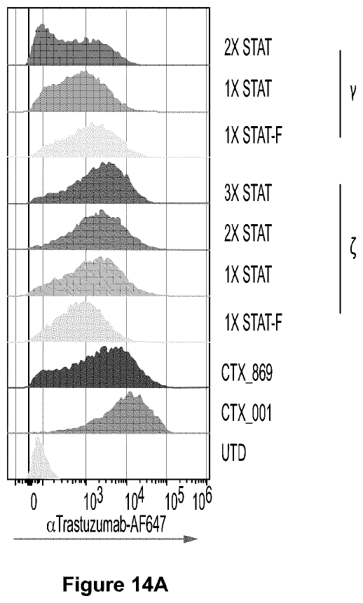

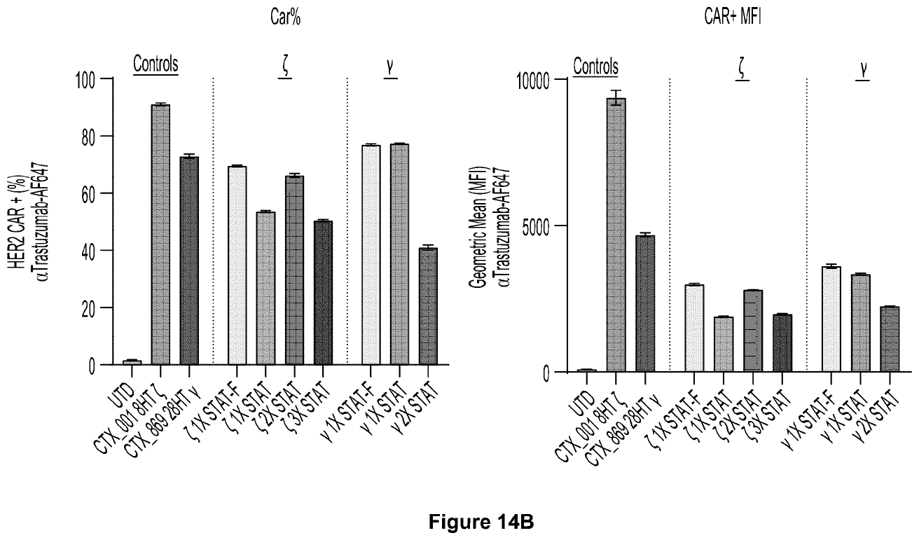

Cell viability and recovery of cells comprising the JAK/STAT CARs are comparable to controls (data not shown). Inducible CAR construct expression (with one or more STAT domains) is shown via histograms in FIG. 14A. Quantification of CAR expression, both in terms of percentage of cells expressing CAR construct and mean fluorescence intensity, is shown in FIG. 14B. JAK/STAT CAR constructs were found to express at the cell surface of macrophages.

For assessment of tumor killing function (e.g., cytotoxicity), macrophages were plated at different concentrations in wells of a 96 well flat-bottom dish. Macrophages were incubated for 30-45 minutes at 37° C., followed by addition of tumor cells at different concentrations in equivalent volumes. AU565 tumor cells were engineered prior to the present experiment to express nuclear localized GFP. AU565 cells normally overexpress HER2. Plates were then incubated at room temperature for 15 minutes after addition of tumor cells, followed by 45 minutes incubation at 37° C. GFP expression was then assessed in these co-cultures using an Incucyte.

For all tumor co-cultures, GFP integrated intensity of each well was normalized to integrated intensity at time 0 per well. For data noted as “Change in tumor burden”, the equation used to calculate the value at each time point was: Change in tumor burden=(normalized integrated intensity of co-culture well at time point X)/(normalized integrated intensity of tumor only control at time point X).

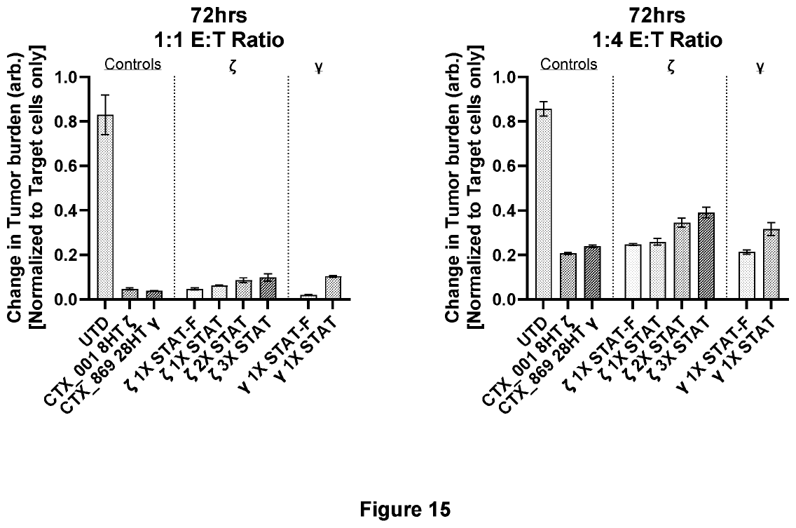

Change in AU565 tumor burden, as measured by GFP integrated intensity normalized to tumor cell only control wells at each time point, is shown in FIG. 15. JAK/STAT1 CAR constructs demonstrated comparable tumor killing function as compared to CTX 869 (CD8L 4D5 (aHer2)_CD28H_CD28TM_FCER1yIC).

For assessment of markers post-antigen stimulation, wells in a 96 well UpCell plate (NUNC) were coated with either PBS or 20 μg/mL HER2-his suspended in PBS and incubated for 2 hours at 37° C. After 2 hours, plate wells were rinsed two times with 200 μL pre-warmed PBS. Macrophages comprising JAK/STAT CARs were plated at 50,000 cells per well in a final volume of 200 μL and incubated overnight at 37° C., 5% CO2. The following day, macrophages were lifted by incubating at 4° C. for 30 minutes. Cells were stained in two separate panels containing Aqua Live/Dead, AF 647-anti-Trastuzumab, AF488-CD80, PerCP-Cy5.5-CD86, AF700-CD206, CD11b-APC-Cy7, CD16 BV421, CD163 PE, CD14 PE-Dazzle 594, HLA-DR PE-Cy7 or Aqua Live/Dead, APC 4-1BBL, AF488-CCR1, PerCP-Cy5.5-PD-L1, AF700-CCR5, CCR7-APC-Cy7, TIM3 BV421, CCR2 PE, CD40 PE-Dazzle 594, Siglec-1 PE-Cy7. Flow cytometry was performed using Attune Cytometer (ThermoFisher).

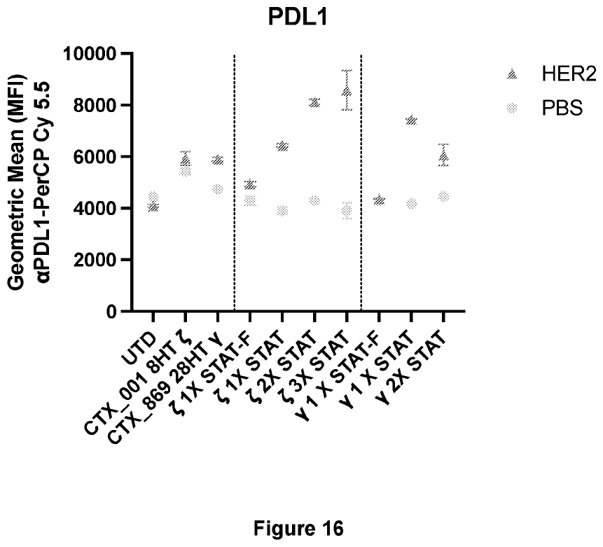

As shown in FIG. 16, macrophages comprising JAK/STAT CARs exhibited antigen-specific and STAT1-specific increased PD-L1 expression. PD-L1 increase is a noted effect of IFN-γ exposure and IFN-γ activation is driven by STAT1 signaling. Observing STAT1-dependent increases in PD-L1 surface expression on the CAR macrophages indicates that the STAT1 pathway is being activated. Further, this is specifically driven by antigen engagement, as PBS condition for all constructs lead to similar expression of PD-L1. The inclusion of the F-mutation in the STAT1 binding domain (i.e., in ζ 1×STAT-F and γ 1x STAT-F), which eliminates the ability of STAT1 to localize to the CAR for phosphorylation, completely abolishes the increase in PD-L1 surface expression. Also, the results indicate that macrophages comprising constructs comprising two or more STAT domains increased antigen-specific and STAT1-specific increased PD-L1 expression relative to macrophages comprising constructs comprising a single STAT domain.

For cytokine secretion assays, antigen (either HER2 or Mesothelin) was resuspended in PBS to a concentration of 28 nM. 100 μL of either solution was added to wells in a 96-well flat bottom plate and incubated at 4° C. for 24 hours. PBS control wells were incubated with PBS only. After incubation, wells were washed 2× with 200 μL PBS, followed by the addition of 50,000 macrophages in a final volume of 200 μL Tex10. Plates were incubated for 24 hours, followed by centrifugation at 300×g for 5 minutes and subsequent removal of 120 μL supernatant. Supernatants were stored at −80° C. Cytokine levels were assessed by Nomic Bio.

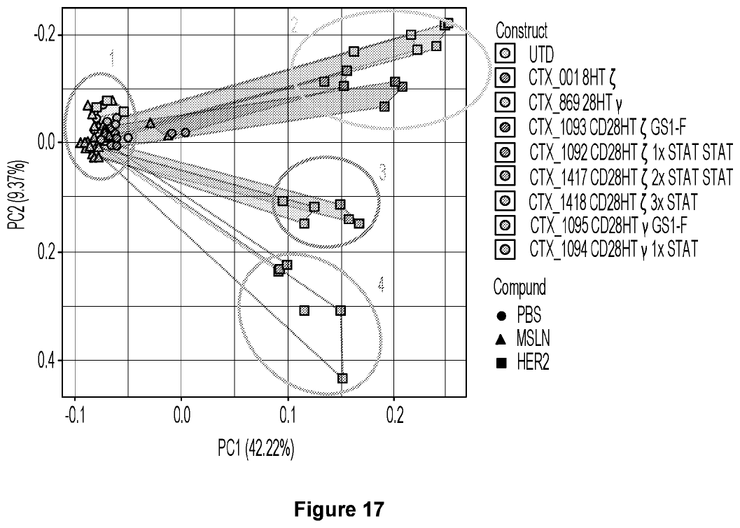

FIG. 17 illustrates an exemplary principal component analysis plot of broad panel cytokine data. The distance between points indicates the difference in cytokine profiles for cells containing the various constructs. The farther two points are from each other, the more different their cytokine profiles are. The data points in section 1 show that results from the constructs all clustered together in the absence of HER2 stimulation (i.e., PBS or mesothelin controls). The data points in section 2 show that results from the CTX 001 and CTX 869 constructs and STAT1 mutant (GS1-F) CAR constructs all clustered together when stimulated with HER2. The data points in section 3 show that results from the 1×STAT constructs clustered together and are similar in pattern and magnitude. Finally, the data points in section 4 show that results from the 2×STAT and 3×STAT constructs cluster together, with 3×STAT constructs displaying a larger difference between the stimulation and lack of stimulation conditions.

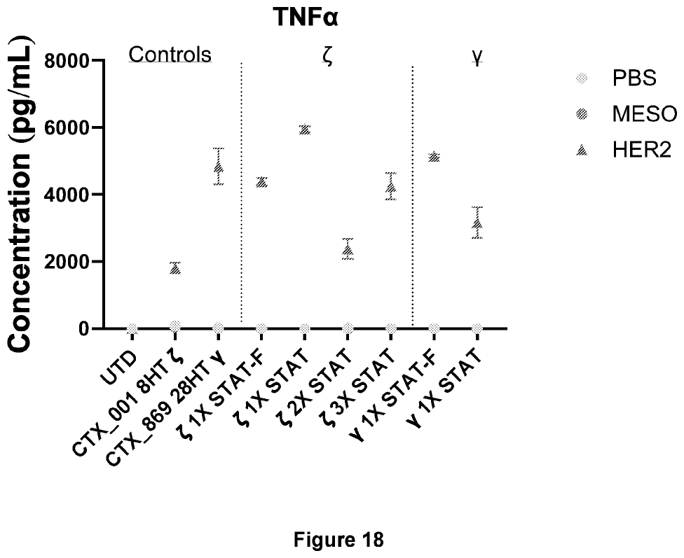

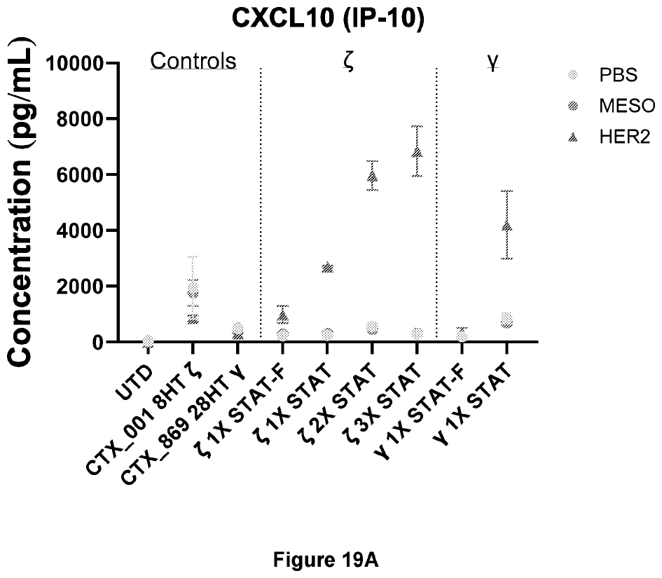

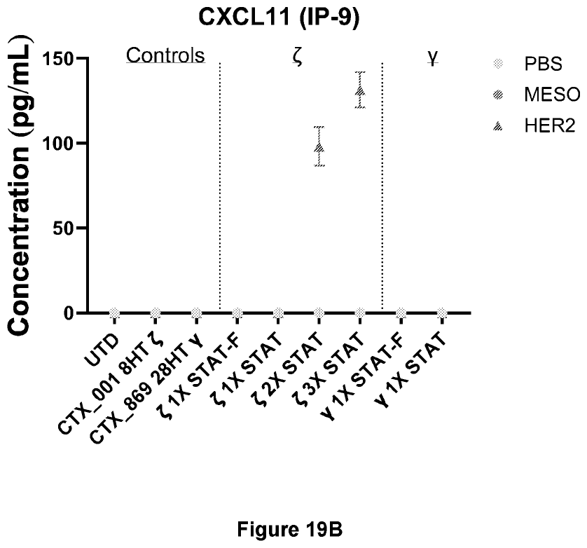

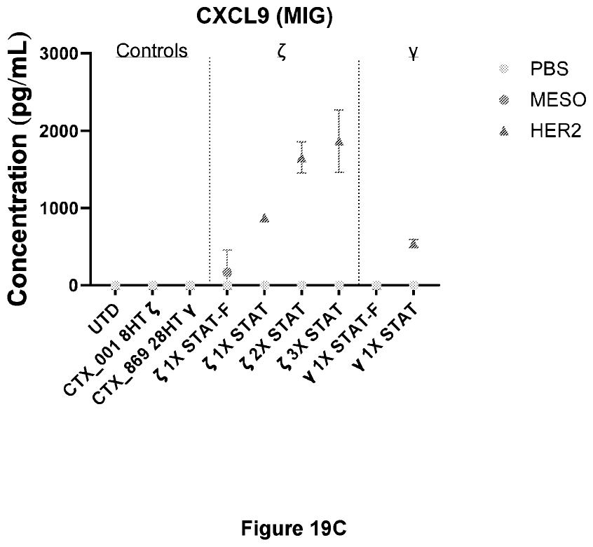

FIG. 18 illustrates exemplary data for a single cytokine (TNFα) taken from the broad panel cytokine analysis. As can been seen, TNFα release from cells comprising inducible JAK/STAT CARs comprising one or more STAT domains was comparable to TNFα release from cells comprising the benchmark CTX_869 CAR. FIGS. 19A-C also illustrate exemplary data for single cytokines taken from the broad panel cytokine analysis. CXCL10 (IP-10), CXCL11 (IP-9) and CXCL9 (MIG) are all are IFNγ-associated cytokines, indicating that the STAT1 signal from the CAR is driving differential cytokine expression relative to the control CARs (CTX_001 and CTX_869). As seen in other assays, mutations to the STAT1 binding domain (STAT-F) eliminate this function.

### EXEMPLARY SEQUENCES

TABLE 2
Exemplary Amino Acid Sequences for Chimeric Antigen Receptors (CARs)

| CAR Description | Amino Acid Sequence |
|---|---|
| CD8aL_4D5(aHer2)_CD8aH_ | MALPVTALLLPLALLLHAARPDIQMTQSPSSLSASVGDRVTITCRASQD |
| CD8aTM_CD3zIC | VNTAVAWYQQKPGKAPKLLIYSASFLYSGVPSRFSGSRSGTDFTLTISS |
| | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCRV |
| | KFSRSADAPAYQQGQNQLYNELNLGRREEYDVLDKRRGRDPEMGGK |
| | PRRKNPQEGLYNELQKDKMAEAYSEIGMKGERRRGKGHDGLYQGLS |
| | TATKDTYDALHMQALPPR (SEQ ID NO: 1) |
| CD8L_4D5(aHer2)_CD28H_ | MALPVTALLLPLALLLHAARPDIQMTQSPSSLSASVGDRVTITCRASQD |
| CD28TM_CD3zIC | VNTAVAWYQQKPGKAPKLLIYSASFLYSGVPSRFSGSRSGTDFTLTISS |
| | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRVKF |
| | SRSADAPAYQQGQNQLYNELNLGRREEYDVLDKRRGRDPEMGGKPR |
| | RKNPQEGLYNELQKDKMAEAYSEIGMKGERRRGKGHDGLYQGLSTA |
| | TKDTYDALHMQALPPR (SEQ ID NO: 2) |
| CD8L_4D5(aHer2)_CD28H_ | MALPVTALLLPLALLLHAARPDIQMTQSPSSLSASVGDRVTITCRASQD |
| CD28TM_FCER1GIC | VNTAVAWYQQKPGKAPKLLIYSASFLYSGVPSRFSGSRSGTDFTLTISS |
| (CTX_869) | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRLKIQ |
| | VRKAAITSYEKSDGVYTGLSTRNQETYETLKHEKPPQ (SEQ ID NO: 3) |
| CD8L_4D5(aHer2)_CD8H_ | MALPVTALLLPLALLLHAARPDIQMTQSPSSLSASVGDRVTITCRASQD |
| CD8TM - CD3z - GCSF- | VNTAVAWYQQKPGKAPKLLIYSASFLYSGVPSRFSGSRSGTDFTLTISS |
| STAT1 ICD | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCRV |
| | KFSRSADAPAYQQGQNQLYNELNLGRREEYDVLDKRRGRDPEMGGK |
| | PRRKNPQEGLYNELQKDKMAEAYSEIGMKGERRRGKGHDGLYQGLS |
| | TATKDTYDALHMQALPPRSPNRKNPLWPSVPDPAHSSLGSWVPTIMEE |
| | DAFQLPGLGTPPITKLTVLEEDEKKPVPWESHNSSETCGLPTLVQTPTSF |
| | GYDKPHVLVDLLVDDSG (SEQ ID NO: 4) |
| CD8L_4D5(aHer2)_CD8H_ | MALPVTALLLPLALLLHAARPDIQMTQSPSSLSASVGDRVTITCRASQD |
| CD8TM - FCER1GIC - | VNTAVAWYQQKPGKAPKLLIYSASFLYSGVPSRFSGSRSGTDFTLTISS |
| GCSF-STAT1 ICD | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCRL |
| | KIQVRKAAITSYEKSDGVYTGLSTRNQETYETLKHEKPPQSPNRKNPL |
| | WPSVPDPAHSSLGSWVPTIMEEDAFQLPGLGTPPITKLTVLEEDEKKPV |
| | PWESHNSSETCGLPTLVQTPTSFGYDKPHVLVDLL VDDSG (SEQ ID |
| | NO: 5) |
| CD8L_4D5(aHer2)_CD8H_ | MALPVTALLLPLALLLHAARPDIQMTQSPSSLSASVGDRVTITCRASQD |
| CD8TM - GCSF-STAT1 | VNTAVAWYQQKPGKAPKLLIYSASFLYSGVPSRFSGSRSGTDFTLTISS |
| ICD - CD3z | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCSPN |
| | RKNPLWPSVPDPAHSSLGSWVPTIMEEDAFQLPGLGTPPITKLTVLEED |
| | EKKPVPWESHNSSETCGLPTLVQTPTSFGYDKPHVLVDLLVDDSGRVK |
| | FSRSADAPAYQQGQNQLYNELNLGRREEYDVLDKRRGRDPEMGGKP |
| | RRKNPQEGLYNELQKDKMAEAYSEIGMKGERRRGKGHDGLYQGLST |
| | ATKDTYDALHMQALPPR (SEQ ID NO: 6) |
| CD8L_4D5(aHer2)_CD8H_ | MALPVTALLLPLALLLHAARPDIQMTQSPSSLSASVGDRVTITCRASQD |
| CD8TM - GCSF-STAT1 | VNTAVAWYQQKPGKAPKLLIYSASFLYSGVPSRFSGSRSGTDFTLTISS |
| ICD - FCER1GIC | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCSPN |
| | RKNPLWPSVPDPAHSSLGSWVPTIMEEDAFQLPGLGTPPITKLTVLEED |
| | EKKPVPWESHNSSETCGLPTLVQTPTSFGYDKPHVLVDLLVDDSGRLK |
| | IQVRKAAITSYEKSDGVYTGLSTRNQETYETLKHEKPPQ (SEQ ID NO: |
| | 7) |
| CD8L 4D5(aHer2)_CD28H_ | MALPVTALLLPLALLLHAARPDIQMTQSPSSLSASVGDRVTITCRASQD |
| CD28TM - CD3z - GCSF- | VNTAVAWYQQKPGKAPKLLIYSASFLYSGVPSRFSGSRSGTDFTLTISS |
| STAT1 ICD | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRVKF |
| | SRSADAPAYQQGQNQLYNELNLGRREEYDVLDKRRGRDPEMGGKPR |
| | RKNPQEGLYNELQKDKMAEAYSEIGMKGERRRGKGHDGLYQGLSTA |
| | TKDTYDALHMQALPPRSPNRKNPLWPSVPDPAHSSLGSWVPTIMEED |
| | AFQLPGLGTPPITKLTVLEEDEKKPVPWESHNSSETCGLPTLVQTPTSFG |
| | YDKPHVLVDLLVDDSG (SEQ ID NO: 8) |
| CD8L_4D5(aHer2)_CD28H_ | MALPVTALLLPLALLLHAARPDIQMTQSPSSLSASVGDRVTITCRASQD |
| CD28TM - CD3z - GCSF- | VNTAVAWYQQKPGKAPKLLIYSASFLYSGVPSRFSGSRSGTDFTLTISS |
| STAT1 ICD-F | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRVKF |
| | SRSADAPAYQQGQNQLYNELNLGRREEYDVLDKRRGRDPEMGGKPR |
| | RKNPQEGLYNELQKDKMAEAYSEIGMKGERRRGKGHDGLYQGLSTA |
| | TKDTYDALHMQALPPRSPNRKNPLWPSVPDPAHSSLGSWVPTIMEED |
| | AFQLPGLGTPPITKLTVLEEDEKKPVPWESHNSSETCGLPTLVQTPTSFG |
| | FDKPHVLVDLL VDDSG (SEQ ID NO: 9) |
| CD8L_4D5(aHer2)_CD28H_ | MALPVTALLLPLALLLHAARPDIQMTQSPSSLSASVGDRVTITCRASQD |
| CD28TM - FCER1GIC - | VNTAVAWYQQKPGKAPKLLIYSASFLYSGVPSRFSGSRSGTDFTLTISS |
| GCSF-STAT1 ICD | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRLKIQ |
| | VRKAAITSYEKSDGVYTGLSTRNQETYETLKHEKPPQSPNRKNPLWPS |
| | VPDPAHSSLGSWVPTIMEEDAFQLPGLGTPPITKLTVLEEDEKKPVPWE |
| | SHNSSETCGLPTLVQTPTSFGYDKPHVLVDLLVDDSG (SEQ ID NO: 10) |
| CD8L_4D5(aHer2)_CD28H_ | MALPVTALLLPLALLLHAARPDIQMTQSPSSLSASVGDRVTITCRASQD |
| CD28TM - FCER1GIC - | VNTAVAWYQQKPGKAPKLLIYSASFLYSGVPSRFSGSRSGTDFTLTISS |
| GCSF-STAT1-F | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRLKIQ |
| | VRKAAITSYEKSDGVYTGLSTRNQETYETLKHEKPPQSPNRKNPLWPS |
| | VPDPAHSSLGSWVPTIMEEDAFQLPGLGTPPITKLTVLEEDEKKPVPWE |
| | SHNSSETCGLPTLVQTPTSFGFDKPHVLVDLLVDDSG (SEQ ID NO: 11) |
| CD8L_4D5(aHer2)_CD28H_ | MALPVTALLLPLALLLHAARPDIQMTQSPSSLSASVGDRVTITCRASQD |
| CD28TM - GCSF-STAT1 | VNTAVAWYQQKPGKAPKLLIYSASFLYSGVPSRFSGSRSGTDFTLTISS |
| ICD - CD3z | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVSPNR |
| | KNPLWPSVPDPAHSSLGSWVPTIMEEDAFQLPGLGTPPITKLTVLEEDE |
| | KKPVPWESHNSSETCGLPTLVQTPTSFGYDKPHVLVDLL VDDSGRVKF |
| | SRSADAPAYQQGQNQLYNELNLGRREEYDVLDKRRGRDPEMGGKPR |
| | RKNPQEGLYNELQKDKMAEAYSEIGMKGERRRGKGHDGLYQGLSTA |
| | TKDTYDALHMQALPPR (SEQ ID NO: 12) |
| CD8L_4D5(aHer2)_CD28H_ | MALPVTALLLPLALLLHAARPDIQMTQSPSSLSASVGDRVTITCRASQD |
| CD28TM - GCSF-STAT1 | VNTAVAWYQQKPGKAPKLLIYSASFLYSGVPSRFSGSRSGTDFTLTISS |
| ICD - FCER1GIC | LQPEDFATYYCQQHYTTPPTFGQGTKVEIKRTGSTSGSGKPGSGEGSEV |
| | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVSPNR |
| | KNPLWPSVPDPAHSSLGSWVPTIMEEDAFQLPGLGTPPITKLTVLEEDE |
| | KKPVPWESHNSSETCGLPTLVQTPTSFGYDKPHVLVDLL VDDSGRLKI |
| | QVRKAAITSYEKSDGVYTGLSTRNQETYETLKHEKPPQ (SEQ ID NO: |
| | 13) |
| CD8L_4D5(aHer2)_CD8H_ | MALPVTALLLPLALLLHAARPDIQMTQSPSSLSASVGDRVTITCRASQD |
| CD8TM - CD3z - GCSF- | VNTAVAWYQQKPGKAPKLLIYSASFLYSGVPSRFSGSRSGTDFTLTISS |
| STAT1 ICD - (G4S)*3 | LQPEDFATYYCQQHYTTPPTFGQGTKVEIKRTGSTSGSGKPGSGEGSEV |
| Linker - STAT1 ICD | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCRV |
| | KFSRSADAPAYQQGQNQLYNELNLGRREEYDVLDKRRGRDPEMGGK |
| | PRRKNPQEGLYNELQKDKMAEAYSEIGMKGERRRGKGHDGLYQGLS |
| | TATKDTYDALHMQALPPRSPNRKNPLWPSVPDPAHSSLGSWVPTIMEE |
| | DAFQLPGLGTPPITKLTVLEEDEKKPVPWESHNSSETCGLPTLVQTPTSF |
| | GYDKPHVLVDLLVDDSGGGGGSGGGGSGGGGSPTSFGYDKPHVLVD |
| | LLVDDSG (SEQ ID NO: 150) |
| CD8L_4D5(aHer2)_CD8H_ | MALPVTALLLPLALLLHAARPDIQMTQSPSSLSASVGDRVTITCRASQD |
| CD8TM - CD3z - GCSF- | VNTAVAWYQQKPGKAPKLLIYSASFLYSGVPSRFSGSRSGTDFTLTISS |
| STAT1 ICD - (G4S)*3 | LQPEDFATYYCQQHYTTPPTFGQGTKVEIKRTGSTSGSGKPGSGEGSEV |
| Linker - STAT1 ICD - | QLVESGGGLVQPGGSLRLSCAASGFNIKDTYIHWVRQAPGKGLEWVA |
| (G4S)*3 Linker - STAT1 | RIYPTNGYTRYADSVKGRFTISADTSKNTAYLQMNSLRAEDTAVYYCS |
| ICD | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCRV |
| | KFSRSADAPAYQQGQNQLYNELNLGRREEYDVLDKRRGRDPEMGGK |
| | PRRKNPQEGLYNELQKDKMAEAYSEIGMKGERRRGKGHDGLYQGLS |
| | TATKDTYDALHMQALPPRSPNRKNPLWPSVPDPAHSSLGSWVPTIMEE |
| | DAFQLPGLGTPPITKLTVLEEDEKKPVPWESHNSSETCGLPTLVQTPTSF |
| | GYDKPHVLVDLLVDDSGGGGGSGGGGSGGGGSPTSFGYDKPHVLVD |
| | LLVDDSGGGGGSGGGGSGGGGSPTSFGYDKPHVLVDLLVDDSG (SEQ |
| | ID NO: 151) |
| CD8L_4D5(aHer2)_CD8H_ | MALPVTALLLPLALLLHAARPDIQMTQSPSSLSASVGDRVTITCRASQD |
| CD8TM - FCER1GIC - | VNTAVAWYQQKPGKAPKLLIYSASFLYSGVPSRFSGSRSGTDFTLTISS |
| GCSF-STAT1 ICD - | LQPEDFATYYCQQHYTTPPTFGQGTKVEIKRTGSTSGSGKPGSGEGSEV |
| (G4S)*3 Linker - STAT1 | QLVESGGGLVQPGGSLRLSCAASGFNIKDTYIHWVRQAPGKGLEWVA |
| ICD | RIYPTNGYTRYADSVKGRFTISADTSKNTAYLQMNSLRAEDTAVYYCS |
| | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCRL |
| | KIQVRKAAITSYEKSDGVYTGLSTRNQETYETLKHEKPPQSPNRKNPL |
| | WPSVPDPAHSSLGSWVPTIMEEDAFQLPGLGTPPITKLTVLEEDEKKPV |
| | PWESHNSSETCGLPTLVQTPTSFGYDKPHVLVDLLVDDSGGGGGSGG |
| | GGSGGGGSPTSFGYDKPHVLVDLLVDDSG (SEQ ID NO: 152) |
| CD8L_4D5(aHer2)_CD8H_ | MALPVTALLLPLALLLHAARPDIQMTQSPSSLSASVGDRVTITCRASQD |
| CD8TM - FCER1GIC - | VNTAVAWYQQKPGKAPKLLIYSASFLYSGVPSRFSGSRSGTDFTLTISS |
| GCSF-STAT1 ICD - | LQPEDFATYYCQQHYTTPPTFGQGTKVEIKRTGSTSGSGKPGSGEGSEV |
| (G4S)*3 Linker - STAT1 | QLVESGGGLVQPGGSLRLSCAASGFNIKDTYIHWVRQAPGKGLEWVA |
| ICD - (G4S)*3 Linker - | RIYPTNGYTRYADSVKGRFTISADTSKNTAYLQMNSLRAEDTAVYYCS |
| STAT1 ICD | RWGGDGFYAMDVWGQGTLVTVSSSGTTTPAPRPPTPAPTIASQPLSLR |
| | PEACRPAAGGAVHTRGLDFACDIYIWAPLAGTCGVLLLSLVITLYCRL |
| | KIQVRKAAITSYEKSDGVYTGLSTRNQETYETLKHEKPPQSPNRKNPL |
| | WPSVPDPAHSSLGSWVPTIMEEDAFQLPGLGTPPITKLTVLEEDEKKPV |
| | PWESHNSSETCGLPTLVQTPTSFGYDKPHVLVDLLVDDSGGGGGSGG |
| | GGSGGGGSPTSFGYDKPHVLVDLLVDDSGGGGGSGGGGSGGGGSPTS |
| | FGYDKPHVLVDLLVDDSG (SEQ ID NO: 153) |
| CD8L_4D5(aHer2)_CD28H_ | MALPVTALLLPLALLLHAARPDIQMTQSPSSLSASVGDRVTITCRASQD |
| CD28TM - CD3z - GCSF- | VNTAVAWYQQKPGKAPKLLIYSASFLYSGVPSRFSGSRSGTDFTLTISS |
| STAT1 ICD - (G4S)*3 | LQPEDFATYYCQQHYTTPPTFGQGTKVEIKRTGSTSGSGKPGSGEGSEV |
| Linker - STAT1 ICD | QLVESGGGLVQPGGSLRLSCAASGFNIKDTYIHWVRQAPGKGLEWVA |
| | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRVKF |
| | SRSADAPAYQQGQNQLYNELNLGRREEYDVLDKRRGRDPEMGGKPR |
| | RKNPQEGLYNELQKDKMAEAYSEIGMKGERRRGKGHDGLYQGLSTA |
| | TKDTYDALHMQALPPRSPNRKNPLWPSVPDPAHSSLGSWVPTIMEED |
| | AFQLPGLGTPPITKLTVLEEDEKKPVPWESHNSSETCGLPTLVQTPTSFG |
| | YDKPHVLVDLLVDDSGGGGGSGGGGSGGGGSPTSFGYDKPHVLVDLL |
| | VDDSG (SEQ ID NO: 154) |
| CD8L_4D5(aHer2)_CD28H_ | MALPVTALLLPLALLLHAARPDIQMTQSPSSLSASVGDRVTITCRASQD |
| CD28TM - CD3z - GCSF- | VNTAVAWYQQKPGKAPKLLIYSASFLYSGVPSRFSGSRSGTDFTLTISS |
| STAT1 ICD - (G4S)*3 | LQPEDFATYYCQQHYTTPPTFGQGTKVEIKRTGSTSGSGKPGSGEGSEV |
| Linker - STAT1 ICD - | QLVESGGGLVQPGGSLRLSCAASGFNIKDTYIHWVRQAPGKGLEWVA |
| (G4S)*3 Linker - STAT1 | RIYPTNGYTRYADSVKGRFTISADTSKNTAYLQMNSLRAEDTAVYYCS |
| ICD | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRVKF |
| | SRSADAPAYQQGQNQLYNELNLGRREEYDVLDKRRGRDPEMGGKPR |
| | RKNPQEGLYNELQKDKMAEAYSEIGMKGERRRGKGHDGLYQGLSTA |
| | TKDTYDALHMQALPPRSPNRKNPLWPSVPDPAHSSLGSWVPTIMEED |
| | AFQLPGLGTPPITKLTVLEEDEKKPVPWESHNSSETCGLPTLVQTPTSFG |
| | YDKPHVLVDLLVDDSGGGGGSGGGGSGGGGSPTSFGYDKPHVLVDLL |
| | VDDSGGGGGSGGGGSGGGGSPTSFGYDKPHVLVDLLVDDSG (SEQ ID |
| | NO: 155) |
| CD8L_4D5(aHer2)_CD28H_ | MALPVTALLLPLALLLHAARPDIQMTQSPSSLSASVGDRVTITCRASQD |
| CD28TM - FCER1GIC - | VNTAVAWYQQKPGKAPKLLIYSASFLYSGVPSRFSGSRSGTDFTLTISS |
| GCSF-STAT1 ICD - | LQPEDFATYYCQQHYTTPPTFGQGTKVEIKRTGSTSGSGKPGSGEGSEV |
| (G4S)*3 Linker - STAT1 | QLVESGGGLVQPGGSLRLSCAASGFNIKDTYIHWVRQAPGKGLEWVA |
| ICD | RIYPTNGYTRYADSVKGRFTISADTSKNTAYLQMNSLRAEDTAVYYCS |
| | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRLKIQ |
| | VRKAAITSYEKSDGVYTGLSTRNQETYETLKHEKPPQSPNRKNPLWPS |
| | VPDPAHSSLGSWVPTIMEEDAFQLPGLGTPPITKLTVLEEDEKKPVPWE |
| | SHNSSETCGLPTLVQTPTSFGYDKPHVLVDLLVDDSGGGGGSGGGGSG |
| | GGGSPTSFGYDKPHVLVDLLVDDSG (SEQ ID NO: 156) |
| CD8L_4D5(aHer2)_CD28H_ | MALPVTALLLPLALLLHAARPDIQMTQSPSSLSASVGDRVTITCRASQD |
| CD28TM - FCER1GIC - | VNTAVAWYQQKPGKAPKLLIYSASFLYSGVPSRFSGSRSGTDFTLTISS |
| GCSF-STAT1 ICD - | LQPEDFATYYCQQHYTTPPTFGQGTKVEIKRTGSTSGSGKPGSGEGSEV |
| (G4S)*3 Linker - STAT1 | QLVESGGGLVQPGGSLRLSCAASGFNIKDTYIHWVRQAPGKGLEWVA |
| ICD - (G4S)*3 Linker - | RIYPTNGYTRYADSVKGRFTISADTSKNTAYLQMNSLRAEDTAVYYCS |
| STAT1 ICD | RWGGDGFYAMDVWGQGTLVTVSSSGIEVMYPPPYLDNEKSNGTIIHV |
| | KGKHLCPSPLFPGPSKPFWVLVVVGGVLACYSLLVTVAFIIFWVRLKIQ |
| | VRKAAITSYEKSDGVYTGLSTRNQETYETLKHEKPPQSPNRKNPLWPS |
| | VPDPAHSSLGSWVPTIMEEDAFQLPGLGTPPITKLTVLEEDEKKPVPWE |
| | SHNSSETCGLPTLVQTPTSFGYDKPHVLVDLLVDDSGGGGGSGGGGSG |
| | GGGSPTSFGYDKPHVLVDLLVDDSGGGGGSGGGGSGGGGSPTSFGYD |
| | KPHVLVDLLVDDSG (SEQ ID NO: 157) |

TABLE 3
Exemplary Nucleotide Sequences for Chimeric Antigen Receptors (CARs)

| CAR Description | Amino Acid Sequence |
|---|---|
| CD8aL_4D5(aHer2)_CD8aH_ | ATGGCCTTACCAGTGACCGCCTTGCTCCTGCCGCTGGCCTTGCTGCT |
| CD8aTM_CD3zIC | CCACGCCGCCAGGCCGGACATTCAAATGACACAGAGTCCCTCATCC |
| | CTCAGTGCCAGCGTGGGCGATCGGGTGACTATAACCTGCAGAGCTT |
| | CTCAGGACGTGAATACCGCTGTGGCGTGGTACCAGCAGAAGCCAG |
| | GCAAAGCGCCTAAGCTTCTCATTTATAGTGCCAGCTTCCTGTACTCA |
| | GGTGTTCCGTCTCGCTTTTCTGGAAGTAGAAGTGGGACCGATTTCAC |
| | ATTGACGATCAGCAGCTTGCAGCCCGAAGATTTCGCCACCTACTAC |
| | TGTCAGCAGCACTACACTACCCCACCGACATTTGGTCAAGGCACAA |
| | AAGTAGAGATTAAACGCACTGGTTCCACCAGCGGGAGCGGGAAAC |
| | CCGGCTCTGGGGAGGGGAGCGAGGTCCAGCTGGTGGAATCCGGGG |
| | GTGGTCTTGTGCAGCCAGGAGGATCCTTGAGGTTGTCCTGCGCCGC |
| | AAGCGGCTTTAACATCAAAGATACATACATCCATTGGGTCCGACAG |
| | GCCCCTGGAAAGGGCCTGGAGTGGGTCGCCCGGATCTACCCAACTA |
| | ACGGGTACACTCGCTACGCTGATAGCGTCAAGGGTCGGTTTACTAT |
| | TTCTGCCGACACCTCAAAAAACACAGCCTACCTCCAGATGAACTCT |
| | CTCAGAGCTGAGGATACAGCCGTGTACTATTGCAGCCGGTGGGGAG |
| | GCGACGGGTTCTACGCTATGGATGTGTGGGGGCAGGGCACACTGGT |
| | CACCGTGAGCTCATCCGGAACCACGACGCCAGCGCCGCGACCACC |
| | AACACCGGCGCCCACCATCGCGTCGCAGCCCCTGTCCCTGCGCCCA |
| | GAGGCGTGCCGGCCAGCGGGGGGGGCGCAGTGCACACGAGGGGG |
| | CTGGACTTCGCCTGTGATATCTACATCTGGGCGCCCTTGGCCGGGA |
| | CTTGTGGGGTCCTTCTCCTGTCACTGGTTATCACCCTTTACTGCAGA |
| | GTGAAGTTCAGCAGGAGCGCAGACGCCCCCGCGTACCAGCAGGGC |
| | CAGAACCAGCTCTATAACGAGCTCAATCTAGGACGAAGAGAGGAG |
| | TACGATGTTTTGGACAAGAGACGTGGCCGGGACCCTGAGATGGGG |
| | GGAAAGCCGAGAAGGAAGAACCCTCAGGAAGGCCTGTACAATGAA |
| | CTGCAGAAAGATAAGATGGCGGAGGCCTACAGTGAGATTGGGATG |
| | AAAGGCGAGCGCCGGAGGGGCAAGGGGCACGATGGCCTTTACCAG |
| | GGTCTCAGTACAGCCACCAAGGACACCTACGACGCCCTTCACATGC |
| | AGGCCCTGCCCCCTCGCTAA (SEQ ID NO: 14) |
| CD8L_4D5(aHer2)_CD28H_ | ATGGCCTTACCAGTGACCGCCTTGCTCCTGCCGCTGGCCTTGCTGCT |
| CD28TM_CD3zIC | CCACGCCGCCAGGCCGGACATTCAAATGACACAGAGTCCCTCATCC |
| | CTCAGTGCCAGCGTGGGCGATCGGGTGACTATAACCTGCAGAGCTT |
| | CTCAGGACGTGAATACCGCTGTGGCGTGGTACCAGCAGAAGCCAG |
| | GCAAAGCGCCTAAGCTTCTCATTTATAGTGCCAGCTTCCTGTACTCA |
| | GGTGTTCCGTCTCGCTTTTCTGGAAGTAGAAGTGGGACCGATTTCAC |
| | ATTGACGATCAGCAGCTTGCAGCCCGAAGATTTCGCCACCTACTAC |
| | TGTCAGCAGCACTACACTACCCCACCGACATTTGGTCAAGGCACAA |
| | AAGTAGAGATTAAACGCACTGGTTCCACCAGCGGGAGCGGGAAAC |
| | CCGGCTCTGGGGAGGGGAGCGAGGTCCAGCTGGTGGAATCCGGGG |
| | GTGGTCTTGTGCAGCCAGGAGGATCCTTGAGGTTGTCCTGCGCCGC |
| | AAGCGGCTTTAACATCAAAGATACATACATCCATTGGGTCCGACAG |
| | GCCCCTGGAAAGGGCCTGGAGTGGGTCGCCCGGATCTACCCAACTA |
| | ACGGGTACACTCGCTACGCTGATAGCGTCAAGGGTCGGTTTACTAT |
| | TTCTGCCGACACCTCAAAAAACACAGCCTACCTCCAGATGAACTCT |
| | CTCAGAGCTGAGGATACAGCCGTGTACTATTGCAGCCGGTGGGGAG |
| | GCGACGGGTTCTACGCTATGGATGTGTGGGGGCAGGGCACACTGGT |
| | CACCGTGAGCTCATCCGGAATTGAAGTGATGTATCCGCCGCCGTAT |
| | CTGGATAACGAAAAAAGCAACGGCACCATTATTCATGTGAAAGGC |
| | AAACATCTGTGCCCGAGCCCGCTGTTTCCGGGCCCGAGCAAACCGT |
| | TTTGGGTGCTGGTGGTGGTGGGCGGCGTGCTGGCGTGCTATAGCCT |
| | GCTGGTGACCGTGGCGTTTATTATTTTTTGGGTGAGAGTGAAGTTCA |
| | GCAGGAGCGCAGACGCCCCCGCGTACCAGCAGGGCCAGAACCAGC |
| | TCTATAACGAGCTCAATCTAGGACGAAGAGAGGAGTACGATGTTTT |
| | GGACAAGAGACGTGGCCGGGACCCTGAGATGGGGGGAAAGCCGAG |
| | AAGGAAGAACCCTCAGGAAGGCCTGTACAATGAACTGCAGAAAGA |
| | TAAGATGGCGGAGGCCTACAGTGAGATTGGGATGAAAGGCGAGCG |
| | CCGGAGGGGCAAGGGGCACGATGGCCTTTACCAGGGTCTCAGTAC |
| | AGCCACCAAGGACACCTACGACGCCCTTCACATGCAGGCCCTGCCC |
| | CCTCGCTAA (SEQ ID NO: 15) |
| CD8L_4D5(aHer2)_CD28H_ | ATGGCACTTCCCGTCACAGCACTTCTGCTCCCGCTGGCCCTGCTCCT |
| CD28TM_FCER1GIC | GCATGCCGCACGCCCTGACATCCAGATGACCCAGAGCCCATCCTCT |
| | CTGAGCGCGTCCGTGGGCGACAGAGTGACCATCACCTGTAGAGCCT |
| | CCCAGGACGTCAATACCGCTGTGGCCTGGTACCAACAGAAGCCAGG |
| | AAAGGCCCCGAAGCTGCTCATCTACAGCGCCTCATTCCTGTACTCC |
| | GGCGTGCCTAGCAGATTCTCTGGTAGTCGGTCCGGCACAGATTTCA |
| | CCTTGACAATCAGCTCTTTGCAACCTGAGGACTTTGCTACCTATTAC |
| | TGCCAACAGCACTACACTACACCGCCCACGTTTGGTCAAGGCACCA |
| | AAGTGGAGATCAAGCGGACTGGTAGCACAAGTGGTTCAGGTAAGC |
| | CTGGAAGCGGAGAAGGTTCTGAGGTGCAACTCGTTGAATCTGGCGG |
| | TGGCCTGGTCCAGCCTGGCGGTTCCCTGCGCCTTTCTTGTGCTGCGT |
| | CCGGTTTCAACATCAAGGACACCTACATCCATTGGGTTCGTCAGGC |
| | ACCCGGTAAGGGACTCGAGTGGGTAGCCCGCATTTATCCCACAAAC |
| | GGATACACTAGATACGCTGATTCCGTCAAGGGCCGTTTTACCATCA |
| | GTGCCGACACATCCAAGAATACTGCCTACCTTCAGATGAACAGCCT |
| | CCGCGCCGAAGACACCGCGGTGTATTACTGTTCCCGCTGGGGAGGC |
| | GATGGATTCTACGCAATGGACGTCTGGGGCCAGGGGACCCTCGTGA |
| | CAGTCTCCTCAAGCGGCATCGAGGTCATGTACCCGCCACCCTACCT |
| | GGACAACGAGAAGTCCAACGGGACAATTATCCATGTTAAGGGCAA |
| | GCACCTGTGCCCTAGCCCCCTGTTCCCCGGTCCCTCCAAACCGTTCT |
| | GGGTCCTGGTAGTGGTTGGCGGTGTGTTGGCTTGCTACTCTCTCCTG |
| | GTTACCGTCGCCTTCATCATTTTCTGGGTACGTCTGAAGATTCAGGT |
| | GCGTAAAGCTGCAATCACCTCATATGAGAAATCCGACGGGGTGTAC |
| | ACCGGCCTGTCCACAAGGAACCAGGAGACCTACGAGACCCTCAAG |
| | CACGAGAAGCCTCCACAGTAA (SEQ ID NO: 16) |
| CD8L_4D5(aHer2)_CD8H_ | ATGGCCCTGCCTGTAACAGCCCTCCTGCTCCCACTGGCTCTGCTCCT |
| CD8TM - CD3z - GCSF- | GCATGCGGCCAGGCCTGACATCCAAATGACACAAAGCCCCTCTAGC |
| STAT1 ICD | TTGTCCGCAAGTGTCGGTGATAGGGTGACGATTACTTGCCGTGCCT |
| | CCCAGGACGTCAACACCGCTGTTGCTTGGTACCAACAGAAGCCGGG |
| | CAAAGCGCCGAAACTCCTGATCTACTCTGCAAGTTTCCTGTACAGC |
| | GGTGTCCCTAGTCGGTTCTCTGGCAGCCGCTCAGGAACTGATTTCAC |
| | ACTGACCATCAGTTCCCTGCAGCCTGAGGATTTCGCTACCTATTACT |
| | GCCAACAGCATTACACTACCCCACCCACCTTCGGACAAGGAACCAA |
| | AGTTGAAATCAAGCGCACCGGCAGTACGTCTGGCTCTGGGAAGCCC |
| | GGAAGCGGGGAGGGCAGCGAGGTCCAGTTGGTCGAGTCTGGGGGC |
| | GGGCTGGTGCAGCCTGGGGGATCCCTGAGATTGTCATGCGCCGCAA |
| | GCGGCTTTAACATCAAAGACACTTACATCCATTGGGTGCGGCAAGC |
| | TCCCGGGAAGGGCCTGGAGTGGGTAGCACGCATCTATCCCACCAAT |
| | GGCTACACCCGCTACGCCGATTCCGTGAAGGGTCGCTTTACCATCT |
| | CTGCAGACACAAGTAAGAATACCGCCTATCTGCAGATGAACTCACT |
| | GAGAGCAGAGGATACAGCTGTTTATTACTGCTCCCGGTGGGGTGGC |
| | GATGGTTTTTATGCTATGGACGTATGGGGCCAGGGCACCCTGGTTA |
| | CCGTCTCTAGCTCCGGTACCACAACCCCTGCTCCACGCCCGCCCACT |
| | CCGGCCCCTACTATTGCCAGCCAACCCTTGAGTCTCAGGCCTGAAG |
| | CCTGTCGCCCCGCGGCCGGAGGCGCAGTGCATACACGCGGCCTGGA |
| | TTTCGCCTGTGACATTTATATCTGGGCACCCTTGGCCGGCACCTGCG |
| | GTGTTCTGCTTCTGAGCTTGGTGATCACTTTGTATTGCCGTGTGAAG |
| | TTCAGCCGCTCTGCTGATGCACCTGCCTATCAACAGGGGCAGAACC |
| | AGCTCTATAACGAGTTGAACCTGGGCCGGCGCGAGGAATACGATGT |
| | CCTCGATAAACGCAGAGGACGTGATCCAGAGATGGGTGGAAAGCC |
| | ACGCCGTAAGAACCCCCAGGAGGGTCTGTACAATGAGCTGCAGAA |
| | GGATAAAATGGCAGAGGCTTACAGCGAAATTGGGATGAAGGGGGA |
| | GCGGCGCCGTGGCAAGGGGCATGACGGCCTTTATCAAGGCCTGTCC |
| | ACCGCCACCAAGGATACTTATGACGCCCTGCACATGCAAGCGCTGC |
| | CCCCGCGGTCCCCCAATCGCAAGAACCCGCTGTGGCCTAGCGTACC |
| | AGATCCCGCGCATTCCAGCCTGGGCTCTTGGGTGCCGACAATCATG |
| | GAAGAGGACGCTTTCCAGCTGCCTGGTCTGGGCACTCCTCCCATTA |
| | CGAAACTGACCGTGCTCGAGGAAGATGAGAAAAAGCCCGTGCCTT |
| | GGGAGTCTCATAACTCCAGCGAGACATGCGGTCTGCCCACCCTCGT |
| | GCAGACCCCCACGAGCTTTGGGTACGATAAACCCCACGTGTTGGTC |
| | GACTTGCTCGTCGATGACAGCGGCTAG (SEQ ID NO: 17) |
| CD8L_4D5(aHer2)_CD8H_ | ATGGCTCTCCCGGTGACGGCTTTGCTGCTCCCCCTGGCCCTCCTGTT |
| CD8TM - FCER1GIC - | GCACGCTGCCAGACCTGACATCCAGATGACCCAGTCCCCGTCCAGC |
| GCSF-STAT1 ICD | CTGAGTGCGTCCGTGGGCGACCGCGTGACAATCACCTGCCGCGCAT |
| | CCCAGGATGTTAATACTGCTGTCGCGTGGTACCAACAGAAGCCCGG |
| | CAAGGCACCAAAGCTCCTGATTTACTCTGCCTCTTTTCTCTACAGCG |
| | GAGTACCCTCCCGCTTTAGCGGATCCCGCTCTGGCACGGACTTCAC |
| | CCTGACCATTAGCTCCCTGCAGCCTGAGGATTTCGCAACCTATTACT |
| | GTCAGCAACACTACACCACTCCTCCCACCTTCGGTCAGGGCACTAA |
| | GGTCGAAATCAAGCGCACCGGTAGCACCAGTGGCTCAGGTAAACCT |
| | GGCAGCGGGGAGGGCTCTGAGGTGCAGCTCGTCGAGTCCGGAGGG |
| | GGCTTGGTGCAGCCCGGTGGCTCCCTGCGCCTGTCCTGCGCCGCGT |
| | CTGGATTTAACATCAAAGACACTTACATCCATTGGGTCAGACAGGC |
| | TCCTGGCAAAGGCCTGGAGTGGGTAGCGCGTATCTACCCTACCAAC |
| | GGCTACACCCGCTATGCGGACTCCGTGAAGGGACGCTTCACTATTT |
| | CCGCTGATACCAGCAAGAACACCGCATATTTGCAAATGAACAGCCT |
| | TAGAGCGGAAGACACTGCAGTCTACTATTGCTCTCGGTGGGGAGGC |
| | GATGGGTTCTACGCCATGGACGTGTGGGGTCAGGGCACCCTGGTCA |
| | CTGTGTCTTCCAGCGGTACCACGACCCCTGCTCCCCGTCCACCCACC |
| | CCAGCCCCTACCATTGCATCACAGCCCCTGTCCCTGCGGCCCGAGG |
| | CATGTCGCCCCGCGGCCGGGGGAGCTGTCCATACTCGTGGGCTGGA |
| | CTTTGCCTGTGATATCTATATCTGGGCTCCCCTCGCAGGTACCTGTG |
| | GGGTCCTGCTCCTGAGCCTGGTGATCACCCTGTACTGCCGCCTGAA |
| | GATTCAGGTGCGTAAAGCGGCTATTACTTCCTACGAGAAGAGCGAC |
| | GGTGTGTATACTGGCCTGTCAACCCGTAACCAAGAAACCTACGAGA |
| | CCCTCAAGCATGAGAAACCACCTCAGAGTCCAAATCGGAAGAACC |
| | CACTCTGGCCGTCCGTTCCTGACCCCGCCCATTCCTCTCTCGGCTCA |
| | TGGGTGCCTACTATCATGGAGGAAGACGCTTTCCAGCTGCCGGGCC |
| | TCGGCACGCCGCCAATCACCAAGCTGACCGTGCTGGAGGAAGATG |
| | AAAAAAAGCCTGTTCCCTGGGAATCCCACAACTCCAGTGAGACCTG |
| | CGGCCTCCCTACTTTGGTGCAGACCCCAACATCCTTTGGGTACGATA |
| | AACCGCACGTCCTCGTGGACCTGCTCGTCGACGATTCCGGCTAA |
| | (SEQ ID NO: 18) |
| CD8L_4D5(aHer2)_CD8H_ | ATGGCTCTGCCTGTGACAGCTCTCCTGCTCCCACTCGCCCTGTTGCT |
| CD8TM - GCSF-STAT1 | GCATGCAGCCCGCCCCGATATCCAGATGACCCAATCCCCGAGCTCC |
| ICD - CD3z | CTGTCAGCCAGCGTGGGCGACCGCGTGACCATCACATGCCGCGCCA |
| | GCCAGGACGTGAACACGGCGGTGGCATGGTACCAACAGAAACCCG |
| | GGAAGGCCCCTAAGCTGCTTATTTACAGTGCGTCTTTCCTGTATTCA |
| | GGCGTGCCATCCCGCTTCTCAGGGTCCAGGTCAGGCACCGACTTCA |
| | CCCTTACCATCTCCAGCCTGCAGCCCGAAGACTTCGCTACCTACTAT |
| | TGCCAACAGCATTACACCACTCCCCCTACCTTCGGCCAGGGGACCA |
| | AGGTCGAGATTAAGCGTACAGGCTCTACGAGCGGGAGCGGCAAGC |
| | CAGGTTCCGGTGAGGGGTCTGAGGTGCAGCTTGTCGAATCCGGGGG |
| | CGGGCTGGTGCAACCAGGGGGTTCCCTGCGGCTCAGTTGCGCTGCC |
| | TCCGGGTTCAATATCAAGGACACGTATATCCATTGGGTGAGACAGG |
| | CTCCCGGCAAAGGCCTCGAGTGGGTGGCTAGGATCTACCCAACCAA |
| | CGGATATACCAGATACGCTGACAGCGTGAAGGGCCGCTTCACCATT |
| | TCTGCAGATACCTCTAAGAACACCGCGTACCTGCAGATGAACAGCC |
| | TGCGGGCGGAGGACACTGCCGTGTACTATTGTAGCAGATGGGGAG |
| | GCGACGGCTTTTACGCCATGGATGTCTGGGGCCAGGGGACCCTGGT |
| | GACAGTTAGTTCCTCTGGCACAACGACTCCAGCACCCAGACCCCCT |
| | ACACCTGCCCCCACAATTGCTAGCCAGCCCCTGTCTCTGCGCCCCG |
| | AGGCCTGCCGTCCCGCCGCGGGCGGGGCCGTCCATACGCGGGGCCT |
| | CGACTTCGCTTGCGATATCTATATCTGGGCTCCTCTGGCCGGCACGT |
| | GTGGGGTGCTGCTCCTGTCCCTGGTCATCACTCTGTATTGTTCTCCT |
| | AATAGGAAGAACCCCCTGTGGCCCTCCGTCCCGGACCCAGCCCACA |
| | GCTCTCTGGGGTCCTGGGTGCCGACCATCATGGAGGAAGACGCATT |
| | CCAGCTCCCCGGTCTGGGTACCCCGCCCATCACCAAGCTGACCGTG |
| | CTGGAGGAAGACGAAAAAAAGCCCGTCCCCTGGGAGTCCCACAAC |
| | TCAAGTGAGACTTGTGGACTGCCTACACTTGTTCAGACCCCCACAT |
| | CCTTCGGCTATGACAAGCCTCATGTCCTGGTTGACCTCCTGGTCGAC |
| | GATAGCGGCCGCGTGAAGTTCTCCAGGTCTGCTGATGCCCCGGCGT |
| | ACCAACAGGGTCAGAACCAGCTGTACAACGAGCTTAATTTGGGGCG |
| | GCGCGAAGAGTATGACGTCCTTGACAAGAGACGCGGCCGCGACCC |
| | CGAGATGGGCGGTAAACCCCGCAGGAAGAACCCCCAGGAGGGACT |
| | CTACAATGAACTGCAGAAGGACAAAATGGCTGAGGCATATAGCGA |
| | AATCGGCATGAAGGGCGAGAGACGGCGTGGCAAGGGTCACGACGG |
| | CCTGTATCAAGGCCTGAGCACCGCGACCAAGGATACATACGATGCG |
| | TTGCACATGCAAGCATTGCCCCCTCGCTAG (SEQ ID NO: 19) |
| CD8L 4D5(aHer2)_CD8H_ | ATGGCGCTGCCTGTCACAGCGCTCTTGCTGCCACTTGCTCTGTTGCT |
| CD8TM - GCSF-STAT1 | CCACGCTGCAAGACCCGACATCCAGATGACTCAGTCCCCGAGCTCT |
| ICD - FCER1GIC | TTGTCCGCATCCGTGGGGGACCGCGTGACTATCACCTGTCGGGCGA |
| | GTCAAGACGTGAATACCGCCGTCGCATGGTACCAACAGAAGCCCG |
| | GAAAGGCCCCAAAGCTTTTGATCTATTCTGCTTCTTTTCTGTATTCC |
| | GGCGTCCCCTCTCGCTTCTCCGGGAGCCGTTCCGGCACCGACTTCAC |
| | CCTGACCATCAGCTCTCTGCAGCCCGAAGACTTCGCTACATATTACT |
| | GTCAACAGCATTATACTACCCCACCCACCTTCGGCCAGGGCACCAA |
| | AGTCGAAATCAAGAGAACCGGCTCCACTTCTGGCTCCGGTAAGCCA |
| | GGCTCTGGGGAAGGCAGCGAGGTGCAGCTGGTCGAATCTGGCGGT |
| | GGGTTGGTACAGCCCGGCGGATCTCTCCGCCTCTCTTGTGCCGCTTC |
| | CGGCTTCAACATCAAGGACACATACATCCACTGGGTGCGGCAGGCT |
| | CCTGGTAAGGGCCTGGAATGGGTCGCCAGGATCTATCCTACAAACG |
| | GCTACACAAGGTACGCCGACTCCGTGAAGGGCCGCTTTACCATCAG |
| | TGCCGATACTTCCAAGAATACCGCCTACCTGCAAATGAACTCACTG |
| | CGCGCTGAGGATACTGCTGTCTACTATTGCAGCCGGTGGGGCGGAG |
| | ACGGTTTCTACGCCATGGATGTCTGGGGACAGGGGACCCTGGTGAC |
| | CGTAAGCTCTTCCGGTACAACCACACCTGCGCCTAGGCCCCCTACG |
| | CCGGCCCCTACTATTGCCAGCCAACCACTTTCTCTGCGCCCGGAGG |
| | CGTGCCGCCCTGCAGCCGGCGGTGCCGTCCACACCCGCGGCCTGGA |
| | TTTTGCCTGCGACATCTATATTTGGGCTCCTCTGGCGGGAACCTGTG |
| | GCGTGCTGCTCTTGTCTTTGGTGATCACCCTGTACTGTTCCCCTAAC |
| | CGTAAGAACCCTCTGTGGCCCAGCGTGCCTGATCCGGCACATTCTT |
| | CCTTGGGCTCTTGGGTGCCGACCATCATGGAGGAAGATGCTTTTCA |
| | GCTGCCTGGACTGGGTACGCCGCCTATCACCAAGCTGACAGTCTTG |
| | GAAGAGGATGAGAAAAAGCCTGTACCATGGGAGTCTCATAATAGC |
| | TCTGAGACCTGTGGCTTGCCTACTCTGGTGCAGACACCTACTTCCTT |
| | CGGCTATGACAAGCCCCATGTCCTGGTGGATCTTCTGGTGGATGAC |
| | AGCGGCAGACTGAAGATCCAGGTGCGTAAGGCAGCCATCACTTCCT |
| | ATGAAAAGTCCGACGGCGTGTATACCGGTCTGTCAACTCGCAATCA |
| | GGAAACCTACGAGACCCTTAAGCACGAGAAGCCTCCCCAATAG |
| | (SEQ ID NO: 20) |
| CD8L_4D5(aHer2)_CD28H_ | ATGGCCCTGCCCGTTACCGCCCTGTTGCTGCCACTGGCTCTCCTGCT |
| CD28TM - CD3z - GCSF- | TCATGCCGCTCGCCCAGATATTCAGATGACTCAGTCTCCTAGCTCTC |
| STAT1 ICD | TGAGCGCTAGCGTGGGAGACCGCGTCACCATCACATGCCGCGCCAG |
| | CCAGGACGTGAACACCGCCGTGGCGTGGTACCAACAGAAGCCCGG |
| | AAAAGCTCCCAAGCTGCTCATTTACTCCGCATCTTTCCTGTATTCCG |
| | GCGTGCCCAGTCGTTTTAGCGGCTCAAGGTCTGGAACCGACTTTAC |
| | ACTGACTATTAGCTCTCTGCAACCAGAGGACTTTGCAACTTACTATT |
| | GCCAACAGCACTACACTACACCACCTACATTTGGCCAGGGCACAAA |
| | GGTTGAGATCAAGCGCACCGGGAGTACATCCGGTTCCGGCAAGCCC |
| | GGGTCTGGCGAGGGCTCCGAGGTACAGCTGGTCGAGAGCGGGGGA |
| | GGCCTGGTCCAGCCGGGTGGCAGCCTGCGTCTCAGCTGCGCCGCAT |
| | CCGGCTTTAACATTAAGGACACCTATATCCATTGGGTGCGTCAGGC |
| | TCCAGGTAAGGGACTTGAGTGGGTGGCGCGGATTTACCCTACGAAC |
| | GGATACACAAGGTACGCTGACAGCGTGAAAGGACGCTTTACCATCA |
| | GCGCCGATACGAGCAAGAATACCGCTTACCTCCAGATGAATTCCCT |
| | GCGCGCCGAGGACACCGCGGTGTATTACTGCTCTCGCTGGGGTGGC |
| | GATGGCTTCTATGCCATGGACGTGTGGGGACAGGGGACACTGGTGA |
| | CCGTAAGCTCTAGTGGAATCGAGGTGATGTATCCGCCTCCCTATCT |
| | GGACAATGAGAAAAGCAACGGCACTATCATTCATGTGAAAGGTAA |
| | GCATCTTTGTCCCAGTCCCCTGTTTCCTGGGCCGTCCAAGCCCTTTT |
| | GGGTGCTGGTGGTTGTCGGCGGAGTGCTGGCGTGTTATTCTCTGTTG |
| | GTCACAGTGGCATTCATTATCTTTTGGGTGCGTGTGAAATTTTCCAG |
| | ATCCGCTGACGCCCCCGCATACCAACAGGGTCAGAACCAGCTCTAC |
| | AACGAGCTTAATCTGGGCCGTAGGGAAGAGTACGATGTGCTTGATA |
| | AGCGTCGCGGTCGTGACCCCGAGATGGGCGGTAAGCCTAGACGCA |
| | AGAACCCCCAGGAGGGGCTGTACAATGAGCTCCAGAAGGACAAGA |
| | TGGCCGAAGCCTACTCTGAGATTGGCATGAAGGGCGAAAGACGCA |
| | GGGGCAAAGGCCATGATGGGCTGTACCAGGGCCTGTCTACCGCTAC |
| | CAAAGATACATACGATGCTCTGCATATGCAGGCCCTGCCTCCCCGC |
| | TCCCCTAACCGCAAAAACCCACTCTGGCCTAGCGTGCCTGATCCCG |
| | CCCACAGCAGTCTCGGTAGCTGGGTGCCCACCATCATGGAAGAGGA |
| | CGCTTTCCAATTGCCTGGGCTGGGCACCCCACCCATTACCAAGTTG |
| | ACCGTACTCGAAGAGGACGAGAAGAAACCCGTCCCTTGGGAGAGT |
| | CACAACTCTTCCGAGACCTGTGGCTTGCCCACCCTGGTGCAGACAC |
| | CTACCTCCTTCGGATACGATAAGCCCCACGTCCTCGTGGATCTCCTG |
| | GTCGACGATAGTGGTTAG (SEQ ID NO: 21) |
| CD8L_4D5(aHer2)_CD28H_ | ATGGCTCTGCCTGTGACCGCCTTGCTCCTGCCGCTGGCACTGCTCCT |
| CD28TM - CD3z - GCSF- | GCACGCTGCACGCCCCGATATCCAGATGACGCAGTCTCCAAGCTCT |
| STAT1 ICD-F | CTGTCAGCCTCCGTGGGAGACCGCGTGACAATTACATGCCGCGCCT |
| | CTCAGGACGTTAATACAGCCGTGGCCTGGTATCAACAGAAACCCGG |
| | AAAGGCACCCAAACTTCTGATCTACTCTGCGTCCTTTCTCTATAGTG |
| | GCGTACCCTCACGTTTCAGCGGGAGTAGAAGCGGAACCGACTTTAC |
| | CCTGACAATTAGTAGCCTGCAGCCCGAGGATTTCGCCACGTACTAT |
| | TGTCAACAGCACTACACTACCCCTCCCACCTTCGGCCAGGGGACTA |
| | AAGTAGAAATTAAGCGCACCGGTTCTACTTCCGGGAGCGGCAAACC |
| | GGGGTCCGGAGAGGGCTCTGAGGTGCAGCTCGTTGAGTCCGGCGGT |
| | GGATTGGTCCAGCCGGGAGGGTCTCTGAGGCTGTCCTGCGCAGCTT |
| | CTGGCTTCAACATTAAGGACACCTACATCCATTGGGTGAGGCAGGC |
| | CCCTGGTAAAGGACTGGAGTGGGTTGCCAGGATCTATCCTACCAAT |
| | GGTTACACCCGTTATGCGGACAGCGTGAAGGGCCGGTTCACCATCT |
| | CTGCGGACACCTCCAAGAACACCGCGTACCTGCAGATGAACTCCCT |
| | CCGCGCCGAGGACACAGCCGTTTACTATTGTAGCCGTTGGGGTGGC |
| | GACGGGTTTTACGCTATGGACGTGTGGGGACAGGGCACCCTGGTTA |
| | CCGTCTCTAGCTCTGGCATCGAGGTGATGTATCCCCCTCCATATCTC |
| | GACAACGAGAAATCCAACGGCACCATTATCCACGTGAAGGGTAAA |
| | CACCTGTGCCCGTCCCCTCTGTTCCCCGGTCCCAGCAAGCCATTCTG |
| | GGTCCTGGTAGTGGTCGGTGGCGTGCTGGCGTGTTACAGCCTTCTG |
| | GTGACCGTGGCGTTTATTATCTTCTGGGTGCGCGTGAAGTTCAGCCG |
| | CTCTGCTGACGCCCCAGCCTACCAACAGGGCCAGAACCAGCTCTAC |
| | AACGAGCTCAACCTGGGCAGGCGCGAAGAGTATGATGTACTGGAC |
| | AAGAGGAGAGGTCGGGACCCGGAGATGGGCGGAAAGCCCCGCAGG |
| | AAGAACCCTCAGGAAGGCTTGTACAACGAGCTCCAAAAAGACAAA |
| | ATGGCTGAGGCCTACAGCGAGATCGGGATGAAAGGGGAACGTCGC |
| | CGGGGGAAAGGCCACGACGGGCTCTATCAGGGGCTGTCCACAGCA |
| | ACCAAGGATACCTACGATGCACTCCACATGCAGGCTCTGCCGCCCA |
| | GAAGCCCAAACCGCAAGAACCCCCTGTGGCCCTCCGTGCCCGACCC |
| | CGCCCACTCAAGCCTCGGCAGCTGGGTGCCTACCATCATGGAGGAA |
| | GACGCCTTCCAGCTCCCTGGTCTGGGCACGCCGCCCATCACCAAGC |
| | TGACCGTTCTGGAGGAAGACGAGAAAAAGCCCGTTCCCTGGGAGTC |
| | TCATAACTCCAGCGAAACCTGTGGTCTCCCTACCCTCGTGCAGACG |
| | CCGACCAGCTTCGGATTCGATAAGCCTCACGTCCTGGTCGATCTTCT |
| | GGTGGATGACTCTGGTTAA (SEQ ID NO: 22) |
| CD8L_4D5(aHer2)_CD28H_ | ATGGCCTTGCCTGTTACAGCGCTGTTGCTGCCACTGGCCCTTCTGCT |
| CD28TM - FCER1GIC - | TCACGCTGCACGCCCTGACATCCAGATGACCCAGTCCCCTAGTTCC |
| GCSF-STAT1 ICD | CTGTCTGCCTCTGTGGGAGACCGGGTCACTATCACATGCAGAGCAA |
| | GTCAAGACGTGAATACCGCCGTGGCCTGGTACCAACAGAAGCCAG |
| | GCAAGGCACCAAAGTTGCTGATCTACAGCGCCTCCTTCCTGTACAG |
| | CGGCGTCCCCTCCCGCTTTTCCGGCTCCCGCTCAGGGACCGATTTCA |
| | CCCTTACCATCAGCTCCCTTCAACCGGAGGATTTTGCCACATATTAC |
| | TGCCAGCAACACTACACCACTCCGCCTACTTTCGGACAAGGGACCA |
| | AGGTAGAAATTAAGAGAACCGGCTCCACTAGTGGCTCCGGTAAACC |
| | AGGCTCAGGTGAGGGATCTGAGGTCCAGCTCGTGGAGAGCGGGGG |
| | TGGACTGGTTCAGCCGGGTGGCTCTCTCCGCCTCAGTTGCGCTGCCT |
| | CCGGCTTTAACATTAAGGATACATACATTCACTGGGTTCGCCAGGC |
| | CCCAGGAAAGGGCCTCGAGTGGGTGGCCAGAATCTACCCAACTAAT |
| | GGCTACACTCGTTATGCTGACTCCGTGAAGGGCAGGTTTACCATCA |
| | GCGCCGACACCTCTAAAAACACCGCCTACCTGCAGATGAACAGTCT |
| | CCGCGCCGAGGACACGGCCGTGTATTACTGCTCACGCTGGGGAGGC |
| | GACGGATTTTACGCTATGGACGTTTGGGGGCAGGGCACTCTGGTGA |
| | CAGTGTCTTCCAGCGGCATCGAAGTCATGTACCCACCGCCTTACCT |
| | GGATAACGAGAAGTCCAACGGCACAATCATTCACGTCAAAGGTAA |
| | ACACCTCTGTCCAAGTCCGTTGTTCCCCGGCCCTAGCAAACCCTTCT |
| | GGGTTCTTGTGGTTGTGGGTGGCGTCCTGGCTTGTTATAGCCTTCTG |
| | GTGACAGTAGCGTTTATTATCTTCTGGGTGCGTCTTAAGATCCAAGT |
| | GCGCAAAGCTGCCATCACCTCCTACGAGAAGTCTGACGGTGTATAC |
| | ACCGGTCTGTCAACTCGCAACCAGGAAACCTATGAGACACTGAAGC |
| | ACGAAAAACCCCCGCAGAGCCCGAACCGCAAGAACCCCCTGTGGC |
| | CTTCAGTGCCCGACCCTGCCCACTCCTCTCTGGGTTCTTGGGTCCCC |
| | ACCATCATGGAAGAGGATGCCTTCCAGCTCCCAGGTCTCGGTACGC |
| | CCCCAATTACAAAGCTGACCGTGCTGGAAGAGGACGAGAAAAAGC |
| | CAGTCCCTTGGGAGAGCCACAATAGCTCTGAGACCTGCGGACTCCC |
| | CACTCTGGTGCAAACCCCAACCTCCTTCGGTTATGATAAACCGCAT |
| | GTGCTTGTGGACCTCCTGGTGGACGATTCTGGATAG (SEQ ID NO: |
| | 23) |
| CD8L_4D5(aHer2)_CD28H_ | ATGGCTCTTCCCGTCACGGCCCTCCTGTTGCCACTTGCTCTCTTGCT |
| CD28TM - FCER1GIC - | GCACGCTGCCCGCCCAGACATCCAGATGACACAGAGCCCCAGCTCT |
| GCSF-STAT1-F | CTCAGCGCTTCTGTAGGGGATCGCGTCACTATCACCTGTCGCGCCA |
| | GTCAGGACGTAAACACTGCGGTCGCTTGGTACCAACAGAAGCCTGG |
| | AAAGGCCCCGAAGCTTCTGATTTACTCCGCTTCCTTCCTGTATTCTG |
| | GCGTGCCTAGCCGTTTCTCCGGGAGTCGCTCAGGCACCGACTTTACT |
| | CTGACCATCAGCTCTCTTCAGCCTGAGGACTTCGCCACCTATTACTG |
| | CCAACAGCATTATACGACTCCCCCTACTTTCGGTCAGGGCACCAAA |
| | GTTGAGATCAAGCGTACCGGCTCTACATCCGGTTCCGGCAAACCCG |
| | GCTCCGGAGAAGGGTCCGAAGTGCAACTGGTAGAGTCTGGGGGCG |
| | GGCTGGTGCAGCCCGGCGGATCCCTGCGCCTGAGTTGTGCAGCCAG |
| | CGGCTTTAATATCAAGGACACTTACATCCACTGGGTGCGCCAAGCA |
| | CCTGGAAAGGGCCTGGAGTGGGTGGCCCGCATCTATCCAACCAACG |
| | GCTACACACGTTATGCTGACTCCGTGAAGGGACGCTTCACAATCAG |
| | CGCCGACACCTCCAAGAACACCGCCTATCTGCAGATGAACTCCCTG |
| | CGCGCTGAGGACACTGCCGTTTACTATTGCTCTCGCTGGGGCGGTG |
| | ACGGATTCTACGCCATGGATGTTTGGGGCCAGGGAACCCTGGTGAC |
| | GGTGTCCTCAAGCGGCATCGAAGTTATGTATCCACCGCCCTACCTT |
| | GACAACGAGAAGAGCAACGGCACTATTATCCACGTGAAGGGCAAA |
| | CACCTGTGCCCTAGCCCCCTTTTCCCCGGTCCCAGTAAGCCCTTCTG |
| | GGTGTTGGTTGTGGTCGGTGGAGTGTTGGCCTGCTACAGTTTGCTGG |
| | TTACTGTTGCCTTCATCATTTTTTGGGTGCGTCTGAAGATTCAGGTT |
| | AGAAAGGCTGCAATCACAAGCTACGAAAAGAGCGACGGCGTGTAT |
| | ACTGGGCTGTCTACCAGGAACCAGGAGACCTATGAAACTCTGAAGC |
| | ACGAGAAGCCCCCGCAGTCTCCAAACAGGAAAAACCCCCTGTGGC |
| | CTTCAGTGCCGGATCCCGCTCACAGCTCACTGGGATCTTGGGTCCCT |
| | ACTATCATGGAAGAGGACGCCTTCCAGCTGCCTGGACTGGGCACTC |
| | CGCCTATTACCAAACTGACCGTGTTGGAGGAAGATGAGAAAAAGC |
| | CGGTACCCTGGGAGAGCCACAATAGTTCCGAAACTTGCGGTCTGCC |
| | TACCCTGGTTCAAACTCCCACGTCTTTTGGTTTTGACAAGCCGCATG |
| | TGCTGGTGGACTTGCTGGTGGATGACTCCGGCTAA (SEQ ID NO: 24) |
| CD8L_4D5(aHer2)_CD28H_ | ATGGCTCTGCCAGTCACCGCACTGCTCCTGCCTCTGGCTCTGCTTCT |
| CD28TM - GCSF-STAT1 | GCACGCTGCAAGACCTGATATCCAGATGACACAGTCCCCTAGCTCC |
| ICD - CD3z | TTGAGTGCCTCTGTGGGCGACCGCGTGACAATCACCTGCCGCGCCT |
| | CCCAGGACGTGAACACCGCCGTTGCTTGGTACCAACAGAAACCAGG |
| | GAAAGCCCCAAAGCTTCTGATTTACTCTGCTAGCTTCCTGTATTCTG |
| | GCGTGCCGTCTCGGTTCTCTGGCAGTCGCTCCGGAACCGACTTCACT |
| | TTGACCATCAGCTCCCTCCAACCAGAGGACTTTGCCACTTATTACTG |
| | TCAGCAACACTACACCACTCCCCCAACCTTCGGTCAGGGCACCAAG |
| | GTCGAGATTAAACGCACGGGTTCCACTTCCGGCTCTGGTAAACCAG |
| | GCAGCGGCGAGGGCTCCGAAGTGCAACTTGTGGAGTCCGGTGGCG |
| | GTCTGGTGCAGCCTGGCGGATCTCTGCGCCTCAGCTGCGCGGCCTC |
| | CGGGTTCAATATCAAGGACACATACATCCACTGGGTGAGGCAAGCC |
| | CCTGGCAAGGGGCTGGAGTGGGTTGCCCGCATCTACCCAACCAATG |
| | GGTACACTCGTTACGCCGACTCCGTCAAGGGTAGATTCACGATCTC |
| | TGCCGATACATCTAAGAACACCGCATACCTCCAAATGAATTCCCTG |
| | CGTGCCGAGGATACCGCTGTCTATTACTGTTCCAGATGGGGTGGCG |
| | ACGGATTTTACGCCATGGATGTCTGGGGCCAGGGCACCTTGGTCAC |
| | TGTGTCCAGCAGTGGTATTGAAGTCATGTACCCGCCACCGTACCTG |
| | GACAATGAGAAGTCAAACGGGACTATTATCCATGTAAAGGGAAAG |
| | CACCTGTGCCCCTCTCCACTCTTCCCTGGTCCCAGTAAACCATTCTG |
| | GGTGCTGGTTGTCGTGGGTGGCGTCCTGGCCTGTTATTCCCTCCTGG |
| | TGACAGTGGCGTTCATTATCTTTTGGGTGAGCCCAAACCGCAAGAA |
| | CCCACTGTGGCCCTCTGTGCCTGATCCTGCGCACTCCAGCCTGGGCT |
| | CCTGGGTGCCTACTATCATGGAAGAGGACGCCTTCCAGCTGCCTGG |
| | GCTCGGAACCCCTCCAATCACGAAGCTGACTGTGCTGGAGGAAGAC |
| | GAGAAAAAGCCTGTGCCATGGGAATCCCATAACTCTTCCGAAACCT |
| | GCGGTCTGCCAACTCTGGTTCAGACACCCACATCTTTCGGTTACGAT |
| | AAGCCTCATGTCCTGGTAGACTTGCTGGTAGATGACAGTGGAAGAG |
| | TGAAGTTTTCCCGCTCAGCCGACGCTCCGGCTTACCAACAGGGTCA |
| | AAACCAGCTCTACAACGAGCTGAACCTCGGCCGCCGTGAAGAGTAT |
| | GACGTGCTCGATAAGCGTCGGGGCCGCGATCCCGAGATGGGAGGC |
| | AAGCCGCGCCGTAAAAATCCGCAGGAGGGTCTTTACAACGAACTGC |
| | AGAAGGACAAGATGGCCGAGGCCTACTCCGAGATCGGCATGAAGG |
| | GCGAGCGTAGGCGTGGCAAGGGCCACGACGGCCTGTACCAGGGCC |
| | TCTCTACGGCTACAAAAGACACCTACGACGCTCTGCACATGCAAGC |
| | TCTGCCTCCACGGTGA (SEQ ID NO: 25) |
| CD8L_4D5(aHer2)_CD28H_ | ATGGCTCTGCCCGTAACTGCACTGCTCCTGCCTCTGGCCCTCCTGCT |
| CD28TM - GCSF-STAT1 | CCACGCGGCCCGCCCCGACATTCAGATGACTCAGAGCCCATCCAGT |
| ICD - FCER1GIC | CTGAGTGCTAGCGTGGGTGACAGGGTCACCATTACCTGTCGTGCCT |
| | CTCAGGACGTGAACACAGCGGTGGCTTGGTATCAACAGAAGCCCG |
| | GGAAGGCTCCAAAGCTGCTCATTTACTCAGCCTCCTTCTTGTATTCT |
| | GGAGTGCCCTCCCGCTTTAGCGGGTCAAGGAGCGGAACAGATTTCA |
| | CTCTGACCATCTCTTCACTGCAGCCGGAGGATTTCGCCACCTATTAC |
| | TGCCAACAGCACTATACCACTCCCCCAACGTTTGGCCAGGGGACCA |
| | AGGTGGAGATTAAGAGAACCGGGTCCACTTCTGGGAGCGGTAAGC |
| | CCGGATCCGGAGAGGGCAGCGAAGTTCAGCTTGTCGAGAGCGGCG |
| | GAGGCCTGGTTCAGCCAGGGGGCTCTCTGCGCCTGTCATGCGCGGC |
| | CTCTGGCTTTAACATCAAGGACACCTATATCCATTGGGTGCGCCAG |
| | GCACCCGGTAAAGGCCTGGAATGGGTGGCGCGCATCTACCCCACTA |
| | ACGGATACACGAGGTATGCCGACTCCGTTAAGGGCCGCTTCACCAT |
| | CAGCGCTGATACCAGCAAGAACACCGCCTACTTGCAGATGAATAGT |
| | CTCCGGGCGGAGGACACGGCAGTCTATTACTGTAGCAGGTGGGGA |
| | GGCGATGGCTTCTATGCCATGGACGTGTGGGGACAGGGCACTCTGG |
| | TGACCGTATCCAGCAGTGGAATCGAGGTGATGTACCCTCCCCCTTA |
| | CCTGGATAACGAGAAAAGTAACGGTACCATTATCCACGTGAAGGGT |
| | AAGCATCTGTGTCCTTCTCCCCTGTTCCCCGGGCCATCCAAGCCCTT |
| | TTGGGTCCTGGTTGTGGTAGGTGGGGTTCTGGCCTGTTACAGTCTCC |
| | TGGTCACTGTGGCCTTCATCATTTTCTGGGTCTCTCCTAACAGGAAG |
| | AATCCGCTGTGGCCAAGCGTTCCTGACCCCGCTCATAGTTCTCTGGG |
| | CTCCTGGGTGCCAACCATCATGGAGGAAGACGCCTTTCAGTTGCCT |
| | GGCCTGGGCACTCCACCGATCACAAAGTTGACGGTGCTCGAAGAGG |
| | ACGAGAAAAAGCCCGTCCCGTGGGAGTCACACAATTCATCCGAAA |
| | CTTGCGGGCTGCCCACCCTGGTGCAGACGCCGACCAGCTTTGGCTA |
| | CGACAAGCCTCACGTGCTTGTCGACCTCTTGGTCGATGACTCCGGA |
| | AGACTGAAAATCCAGGTTCGCAAAGCAGCTATCACCTCATATGAGA |
| | AAAGCGATGGCGTCTATACAGGACTGTCCACCCGTAATCAGGAGAC |
| | GTACGAAACCCTGAAGCACGAGAAGCCACCCCAGTAA (SEQ ID |
| | NO: 26) |
| CD8L_4D5(aHer2)_CD8H_ | ATGGCCCTTCCCGTGACCGCCCTGTTGCTGCCTCTTGCATTGCTCCT |
| CD8TM - CD3z - GCSF- | GCACGCCGCTCGCCCCGACATCCAGATGACCCAGTCTCCCTCTAGC |
| STAT1 ICD - (G4S)*3 | CTGTCCGCTAGCGTGGGAGACCGGGTGACTATCACATGCCGCGCGA |
| Linker - STAT1 ICD | GCCAAGACGTGAATACTGCTGTGGCTTGGTACCAACAGAAGCCAGG |
| | GAAGGCCCCCAAACTCCTGATCTACTCTGCAAGCTTTCTTTACTCCG |
| | GTGTGCCGTCAAGATTCAGCGGCAGCAGAAGCGGCACCGACTTTAC |
| | ACTGACCATTTCTAGCCTCCAACCGGAGGACTTCGCTACCTACTATT |
| | GTCAACAGCACTACACTACGCCCCCTACCTTCGGTCAGGGCACCAA |
| | AGTGGAGATTAAGCGTACTGGTTCAACATCCGGCTCCGGGAAGCCC |
| | GGCAGCGGCGAGGGCTCCGAGGTGCAACTGGTGGAGTCTGGTGGC |
| | GGACTCGTGCAGCCTGGGGGCTCCCTTCGCCTCTCATGCGCAGCGA |
| | GTGGCTTCAACATCAAAGACACTTATATCCACTGGGTGCGTCAGGC |
| | ACCGGGTAAGGGCCTGGAGTGGGTGGCTAGAATCTATCCTACCAAC |
| | GGATACACTCGCTACGCAGATTCCGTTAAAGGCCGGTTCACCATCT |
| | CCGCCGACACTTCCAAGAACACCGCTTATCTCCAGATGAACAGCCT |
| | TCGCGCGGAAGACACCGCTGTGTACTATTGCTCCCGCTGGGGTGGC |
| | GATGGCTTCTATGCCATGGACGTGTGGGGCCAAGGAACCCTGGTGA |
| | CCGTGTCCTCTAGCGGTACCACTACCCCGGCCCCAAGACCACCGAC |
| | CCCTGCTCCAACCATCGCTTCTCAGCCTCTTTCACTGCGGCCGGAAG |
| | CTTGTCGCCCCGCTGCCGGGGGCGCTGTCCATACAAGAGGCCTCGA |
| | TTTTGCCTGTGACATCTACATCTGGGCACCGCTCGCTGGAACCTGTG |
| | GTGTGCTGCTCCTGAGCCTCGTGATCACCCTGTATTGCAGAGTGAA |
| | GTTCAGCCGCAGTGCAGACGCCCCTGCCTACCAACAGGGGCAGAA |
| | CCAACTGTACAACGAGCTGAACCTGGGCCGTAGAGAAGAGTACGA |
| | TGTGCTGGATAAGAGGCGGGGCCGGGATCCTGAAATGGGAGGCAA |
| | GCCCCGGCGCAAGAACCCACAAGAAGGCCTGTACAACGAGCTGCA |
| | GAAGGATAAGATGGCTGAGGCATATAGTGAGATTGGAATGAAAGG |
| | AGAGCGCAGACGCGGCAAGGGCCACGACGGCCTGTACCAGGGACT |
| | GAGCACGGCAACAAAAGACACCTACGACGCTTTGCACATGCAGGC |
| | CCTGCCCCCGCGCAGTCCCAATCGCAAAAATCCCTTGTGGCCCTCC |
| | GTCCCTGATCCGGCTCACAGCTCCCTCGGGTCCTGGGTGCCCACCA |
| | TCATGGAAGAGGATGCTTTCCAGCTCCCAGGCCTGGGCACTCCCCC |
| | AATCACCAAGTTGACCGTCTTGGAAGAGGACGAGAAAAAGCCTGT |
| | GCCCTGGGAGTCCCATAACTCCTCAGAGACATGCGGTCTGCCAACC |
| | CTGGTGCAGACCCCAACATCCTTCGGCTACGATAAACCCCACGTCC |
| | TGGTCGATCTGCTCGTTGACGATTCCGGCGGGGGCGGAGGTAGCGG |
| | TGGCGGTGGGTCCGGCGGGGGCGGGTCACCTACTAGCTTCGGTTAC |
| | GATAAGCCCCATGTCCTGGTGGACTTGCTTGTGGATGACTCAGGCT |
| | AA (SEQ ID NO: 158) |
| CD8L_4D5(aHer2)_CD8H_ | ATGGCGCTGCCTGTCACCGCCCTGCTCCTGCCGTTGGCCCTTCTGCT |
| CD8TM - CD3z - GCSF- | CCACGCAGCCAGACCTGATATTCAAATGACTCAGTCACCTAGCTCC |
| STAT1 ICD - (G4S)*3 | CTCTCCGCTTCAGTGGGTGACCGCGTGACAATCACTTGCCGCGCCA |
| Linker - STAT1 ICD - | GCCAGGACGTGAACACTGCCGTGGCCTGGTACCAACAGAAGCCGG |
| (G4S)*3 Linker - STAT1 | GAAAGGCCCCGAAGCTCCTGATTTATTCTGCATCCTTTCTGTATTCA |
| ICD | GGTGTTCCAAGCAGATTTTCTGGTAGCCGCTCCGGAACAGATTTTA |
| | CACTGACAATCTCCAGCCTGCAGCCCGAAGATTTTGCGACTTATTA |
| | CTGTCAACAGCACTACACCACGCCCCCAACTTTTGGCCAGGGCACT |
| | AAGGTGGAGATCAAGCGTACGGGTTCCACCAGTGGTTCCGGTAAGC |
| | CCGGCAGCGGGGAGGGTAGCGAAGTACAGCTGGTGGAGTCCGGCG |
| | GGGGCCTGGTCCAGCCTGGAGGCTCCCTGAGACTGTCCTGCGCTGC |
| | CAGCGGATTCAACATTAAGGACACATACATCCACTGGGTGCGCCAG |
| | GCACCTGGGAAGGGCCTGGAGTGGGTGGCCCGGATCTACCCTACTA |
| | ACGGATACACCCGCTACGCCGATTCCGTGAAGGGGCGGTTTACTAT |
| | CTCCGCTGATACGTCAAAGAACACCGCTTACCTCCAGATGAACTCT |
| | CTGAGGGCAGAGGATACCGCAGTGTATTACTGCTCTAGGTGGGGTG |
| | GGGACGGGTTCTACGCTATGGATGTATGGGGCCAGGGCACACTGGT |
| | CACTGTATCTAGCTCTGGAACGACAACTCCCGCACCCCGCCCTCCA |
| | ACCCCTGCTCCCACAATCGCCTCTCAGCCACTGAGTTTGAGGCCTG |
| | AGGCCTGTCGCCCAGCCGCTGGAGGTGCTGTCCATACTCGTGGACT |
| | GGACTTTGCCTGTGACATCTATATCTGGGCCCCTCTGGCTGGCACAT |
| | GTGGCGTGTTGCTGCTTTCTCTCGTTATCACCCTGTACTGCCGGGTG |
| | AAATTCAGCCGGTCCGCTGACGCACCAGCTTATCAACAGGGACAAA |
| | ACCAGCTGTACAACGAATTGAATCTGGGGCGGCGTGAGGAATACG |
| | ACGTCCTGGACAAACGTCGCGGGCGCGATCCCGAGATGGGTGGCA |
| | AGCCTCGCAGGAAGAACCCGCAGGAGGGTCTTTACAACGAGCTGC |
| | AGAAGGACAAGATGGCTGAGGCATACAGCGAGATCGGTATGAAGG |
| | GGGAGAGGCGTCGCGGTAAGGGCCATGACGGCTTGTACCAGGGAC |
| | TCTCCACTGCCACAAAAGATACTTACGACGCTCTGCACATGCAGGC |
| | TCTGCCTCCACGTTCCCCCAATAGGAAGAATCCCCTTTGGCCATCCG |
| | TGCCTGACCCTGCCCACAGTTCCCTCGGCTCTTGGGTGCCCACCATC |
| | ATGGAAGAGGATGCCTTTCAGCTGCCCGGTCTGGGCACGCCACCCA |
| | TCACTAAGCTGACCGTGCTGGAAGAGGACGAGAAAAAGCCTGTGC |
| | CTTGGGAAAGCCACAATTCTAGCGAGACCTGTGGCCTCCCGACTTT |
| | GGTGCAGACTCCAACTAGTTTTGGGTACGATAAGCCCCACGTGTTG |
| | GTAGACTTGCTGGTGGATGACTCCGGCGGGGGTGGCGGATCCGGCG |
| | GAGGCGGGAGTGGCGGTGGCGGTTCCCCCACTTCTTTCGGATATGA |
| | CAAGCCGCACGTGCTGGTGGATTTGCTGGTGGATGACTCCGGCGGG |
| | GGCGGAGGGAGCGGTGGCGGAGGCTCCGGGGGCGGGGGCTCTCCC |
| | ACATCTTTTGGCTACGACAAACCTCATGTTCTGGTCGACTTGCTGGT |
| | TGATGACTCCGGATAA (SEQ ID NO: 159) |
| CD8L_4D5(aHer2)_CD8H_ | ATGGCCCTGCCGGTTACCGCGCTGCTTCTGCCCCTGGCACTGTTGCT |
| CD8TM - FCER1GIC - | GCACGCGGCCCGGCCTGACATCCAGATGACACAGTCCCCATCTTCC |
| GCSF-STAT1 ICD - | CTGTCTGCGAGCGTGGGAGACCGTGTGACCATTACCTGCAGGGCTT |
| (G4S)*3 Linker - STAT1 | CCCAGGATGTTAACACTGCTGTGGCTTGGTACCAGCAAAAGCCTGG |
| ICD | TAAGGCTCCCAAGCTCCTGATCTACTCCGCATCTTTCCTCTACTCAG |
| | GCGTGCCTAGCAGGTTTAGTGGTAGCCGGTCTGGAACGGACTTCAC |
| | CCTTACAATTTCTTCCCTGCAGCCAGAAGACTTCGCCACCTATTACT |
| | GTCAACAGCACTATACCACACCTCCCACGTTTGGACAGGGGACCAA |
| | AGTGGAGATCAAGAGAACTGGTTCCACTTCAGGTAGCGGGAAGCC |
| | AGGATCAGGTGAAGGCTCCGAGGTCCAGTTGGTCGAGAGCGGTGG |
| | AGGTCTGGTGCAGCCAGGAGGCTCACTGAGATTGTCTTGTGCTGCC |
| | AGCGGCTTCAATATCAAGGATACCTACATCCACTGGGTACGCCAGG |
| | CCCCCGGCAAGGGACTGGAGTGGGTGGCTCGCATCTACCCTACAAA |
| | CGGCTACACCAGGTACGCGGATAGTGTCAAGGGCCGGTTTACCATT |
| | AGCGCGGACACCAGCAAGAACACCGCTTACCTTCAGATGAACTCCC |
| | TGCGGGCGGAAGACACCGCCGTGTATTACTGTTCTCGCTGGGGCGG |
| | TGACGGATTCTACGCCATGGACGTTTGGGGTCAGGGCACCCTGGTT |
| | ACGGTCTCCAGTTCCGGCACAACCACTCCCGCCCCTAGACCGCCAA |
| | CACCTGCTCCTACGATCGCCTCACAGCCCTTGTCCCTTAGACCAGA |
| | GGCCTGTCGTCCGGCGGCCGGAGGCGCCGTGCACACCAGAGGGCT |
| | CGATTTTGCTTGCGACATCTACATCTGGGCTCCACTGGCGGGAACCT |
| | GCGGGGTGCTTTTGCTGAGCCTGGTTATCACCCTGTACTGTCGCCTG |
| | AAAATCCAGGTGCGCAAAGCTGCCATTACCTCTTACGAAAAGAGTG |
| | ATGGCGTGTATACCGGCCTCTCCACAAGAAACCAGGAAACTTATGA |
| | AACCCTCAAGCACGAGAAGCCCCCACAGTCCCCTAACCGTAAGAA |
| | CCCATTGTGGCCTTCTGTGCCTGATCCCGCTCATAGTAGCCTCGGAT |
| | CCTGGGTCCCGACCATCATGGAAGAGGACGCCTTTCAGCTGCCTGG |
| | CCTCGGGACCCCTCCAATCACAAAGCTGACAGTTCTGGAAGAGGAC |
| | GAGAAAAAGCCAGTGCCCTGGGAATCCCACAATTCTAGCGAGACCT |
| | GTGGCCTGCCCACCCTGGTCCAGACCCCAACTTCTTTCGGATATGAT |
| | AAGCCGCACGTGCTGGTTGATCTTCTGGTAGATGACTCCGGAGGTG |
| | GCGGTGGGTCTGGCGGGGGTGGATCCGGCGGGGGCGGGAGCCCAA |
| | CTTCTTTCGGTTATGATAAGCCTCATGTGCTTGTAGATCTCCTGGTT |
| | GATGACTCCGGCTGA (SEQ ID NO: 160) |
| CD8L_4D5(aHer2)_CD8H_ | ATGGCCCTGCCAGTCACTGCTCTGTTGCTGCCACTGGCCTTGCTCCT |
| CD8TM - FCER1GIC - | GCACGCTGCAAGGCCAGATATTCAGATGACACAGAGCCCATCCTCT |
| GCSF-STAT1 ICD - | CTGTCTGCCAGCGTCGGTGATAGAGTGACCATCACGTGCCGGGCTT |
| (G4S)*3 Linker - STAT1 | CCCAAGACGTCAACACAGCGGTTGCTTGGTATCAACAGAAACCGGG |
| ICD - (G4S)*3 Linker - | CAAGGCACCCAAGCTCCTGATCTATTCCGCCAGTTTCCTGTACTCCG |
| STAT1 ICD | GGGTGCCGTCCCGGTTCAGTGGTTCTCGGTCTGGCACGGATTTTACT |
| | CTGACGATCTCCAGCCTCCAGCCTGAGGACTTCGCCACCTATTACTG |
| | CCAACAGCACTACACAACTCCCCCAACCTTTGGTCAGGGCACCAAG |
| | GTGGAGATCAAGCGTACCGGCTCCACTAGCGGCAGTGGGAAACCT |
| | GGCTCAGGGGAAGGGTCTGAAGTGCAGCTGGTGGAGTCTGGAGGC |
| | GGTCTCGTACAGCCAGGTGGCAGCCTGCGCCTGTCCTGCGCTGCCT |
| | CCGGATTCAACATCAAAGACACCTACATCCATTGGGTTCGCCAAGC |
| | CCCCGGCAAGGGCCTTGAGTGGGTAGCCAGGATTTACCCCACCAAT |
| | GGCTACACCCGCTACGCCGACTCCGTTAAGGGCCGTTTCACGATCT |
| | CTGCAGACACCTCTAAGAATACCGCCTATCTGCAGATGAATTCTCT |
| | GCGCGCTGAGGACACAGCGGTGTACTATTGCAGCAGATGGGGAGG |
| | TGACGGTTTTTATGCCATGGACGTCTGGGGCCAGGGCACTCTGGTT |
| | ACGGTGAGTTCTTCCGGGACTACAACCCCGGCACCCAGGCCCCCTA |
| | CTCCCGCTCCCACCATCGCCTCCCAGCCGCTGTCTCTGCGTCCCGAG |
| | GCCTGTCGTCCGGCGGCAGGGGGCGCCGTGCACACCAGAGGCCTG |
| | GATTTCGCTTGTGACATCTACATCTGGGCTCCACTCGCAGGGACGT |
| | GTGGCGTCCTGCTCCTGAGCCTGGTCATCACCCTCTATTGTCGCCTG |
| | AAGATCCAGGTGCGCAAGGCTGCCATCACCAGCTATGAAAAGTCCG |
| | ACGGAGTATACACAGGACTTTCCACACGCAACCAGGAAACTTACGA |
| | GACTTTGAAGCATGAGAAGCCGCCCCAGAGTCCCAACAGAAAGAA |
| | CCCCCTTTGGCCGAGCGTCCCGGATCCCGCGCATTCTTCCCTGGGAT |
| | CCTGGGTGCCCACCATTATGGAAGAGGATGCATTCCAGCTCCCTGG |
| | CCTCGGCACACCCCCTATTACCAAACTCACCGTGCTGGAAGAGGAT |
| | GAGAAGAAACCTGTGCCATGGGAAAGCCACAACAGCTCCGAGACC |
| | TGTGGCCTGCCTACCCTGGTTCAGACTCCTACATCCTTTGGGTATGA |
| | TAAGCCCCACGTGCTGGTTGATCTTCTGGTTGATGACAGCGGTGGC |
| | GGAGGTGGCAGCGGGGGCGGTGGAAGCGGTGGAGGCGGATCCCCA |
| | ACATCTTTCGGCTACGACAAGCCCCACGTGCTGGTAGACTTGCTGG |
| | TCGATGACTCCGGTGGAGGCGGAGGCTCTGGCGGTGGAGGCTCTGG |
| | AGGCGGTGGCTCCCCTACCAGCTTTGGGTACGATAAACCGCATGTT |
| | CTGGTGGATTTGCTGGTCGATGACTCTGGCTAA (SEQ ID NO: 161) |
| CD8L_4D5(aHer2)_CD28H_ | ATGGCCCTGCCCGTGACCGCCTTGCTCCTGCCCTTGGCCCTTCTCCT |
| CD28TM - CD3z - GCSF- | GCATGCGGCCAGACCCGATATCCAGATGACTCAGTCCCCTTCCAGC |
| STAT1 ICD - (G4S)*3 | CTCTCCGCCTCTGTGGGTGATAGAGTCACCATCACTTGTCGGGCTTC |
| Linker - STAT1 ICD | ACAAGACGTGAACACCGCCGTGGCCTGGTATCAACAGAAGCCCGG |
| | CAAAGCACCAAAACTCCTGATTTACTCCGCCAGCTTCCTGTACAGC |
| | GGAGTGCCCTCCCGCTTCAGCGGGAGTCGGAGCGGCACAGACTTCA |
| | CCCTGACCATTTCCAGTCTGCAGCCAGAGGACTTCGCTACATATTAC |
| | TGTCAACAGCACTACACCACGCCACCTACCTTTGGGCAGGGGACGA |
| | AGGTAGAAATCAAACGCACCGGCTCCACATCCGGAAGCGGAAAGC |
| | CTGGCAGCGGCGAGGGCTCAGAGGTCCAGCTGGTGGAGTCCGGGG |
| | GTGGCCTGGTGCAGCCCGGCGGTTCCCTGCGCCTCTCTTGCGCGGCT |
| | TCCGGTTTCAACATCAAAGACACCTACATCCACTGGGTTCGCCAGG |
| | CCCCAGGAAAGGGCCTCGAGTGGGTGGCTCGCATCTATCCTACCAA |
| | TGGGTACACACGCTACGCCGACTCCGTGAAGGGCCGCTTTACTATC |
| | AGCGCAGACACAAGCAAGAACACAGCCTACCTGCAAATGAACTCA |
| | CTCCGCGCCGAGGACACCGCGGTGTATTACTGCTCCCGCTGGGGAG |
| | GTGACGGCTTCTACGCCATGGACGTGTGGGGGCAGGGAACACTCGT |
| | CACCGTGTCTAGCTCCGGGATCGAAGTAATGTATCCCCCGCCTTAC |
| | CTGGACAACGAGAAATCTAACGGCACGATCATTCATGTCAAGGGA |
| | AAGCACCTGTGCCCATCCCCTCTGTTCCCTGGCCCGTCCAAGCCCTT |
| | TTGGGTTCTGGTGGTTGTCGGAGGCGTGCTGGCATGCTACTCACTCC |
| | TGGTCACCGTTGCCTTTATTATCTTCTGGGTGCGCGTGAAGTTCTCC |
| | AGATCCGCTGATGCCCCAGCATATCAACAGGGACAGAATCAGCTGT |
| | ATAATGAACTGAACCTGGGCAGGCGCGAAGAGTACGATGTGCTCG |
| | ATAAACGCAGGGGCCGCGATCCAGAAATGGGAGGCAAGCCCAGAC |
| | GCAAAAACCCGCAGGAAGGCCTGTATAACGAACTCCAGAAGGACA |
| | AGATGGCCGAGGCCTATAGCGAAATCGGGATGAAGGGCGAACGTA |
| | GACGCGGCAAGGGCCACGACGGGCTCTACCAAGGTCTGTCCACAG |
| | CGACCAAAGATACATACGACGCCCTTCACATGCAAGCCCTGCCCCC |
| | TCGTTCCCCTAACCGTAAGAACCCATTGTGGCCCTCCGTGCCGGAC |
| | CCAGCTCACAGCTCCCTGGGCTCATGGGTGCCCACCATCATGGAAG |
| | AGGACGCATTCCAGCTGCCCGGGCTCGGCACCCCTCCCATCACTAA |
| | GCTTACAGTCCTCGAAGAGGATGAGAAAAAGCCCGTGCCCTGGGA |
| | ATCCCACAACAGCTCCGAGACCTGTGGTCTCCCCACCCTTGTGCAG |
| | ACACCTACTTCCTTCGGGTATGATAAGCCACATGTTCTTGTGGACCT |
| | CTTGGTTGACGATAGCGGGGGTGGGGGCGGTTCCGGCGGGGGTGG |
| | ATCCGGAGGTGGCGGATCCCCCACAAGCTTCGGGTACGACAAGCCC |
| | CACGTTTTGGTGGACCTTCTGGTGGATGACTCCGGTTAA (SEQ ID |
| | NO: 162) |
| CD8L_4D5(aHer2)_CD28H_ | ATGGCGCTGCCCGTGACCGCGCTCCTTTTGCCTCTTGCACTGCTCCT |
| CD28TM - CD3z - GCSF- | GCACGCTGCACGTCCGGACATCCAGATGACTCAATCTCCTAGCAGT |
| STAT1 ICD - (G4S)*3 | TTGTCTGCCTCTGTGGGCGACCGGGTTACCATCACATGTCGGGCCA |
| Linker - STAT1 ICD - | GCCAGGACGTGAACACCGCCGTGGCATGGTATCAACAGAAGCCCG |
| (G4S)*3 Linker - STAT1 | GCAAGGCTCCAAAGCTCCTGATTTACAGTGCCTCTTTCCTGTATTCC |
| ICD | GGTGTACCCTCTCGTTTCTCCGGATCTCGCAGCGGGACGGATTTTAC |
| | CCTGACCATCAGCTCCCTGCAGCCGGAAGATTTTGCTACATACTATT |
| | GCCAACAGCACTACACCACTCCCCCGACTTTCGGCCAGGGCACAAA |
| | GGTGGAGATTAAACGTACCGGCTCAACCTCTGGGTCTGGCAAACCC |
| | GGGTCTGGCGAGGGATCAGAGGTGCAGCTGGTAGAATCAGGCGGT |
| | GGCCTGGTGCAGCCTGGCGGGAGCCTCCGTCTGTCCTGTGCGGCCA |
| | GCGGCTTTAATATCAAGGACACCTATATCCATTGGGTCAGACAAGC |
| | ACCTGGCAAGGGGCTGGAATGGGTTGCTCGTATCTACCCAACTAAT |
| | GGTTATACAAGGTATGCCGACTCTGTCAAGGGCCGTTTTACAATCT |
| | CCGCCGACACCTCTAAAAATACCGCTTATCTCCAGATGAACAGTCT |
| | CAGAGCTGAGGACACAGCCGTGTATTACTGCAGTAGATGGGGCGG |
| | GGATGGCTTCTATGCGATGGATGTCTGGGGGCAGGGCACACTTGTT |
| | ACCGTCTCATCTTCCGGCATCGAGGTGATGTATCCCCCACCTTACCT |
| | TGATAATGAGAAATCAAACGGGACCATTATCCATGTGAAAGGTAA |
| | GCATCTGTGCCCCAGTCCCCTCTTCCCTGGACCTAGCAAGCCCTTCT |
| | GGGTGCTGGTAGTCGTTGGGGGCGTGCTGGCCTGCTACTCCCTGTT |
| | GGTGACCGTGGCCTTTATTATCTTCTGGGTGCGTGTCAAGTTTTCCA |
| | GGTCAGCCGACGCCCCCGCCTACCAGCAAGGTCAGAACCAACTTTA |
| | TAATGAGTTGAATCTGGGACGCCGTGAAGAGTATGACGTCCTCGAC |
| | AAGCGGCGCGGGCGCGATCCCGAGATGGGTGGCAAGCCTCGTCGC |
| | AAGAACCCTCAAGAGGGCTTGTATAACGAGCTGCAGAAGGACAAG |
| | ATGGCTGAAGCTTACTCCGAGATCGGAATGAAGGGCGAGCGTCGCC |
| | GTGGAAAGGGACACGACGGTCTGTACCAAGGTCTCTCCACCGCCAC |
| | TAAGGACACCTACGACGCTCTGCACATGCAAGCCTTGCCGCCCCGC |
| | TCACCCAACCGCAAAAACCCTCTCTGGCCCAGCGTGCCAGATCCTG |
| | CACACTCTTCCCTGGGTAGCTGGGTGCCTACAATTATGGAAGAGGA |
| | CGCCTTTCAGCTCCCGGGTCTGGGAACTCCTCCCATCACCAAGCTG |
| | ACCGTGCTCGAAGAGGACGAGAAGAAACCGGTACCCTGGGAGTCT |
| | CACAACTCTTCCGAGACCTGTGGCCTGCCTACCCTCGTCCAAACTCC |
| | CACTTCATTTGGTTATGACAAGCCTCATGTACTGGTGGACTTGCTGG |
| | TGGACGATTCAGGTGGCGGGGGTGGCTCAGGCGGTGGAGGCTCCG |
| | GGGGCGGGGGCTCTCCTACGTCTTTTGGCTACGACAAGCCTCACGT |
| | TTTGGTCGATTTGCTCGTTGATGACTCTGGCGGAGGCGGTGGCTCCG |
| | GCGGAGGTGGAAGCGGCGGGGGAGGGAGCCCCACTTCCTTCGGCT |
| | ACGATAAGCCTCACGTTCTCGTCGATCTTCTGGTGGACGATTCAGG |
| | ATAA (SEQ ID NO: 163) |
| CD8L_4D5(aHer2)_CD28H_ | ATGGCTCTGCCAGTCACTGCTCTGTTGCTGCCGCTGGCCCTGCTCCT |
| CD28TM - FCER1GIC - | GCACGCCGCTCGCCCTGACATCCAGATGACCCAGTCACCATCCTCA |
| GCSF-STAT1 ICD - | CTGAGCGCCTCTGTCGGAGATAGGGTGACCATTACTTGCCGCGCAT |
| (G4S)*3 Linker - STAT1 | CTCAGGACGTGAACACAGCTGTCGCCTGGTACCAACAGAAGCCCGG |
| ICD | AAAGGCCCCCAAGCTTCTGATTTACTCTGCGAGCTTCCTGTATAGCG |
| | GTGTACCCTCTAGATTCAGTGGAAGCCGGTCAGGTACAGACTTCAC |
| | CCTGACCATTTCATCCCTCCAGCCGGAAGATTTCGCAACATATTACT |
| | GCCAACAGCACTACACTACCCCGCCAACGTTTGGTCAGGGCACAAA |
| | GGTCGAGATCAAACGGACAGGATCCACAAGTGGTTCTGGCAAGCCT |
| | GGCAGTGGTGAGGGCAGCGAAGTTCAATTGGTGGAATCTGGCGGA |
| | GGCCTGGTCCAGCCCGGGGGTAGCCTGCGCCTGTCATGCGCCGCGT |
| | CCGGCTTCAACATTAAGGACACCTACATTCATTGGGTGCGCCAGGC |
| | TCCCGGGAAGGGTCTGGAGTGGGTCGCCCGCATCTACCCCACTAAT |
| | GGCTACACCCGTTACGCTGACTCCGTGAAGGGCCGTTTCACCATTTC |
| | CGCCGACACCTCCAAGAATACCGCATATCTCCAGATGAACTCCCTG |
| | CGCGCTGAGGACACAGCAGTGTATTACTGTTCTCGCTGGGGTGGAG |
| | ACGGTTTCTATGCCATGGACGTTTGGGGCCAGGGCACACTGGTTAC |
| | CGTGAGCTCCAGCGGCATCGAAGTAATGTACCCTCCGCCTTATCTG |
| | GATAACGAGAAATCCAATGGAACCATTATCCACGTCAAAGGCAAA |
| | CACCTGTGTCCGTCCCCTTTGTTCCCTGGACCCTCTAAGCCCTTCTG |
| | GGTGCTCGTCGTGGTAGGCGGGGTGCTGGCCTGTTACTCCCTCCTG |
| | GTAACAGTAGCTTTCATTATCTTCTGGGTGCGCCTGAAGATTCAGGT |
| | CCGTAAGGCAGCTATCACCTCCTATGAGAAGAGCGACGGTGTCTAT |
| | ACCGGCCTGAGCACCCGGAACCAGGAGACTTATGAGACCCTCAAG |
| | CACGAAAAGCCTCCACAGAGTCCCAATCGGAAGAATCCCCTTTGGC |
| | CTTCTGTGCCTGATCCAGCCCACTCCTCTCTTGGTAGCTGGGTGCCT |
| | ACAATTATGGAAGAGGACGCGTTTCAGCTGCCCGGCCTGGGCACCC |
| | CACCCATCACAAAGCTGACCGTGCTCGAGGAAGACGAGAAGAAAC |
| | CCGTGCCATGGGAGAGTCATAACTCAAGCGAGACCTGCGGGTTGCC |
| | CACCCTGGTCCAGACTCCCACCAGCTTTGGATATGACAAGCCTCAT |
| | GTACTGGTGGATTTGCTGGTGGACGATTCCGGGGGCGGAGGCGGTT |
| | CAGGCGGAGGTGGATCCGGTGGAGGTGGCTCTCCCACCTCATTCGG |
| | TTATGACAAGCCCCACGTGTTGGTGGACCTCCTTGTGGACGATTCTG |
| | GTTAA (SEQ ID NO: 164) |
| CD8L_4D5(aHer2)_CD28H_ | ATGGCCCTGCCGGTCACTGCACTCCTTCTGCCGCTGGCACTGCTCCT |
| CD28TM - FCER1GIC - | GCACGCTGCACGTCCCGACATCCAGATGACCCAGTCACCGAGCTCT |
| GCSF-STAT1 ICD - | CTCTCCGCTTCTGTGGGTGACCGTGTGACCATCACCTGCCGTGCCTC |
| (G4S)*3 Linker - STAT1 | TCAGGACGTTAACACCGCTGTGGCCTGGTACCAGCAAAAACCAGGC |
| ICD - (G4S)*3 Linker - | AAAGCCCCCAAACTCCTTATTTACAGTGCTTCATTCCTGTACTCCGG |
| STAT1 ICD | CGTCCCTAGCAGATTTAGCGGCTCCCGCAGCGGAACAGATTTTACA |
| | CTCACCATCTCCTCATTGCAGCCCGAGGATTTTGCCACCTATTACTG |
| | CCAACAGCATTACACAACCCCTCCCACCTTCGGGCAGGGAACTAAG |
| | GTCGAGATCAAGCGCACAGGCTCTACTTCCGGGTCCGGGAAGCCAG |
| | GTTCCGGAGAGGGCTCTGAGGTACAACTGGTTGAAAGTGGGGGCG |
| | GTCTCGTTCAGCCGGGAGGCTCCCTGCGCCTGTCCTGCGCCGCGTCT |
| | GGTTTTAACATTAAAGATACCTATATTCACTGGGTGCGTCAAGCTCC |
| | AGGCAAGGGACTGGAGTGGGTGGCGCGCATCTACCCCACTAACGG |
| | TTACACCCGCTACGCCGACAGCGTCAAGGGGCGTTTTACCATCTCT |
| | GCCGACACCTCCAAGAACACAGCCTATCTTCAAATGAACAGCTTGC |
| | GCGCCGAGGACACCGCGGTGTATTACTGCTCTCGCTGGGGCGGAGA |
| | CGGTTTCTACGCCATGGACGTGTGGGGCCAGGGCACGCTGGTGACT |
| | GTCAGCTCTTCAGGTATCGAGGTCATGTACCCACCCCCATATTTGGA |
| | CAATGAGAAGTCTAATGGCACCATTATCCACGTGAAAGGTAAGCAT |
| | CTCTGTCCCAGTCCGCTGTTTCCCGGTCCAAGTAAGCCTTTTTGGGT |
| | CCTCGTCGTGGTCGGGGGAGTCTTGGCCTGCTACTCTTTGCTGGTGA |
| | CCGTCGCTTTTATTATCTTCTGGGTGCGCCTCAAAATCCAGGTTCGG |
| | AAGGCGGCCATCACAAGCTACGAGAAGAGCGACGGCGTTTATACC |
| | GGCCTGTCCACCCGTAATCAGGAAACATATGAAACCCTGAAGCACG |
| | AAAAGCCACCCCAGAGTCCCAACCGTAAGAACCCACTCTGGCCTTC |
| | TGTCCCCGATCCCGCCCATTCATCCTTGGGTAGTTGGGTGCCAACTA |
| | TCATGGAAGAGGATGCATTCCAGCTCCCTGGCCTGGGCACTCCTCC |
| | CATCACCAAACTGACGGTTCTGGAGGAAGACGAAAAGAAACCGGT |
| | GCCGTGGGAGAGCCACAATTCCTCAGAGACATGTGGTCTGCCAACA |
| | CTGGTGCAAACGCCTACCAGCTTCGGGTATGACAAGCCGCACGTCC |
| | TCGTGGACCTCCTGGTGGATGACTCCGGCGGTGGGGGGGGTCTGG |
| | CGGTGGGGGCTCCGGCGGAGGCGGGTCTCCAACTTCCTTTGGCTAC |
| | GATAAACCACACGTCCTTGTGGACCTTCTGGTGGATGACAGCGGGG |
| | GCGGGGGCGGTAGCGGGGGCGGTGGATCAGGCGGAGGCGGTAGCC |
| | CTACCTCTTTCGGTTATGACAAGCCCCATGTATTGGTAGACCTTCTG |
| | GTGGATGACAGCGGATAG (SEQ ID NO: 165) |

TABLE 4
Exemplary Amino Acid Sequences for Cytokine Receptors

| Intracellular | | | |
|---|---|---|---|
| Signaling | Intracellular | | |
| Output | Domain From: | GeneID | Amino Acid Sequence |
| IL-10 | IL-10Ra | 3587 | MLPCLVVLLAALLSLRLGSDAHGTELPSPPSVWFEAEFF |
| | | | HHILHWTPIPNQSESTCYEVALLRYGIESWNSISNCSQTL |
| | | | SYDLTAVTLDLYHSNGYRARVRAVDGSRHSNWTVTNT |
| | | | RFSVDEVTLTVGSVNLEIHNGFILGKIQLPRPKMAPAND |
| | | | TYESIFSHFREYEIAIRKVPGNFTFTHKKVKHENFSLLTS |
| | | | GEVGEFCVQVKPSVASRSNKGMWSKEECISLTRQYFTV |
| | | | TNVIIFFAFVLLLSGALAYCLALQLYVRRRKKLPSVLLF |
| | | | KKPSPFIFISQRPSPETQDTIHPLDEEAFLKVSPELKNLDL |
| | | | HGSTDSGFGSTKPSLQTEEPQFLLPDPHPQADRTLGNRE |
| | | | PPVLGDSCSSGSSNSTDSGICLQEPSLSPSTGPTWEQQVG |
| | | | SNSRGQDDSGIDLVQNSEGRAGDTQGGSALGHHSPPEP |
| | | | EVPGEEDPAAVAFQGYLRQTRCAEEKATKTGCLEEESP |
| | | | LTDGLGPKFGRCLVDEAGLHPPALAKGYLKQDPLEMT |
| | | | LASSGAPTGQWNQPTEEWSLLALSSCSDLGISDWSFAH |
| | | | DLAPLGCVAAPGGLLGSFNSDLVTLPLISSLQSSE (SEQ |
| | | | ID NO: 27) |
| IL-4/IL-13 | IL4Ra | 3566 | MGWLCSGLLFPVSCLVLLQVASSGNMKVLQEPTCVSD |
| | | | YMSISTCEWKMNGPTNCSTELRLLYQLVFLLSEAHTCIP |
| | | | ENNGGAGCVCHLLMDDVVSADNYTLDLWAGQQLLW |
| | | | KGSFKPSEHVKPRAPGNLTVHTNVSDTLLLTWSNPYPP |
| | | | DNYLYNHLTYAVNIWSENDPADFRIYNVTYLEPSLRIA |
| | | | ASTLKSGISYRARVRAWAQCYNTTWSEWSPSTKWHNS |
| | | | YREPFEQHLLLGVSVSCIVILAVCLLCYVSITKIKKEWW |
| | | | DQIPNPARSRLVAIIIQDAQGSQWEKRSRGQEPAKCPH |
| | | | WKNCLTKLLPCFLEHNMKRDEDPHKAAKEMPFQGSGK |
| | | | SAWCPVEISKTVLWPESISVVRCVELFEAPVECEEEEEV |
| | | | EEEKGSFCASPESSRDDFQEGREGIVARLTESLFLDLLGE |
| | | | ENGGFCQQDMGESCLLPPSGSTSAHMPWDEFPSAGPKE |
| | | | APPWGKEQPLHLEPSPPASPTQSPDNLTCTETPLVIAGN |
| | | | PAYRSFSNSLSQSPCPRELGPDPLLARHLEEVEPEMPCV |
| | | | PQLSEPTTVPQPEPETWEQILRRNVLQHGAAAAPVSAPT |
| | | | SGYQEFVHAVEQGGTQASAVVGLGPPGEAGYKAFSSL |
| | | | LASSAVSPEKCGFGASSGEEGYKPFQDLIPGCPGDPAPV |
| | | | PVPLFTFGLDREPPRSPQSSHLPSSSPEHLGLEPGEKVED |
| | | | MPKPPLPQEQATDPLVDSLGSGIVYSALTCHLCGHLKQ |
| | | | CHGQEDGGQTPVMASPCCGCCCGDRSSPPTTPLRAPDP |
| | | | SPGGVPLEASLCPASLAPSGISEKSKSSSSFHPAPGNAQS |
| | | | SSQTPKIVNFVSVGPTYMRVS (SEQ ID NO: 28) |
| IL7/TSLP | IL7Ra | 3575 | MTILGTTFGMVFSLLQVVSGESGYAQNGDLEDAELDD |
| | | | YSFSCYSQLEVNGSQHSLTCAFEDPDVNITNLEFEICGA |
| | | | LVEVKCLNFRKLQEIYFIETKKFLLIGKSNICVKVGEKSL |
| | | | TCKKIDLTTIVKPEAPFDLSVVYREGANDFVVTFNTSHL |
| | | | QKKYVKVLMHDVAYRQEKDENKWTHVNLSSTKLTLL |
| | | | QRKLQPAAMYEIKVRSIPDHYFKGFWSEWSPSYYFRTP |
| | | | EINNSSGEMDPILLTISILSFFSVALLVILACVLWKKRIKP |
| | | | IVWPSLPDHKKTLEHLCKKPRKNLNVSFNPESFLDCQIH |
| | | | RVDDIQARDEVEGFLQDTFPQQLEESEKQRLGGDVQSP |
| | | | NCPSEDVVITPESFGRDSSLTCLAGNVSACDAPILSSSRS |
| | | | LDCRESGKNGPHVYQDLLLSLGTTNSTLPPPFSLQSGIL |
| | | | TLNPVAQGQPILTSLGSNQEEAYVTMSSFYQNQ (SEQ |
| | | | ID NO: 29) |
| IL9 | IL9Ra | 3581 | MGLGRCIWEGWTLESEALRRDMGTWLLACICICTCVC |
| | | | LGVSVTGEGQGPRSRTFTCLTNNILRIDCHWSAPELGQG |
| | | | SSPWLLFTSNQAPGGTHKCILRGSECTVVLPPEAVLVPS |
| | | | DNFTITFHHCMSGREQVSLVDPEYLPRRHVKLDPPSDL |
| | | | QSNISSGHCILTWSISPALEPMTTLLSYELAFKKQEEAW |
| | | | EQAQHRDHIVGVTWLILEAFELDPGFIHEARLRVQMAT |
| | | | LEDDVVEEERYTGQWSEWSQPVCFQAPQRQGPLIPPW |
| | | | GWPGNTLVAVSIFLLLTGPTYLLFKLSPRVKRIFYQNVP |
| | | | SPAMFFQPLYSVHNGNFQTWMGAHGAGVLLSQDCAG |
| | | | TPQGALEPCVQEATALLTCGPARPWKSVALEEEQEGPG |
| | | | TRLPGNLSSEDVLPAGCTEWRVQTLAYLPQEDWAPTSL |
| | | | TRPAPPDSEGSRSSSSSSSSNNNNYCALGCYGGWHLSA |
| | | | LPGNTQSSGPIPALACGLSCDHQGLETQQGVAWVLAG |
| | | | HCQRPGLHEDLQGMLLPSVLSKARSWTF (SEQ ID NO: |
| | | | 30) |
| IL21 | IL21Ra | 50615 | MPRGWAAPLLLLLLQGGWGCPDLVCYTDYLQTVICILE |
| | | | MWNLHPSTLTLTWQDQYEELKDEATSCSLHRSAHNAT |
| | | | HATYTCHMDVFHFMADDIFSVNITDQSGNYSQECGSFL |
| | | | LAESIKPAPPFNVTVTFSGQYNISWRSDYEDPAFYMLKG |
| | | | KLQYELQYRNRGDPWAVSPRRKLISVDSRSVSLLPLEF |
| | | | RKDSSYELQVRAGPMPGSSYQGTWSEWSDPVIFQTQSE |
| | | | ELKEGWNPHLLLLLLLVIVFIPAFWSLKTHPLWRLWKKI |
| | | | WAVPSPERFFMPLYKGCSGDFKKWVGAPFTGSSLELGP |
| | | | WSPEVPSTLEVYSCHPPRSPAKRLQLTELQEPAELVESD |
| | | | GVPKPSFWPTAQNSGGSAYSEERDRPYGLVSIDTVTVL |
| | | | DAEGPCTWPCSCEDDGYPALDLDAGLEPSPGLEDPLLD |
| | | | AGTTVLSCGCVSAGSPGLGGPLGSLLDRLKPPLADGED |
| | | | WAGGLPWGGRSPGGVSESEAGSPLAGLDMDTFDSGFV |
| | | | GSDCSSPVECDFTSPGDEGPPRSYLRQWVVIPPPLSSPGP |
| | | | QAS (SEQ ID NO: 31) |
| IL2/IL15 | IL2Rb | 3560 | MAAPALSWRLPLLILLLPLATSWASAAVNGTSQFTCFY |
| | | | NSRANISCVWSQDGALQDTSCQVHAWPDRRRWNQTC |
| | | | ELLPVSQASWACNLILGAPDSQKLTTVDIVTLRVLCREG |
| | | | VRWRVMAIQDFKPFENLRLMAPISLQVVHVETHRCNIS |
| | | | WEISQASHYFERHLEFEARTLSPGHTWEEAPLLTLKQK |
| | | | QEWICLETLTPDTQYEFQVRVKPLQGEFTTWSPWSQPL |
| | | | AFRTKPAALGKDTIPWLGHLLVGLSGAFGFIILVYLLIN |
| | | | CRNTGPWLKKVLKCNTPDPSKFFSQLSSEHGGDVQKW |
| | | | LSSPFPSSSFSPGGLAPEISPLEVLERDKVTQLLLQQDKV |
| | | | PEPASLSSNHSLTSCFTNQGYFFFHLPDALEIEACQVYFT |
| | | | YDPYSEEDPDEGVAGAPTGSSPQPLQPLSGEDDAYCTFP |
| | | | SRDDLLLFSPSLLGGPSPPSTAPGGSGAGEERMPPSLQE |
| | | | RVPRDWDPQPLGPPTPGVPDLVDFQPPPELVLREAGEE |
| | | | VPDAGPREGVSFPWSRPPGQGEFRALNARLPLNTDAYL |
| | | | SLQELQGQDPTHLV (SEQ ID NO: 32) |
| IL6/IL11 | gp130 | 3572 | MLTLQTWLVQALFIFLTTESTGELLDPCGYISPESPVVQ |
| | | | LHSNFTAVCVLKEKCMDYFHVNANYIVWKTNHFTIPK |
| | | | EQYTIINRTASSVTFTDIASLNIQLTCNILTFGQLEQNVY |
| | | | GITIISGLPPEKPKNLSCIVNEGKKMRCEWDGGRETHLE |
| | | | TNFTLKSEWATHKFADCKAKRDTPTSCTVDYSTVYFV |
| | | | NIEVWVEAENALGKVTSDHINFDPVYKVKPNPPHNLSV |
| | | | INSEELSSILKLTWTNPSIKSVIILKYNIQYRTKDASTWSQ |
| | | | IPPEDTASTRSSFTVQDLKPFTEYVFRIRCMKEDGKGYW |
| | | | SDWSEEASGITYEDRPSKAPSFWYKIDPSHTQGYRTVQ |
| | | | LVWKTLPPFEANGKILDYEVTLTRWKSHLQNYTVNAT |
| | | | KLTVNLTNDRYLATLTVRNLVGKSDAAVLTIPACDFQA |
| | | | THPVMDLKAFPKDNMLWVEWTTPRESVKKYILEWCV |
| | | | LSDKAPCITDWQQEDGTVHRTYLRGNLAESKCYLITVT |
| | | | PVYADGPGSPESIKAYLKQAPPSKGPTVRTKKVGKNEA |
| | | | VLEWDQLPVDVQNGFIRNYTIFYRTIIGNETAVNVDSSH |
| | | | TEYTLSSLTSDTLYMVRMAAYTDEGGKDGPEFTFTTPK |
| | | | FAQGEIEAIVVPVCLAFLLTTLLGVLFCFNKRDLIKKHI |
| | | | WPNVPDPSKSHIAQWSPHTPPRHNFNSKDQMYSDGNFT |
| | | | DVSVVEIEANDKKPFPEDLKSLDLFKKEKINTEGHSSGI |
| | | | GGSSCMSSSRPSISSSDENESSQNTSSTVQYSTVVHSGY |
| | | | RHQVPSVQVFSRSESTQPLLDSEERPEDLQLVDHVDGG |
| | | | DGILPRQQYFKQNCSQHESSPDISHFERSKQVSSVNEED |
| | | | FVRLKQQISDHISQSCGSGQMKMFQEVSAADAFGPGTE |
| | | | GQVERFETVGMEAATDEGMPKSYLPQTVRQGGYMPQ |
| | | | (SEQ ID NO: 33) |
| IL6 | IL6Ra | 3570 | MLAVGCALLAALLAAPGAALAPRRCPAQEVARGVLTS |
| | | | LPGDSVTLTCPGVEPEDNATVHWVLRKPAAGSHPSRW |
| | | | AGMGRRLLLRSVQLHDSGNYSCYRAGRPAGTVHLLVD |
| | | | VPPEEPQLSCFRKSPLSNVVCEWGPRSTPSLTTKAVLLV |
| | | | RKFQNSPAEDFQEPCQYSQESQKFSCQLAVPEGDSSFYI |
| | | | VSMCVASSVGSKFSKTQTFQGCGILQPDPPANITVTAVA |
| | | | RNPRWLSVTWQDPHSWNSSFYRLRFELRYRAERSKTFT |
| | | | TWMVKDLQHHCVIHDAWSGLRHVVQLRAQEEFGQGE |
| | | | WSEWSPEAMGTPWTESRSPPAENEVSTPMQALTTNKD |
| | | | DDNILFRDSANATSLPVQDSSSVPLPTFLVAGGSLAFGT |
| | | | LLCIAIVLRFKKTWKLRALKEGKTSMHPPYSLGQLVPE |
| | | | RPRPTPVLVPLISPPVSPSSLGSDNTSSHNRPDARDPRSP |
| | | | YDISNTDYFFPR (SEQ ID NO: 34) |
| IL11 | IL11Ra | 3590 | MSSSCSGLSRVLVAVATALVSASSPCPQAWGPPGVQYG |
| | | | QPGRSVKLCCPGVTAGDPVSWFRDGEPKLLQGPDSGL |
| | | | GHELVLAQADSTDEGTYICQTLDGALGGTVTLQLGYPP |
| | | | ARPVVSCQAADYENFSCTWSPSQISGLPTRYLTSYRKK |
| | | | TVLGADSQRRSPSTGPWPCPQDPLGAARCVVHGAEFW |
| | | | SQYRINVTEVNPLGASTRLLDVSLQSILRPDPPQGLRVE |
| | | | SVPGYPRRLRASWTYPASWPCQPHFLLKFRLQYRPAQH |
| | | | PAWSTVEPAGLEEVITDAVAGLPHAVRVSARDFLDAGT |
| | | | WSTWSPEAWGTPSTGTIPKEIPAWGQLHTQPEVEPQVD |
| | | | SPAPPRPSLQPHPRLLDHRDSVEQVAVLASLGILSFLGL |
| | | | VAGALALGLWLRLRRGGKDGSPKPGFLASVIPVDRRPG |
| | | | APNL (SEQ ID NO: 35) |
| GCSF | GCSFR | 1441 | MARLGNCSLTWAALIILLLPGSLEECGHISVSAPIVHLG |
| | | | DPITASCIIKQNCSHLDPEPQILWRLGAELQPGGRQQRLS |
| | | | DGTQESIITLPHLNHTQAFLSCCLNWGNSLQILDQVELR |
| | | | AGYPPAIPHNLSCLMNLTTSSLICQWEPGPETHLPTSFTL |
| | | | KSFKSRGNCQTQGDSILDCVPKDGQSHCCIPRKHLLLY |
| | | | QNMGIWVQAENALGTSMSPQLCLDPMDVVKLEPPMLR |
| | | | TMDPSPEAAPPQAGCLQLCWEPWQPGLHINQKCELRH |
| | | | KPQRGEASWALVGPLPLEALQYELCGLLPATAYTLQIR |
| | | | CIRWPLPGHWSDWSPSLELRTTERAPTVRLDTWWRQR |
| | | | QLDPRTVQLFWKPVPLEEDSGRIQGYVVSWRPSGQAG |
| | | | AILPLCNTTELSCTFHLPSEAQEVALVAYNSAGTSRPTP |
| | | | VVFSESRGPALTRLHAMARDPHSLWVGWEPPNPWPQG |
| | | | YVIEWGLGPPSASNSNKTWRMEQNGRATGFLLKENIRP |
| | | | FQLYEIIVTPLYQDTMGPSQHVYAYSQEMAPSHAPELH |
| | | | LKHIGKTWAQLEWVPEPPELGKSPLTHYTIFWTNAQNQ |
| | | | SFSAILNASSRGFVLHGLEPASLYHIHLMAASQAGATNS |
| | | | TVLTLMTLTPEGSELHIILGLFGLLLLLTCLCGTAWLCCS |
| | | | PNRKNPLWPSVPDPAHSSLGSWVPTIMEEDAFQLPGLG |
| | | | TPPITKLTVLEEDEKKPVPWESHNSSETCGLPTLVQTYV |
| | | | LQGDPRAVSTQPQSQSGTSDQVLYGQLLGSPTSPGPGH |
| | | | YLRCDSTQPLLAGLTPSPKSYENLWFQASPLGTLVTPAP |
| | | | SQEDDCVFGPLLNFPLLQGIRVHGMEALGSF (SEQ ID |
| | | | NO: 36) |
| IL3 | IL3Ra | 3563 | MVLLWLTLLLIALPCLLQTKEDPNPPITNLRMKAKAQQ |
| | | | LTWDLNRNVTDIECVKDADYSMPAVNNSYCQFGAISL |
| | | | CEVTNYTVRVANPPFSTWILFPENSGKPWAGAENLTCW |
| | | | IHDVDFLSCSWAVGPGAPADVQYDLYLNVANRRQQYE |
| | | | CLHYKTDAQGTRIGCREDDISRLSSGSQSSHILVRGRSA |
| | | | AFGIPCTDKFVVFSQIEILTPPNMTAKCNKTHSFMHWK |
| | | | MRSHFNRKFRYELQIQKRMQPVITEQVRDRTSFQLLNP |
| | | | GTYTVQIRARERVYEFLSAWSTPQRFECDQEEGANTRA |
| | | | WRTSLLIALGTLLALVCVFVICRRYLVMQRLFPRIPHMK |
| | | | DPIGDSFQNDKLVVWEAGKAGLEECLVTEVQVVQKT |
| | | | (SEQ ID NO: 37) |
| IL5 | IL5Ra | 3568 | MIIVAHVLLILLGATEILQADLLPDEKISLLPPVNFTIKVT |
| | | | GLAQVLLQWKPNPDQEQRNVNLEYQVKINAPKEDDYE |
| | | | TRITESKCVTILHKGFSASVRTILQNDHSLLASSWASAEL |
| | | | HAPPGSPGTSIVNLTCTTNTTEDNYSRLRSYQVSLHCTW |
| | | | LVGTDAPEDTQYFLYYRYGSWTEECQEYSKDTLGRNIA |
| | | | CWFPRTFILSKGRDWLAVLVNGSSKHSAIRPFDQLFAL |
| | | | HAIDQINPPLNVTAEIEGTRLSIQWEKPVSAFPIHCFDYE |
| | | | VKIHNTRNGYLQIEKLMTNAFISIIDDLSKYDVQVRAAV |
| | | | SSMCREAGLWSEWSQPIYVGNDEHKPLREWFVIVIMAT |
| | | | ICFILLILSLICKICHLWIKLFPPIPAPKSNIKDLFVTTNYE |
| | | | KAGSSETEIEVICYIEKPGVETLEDSVF (SEQ ID NO: 38) |
| GM-CSF | GMCSFRa | 1438 | MLLLVTSLLLCELPHPAFLLIPEKSDLRTVAPASSLNVRF |
| | | | DSRTMNLSWDCQENTTFSKCFLTDKKNRVVEPRLSNN |
| | | | ECSCTFREICLHEGVTFEVHVNTSQRGFQQKLLYPNSGR |
| | | | EGTAAQNFSCFIYNADLMNCTWARGPTAPRDVQYFLYI |
| | | | RNSKRRREIRCPYYIQDSGTHVGCHLDNLSGLTSRNYFL |
| | | | VNGTSREIGIQFFDSLLDTKKIERFNPPSNVTVRCNTTHC |
| | | | LVRWKQPRTYQKLSYLDFQYQLDVHRKNTQPGTENLL |
| | | | INVSGDLENRYNFPSSEPRAKHSVKIRAADVRILNWSS |
| | | | WSEAIEFGSDDGNLGSVYIYVLLIVGTLVCGIVLGFLFK |
| | | | RFLRIQRLFPPVPQIKDKLNDNHEVEDEIIWEEFTPEEGK |
| | | | GYREEVLTVKEIT (SEQ ID NO: 39) |
| IL3/5/GM-CSF | CSF2Rb | 1439 | MVLAQGLLSMALLALCWERSLAGAEETIPLQTLRCYN |
| | | | DYTSHITCRWADTQDAQRLVNVTLIRRVNEDLLEPVSC |
| | | | DLSDDMPWSACPHPRCVPRRCVIPCQSFVVTDVDYFSF |
| | | | QPDRPLGTRLTVTLTQHVQPPEPRDLQISTDQDHFLLTW |
| | | | SVALGSPQSHWLSPGDLEFEVVYKRLQDSWEDAAILLS |
| | | | NTSQATLGPEHLMPSSTYVARVRTRLAPGSRLSGRPSK |
| | | | WSPEVCWDSQPGDEAQPQNLECFFDGAAVLSCSWEVR |
| | | | KEVASSVSFGLFYKPSPDAGEEECSPVLREGLGSLHTRH |
| | | | HCQIPVPDPATHGQYIVSVQPRRAEKHIKSSVNIQMAPP |
| | | | SLNVTKDGDSYSLRWETMKMRYEHIDHTFEIQYRKDT |
| | | | ATWKDSKTETLQNAHSMALPALEPSTRYWARVRVRTS |
| | | | RTGYNGIWSEWSEARSWDTESVLPMWVLALIVIFLTIA |
| | | | VLLALRFCGIYGYRLRRKWEEKIPNPSKSHLFQNGSAEL |
| | | | WPPGSMSAFTSGSPPHQGPWGSRFPELEGVFPVGFGDS |
| | | | EVSPLTIEDPKHVCDPPSGPDTTPAASDLPTEQPPSPQPG |
| | | | PPAASHTPEKQASSFDFNGPYLGPPHSRSLPDILGQPEPP |
| | | | QEGGSQKSPPPGSLEYLCLPAGGQVQLVPLAQAMGPG |
| | | | QAVEVERRPSQGAAGSPSLESGGGPAPPALGPRVGGQD |
| | | | QKDSPVAIPMSSGDTEDPGVASGYVSSADLVFTPNSGA |
| | | | SSVSLVPSLGLPSDQTPSLCPGLASGPPGAPGPVKSGFEG |
| | | | YVELPPIEGRSPRSPRNNPVPPEAKSPVLNPGERPADVSP |
| | | | TSPQPEGLLVLQQVGDYCFLPGLGPGPLSLRSKPSSPGP |
| | | | GPEIKNLDQAFQVKKPPGQAVPQVPVIQLFKALKQQDY |
| | | | LSLPPWEVNKPGEVC (SEQ ID NO: 40) |
| LIF | LIFRb | 3977 | MMDIYVCLKRPSWMVDNKRMRTASNFQWLLSTFILLY |
| | | | LMNQVNSQKKGAPHDLKCVINNLQVWNCSWKAPSGT |
| | | | GRGTDYEVCIENRSRSCYQLEKTSIKIPALSHGDYEITIN |
| | | | SLHDFGSSTSKFTLNEQNVSLIPDTPEILNLSADFSTSTLY |
| | | | LKWNDRGSVFPHRSNVIWEIKVLRKESMELVKLVTHN |
| | | | TTLNGKDTLHHWSWASDMPLECAIHFVEIRCYIDNLHF |
| | | | SGLEEWSDWSPVKNISWIPDSQTKVFPQDKVILVGSDIT |
| | | | FCCVSQEKVLSALIGHTNCPLIHLDGENVAIKIRNISVSA |
| | | | SSGTNVVFTTEDNIFGTVIFAGYPPDTPQQLNCETHDLK |
| | | | EIICSWNPGRVTALVGPRATSYTLVESFSGKYVRLKRAE |
| | | | APTNESYQLLFQMLPNQEIYNFTLNAHNPLGRSQSTILV |
| | | | NITEKVYPHTPTSFKVKDINSTAVKLSWHLPGNFAKINF |
| | | | LCEIEIKKSNSVQEQRNVTIKGVENSSYLVALDKLNPYT |
| | | | LYTFRIRCSTETFWKWSKWSNKKQHLTTEASPSKGPDT |
| | | | WREWSSDGKNLIIYWKPLPINEANGKILSYNVSCSSDEE |
| | | | TQSLSEIPDPQHKAEIRLDKNDYIISVVAKNSVGSSPPSKI |
| | | | ASMEIPNDDLKIEQVVGMGKGILLTWHYDPNMTCDYV |
| | | | IKWCNSSRSEPCLMDWRKVPSNSTETVIESDEFRPGIRY |
| | | | NFFLYGCRNQGYQLLRSMIGYIEELAPIVAPNFTVEDTS |
| | | | ADSILVKWEDIPVEELRGFLRGYLFYFGKGERDTSKMR |
| | | | VLESGRSDIKVKNITDISQKTLRIADLQGKTSYHLVLRA |
| | | | YTDGGVGPEKSMYVVTKENSVGLIIAILIPVAVAVIVGV |
| | | | VTSILCYRKREWIKETFYPDIPNPENCKALQFQKSVCEG |
| | | | SSALKTLEMNPCTPNNVEVLETRSAFPKIEDTEIISPVAE |
| | | | RPEDRSDAEPENHVVVSYCPPIIEEEIPNPAADEAGGTA |
| | | | QVIYIDVQSMYQPQAKPEEEQENDPVGGAGYKPQMHL |
| | | | PINSTVEDIAAEEDLDKTAGYRPQANVNTWNLVSPDSP |
| | | | RSIDSNSEIVSFGSPCSINSRQFLIPPKDEDSPKSNGGGWS |
| | | | FTNFFQNKPND (SEQ ID NO: 41) |
| IL31 | IL31Ra | 133396 | MMWTWALWMLPSLCKFSLAALPAKPENISCVYYYRK |
| | | | NLTCTWSPGKETSYTQYTVKRTYAFGEKHDNCTTNSST |
| | | | SENRASCSFFLPRITIPDNYTIEVEAENGDGVIKSHMTY |
| | | | WRLENIAKTEPPKIFRVKPVLGIKRMIQIEWIKPELAPVS |
| | | | SDLKYTLRFRTVNSTSWMEVNFAKNRKDKNQTYNLTG |
| | | | LQPFTEYVIALRCAVKESKFWSDWSQEKMGMTEEEAP |
| | | | CGLELWRVLKPAEADGRRPVRLLWKKARGAPVLEKTL |
| | | | GYNIWYYPESNTNLTETMNTTNQQLELHLGGESFWVS |
| | | | MISYNSLGKSPVATLRIPAIQEKSFQCIEVMQACVAEDQ |
| | | | LVVKWQSSALDVNTWMIEWFPDVDSEPTTLSWESVSQ |
| | | | ATNWTIQQDKLKPFWCYNISVYPMLHDKVGEPYSIQA |
| | | | YAKEGVPSEGPETKVENIGVKTVTITWKEIPKSERKGIIC |
| | | | NYTIFYQAEGGKGFSKTVNSSILQYGLESLKRKTSYIVQ |
| | | | VMASTSAGGTNGTSINFKTLSFSVFEIILITSLIGGGLLILI |
| | | | ILTVAYGLKKPNKLTHLCWPTVPNPAESSIATWHGDDF |
| | | | KDKLNLKESDDSVNTEDRILKPCSTPSDKLVIDKLVVNF |
| | | | GNVLQEIFTDEARTGQENNLGGEKNGYVTCPFRPDCPL |
| | | | GKSFEELPVSPEIPPRKSQYLRSRMPEGTRPEAKEQLLFS |
| | | | GQSLVPDHLCEEGAPNPYLKNSVTAREFLVSEKLPEHT |
| | | | KGEV (SEQ ID NO: 42) |
| CNTF/CT-1 | CNTFR | 1271 | MAAPVPWACCAVLAAAAAVVYAQRHSPQEAPHVQYE |
| | | | RLGSDVTLPCGTANWDAAVTWRVNGTDLAPDLLNGS |
| | | | QLVLHGLELGHSGLYACFHRDSWHLRHQVLLHVGLPP |
| | | | REPVLSCRSNTYPKGFYCSWHLPTPTYIPNTFNVTVLHG |
| | | | SKIMVCEKDPALKNRCHIRYMHLFSTIKYKVSISVSNAL |
| | | | GHNATAITFDEFTIVKPDPPENVVARPVPSNPRRLEVTW |
| | | | QTPSTWPDPESFPLKFFLRYRPLILDQWQHVELSDGTAH |
| | | | TITDAYAGKEYIIQVAAKDNEIGTWSDWSVAAHATPWT |
| | | | EEPRHLTTEAQAAETTTSTTSSLAPPPTTKICDPGELGSG |
| | | | GGPSAPFLVSVPITLALAAAAATASSLLI (SEQ ID NO: |
| | | | 43) |
| IL27 | IL27Ra | 9466 | MRGGRGAPFWLWPLPKLALLPLLWVLFQRTRPQGSAG |
| | | | PLQCYGVGPLGDLNCSWEPLGDLGAPSELHLQSQKYRS |
| | | | NKTQTVAVAAGRSWVAIPREQLTMSDKLLVWGTKAG |
| | | | QPLWPPVFVNLETQMKPNAPRLGPDVDFSEDDPLEATV |
| | | | HWAPPTWPSHKVLICQFHYRRCQEAAWTLLEPELKTIP |
| | | | LTPVEIQDLELATGYKVYGRCRMEKEEDLWGEWSPILS |
| | | | FQTPPSAPKDVWVSGNLCGTPGGEEPLLLWKAPGPCVQ |
| | | | VSYKVWFWVGGRELSPEGITCCCSLIPSGAEWARVSAV |
| | | | NATSWEPLTNLSLVCLDSASAPRSVAVSSIAGSTELLVT |
| | | | WQPGPGEPLEHVVDWARDGDPLEKLNWVRLPPGNLSA |
| | | | LLPGNFTVGVPYRITVTAVSASGLASASSVWGFREELAP |
| | | | LVGPTLWRLQDAPPGTPAIAWGEVPRHQLRGHLTHYT |
| | | | LCAQSGTSPSVCMNVSGNTQSVTLPDLPWGPCELWVT |
| | | | ASTIAGQGPPGPILRLHLPDNTLRWKVLPGILFLWGLFL |
| | | | LGCGLSLATSGRCYHLRHKVLPRWVWEKVPDPANSSS |
| | | | GQPHMEQVPEAQPLGDLPILEVEEMEPPPVMESSQPAQ |
| | | | ATAPLDSGYEKHELPTPEELGLLGPPRPQVLA (SEQ ID |
| | | | NO: 44) |
| EPO | EPOR | 2057 | MDHLGASLWPQVGSLCLLLAGAAWAPPPNLPDPKFES |
| | | | KAALLAARGPEELLCFTERLEDLVCFWEEAASAGVGPG |
| | | | NYSFSYQLEDEPWKLCRLHQAPTARGAVRFWCSLPTA |
| | | | DTSSFVPLELRVTAASGAPRYHRVIHINEVVLLDAPVGL |
| | | | VARLADESGHVVLRWLPPPETPMTSHIRYEVDVSAGNG |
| | | | AGSVQRVEILEGRTECVLSNLRGRTRYTFAVRARMAEP |
| | | | SFGGFWSAWSEPVSLLTPSDLDPLILTLSLILVVILVLLT |
| | | | VLALLSHRRALKQKIWPGIPSPESEFEGLFTTHKGNFQL |
| | | | WLYQNDGCLWWSPCTPFTEDPPASLEVLSERCWGTMQ |
| | | | AVEPGTDDEGPLLEPVGSEHAQDTYLVLDKWLLPRNPP |
| | | | SEDLPGPGGSVDIVAMDEGSEASSCSSALASKPSPEGAS |
| | | | AASFEYTILDPSSQLLRPWTLCPELPPTPPHLKYLYLVVS |
| | | | DSGISTDYSSGDSQGAQGGLSDGPYSNPYENSLIPAAEP |
| | | | LPPSYVACS (SEQ ID NO: 45) |
| GH | GHR | 2690 | MDLWQLLLTLALAGSSDAFSGSEATAAILSRAPWSLQS |
| | | | VNPGLKTNSSKEPKFTKCRSPERETFSCHWTDEVHHGT |
| | | | KNLGPIQLFYTRRNTQEWTQEWKECPDYVSAGENSCY |
| | | | FNSSFTSIWIPYCIKLTSNGGTVDEKCFSVDEIVQPDPPIA |
| | | | LNWTLLNVSLTGIHADIQVRWEAPRNADIQKGWMVLE |
| | | | YELQYKEVNETKWKMMDPILTTSVPVYSLKVDKEYEV |
| | | | RVRSKQRNSGNYGEFSEVLYVTLPQMSQFTCEEDFYFP |
| | | | WLLIIIFGIFGLTVMLFVFLFSKQQRIKMLILPPVPVPKIK |
| | | | GIDPDLLKEGKLEEVNTILAIHDSYKPEFHSDDSWVEFIE |
| | | | LDIDEPDEKTEESDTDRLLSSDHEKSHSNLGVKDGDSG |
| | | | RTSCCEPDILETDFNANDIHEGTSEVAQPQRLKGEADLL |
| | | | CLDQKNQNNSPYHDACPATQQPSVIQAEKNKPQPLPTE |
| | | | GAESTHQAAHIQLSNPSSLSNIDFYAQVSDITPAGSVVLS |
| | | | PGQKNKAGMSQCDMHPEMVSLCQENFLMDNAYFCEA |
| | | | DAKKCIPVAPHIKVESHIQPSLNQEDIYITTESLTTAAGR |
| | | | PGTGEHVPGSEMPVPDYTSIHIVQSPQGLILNATALPLPD |
| | | | KEFLSSCGYVSTDQLNKIMP (SEQ ID NO: 46) |
| PRL | PRLR | 5618 | MKENVASATVFTLLLFLNTCLLNGQLPPGKPEIFKCRSP |
| | | | NKETFTCWWRPGTDGGLPTNYSLTYHREGETLMHECP |
| | | | DYITGGPNSCHFGKQYTSMWRTYIMMVNATNQMGSSF |
| | | | SDELYVDVTYIVQPDPPLELAVEVKQPEDRKPYLWIKW |
| | | | SPPTLIDLKTGWFTLLYEIRLKPEKAAEWEIHFAGQQTE |
| | | | FKILSLHPGQKYLVQVRCKPDHGYWSAWSPATFIQIPSD |
| | | | FTMNDTTVWISVAVLSAVICLIIVWAVALKGYSMVTCI |
| | | | FPPVPGPKIKGFDAHLLEKGKSEELLSALGCQDFPPTSD |
| | | | YEDLLVEYLEVDDSEDQHLMSVHSKEHPSQGMKPTYL |
| | | | DPDTDSGRGSCDSPSLLSEKCEEPQANPSTFYDPEVIEKP |
| | | | ENPETTHTWDPQCISMEGKIPYFHAGGSKCSTWPLPQPS |
| | | | QHNPRSSYHNITDVCELAVGPAGAPATLLNEAGKDALK |
| | | | SSQTIKSREEGKATQQREVESFHSETDQDTPWLLPQEKT |
| | | | PFGSAKPLDYVEIHKVNKDGALSLLPKQRENSGKPKKP |
| | | | GTPENNKEYAKVSGVMDNNILVLVPDPHAKNVACFEE |
| | | | SAKEAPPSLEQNQAEKALANFTATSSKCRLQLGGLDYL |
| | | | DPACFTHSFH (SEQ ID NO: 47) |
| IFNα/β/ω/ε/κ | IFNAR2 | 3455 | MLLSQNAFIFRSLNLVLMVYISLVFGISYDSPDYTDESC |
| | | | TFKISLRNFRSILSWELKNHSIVPTHYTLLYTIMSKPEDL |
| | | | KVVKNCANTTRSFCDLTDEWRSTHEAYVTVLEGFSGN |
| | | | TTLFSCSHNFWLAIDMSFEPPEFEIVGFTNHINVMVKFPS |
| | | | IVEEELQFDLSLVIEEQSEGIVKKHKPEIKGNMSGNFTYII |
| | | | DKLIPNTNYCVSVYLEHSDEQAVIKSPLKCTLLPPGQES |
| | | | ESAESAKIGGIITVFLIALVLTSTIVTLKWIGYICLRNSLP |
| | | | KVLNFHNFLAWPFPNLPPLEAMDMVEVIYINRKKKVW |
| | | | DYNYDDESDSDTEAAPRTSGGGYTMHGLTVRPLGQAS |
| | | | ATSTESQLIDPESEEEPDLPEVDVELPTMPKDSPQQLELL |
| | | | SGPCERRKSPLQDPFPEEDYSSTEGSGGRITFNVDLNSVF |
| | | | LRVLDDEDSDDLEAPLMLSSHLEEMVDPEDPDNVQSN |
| | | | HLLASGEGTQPTFPSPSSEGLWSEDAPSDQSDTSESDVD |
| | | | LGDGYIMR (SEQ ID NO: 48) |
| IFNγ | IFNGR1 | 3459 | MALLFLLPLVMQGVSRAEMGTADLGPSSVPTPTNVTIE |
| | | | SYNMNPIVYWEYQIMPQVPVFTVEVKNYGVKNSEWID |
| | | | ACINISHHYCNISDHVGDPSNSLWVRVKARVGQKESAY |
| | | | AKSEEFAVCRDGKIGPPKLDIRKEEKQIMIDIFHPSVFVN |
| | | | GDEQEVDYDPETTCYIRVYNVYVRMNGSEIQYKILTQK |
| | | | EDDCDEIQCQLAIPVSSLNSQYCVSAEGVLHVWGVTTE |
| | | | KSKEVCITIFNSSIKGSLWIPVVAALLLFLVLSLVFICFYI |
| | | | KKINPLKEKSIILPKSLISVVRSATLETKPESKYVSLITSY |
| | | | QPFSLEKEVVCEEPLSPATVPGMHTEDNPGKVEHTEELS |
| | | | SITEVVTTEENIPDVVPGSHLTPIERESSSPLSSNQSEPGSI |
| | | | ALNSYHSRNCSESDHSRNGFDTDSSCLESHSSLSDSEFPP |
| | | | NNKGEIKTEGQELITVIKAPTSFGYDKPHVLVDLLVDDS |
| | | | GKESLIGYRPTEDSKEFS (SEQ ID NO: 49) |
| IFNλ1/λ2/λ3 | IL28R | 163702 | MAGPERWGPLLLCLLQAAPGRPRLAPPQNVTLLSQNFS |
| | | | VYLTWLPGLGNPQDVTYFVAYQSSPTRRRWREVEECA |
| | | | GTKELLCSMMCLKKQDLYNKFKGRVRTVSPSSKSPWV |
| | | | ESEYLDYLFEVEPAPPVLVLTQTEEILSANATYQLPPCM |
| | | | PPLDLKYEVAFWKEGAGNKTLFPVTPHGQPVQITLQPA |
| | | | ASEHHCLSARTIYTFSVPKYSKFSKPTCFLLEVPEANWA |
| | | | FLVLPSLLILLLVIAAGGVIWKTLMGNPWFQRAKMPRA |
| | | | LDFSGHTHPVATFQPSRPESVNDLFLCPQKELTRGVRPT |
| | | | PRVRAPATQQTRWKKDLAEDEEEEDEEDTEDGVSFQP |
| | | | YIEPPSFLGQEHQAPGHSEAGGVDSGRPRAPLVPSEGSS |
| | | | AWDSSDRSWASTVDSSWDRAGSSGYLAEKGPGQGPG |
| | | | GDGHQESLPPPEFSKDSGFLEELPEDNLSSWATWGTLPP |
| | | | EPNLVPGGPPVSLQTLTFCWESSPEEEEEARESEIEDSDA |
| | | | GSWGAESTQRTEDRGRTLGHYMAR (SEQ ID NO: 50) |
| IL26/19/20/24 | IL20Ra | 53832 | MRAPGRPALRPLPLPPLLLLLLAAPWGRAVPCVSGGLP |
| | | | KPANITFLSINMKNVLQWTPPEGLQGVKVTYTVQYFIY |
| | | | GQKKWLNKSECRNINRTYCDLSAETSDYEHQYYAKVK |
| | | | AIWGTKCSKWAESGRFYPFLETQIGPPEVALTTDEKSIS |
| | | | VVLTAPEKWKRNPEDLPVSMQQIYSNLKYNVSVLNTK |
| | | | SNRTWSQCVTNHTLVLTWLEPNTLYCVHVESFVPGPPR |
| | | | RAQPSEKQCARTLKDQSSEFKAKIIFWYVLPVSITVFLFS |
| | | | VMGYSIYRYIHVGKEKHPANLILIYGNEFDKRFFVPAEK |
| | | | IVINFITLNISDDSKISHQDMSLLGKSSDVSSLNDPQPSG |
| | | | NLRPPQEEEEVKHLGYASHLMEIFCDSEENTEGTSLTQQ |
| | | | ESLSRTIPPDKTVIEYEYDVRTTDICAGPEEQELSLQEEV |
| | | | STQGTLLESQAALAVLGPQTLQYSYTPQLQDLDPLAQE |
| | | | HTDSEEGPEEEPSTTLVDWDPQTGRLCIPSLSSFDQDSE |
| | | | GCEPSEGDGLGEEGLLSRLYEEPAPDRPPGENETYLMQ |
| | | | FMEEWGLYVQMEN (SEQ ID NO: 51) |
| IL22/20/24 | IL22R | 58985 | MRTLLTILTVGSLAAHAPEDPSDLLQHVKFQSSNFENIL |
| | | | TWDSGPEGTPDTVYSIEYKTYGERDWVAKKGCQRITR |
| | | | KSCNLTVETGNLTELYYARVTAVSAGGRSATKMTDRF |
| | | | SSLQHTTLKPPDVTCISKVRSIQMIVHPTPTPIRAGDGHR |
| | | | LTLEDIFHDLFYHLELQVNRTYQMHLGGKQREYEFFGL |
| | | | TPDTEFLGTIMICVPTWAKESAPYMCRVKTLPDRTWTY |
| | | | SFSGAFLFSMGFLVAVLCYLSYRYVTKPPAPPNSLNVQ |
| | | | RVLTFQPLRFIQEHVLIPVEDLSGPSSLAQPVQYSQIRVS |
| | | | GPREPAGAPQRHSLSEITYLGQPDISILQPSNVPPPQILSP |
| | | | LSYAPNAAPEVGPPSYAPQVTPEAQFPFYAPQAISKVQP |
| | | | SSYAPQATPDSWPPSYGVCMEGSGKDSPTGTLSSPKHL |
| | | | RPKGQLQKEPPAGSCMLGGLSLQEVTSLAMEESQEAKS |
| | | | LHQPLGICTDRTSDPNVLHSGEEGTPQYLKGQLPLLSSV |
| | | | QIEGHPMSLPLQPPSRPCSPSDQGPSPWGLLESLVCPKD |
| | | | EAKSPAPETSDLEQPTELDSLFRGLALTVQWES (SEQ ID |
| | | | NO: 52) |
| STAT1 | minimal STAT1 | N/A | pYxxP (SEQ ID NO: 53) |
| | binding motif | | |
| STAT3 | minimal STAT3 | N/A | pYxxQ (SEQ ID NO: 54) |
| | binding motif | | |
| STAT5 | minimal | N/A | pYxxL (SEQ ID NO: 55) |
| | STAT5a binding | | |
| | motif | | |
| STAT6 | minimal STAT6 | N/A | pYxxF (SEQ ID NO: 56) |
| | binding motif | | |
| CD40 | CD40 | 958 | MVRLPLQCVLWGCLLTAVHPEPPTACREKQYLINSQCC |
| | | | SLCQPGQKLVSDCTEFTETECLPCGESEFLDTWNRETHC |
| | | | HQHKYCDPNLGLRVQQKGTSETDTICTCEEGWHCTSE |
| | | | ACESCVLHRSCSPGFGVKQIATGVSDTICEPCPVGFFSN |
| | | | VSSAFEKCHPWTSCETKDLVVQQAGTNKTDVVCGPQD |
| | | | RLRALVVIPIIFGILFAILLVLVFIKKVAKKPTNKAPHPKQ |
| | | | EPQEINFPDDLPGSNTAAPVQETLHGCQPVTQEDGKESR |
| | | | ISVQERQ (SEQ ID NO: 57) |
| 4-1BB | 4-1BB | 3604 | MGNSCYNIVATLLLVLNFERTRSLQDPCSNCPAGTFCD |
| | | | NNRNQICSPCPPNSFSSAGGQRTCDICRQCKGVFRTRKE |
| | | | CSSTSNAECDCTPGFHCLGAGCSMCEQDCKQGQELTK |
| | | | KGCKDCCFGTFNDQKRGICRPWTNCSLDGKSVLVNGT |
| | | | KERDVVCGPSPADLSPGASSVTPPAPAREPGHSPQIISFF |
| | | | LALTSTALLFLLFFLTLRFSVVKRGRKKLLYIFKQPFMR |
| | | | PVQTTQEEDGCSCRFPEEEEGGCEL (SEQ ID NO: 58) |
| TLR4 | MyD88 | 4615 | MAAGGPGAGSAAPVSSTSSLPLAALNMRVRRRLSLFLN |
| | | | VRTQVAADWTALAEEMDFEYLEIRQLETQADPTGRLL |
| | | | DAWQGRPGASVGRLLELLTKLGRDDVLLELGPSIEEDC |
| | | | QKYILKQQQEEAEKPLQVAAVDSSVPRTAELAGITTLD |
| | | | DPLGHMPERFDAFICYCPSDIQFVQEMIRQLEQTNYRLK |
| | | | LCVSDRDVLPGTCVWSIASELIEKRCRRMVVVVSDDYL |
| | | | QSKECDFQTKFALSLSPGAHQKRLIPIKYKAMKKEFPSI |
| | | | LRFITVCDYTNPCTKSWFWTRLAKALSLP (SEQ ID NO: |
| | | | 59) |
| OX40 | OX40 | 7293 | MCVGARRLGRGPCAALLLLGLGLSTVTGLHCVGDTYP |
| | | | SNDRCCHECRPGNGMVSRCSRSQNTVCRPCGPGFYND |
| | | | VVSSKPCKPCTWCNLRSGSERKQLCTATQDTVCRCRA |
| | | | GTQPLDSYKPGVDCAPCPPGHFSPGDNQACKPWTNCTL |
| | | | AGKHTLQPASNSSDAICEDRDPPATQPQETQGPPARPIT |
| | | | VQPTEAWPRTSQGPSTRPVEVPGGRAVAAILGLGLVLG |
| | | | LLGPLAILLALYLLRRDQRLPPDAHKPPGGGSFRTPIQEE |
| | | | QADAHSTLAKI (SEQ ID NO: 60) |
| CD30 | CD30 | 943 | MRVLLAALGLLFLGALRAFPQDRPFEDTCHGNPSHYYD |
| | | | KAVRRCCYRCPMGLFPTQQCPQRPTDCRKQCEPDYYL |
| | | | DEADRCTACVTCSRDDLVEKTPCAWNSSRVCECRPGM |
| | | | FCSTSAVNSCARCFFHSVCPAGMIVKFPGTAQKNTVCE |
| | | | PASPGVSPACASPENCKEPSSGTIPQAKPTPVSPATSSAS |
| | | | TMPVRGGTRLAQEAASKLTRAPDSPSSVGRPSSDPGLSP |
| | | | TQPCPEGSGDCRKQCEPDYYLDEAGRCTACVSCSRDDL |
| | | | VEKTPCAWNSSRTCECRPGMICATSATNSCARCVPYPIC |
| | | | AAETVTKPQDMAEKDTTFEAPPLGTQPDCNPTPENGEA |
| | | | PASTSPTQSLLVDSQASKTLPIPTSAPVALSSTGKPVLDA |
| | | | GPVLFWVILVLVVVVGSSAFLLCHRRACRKRIRQKLHL |
| | | | CYPVQTSQPKLELVDSRPRRSSTQLRSGASVTEPVAEER |
| | | | GLMSQPLMETCHSVGAAYLESLPLQDASPAGGPSSPRD |
| | | | LPEPRVSTEHTNNKIEKIYIMKADTVIVGTVKAELPEGR |
| | | | GLAGPAEPELEEELEADHTPHYPEQETEPPLGSCSDVML |
| | | | SVEEEGKEDPLPTAASGK (SEQ ID NO: 61) |
| TNFa | TNFR1 | 7132 | MGLSTVPDLLLPLVLLELLVGIYPSGVIGL VPHLGDREK |
| | | | RDSVCPQGKYIHPQNNSICCTKCHKGTYLYNDCPGPGQ |
| | | | DTDCRECESGSFTASENHLRHCLSCSKCRKEMGQVEISS |
| | | | CTVDRDTVCGCRKNQYRHYWSENLFQCFNCSLCLNGT |
| | | | VHLSCQEKQNTVCTCHAGFFLRENECVSCSNCKKSLEC |
| | | | TKLCLPQIENVKGTEDSGTTVLLPLVIFFGLCLLSLLFIG |
| | | | LMYRYQRWKSKLYSIVCGKSTPEKEGELEGTTTKPLAP |
| | | | NPSFSPTPGFTPTLGFSPVPSSTFTSSSTYTPGDCPNFAAP |
| | | | RREVAPPYQGADPILATALASDPIPNPLQKWEDSAHKP |
| | | | QSLDTDDPATLYAVVENVPPLRWKEFVRRLGLSDHEID |
| | | | RLELQNGRCLREAQYSMLATWRRRTPRREATLELLGR |
| | | | VLRDMDLLGCLEDIEEALCGPAALPPAPSLLR (SEQ ID |
| | | | NO: 62) |
| TNFa | TNFR2 | 7133 | MAPVAVWAALAVGLELWAAAHALPAQVAFTPYAPEP |
| | | | GSTCRLREYYDQTAQMCCSKCSPGQHAKVFCTKTSDT |
| | | | VCDSCEDSTYTQLWNWVPECLSCGSRCSSDQVETQAC |
| | | | TREQNRICTCRPGWYCALSKQEGCRLCAPLRKCRPGFG |
| | | | VARPGTETSDVVCKPCAPGTFSNTTSSTDICRPHQICNV |
| | | | VAIPGNASMDAVCTSTSPTRSMAPGAVHLPQPVSTRSQ |
| | | | HTQPTPEPSTAPSTSFLLPMGPSPPAEGSTGDFALPVGLI |
| | | | VGVTALGLLIIGVVNCVIMTQVKKKPLCLQREAKVPHL |
| | | | PADKARGTQGPEQQHLLITAPSSSSSSLESSASALDRRA |
| | | | PTRNQPQAPGVEASGAGEARASTGSSDSSPGGHGTQVN |
| | | | VTCIVNVCSSSDHSSQCSSQASSTMGDTDSSPSESPKDE |
| | | | QVPFSKEECAFRSQLETPETLLGSTEEKPLPLGVPDAGM |
| | | | KPS (SEQ ID NO: 63) |
| FLT3L | FLT3 | 2322 | MPALARDGGQLPLLVVFSAMIFGTITNQDLPVIKCVLIN |
| | | | HKNNDSSVGKSSSYPMVSESPEDLGCALRPQSSGTVYE |
| | | | AAAVEVDVSASITLQVLVDAPGNISCLWVFKHSSLNCQ |
| | | | PHFDLQNRGVVSMVILKMTETQAGEYLLFIQSEATNYTI |
| | | | LFTVSIRNTLLYTLRRPYFRKMENQDALVCISESVPEPIV |
| | | | EWVLCDSQGESCKEESPAVVKKEEKVLHELFGTDIRCC |
| | | | ARNELGRECTRLFTIDLNQTPQTTLPQLFLKVGEPLWIR |
| | | | CKAVHVNHGFGLTWELENKALEEGNYFEMSTYSTNRT |
| | | | MIRILFAFVSSVARNDTGYYTCSSSKHPSQSALVTIVEK |
| | | | GFINATNSSEDYEIDQYEEFCFSVRFKAYPQIRCTWTFSR |
| | | | KSFPCEQKGLDNGYSISKFCNHKHQPGEYIFHAENDDA |
| | | | QFTKMFTLNIRRKPQVLAEASASQASCFSDGYPLPSWT |
| | | | WKKCSDKSPNCTEEITEGVWNRKANRKVFGQWVSSST |
| | | | LNMSEAIKGFLVKCCAYNSLGTSCETILLNSPGPFPFIQD |
| | | | NISFYATIGVCLLFIVVLTLLICHKYKKQFRYESQLQMV |
| | | | QVTGSSDNEYFYVDFREYEYDLKWEFPRENLEFGKVL |
| | | | GSGAFGKVMNATAYGISKTGVSIQVAVKMLKEKADSS |
| | | | EREALMSELKMMTQLGSHENIVNLLGACTLSGPIYLIFE |
| | | | YCCYGDLLNYLRSKREKFHRTWTEIFKEHNFSFYPTFQS |
| | | | HPNSSMPGSREVQIHPDSDQISGLHGNSFHSEDEIEYEN |
| | | | QKRLEEEEDLNVLTFEDLLCFAYQVAKGMEFLEFKSCV |
| | | | HRDLAARNVLVTHGKVVKICDFGLARDIMSDSNYVVR |
| | | | GNARLPVKWMAPESLFEGIYTIKSDVWSYGILLWEIFSL |
| | | | GVNPYPGIPVDANFYKLIQNGFKMDQPFYATEEIYIIMQ |
| | | | SCWAFDSRKRPSFPNLTSFLGCQLADAEEAMYQNVDG |
| | | | RVSECPHTYQNRRPFSREMDLGLLSPQAQVEDS (SEQ |
| | | | ID NO: 64) |
| TREM1 | TREM1 | 54210 | MRKTRLWGLLWMLFVSELRAATKLTEEKYELKEGQTL |
| | | | DVKCDYTLEKFASSQKAWQIIRDGEMPKTLACTERPSK |
| | | | NSHPVQVGRIILEDYHDHGLLRVRMVNLQVEDSGLYQ |
| | | | CVIYQPPKEPHMLFDRIRLVVTKGFSGTPGSNENSTQNV |
| | | | YKIPPTTTKALCPLYTSPRTVTQAPPKSTADVSTPDSEIN |
| | | | LTNVTDIIRVPVFNIVILLAGGFLSKSLVFSVLFAVTLRSF |
| | | | VP (SEQ ID NO: 65) |
| TREM2 | TREM2 | 54209 | MEPLRLLILLFVTELSGAHNTTVFQGVAGQSLQVSCPY |
| | | | DSMKHWGRRKAWCRQLGEKGPCQRVVSTHNLWLLSF |
| | | | LRRWNGSTAITDDTLGGTLTITLRNLQPHDAGLYQCQS |
| | | | LHGSEADTLRKVLVEVLADPLDHRDAGDLWFPGESESF |
| | | | EDAHVEHSISRSLLEGEIPFPPTSILLLLACIFLIKILAASA |
| | | | LWAAAWHGQKPGTHPPSELDCGHDPGYQLQTLPGLRD |
| | | | T (SEQ ID NO: 66) |
| IFNλ | IFNLR1 | 163702 | KTLMGNPWFQRAKMPRALDFSGHTHPVATFQPSRPES |
| | | | VNDLFLCPQKELTRGVRPTPRVRAPATQQTRWKKDLA |
| | | | EDEEEEDEEDTEDGVSFQPYIEPPSFLGQEHQAPGHSEA |
| | | | GGVDSGRPRAPLVPSEGSSAWDSSDRSWASTVDSSWD |
| | | | RAGSSGYLAEKGPGQGPGGDGHQESLPPPEFSKDSGFL |
| | | | EELPEDNLSSWATWGTLPPEPNLVPGGPPVSLQTLTFC |
| | | | WESSPEEEEEARESEIEDSDAGSWGAESTQRTEDRGRTL |
| | | | GHYMAR (SEQ ID NO: 67) |
| IFNα/β/ω/ε/κ | IFNAR2 | 3455 | KWIGYICLRNSLPKVLNFHNFLAWPFPNLPPLEAMDMV |
| | | | EVIYINRKKKVWDYNYDDESDSDTEAAPRTSGGGYTM |
| | | | HGLTVRPLGQASATSTESQLIDPESEEEPDLPEVDVELPT |
| | | | MPKDSPQQLELLSGPCERRKSPLQDPFPEEDYSSTEGSG |
| | | | GRITFNVDLNSVFLRVLDDEDSDDLEAPLMLSSHLEEM |
| | | | VDPEDPDNVQSNHLLASGEGTQPTFPSPSSEGLWSEDAP |
| | | | SDQSDTSESDVDLGDGYIMR (SEQ ID NO: 68) |
| IFNγ | IFNGR1 | 3459 | CFYIKKINPLKEKSIILPKSLISVVRSATLETKPESKYVSLI |
| | | | TSYQPFSLEKEVVCEEPLSPATVPGMHTEDNPGKVEHT |
| | | | EELSSITEVVTTEENIPDVVPGSHLTPIERESSSPLSSNQS |
| | | | EPGSIALNSYHSRNCSESDHSRNGFDTDSSCLESHSSLSD |
| | | | SEFPPNNKGEIKTEGQELITVIKAPTSFGYDKPHVLVDLL |
| | | | VDDSGKESLIGYRPTEDSKEFS (SEQ ID NO: 69) |
| N/A | STAT1 minimal | 3459 | PTSFGYDKPHVL (SEQ ID NO: 70) |
| | binding (from | | |
| | IFNGR1) | | |
| IL10 | IL10Ra | 3587 | KTLMGNPWFQRAKMPRALDFSGHTHPVATFQPSRPES |
| | | | VNDLFLCPQKELTRGVRPTPRVRAPATQQTRWKKDLA |
| | | | EDEEEEDEEDTEDGVSFQPYIEPPSFLGQEHQAPGHSEA |
| | | | GGVDSGRPRAPLVPSEGSSAWDSSDRSWASTVDSSWD |
| | | | RAGSSGYLAEKGPGQGPGGDGHQESLPPPEFSKDSGFL |
| | | | EELPEDNLSSWATWGTLPPEPNLVPGGPPVSLQTLTFC |
| | | | WESSPEEEEEARESEIEDSDAGSWGAESTQRTEDRGRTL |
| | | | GHYMAR (SEQ ID NO: 71) |
| N/A | STAT3 minimal | 3587 | LAKGYLKQDPL (SEQ ID NO: 72) |
| | binding (from | | |
| | IL10Ra) | | |
| TGF-β | TGFbR2 | 7048 | CYRVNRQQKLSTWETGKTRKLMEFSEHCAIILEDDRSD |
| | (ΔS199) | | ISSTCANNINHNTELLPIELDTLVGKGRFAEVYKAKLKQ |
| | | | NTSEQFETVAVKIFPYEEYASWKTEKDIFSDINLKHENIL |
| | | | QFLTAEERKTELGKQYWLITAFHAKGNLQEYLTRHVIS |
| | | | WEDLRKLGSSLARGIAHLHSDHTPCGRPKMPIVHRDLK |
| | | | SSNILVKNDLTCCLCDFGLSLRLDPTLSVDDLANSGQV |
| | | | GTARYMAPEVLESRMNLENVESFKQTDVYSMALVLW |
| | | | EMTSRCNAVGEVKDYEPPFGSKVREHPCVESMKDNVL |
| | | | RDRGRPEIPSFWLNHQGIQMVCETLTECWDHDPEARLT |
| | | | AQCVAERFSELEHLDRLSGRSCSEEKIPEDGSLNTTK |
| | | | (SEQ ID NO: 73) |
| Tyro3 | Tyro3 | 7301 | MALRRSMGRPGLPPLPLPPPPRLGLLLAALASLLLPESA |
| | | | AAGLKLMGAPVKLTVSQGQPVKLNCSVEGMEEPDIQW |
| | | | VKDGAVVQNLDQLYIPVSEQHWIGFLSLKSVERSDAGR |
| | | | YWCQVEDGGETEISQPVWLTVEGVPFFTVEPKDLAVPP |
| | | | NAPFQLSCEAVGPPEPVTIVWWRGTTKIGGPAPSPSVLN |
| | | | VTGVTQSTMFSCEAHNLKGLASSRTATVHLQALPAAPF |
| | | | NITVTKLSSSNASVAWMPGADGRALLQSCTVQVTQAP |
| | | | GGWEVLAVVVPVPPFTCLLRDLVPATNYSLRVRCANA |
| | | | LGPSPYADWVPFQTKGLAPASAPQNLHAIRTDSGLILE |
| | | | WEEVIPEAPLEGPLGPYKLSWVQDNGTQDELTVEGTRA |
| | | | NLTGWDPQKDLIVRVCVSNAVGCGPWSQPLVVSSHDR |
| | | | AGQQGPPHSRTSWVPVVLGVLTALVTAAALALILLRKR |
| | | | RKETRFGQAFDSVMARGEPAVHFRAARSFNRERPERIE |
| | | | ATLDSLGISDELKEKLEDVLIPEQQFTLGRMLGKGEFGS |
| | | | VREAQLKQEDGSFVKVAVKMLKADIIASSDIEEFLREA |
| | | | ACMKEFDHPHVAKLVGVSLRSRAKGRLPIPMVILPFMK |
| | | | HGDLHAFLLASRIGENPFNLPLQTLIRFMVDIACGMEYL |
| | | | SSRNFIHRDLAARNCMLAEDMTVCVADFGLSRKIYSGD |
| | | | YYRQGCASKLPVKWLALESLADNLYTVQSDVWAFGV |
| | | | TMWEIMTRGQTPYAGIENAEIYNYLIGGNRLKQPPECM |
| | | | EDVYDLMYQCWSADPKQRPSFTCLRMELENILGQLSV |
| | | | LSASQDPLYINIERAEEPTAGGSLELPGRDQPYSGAGDG |
| | | | SGMGAVGGTPSDCRYILTPGGLAEQPGQAEHQPESPLN |
| | | | ETQRLLLLQQGLLPHSSC (SEQ ID NO: 74) |
| Axl | Axl | 558 | MAWRCPRMGRVPLAWCLALCGWACMAPRGTQAEESP |
| | | | FVGNPGNITGARGLTGTLRCQLQVQGEPPEVHWLRDG |
| | | | QILELADSTQTQVPLGEDEQDDWIVVSQLRITSLQLSDT |
| | | | GQYQCLVFLGHQTFVSQPGYVGLEGLPYFLEEPEDRTV |
| | | | AANTPFNLSCQAQGPPEPVDLLWLQDAVPLATAPGHGP |
| | | | QRSLHVPGLNKTSSFSCEAHNAKGVTTSRTATITVLPQQ |
| | | | PRNLHLVSRQPTELEVAWTPGLSGIYPLTHCTLQAVLSD |
| | | | DGMGIQAGEPDPPEEPLTSQASVPPHQLRLGSLHPHTPY |
| | | | HIRVACTSSQGPSSWTHWLPVETPEGVPLGPPENISATR |
| | | | NGSQAFVHWQEPRAPLQGTLLGYRLAYQGQDTPEVLM |
| | | | DIGLRQEVTLELQGDGSVSNLTVCVAAYTAAGDGPWS |
| | | | LPVPLEAWRPGQAQPVHQLVKEPSTPAFSWPWWYVLL |
| | | | GAVVAAACVLILALFLVHRRKKETRYGEVFEPTVERGE |
| | | | LVVRYRVRKSYSRRTTEATLNSLGISEELKEKLRDVMV |
| | | | DRHKVALGKTLGEGEFGAVMEGQLNQDDSILKVAVKT |
| | | | MKIAICTRSELEDELSEAVCMKEFDHPNVMRLIGVCFQ |
| | | | GSERESFPAPVVILPFMKHGDLHSFLLYSRLGDQPVYLP |
| | | | TQMLVKFMADIASGMEYLSTKRFIHRDLAARNCMLNE |
| | | | NMSVCVADFGLSKKIYNGDYYRQGRIAKMPVKWIAIES |
| | | | LADRVYTSKSDVWSFGVTMWEIATRGQTPYPGVENSEI |
| | | | YDYLRQGNRLKQPADCLDGLYALMSRCWELNPQDRPS |
| | | | FTELREDLENTLKALPPAQEPDEILYVNMDEGGGYPEPP |
| | | | GAAGGADPPTQPDPKDSCSCLTAAEVHPAGRYVLCPST |
| | | | TPSPAQPADRGSPAAPGQEDGA (SEQ ID NO: 75) |
| MerTK | MerTK | 10461 | MGPAPLPLLLGLFLPALWRRAITEAREEAKPYPLFPGPF |
| | | | PGSLQTDHTPLLSLPHASGYQPALMFSPTQPGRPHTGN |
| | | | VAIPQVTSVESKPLPPLAFKHTVGHIILSEHKGVKFNCSI |
| | | | SVPNIYQDTTISWWKDGKELLGAHHAITQFYPDDEVTA |
| | | | IIASFSITSVQRSDNGSYICKMKINNEEIVSDPIYIEVQGLP |
| | | | HFTKQPESMNVTRNTAFNLTCQAVGPPEPVNIFWVQNS |
| | | | SRVNEQPEKSPSVLTVPGLTEMAVFSCEAHNDKGLTVS |
| | | | KGVQINIKAIPSPPTEVSIRNSTAHSILISWVPGFDGYSPF |
| | | | RNCSIQVKEADPLSNGSVMIFNTSALPHLYQIKQLQALA |
| | | | NYSIGVSCMNEIGWSAVSPWILASTTEGAPSVAPLNVT |
| | | | VFLNESSDNVDIRWMKPPTKQQDGELVGYRISHVWQS |
| | | | AGISKELLEEVGQNGSRARISVQVHNATCTVRIAAVTR |
| | | | GGVGPFSDPVKIFIPAHGWVDYAPSSTPAPGNADPVLIIF |
| | | | GCFCGFILIGLILYISLAIRKRVQETKFGNAFTEEDSELVV |
| | | | NYIAKKSFCRRAIELTLHSLGVSEELQNKLEDVVIDRNL |
| | | | LILGKILGEGEFGSVMEGNLKQEDGTSLKVAVKTMKLD |
| | | | NSSQREIEEFLSEAACMKDFSHPNVIRLLGVCIEMSSQGI |
| | | | PKPMVILPFMKYGDLHTYLLYSRLETGPKHIPLQTLLKF |
| | | | MVDIALGMEYLSNRNFLHRDLAARNCMLRDDMTVCV |
| | | | ADFGLSKKIYSGDYYRQGRIAKMPVKWIAIESLADRVY |
| | | | TSKSDVWAFGVTMWEIATRGMTPYPGVQNHEMYDYL |
| | | | LHGHRLKQPEDCLDELYEIMYSCWRTDPLDRPTFSVLR |
| | | | LQLEKLLESLPDVRNQADVIYVNTQLLESSEGLAQGSTL |
| | | | APLDLNIDPDSIIASCTPRAAISVVTAEVHDSKPHEGRYI |
| | | | LNGGSEEWEDLTSAPSAAVTAEKNSVLPGERLVRNGVS |
| | | | WSHSSMLPLGSSLPDELLFADDSSEGSEVLM (SEQ ID |
| | | | NO: 76) |

TABLE 5
Exemplary Nucleotide Sequences for Cytokine Receptors

| Intracellular | | | |
|---|---|---|---|
| Signaling | Intracellular | | |
| Output | Domain From: | GeneID | Nucleotide Sequence |
| IL-10 | IL-10Ra | 3587 | ATGTTGCCTTGCCTCGTCGTGCTGCTTGCCGCTTTGCTG |
| | | | AGCCTCCGCCTGGGGTCCGACGCCCATGGTACTGAGCT |
| | | | GCCTTCTCCCCCTTCCGTGTGGTTCGAGGCCGAGTTTTT |
| | | | CCATCACATCCTCCACTGGACCCCCATCCCCAACCAGA |
| | | | GTGAATCTACATGCTACGAAGTGGCCCTTCTGCGCTAC |
| | | | GGCATTGAGAGCTGGAACTCTATCTCTAACTGCAGCCA |
| | | | GACACTGTCTTATGACCTGACGGCTGTGACACTGGACC |
| | | | TCTATCATTCCAATGGGTATCGCGCTCGCGTGAGGGCC |
| | | | GTGGACGGGAGCCGCCACAGCAACTGGACTGTTACCA |
| | | | ATACCCGCTTCTCCGTGGATGAGGTGACACTTACAGTG |
| | | | GGTAGTGTCAATCTGGAAATTCACAACGGCTTCATTCT |
| | | | GGGCAAAATCCAGCTGCCCCGCCCTAAGATGGCTCCG |
| | | | GCGAATGACACCTACGAGTCCATCTTTTCCCACTTTAG |
| | | | AGAGTACGAGATCGCTATTCGCAAAGTGCCCGGTAAC |
| | | | TTCACCTTCACCCACAAGAAAGTAAAGCATGAGAATT |
| | | | TTTCTCTCCTTACAAGCGGCGAAGTGGGAGAATTCTGC |
| | | | GTGCAGGTGAAACCGTCCGTGGCTTCTCGTTCAAACAA |
| | | | GGGGATGTGGAGTAAGGAGGAATGCATTTCCCTGACT |
| | | | CGCCAGTATTTCACTGTTACCAACGTCATTATCTTTTTC |
| | | | GCGTTTGTGTTGCTCCTGTCTGGTGCGCTTGCATACTGT |
| | | | CTGGCCCTCCAGCTGTATGTGAGACGCAGGAAAAAGC |
| | | | TGCCCAGTGTACTCCTGTTTAAAAAGCCCTCCCCTTTT |
| | | | ATCTTCATTTCCCAGAGGCCTTCCCCCGAGACACAGGA |
| | | | TACGATTCACCCCCTGGATGAAGAGGCCTTCCTGAAG |
| | | | GTGTCACCAGAGCTGAAGAACCTCGACCTCCACGGTA |
| | | | GCACCGACTCCGGCTTTGGCTCCACAAAACCCTCCCTG |
| | | | CAAACCGAAGAGCCCCAGTTCCTCCTGCCTGACCCGC |
| | | | ACCCGCAGGCTGACCGCACCCTGGGAAACCGCGAGCC |
| | | | GCCTGTGCTGGGAGATTCCTGCAGCTCCGGCAGCTCTA |
| | | | ACAGCACCGACTCTGGTATCTGTCTGCAGGAGCCCTCC |
| | | | CTGAGCCCTTCAACAGGCCCGACTTGGGAGCAACAGG |
| | | | TCGGGTCCAACTCTCGTGGACAAGACGATTCTGGCATC |
| | | | GACCTGGTCCAGAACTCTGAGGGTCGTGCTGGAGATA |
| | | | CACAGGGTGGCAGCGCTCTTGGCCACCATTCTCCCCCG |
| | | | GAGCCCGAGGTGCCTGGCGAAGAGGATCCCGCAGCCG |
| | | | TTGCATTCCAGGGATACCTGCGGCAGACTAGGTGCGC |
| | | | CGAAGAGAAGGCTACCAAGACCGGCTGTCTGGAGGAA |
| | | | GAGTCTCCCCTGACCGATGGTCTTGGCCCTAAATTCGG |
| | | | TCGCTGTCTCGTGGACGAGGCCGGTCTCCATCCCCCTG |
| | | | CACTGGCCAAGGGCTACCTGAAGCAGGACCCGCTGGA |
| | | | GATGACTCTGGCTAGCAGTGGCGCACCTACTGGTCAAT |
| | | | GGAATCAACCTACGGAGGAATGGTCCCTGTTGGCTCTC |
| | | | TCCTCATGCTCCGATCTCGGTATCTCCGATTGGAGTTT |
| | | | CGCCCACGATCTGGCCCCTCTGGGGTGCGTGGCCGCAC |
| | | | CAGGGGGCCTGTTGGGCAGCTTTAACTCTGATCTGGTG |
| | | | ACCCTGCCATTGATCAGCTCCCTTCAGAGCTCTGAG |
| | | | (SEQ ID NO: 77) |
| IL-4/IL-13 | IL4Ra | 3566 | ATGGGATGGTTGTGCAGCGGCCTTCTGTTTCCCGTCTC |
| | | | TTGTCTGGTACTGCTCCAGGTGGCTTCCAGCGGCAACA |
| | | | TGAAGGTCCTGCAGGAGCCCACCTGTGTTTCTGATTAT |
| | | | ATGTCTATCTCTACTTGCGAGTGGAAGATGAACGGCCC |
| | | | CACGAACTGTTCAACCGAACTGCGCTTGCTCTACCAGC |
| | | | TTGTGTTTTTGCTGTCCGAGGCGCATACTTGTATCCCG |
| | | | GAAAATAACGGGGGCGCGGGCTGTGTCTGCCATTTGC |
| | | | TGATGGATGACGTAGTGAGTGCCGATAACTATACTCTG |
| | | | GACCTGTGGGCTGGCCAGCAACTCCTGTGGAAGGGTA |
| | | | GTTTCAAACCGTCCGAACATGTTAAGCCCCGCGCTCCT |
| | | | GGCAACCTCACAGTGCACACCAATGTGTCTGACACAC |
| | | | TGCTCCTGACTTGGTCCAACCCTTATCCACCCGATAAT |
| | | | TACCTGTATAACCACCTCACGTATGCTGTAAACATCTG |
| | | | GTCTGAGAACGACCCCGCGGACTTCCGGATTTATAAC |
| | | | GTGACCTACCTCGAACCCTCCCTGCGTATCGCGGCCTC |
| | | | AACACTGAAGAGCGGTATCTCCTACCGCGCGCGTGTC |
| | | | AGGGCGTGGGCCCAATGTTACAACACTACCTGGTCTG |
| | | | AATGGTCTCCCTCAACAAAATGGCACAATTCTTACCGC |
| | | | GAACCCTTCGAGCAGCACCTGTTGCTGGGGGTGAGTG |
| | | | TTTCCTGCATCGTAATCCTGGCCGTGTGTCTTCTCTGTT |
| | | | ACGTGAGCATTACCAAGATTAAAAAGGAATGGTGGGA |
| | | | CCAGATCCCCAACCCTGCGAGGAGCCGCCTGGTGGCG |
| | | | ATCATTATCCAAGATGCCCAAGGCTCCCAGTGGGAGA |
| | | | AGCGCTCACGTGGTCAAGAGCCTGCTAAGTGCCCACA |
| | | | CTGGAAGAACTGCTTGACCAAATTGCTGCCTTGCTTCC |
| | | | TGGAACACAACATGAAGCGGGATGAGGACCCCCATAA |
| | | | AGCAGCTAAGGAAATGCCCTTTCAAGGCAGTGGCAAG |
| | | | TCCGCCTGGTGCCCTGTAGAGATCTCTAAGACCGTCCT |
| | | | GTGGCCTGAGTCTATTAGCGTAGTCCGCTGTGTGGAGC |
| | | | TGTTCGAGGCACCCGTTGAATGTGAAGAGGAAGAGGA |
| | | | AGTTGAAGAGGAAAAGGGCTCATTCTGCGCGAGTCCA |
| | | | GAGAGTAGCCGCGATGACTTCCAGGAAGGGCGCGAGG |
| | | | GTATTGTGGCTCGCCTGACGGAAAGTCTGTTCCTGGAT |
| | | | CTCCTGGGTGAGGAAAATGGGGGTTTCTGCCAACAGG |
| | | | ACATGGGAGAATCTTGCTTGCTGCCGCCCTCCGGCTCA |
| | | | ACCTCCGCTCACATGCCATGGGATGAATTTCCATCCGC |
| | | | GGGTCCTAAGGAGGCCCCACCTTGGGGGAAGGAGCAG |
| | | | CCCCTGCACCTGGAGCCCTCACCACCGGCTAGCCCAAC |
| | | | ACAGTCACCCGACAATCTGACTTGTACAGAGACTCCG |
| | | | CTGGTGATCGCCGGCAACCCAGCCTATCGGTCTTTCTC |
| | | | CAACTCTCTCTCTCAGAGCCCTTGTCCTCGCGAACTCG |
| | | | GCCCTGACCCCCTCCTGGCTCGCCACCTGGAGGAAGTG |
| | | | GAACCAGAGATGCCCTGCGTGCCCCAACTGTCTGAGC |
| | | | CCACGACCGTCCCACAGCCCGAACCGGAGACGTGGGA |
| | | | GCAGATCCTGCGGCGCAACGTGTTGCAGCACGGCGCC |
| | | | GCTGCCGCTCCCGTGAGCGCCCCTACCAGTGGCTATCA |
| | | | GGAGTTCGTCCACGCCGTTGAACAAGGCGGGACTCAA |
| | | | GCATCAGCAGTAGTGGGCCTCGGTCCCCCAGGCGAGG |
| | | | CTGGCTACAAGGCGTTCAGCTCCTTGCTTGCCTCATCT |
| | | | GCCGTGTCTCCAGAGAAGTGCGGTTTCGGGGCCTCCTC |
| | | | AGGGGAAGAGGGCTATAAGCCCTTCCAGGATCTCATC |
| | | | CCCGGCTGCCCTGGCGACCCTGCACCTGTCCCGGTACC |
| | | | ACTGTTCACCTTCGGCCTTGACAGAGAGCCCCCACGCT |
| | | | CCCCACAGTCCTCACACCTGCCGTCAAGCAGTCCTGAA |
| | | | CACCTGGGCTTGGAGCCCGGTGAGAAGGTTGAGGACA |
| | | | TGCCAAAGCCCCCGCTCCCGCAAGAGCAGGCTACCGA |
| | | | TCCCCTGGTAGACAGCCTGGGAAGCGGCATCGTGTATT |
| | | | CCGCCCTGACATGCCACTTGTGTGGACACCTCAAGCAG |
| | | | TGCCATGGTCAAGAGGATGGCGGTCAGACACCCGTGA |
| | | | TGGCTTCTCCGTGTTGCGGATGTTGCTGTGGCGACCGC |
| | | | TCTTCACCCCCAACCACACCGCTGCGCGCGCCGGATCC |
| | | | GAGTCCTGGCGGAGTCCCTCTGGAGGCTTCCCTGTGCC |
| | | | CCGCGTCTCTGGCCCCTTCCGGAATTTCTGAGAAAAGT |
| | | | AAATCTTCCAGCTCCTTTCACCCGGCGCCCGGTAATGC |
| | | | GCAGAGTTCAAGCCAGACACCTAAGATCGTGAACTTC |
| | | | GTAAGTGTCGGCCCCACATACATGCGCGTGTCT (SEQ |
| | | | ID NO: 78) |
| IL7/TSLP | ILRa | 3575 | ATGACCATCCTGGGCACTACCTTCGGCATGGTCTTTAG |
| | | | CCTTCTGCAGGTCGTGTCCGGCGAATCTGGATATGCCC |
| | | | AGAACGGTGACCTCGAGGACGCAGAACTGGATGACTA |
| | | | CAGCTTCTCCTGTTACTCTCAGCTGGAAGTAAACGGTT |
| | | | CCCAGCACTCCCTGACCTGCGCGTTTGAGGACCCAGAC |
| | | | GTCAACATCACCAACCTGGAGTTCGAGATCTGTGGCG |
| | | | CCCTGGTGGAGGTGAAGTGCCTGAACTTCCGTAAGCTC |
| | | | CAGGAGATCTACTTCATCGAAACCAAAAAGTTCCTGCT |
| | | | TATCGGAAAGAGTAACATTTGCGTCAAGGTGGGGGAG |
| | | | AAGTCACTCACTTGCAAAAAGATCGACCTGACCACGA |
| | | | TCGTGAAGCCAGAGGCTCCATTCGACCTGTCCGTCGTT |
| | | | TACAGGGAGGGCGCCAACGACTTTGTTGTGACCTTTAA |
| | | | CACCTCCCATCTGCAGAAAAAGTACGTCAAAGTGCTC |
| | | | ATGCATGACGTGGCTTATCGCCAAGAAAAGGATGAGA |
| | | | ACAAGTGGACACATGTGAACCTGAGTTCCACGAAGCT |
| | | | TACACTTCTGCAGCGCAAACTGCAGCCTGCCGCTATGT |
| | | | ATGAGATTAAGGTGCGTTCCATCCCGGACCATTATTTC |
| | | | AAGGGTTTCTGGAGTGAGTGGAGCCCCTCCTACTATTT |
| | | | CAGGACGCCGGAGATCAACAATAGCAGTGGGGAAATG |
| | | | GACCCCATCCTCCTGACCATCTCAATCTTGTCATTCTTT |
| | | | AGTGTGGCACTTTTGGTGATCCTTGCCTGCGTCCTGTG |
| | | | GAAAAAGCGCATCAAGCCGATCGTCTGGCCTAGTCTG |
| | | | CCAGACCACAAAAAGACTCTCGAGCATTTGTGCAAAA |
| | | | AGCCCCGCAAGAACCTGAACGTGAGTTTCAATCCAGA |
| | | | ATCCTTTCTCGATTGCCAGATCCACAGGGTGGATGACA |
| | | | TTCAGGCCCGCGACGAAGTCGAGGGATTTCTCCAGGA |
| | | | CACTTTTCCCCAGCAACTGGAAGAGTCAGAGAAGCAG |
| | | | CGCCTGGGTGGCGACGTGCAGTCCCCAAACTGCCCCA |
| | | | GTGAGGACGTTGTGATCACACCCGAGTCTTTCGGCAG |
| | | | GGACAGTAGCCTGACCTGCCTGGCCGGAAACGTGTCC |
| | | | GCTTGTGACGCTCCGATCCTCTCTAGCTCCCGGAGCCT |
| | | | GGACTGTCGCGAGTCCGGCAAGAACGGTCCTCACGTG |
| | | | TATCAGGACCTGCTCCTGAGCCTGGGTACTACGAACA |
| | | | GTACACTGCCCCCTCCCTTCTCCCTGCAGTCCGGCATC |
| | | | CTGACCCTGAATCCAGTGGCTCAGGGGCAGCCAATTCT |
| | | | GACCTCCCTGGGCTCAAATCAGGAGGAAGCTTACGTG |
| | | | ACAATGTCCAGCTTCTATCAGAATCAG (SEQ ID NO: |
| | | | 79) |
| IL9 | IL9Ra | 3581 | ATGGGGCTGGGTCGCTGCATCTGGGAGGGTTGGACTC |
| | | | TGGAGTCAGAAGCATTGAGGCGCGACATGGGCACCTG |
| | | | GCTGCTTGCCTGTATCTGTATCTGTACCTGTGTCTGTCT |
| | | | GGGCGTGTCCGTGACAGGGGAAGGCCAGGGGCCTCGG |
| | | | TCCCGTACTTTCACCTGTCTGACAAATAACATCCTGCG |
| | | | CATCGATTGCCATTGGTCAGCCCCTGAATTGGGGCAGG |
| | | | GGTCCAGTCCGTGGCTTCTGTTTACCTCCAATCAGGCT |
| | | | CCGGGTGGCACGCACAAGTGCATCCTCCGCGGCAGTG |
| | | | AGTGTACCGTGGTCCTTCCCCCAGAGGCAGTCCTGGTG |
| | | | CCGTCCGATAACTTCACAATTACCTTCCATCACTGTAT |
| | | | GAGCGGCCGTGAGCAAGTGTCACTGGTCGATCCCGAA |
| | | | TATCTGCCACGGCGCCATGTGAAGCTGGACCCTCCCTC |
| | | | CGATCTCCAGTCCAACATCTCTTCCGGCCACTGTATCT |
| | | | TGACATGGAGCATCTCCCCTGCTCTGGAGCCTATGACG |
| | | | ACCCTGTTGTCCTATGAACTGGCCTTCAAAAAGCAGGA |
| | | | AGAGGCTTGGGAGCAGGCGCAACACCGTGACCACATC |
| | | | GTGGGAGTGACCTGGCTTATTCTCGAGGCCTTCGAGCT |
| | | | GGACCCGGGTTTCATCCACGAAGCCCGGCTGCGTGTA |
| | | | CAAATGGCTACCCTGGAGGACGATGTTGTAGAGGAAG |
| | | | AGAGGTATACAGGGCAGTGGTCAGAGTGGTCTCAGCC |
| | | | TGTGTGTTTTCAGGCGCCTCAGCGGCAGGGACCCCTGA |
| | | | TCCCACCTTGGGGCTGGCCTGGCAATACCCTGGTCGCT |
| | | | GTGTCAATTTTTTTGCTGCTTACCGGCCCTACTTACTTG |
| | | | CTCTTTAAGCTGAGCCCCCGTGTTAAACGCATTTTCTA |
| | | | CCAGAACGTGCCATCTCCCGCTATGTTTTTCCAGCCCC |
| | | | TCTACAGCGTGCACAACGGCAACTTTCAAACCTGGAT |
| | | | GGGCGCCCACGGCGCTGGCGTGCTTCTGTCCCAGGACT |
| | | | GTGCCGGGACCCCACAGGGTGCTTTGGAACCTTGTGTC |
| | | | CAGGAGGCGACAGCCTTGCTGACCTGTGGCCCTGCCA |
| | | | GACCCTGGAAATCTGTTGCCCTGGAGGAAGAGCAGGA |
| | | | GGGGCCTGGGACTCGGCTGCCTGGGAACCTGTCTAGC |
| | | | GAGGATGTGCTGCCCGCCGGCTGCACCGAGTGGCGCG |
| | | | TCCAGACACTGGCGTACCTGCCCCAGGAAGATTGGGC |
| | | | CCCTACTTCTCTGACTCGCCCTGCTCCCCCAGATTCCG |
| | | | AGGGCTCCCGCTCTTCCAGCAGTTCCAGCTCTTCCAAT |
| | | | AACAATAACTATTGTGCCCTCGGCTGTTATGGCGGTTG |
| | | | GCATCTGTCAGCGCTGCCCGGGAACACCCAAAGCTCC |
| | | | GGGCCTATTCCTGCTCTCGCTTGCGGACTCTCCTGTGA |
| | | | TCATCAGGGATTGGAAACGCAGCAAGGAGTTGCTTGG |
| | | | GTCCTGGCTGGTCATTGCCAGCGCCCCGGTTTGCATGA |
| | | | AGACCTGCAGGGCATGTTGCTGCCTTCCGTGCTGTCTA |
| | | | AGGCGAGAAGCTGGACATTC (SEQ ID NO: 80) |
| IL21 | IL21Ra | 50615 | ATGCCCAGGGGATGGGCCGCGCCGCTCCTGCTCCTGCT |
| | | | CCTGCAGGGCGGATGGGGCTGTCCTGACCTGGTCTGTT |
| | | | ACACCGACTACTTGCAGACCGTGATCTGCATCCTCGAG |
| | | | ATGTGGAACCTGCATCCATCCACCCTTACTCTGACTTG |
| | | | GCAGGACCAGTACGAAGAGTTGAAGGATGAGGCAACC |
| | | | AGCTGTAGTTTGCACCGTTCCGCCCACAACGCCACGCA |
| | | | TGCTACATATACTTGCCATATGGACGTGTTTCACTTCA |
| | | | TGGCCGATGACATCTTTTCCGTGAATATCACAGATCAG |
| | | | TCCGGTAACTATTCCCAGGAGTGCGGGTCCTTCTTGCT |
| | | | GGCTGAGTCCATCAAGCCTGCCCCACCCTTCAACGTGA |
| | | | CCGTTACATTCTCTGGTCAGTACAACATCTCCTGGCGC |
| | | | AGCGATTACGAGGACCCTGCGTTTTACATGCTCAAGG |
| | | | GTAAGCTCCAGTACGAGCTGCAGTATCGGAACCGTGG |
| | | | TGACCCCTGGGCCGTCAGCCCCCGTAGAAAGTTGATC |
| | | | AGCGTGGACTCCCGCAGCGTGAGCTTGCTGCCCCTGG |
| | | | AGTTCCGCAAGGATTCTTCCTATGAGCTGCAAGTCCGC |
| | | | GCCGGCCCAATGCCAGGTAGTTCCTATCAAGGCACAT |
| | | | GGAGCGAATGGAGCGACCCAGTCATTTTCCAGACACA |
| | | | GTCTGAAGAGCTGAAAGAGGGTTGGAATCCGCACCTG |
| | | | TTGCTCCTTCTGCTCCTGGTGATCGTGTTTATCCCAGCT |
| | | | TTCTGGTCTCTCAAGACCCATCCCCTGTGGAGACTGTG |
| | | | GAAGAAAATCTGGGCCGTCCCTAGCCCGGAGCGCTTT |
| | | | TTCATGCCCCTGTATAAGGGTTGTTCTGGTGACTTCAA |
| | | | AAAGTGGGTAGGAGCGCCTTTTACTGGCTCTTCCCTGG |
| | | | AGCTTGGCCCTTGGAGTCCAGAGGTGCCCTCTACACTG |
| | | | GAGGTGTATTCTTGCCACCCACCTCGCTCTCCAGCCAA |
| | | | GCGCCTTCAACTGACCGAACTGCAGGAGCCAGCCGAA |
| | | | CTCGTGGAATCCGACGGAGTCCCAAAGCCGAGTTTCT |
| | | | GGCCAACCGCTCAGAATTCCGGTGGGAGCGCTTACTC |
| | | | CGAAGAGAGAGACCGCCCATATGGACTGGTATCCATT |
| | | | GACACGGTGACCGTGCTTGATGCCGAGGGACCCTGTA |
| | | | CATGGCCTTGCTCTTGTGAGGATGACGGGTACCCTGCT |
| | | | CTCGACCTGGATGCCGGTCTGGAACCCTCTCCCGGTTT |
| | | | GGAGGATCCATTGCTGGACGCGGGCACAACCGTTCTC |
| | | | TCCTGCGGCTGTGTGTCCGCTGGCTCCCCCGGCTTGGG |
| | | | TGGCCCCCTCGGAAGCCTGCTCGATCGCCTGAAGCCTC |
| | | | CGCTCGCGGATGGTGAGGATTGGGCCGGGGGCCTGCC |
| | | | ATGGGGAGGCAGGAGCCCTGGAGGCGTGTCCGAGTCC |
| | | | GAGGCCGGAAGTCCACTTGCAGGGCTGGACATGGACA |
| | | | CCTTTGACTCCGGCTTTGTGGGTTCCGACTGCTCTTCCC |
| | | | CCGTGGAGTGTGATTTTACTTCTCCAGGCGACGAAGGG |
| | | | CCTCCCCGTTCTTACTTGAGACAGTGGGTCGTGATCCC |
| | | | ACCGCCCCTGTCCAGCCCTGGCCCTCAGGCCTCC (SEQ |
| | | | ID NO: 81) |
| IL2/IL15 | IL2Rb | 3560 | ATGGCCGCACCAGCTCTTTCCTGGCGCCTCCCTCTGCT |
| | | | CATTCTTCTGTTGCCACTGGCTACATCCTGGGCCAGTG |
| | | | CTGCAGTGAACGGTACCAGCCAGTTTACCTGTTTCTAC |
| | | | AACTCCCGCGCTAACATTTCTTGTGTGTGGAGTCAGGA |
| | | | CGGGGCGCTGCAGGATACCTCCTGCCAGGTTCACGCCT |
| | | | GGCCCGATCGCCGTCGCTGGAACCAGACTTGCGAATT |
| | | | GCTGCCGGTGAGCCAAGCCTCCTGGGCCTGTAATCTGA |
| | | | TTCTCGGTGCCCCCGATTCTCAGAAGCTGACTACCGTC |
| | | | GATATTGTTACCTTGCGTGTTCTGTGTAGAGAAGGAGT |
| | | | GCGCTGGCGCGTGATGGCTATTCAGGACTTTAAACCCT |
| | | | TCGAGAACCTGAGGCTCATGGCACCCATCTCACTTCAG |
| | | | GTAGTGCATGTGGAGACTCACCGCTGCAATATTTCCTG |
| | | | GGAGATCTCCCAGGCGTCCCATTACTTCGAGCGTCACC |
| | | | TCGAATTTGAGGCTCGTACCCTGAGCCCGGGACACAC |
| | | | ATGGGAAGAGGCTCCACTCCTGACTTTGAAGCAGAAA |
| | | | CAGGAGTGGATCTGCCTGGAGACCCTGACCCCCGACA |
| | | | CACAGTATGAGTTCCAGGTGCGCGTGAAGCCCCTTCA |
| | | | GGGCGAGTTCACTACCTGGTCTCCGTGGAGCCAGCCCT |
| | | | TGGCATTCCGTACGAAGCCAGCGGCTCTGGGAAAGGA |
| | | | TACGATTCCTTGGCTGGGTCATCTGCTCGTGGGCCTCT |
| | | | CCGGGGCCTTCGGTTTTATTATCCTGGTCTACTTGCTG |
| | | | ATTAACTGCCGGAACACTGGACCTTGGCTGAAGAAAG |
| | | | TGCTCAAATGTAATACACCCGACCCAAGCAAGTTCTTT |
| | | | TCTCAGCTGTCTTCCGAGCATGGAGGCGATGTGCAGA |
| | | | AGTGGTTGTCCTCTCCCTTCCCTTCTAGCTCCTTCAGCC |
| | | | CGGGCGGACTGGCGCCGGAGATCTCTCCCCTGGAGGT |
| | | | CCTGGAGCGCGATAAAGTGACCCAACTGCTCCTGCAA |
| | | | CAGGACAAGGTGCCCGAGCCTGCTAGCCTCTCTTCAA |
| | | | ACCACTCCCTGACCTCCTGTTTTACGAACCAGGGCTAT |
| | | | TTCTTTTTCCATTTGCCCGATGCTTTGGAGATCGAAGC |
| | | | CTGTCAGGTGTATTTCACCTATGACCCGTACTCTGAAG |
| | | | AGGATCCCGATGAAGGCGTGGCCGGTGCTCCAACCGG |
| | | | AAGCAGTCCCCAGCCACTGCAGCCTCTGTCCGGTGAA |
| | | | GATGACGCTTACTGTACCTTCCCCAGTAGGGATGACTT |
| | | | GCTCCTGTTCAGTCCCAGCCTCCTGGGTGGACCCAGCC |
| | | | CTCCCTCTACCGCGCCTGGAGGGTCCGGCGCCGGTGA |
| | | | AGAGCGCATGCCGCCTTCCCTGCAAGAGAGAGTGCCA |
| | | | CGCGACTGGGATCCCCAGCCACTCGGTCCCCCAACTCC |
| | | | TGGCGTGCCCGACCTGGTCGATTTCCAGCCACCTCCGG |
| | | | AGTTGGTCCTCCGTGAAGCTGGCGAAGAGGTGCCAGA |
| | | | CGCTGGTCCTAGGGAGGGGGTCAGCTTTCCATGGAGC |
| | | | CGCCCACCCGGTCAGGGGGAGTTCCGCGCTCTTAACG |
| | | | CTCGGCTGCCCTTGAACACAGATGCTTACCTTTCCCTC |
| | | | CAGGAGCTGCAAGGACAAGATCCGACCCACCTGGTG |
| | | | (SEQ ID NO: 82) |
| IL6/IL11 | gp130 | 3572 | ATGCTGACCTTGCAGACTTGGCTGGTGCAGGCTCTGTT |
| | | | TATCTTCTTGACAACCGAATCTACTGGAGAACTCCTGG |
| | | | ATCCCTGTGGCTACATCTCTCCCGAAAGTCCCGTTGTG |
| | | | CAGCTTCATTCCAATTTCACTGCGGTGTGTGTGCTTAA |
| | | | GGAGAAGTGTATGGATTACTTCCACGTGAACGCGAAC |
| | | | TACATCGTATGGAAGACAAACCACTTCACCATTCCTAA |
| | | | GGAGCAGTACACGATTATCAATAGAACGGCATCCAGC |
| | | | GTGACATTCACCGACATCGCGTCCCTGAATATTCAGCT |
| | | | GACCTGTAATATCCTCACCTTCGGCCAGCTTGAACAGA |
| | | | ACGTGTACGGCATCACTATTATCAGTGGGCTGCCCCCT |
| | | | GAAAAGCCCAAAAATCTGTCTTGCATTGTGAACGAGG |
| | | | GGAAAAAGATGCGCTGCGAATGGGACGGTGGCCGTGA |
| | | | GACACATCTCGAGACGAATTTTACCTTGAAGTCCGAGT |
| | | | GGGCCACCCACAAGTTCGCCGACTGTAAGGCTAAACG |
| | | | TGACACCCCCACCTCCTGCACAGTGGATTACAGCACCG |
| | | | TCTACTTCGTGAACATCGAAGTCTGGGTGGAGGCGGA |
| | | | GAACGCGCTGGGTAAGGTGACCTCTGACCACATCAAC |
| | | | TTTGACCCCGTGTATAAGGTGAAGCCCAACCCACCTCA |
| | | | CAATCTTTCCGTTATCAACTCCGAAGAGCTCTCTTCCA |
| | | | TTCTTAAGTTGACCTGGACAAACCCTTCCATCAAGAGC |
| | | | GTGATCATTCTGAAATACAACATCCAGTATCGCACAA |
| | | | AGGACGCCTCCACTTGGAGCCAGATCCCTCCGGAGGA |
| | | | CACCGCGAGTACCCGCAGCTCTTTCACAGTCCAGGACC |
| | | | TTAAACCGTTTACTGAGTATGTGTTCCGCATCAGGTGC |
| | | | ATGAAGGAGGACGGCAAGGGATACTGGAGCGACTGGT |
| | | | CCGAAGAGGCTAGCGGTATTACATACGAGGACCGTCC |
| | | | TTCTAAGGCCCCTTCTTTTTGGTACAAGATCGACCCCA |
| | | | GCCATACACAGGGTTATCGGACCGTCCAGTTGGTGTG |
| | | | GAAGACCCTGCCTCCCTTCGAAGCCAACGGTAAGATC |
| | | | CTGGACTACGAGGTTACCCTGACCCGGTGGAAGTCTC |
| | | | ACCTGCAGAACTACACCGTCAATGCAACAAAGCTGAC |
| | | | AGTGAACCTGACCAACGACCGCTATCTCGCAACTCTG |
| | | | ACAGTGCGCAACCTGGTCGGTAAAAGTGATGCTGCCG |
| | | | TACTGACTATCCCAGCGTGTGACTTTCAGGCAACCCAT |
| | | | CCAGTCATGGACCTGAAGGCCTTCCCAAAGGATAACA |
| | | | TGCTGTGGGTTGAGTGGACCACTCCGAGGGAAAGTGT |
| | | | TAAAAAGTACATCCTGGAGTGGTGCGTGCTGTCTGAC |
| | | | AAGGCCCCCTGCATCACCGACTGGCAGCAAGAAGATG |
| | | | GCACTGTACACCGTACTTACCTCCGCGGGAACCTTGCC |
| | | | GAATCAAAATGTTACCTGATCACTGTGACCCCCGTGTA |
| | | | CGCAGACGGTCCCGGTAGTCCCGAATCAATTAAGGCC |
| | | | TACCTCAAGCAGGCTCCACCGTCCAAGGGCCCCACTGT |
| | | | CCGTACCAAAAAGGTGGGTAAAAACGAAGCTGTGCTG |
| | | | GAGTGGGACCAGCTGCCGGTGGACGTGCAGAACGGCT |
| | | | TTATTCGTAACTACACAATTTTCTATCGCACGATTATC |
| | | | GGAAATGAAACAGCGGTGAACGTGGACTCCAGCCACA |
| | | | CCGAGTACACCCTCAGCTCCTTGACCAGTGACACGCTC |
| | | | TACATGGTTCGGATGGCTGCCTATACCGATGAAGGAG |
| | | | GTAAAGATGGACCTGAGTTCACCTTTACGACCCCCAA |
| | | | GTTTGCACAGGGCGAAATTGAAGCAATCGTCGTGCCA |
| | | | GTCTGCCTCGCCTTCCTCCTGACCACTCTCCTGGGGGT |
| | | | CCTGTTTTGTTTCAACAAGCGGGATCTGATCAAAAAGC |
| | | | ATATCTGGCCAAATGTTCCCGACCCTAGTAAGTCCCAT |
| | | | ATTGCTCAGTGGTCTCCCCACACCCCTCCCAGGCACAA |
| | | | TTTTAACTCCAAGGACCAAATGTACTCAGACGGCAACT |
| | | | TCACCGATGTATCAGTTGTGGAGATCGAGGCTAACGA |
| | | | CAAGAAACCTTTCCCCGAGGACCTCAAATCCCTGGATC |
| | | | TGTTTAAGAAAGAGAAGATCAACACAGAAGGTCATTC |
| | | | TTCCGGAATCGGTGGCTCCAGCTGCATGAGCTCAAGCC |
| | | | GCCCAAGCATCAGCTCCAGCGACGAGAACGAGAGCAG |
| | | | TCAGAACACATCCAGCACCGTTCAGTATTCCACTGTAG |
| | | | TCCATTCTGGATACCGTCACCAGGTCCCCAGCGTGCAA |
| | | | GTGTTCTCCAGGAGCGAGTCCACCCAGCCCCTGCTCGA |
| | | | TTCAGAAGAGCGGCCAGAGGATCTGCAGCTGGTCGAC |
| | | | CACGTGGACGGGGGTGATGGTATCCTGCCAAGGCAAC |
| | | | AGTACTTCAAGCAAAATTGCTCCCAACACGAATCTTCC |
| | | | CCGGACATCTCCCATTTCGAGAGATCTAAGCAGGTCTC |
| | | | CAGCGTCAACGAAGAGGACTTTGTGAGGCTGAAACAA |
| | | | CAGATTTCCGATCATATCAGCCAGAGCTGCGGTTCCGG |
| | | | ACAGATGAAAATGTTCCAGGAGGTGTCTGCTGCCGAC |
| | | | GCGTTTGGCCCCGGGACCGAAGGACAAGTCGAGAGGT |
| | | | TCGAGACCGTTGGTATGGAGGCCGCTACAGACGAGGG |
| | | | CATGCCGAAAAGCTACCTGCCCCAGACCGTGCGCCAA |
| | | | GGTGGCTACATGCCTCAA (SEQ ID NO: 83) |
| IL6 | IL6Ra | 3570 | ATGCTGGCGGTGGGCTGCGCTTTGCTGGCGGCCCTGCT |
| | | | CGCTGCCCCTGGAGCGGCCCTGGCTCCGCGTCGCTGCC |
| | | | CCGCTCAGGAGGTCGCGCGTGGAGTGTTGACTTCTCTG |
| | | | CCTGGCGATAGCGTGACTTTGACTTGCCCTGGCGTAGA |
| | | | GCCTGAGGATAACGCTACCGTGCATTGGGTACTGAGA |
| | | | AAGCCCGCTGCCGGCTCCCATCCTTCCCGCTGGGCAGG |
| | | | CATGGGGAGGCGCCTGCTCCTGCGGTCAGTTCAATTGC |
| | | | ACGACTCTGGTAATTACAGCTGCTACCGGGCCGGACG |
| | | | CCCAGCCGGCACCGTCCATCTGCTCGTCGATGTGCCGC |
| | | | CAGAGGAACCACAGCTCTCCTGTTTTAGGAAGTCTCCA |
| | | | CTTTCCAACGTTGTGTGTGAGTGGGGCCCCCGGAGCAC |
| | | | CCCTAGCTTGACAACTAAGGCCGTGCTGCTCGTGCGCA |
| | | | AATTTCAGAACTCCCCCGCCGAAGACTTTCAGGAGCC |
| | | | GTGCCAGTACAGCCAGGAATCCCAGAAGTTCAGTTGC |
| | | | CAGCTGGCCGTTCCAGAGGGTGACTCCAGCTTCTACAT |
| | | | TGTGAGTATGTGCGTGGCGAGTTCAGTCGGCTCAAAGT |
| | | | TTTCAAAGACCCAGACTTTTCAGGGATGTGGTATTCTC |
| | | | CAGCCGGATCCTCCCGCCAACATCACTGTCACCGCCGT |
| | | | GGCACGGAACCCTCGCTGGCTGAGCGTCACATGGCAG |
| | | | GACCCCCACTCCTGGAACAGTAGCTTTTACCGTCTCCG |
| | | | CTTCGAGCTCCGTTATAGAGCTGAGCGCTCCAAGACCT |
| | | | TCACCACATGGATGGTAAAGGACCTGCAGCATCACTG |
| | | | TGTCATCCACGACGCCTGGTCTGGACTGCGTCATGTTG |
| | | | TGCAGCTCAGGGCCCAGGAAGAGTTCGGGCAGGGGGA |
| | | | GTGGTCCGAATGGTCTCCGGAGGCCATGGGCACACCA |
| | | | TGGACTGAGAGCAGATCCCCACCCGCTGAAAACGAGG |
| | | | TTTCTACCCCAATGCAGGCGCTGACCACTAACAAAGA |
| | | | CGATGACAATATCCTGTTCCGCGATAGTGCCAACGCG |
| | | | ACCTCCCTGCCCGTGCAGGACTCCTCTTCCGTGCCACT |
| | | | CCCAACGTTTCTGGTGGCCGGAGGCTCTTTGGCCTTCG |
| | | | GGACTCTTCTGTGCATCGCGATTGTGTTGCGCTTCAAA |
| | | | AAGACATGGAAGTTGCGCGCGCTCAAGGAGGGCAAGA |
| | | | CTTCTATGCATCCACCTTATTCTCTGGGTCAATTGGTGC |
| | | | CTGAGAGGCCTAGACCCACCCCCGTGCTGGTCCCCTTG |
| | | | ATTTCCCCTCCCGTCTCCCCAAGCTCACTGGGAAGCGA |
| | | | CAACACTAGTTCCCACAACAGGCCAGATGCACGGGAT |
| | | | CCCCGTTCACCGTACGACATCTCAAACACCGACTATTT |
| | | | TTTCCCTAGA (SEQ ID NO: 84) |
| IL11 | IL11Ra | 3590 | ATGAGCTCCTCTTGCTCCGGTCTCTCCCGCGTGCTCGT |
| | | | GGCCGTTGCTACAGCTCTGGTGTCTGCCAGCTCTCCTT |
| | | | GTCCTCAGGCGTGGGGGCCCCCTGGCGTGCAGTACGG |
| | | | ACAGCCCGGCCGCTCTGTGAAGCTGTGTTGCCCCGGCG |
| | | | TGACAGCAGGCGATCCTGTGAGTTGGTTTAGAGACGG |
| | | | AGAGCCAAAACTCCTGCAGGGACCTGACTCCGGTCTG |
| | | | GGGCATGAGTTGGTCCTGGCCCAGGCCGATAGTACGG |
| | | | ACGAGGGGACCTACATCTGCCAGACTCTCGACGGCGC |
| | | | CCTGGGAGGCACGGTGACGCTGCAGCTGGGTTACCCT |
| | | | CCAGCTAGACCCGTGGTCTCCTGTCAGGCCGCTGACTA |
| | | | TGAGAATTTCAGTTGTACATGGAGCCCCAGTCAGATTT |
| | | | CAGGCTTGCCCACTCGCTACCTGACTTCCTACAGAAAA |
| | | | AAGACAGTCCTGGGAGCAGACAGCCAGCGCAGGTCAC |
| | | | CCAGTACCGGACCTTGGCCGTGCCCTCAGGACCCTTTG |
| | | | GGTGCTGCCCGTTGCGTTGTGCATGGCGCCGAGTTCTG |
| | | | GTCTCAGTATAGGATCAATGTAACCGAAGTGAACCCT |
| | | | CTGGGTGCGTCCACCCGGCTGCTCGACGTTTCTTTGCA |
| | | | GAGCATCCTGCGCCCCGACCCGCCCCAAGGACTTCGC |
| | | | GTGGAGTCTGTCCCCGGCTACCCTCGTCGGCTGCGCGC |
| | | | GAGTTGGACTTACCCGGCCAGCTGGCCCTGCCAACCCC |
| | | | ACTTTCTCCTGAAATTTCGTCTGCAGTATCGCCCCGCT |
| | | | CAGCACCCTGCCTGGTCTACTGTGGAACCCGCGGGACT |
| | | | GGAAGAGGTTATCACAGACGCCGTCGCTGGCCTGCCC |
| | | | CATGCTGTGCGCGTTTCAGCCCGCGACTTCCTGGACGC |
| | | | CGGGACCTGGAGCACCTGGTCCCCAGAGGCTTGGGGC |
| | | | ACTCCAAGCACCGGCACAATCCCTAAGGAGATCCCTG |
| | | | CTTGGGGCCAGCTGCACACTCAGCCAGAGGTGGAGCC |
| | | | TCAGGTTGACTCTCCCGCTCCCCCTCGCCCTAGCCTGC |
| | | | AGCCTCACCCCAGGCTCTTGGACCACCGCGATAGCGTC |
| | | | GAGCAGGTGGCAGTGCTGGCGTCTCTGGGGATCCTTTC |
| | | | CTTTCTCGGCCTGGTCGCCGGCGCTCTGGCACTGGGGC |
| | | | TCTGGCTCAGGCTCAGGCGTGGTGGCAAGGACGGCTC |
| | | | CCCTAAGCCTGGCTTCCTGGCCAGTGTGATCCCTGTGG |
| | | | ATCGCAGGCCCGGAGCGCCTAATTTG (SEQ ID NO: 85) |
| GCSF | GCSFR | 1441 | ATGGCCAGACTGGGCAACTGTTCCCTGACATGGGCCG |
| | | | CACTGATTATCCTGCTCCTGCCGGGCAGCCTGGAAGAG |
| | | | TGCGGCCACATCTCTGTGTCAGCCCCCATCGTGCACCT |
| | | | GGGTGACCCGATCACCGCTTCTTGCATTATCAAGCAGA |
| | | | ACTGTTCCCACCTGGACCCCGAACCTCAGATTTTGTGG |
| | | | CGCCTGGGCGCCGAATTGCAGCCAGGTGGCCGCCAAC |
| | | | AGCGCCTGTCCGATGGCACCCAGGAGAGCATCATTAC |
| | | | CCTTCCCCACCTTAATCATACACAGGCCTTCCTGTCTT |
| | | | GTTGCCTGAACTGGGGTAATTCCCTGCAAATCCTGGAT |
| | | | CAGGTGGAGCTGCGCGCCGGCTACCCGCCTGCAATCC |
| | | | CTCATAATCTGAGCTGTCTGATGAATTTGACTACCAGC |
| | | | AGTCTGATCTGTCAGTGGGAGCCGGGCCCGGAAACTC |
| | | | ATCTTCCGACTTCATTTACACTGAAATCCTTTAAAAGC |
| | | | CGCGGCAACTGTCAGACTCAGGGAGATTCCATCCTGG |
| | | | ATTGCGTCCCAAAGGACGGCCAGAGCCACTGCTGTAT |
| | | | TCCTCGCAAGCACCTGCTTCTGTACCAAAACATGGGTA |
| | | | TCTGGGTCCAGGCAGAGAACGCCCTGGGGACATCCAT |
| | | | GTCTCCCCAGCTGTGTCTGGACCCCATGGATGTTGTGA |
| | | | AATTGGAGCCCCCAATGCTGCGTACCATGGACCCTAG |
| | | | CCCCGAGGCTGCGCCACCCCAGGCCGGCTGTCTGCAG |
| | | | CTGTGTTGGGAGCCGTGGCAACCCGGTCTTCACATCAA |
| | | | CCAGAAGTGTGAACTCCGCCACAAGCCCCAGCGTGGA |
| | | | GAAGCAAGCTGGGCTCTGGTGGGCCCCCTCCCTCTGG |
| | | | AGGCCCTGCAGTACGAGCTGTGTGGGCTTCTGCCTGCG |
| | | | ACAGCTTATACCCTGCAGATCCGCTGTATCCGTTGGCC |
| | | | CCTGCCGGGCCACTGGTCCGACTGGTCCCCCTCCCTGG |
| | | | AGCTGAGGACTACCGAGCGTGCGCCTACTGTCCGCCT |
| | | | GGATACCTGGTGGCGTCAGCGCCAGCTCGACCCTCGC |
| | | | ACCGTCCAGCTGTTCTGGAAACCAGTGCCGCTGGAGG |
| | | | AAGATAGCGGACGCATCCAGGGGTATGTCGTGTCTTG |
| | | | GCGGCCCTCCGGACAGGCTGGGGCGATCCTGCCCCTCT |
| | | | GTAACACCACGGAGTTGAGTTGTACCTTCCATCTCCCT |
| | | | TCTGAGGCCCAGGAAGTGGCGTTGGTGGCCTACAACT |
| | | | CAGCAGGCACCAGTAGACCCACACCCGTGGTCTTTTCT |
| | | | GAGAGCCGGGGACCAGCTCTGACTCGGCTCCATGCGA |
| | | | TGGCACGGGATCCACATTCCCTGTGGGTGGGGTGGGA |
| | | | GCCCCCTAATCCTTGGCCGCAGGGCTACGTGATCGAAT |
| | | | GGGGGCTTGGACCCCCATCCGCATCCAATTCCAACAA |
| | | | GACCTGGCGGATGGAGCAGAATGGTAGAGCAACCGGG |
| | | | TTCCTCCTGAAGGAAAATATTAGGCCGTTCCAACTCTA |
| | | | CGAAATTATCGTCACCCCTCTGTACCAAGACACTATGG |
| | | | GTCCCAGCCAGCACGTGTATGCGTACTCCCAGGAGAT |
| | | | GGCCCCCTCACACGCCCCAGAACTTCACTTGAAGCAC |
| | | | ATCGGTAAGACATGGGCCCAGCTGGAGTGGGTACCAG |
| | | | AGCCGCCCGAGCTCGGTAAAAGCCCTCTGACACACTA |
| | | | CACTATCTTCTGGACCAACGCCCAGAACCAGTCCTTCT |
| | | | CTGCCATTCTGAACGCGTCTTCCCGCGGCTTTGTGCTG |
| | | | CACGGCCTCGAGCCCGCGAGCCTGTACCACATTCACCT |
| | | | CATGGCAGCTAGCCAAGCCGGCGCAACCAACTCCACC |
| | | | GTTCTGACCCTGATGACCCTGACCCCCGAGGGCTCAGA |
| | | | ACTGCACATTATCCTGGGCCTCTTTGGATTGCTGCTCTT |
| | | | GCTGACCTGTCTGTGCGGCACTGCATGGCTCTGCTGTT |
| | | | CCCCTAACCGCAAGAACCCACTGTGGCCTTCTGTGCCC |
| | | | GATCCAGCGCATTCCAGTCTGGGCTCCTGGGTGCCGAC |
| | | | CATCATGGAAGAGGACGCCTTTCAGCTCCCAGGTCTG |
| | | | GGGACGCCCCCGATTACAAAGCTGACTGTTCTGGAAG |
| | | | AGGACGAGAAAAAGCCTGTGCCATGGGAGTCCCACAA |
| | | | TTCCTCTGAAACTTGTGGTCTGCCTACCCTGGTGCAGA |
| | | | CCTACGTGCTGCAGGGGGACCCAAGAGCTGTTTCTACC |
| | | | CAGCCTCAGTCCCAGAGCGGCACATCTGATCAGGTGTT |
| | | | GTATGGCCAACTTCTCGGTAGCCCGACTAGCCCCGGCC |
| | | | CTGGACATTACTTGCGGTGTGACAGCACCCAGCCATTG |
| | | | CTGGCCGGCCTGACACCATCCCCAAAGTCCTATGAGA |
| | | | ACCTCTGGTTTCAGGCTTCTCCACTCGGCACCCTGGTC |
| | | | ACCCCGGCCCCCAGCCAGGAAGATGACTGTGTCTTTG |
| | | | GCCCCCTCCTGAACTTCCCCCTTCTGCAGGGAATCCGC |
| | | | GTCCACGGTATGGAGGCCTTGGGTTCCTTC (SEQ ID |
| | | | NO: 86) |
| IL3 | IL3Ra | 3563 | ATGGTGCTGCTTTGGTTGACCCTGCTCCTGATTGCCCT |
| | | | GCCATGCCTCCTGCAGACGAAGGAGGATCCCAACCCG |
| | | | CCCATCACCAACCTGAGAATGAAGGCGAAGGCTCAAC |
| | | | AGCTCACTTGGGACCTGAACCGTAACGTGACCGACAT |
| | | | CGAGTGCGTGAAAGATGCCGATTACTCCATGCCAGCA |
| | | | GTCAATAACTCTTATTGTCAGTTCGGTGCCATCAGCTT |
| | | | GTGTGAGGTGACTAATTACACCGTGCGCGTGGCGAAC |
| | | | CCACCCTTCAGTACCTGGATCCTGTTCCCCGAGAATAG |
| | | | CGGTAAGCCCTGGGCCGGCGCCGAGAACCTGACGTGC |
| | | | TGGATCCATGACGTGGACTTTCTGAGCTGTTCCTGGGC |
| | | | CGTGGGCCCAGGCGCCCCAGCCGATGTACAGTACGAC |
| | | | CTGTATCTGAACGTCGCCAATAGGCGCCAGCAATATG |
| | | | AGTGCCTTCACTATAAGACCGACGCACAAGGTACCCG |
| | | | TATTGGCTGTCGCTTTGATGACATTTCCAGACTGAGCT |
| | | | CCGGCTCCCAGAGCTCTCATATCCTGGTTCGGGGGCGC |
| | | | TCCGCTGCATTTGGTATCCCCTGTACCGATAAATTTGT |
| | | | TGTGTTCAGCCAGATTGAGATTCTGACTCCCCCTAACA |
| | | | TGACGGCGAAATGCAACAAGACACATTCATTTATGCA |
| | | | CTGGAAGATGCGGTCCCATTTTAACCGCAAATTTCGGT |
| | | | ATGAACTCCAGATTCAGAAGCGTATGCAGCCCGTCAT |
| | | | CACCGAGCAGGTGCGCGATCGCACCAGTTTCCAGCTTC |
| | | | TGAACCCGGGCACCTATACAGTGCAAATCAGAGCAAG |
| | | | AGAACGCGTATACGAATTCCTGTCTGCGTGGTCTACCC |
| | | | CTCAGCGTTTTGAGTGCGACCAAGAGGAAGGTGCCAA |
| | | | CACACGCGCTTGGCGCACCAGCCTCTTGATTGCCCTGG |
| | | | GCACACTTCTGGCCCTGGTGTGTGTGTTTGTGATCTGC |
| | | | CGCAGATACCTCGTGATGCAGCGTCTGTTCCCCCGTAT |
| | | | CCCTCACATGAAGGACCCGATCGGCGACTCCTTTCAGA |
| | | | ACGATAAGCTTGTAGTGTGGGAGGCCGGTAAGGCCGG |
| | | | TTTGGAAGAGTGCCTGGTGACCGAAGTCCAGGTTGTG |
| | | | CAAAAAACT (SEQ ID NO: 87) |
| IL5 | IL5Ra | 3568 | ATGATTATCGTGGCCCACGTGTTGCTGATCCTGCTCGG |
| | | | TGCGACTGAAATTCTTCAGGCCGATCTTCTCCCTGATG |
| | | | AGAAAATCTCACTTCTGCCGCCCGTAAACTTTACAATC |
| | | | AAGGTCACAGGCCTGGCTCAAGTACTTCTGCAATGGA |
| | | | AACCAAACCCCGACCAAGAACAGCGGAATGTGAACCT |
| | | | GGAGTATCAAGTGAAGATTAACGCCCCCAAGGAGGAC |
| | | | GATTACGAAACCCGCATCACAGAGTCCAAGTGCGTCA |
| | | | CCATCCTGCACAAGGGCTTTAGCGCGAGTGTCCGTACC |
| | | | ATCCTGCAGAACGACCATAGCCTCCTGGCCTCTAGTTG |
| | | | GGCCAGTGCTGAGCTGCACGCTCCCCCTGGATCCCCAG |
| | | | GTACCAGCATCGTGAACCTTACCTGCACTACCAATACC |
| | | | ACTGAGGACAACTATAGCAGGCTCCGCTCTTACCAGG |
| | | | TATCTCTGCACTGCACTTGGCTGGTGGGCACAGACGCG |
| | | | CCCGAGGACACCCAGTATTTTTTGTATTACCGTTACGG |
| | | | CTCTTGGACTGAAGAGTGCCAGGAGTACTCTAAGGAT |
| | | | ACTCTGGGCCGTAATATCGCATGCTGGTTCCCTCGCAC |
| | | | TTTTATCTTGTCCAAAGGGCGGGATTGGTTGGCCGTGC |
| | | | TGGTGAATGGCTCCAGCAAGCACTCCGCTATCCGCCCA |
| | | | TTCGATCAGCTGTTCGCTCTGCACGCGATCGACCAGAT |
| | | | CAATCCCCCGCTGAACGTGACCGCTGAGATCGAGGGC |
| | | | ACTCGGCTGTCTATCCAGTGGGAGAAACCGGTGAGTG |
| | | | CCTTCCCCATCCACTGTTTCGACTACGAGGTGAAGATT |
| | | | CACAACACCAGAAACGGCTATCTGCAGATCGAGAAGC |
| | | | TGATGACCAACGCCTTTATTTCCATTATCGATGACTTG |
| | | | TCCAAATACGACGTGCAGGTGCGCGCGGCTGTCAGCT |
| | | | CTATGTGCCGCGAGGCCGGGTTGTGGAGTGAGTGGAG |
| | | | CCAGCCCATCTACGTGGGCAACGACGAGCACAAGCCT |
| | | | CTTCGGGAATGGTTCGTGATCGTCATCATGGCCACCAT |
| | | | CTGTTTCATCTTGCTGATCCTCAGTCTGATCTGCAAGA |
| | | | TTTGCCATCTCTGGATCAAGCTCTTTCCCCCTATCCCCG |
| | | | CCCCCAAATCTAACATCAAGGATCTTTTTGTGACAACC |
| | | | AACTATGAGAAAGCTGGTTCCAGCGAAACCGAAATTG |
| | | | AGGTGATCTGCTACATTGAAAAACCTGGAGTGGAGAC |
| | | | GTTGGAAGACTCCGTTTTT (SEQ ID NO: 88) |
| GM-CSF | GMCSFRa | 1438 | ATGCTGCTTCTCGTGACCTCCCTGCTCCTGTGTGAGCT |
| | | | GCCGCACCCTGCCTTTCTCCTGATCCCAGAGAAGAGCG |
| | | | ACCTGCGGACCGTTGCCCCGGCCTCCTCTCTGAACGTA |
| | | | AGATTCGACAGCCGGACCATGAACCTCAGCTGGGATT |
| | | | GCCAGGAAAACACAACCTTCTCCAAGTGCTTCCTGACT |
| | | | GACAAAAAGAATCGCGTAGTGGAACCTCGCCTGTCCA |
| | | | ATAACGAGTGCTCCTGTACCTTCCGCGAGATCTGTCTG |
| | | | CACGAGGGTGTGACCTTTGAAGTCCACGTCAACACTA |
| | | | GCCAGAGAGGGTTCCAACAGAAGTTGCTGTACCCGAA |
| | | | CTCTGGACGGGAGGGCACCGCTGCCCAGAATTTCTCAT |
| | | | GCTTTATCTACAACGCTGACCTTATGAACTGCACCTGG |
| | | | GCACGTGGCCCCACCGCGCCCCGTGACGTCCAGTACTT |
| | | | CCTCTACATCCGCAATTCCAAGAGACGGCGTGAGATT |
| | | | AGGTGTCCATATTACATCCAGGACTCTGGCACCCATGT |
| | | | GGGGTGCCATCTGGATAACCTGAGCGGCCTTACCTCTC |
| | | | GCAACTACTTTCTCGTTAATGGCACGAGCAGAGAGAT |
| | | | CGGCATCCAGTTTTTCGATTCTCTTCTGGACACAAAGA |
| | | | AAATCGAAAGGTTCAACCCACCCTCTAACGTCACCGTT |
| | | | CGCTGTAACACTACCCATTGTTTGGTGAGGTGGAAACA |
| | | | GCCACGCACCTACCAGAAGCTGTCATATCTGGACTTCC |
| | | | AGTACCAGCTGGACGTCCATCGCAAAAATACACAGCC |
| | | | TGGCACCGAAAACCTGCTTATCAACGTGTCCGGCGATC |
| | | | TGGAGAACCGCTACAACTTCCCTAGTTCTGAGCCGCGC |
| | | | GCTAAACACAGTGTCAAAATCAGAGCTGCCGACGTGA |
| | | | GGATTCTCAACTGGAGTTCCTGGAGTGAAGCAATCGA |
| | | | GTTCGGCTCAGATGACGGTAACCTGGGCTCTGTGTATA |
| | | | TCTACGTCCTGCTTATCGTGGGTACACTGGTTTGTGGC |
| | | | ATTGTTCTGGGTTTCCTTTTCAAGAGGTTCCTGAGAAT |
| | | | CCAGAGGCTGTTTCCCCCAGTGCCGCAAATCAAAGAT |
| | | | AAGCTCAACGACAACCACGAGGTGGAAGATGAGATTA |
| | | | TCTGGGAGGAATTCACTCCTGAAGAGGGAAAAGGCTA |
| | | | TAGGGAGGAAGTTCTGACCGTGAAGGAGATTACT |
| | | | (SEQ ID NO: 89) |
| IL3/5/GM-CSF | CSF2Rb | 1439 | ATGGTCCTGGCACAGGGACTCCTTTCCATGGCCCTGCT |
| | | | TGCTCTTTGTTGGGAGCGCAGCTTGGCCGGCGCCGAAG |
| | | | AGACCATCCCTTTGCAGACCCTGCGCTGTTATAATGAC |
| | | | TACACTTCACACATCACATGTCGCTGGGCCGACACGCA |
| | | | GGACGCTCAACGCCTCGTGAACGTGACCCTCATTCGGC |
| | | | GCGTGAACGAGGACCTGCTTGAGCCTGTGTCATGTGAT |
| | | | CTTAGCGATGACATGCCCTGGTCCGCCTGTCCACACCC |
| | | | GCGGTGTGTACCGCGGCGCTGCGTCATCCCTTGTCAGT |
| | | | CCTTCGTCGTGACTGACGTGGACTACTTTTCCTTCCAA |
| | | | CCTGACAGACCCCTGGGTACTCGCCTGACGGTTACTCT |
| | | | GACCCAGCACGTGCAGCCACCCGAACCTCGCGACCTG |
| | | | CAGATCTCTACCGACCAAGACCACTTTCTTCTGACTTG |
| | | | GTCCGTGGCTCTTGGGAGCCCTCAAAGTCATTGGCTCA |
| | | | GCCCCGGTGACCTGGAGTTCGAGGTGGTTTACAAGCG |
| | | | TCTTCAGGATTCTTGGGAGGATGCGGCTATCCTGCTCT |
| | | | CCAACACCAGCCAGGCTACCCTTGGCCCCGAGCACCT |
| | | | GATGCCATCATCCACCTATGTGGCCCGCGTGAGGACG |
| | | | CGCCTGGCACCTGGCAGTCGTCTGTCTGGTAGACCCTC |
| | | | CAAGTGGAGTCCTGAGGTCTGTTGGGACAGCCAGCCT |
| | | | GGCGATGAAGCTCAGCCTCAGAACCTGGAGTGCTTTTT |
| | | | CGACGGCGCGGCTGTGCTGTCTTGTAGCTGGGAGGTG |
| | | | CGCAAAGAAGTTGCTAGCTCTGTGTCCTTCGGCCTTTT |
| | | | CTATAAACCCTCTCCTGACGCCGGTGAAGAGGAATGC |
| | | | TCTCCTGTGCTGCGTGAGGGCCTGGGCTCCCTGCATAC |
| | | | CCGTCACCATTGCCAGATTCCTGTACCGGACCCAGCCA |
| | | | CCCACGGCCAATATATTGTGAGTGTGCAGCCCCGTCGC |
| | | | GCCGAAAAACATATTAAGAGCTCCGTGAACATTCAGA |
| | | | TGGCTCCGCCTTCCCTGAATGTGACCAAGGACGGCGA |
| | | | CAGCTACAGCCTGCGCTGGGAGACCATGAAAATGCGT |
| | | | TACGAGCACATTGACCACACTTTTGAAATTCAATATAG |
| | | | GAAGGACACCGCAACCTGGAAGGACTCTAAGACTGAG |
| | | | ACTCTGCAGAACGCCCACTCAATGGCCCTGCCCGCGCT |
| | | | TGAGCCAAGCACCAGATACTGGGCTCGTGTGCGTGTG |
| | | | CGCACCAGTCGCACAGGTTACAACGGCATCTGGAGCG |
| | | | AATGGAGCGAGGCTCGTAGCTGGGACACCGAGTCCGT |
| | | | CCTGCCGATGTGGGTCCTGGCCCTGATCGTCATCTTCC |
| | | | TCACCATCGCCGTCTTGCTGGCCCTGCGTTTCTGTGGG |
| | | | ATTTACGGATATCGTCTGCGGAGAAAATGGGAGGAAA |
| | | | AGATCCCTAACCCGAGCAAATCCCACCTGTTCCAGAA |
| | | | CGGGAGCGCCGAGCTGTGGCCGCCCGGTTCTATGTCC |
| | | | GCTTTCACCTCAGGCAGCCCACCCCACCAGGGCCCATG |
| | | | GGGGTCTCGGTTCCCTGAGTTGGAGGGCGTCTTCCCCG |
| | | | TAGGGTTCGGCGATAGCGAGGTGAGTCCTCTGACAAT |
| | | | CGAGGATCCGAAACACGTGTGTGACCCTCCATCTGGC |
| | | | CCCGACACCACACCCGCGGCCTCCGACCTTCCTACAGA |
| | | | GCAGCCCCCTAGCCCCCAGCCCGGTCCTCCCGCTGCCT |
| | | | CCCATACACCCGAGAAGCAGGCTAGCTCTTTTGATTTC |
| | | | AACGGGCCTTACCTGGGACCACCCCATTCCAGGTCTCT |
| | | | GCCAGATATCCTGGGTCAGCCAGAACCACCGCAGGAG |
| | | | GGAGGGTCCCAGAAGTCTCCCCCGCCCGGATCCCTGG |
| | | | AGTACCTTTGTCTGCCGGCAGGAGGCCAGGTTCAGCTG |
| | | | GTGCCTCTGGCCCAGGCCATGGGGCCCGGGCAGGCAG |
| | | | TTGAAGTGGAGCGGCGTCCAAGTCAAGGTGCGGCCGG |
| | | | CAGTCCGTCTCTGGAGTCCGGAGGGGGCCCGGCCCCA |
| | | | CCGGCCCTGGGCCCCAGGGTCGGCGGTCAGGACCAGA |
| | | | AGGACTCCCCCGTAGCCATTCCCATGTCCAGCGGCGAT |
| | | | ACAGAGGATCCCGGTGTTGCCTCAGGATACGTTTCCAG |
| | | | CGCGGATCTCGTGTTTACCCCTAATTCCGGCGCTTCAA |
| | | | GCGTCTCTCTGGTGCCTTCTCTTGGTCTGCCGAGCGAC |
| | | | CAGACGCCCAGCCTGTGTCCCGGTCTGGCTTCTGGACC |
| | | | CCCTGGCGCCCCCGGCCCTGTCAAGAGCGGTTTTGAGG |
| | | | GCTACGTCGAGCTGCCTCCGATCGAAGGGCGCTCTCCC |
| | | | CGCTCTCCTCGCAATAACCCCGTGCCTCCCGAGGCGAA |
| | | | ATCACCTGTGCTGAACCCAGGTGAACGCCCTGCCGAT |
| | | | GTCTCCCCTACCTCACCCCAGCCGGAGGGACTCCTGGT |
| | | | GCTTCAACAGGTGGGTGACTATTGTTTCCTTCCAGGGC |
| | | | TGGGCCCTGGACCCCTGTCTCTGCGTTCAAAGCCTTCC |
| | | | AGCCCTGGCCCTGGCCCTGAAATTAAGAACCTGGATC |
| | | | AGGCATTTCAGGTCAAAAAGCCTCCCGGCCAGGCCGT |
| | | | TCCTCAGGTGCCGGTTATTCAACTGTTCAAGGCCCTGA |
| | | | AGCAACAGGATTACCTGTCTCTGCCCCCATGGGAGGT |
| | | | GAACAAGCCCGGCGAGGTTTGT (SEQ ID NO: 90) |
| LIF | LIFRb | 3977 | ATGATGGATATTTATGTGTGCCTCAAACGCCCCTCATG |
| | | | GATGGTCGATAACAAGAGGATGAGGACCGCTAGTAAC |
| | | | TTTCAGTGGCTCCTGAGCACCTTCATCCTGCTCTACCTC |
| | | | ATGAACCAGGTGAACTCCCAGAAAAAGGGAGCGCCTC |
| | | | ACGACCTGAAGTGTGTGACAAACAATCTGCAGGTCTG |
| | | | GAATTGCAGCTGGAAGGCTCCTTCCGGCACTGGCCGT |
| | | | GGGACGGACTACGAGGTTTGCATCGAAAATAGAAGCC |
| | | | GCTCTTGCTATCAGTTGGAGAAGACCAGCATCAAGAT |
| | | | CCCGGCCCTGTCCCATGGCGACTACGAGATCACTATCA |
| | | | ACAGCCTGCATGATTTCGGGAGCTCCACGTCCAAGTTT |
| | | | ACTCTCAATGAGCAGAACGTGTCCCTTATCCCTGATAC |
| | | | TCCTGAGATTTTGAATTTGAGCGCTGACTTCTCAACCT |
| | | | CTACGCTGTATTTGAAGTGGAACGATCGCGGCTCCGTG |
| | | | TTCCCGCATCGCAGTAACGTGATCTGGGAGATCAAGG |
| | | | TCCTGCGTAAGGAAAGTATGGAGCTGGTGAAGCTGGT |
| | | | GACACATAATACTACCTTGAACGGTAAGGACACCTTG |
| | | | CATCACTGGTCTTGGGCGAGTGACATGCCTCTCGAATG |
| | | | CGCCATCCACTTTGTGGAGATCAGGTGCTACATTGACA |
| | | | ACCTGCATTTCTCCGGTCTGGAAGAGTGGAGTGACTGG |
| | | | TCCCCCGTGAAGAATATCTCCTGGATTCCAGATTCCCA |
| | | | GACAAAGGTGTTTCCTCAGGACAAGGTCATCCTGGTG |
| | | | GGCTCCGATATCACCTTTTGTTGCGTGTCTCAAGAGAA |
| | | | AGTGCTCTCCGCTTTGATTGGACATACAAACTGCCCTC |
| | | | TGATTCACTTGGATGGCGAGAACGTGGCCATCAAGAT |
| | | | CCGCAACATCTCCGTGAGCGCTAGTTCAGGAACCAAC |
| | | | GTGGTCTTCACAACCGAGGACAATATCTTTGGCACTGT |
| | | | GATCTTTGCGGGCTATCCACCCGACACACCCCAACAGC |
| | | | TGAATTGTGAGACTCATGATCTGAAGGAAATTATCTGT |
| | | | TCCTGGAACCCTGGACGCGTCACCGCCCTGGTCGGCCC |
| | | | TCGCGCGACCTCATACACTCTGGTAGAATCTTTTTCTG |
| | | | GAAAGTACGTGCGCTTGAAGCGTGCCGAGGCCCCAAC |
| | | | TAACGAATCATACCAGCTCCTGTTCCAGATGCTTCCCA |
| | | | ATCAGGAAATTTATAACTTCACACTCAATGCCCACAAC |
| | | | CCTCTCGGCAGATCCCAGAGCACCATCCTGGTGAACAT |
| | | | TACAGAGAAAGTGTACCCTCACACACCCACAAGTTTC |
| | | | AAGGTGAAGGACATCAACTCTACGGCGGTAAAACTGT |
| | | | CTTGGCATCTGCCTGGAAACTTCGCTAAGATCAACTTC |
| | | | CTGTGTGAGATCGAAATCAAAAAGAGCAATAGCGTGC |
| | | | AGGAGCAGAGGAACGTCACCATCAAAGGTGTGGAGA |
| | | | ACTCCTCTTACTTGGTCGCATTGGATAAGTTGAATCCT |
| | | | TACACACTGTACACATTCAGGATTAGATGTTCCACGGA |
| | | | GACTTTCTGGAAGTGGTCTAAATGGAGCAACAAAAAG |
| | | | CAACACCTCACAACCGAGGCCAGCCCGTCTAAGGGCC |
| | | | CTGACACGTGGCGGGAGTGGTCCTCAGACGGCAAGAA |
| | | | CCTCATCATTTATTGGAAGCCGCTGCCTATTAACGAGG |
| | | | CAAATGGGAAGATTTTGTCTTACAATGTCAGCTGCTCA |
| | | | TCCGACGAAGAGACCCAGTCCCTCAGCGAGATCCCCG |
| | | | ACCCACAGCATAAGGCCGAGATTCGTTTGGACAAGAA |
| | | | CGACTACATTATCTCAGTCGTGGCTAAGAACTCCGTGG |
| | | | GGTCCAGCCCGCCCTCTAAGATCGCCAGCATGGAGAT |
| | | | CCCCAACGATGACTTGAAGATCGAGCAAGTGGTTGGC |
| | | | ATGGGCAAGGGCATCTTGCTCACCTGGCATTATGATCC |
| | | | AAACATGACATGCGATTACGTCATTAAGTGGTGCAATT |
| | | | CTAGCCGGTCCGAGCCTTGTTTGATGGACTGGAGGAA |
| | | | AGTGCCCAGTAACTCAACCGAGACAGTGATCGAGTCT |
| | | | GACGAATTCCGCCCAGGGATTCGTTACAACTTTTTCCT |
| | | | TTATGGTTGCCGCAATCAGGGCTATCAACTTCTGCGCA |
| | | | GTATGATTGGATACATTGAAGAGCTCGCCCCCATCGTC |
| | | | GCACCTAATTTCACCGTCGAGGACACAAGTGCAGACA |
| | | | GCATCCTCGTCAAGTGGGAGGACATTCCGGTGGAAGA |
| | | | GCTCCGGGGATTTCTCAGGGGTTATCTCTTCTATTTCG |
| | | | GCAAGGGCGAGCGCGACACTTCTAAGATGCGCGTACT |
| | | | CGAGAGCGGCCGTAGCGATATCAAGGTTAAGAATATT |
| | | | ACGGACATCTCACAAAAAACCCTGAGGATCGCCGACT |
| | | | TGCAAGGCAAGACTTCTTACCATCTGGTGCTTCGCGCT |
| | | | TACACCGACGGCGGTGTGGGCCCTGAGAAGTCTATGT |
| | | | ACGTGGTTACGAAGGAGAACTCCGTGGGCCTGATTAT |
| | | | CGCTATCCTGATTCCTGTGGCTGTGGCAGTGATCGTGG |
| | | | GTGTGGTCACAAGCATTCTGTGCTACCGGAAGAGAGA |
| | | | GTGGATCAAGGAGACATTTTATCCCGATATTCCGAACC |
| | | | CCGAGAACTGTAAGGCCCTGCAGTTTCAGAAGTCCGT |
| | | | ATGTGAGGGCAGTTCTGCTCTGAAGACCTTGGAAATG |
| | | | AACCCATGTACGCCCAATAACGTTGAAGTACTGGAGA |
| | | | CTCGCTCCGCCTTCCCAAAGATCGAGGATACCGAGATT |
| | | | ATCTCCCCAGTGGCCGAACGTCCCGAGGACCGCTCTG |
| | | | ATGCAGAACCGGAGAACCACGTCGTGGTTTCATACTG |
| | | | TCCCCCGATTATCGAAGAGGAAATCCCGAACCCCGCT |
| | | | GCCGACGAGGCCGGTGGCACAGCCCAAGTGATCTACA |
| | | | TCGACGTGCAGAGCATGTATCAGCCCCAGGCCAAACC |
| | | | CGAGGAAGAGCAGGAAAATGACCCCGTCGGCGGTGCG |
| | | | GGCTATAAGCCCCAGATGCACCTGCCCATCAACTCCAC |
| | | | AGTTGAAGACATCGCCGCTGAAGAGGACTTGGACAAG |
| | | | ACCGCAGGCTATCGTCCTCAGGCAAACGTGAACACCT |
| | | | GGAACCTTGTAAGCCCTGACAGTCCTCGCAGCATCGA |
| | | | CTCAAACTCCGAGATCGTTTCTTTCGGCTCTCCGTGTTC |
| | | | CATTAACTCCCGGCAGTTTCTGATTCCCCCTAAGGACG |
| | | | AAGATTCCCCCAAATCTAACGGTGGCGGATGGTCCTTC |
| | | | ACCAACTTTTTCCAGAACAAACCAAACGAT (SEQ ID |
| | | | NO: 91) |
| IL31 | IL31Ra | 133396 | ATGATGTGGACGTGGGCCTTGTGGATGCTGCCCAGCCT |
| | | | GTGTAAGTTCAGCCTCGCTGCCTTGCCAGCCAAGCCTG |
| | | | AAAACATCAGTTGTGTCTATTACTATCGTAAGAACCTG |
| | | | ACTTGCACTTGGAGCCCGGGAAAAGAGACCAGCTATA |
| | | | CCCAGTACACCGTGAAGCGCACATATGCTTTTGGAGA |
| | | | GAAGCATGATAATTGTACAACTAACTCTAGCACCTCTG |
| | | | AAAACAGGGCCAGTTGTTCTTTTTTCCTGCCCCGTATT |
| | | | ACCATCCCTGACAACTACACCATTGAAGTGGAAGCCG |
| | | | AGAATGGCGACGGCGTGATCAAGAGTCACATGACCTA |
| | | | TTGGAGACTTGAAAATATCGCCAAGACTGAACCGCCT |
| | | | AAGATTTTTCGTGTGAAGCCCGTGCTGGGGATCAAGC |
| | | | GCATGATTCAGATTGAATGGATCAAGCCTGAGCTGGC |
| | | | CCCAGTGAGCTCTGACCTGAAATACACTCTGCGCTTCA |
| | | | GAACTGTTAACTCTACCTCCTGGATGGAAGTTAACTTT |
| | | | GCCAAGAACCGTAAGGACAAGAACCAGACATATAACC |
| | | | TGACGGGCCTGCAGCCTTTTACAGAGTACGTCATCGCC |
| | | | CTGCGTTGCGCTGTGAAAGAGAGCAAGTTCTGGTCTG |
| | | | ACTGGTCCCAGGAGAAGATGGGAATGACTGAAGAGGA |
| | | | AGCCCCTTGCGGTCTGGAGTTGTGGCGCGTGCTGAAAC |
| | | | CCGCTGAGGCCGACGGCAGACGCCCCGTGCGTTTGCT |
| | | | GTGGAAAAAGGCGAGGGGAGCTCCTGTTCTGGAAAAA |
| | | | ACACTGGGATACAACATCTGGTATTACCCTGAATCTAA |
| | | | CACTAATCTGACCGAGACCATGAACACAACCAATCAA |
| | | | CAGCTGGAGCTGCATCTCGGAGGCGAGTCCTTCTGGGT |
| | | | GTCCATGATCAGTTACAATAGCCTCGGCAAGTCCCCCG |
| | | | TGGCCACGCTGCGGATCCCCGCTATCCAGGAGAAGAG |
| | | | CTTTCAGTGCATCGAGGTCATGCAGGCCTGCGTGGCGG |
| | | | AAGACCAGCTGGTGGTTAAATGGCAGTCCTCTGCCCTG |
| | | | GACGTCAACACCTGGATGATCGAGTGGTTTCCTGACGT |
| | | | CGACTCCGAACCCACGACACTCTCTTGGGAGTCCGTTT |
| | | | CCCAAGCCACAAACTGGACCATCCAGCAAGACAAACT |
| | | | GAAACCCTTCTGGTGTTATAACATTTCCGTGTATCCTA |
| | | | TGCTGCACGACAAGGTAGGGGAGCCATACTCCATCCA |
| | | | AGCCTACGCCAAAGAGGGGGTTCCGTCAGAAGGCCCC |
| | | | GAGACTAAAGTGGAAAATATCGGCGTGAAAACAGTCA |
| | | | CCATTACCTGGAAGGAGATCCCCAAGTCCGAGCGCAA |
| | | | AGGCATCATTTGCAATTATACAATTTTCTATCAGGCCG |
| | | | AAGGGGGAAAGGGCTTCTCCAAGACCGTGAACAGTAG |
| | | | CATTCTGCAGTACGGACTGGAATCCCTGAAACGCAAG |
| | | | ACCTCCTATATTGTGCAGGTGATGGCCTCCACCAGCGC |
| | | | CGGCGGTACAAACGGCACCTCAATCAATTTCAAGACT |
| | | | CTGTCCTTCTCCGTATTTGAGATTATCTTGATTACTTCC |
| | | | CTGATCGGGGGTGGCCTTCTGATTCTCATTATCCTCAC |
| | | | CGTAGCATACGGCCTGAAAAAGCCTAATAAGCTGACC |
| | | | CACCTCTGTTGGCCAACCGTGCCAAACCCCGCTGAGA |
| | | | GTAGCATCGCTACCTGGCACGGCGATGACTTCAAGGA |
| | | | CAAACTGAACTTGAAGGAATCTGATGACTCTGTGAAC |
| | | | ACTGAGGACAGAATCCTCAAGCCCTGCTCCACACCGA |
| | | | GTGATAAGCTCGTCATTGACAAACTCGTGGTTAACTTC |
| | | | GGGAACGTCCTGCAGGAGATTTTTACCGACGAAGCCA |
| | | | GGACCGGCCAGGAGAATAACCTGGGAGGTGAGAAGA |
| | | | ACGGGTATGTGACATGCCCGTTCAGACCCGATTGTCCG |
| | | | CTGGGGAAGAGTTTTGAAGAGCTTCCTGTCTCTCCGGA |
| | | | AATCCCACCCCGCAAGAGCCAGTATCTCCGTTCCCGCA |
| | | | TGCCAGAGGGCACCCGCCCTGAGGCAAAGGAGCAGCT |
| | | | GCTCTTCTCTGGCCAGAGTCTGGTGCCCGACCACCTGT |
| | | | GTGAAGAGGGAGCCCCCAATCCATACTTGAAGAACTC |
| | | | TGTGACAGCCAGAGAGTTCCTGGTTAGCGAAAAGCTG |
| | | | CCTGAGCACACCAAGGGTGAGGTA (SEQ ID NO: 92) |
| CNTF/CT-1 | CNTFR | 1271 | ATGGCCGCGCCAGTCCCATGGGCTTGTTGCGCTGTACT |
| | | | GGCGGCCGCTGCAGCCGTTGTGTACGCTCAGCGCCACT |
| | | | CACCCCAGGAGGCTCCCCACGTACAATATGAACGCCT |
| | | | CGGTTCAGACGTCACCTTGCCGTGTGGCACAGCAAACT |
| | | | GGGATGCGGCCGTGACTTGGCGCGTGAACGGTACCGA |
| | | | TCTGGCCCCCGACTTGCTGAACGGATCTCAGCTCGTGC |
| | | | TGCATGGCTTGGAGCTGGGGCATAGCGGCCTGTACGC |
| | | | TTGCTTTCATAGAGATAGCTGGCACCTGCGCCACCAGG |
| | | | TGCTCCTGCACGTGGGACTGCCACCCCGCGAACCTGTG |
| | | | CTGAGCTGTCGCAGCAACACTTACCCCAAGGGATTCTA |
| | | | CTGCAGCTGGCATCTGCCCACACCCACCTACATCCCCA |
| | | | ACACATTCAATGTGACGGTCCTCCACGGCTCAAAGATC |
| | | | ATGGTGTGTGAAAAGGACCCTGCCCTGAAGAACCGGT |
| | | | GTCACATCCGTTACATGCATTTGTTTTCTACCATTAAGT |
| | | | ACAAGGTTTCTATCAGCGTGTCCAACGCATTGGGTCAC |
| | | | AACGCAACAGCTATTACTTTCGACGAGTTCACAATCGT |
| | | | GAAACCGGATCCTCCGGAGAACGTCGTTGCCCGGCCC |
| | | | GTCCCCAGTAACCCGAGGCGCCTCGAGGTGACCTGGC |
| | | | AGACACCATCCACCTGGCCCGATCCCGAGTCTTTCCCT |
| | | | TTGAAGTTCTTTCTCCGCTATAGACCTTTGATCCTGGAT |
| | | | CAGTGGCAGCACGTCGAGCTGAGTGACGGTACAGCTC |
| | | | ACACTATTACAGATGCATACGCCGGCAAGGAGTACAT |
| | | | TATCCAGGTAGCGGCTAAAGACAACGAAATCGGAACT |
| | | | TGGTCCGACTGGTCAGTCGCAGCCCACGCCACCCCGTG |
| | | | GACCGAGGAACCTAGGCACCTCACTACCGAGGCCCAA |
| | | | GCCGCTGAGACAACTACGTCAACCACTTCTTCCCTGGC |
| | | | GCCCCCTCCAACCACAAAGATCTGTGACCCCGGGGAG |
| | | | TTGGGCAGTGGCGGTGGCCCCAGCGCCCCATTCCTGGT |
| | | | TTCCGTGCCGATTACTCTCGCATTGGCTGCCGCTGCGG |
| | | | CCACCGCTTCTAGCCTGCTGATC (SEQ ID NO: 93) |
| IL27 | IL27Ra | 9466 | ATGCGGGGCGGGCGCGGCGCTCCGTTTTGGCTGTGGC |
| | | | CCCTGCCCAAGCTTGCTTTGCTGCCGTTGCTGTGGGTA |
| | | | CTGTTCCAGCGGACCCGCCCGCAGGGGTCAGCAGGCC |
| | | | CTCTCCAGTGCTACGGCGTGGGCCCATTGGGCGACCTC |
| | | | AACTGCTCTTGGGAGCCCCTCGGGGACCTGGGCGCCC |
| | | | CGAGTGAGCTTCATTTGCAGAGCCAAAAGTACCGTAG |
| | | | CAACAAAACTCAGACAGTCGCTGTCGCCGCGGGCAGG |
| | | | AGCTGGGTAGCGATCCCCAGGGAGCAGCTGACGATGT |
| | | | CCGATAAGCTTCTGGTGTGGGGCACAAAGGCAGGGCA |
| | | | GCCGCTGTGGCCTCCAGTCTTTGTCAACCTCGAGACTC |
| | | | AGATGAAACCCAACGCTCCACGTCTCGGTCCCGACGT |
| | | | CGACTTTTCTGAAGACGATCCCCTGGAGGCCACGGTTC |
| | | | ACTGGGCTCCCCCAACATGGCCAAGTCATAAGGTTCTG |
| | | | ATCTGCCAGTTCCACTATAGGCGCTGTCAGGAGGCCGC |
| | | | TTGGACCCTGCTCGAGCCTGAGCTCAAGACTATCCCAC |
| | | | TGACCCCGGTGGAGATCCAGGACCTGGAGCTGGCAAC |
| | | | CGGATATAAGGTATACGGCCGCTGCCGCATGGAGAAG |
| | | | GAAGAGGATCTGTGGGGCGAGTGGAGCCCCATCCTGT |
| | | | CTTTCCAAACTCCTCCAAGTGCTCCCAAGGACGTCTGG |
| | | | GTGTCCGGCAACCTGTGTGGAACACCTGGTGGAGAGG |
| | | | AACCTCTCTTGCTCTGGAAAGCTCCCGGACCTTGTGTC |
| | | | CAGGTGAGTTACAAGGTCTGGTTTTGGGTCGGAGGTC |
| | | | GCGAGCTGTCCCCTGAGGGCATCACTTGCTGTTGCAGC |
| | | | TTGATTCCATCTGGCGCTGAGTGGGCGCGTGTGTCAGC |
| | | | TGTGAACGCGACAAGTTGGGAGCCCCTGACAAATTTG |
| | | | TCCTTGGTCTGCCTGGACTCCGCGTCCGCTCCCCGCAG |
| | | | CGTGGCAGTCTCCTCTATCGCGGGGTCCACCGAGTTGC |
| | | | TGGTGACCTGGCAGCCGGGCCCTGGCGAGCCTCTTGA |
| | | | GCATGTTGTGGATTGGGCGCGTGATGGAGACCCGCTG |
| | | | GAGAAGCTCAACTGGGTCCGCCTGCCTCCCGGTAACCT |
| | | | GTCCGCCCTGCTCCCAGGCAATTTTACCGTTGGCGTGC |
| | | | CATATCGGATTACAGTGACCGCCGTGAGTGCTAGCGG |
| | | | GCTGGCGTCCGCGAGCTCCGTATGGGGTTTTAGGGAA |
| | | | GAGTTGGCTCCCCTGGTCGGCCCTACTCTGTGGAGGCT |
| | | | GCAGGACGCCCCTCCCGGCACACCCGCCATCGCCTGG |
| | | | GGAGAGGTGCCTAGGCATCAGCTGAGAGGCCACCTGA |
| | | | CTCACTATACACTCTGCGCTCAGTCAGGGACATCCCCC |
| | | | TCTGTATGTATGAATGTTTCTGGCAATACCCAATCCGT |
| | | | CACACTCCCAGACCTGCCTTGGGGCCCTTGCGAGCTTT |
| | | | GGGTGACAGCATCCACTATTGCTGGCCAGGGCCCACC |
| | | | TGGCCCTATTCTGCGTCTGCACCTGCCAGACAACACTC |
| | | | TGCGCTGGAAGGTCCTGCCAGGCATTCTGTTCCTCTGG |
| | | | GGGCTGTTCCTGTTGGGCTGTGGCCTCAGCCTGGCCAC |
| | | | ATCAGGCAGGTGCTATCACCTGAGACACAAGGTTCTG |
| | | | CCACGCTGGGTATGGGAGAAGGTGCCTGATCCAGCTA |
| | | | ACAGCTCTAGCGGCCAGCCGCATATGGAGCAGGTGCC |
| | | | TGAGGCACAGCCGCTGGGCGACCTTCCCATCCTGGAG |
| | | | GTCGAAGAGATGGAACCCCCTCCAGTTATGGAGTCTTC |
| | | | CCAGCCGGCCCAGGCTACCGCCCCACTTGATTCCGGCT |
| | | | ACGAGAAGCACTTCCTGCCAACCCCGGAAGAGCTCGG |
| | | | CCTGCTTGGTCCCCCTCGCCCGCAGGTGCTGGCT (SEQ |
| | | | ID NO: 94) |
| EPO | EPOR | 2057 | ATGGACCACCTGGGCGCGTCTTTGTGGCCACAGGTGG |
| | | | GCTCCTTGTGTTTGCTGTTGGCTGGAGCAGCCTGGGCT |
| | | | CCCCCTCCCAACTTGCCCGACCCCAAGTTTGAGAGCAA |
| | | | GGCTGCGCTCCTGGCTGCCAGGGGTCCTGAAGAGCTTC |
| | | | TCTGTTTCACTGAAAGATTGGAGGACCTGGTGTGTTTT |
| | | | TGGGAGGAAGCTGCCTCTGCAGGCGTCGGCCCGGGAA |
| | | | ATTACTCCTTTTCCTATCAGCTCGAGGACGAGCCGTGG |
| | | | AAGCTGTGTCGGCTTCACCAGGCCCCAACCGCTCGGG |
| | | | GTGCTGTCCGCTTCTGGTGTTCTCTCCCCACCGCAGAC |
| | | | ACTAGCTCCTTCGTTCCGCTGGAGCTGAGAGTCACTGC |
| | | | GGCCAGCGGCGCCCCTAGATACCATCGCGTGATTCAC |
| | | | ATCAATGAGGTGGTTCTTCTCGACGCCCCCGTGGGCCT |
| | | | TGTCGCCAGACTCGCTGATGAGAGCGGGCACGTTGTG |
| | | | CTCAGGTGGCTGCCACCGCCTGAGACCCCCATGACCTC |
| | | | CCACATCCGCTATGAAGTGGACGTTTCAGCCGGCAAT |
| | | | GGTGCCGGGTCAGTCCAGAGGGTGGAGATCTTGGAAG |
| | | | GCCGTACCGAATGCGTGCTGAGTAATTTGCGCGGGCG |
| | | | GACCCGCTACACCTTCGCTGTGCGTGCTCGTATGGCCG |
| | | | AGCCCAGCTTCGGCGGATTCTGGTCCGCCTGGTCTGAG |
| | | | CCTGTGAGTCTTCTGACGCCATCTGACCTGGATCCTTT |
| | | | GATTCTGACCCTGAGCTTGATCCTCGTCGTGATTCTGG |
| | | | TCCTGCTCACGGTCCTGGCTCTGCTTTCCCATCGTAGG |
| | | | GCACTGAAGCAGAAGATCTGGCCTGGCATCCCATCTC |
| | | | CGGAATCCGAGTTCGAAGGTCTGTTTACCACACACAA |
| | | | AGGTAACTTTCAGTTGTGGCTCTACCAGAACGATGGTT |
| | | | GCCTGTGGTGGAGTCCCTGTACGCCTTTCACCGAGGAC |
| | | | CCTCCGGCCTCCTTGGAAGTCCTGAGTGAAAGGTGTTG |
| | | | GGGCACTATGCAGGCGGTGGAGCCAGGCACCGACGAT |
| | | | GAGGGCCCCCTTCTGGAGCCCGTGGGCTCCGAGCATG |
| | | | CTCAGGACACCTACCTCGTCCTGGATAAGTGGCTCCTG |
| | | | CCCAGGAACCCTCCCTCCGAGGACCTGCCTGGCCCCG |
| | | | GAGGCAGCGTGGACATCGTTGCAATGGACGAGGGCAG |
| | | | TGAGGCTTCCTCTTGTTCAAGCGCCCTGGCCTCAAAAC |
| | | | CTAGCCCGGAAGGTGCCAGCGCCGCTAGCTTCGAGTA |
| | | | TACAATCCTCGACCCTTCCTCACAGCTCCTGAGACCAT |
| | | | GGACCCTCTGTCCGGAGTTGCCACCGACACCACCCCAT |
| | | | TTGAAGTACCTGTACCTGGTTGTGTCTGACAGCGGCAT |
| | | | CTCCACTGACTACTCTTCCGGAGATTCCCAGGGCGCCC |
| | | | AGGGCGGACTTTCCGATGGGCCTTATTCCAACCCTTAC |
| | | | GAAAACTCTCTTATCCCAGCTGCGGAGCCTCTGCCCCC |
| | | | ATCTTATGTTGCCTGCTCC (SEQ ID NO: 95) |
| GH | GHR | 2690 | ATGGACCTGTGGCAACTCCTGTTGACGCTGGCTCTGGC |
| | | | TGGTTCCTCTGACGCGTTTTCCGGAAGCGAAGCTACAG |
| | | | CAGCCATCCTGAGTAGAGCCCCCTGGAGCCTGCAGTCT |
| | | | GTCAACCCAGGACTGAAGACTAATTCTAGCAAGGAGC |
| | | | CTAAGTTCACTAAGTGCCGTAGCCCGGAACGTGAGAC |
| | | | GTTCTCCTGTCACTGGACAGATGAGGTTCATCACGGCA |
| | | | CTAAGAACTTGGGTCCCATCCAACTGTTTTATACGCGT |
| | | | AGAAACACACAAGAGTGGACGCAGGAATGGAAGGAA |
| | | | TGTCCTGACTACGTGAGCGCCGGTGAAAACAGCTGTT |
| | | | ATTTCAACTCCAGCTTCACGTCTATTTGGATTCCATACT |
| | | | GCATCAAACTGACCAGCAACGGTGGCACCGTGGATGA |
| | | | GAAGTGCTTCTCAGTCGATGAAATCGTGCAGCCAGAC |
| | | | CCTCCAATCGCCCTGAACTGGACCCTGCTCAACGTCAG |
| | | | TCTCACCGGCATCCACGCTGATATCCAGGTGAGGTGG |
| | | | GAGGCCCCTAGAAATGCGGATATCCAAAAGGGGTGGA |
| | | | TGGTGCTGGAGTATGAGCTGCAGTATAAAGAAGTGAA |
| | | | TGAGACCAAATGGAAGATGATGGACCCGATCCTTACC |
| | | | ACTTCCGTGCCCGTGTACAGCCTGAAGGTTGACAAGG |
| | | | AATACGAAGTCAGAGTGCGTAGCAAACAGAGGAACTC |
| | | | CGGTAACTATGGTGAGTTCTCTGAAGTGTTGTATGTGA |
| | | | CCCTCCCCCAGATGAGTCAGTTCACCTGCGAAGAGGA |
| | | | TTTCTACTTCCCGTGGCTCCTGATCATTATCTTTGGCAT |
| | | | CTTTGGTTTGACCGTGATGCTGTTTGTCTTTCTCTTCAG |
| | | | CAAGCAACAGCGTATCAAAATGCTGATCCTGCCGCCT |
| | | | GTGCCAGTGCCAAAGATCAAAGGCATCGACCCAGACC |
| | | | TGCTTAAGGAGGGCAAGCTGGAGGAAGTGAACACTAT |
| | | | CCTGGCCATCCACGATTCCTACAAGCCGGAGTTCCATA |
| | | | GCGACGATTCTTGGGTGGAGTTCATCGAGTTGGACATT |
| | | | GACGAGCCTGACGAGAAAACTGAGGAATCCGACACCG |
| | | | ACAGACTGCTCAGTTCCGATCATGAGAAGTCCCATAG |
| | | | CAACCTCGGTGTAAAAGATGGCGATAGTGGGCGCACC |
| | | | AGCTGTTGCGAGCCCGACATCCTGGAAACTGACTTTAA |
| | | | TGCCAACGATATTCATGAAGGCACTTCTGAGGTTGCTC |
| | | | AGCCACAGCGCCTGAAGGGCGAGGCTGATCTGTTGTG |
| | | | CTTGGACCAGAAGAATCAAAATAACAGCCCTTACCAT |
| | | | GATGCCTGTCCCGCCACTCAGCAACCTTCCGTGATCCA |
| | | | GGCAGAGAAGAACAAGCCTCAGCCACTGCCCACAGAG |
| | | | GGCGCCGAGAGCACACACCAGGCAGCCCACATCCAGC |
| | | | TGTCCAACCCATCATCCCTTTCTAACATCGACTTCTAT |
| | | | GCCCAGGTGTCTGACATTACGCCGGCCGGCTCTGTGGT |
| | | | CCTGTCTCCCGGCCAGAAAAACAAGGCCGGCATGTCT |
| | | | CAGTGTGACATGCATCCTGAGATGGTGAGTCTTTGCCA |
| | | | GGAGAACTTTCTGATGGATAACGCATATTTTTGCGAGG |
| | | | CAGACGCTAAAAAGTGTATCCCCGTGGCTCCGCACAT |
| | | | CAAAGTTGAGAGCCATATCCAGCCTTCTCTGAACCAG |
| | | | GAGGACATCTACATCACCACTGAGTCCCTGACCACTGC |
| | | | CGCAGGCCGGCCAGGCACAGGCGAGCACGTTCCCGGT |
| | | | AGCGAGATGCCTGTGCCCGACTATACTTCTATTCATAT |
| | | | CGTTCAGAGCCCACAGGGACTGATTCTGAACGCGACA |
| | | | GCTCTGCCCCTCCCCGACAAAGAATTCCTGTCCAGCTG |
| | | | TGGGTATGTGAGCACCGACCAGCTGAACAAGATCATG |
| | | | CCC (SEQ ID NO: 96) |
| PRL | PRLR | 5618 | ATGAAAGAAAATGTGGCAAGCGCTACAGTGTTCACGC |
| | | | TTCTGCTTTTTCTTAACACCTGTTTGCTGAACGGACAG |
| | | | CTGCCTCCCGGGAAGCCGGAGATTTTCAAGTGTCGCTC |
| | | | CCCCAATAAGGAGACTTTCACTTGTTGGTGGCGCCCCG |
| | | | GCACTGACGGTGGCTTGCCCACCAATTACTCCCTGACT |
| | | | TATCACCGTGAGGGAGAAACCCTGATGCACGAGTGCC |
| | | | CTGATTATATTACAGGTGGCCCCAACAGCTGTCACTTC |
| | | | GGCAAGCAGTATACCAGCATGTGGCGCACATATATTA |
| | | | TGATGGTTAATGCAACAAACCAGATGGGCAGCTCTTTC |
| | | | TCCGATGAGCTGTACGTGGACGTTACTTACATCGTGCA |
| | | | GCCCGATCCACCCCTCGAGCTGGCTGTGGAGGTGAAG |
| | | | CAGCCGGAAGATCGCAAGCCTTACCTGTGGATCAAGT |
| | | | GGAGCCCTCCCACTCTGATCGACTTGAAGACTGGTTGG |
| | | | TTTACATTGCTGTACGAAATCCGCCTGAAACCGGAAA |
| | | | AGGCAGCTGAGTGGGAAATCCACTTTGCAGGGCAGCA |
| | | | AACCGAGTTTAAGATTCTGTCCCTGCACCCGGGGCAG |
| | | | AAGTACCTGGTTCAGGTGCGTTGTAAACCTGACCATGG |
| | | | GTATTGGTCAGCTTGGAGCCCTGCTACCTTCATCCAGA |
| | | | TCCCCTCTGACTTCACCATGAACGATACTACCGTGTGG |
| | | | ATCAGCGTTGCTGTGCTCAGTGCCGTGATCTGCCTGAT |
| | | | TATCGTCTGGGCCGTGGCCCTCAAGGGCTATAGCATGG |
| | | | TGACCTGTATCTTCCCTCCAGTGCCCGGACCGAAGATC |
| | | | AAGGGATTCGACGCACACCTTCTGGAGAAGGGAAAAT |
| | | | CTGAAGAGCTTCTCTCTGCCCTGGGCTGCCAGGACTTC |
| | | | CCGCCCACCAGTGACTACGAAGATCTTCTGGTAGAAT |
| | | | ATCTGGAAGTGGATGACTCCGAGGATCAGCACCTGAT |
| | | | GAGTGTGCATTCCAAGGAACACCCCTCCCAGGGCATG |
| | | | AAACCTACGTACCTGGACCCAGATACTGATTCTGGAC |
| | | | GGGGATCCTGCGATTCACCCTCCCTGCTCAGCGAGAA |
| | | | GTGTGAAGAGCCTCAGGCCAACCCCTCCACCTTTTATG |
| | | | ACCCCGAGGTGATCGAGAAACCCGAGAATCCTGAGAC |
| | | | TACCCACACATGGGACCCGCAGTGCATCAGCATGGAG |
| | | | GGAAAGATCCCCTACTTCCATGCAGGGGGCTCCAAAT |
| | | | GCTCCACCTGGCCGCTTCCTCAGCCTTCCCAACACAAC |
| | | | CCTAGGTCTTCCTACCACAACATCACCGACGTGTGCGA |
| | | | ACTGGCTGTGGGCCCTGCCGGGGCCCCGGCAACACTG |
| | | | CTCAACGAAGCCGGTAAGGACGCCTTGAAGTCCAGTC |
| | | | AGACCATCAAGTCTCGTGAAGAGGGCAAGGCTACCCA |
| | | | ACAGAGAGAAGTTGAAAGCTTCCATTCCGAAACTGAC |
| | | | CAGGATACACCGTGGCTGCTCCCGCAGGAGAAGACCC |
| | | | CCTTCGGATCTGCCAAGCCTCTGGACTATGTCGAAATC |
| | | | CACAAGGTGAACAAAGATGGTGCACTGTCCCTCTTGC |
| | | | CCAAGCAAAGAGAAAATTCCGGCAAACCCAAAAAGCC |
| | | | AGGGACACCCGAGAATAACAAGGAGTACGCAAAAGT |
| | | | GAGCGGTGTGATGGACAATAACATCCTGGTCCTCGTTC |
| | | | CGGACCCGCACGCGAAAAACGTGGCTTGTTTCGAGGA |
| | | | ATCTGCCAAGGAAGCTCCCCCGTCCCTGGAGCAGAAC |
| | | | CAGGCTGAGAAGGCCCTGGCGAACTTCACAGCCACCT |
| | | | CTAGCAAGTGTAGGCTGCAACTGGGCGGACTCGACTA |
| | | | CCTTGATCCAGCCTGTTTTACCCACAGCTTCCAC (SEQ |
| | | | ID NO: 97) |
| IFNα/β/ω/ε/κ | IFNAR2 | 3455 | ATGCTTCTGAGCCAGAACGCATTCATCTTCCGCTCCCT |
| | | | TAACCTGGTTCTGATGGTGTATATCAGTCTGGTGTTCG |
| | | | GCATTTCTTATGACAGCCCCGACTACACCGACGAGAGT |
| | | | TGTACGTTCAAGATCAGCCTGCGGAACTTCCGCAGCAT |
| | | | TCTGAGCTGGGAACTGAAGAATCATAGCATCGTGCCC |
| | | | ACTCATTACACTTTGCTGTATACCATCATGAGCAAACC |
| | | | CGAAGACCTGAAGGTGGTAAAGAACTGTGCCAACACA |
| | | | ACGCGTTCCTTTTGCGATTTGACCGACGAGTGGCGCAG |
| | | | CACACATGAAGCCTACGTGACCGTTCTGGAGGGTTTCA |
| | | | GCGGCAACACGACACTGTTTAGCTGTAGCCACAACTTC |
| | | | TGGCTGGCTATTGACATGAGCTTCGAACCACCTGAGTT |
| | | | CGAAATCGTCGGCTTCACTAACCACATCAACGTGATG |
| | | | GTCAAGTTTCCGTCCATCGTAGAAGAGGAACTGCAGTT |
| | | | TGACCTGTCCTTGGTGATCGAGGAACAGAGTGAGGGC |
| | | | ATCGTTAAAAAGCACAAGCCCGAGATTAAGGGGAACA |
| | | | TGAGTGGGAACTTTACGTATATTATCGACAAATTGATT |
| | | | CCTAATACTAACTACTGTGTGAGCGTCTATCTCGAGCA |
| | | | CAGCGACGAACAGGCAGTGATCAAGTCCCCTCTGAAA |
| | | | TGCACCCTCCTGCCCCCTGGCCAAGAGTCCGAGTCTGC |
| | | | CGAATCCGCGAAGATCGGGGGTATTATCACCGTGTTTC |
| | | | TGATCGCTCTCGTTCTTACATCTACAATTGTGACTCTTA |
| | | | AGTGGATCGGCTACATCTGTCTGCGCAACAGTCTGCCT |
| | | | AAGGTCCTGAACTTCCATAACTTCTTGGCCTGGCCCTT |
| | | | CCCCAACTTGCCTCCCTTGGAAGCTATGGACATGGTAG |
| | | | AAGTGATCTACATCAACAGGAAAAAGAAAGTGTGGGA |
| | | | CTACAACTACGACGATGAGTCTGACAGCGACACCGAG |
| | | | GCCGCTCCCCGCACCTCTGGTGGCGGGTATACTATGCA |
| | | | CGGTCTTACTGTTCGCCCACTTGGCCAGGCCAGCGCTA |
| | | | CTAGCACCGAATCCCAGCTGATCGATCCAGAGAGCGA |
| | | | AGAGGAACCGGACCTGCCTGAAGTGGATGTGGAACTC |
| | | | CCAACCATGCCCAAGGACAGCCCCCAACAGCTGGAGT |
| | | | TGCTGTCAGGACCCTGTGAGAGACGCAAGTCACCCCT |
| | | | GCAGGACCCATTTCCCGAGGAAGACTACTCAAGCACC |
| | | | GAGGGCAGTGGCGGTCGCATCACGTTCAACGTCGACC |
| | | | TCAATAGTGTCTTCCTTCGTGTGCTCGACGATGAGGAT |
| | | | TCCGACGATCTCGAAGCTCCTCTGATGCTCTCCTCTCA |
| | | | CCTTGAAGAGATGGTAGACCCCGAAGATCCTGACAAC |
| | | | GTGCAGTCCAACCATCTGTTGGCCAGCGGAGAAGGCA |
| | | | CCCAGCCCACTTTTCCAAGCCCCTCTAGCGAGGGTCTG |
| | | | TGGTCCGAGGATGCTCCGAGCGACCAGAGCGACACCA |
| | | | GCGAGTCTGACGTGGACCTGGGGGATGGTTATATCAT |
| | | | GAGG (SEQ ID NO: 98) |
| IFNγ | IFNGR1 | 3459 | ATGGCTCTGCTCTTCCTCCTGCCCCTCGTGATGCAGGG |
| | | | CGTTTCTAGGGCCGAGATGGGAACAGCCGATCTGGGT |
| | | | CCCAGTTCTGTCCCTACCCCGACCAATGTGACTATCGA |
| | | | GTCCTACAACATGAATCCAATCGTCTACTGGGAATATC |
| | | | AGATCATGCCACAGGTACCTGTGTTTACAGTGGAAGT |
| | | | GAAGAATTACGGCGTTAAGAACTCAGAATGGATTGAC |
| | | | GCGTGCATCAACATCAGCCACCATTACTGTAACATTAG |
| | | | CGACCACGTCGGCGATCCTAGCAACTCACTGTGGGTG |
| | | | AGGGTGAAGGCTCGTGTTGGTCAAAAGGAGTCAGCCT |
| | | | ATGCGAAGTCCGAAGAGTTCGCTGTGTGTAGAGACGG |
| | | | CAAAATCGGCCCGCCAAAGTTGGACATTCGCAAGGAA |
| | | | GAGAAACAGATCATGATTGACATCTTCCACCCTTCTGT |
| | | | GTTCGTTAATGGTGATGAACAGGAGGTTGACTACGAC |
| | | | CCAGAAACGACATGCTACATCCGCGTGTATAACGTGT |
| | | | ACGTTCGCATGAACGGCTCTGAGATCCAGTATAAGAT |
| | | | CCTGACCCAGAAGGAGGACGATTGTGACGAGATCCAG |
| | | | TGCCAACTGGCAATCCCGGTGAGCTCCCTGAACTCACA |
| | | | GTATTGTGTGTCCGCAGAGGGCGTGCTGCACGTCTGGG |
| | | | GCGTGACAACCGAAAAGTCCAAAGAGGTCTGTATCAC |
| | | | CATCTTCAACTCATCCATCAAAGGCTCCCTGTGGATCC |
| | | | CTGTTGTGGCAGCTTTGCTCCTGTTCCTTGTGTTGAGCC |
| | | | TGGTCTTCATCTGCTTCTATATTAAAAAGATCAACCCT |
| | | | CTGAAGGAGAAAAGCATTATCCTGCCTAAGTCTCTGAT |
| | | | TTCTGTGGTACGCAGCGCAACTCTGGAAACAAAACCC |
| | | | GAATCCAAGTATGTGAGCCTGATTACTAGCTATCAACC |
| | | | GTTCTCTCTGGAAAAAGAAGTGGTATGCGAGGAACCT |
| | | | CTGTCTCCTGCAACCGTGCCCGGTATGCACACAGAGG |
| | | | ATAACCCTGGTAAGGTGGAACACACCGAAGAGCTGTC |
| | | | TTCCATCACCGAGGTGGTCACCACAGAAGAGAACATC |
| | | | CCCGACGTAGTGCCGGGCAGCCACTTGACCCCTATTGA |
| | | | GCGCGAGTCCTCAAGCCCCCTGTCCTCTAACCAGAGCG |
| | | | AGCCCGGGAGCATCGCCCTGAACTCCTACCATAGCCG |
| | | | GAACTGCTCCGAGTCCGATCACTCTCGCAACGGCTTTG |
| | | | ATACCGATTCATCCTGCCTGGAGAGCCATAGCTCCCTG |
| | | | TCTGATTCAGAGTTTCCCCCGAATAACAAAGGTGAGAT |
| | | | TAAGACTGAGGGCCAGGAGCTGATCACTGTGATCAAA |
| | | | GCGCCTACGAGTTTCGGCTACGACAAGCCACACGTTTT |
| | | | GGTGGATTTGCTGGTGGATGACTCAGGCAAAGAGAGC |
| | | | CTGATTGGCTACAGGCCTACTGAAGACTCTAAGGAGTT |
| | | | CAGC (SEQ ID NO: 99) |
| IFNλ1/λ2/λ3 | IFNLR1 | 163702 | ATGGCTGGTCCTGAACGCTGGGGCCCCCTGCTCCTGTG |
| | | | CTTGCTGCAGGCAGCGCCTGGACGCCCCCGTCTGGCTC |
| | | | CGCCCCAGAATGTCACACTCCTGAGCCAGAACTTCTCC |
| | | | GTGTACCTGACTTGGTTGCCCGGGCTCGGTAACCCACA |
| | | | GGACGTGACATACTTTGTTGCCTACCAATCTTCCCCAA |
| | | | CCCGGAGGCGGTGGCGTGAGGTTGAAGAGTGTGCTGG |
| | | | CACAAAAGAGCTGCTCTGCTCTATGATGTGCCTGAAA |
| | | | AAGCAGGACCTGTATAACAAGTTCAAAGGCCGCGTGA |
| | | | GGACGGTGAGTCCGAGCTCCAAGTCTCCATGGGTCGA |
| | | | ATCAGAGTACCTGGACTATCTGTTCGAAGTCGAGCCCG |
| | | | CACCGCCCGTGCTGGTTCTGACCCAGACCGAAGAGAT |
| | | | CCTGTCCGCTAACGCTACTTACCAGCTGCCTCCCTGTA |
| | | | TGCCACCTCTCGATCTGAAATACGAGGTCGCCTTCTGG |
| | | | AAGGAAGGCGCCGGTAATAAGACGTTGTTCCCCGTAA |
| | | | CTCCCCACGGCCAGCCCGTTCAGATCACCCTGCAACCT |
| | | | GCGGCCAGCGAGCATCACTGCCTCAGCGCCCGCACTA |
| | | | TCTACACCTTTAGCGTCCCCAAGTATTCCAAGTTTTCC |
| | | | AAGCCAACTTGCTTTCTTCTGGAGGTTCCCGAGGCAAA |
| | | | TTGGGCATTCCTGGTGCTGCCTTCCCTGCTCATCCTGCT |
| | | | CCTGGTGATCGCTGCGGGCGGAGTGATCTGGAAGACA |
| | | | CTGATGGGCAATCCCTGGTTCCAACGCGCAAAAATGC |
| | | | CGCGCGCCCTGGACTTTTCAGGCCATACGCATCCTGTC |
| | | | GCCACGTTCCAGCCTAGCCGCCCCGAGAGTGTGAACG |
| | | | ATCTTTTTCTGTGCCCCCAAAAAGAGTTGACCCGCGGT |
| | | | GTGCGTCCCACACCTCGGGTTAGAGCCCCAGCAACTC |
| | | | AACAGACACGCTGGAAAAAGGATCTGGCGGAGGACG |
| | | | AAGAGGAAGAGGATGAAGAGGACACCGAAGACGGCG |
| | | | TTAGTTTCCAGCCCTATATCGAACCCCCTAGCTTCCTC |
| | | | GGCCAAGAGCACCAGGCCCCCGGACACAGTGAAGCCG |
| | | | GCGGAGTGGACTCCGGACGTCCCAGAGCACCACTGGT |
| | | | GCCCTCTGAAGGCTCCAGCGCTTGGGATTCCAGCGATC |
| | | | GTTCCTGGGCCAGTACCGTGGACTCTTCCTGGGATCGC |
| | | | GCCGGATCCTCAGGTTACTTGGCCGAAAAGGGCCCTG |
| | | | GCCAGGGCCCCGGCGGAGACGGCCACCAAGAGTCCCT |
| | | | GCCCCCACCTGAGTTCAGTAAGGACTCTGGTTTTTTGG |
| | | | AAGAGCTGCCCGAAGACAATCTCTCCTCTTGGGCCACC |
| | | | TGGGGCACCCTGCCTCCGGAGCCAAACCTCGTGCCGG |
| | | | GTGGACCCCCTGTGAGCCTGCAGACCCTTACTTTTTGC |
| | | | TGGGAGAGCTCCCCAGAGGAAGAGGAAGAGGCACGC |
| | | | GAGTCCGAGATTGAAGATAGCGACGCGGGCAGCTGGG |
| | | | GTGCTGAATCCACTCAGCGTACAGAGGACAGGGGTCG |
| | | | CACCCTGGGCCACTACATGGCTCGC (SEQ ID NO: 100) |
| IL26/19/20/24 | IL20Ra | 53832 | ATGCGCGCTCCCGGCCGTCCTGCCCTGCGCCCGCTGCC |
| | | | CCTGCCCCCACTGCTTCTGCTTCTCCTGGCTGCCCCTTG |
| | | | GGGCCGTGCGGTTCCTTGCGTGAGCGGGGGACTGCCT |
| | | | AAGCCCGCTAACATCACATTCCTCTCCATCAATATGAA |
| | | | GAACGTCCTGCAGTGGACACCCCCGGAGGGACTGCAG |
| | | | GGCGTCAAAGTGACATACACCGTCCAGTACTTCATCTA |
| | | | CGGGCAAAAAAAGTGGCTGAACAAATCCGAATGTCGG |
| | | | AACATTAACCGCACCTACTGTGATCTGTCAGCGGAGA |
| | | | CCTCAGACTACGAGCACCAATATTACGCCAAGGTGAA |
| | | | GGCCATCTGGGGTACTAAATGCTCAAAGTGGGCCGAG |
| | | | TCTGGTCGTTTCTATCCTTTTCTTGAGACCCAGATCGGC |
| | | | CCTCCAGAGGTGGCTCTGACCACGGACGAAAAAAGTA |
| | | | TTAGTGTGGTCCTGACCGCCCCTGAAAAATGGAAGCG |
| | | | CAACCCTGAGGATCTGCCGGTGAGCATGCAACAGATT |
| | | | TACTCCAATCTCAAGTATAACGTGTCAGTGCTGAACAC |
| | | | GAAATCCAACCGCACTTGGTCACAGTGTGTGACCAAT |
| | | | CACACTCTGGTGCTGACCTGGTTGGAGCCCAACACCCT |
| | | | CTACTGCGTACACGTAGAGTCCTTCGTGCCCGGACCTC |
| | | | CCCGGCGTGCTCAACCCTCCGAAAAGCAGTGCGCCAG |
| | | | AACACTGAAAGACCAGAGCTCCGAATTCAAGGCTAAG |
| | | | ATTATCTTCTGGTACGTGCTCCCTGTGTCTATCACAGT |
| | | | GTTCCTGTTCTCTGTGATGGGCTATTCTATCTATCGCTA |
| | | | CATCCATGTCGGTAAGGAGAAGCACCCTGCAAACCTT |
| | | | ATCTTGATCTACGGTAACGAATTCGACAAGCGCTTTTT |
| | | | CGTCCCTGCAGAGAAGATCGTGATTAACTTCATCACTC |
| | | | TGAACATCTCAGATGACAGCAAGATTTCTCACCAGGA |
| | | | CATGAGCCTTCTGGGCAAGTCCAGTGATGTGAGTTCCT |
| | | | TGAACGACCCCCAGCCTTCCGGCAACCTGAGGCCCCCT |
| | | | CAGGAGGAAGAGGAAGTCAAGCACTTGGGCTACGCAT |
| | | | CCCACCTCATGGAGATCTTTTGTGACTCCGAAGAGAAC |
| | | | ACTGAGGGTACATCCCTGACCCAACAGGAATCCCTGT |
| | | | CACGCACCATTCCTCCCGACAAAACAGTGATCGAGTA |
| | | | CGAGTATGACGTTAGAACTACCGATATCTGTGCCGGTC |
| | | | CCGAGGAACAGGAGCTCAGCCTGCAAGAGGAAGTTAG |
| | | | CACCCAGGGAACACTGCTCGAGTCCCAGGCTGCCCTC |
| | | | GCTGTGCTGGGGCCCCAGACCCTTCAGTACAGCTATAC |
| | | | CCCTCAGCTGCAAGACTTGGATCCGCTGGCCCAGGAG |
| | | | CATACTGACTCCGAGGAAGGACCCGAGGAAGAGCCCA |
| | | | GTACAACCCTGGTGGATTGGGACCCGCAGACCGGTCG |
| | | | TCTGTGCATCCCCTCCCTGAGCTCATTCGACCAGGACT |
| | | | CTGAAGGCTGCGAACCCTCCGAGGGGGACGGTCTGGG |
| | | | CGAGGAAGGTCTGTTGAGCCGCCTGTATGAAGAGCCT |
| | | | GCCCCTGATCGGCCACCGGGCGAGAATGAAACCTACC |
| | | | TGATGCAGTTTATGGAAGAGTGGGGCCTGTACGTTCA |
| | | | GATGGAGAAC (SEQ ID NO: 101) |
| IL22/20/24 | IL22R | 58985 | ATGCGCACCTTGCTGACTATCCTGACCGTGGGCTCCCT |
| | | | GGCCGCTCACGCCCCTGAGGATCCCTCCGATTTGCTGC |
| | | | AGCATGTCAAGTTCCAGAGCTCCAACTTTGAGAACATC |
| | | | CTGACCTGGGACTCAGGTCCCGAGGGTACCCCCGACA |
| | | | CTGTGTACTCCATCGAGTATAAGACCTATGGCGAGCGC |
| | | | GATTGGGTGGCTAAGAAAGGCTGTCAGCGTATTACCC |
| | | | GCAAGAGTTGTAACCTGACCGTGGAGACGGGCAACCT |
| | | | GACCGAACTGTACTATGCCAGGGTGACGGCTGTAAGC |
| | | | GCTGGGGGCCGGTCCGCTACCAAGATGACCGACCGGT |
| | | | TCTCCTCTCTCCAGCACACTACCCTCAAGCCGCCCGAC |
| | | | GTGACCTGTATCTCCAAGGTGCGTAGCATCCAGATGAT |
| | | | TGTGCATCCAACCCCCACCCCCATTCGGGCTGGAGACG |
| | | | GCCATCGCCTGACCCTGGAGGACATTTTCCATGACCTG |
| | | | TTCTACCACCTGGAGCTGCAGGTTAATCGCACCTATCA |
| | | | GATGCACCTGGGTGGCAAGCAGAGGGAGTACGAATTC |
| | | | TTTGGACTGACCCCCGATACTGAGTTTCTGGGTACCAT |
| | | | CATGATCTGCGTACCAACGTGGGCAAAGGAGTCCGCT |
| | | | CCCTACATGTGCCGCGTCAAGACACTCCCTGATAGAAC |
| | | | CTGGACATACTCTTTCAGCGGGGCTTTCCTCTTCAGCA |
| | | | TGGGTTTCCTGGTTGCCGTCCTGTGCTACCTGTCTTATC |
| | | | GCTACGTGACCAAGCCGCCAGCGCCGCCAAACTCACT |
| | | | GAACGTGCAGAGGGTGCTCACCTTTCAGCCTCTCAGGT |
| | | | TCATCCAGGAGCATGTGCTTATCCCCGTGTTCGACCTC |
| | | | TCAGGTCCCTCCTCTCTGGCCCAGCCGGTGCAGTATTC |
| | | | CCAGATCAGAGTGTCCGGTCCTCGGGAGCCTGCTGGC |
| | | | GCACCCCAGCGTCATTCCCTGAGCGAAATCACCTACCT |
| | | | GGGCCAGCCTGACATCAGCATCCTCCAGCCGTCCAAT |
| | | | GTACCTCCCCCACAGATCCTTAGCCCTCTGTCCTACGC |
| | | | GCCAAACGCAGCCCCTGAAGTTGGCCCACCTAGCTAC |
| | | | GCTCCCCAGGTAACGCCTGAGGCGCAGTTCCCCTTCTA |
| | | | TGCGCCCCAGGCCATCAGTAAGGTTCAGCCCTCCAGCT |
| | | | ACGCACCTCAGGCTACACCTGATTCCTGGCCTCCATCC |
| | | | TACGGCGTGTGCATGGAGGGCAGCGGAAAGGACAGCC |
| | | | CTACTGGAACTCTGTCTTCCCCTAAGCACCTCCGTCCA |
| | | | AAAGGCCAGTTGCAGAAGGAGCCTCCCGCTGGTTCTT |
| | | | GCATGCTCGGGGGCCTCTCTCTGCAGGAGGTGACGTCC |
| | | | CTGGCCATGGAAGAGTCCCAAGAGGCAAAAAGCCTGC |
| | | | ACCAGCCTCTGGGCATCTGCACCGATCGCACCAGCGA |
| | | | TCCCAATGTCTTGCACAGCGGCGAAGAGGGGACCCCG |
| | | | CAGTACCTGAAAGGGCAACTGCCCCTGCTCTCCAGCGT |
| | | | GCAGATCGAAGGTCACCCGATGAGCCTGCCCCTTCAG |
| | | | CCCCCTAGCCGCCCTTGTAGCCCTAGTGACCAGGGACC |
| | | | TTCCCCCTGGGGCCTCTTGGAGTCTCTGGTATGTCCAA |
| | | | AGGATGAAGCGAAGTCCCCTGCCCCAGAGACGTCTGA |
| | | | TCTGGAACAGCCTACGGAACTCGACAGCCTGTTTCGCG |
| | | | GTCTGGCGTTGACTGTTCAGTGGGAGAGT (SEQ ID NO: |
| | | | 102) |
| STAT1 | minimal STAT1 | N/A | TACxxxxxxCCC (SEQ ID NO: 103) |
| | binding motif | | |
| STAT3 | minimal STAT3 | N/A | TACxxxxxxCAG (SEQ ID NO: 104) |
| | binding motif | | |
| STAT5 | minimal | N/A | TACxxxxxxCTC (SEQ ID NO: 105) |
| | STAT5a | | |
| | binding motif | | |
| STAT6 | minimal STAT6 | N/A | TACxxxxxxTTC (SEQ ID NO: 106) |
| | binding motif | | |
| CD40 | CD40 | 958 | ATGGTCCGCCTGCCACTGCAGTGCGTGCTGTGGGGCTG |
| | | | CTTGCTGACCGCGGTACACCCGGAGCCTCCCACTGCCT |
| | | | GCCGCGAGAAGCAGTACCTTATCAATTCTCAATGTTGC |
| | | | TCTCTGTGCCAGCCAGGCCAAAAACTGGTGAGCGACT |
| | | | GCACAGAGTTTACCGAGACCGAGTGTCTTCCTTGTGGA |
| | | | GAGAGCGAGTTCCTGGATACATGGAACCGCGAGACAC |
| | | | ACTGCCATCAGCACAAGTACTGTGACCCTAACCTGGG |
| | | | ACTGCGGGTCCAACAGAAGGGCACTTCAGAAACAGAT |
| | | | ACCATCTGTACGTGTGAAGAGGGGTGGCATTGCACCA |
| | | | GCGAGGCCTGCGAGTCTTGTGTCCTGCACAGAAGCTG |
| | | | CTCTCCTGGTTTCGGGGTCAAACAGATCGCTACCGGCG |
| | | | TCAGCGACACAATCTGCGAGCCCTGTCCCGTGGGGTTT |
| | | | TTCTCCAACGTGTCTTCCGCCTTCGAGAAGTGTCACCC |
| | | | CTGGACCAGCTGCGAGACAAAAGACCTGGTTGTGCAA |
| | | | CAGGCGGGCACCAACAAGACCGATGTGGTCTGCGGTC |
| | | | CTCAGGATCGTCTTAGAGCTCTGGTAGTGATTCCTATT |
| | | | ATCTTCGGCATCCTGTTTGCCATCTTGCTGGTACTGGT |
| | | | GTTTATTAAAAAGGTGGCGAAGAAACCCACCAACAAG |
| | | | GCCCCGCATCCTAAGCAGGAACCCCAGGAAATTAACT |
| | | | TTCCCGATGACCTCCCAGGGTCTAATACCGCGGCACCC |
| | | | GTCCAGGAGACGCTCCACGGGTGTCAGCCTGTGACCC |
| | | | AGGAAGACGGCAAGGAAAGCCGCATTTCCGTGCAGGA |
| | | | GCGGCAG (SEQ ID NO: 107) |
| 4-1BB | 4-1BB | 3604 | ATGGGCAACTCTTGTTACAACATCGTCGCAACACTCCT |
| | | | GTTGGTGCTTAATTTCGAACGCACCCGCAGCCTCCAGG |
| | | | ATCCATGTTCAAACTGCCCTGCCGGTACTTTCTGCGAT |
| | | | AATAACAGAAACCAGATCTGTAGCCCATGTCCCCCAA |
| | | | ACTCTTTCTCCAGTGCGGGAGGTCAACGCACATGTGAC |
| | | | ATCTGTAGGCAGTGCAAAGGAGTGTTCAGGACTAGAA |
| | | | AGGAGTGCTCATCCACAAGCAACGCTGAATGCGACTG |
| | | | CACGCCAGGGTTTCACTGTCTGGGTGCCGGCTGTTCTA |
| | | | TGTGTGAACAGGATTGTAAGCAGGGACAGGAGCTGAC |
| | | | TAAGAAAGGTTGTAAGGACTGTTGCTTCGGCACCTTCA |
| | | | ACGATCAGAAGAGGGGTATCTGTCGCCCCTGGACCAA |
| | | | CTGCAGCCTTGACGGCAAATCTGTGCTGGTCAATGGCA |
| | | | CCAAGGAACGTGACGTGGTCTGCGGTCCCTCTCCCGCC |
| | | | GACCTGTCCCCAGGGGCCAGCTCTGTGACCCCTCCGGC |
| | | | GCCCGCCCGCGAACCTGGTCATTCTCCCCAAATTATCA |
| | | | GTTTTTTCCTGGCGCTGACGAGCACTGCCTTGCTGTTC |
| | | | CTGTTGTTTTTCCTGACCCTGAGATTCTCAGTTGTGAA |
| | | | GCGTGGCAGGAAAAAGTTGCTGTATATTTTCAAACAG |
| | | | CCTTTCATGAGACCAGTGCAAACCACGCAGGAAGAGG |
| | | | ATGGCTGTTCCTGCCGCTTCCCTGAAGAGGAAGAGGG |
| | | | CGGGTGCGAGTTG (SEQ ID NO: 108) |
| TLR4 | MyD88 | 4615 | ATGGCGGCTGGGGGCCCTGGAGCCGGTTCTGCAGCCC |
| | | | CCGTGTCATCCACTAGTAGCCTGCCCCTGGCGGCTCTG |
| | | | AATATGCGCGTGCGGCGTCGCCTCAGTTTGTTCCTCAA |
| | | | TGTGAGGACTCAGGTGGCGGCAGATTGGACAGCTCTT |
| | | | GCCGAAGAGATGGATTTTGAGTACCTCGAGATCCGCC |
| | | | AGCTGGAAACTCAGGCCGACCCTACCGGACGCCTGCT |
| | | | CGACGCTTGGCAGGGTCGCCCTGGTGCCTCTGTGGGCC |
| | | | GTCTTCTGGAATTGCTCACCAAGCTTGGTCGCGACGAT |
| | | | GTACTCCTGGAGCTGGGACCTTCAATTGAAGAGGATT |
| | | | GTCAGAAGTACATCCTGAAACAGCAACAGGAAGAGGC |
| | | | CGAGAAGCCGCTGCAGGTCGCCGCAGTTGACTCTTCA |
| | | | GTGCCAAGGACTGCCGAGCTGGCAGGAATCACGACCC |
| | | | TGGATGACCCCCTCGGCCACATGCCTGAGCGGTTCGAT |
| | | | GCCTTCATTTGTTACTGCCCTTCAGATATCCAGTTCGTG |
| | | | CAAGAGATGATCAGACAGCTGGAGCAGACCAACTACC |
| | | | GGCTGAAGTTGTGCGTGTCCGATCGCGACGTGTTGCCG |
| | | | GGCACCTGCGTGTGGTCAATCGCCAGCGAGCTGATTG |
| | | | AGAAGCGCTGTCGGCGTATGGTCGTGGTAGTGTCCGA |
| | | | TGACTATTTGCAGAGTAAGGAGTGCGACTTCCAGACC |
| | | | AAATTCGCCTTGAGCCTTTCCCCCGGTGCTCATCAGAA |
| | | | ACGTCTGATTCCAATCAAGTACAAGGCCATGAAAAAG |
| | | | GAATTCCCTAGTATTCTTCGCTTTATTACTGTTTGTGAC |
| | | | TACACCAATCCATGCACCAAGTCCTGGTTCTGGACGCG |
| | | | CCTGGCCAAAGCGCTGTCACTGCCA (SEQ ID NO: 109) |
| OX40 | OX40 | 7293 | ATGTGTGTGGGCGCACGCAGGCTGGGCAGGGGCCCCT |
| | | | GCGCTGCACTTCTGCTCCTGGGGCTGGGTTTGTCCACC |
| | | | GTGACCGGACTGCACTGCGTCGGCGACACCTATCCAA |
| | | | GCAACGACAGGTGTTGCCATGAGTGTAGACCAGGCAA |
| | | | TGGTATGGTGAGCCGCTGCAGTCGCAGCCAGAATACC |
| | | | GTGTGTAGACCTTGTGGTCCTGGCTTTTATAACGACGT |
| | | | GGTCAGTTCCAAACCGTGTAAACCGTGTACGTGGTGC |
| | | | AATCTGCGTAGCGGATCTGAACGCAAGCAGCTCTGCA |
| | | | CCGCCACGCAGGACACGGTCTGTCGCTGCCGCGCTGG |
| | | | CACCCAGCCGTTGGACAGTTACAAACCAGGTGTTGAC |
| | | | TGTGCACCTTGTCCCCCAGGCCACTTCAGCCCGGGAGA |
| | | | CAATCAGGCCTGCAAACCCTGGACCAACTGCACCCTG |
| | | | GCTGGTAAACATACTCTCCAGCCTGCATCCAACAGCTC |
| | | | TGATGCTATCTGCGAGGACCGCGATCCCCCTGCCACCC |
| | | | AGCCCCAGGAGACCCAAGGTCCACCTGCCCGCCCTAT |
| | | | CACCGTGCAGCCCACTGAAGCCTGGCCAAGGACCTCA |
| | | | CAGGGTCCTTCCACACGGCCGGTGGAGGTGCCTGGAG |
| | | | GCAGAGCCGTCGCTGCGATCCTCGGTCTTGGCCTCGTG |
| | | | CTGGGTCTCCTGGGACCTCTCGCTATCCTCCTGGCCCT |
| | | | GTATCTGCTTAGAAGGGATCAGCGCCTGCCCCCTGATG |
| | | | CCCATAAGCCGCCAGGGGGTGGCTCTTTTCGCACCCCA |
| | | | ATTCAGGAGGAACAGGCCGATGCCCACTCTACTCTGG |
| | | | CTAAGATC (SEQ ID NO: 110) |
| CD30 | CD30 | 943 | ATGCGCGTGCTGCTCGCCGCTCTTGGCTTGCTGTTCCT |
| | | | GGGTGCTCTGCGTGCTTTCCCCCAGGACAGACCCTTCG |
| | | | AGGACACATGCCACGGCAACCCCAGCCATTACTATGA |
| | | | CAAAGCAGTGCGTCGCTGCTGTTACCGTTGTCCGATGG |
| | | | GGCTGTTCCCTACGCAACAGTGCCCCCAGAGACCCAC |
| | | | CGATTGTCGTAAGCAGTGCGAACCTGATTATTACCTGG |
| | | | ACGAGGCCGATAGGTGCACAGCCTGCGTGACATGCTC |
| | | | CCGTGACGATCTGGTAGAAAAAACTCCCTGTGCTTGG |
| | | | AACAGCTCCAGGGTGTGCGAGTGTCGCCCTGGCATGTT |
| | | | CTGTTCTACCAGCGCAGTGAACAGTTGTGCGAGGTGTT |
| | | | TCTTTCACAGCGTCTGCCCGGCCGGCATGATCGTGAAG |
| | | | TTCCCAGGCACCGCCCAAAAGAACACCGTCTGTGAAC |
| | | | CTGCCTCTCCTGGTGTAAGCCCAGCTTGCGCCTCACCC |
| | | | GAGAACTGCAAAGAGCCCTCAAGCGGCACCATCCCTC |
| | | | AGGCCAAGCCCACACCCGTATCCCCTGCGACGTCCTCT |
| | | | GCCTCCACCATGCCTGTCAGAGGAGGCACCCGTTTGGC |
| | | | TCAGGAGGCGGCCAGCAAGCTGACCCGCGCTCCTGAC |
| | | | AGCCCCAGTAGCGTGGGACGCCCATCTAGCGATCCGG |
| | | | GCTTGTCTCCGACGCAACCTTGCCCTGAGGGCTCCGGC |
| | | | GACTGTCGTAAGCAGTGTGAACCGGATTATTACCTGG |
| | | | ACGAGGCTGGCCGCTGCACCGCATGTGTCTCCTGTAGC |
| | | | CGCGATGACCTGGTAGAGAAGACACCATGCGCCTGGA |
| | | | ACTCTTCCCGTACCTGCGAGTGCAGACCAGGAATGATT |
| | | | TGTGCCACGAGCGCAACCAATTCCTGCGCGCGCTGTGT |
| | | | GCCCTATCCCATCTGTGCGGCTGAGACCGTCACGAAGC |
| | | | CTCAGGATATGGCCGAGAAAGACACCACGTTCGAAGC |
| | | | ACCCCCTCTGGGTACGCAGCCCGACTGTAACCCTACCC |
| | | | CAGAAAATGGCGAAGCGCCGGCAAGTACCTCTCCAAC |
| | | | TCAATCCCTCCTGGTCGACAGCCAGGCCTCCAAGACCC |
| | | | TCCCTATCCCCACCTCCGCCCCCGTCGCACTGAGCTCC |
| | | | ACCGGTAAGCCCGTCCTGGATGCCGGTCCAGTTTTGTT |
| | | | CTGGGTGATCCTGGTGCTGGTCGTTGTGGTCGGATCTA |
| | | | GTGCCTTTTTGCTGTGTCATCGTCGCGCTTGTCGCAAA |
| | | | CGTATCCGTCAGAAGCTGCACCTCTGCTATCCTGTGCA |
| | | | GACTAGTCAGCCGAAGCTGGAACTGGTGGACAGCAGA |
| | | | CCCAGGCGTTCAAGCACCCAGCTGCGCTCTGGCGCATC |
| | | | CGTGACTGAACCAGTTGCAGAGGAACGTGGTCTCATG |
| | | | TCCCAGCCTCTGATGGAAACCTGCCACTCTGTGGGCGC |
| | | | CGCTTATTTGGAATCTCTCCCTCTCCAGGACGCCTCAC |
| | | | CGGCTGGCGGACCTTCTAGCCCCCGCGACCTGCCGGA |
| | | | GCCAAGGGTTAGCACGGAGCACACCAATAACAAGATC |
| | | | GAAAAGATCTACATCATGAAGGCAGACACAGTTATTG |
| | | | TGGGCACAGTTAAGGCGGAACTGCCTGAAGGCCGCGG |
| | | | GCTGGCGGGACCGGCTGAGCCCGAGTTGGAAGAGGAA |
| | | | CTGGAGGCCGATCACACACCCCACTACCCCGAGCAGG |
| | | | AAACCGAGCCTCCCCTCGGTAGCTGCAGTGACGTAAT |
| | | | GCTGTCTGTGGAAGAGGAAGGCAAGGAGGACCCTCTG |
| | | | CCTACTGCAGCCTCTGGAAAG (SEQ ID NO: 111) |
| TNFa | TNFR1 | 7132 | ATGGGTCTGTCTACAGTGCCCGACCTGCTCCTGCCCCT |
| | | | GGTGCTTCTCGAACTCCTGGTGGGAATTTATCCCAGCG |
| | | | GCGTGATCGGTCTCGTGCCCCACTTGGGCGATAGGGA |
| | | | GAAACGCGATAGTGTCTGCCCCCAGGGGAAGTACATC |
| | | | CACCCCCAAAATAACTCCATCTGCTGTACCAAGTGTCA |
| | | | TAAGGGTACCTACCTGTACAACGACTGTCCTGGTCCAG |
| | | | GACAGGACACGGATTGCCGCGAATGTGAGAGCGGTTC |
| | | | CTTCACGGCTAGCGAGAACCATCTCAGGCATTGTCTCT |
| | | | CCTGCAGCAAGTGTAGAAAGGAAATGGGTCAGGTGGA |
| | | | GATTTCTTCCTGCACTGTGGACCGTGACACGGTGTGCG |
| | | | GCTGTCGCAAAAACCAGTACAGGCATTATTGGTCAGA |
| | | | GAACCTGTTCCAGTGCTTCAACTGTAGCCTCTGTCTGA |
| | | | ACGGCACGGTGCACCTGTCATGTCAGGAGAAGCAGAA |
| | | | CACTGTGTGTACATGCCATGCCGGCTTTTTCCTGAGAG |
| | | | AGAATGAGTGTGTGAGTTGTAGCAATTGCAAAAAGTC |
| | | | CCTGGAATGCACAAAGCTGTGTTTGCCACAGATCGAG |
| | | | AATGTGAAGGGTACCGAGGATTCTGGAACCACAGTGC |
| | | | TGCTTCCACTCGTAATCTTCTTTGGCTTGTGTTTGCTGT |
| | | | CTCTGCTCTTCATCGGTCTCATGTATCGCTATCAGCGTT |
| | | | GGAAGAGCAAGCTGTATTCCATCGTGTGCGGTAAGAG |
| | | | CACTCCCGAGAAGGAAGGAGAACTGGAGGGCACAAC |
| | | | CACAAAGCCCTTGGCCCCTAACCCGAGCTTCTCTCCAA |
| | | | CTCCAGGATTCACCCCAACACTGGGCTTCTCTCCTGTG |
| | | | CCGTCCAGCACCTTCACTTCTTCCAGCACATACACCCC |
| | | | CGGAGATTGTCCAAATTTCGCCGCTCCTCGTCGCGAAG |
| | | | TGGCGCCGCCTTATCAGGGTGCCGACCCGATTCTGGCA |
| | | | ACCGCACTGGCCTCAGACCCAATCCCTAACCCTCTCCA |
| | | | GAAGTGGGAGGACTCCGCTCATAAACCTCAGAGTCTC |
| | | | GACACCGATGACCCGGCCACCCTGTACGCCGTCGTGG |
| | | | AGAACGTTCCCCCACTGAGATGGAAGGAGTTCGTGCG |
| | | | TCGCCTCGGCTTGTCTGACCACGAAATTGACAGGTTGG |
| | | | AGCTGCAAAATGGTAGGTGCCTCCGCGAGGCACAATA |
| | | | CTCCATGCTCGCGACATGGAGGCGCCGGACCCCGAGG |
| | | | CGCGAAGCCACTCTGGAGCTCCTGGGACGTGTACTGC |
| | | | GTGACATGGATCTCCTGGGTTGCCTGGAAGACATCGA |
| | | | AGAGGCCCTGTGCGGCCCTGCGGCTCTGCCTCCCGCAC |
| | | | CCTCTTTGCTGAGG (SEQ ID NO: 112) |
| TNFa | TNFR2 | 7133 | ATGGCCCCTGTAGCAGTGTGGGCGGCCCTGGCCGTGG |
| | | | GGCTGGAGCTCTGGGCCGCGGCCCATGCCCTGCCCGCT |
| | | | CAAGTGGCATTCACTCCCTACGCGCCCGAGCCCGGCTC |
| | | | CACCTGCAGGCTGCGTGAGTATTACGACCAGACCGCT |
| | | | CAGATGTGTTGCAGTAAGTGTAGCCCTGGCCAGCACG |
| | | | CCAAGGTGTTCTGTACCAAGACGTCTGATACCGTGTGC |
| | | | GACTCTTGTGAAGATTCCACCTACACCCAGCTGTGGAA |
| | | | CTGGGTGCCTGAGTGCTTGTCTTGTGGCTCTCGCTGCA |
| | | | GCTCAGACCAGGTAGAGACCCAGGCCTGTACCCGCGA |
| | | | GCAGAACCGCATCTGTACCTGCAGGCCCGGCTGGTAC |
| | | | TGCGCTCTGTCCAAGCAGGAGGGGTGCAGACTGTGCG |
| | | | CGCCCCTCCGGAAGTGCCGTCCCGGCTTTGGAGTGGCA |
| | | | CGTCCTGGTACCGAGACATCTGACGTGGTTTGCAAGCC |
| | | | ATGCGCCCCTGGGACCTTCTCTAACACCACATCCAGCA |
| | | | CGGACATTTGCAGACCTCATCAGATTTGTAACGTCGTG |
| | | | GCCATCCCTGGCAACGCCTCCATGGATGCCGTCTGCAC |
| | | | CTCCACCTCTCCGACCCGCAGTATGGCCCCCGGCGCAG |
| | | | TGCACCTGCCGCAGCCCGTATCAACCCGCTCCCAACAT |
| | | | ACTCAGCCTACACCAGAGCCTAGCACCGCCCCCTCCAC |
| | | | ATCCTTCTTGCTGCCTATGGGCCCGAGCCCTCCCGCTG |
| | | | AAGGCTCCACAGGTGACTTCGCGCTCCCGGTGGGACT |
| | | | GATCGTGGGCGTCACTGCCCTCGGCCTGCTCATCATTG |
| | | | GTGTTGTGAATTGTGTCATCATGACCCAGGTTAAGAAA |
| | | | AAGCCCCTGTGTCTGCAGCGTGAAGCTAAGGTCCCGC |
| | | | ACCTGCCGGCGGACAAGGCACGCGGCACTCAAGGGCC |
| | | | TGAGCAACAGCACCTCCTGATCACCGCCCCCAGTAGCT |
| | | | CCAGTTCTTCCCTCGAGAGCTCTGCCTCTGCCCTGGAT |
| | | | AGGCGTGCGCCAACACGCAACCAGCCGCAGGCCCCAG |
| | | | GCGTGGAGGCCTCAGGAGCAGGGGAGGCACGCGCTTC |
| | | | CACCGGCAGCTCCGACTCCTCTCCTGGCGGTCACGGAA |
| | | | CCCAGGTGAATGTCACTTGTATTGTCAACGTGTGTAGC |
| | | | TCCTCTGATCACTCTAGCCAGTGCTCTTCCCAGGCGAG |
| | | | TAGCACTATGGGGGACACTGACTCCAGCCCCTCCGAG |
| | | | AGTCCCAAGGACGAGCAGGTCCCGTTCTCCAAGGAAG |
| | | | AGTGTGCGTTTCGGTCCCAGCTCGAAACTCCAGAGAC |
| | | | GCTTCTGGGATCCACGGAAGAGAAGCCCCTGCCCCTG |
| | | | GGTGTCCCAGACGCGGGCATGAAGCCATCC (SEQ ID |
| | | | NO: 113) |
| FLT3L | FLT3 | 2322 | ATGCCTGCGCTGGCGAGGGATGGAGGCCAGTTGCCCT |
| | | | TGCTGGTGGTTTTCAGTGCAATGATCTTCGGTACCATC |
| | | | ACAAACCAAGACCTCCCCGTCATCAAATGTGTCCTCAT |
| | | | CAACCACAAAAACAATGACTCATCTGTGGGGAAGAGC |
| | | | TCTTCCTATCCAATGGTTTCTGAAAGCCCAGAGGACCT |
| | | | TGGCTGCGCGCTCCGCCCTCAGTCCAGTGGTACGGTCT |
| | | | ACGAGGCTGCCGCTGTGGAGGTTGACGTCAGTGCTTCC |
| | | | ATCACCCTTCAGGTCCTGGTTGACGCTCCTGGCAACAT |
| | | | CTCCTGTTTGTGGGTGTTCAAGCACAGCTCCCTGAACT |
| | | | GCCAGCCTCATTTTGACCTCCAAAACAGAGGCGTTGTG |
| | | | TCCATGGTCATCTTGAAGATGACCGAAACCCAGGCAG |
| | | | GGGAATACCTCCTGTTCATCCAATCCGAAGCGACCAA |
| | | | CTACACCATCCTTTTCACCGTCTCTATCAGGAACACCC |
| | | | TCCTGTATACTCTGCGTCGCCCTTACTTTCGCAAAATG |
| | | | GAGAACCAGGACGCTCTGGTGTGTATCAGCGAGTCTG |
| | | | TTCCTGAGCCTATTGTGGAGTGGGTTCTCTGTGACAGC |
| | | | CAGGGCGAGTCCTGTAAGGAGGAATCCCCCGCTGTGG |
| | | | TTAAAAAGGAAGAGAAGGTACTGCACGAGCTGTTCGG |
| | | | CACCGACATTCGTTGCTGTGCACGGAACGAGCTGGGC |
| | | | CGCGAATGCACTCGGCTTTTCACCATCGATCTCAACCA |
| | | | GACGCCTCAGACTACCCTGCCCCAACTGTTCCTCAAAG |
| | | | TGGGGGAGCCCCTGTGGATTCGTTGCAAGGCCGTCCA |
| | | | CGTCAACCATGGTTTCGGTCTGACCTGGGAATTGGAGA |
| | | | ACAAAGCTCTGGAGGAAGGTAACTACTTTGAAATGTC |
| | | | TACCTACTCTACCAACCGCACTATGATCAGGATCCTCT |
| | | | TCGCCTTCGTGAGCTCCGTGGCCCGGAATGACACTGGC |
| | | | TATTACACATGCAGCTCCTCTAAGCACCCATCCCAAAG |
| | | | CGCGCTGGTGACCATCGTAGAGAAGGGCTTTATTAAC |
| | | | GCTACCAACTCTTCCGAGGACTACGAGATCGACCAAT |
| | | | ACGAAGAGTTCTGTTTCTCCGTGCGCTTCAAAGCTTAC |
| | | | CCGCAGATTCGCTGTACATGGACCTTTTCTAGAAAGAG |
| | | | CTTCCCGTGCGAGCAGAAGGGTTTGGACAACGGCTAC |
| | | | TCAATCTCCAAATTCTGTAACCACAAGCACCAACCGG |
| | | | GCGAGTACATCTTTCATGCTGAGAATGATGACGCCCA |
| | | | GTTTACCAAAATGTTCACCCTGAACATTCGTCGCAAGC |
| | | | CTCAGGTCCTCGCAGAGGCTAGTGCCTCCCAGGCGTCC |
| | | | TGTTTCTCCGATGGTTACCCTCTCCCTAGCTGGACCTG |
| | | | GAAAAAGTGTTCTGACAAATCCCCTAACTGCACAGAG |
| | | | GAAATCACTGAGGGTGTCTGGAATCGCAAGGCTAACC |
| | | | GCAAGGTGTTTGGCCAGTGGGTCTCTTCCAGTACCCTC |
| | | | AACATGAGCGAAGCCATCAAGGGCTTTCTGGTCAAGT |
| | | | GTTGCGCGTACAATTCCCTGGGCACCAGCTGTGAGACC |
| | | | ATTCTGCTTAACAGCCCCGGACCTTTTCCTTTCATCCA |
| | | | GGATAACATTAGTTTTTACGCTACAATTGGAGTGTGTT |
| | | | TGCTGTTTATCGTGGTTCTGACTTTGCTGATCTGTCACA |
| | | | AGTACAAGAAACAATTCAGATACGAGTCCCAGCTCCA |
| | | | GATGGTGCAGGTTACCGGGTCTTCAGACAACGAGTAT |
| | | | TTCTACGTGGATTTTCGCGAATACGAGTATGATCTGAA |
| | | | GTGGGAGTTCCCTAGAGAGAACCTGGAATTTGGCAAA |
| | | | GTGCTGGGATCCGGTGCCTTTGGCAAGGTGATGAACG |
| | | | CAACAGCCTACGGTATCAGCAAGACTGGCGTGTCTAT |
| | | | CCAGGTGGCAGTGAAAATGCTTAAGGAAAAGGCTGAT |
| | | | TCCTCTGAGCGCGAGGCCTTGATGTCCGAGTTGAAGAT |
| | | | GATGACACAGCTGGGCTCTCACGAGAACATCGTGAAC |
| | | | CTCCTGGGGGCCTGCACCCTGTCCGGGCCCATCTATCT |
| | | | CATCTTCGAGTACTGTTGCTATGGCGACCTCCTGAACT |
| | | | ATCTCAGGAGTAAGAGGGAGAAATTCCACAGGACCTG |
| | | | GACCGAGATCTTCAAAGAACATAACTTCTCCTTTTACC |
| | | | CCACCTTCCAGTCTCACCCTAACTCTTCCATGCCAGGA |
| | | | AGCCGTGAAGTGCAGATTCACCCCGACTCCGACCAGA |
| | | | TCTCCGGTTTGCACGGCAACTCCTTCCACAGCGAGGAT |
| | | | GAAATCGAGTACGAGAACCAGAAGCGTCTGGAGGAA |
| | | | GAGGAAGACCTGAACGTGCTGACATTTGAGGACTTGC |
| | | | TGTGCTTCGCCTATCAAGTGGCAAAGGGCATGGAATTT |
| | | | CTGGAATTCAAGTCTTGTGTCCACCGCGACCTTGCTGC |
| | | | GCGCAACGTACTGGTCACCCATGGTAAAGTTGTGAAG |
| | | | ATCTGTGATTTCGGCCTGGCCCGCGACATCATGTCTGA |
| | | | TTCCAACTATGTCGTGAGGGGCAATGCCCGTTTGCCTG |
| | | | TTAAGTGGATGGCTCCGGAGTCTCTGTTTGAGGGCATC |
| | | | TACACCATCAAGAGTGACGTATGGAGCTATGGTATTCT |
| | | | GCTTTGGGAGATCTTCAGTCTGGGCGTCAACCCTTATC |
| | | | CAGGTATTCCCGTGGACGCGAACTTTTACAAGCTGATT |
| | | | CAGAACGGCTTCAAAATGGACCAGCCATTCTACGCGA |
| | | | CCGAGGAAATCTACATCATTATGCAGTCTTGTTGGGCC |
| | | | TTCGATTCCCGCAAGCGGCCATCTTTTCCAAATCTCAC |
| | | | CTCCTTTCTCGGTTGTCAGCTGGCGGACGCTGAGGAAG |
| | | | CAATGTACCAGAACGTTGACGGTCGCGTGTCCGAATG |
| | | | TCCGCACACCTACCAAAACCGCCGTCCATTTTCCCGCG |
| | | | AGATGGACCTGGGGTTGCTGTCCCCGCAGGCACAAGT |
| | | | GGAAGACAGC (SEQ ID NO: 114) |
| TREM1 | TREM1 | 54210 | ATGCGCAAAACTAGACTGTGGGGTCTCCTGTGGATGTT |
| | | | GTTCGTCTCCGAGCTGAGGGCAGCCACTAAGCTGACT |
| | | | GAGGAAAAGTATGAACTGAAGGAAGGCCAAACTTTGG |
| | | | ATGTGAAGTGCGATTACACCCTCGAGAAGTTTGCCTCT |
| | | | AGCCAGAAGGCGTGGCAGATCATTCGCGATGGTGAGA |
| | | | TGCCTAAGACTCTGGCCTGTACAGAGCGCCCCAGCAA |
| | | | AAATTCCCATCCGGTGCAGGTGGGGAGAATTATCCTG |
| | | | GAGGATTACCACGATCACGGCCTTCTGCGTGTTCGCAT |
| | | | GGTGAACCTGCAAGTTGAGGACAGCGGCTTGTACCAG |
| | | | TGTGTTATCTACCAGCCTCCGAAGGAGCCACATATGCT |
| | | | GTTTGACCGCATCCGCCTGGTGGTAACTAAGGGCTTCT |
| | | | CCGGCACCCCTGGTTCAAATGAAAACTCCACCCAGAA |
| | | | CGTGTACAAGATCCCTCCAACAACCACAAAGGCCCTC |
| | | | TGCCCACTGTACACGAGCCCGCGCACTGTAACCCAGG |
| | | | CTCCTCCGAAGAGTACTGCAGACGTCTCTACGCCAGAT |
| | | | TCTGAGATCAACCTTACCAACGTGACAGACATCATTCG |
| | | | CGTTCCTGTGTTCAATATCGTCATCTTGCTGGCCGGTG |
| | | | GCTTCCTGTCTAAGAGCCTGGTGTTCAGTGTGCTCTTC |
| | | | GCAGTCACACTGCGCAGTTTTGTTCCT (SEQ ID NO: |
| | | | 115) |
| TREM2 | TREM2 | 54209 | ATGGAGCCGTTGCGTCTGCTCATCCTCCTGTTTGTCAC |
| | | | CGAGCTCAGCGGCGCGCACAACACTACCGTGTTCCAG |
| | | | GGGGTGGCTGGCCAGAGCCTCCAAGTGTCTTGTCCCTA |
| | | | CGACAGCATGAAGCATTGGGGACGCAGAAAGGCGTGG |
| | | | TGCCGCCAGCTGGGAGAGAAAGGCCCCTGCCAGCGCG |
| | | | TGGTCTCCACACACAATCTGTGGCTCCTGTCTTTCCTG |
| | | | CGTCGCTGGAATGGTAGCACCGCCATCACCGATGACA |
| | | | CTCTTGGAGGCACCTTGACTATTACTCTGAGAAACCTG |
| | | | CAGCCACACGACGCTGGGTTGTACCAATGCCAGAGCC |
| | | | TGCATGGATCTGAGGCCGACACCCTGCGCAAGGTACT |
| | | | GGTAGAGGTGTTGGCTGATCCGCTTGACCACAGAGAC |
| | | | GCCGGCGACCTGTGGTTCCCCGGCGAATCTGAGAGCTT |
| | | | TGAGGACGCCCATGTTGAGCACAGTATTTCCCGCAGCC |
| | | | TTCTGGAAGGCGAGATCCCGTTCCCGCCCACATCCATC |
| | | | CTGCTCCTGTTGGCCTGTATCTTCTTGATTAAGATCCTG |
| | | | GCCGCTAGCGCTCTTTGGGCCGCTGCCTGGCACGGCCA |
| | | | GAAGCCTGGGACCCACCCACCCTCCGAACTGGACTGT |
| | | | GGGCATGACCCAGGTTACCAACTGCAGACTTTGCCCG |
| | | | GCCTGCGCGACACC (SEQ ID NO: 116) |
| IFNλ | IFNLR1 | 163702 | AAAACCCTGATGGGCAACCCCTGGTTTCAGCGGGCCA |
| | | | AGATGCCTAGAGCACTGGACTTTAGCGGCCACACACA |
| | | | CCCCGTGGCCACCTTTCAACCTAGCAGACCTGAGAGC |
| | | | GTGAACGACCTGTTCCTGTGTCCTCAGAAAGAACTGAC |
| | | | CAGAGGCGTGCGGCCCACACCTAGAGTCAGAGCACCA |
| | | | GCTACACAGCAGACCAGATGGAAGAAGGACCTGGCCG |
| | | | AGGACGAGGAAGAGGAGGACGAAGAGGATACCGAGG |
| | | | ACGGCGTGTCCTTCCAGCCTTATATCGAGCCTCCTAGC |
| | | | TTCCTGGGCCAAGAGCATCAGGCCCCTGGACATTCTGA |
| | | | AGCCGGCGGAGTTGATAGCGGCAGACCAAGAGCACCT |
| | | | CTGGTGCCTAGCGAAGGATCTAGCGCCTGGGACAGCA |
| | | | GCGATAGAAGCTGGGCCAGCACAGTGGATAGCAGCTG |
| | | | GGATAGAGCCGGCAGCTCTGGATATCTGGCCGAGAAA |
| | | | GGACCAGGACAAGGCCCTGGCGGAGATGGCCACCAAG |
| | | | AATCTCTTCCACCTCCTGAGTTCAGCAAGGACAGCGGC |
| | | | TTTCTCGAGGAACTGCCCGAGGACAATCTGTCCAGCTG |
| | | | GGCTACATGGGGCACACTGCCTCCAGAGCCTAATCTG |
| | | | GTTCCTGGCGGACCTCCTGTGTCTCTGCAGACCCTGAC |
| | | | CTTTTGCTGGGAGAGCAGCCCCGAGGAAGAAGAAGAG |
| | | | GCCAGAGAGTCCGAGATCGAGGATTCCGATGCCGGAT |
| | | | CTTGGGGAGCCGAGAGCACACAGAGAACCGAGGATA |
| | | | GAGGCAGAACCCTGGGCCACTACATGGCCAGA (SEQ |
| | | | ID NO: 117) |
| IFNα/β/ω/ε/κ | IFNAR2 | 3455 | AAGTGGATCGGCTACATCTGCCTGAGAAACAGCCTGC |
| | | | CTAAGGTGCTGAACTTCCACAACTTTCTGGCCTGGCCT |
| | | | TTTCCTAACCTGCCTCCTCTGGAAGCCATGGACATGGT |
| | | | GGAAGTGATCTACATCAACCGGAAGAAGAAAGTCTGG |
| | | | GACTACAACTACGACGACGAGAGCGACAGCGACACAG |
| | | | AGGCCGCTCCTAGAACATCTGGCGGCGGATATACAAT |
| | | | GCACGGCCTGACCGTTAGACCTCTCGGACAGGCCTCTG |
| | | | CCACAAGCACAGAGAGCCAGCTGATCGACCCTGAGAG |
| | | | CGAGGAAGAACCTGACCTGCCTGAGGTGGACGTGGAA |
| | | | CTGCCTACCATGCCTAAGGACAGCCCTCAGCAGCTGG |
| | | | AACTGCTCTCTGGCCCTTGCGAGAGAAGAAAGAGCCC |
| | | | TCTGCAGGACCCATTTCCTGAAGAGGACTACAGCTCCA |
| | | | CCGAAGGCTCTGGCGGCAGAATCACCTTCAACGTGGA |
| | | | CCTGAACAGCGTGTTCCTGAGAGTGCTGGACGACGAG |
| | | | GATAGCGACGACCTGGAAGCTCCTCTGATGCTGAGCA |
| | | | GCCACCTGGAAGAGATGGTGGACCCCGAGGATCCCGA |
| | | | CAACGTGCAGAGCAATCATCTGCTGGCTAGCGGCGAG |
| | | | GGCACCCAGCCTACATTTCCATCTCCAAGCAGCGAAG |
| | | | GCCTTTGGAGCGAGGATGCCCCTAGCGATCAGAGCGA |
| | | | TACCAGCGAGTCCGATGTGGACCTCGGCGACGGCTAT |
| | | | ATCATGCGG (SEQ ID NO: 118) |
| IFNγ | IFNGR1 | 3459 | TGCTTCTACATCAAGAAGATCAACCCGCTGAAAGAGA |
| | | | AGTCCATCATCCTGCCTAAGAGCCTGATCAGCGTCGTG |
| | | | CGCTCTGCCACACTGGAAACAAAGCCCGAGAGCAAAT |
| | | | ACGTGTCCCTGATCACCAGCTACCAGCCTTTCAGCCTG |
| | | | GAAAAAGAGGTCGTCTGCGAGGAACCTCTGAGCCCTG |
| | | | CTACAGTGCCTGGCATGCACACCGAGGACAATCCCGG |
| | | | AAAGGTGGAACACACAGAGGAACTGAGCAGCATCACC |
| | | | GAGGTGGTCACCACCGAAGAGAACATCCCCGATGTGG |
| | | | TGCCAGGCAGCCACCTGACACCTATCGAGAGAGAGTC |
| | | | TAGCAGCCCTCTGTCCAGCAATCAGAGCGAGCCTGGA |
| | | | TCTATCGCCCTGAACAGCTACCACAGCCGGAACTGCA |
| | | | GCGAGAGCGACCACAGCAGAAACGGCTTCGACACCGA |
| | | | CAGCAGCTGCCTGGAATCTCACAGCAGCCTGAGCGAC |
| | | | AGCGAGTTCCCTCCAAACAACAAGGGCGAGATCAAGA |
| | | | CCGAGGGCCAAGAGCTGATCACCGTGATTAAGGCCCC |
| | | | TACCAGCTTCGGCTACGACAAGCCTCATGTGCTGGTCG |
| | | | ATCTGCTGGTGGACGACAGCGGCAAAGAGTCTCTGAT |
| | | | CGGCTACAGACCCACCGAGGATAGCAAAGAGTTCAGC |
| | | | (SEQ ID NO: 119) |
| N/A | STAT1 minimal | 3459 | CCTACCAGCTTCGGCTACGACAAACCCCATGTGCTG |
| | binding (from | | (SEQ ID NO: 120) |
| | IFNGR1) | | |
| IL10 | IL10Ra | 3587 | CAGCTGTATGTGCGGCGGAGAAAGAAACTGCCCAGCG |
| | | | TCCTGCTGTTCAAGAAGCCCTCTCCATTCATCTTCATC |
| | | | AGCCAGCGGCCTTCTCCAGAGACACAGGACACCATCC |
| | | | ATCCTCTGGACGAAGAGGCCTTCCTGAAGGTGTCCCCT |
| | | | GAGCTGAAGAACCTGGATCTGCACGGCAGCACCGATA |
| | | | GCGGCTTTGGCAGCACAAAGCCTAGCCTGCAGACCGA |
| | | | GGAACCCCAGTTCCTGCTGCCTGATCCTCATCCTCAGG |
| | | | CCGATAGAACCCTGGGCAACAGAGAACCTCCTGTGCT |
| | | | GGGCGATAGCTGTAGCAGCGGCAGCAGCAATAGCACC |
| | | | GACTCCGGCATCTGTCTGCAAGAGCCTTCTCTGAGCCC |
| | | | AAGCACAGGCCCTACATGGGAGCAGCAAGTGGGCAGC |
| | | | AATTCCAGAGGCCAGGATGACAGCGGAATCGACCTGG |
| | | | TGCAGAACTCTGAAGGCAGAGCCGGCGATACACAAGG |
| | | | CGGATCTGCTCTGGGACACCACTCTCCACCTGAGCCTG |
| | | | AAGTTCCCGGCGAAGAGGATCCTGCCGCTGTGGCATTT |
| | | | CAGGGCTACCTGAGACAGACCAGATGCGCCGAGGAAA |
| | | | AGGCCACCAAGACCGGCTGTCTGGAAGAGGAATCCCC |
| | | | TCTGACAGATGGACTGGGCCCCAAGTTCGGCAGATGC |
| | | | CTGGTTGATGAAGCCGGACTGCATCCTCCTGCTCTGGC |
| | | | CAAGGGATACCTGAAGCAGGACCCTCTGGAAATGACC |
| | | | CTGGCTTCTTCTGGCGCCCCTACCGGACAGTGGAATCA |
| | | | GCCTACAGAGGAATGGTCCCTGCTGGCCCTGAGCAGC |
| | | | TGTAGCGATCTGGGCATCAGCGATTGGAGCTTCGCCCA |
| | | | CGATCTGGCCCCACTGGGATGTGTTGCTGCACCTGGTG |
| | | | GACTGCTGGGCTCCTTCAATAGCGACCTGGTCACCCTG |
| | | | CCACTGATCAGCAGTCTGCAGAGCAGCGAG (SEQ ID |
| | | | NO: 121) |
| N/A | STAT3 minimal | 3587 | CTGGCCAAGGGCTACCTGAAGCAGGACCCTCTG (SEQ |
| | binding (from | | ID NO: 122) |
| | IL10Ra) | | |
| Tyro3 | Tyro3 | 7301 | ATGGCCCTGAGAAGATCCATGGGCAGACCTGGACTTC |
| | | | CACCTCTGCCTTTGCCTCCACCTCCTAGACTGGGACTG |
| | | | CTGCTGGCTGCTCTTGCTTCTCTGCTGCTGCCTGAATCT |
| | | | GCCGCCGCTGGACTGAAACTGATGGGAGCCCCTGTGA |
| | | | AGCTGACCGTGTCTCAGGGACAGCCCGTGAAACTGAA |
| | | | CTGCAGCGTGGAAGGCATGGAAGAACCCGACATCCAG |
| | | | TGGGTCAAAGATGGCGCCGTGGTGCAGAACCTGGACC |
| | | | AGCTGTATATCCCCGTGTCCGAGCAGCACTGGATCGGC |
| | | | TTTCTGAGCCTGAAGTCCGTGGAAAGAAGCGACGCCG |
| | | | GCAGATACTGGTGCCAGGTTGAAGATGGCGGCGAAAC |
| | | | CGAGATCAGCCAGCCTGTGTGGCTGACAGTGGAAGGC |
| | | | GTGCCATTCTTCACCGTGGAACCCAAGGATCTGGCCGT |
| | | | GCCTCCTAACGCTCCATTCCAGCTGTCTTGCGAAGCCG |
| | | | TGGGACCTCCTGAGCCTGTGACAATAGTTTGGTGGCGG |
| | | | GGCACCACAAAGATCGGCGGACCTGCTCCTTCTCCTTC |
| | | | CGTGCTGAATGTGACCGGCGTGACCCAGAGCACCATG |
| | | | TTTAGCTGCGAGGCCCACAACCTGAAAGGCCTGGCCT |
| | | | CTAGCAGAACCGCCACCGTGCATCTTCAAGCCTTGCCT |
| | | | GCCGCTCCTTTCAACATCACCGTGACCAAGCTGAGCAG |
| | | | CAGCAATGCCTCTGTGGCTTGGATGCCTGGCGCTGATG |
| | | | GTAGAGCACTGCTGCAGAGCTGTACCGTGCAAGTGAC |
| | | | ACAAGCTCCAGGCGGCTGGGAAGTGCTGGCTGTGGTT |
| | | | GTTCCTGTGCCTCCTTTCACCTGTCTGCTGAGAGATCT |
| | | | GGTGCCTGCCACCAACTACAGCCTGAGAGTCAGATGC |
| | | | GCCAACGCTCTGGGCCCTTCTCCATATGCTGATTGGGT |
| | | | GCCCTTCCAGACCAAAGGACTGGCCCCTGCTTCTGCCC |
| | | | CTCAGAATCTGCACGCCATCAGAACCGATAGCGGCCT |
| | | | GATCCTGGAATGGGAAGAAGTGATCCCCGAGGCTCCT |
| | | | CTGGAAGGACCTCTGGGACCTTACAAGCTGTCCTGGGT |
| | | | GCAAGACAACGGCACACAGGACGAACTGACCGTCGAG |
| | | | GGCACAAGAGCCAATCTGACAGGCTGGGACCCTCAGA |
| | | | AAGACCTGATCGTCAGAGTGTGCGTGTCCAACGCCGTT |
| | | | GGCTGTGGACCTTGGAGTCAGCCTCTGGTGGTGTCCTC |
| | | | TCACGATAGAGCTGGACAGCAGGGCCCTCCTCACAGC |
| | | | AGAACATCTTGGGTGCCAGTGGTGCTGGGCGTGCTGA |
| | | | CAGCTCTTGTTACAGCTGCTGCCCTGGCTCTGATCCTG |
| | | | CTGCGGAAGCGGAGAAAAGAAACCAGATTCGGACAG |
| | | | GCCTTCGACAGCGTGATGGCCAGAGGCGAACCTGCCG |
| | | | TGCACTTTAGAGCCGCCAGAAGCTTCAACAGAGAGCG |
| | | | GCCCGAGAGAATCGAGGCCACACTGGATTCTCTGGGC |
| | | | ATCAGCGACGAGCTGAAAGAAAAGCTGGAAGATGTGC |
| | | | TGATCCCGGAACAGCAGTTCACCCTGGGCAGAATGCT |
| | | | CGGCAAGGGCGAGTTTGGATCTGTGCGGGAAGCCCAG |
| | | | CTCAAGCAAGAGGATGGCAGCTTCGTGAAGGTGGCCG |
| | | | TGAAGATGCTGAAGGCCGACATCATTGCCAGCAGCGA |
| | | | TATCGAAGAGTTCCTGCGCGAAGCCGCCTGCATGAAG |
| | | | GAATTCGATCACCCTCACGTGGCCAAGCTCGTGGGAG |
| | | | TGTCTCTGAGAAGCAGAGCCAAAGGCAGGCTGCCCAT |
| | | | TCCTATGGTCATCCTGCCTTTCATGAAGCACGGCGACC |
| | | | TGCACGCCTTTCTGCTGGCCTCCAGAATCGGCGAGAAC |
| | | | CCCTTCAATCTGCCTCTGCAGACCCTGATCCGGTTCAT |
| | | | GGTGGATATCGCCTGCGGCATGGAATACCTGAGCAGC |
| | | | CGGAACTTCATCCACCGCGATCTGGCTGCCAGAAACT |
| | | | GCATGCTGGCCGAGGATATGACCGTGTGCGTGGCCGA |
| | | | TTTTGGCCTGAGCCGGAAGATCTACAGCGGCGACTACT |
| | | | ACAGACAGGGCTGCGCCTCTAAGCTGCCCGTTAAGTG |
| | | | GCTGGCCCTGGAAAGCCTGGCCGACAATCTGTACACC |
| | | | GTGCAGTCTGACGTGTGGGCCTTTGGCGTGACCATGTG |
| | | | GGAGATCATGACCAGAGGCCAGACACCTTACGCCGGC |
| | | | ATCGAGAACGCCGAGATCTACAACTATCTGATCGGCG |
| | | | GCAACCGGCTGAAGCAGCCTCCTGAGTGTATGGAAGA |
| | | | TGTCTACGACCTGATGTACCAGTGTTGGAGCGCCGATC |
| | | | CTAAGCAGCGGCCTAGCTTTACCTGCCTGAGAATGGA |
| | | | ACTGGAAAACATCCTGGGCCAGCTGAGCGTCCTGAGC |
| | | | GCTTCTCAGGATCCCCTGTACATCAACATCGAGAGAGC |
| | | | CGAGGAACCCACAGCCGGCGGATCTCTTGAACTGCCT |
| | | | GGCAGGGATCAGCCTTATTCTGGTGCTGGCGACGGCTC |
| | | | TGGCATGGGAGCTGTTGGAGGAACCCCAAGCGACTGC |
| | | | CGGTACATTCTTACACCTGGCGGACTGGCCGAACAGC |
| | | | CTGGACAGGCTGAACACCAGCCAGAGAGCCCTCTGAA |
| | | | CGAGACACAACGTCTGCTGCTCCTCCAGCAGGGACTG |
| | | | CTCCCTCACTCTTCTTGT (SEQ ID NO: 123) |
| Axl | Axl | 558 | ATGGCTTGGAGATGCCCCAGAATGGGCAGAGTGCCTC |
| | | | TGGCTTGGTGTCTGGCTCTTTGTGGCTGGGCCTGTATG |
| | | | GCCCCTAGAGGAACACAGGCCGAGGAATCCCCATTCG |
| | | | TGGGCAACCCCGGAAATATCACAGGCGCCAGAGGACT |
| | | | GACCGGCACACTGAGATGTCAACTGCAGGTTCAGGGC |
| | | | GAGCCTCCTGAAGTGCACTGGCTGAGAGATGGCCAGA |
| | | | TCCTGGAACTGGCCGACAGCACACAGACACAGGTGCC |
| | | | ACTGGGAGAAGATGAGCAGGACGACTGGATCGTGGTG |
| | | | TCCCAGCTGAGAATCACCAGCCTGCAGCTGAGCGATA |
| | | | CCGGCCAGTATCAGTGCCTGGTGTTTCTGGGCCACCAG |
| | | | ACCTTCGTGTCTCAGCCTGGATATGTGGGCCTCGAGGG |
| | | | CCTGCCTTACTTCCTGGAAGAACCCGAGGATAGAACC |
| | | | GTGGCCGCCAACACACCCTTCAACCTGTCTTGTCAGGC |
| | | | CCAGGGACCTCCTGAGCCTGTTGATCTGCTGTGGCTGC |
| | | | AGGATGCCGTGCCTCTTGCTACAGCTCCTGGACACGGA |
| | | | CCTCAGAGAAGCCTGCATGTGCCTGGACTGAACAAGA |
| | | | CCAGCAGCTTCAGCTGCGAAGCCCACAATGCCAAGGG |
| | | | CGTGACCACAAGCAGAACCGCCACAATCACAGTGCTG |
| | | | CCCCAGCAGCCTAGAAACCTGCACCTGGTTTCCAGAC |
| | | | AGCCCACCGAGCTGGAAGTGGCTTGGACACCTGGCCT |
| | | | GAGCGGCATCTATCCTCTGACACACTGTACACTGCAGG |
| | | | CCGTGCTGAGCGACGATGGCATGGGAATTCAAGCCGG |
| | | | CGAGCCTGATCCTCCTGAGGAACCACTTACAAGCCAG |
| | | | GCCTCTGTGCCTCCTCATCAGCTGAGACTGGGATCTCT |
| | | | GCACCCTCACACACCCTACCACATCAGAGTGGCCTGC |
| | | | ACAAGCTCTCAGGGCCCTAGCTCTTGGACCCACTGGCT |
| | | | GCCTGTGGAAACACCTGAAGGCGTTCCACTGGGCCCT |
| | | | CCAGAGAATATCAGCGCCACCAGAAATGGCTCCCAGG |
| | | | CCTTCGTGCATTGGCAAGAGCCTAGAGCACCTCTGCAG |
| | | | GGCACACTGCTGGGATACAGACTGGCCTACCAGGGCC |
| | | | AAGATACCCCTGAGGTGCTGATGGACATCGGCCTGAG |
| | | | ACAAGAAGTGACACTGGAACTGCAAGGCGACGGCAGC |
| | | | GTGTCCAATCTGACAGTGTGTGTGGCCGCCTATACAGC |
| | | | CGCTGGCGACGGACCTTGGTCACTGCCTGTTCCTCTGG |
| | | | AAGCTTGGAGGCCTGGACAGGCACAGCCTGTGCATCA |
| | | | GCTGGTCAAAGAGCCTTCCACTCCTGCCTTCAGCTGGC |
| | | | CTTGGTGGTACGTTCTGCTGGGAGCTGTGGTGGCTGCT |
| | | | GCCTGCGTTCTGATTCTGGCCCTGTTCCTGGTGCACCG |
| | | | GCGGAAGAAAGAGACAAGATACGGCGAGGTGTTCGA |
| | | | GCCCACAGTGGAAAGGGGAGAGCTGGTCGTCCGGTAC |
| | | | AGAGTGCGGAAGTCCTACAGCAGACGGACAACCGAGG |
| | | | CCACACTGAACAGCCTGGGAATCAGCGAGGAACTGAA |
| | | | AGAAAAGCTGCGGGACGTGATGGTGGATCGGCACAAA |
| | | | GTGGCCCTGGGCAAGACACTCGGAGAGGGCGAATTTG |
| | | | GCGCCGTGATGGAAGGACAGCTGAACCAGGACGACAG |
| | | | CATCCTGAAGGTGGCCGTGAAAACCATGAAGATCGCC |
| | | | ATCTGCACCAGAAGCGAACTCGAGGACTTTCTGAGCG |
| | | | AGGCCGTGTGCATGAAGGAATTCGATCACCCCAACGT |
| | | | GATGCGGCTGATCGGAGTGTGTTTTCAGGGCAGCGAG |
| | | | CGCGAGAGCTTTCCTGCTCCTGTGGTCATCCTGCCTTT |
| | | | CATGAAGCACGGCGACCTGCACAGCTTTCTGCTGTACT |
| | | | CCAGACTGGGCGACCAGCCTGTGTACCTGCCTACACA |
| | | | GATGCTGGTCAAGTTCATGGCCGATATCGCCTCCGGCA |
| | | | TGGAATACCTGAGCACCAAGCGGTTCATCCACAGAGA |
| | | | TCTGGCCGCCAGAAACTGCATGCTGAACGAGAACATG |
| | | | AGCGTGTGCGTGGCCGATTTCGGCCTGTCCAAGAAGA |
| | | | TCTACAACGGCGACTACTACCGGCAGGGCAGAATCGC |
| | | | CAAGATGCCCGTGAAGTGGATCGCCATTGAGAGCCTG |
| | | | GCCGATAGAGTGTACACCAGCAAGAGCGACGTGTGGT |
| | | | CCTTCGGCGTGACAATGTGGGAGATCGCCACCAGGGG |
| | | | ACAGACCCCTTATCCTGGCGTGGAAAACAGCGAGATC |
| | | | TACGACTACCTGAGACAGGGCAACAGACTGAAGCAGC |
| | | | CCGCCGATTGTCTGGATGGACTGTACGCCCTGATGAGC |
| | | | CGGTGTTGGGAGCTGAACCCTCAGGACAGACCTAGCT |
| | | | TTACCGAGCTGAGAGAGGACCTGGAAAACACCCTGAA |
| | | | AGCCCTGCCTCCAGCTCAAGAGCCCGACGAGATCCTG |
| | | | TACGTGAACATGGATGAAGGCGGCGGATACCCTGAAC |
| | | | CACCTGGTGCTGCTGGCGGAGCTGATCCACCTACACA |
| | | | GCCCGATCCTAAGGATAGCTGCTCCTGCCTGACAGCCG |
| | | | CCGAAGTTCATCCAGCCGGCAGATACGTGCTGTGCCCT |
| | | | AGCACAACACCCTCTCCAGCACAGCCAGCCGACAGAG |
| | | | GATCTCCAGCTGCTCCAGGACAAGAAGATGGCGCT |
| | | | (SEQ ID NO: 124) |
| MerTK | MerTK | 10461 | ATGGGACCTGCTCCTCTGCCTCTGCTGCTGGGACTGTT |
| | | | TCTGCCTGCTCTTTGGCGGAGAGCCATCACCGAGGCCA |
| | | | GAGAGGAAGCCAAGCCTTATCCTCTGTTCCCCGGACCT |
| | | | TTTCCAGGCAGCCTGCAGACCGATCACACCCCTTTGCT |
| | | | GTCTCTGCCTCACGCCTCTGGCTATCAGCCCGCTCTGA |
| | | | TGTTCAGCCCCACACAGCCAGGCAGACCTCACACAGG |
| | | | CAATGTGGCCATTCCTCAAGTGACCAGCGTGGAAAGC |
| | | | AAGCCCTTGCCTCCTCTGGCCTTCAAGCACACAGTGGG |
| | | | CCACATCATCCTGAGCGAGCACAAGGGCGTGAAGTTC |
| | | | AACTGCAGCATCAGCGTGCCCAACATCTACCAGGACA |
| | | | CCACCATCAGCTGGTGGAAGGACGGCAAAGAACTGCT |
| | | | GGGAGCCCACCACGCCATCACACAGTTCTACCCCGAC |
| | | | GATGAAGTGACCGCCATCATTGCCAGCTTCAGCATCAC |
| | | | CAGCGTGCAGAGAAGCGACAACGGCAGCTACATCTGC |
| | | | AAGATGAAGATCAACAACGAGGAAATCGTCAGCGACC |
| | | | CCATCTACATCGAGGTGCAGGGCCTGCCTCACTTCACC |
| | | | AAGCAGCCCGAGAGCATGAACGTGACCAGAAACACCG |
| | | | CCTTCAACCTGACCTGTCAGGCCGTGGGACCTCCTGAG |
| | | | CCTGTGAACATCTTCTGGGTGCAGAACAGCTCCAGAGT |
| | | | GAACGAGCAGCCTGAGAAGTCCCCTAGCGTGCTGACA |
| | | | GTGCCTGGACTGACAGAGATGGCCGTGTTTTCTTGCGA |
| | | | GGCCCACAACGACAAGGGCCTGACCGTGTCTAAGGGC |
| | | | GTGCAGATCAATATCAAGGCCATTCCATCTCCACCTAC |
| | | | CGAGGTGTCCATCCGGAATAGCACAGCCCACTCCATC |
| | | | CTGATCTCTTGGGTGCCCGGCTTCGATGGCTACAGCCC |
| | | | CTTCAGAAACTGCTCCATCCAAGTGAAAGAGGCCGAT |
| | | | CCTCTGAGCAACGGCTCCGTGATGATCTTCAACACAAG |
| | | | CGCCCTGCCACACCTGTACCAGATCAAACAGCTGCAG |
| | | | GCTCTGGCCAACTACTCCATCGGCGTGTCCTGCATGAA |
| | | | CGAGATCGGCTGGAGTGCCGTGTCTCCTTGGATCCTGG |
| | | | CCAGCACAACTGAAGGCGCTCCATCTGTGGCCCCTCTG |
| | | | AATGTGACCGTGTTCCTGAACGAGAGCAGCGACAATG |
| | | | TGGACATCCGGTGGATGAAGCCACCTACAAAGCAGCA |
| | | | GGACGGCGAACTCGTGGGCTACAGAATCTCTCACGTG |
| | | | TGGCAGTCTGCCGGCATCTCCAAAGAACTCCTCGAAG |
| | | | AAGTGGGCCAGAACGGCAGCAGAGCCAGGATCTCTGT |
| | | | GCAGGTCCACAACGCCACATGCACAGTGCGGATTGCC |
| | | | GCTGTGACAAGAGGCGGAGTGGGCCCTTTTAGCGACC |
| | | | CCGTGAAGATCTTTATCCCCGCTCACGGCTGGGTCGAC |
| | | | TACGCCCCATCTTCTACACCAGCTCCAGGCAACGCTGA |
| | | | CCCCGTGCTGATCATCTTCGGCTGCTTTTGCGGCTTTAT |
| | | | CCTGATCGGCCTGATCCTGTACATCAGCCTGGCCATCA |
| | | | GAAAGCGGGTGCAAGAGACAAAGTTCGGCAACGCCTT |
| | | | CACCGAAGAGGACAGCGAGCTGGTGGTCAACTATATC |
| | | | GCCAAGAAGTCCTTCTGCAGACGGGCCATCGAGCTGA |
| | | | CACTGCACAGTCTGGGAGTGTCCGAGGAACTGCAGAA |
| | | | CAAGCTGGAAGATGTGGTCATCGACCGGAACCTGCTG |
| | | | ATCCTGGGCAAGATTCTCGGCGAGGGCGAGTTTGGCT |
| | | | CTGTGATGGAAGGCAACCTGAAGCAAGAGGACGGCAC |
| | | | CTCTCTGAAGGTGGCCGTGAAAACCATGAAGCTGGAC |
| | | | AACAGCAGCCAGCGCGAGATCGAAGAGTTTCTGTCTG |
| | | | AGGCCGCCTGTATGAAGGATTTCTCTCACCCCAACGTG |
| | | | ATCCGGCTGCTGGGCGTGTGTATCGAGATGTCTAGCCA |
| | | | GGGCATCCCCAAGCCTATGGTCATCCTGCCTTTCATGA |
| | | | AGTACGGCGATCTGCACACCTACCTGCTGTACTCCAGA |
| | | | CTGGAAACAGGCCCCAAGCACATCCCTCTGCAGACCC |
| | | | TGCTGAAGTTCATGGTGGATATCGCCCTCGGCATGGAA |
| | | | TACCTGAGCAACCGGAACTTCCTGCACCGCGATCTGGC |
| | | | CGCCAGAAATTGCATGCTGAGGGACGACATGACCGTG |
| | | | TGCGTGGCCGATTTTGGCCTGAGCAAGAAGATCTACA |
| | | | GCGGCGACTACTACCGGCAGGGCAGAATTGCCAAGAT |
| | | | GCCCGTGAAGTGGATCGCCATCGAGAGCCTGGCCGAC |
| | | | AGAGTGTACACCAGCAAGTCTGACGTGTGGGCCTTCG |
| | | | GCGTGACCATGTGGGAGATTGCCACCAGAGGCATGAC |
| | | | CCCTTATCCTGGCGTCCAGAACCACGAGATGTACGATT |
| | | | ACCTGCTGCACGGCCACAGACTGAAGCAGCCAGAGGA |
| | | | TTGCCTGGACGAGCTGTACGAGATCATGTACTCTTGCT |
| | | | GGCGGACCGATCCACTGGACAGACCTACATTCTCCGT |
| | | | GCTGCGGCTGCAGCTGGAAAAACTGCTGGAAAGCCTG |
| | | | CCTGACGTGCGGAACCAGGCCGATGTGATCTACGTGA |
| | | | ACACCCAGCTGCTGGAATCCAGCGAAGGACTGGCCCA |
| | | | GGGATCTACACTGGCTCCTCTGGACCTGAACATCGACC |
| | | | CCGACAGCATTATCGCCAGCTGCACACCAAGAGCCGC |
| | | | CATCAGCGTTGTGACAGCCGAGGTGCACGATAGCAAG |
| | | | CCTCACGAAGGCCGGTACATCCTGAATGGCGGAAGCG |
| | | | AGGAATGGGAAGATCTGACCAGCGCTCCTTCTGCCGC |
| | | | CGTGACCGCCGAGAAAAATTCTGTGCTGCCTGGCGAG |
| | | | CGGCTCGTGCGAAACGGTGTCTCTTGGAGCCACAGCTC |
| | | | CATGCTGCCTCTGGGAAGCTCTCTGCCTGATGAGCTGC |
| | | | TGTTCGCCGACGATTCTAGCGAGGGATCCGAGGTGTTG |
| | | | ATG (SEQ ID NO: 125) |

TABLE 6
Exemplary Amino Acid and Nucleotide Sequences for Membrane-Tethered IFNβ
and STAT3/1 chimera

| Name | Amino Acid Sequence | Nucleotide Sequence |
|---|---|---|
| IFNβ | MTNKCLLQIALLLCFSTTALSMSYN | ATGAGCTACAACCTGCTGGGCTTCCTGCA |
| | LLGFLQRSSNFQCQKLLWQLNGRL | GCGGAGCAGCAACTTCCAGTGCCAGAAA |
| | EYCLKDRMNFDIPEEIKQLQQFQKE | CTGCTGTGGCAGCTGAACGGCCGGCTGGA |
| | DAALTIYEMLQNIFAIFRQDSSSTG | ATACTGCCTGAAGGACCGGATGAACTTCG |
| | WNETIVENLLANVYHQINHLKTVL | ACATCCCCGAGGAAATCAAGCAGCTGCA |
| | EEKLEKEDFTRGKLMSSLHLKRYY | GCAGTTCCAGAAAGAGGACGCCGCTCTG |
| | GRILHYLKAKEYSHCAWTIVRVEIL | ACCATCTACGAGATGCTGCAGAACATCTT |
| | RNFYFINRLTGYLRN (SEQ ID NO: | CGCCATCTTCCGGCAGGACAGCAGCTCCA |
| | 126) | CAGGCTGGAACGAGACAATCGTGGAAAA |
| | | TCTGCTGGCCAACGTGTACCACCAGATCA |
| | | ACCACCTGAAAACCGTGCTGGAAGAGAA |
| | | GCTGGAAAAAGAGGACTTCACCCGGGGC |
| | | AAGCTGATGAGCAGCCTGCACCTGAAGC |
| | | GGTACTACGGCAGAATCCTGCACTACCTG |
| | | AAGGCCAAAGAGTACAGCCACTGCGCCT |
| | | GGACCATCGTGCGCGTGGAAATCCTGCGG |
| | | AACTTCTACTTCATCAACCGGCTGACCGG |
| | | CTACCTGAGAAAT (SEQ ID NO: 137) |
| GPI anchor signal | EGISLLAQNTSWLLLLLLSLSLLQA | GAGGGAATTAGCCTGCTGGCCCAGAATAC |
| (from Thy1) | TDFMSL (SEQ ID NO: 127) | CAGCTGGCTGCTGCTGCTTCTGCTGAGCC |
| | | TGTCTCTGCTGCAAGCCACCGACTTCATG |
| | | AGCCTG (SEQ ID NO: 138) |
| (G4S)3 linker | GGGGSGGGGSGGGGS (SEQ ID | GGCGGCGGAGGATCTGGCGGAGGTGGAA |
| | NO: 128) | GCGGAGGCGGTGGATCT (SEQ ID NO: 139) |
| B7 TMD anchor | DNLLPSWAITLISVNGIFVICCLTYC | CTGCTGCCTAGCTGGGCCATCACACTGAT |
| | FAPRCRERRRNERLRRESVRPV | CTCCGTGAACGGCATCTTCGTGATCTGCT |
| | (SEQ ID NO: 129) | GTCTG (SEQ ID NO: 140) |
| CD28 spacer | IEVMYPPPYLDNEKSNGTIIHVKGK | ATCGAAGTGATGTACCCTCCACCTTACCT |
| | HLCPSPLFPGPSKPGS (SEQ ID NO: | GGACAACGAGAAGTCCAACGGCACCATC |
| | 130) | ATCCACGTGAAGGGCAAGCACCTGTGTCC |
| | | TTCTCCACTGTTCCCCGGACCTAGCAAGC |
| | | CTGGCTCT (SEQ ID NO: 141) |
| MMP3 substrate | RPKPVELWRK (SEQ ID NO: 131) | AGACCCAAGCCAGTGGAACTGTGGCGGA |
| | | AA (SEQ ID NO: 142) |
| IFNβ-GPI | MTNKCLLQIALLLCFSTTALSMSYN | ATGACCAACAAGTGCCTGCTGCAGATCGC |
| IFNβ/(G4S)3 | LLGFLQRSSNFQCQKLLWQLNGRL | CCTGCTGCTGTGCTTTAGCACAACAGCCC |
| linker/GPI | EYCLKDRMNFDIPEEIKQLQQFQKE | TGAGCATGAGCTACAACCTGCTGGGCTTC |
| anchor signal | DAALTIYEMLQNIFAIFRQDSSSTG | CTGCAGCGGAGCAGCAACTTCCAGTGCCA |
| | WNETIVENLLANVYHQINHLKTVL | GAAACTGCTGTGGCAGCTGAACGGCCGG |
| | EEKLEKEDFTRGKLMSSLHLKRYY | CTGGAATACTGCCTGAAGGACCGGATGA |
| | GRILHYLKAKEYSHCAWTIVRVEIL | ACTTCGACATCCCCGAGGAAATCAAGCAG |
| | RNFYFINRLTGYLRNGGGGSGGGG | CTGCAGCAGTTCCAGAAAGAGGACGCCG |
| | SGGGGSEGISLLAQNTSWLLLLLLS | CTCTGACCATCTACGAGATGCTGCAGAAC |
| | LSLLQATDFMSL (SEQ ID NO: 132) | ATCTTCGCCATCTTCCGGCAGGACAGCAG |
| | | CTCCACAGGCTGGAACGAGACAATCGTG |
| | | GAAAATCTGCTGGCCAACGTGTACCACCA |
| | | GATCAACCACCTGAAAACCGTGCTGGAA |
| | | GAGAAGCTGGAAAAAGAGGACTTCACCC |
| | | GGGGCAAGCTGATGAGCAGCCTGCACCT |
| | | GAAGCGGTACTACGGCAGAATCCTGCACT |
| | | ACCTGAAGGCCAAAGAGTACAGCCACTG |
| | | CGCCTGGACCATCGTGCGCGTGGAAATCC |
| | | TGCGGAACTTCTACTTCATCAACCGGCTG |
| | | ACCGGCTACCTGAGAAATGGCGGCGGAG |
| | | GATCTGGCGGAGGTGGAAGCGGAGGCGG |
| | | TGGATCTGAGGGAATTTCTCTGCTGGCTC |
| | | AGAACACCAGCTGGCTGCTGCTGCTTCTG |
| | | CTGTCTCTGAGTCTGCTGCAGGCCACCGA |
| | | CTTCATGAGCCTGTAA (SEQ ID NO: 143) |
| IFNβ-B7-TMD | MTNKCLLQIALLLCFSTTALSMSYN | ATGACCAACAAGTGCCTGCTGCAGATCGC |
| IFNβ/(G4S)3 | LLGFLQRSSNFQCQKLLWQLNGRL | CCTGCTGCTGTGCTTTAGCACAACAGCCC |
| linker/B7 TMD | EYCLKDRMNFDIPEEIKQLQQFQKE | TGAGCATGAGCTACAACCTGCTGGGCTTC |
| anchor | DAALTIYEMLQNIFAIFRQDSSSTG | CTGCAGCGGAGCAGCAACTTCCAGTGCCA |
| | WNETIVENLLANVYHQINHLKTVL | GAAACTGCTGTGGCAGCTGAACGGCCGG |
| | EEKLEKEDFTRGKLMSSLHLKRYY | CTGGAATACTGCCTGAAGGACCGGATGA |
| | GRILHYLKAKEYSHCAWTIVRVEIL | ACTTCGACATCCCCGAGGAAATCAAGCAG |
| | RNFYFINRLTGYLRNGGGGSGGGG | CTGCAGCAGTTCCAGAAAGAGGACGCCG |
| | SGGGGSDNLLPSWAITLISVNGIFVI | CTCTGACCATCTACGAGATGCTGCAGAAC |
| | CCLTYCFAPRCRERRRNERLRRESV | ATCTTCGCCATCTTCCGGCAGGACAGCAG |
| | RPV (SEQ ID NO: 133) | CTCCACAGGCTGGAACGAGACAATCGTG |
| | | GAAAATCTGCTGGCCAACGTGTACCACCA |
| | | GATCAACCACCTGAAAACCGTGCTGGAA |
| | | GAGAAGCTGGAAAAAGAGGACTTCACCC |
| | | GGGGCAAGCTGATGAGCAGCCTGCACCT |
| | | GAAGCGGTACTACGGCAGAATCCTGCACT |
| | | ACCTGAAGGCCAAAGAGTACAGCCACTG |
| | | CGCCTGGACCATCGTGCGCGTGGAAATCC |
| | | TGCGGAACTTCTACTTCATCAACCGGCTG |
| | | ACCGGCTACCTGAGAAATGGCGGCGGAG |
| | | GATCTGGCGGAGGTGGAAGCGGAGGCGG |
| | | TGGATCTGATAATCTGCTGCCCTCCTGGG |
| | | CCATCACACTGATCTCCGTGAACGGCATC |
| | | TTCGTGATCTGCTGTCTGACCTACTGCTTC |
| | | GCCCCTCGGTGCAGAGAGCGGAGAAGAA |
| | | ACGAACGGCTGCGGAGAGAATCTGTGCG |
| | | GCCTGTGTAA (SEQ ID NO: 144) |
| IFNβ-CD28-GPI | MTNKCLLQIALLLCFSTTALSMSYN | ATGACCAACAAGTGCCTGCTGCAGATCGC |
| IFNβ/CD28 | LLGFLQRSSNFQCQKLLWQLNGRL | CCTGCTGCTGTGCTTTAGCACAACAGCCC |
| spacer/GPI | EYCLKDRMNFDIPEEIKQLQQFQKE | TGAGCATGAGCTACAACCTGCTGGGCTTC |
| anchor signal | DAALTIYEMLQNIFAIFRQDSSSTG | CTGCAGCGGAGCAGCAACTTCCAGTGCCA |
| | WNETIVENLLANVYHQINHLKTVL | GAAACTGCTGTGGCAGCTGAACGGCCGG |
| | EEKLEKEDFTRGKLMSSLHLKRYY | CTGGAATACTGCCTGAAGGACCGGATGA |
| | GRILHYLKAKEYSHCAWTIVRVEIL | ACTTCGACATCCCCGAGGAAATCAAGCAG |
| | RNFYFINRLTGYLRNIEVMYPPPYL | CTGCAGCAGTTCCAGAAAGAGGACGCCG |
| | DNEKSNGTIIHVKGKHLCPSPLFPG | CTCTGACCATCTACGAGATGCTGCAGAAC |
| | PSKPGSEGISLLAQNTSWLLLLLLSL | ATCTTCGCCATCTTCCGGCAGGACAGCAG |
| | SLLQATDFMSL (SEQ ID NO: 134) | CTCCACAGGCTGGAACGAGACAATCGTG |
| | | GAAAATCTGCTGGCCAACGTGTACCACCA |
| | | GATCAACCACCTGAAAACCGTGCTGGAA |
| | | GAGAAGCTGGAAAAAGAGGACTTCACCC |
| | | GGGGCAAGCTGATGAGCAGCCTGCACCT |
| | | GAAGCGGTACTACGGCAGAATCCTGCACT |
| | | ACCTGAAGGCCAAAGAGTACAGCCACTG |
| | | CGCCTGGACCATCGTGCGCGTGGAAATCC |
| | | TGCGGAACTTCTACTTCATCAACCGGCTG |
| | | ACCGGCTACCTGAGAAACATCGAAGTGAT |
| | | GTACCCTCCACCTTACCTGGACAACGAGA |
| | | AGTCCAACGGCACCATCATCCACGTGAAG |
| | | GGCAAGCACCTGTGTCCTTCTCCACTGTT |
| | | CCCCGGACCTAGCAAGCCTGGCTCTGAGG |
| | | GAATTAGCCTGCTGGCCCAGAATACCAGC |
| | | TGGCTGCTGCTGCTTCTGCTGAGCCTGTCT |
| | | CTGCTGCAAGCCACCGACTTCATGAGCCT |
| | | GTGA (SEQ ID NO: 145) |
| IFNβ-MMP3-B7- | MTNKCLLQIALLLCFSTTALSMSYN | ATGACCAACAAGTGCCTGCTGCAGATCGC |
| TMD IFNβ/MMP3 | LLGFLQRSSNFQCQKLLWQLNGRL | CCTGCTGCTGTGCTTTAGCACAACAGCCC |
| substrate/ | EYCLKDRMNFDIPEEIKQLQQFQKE | TGAGCATGAGCTACAACCTGCTGGGCTTC |
| (G4S)3 linker/ | DAALTIYEMLQNIFAIFRQDSSSTG | CTGCAGCGGAGCAGCAACTTCCAGTGCCA |
| B7 TMD anchor | WNETIVENLLANVYHQINHLKTVL | GAAACTGCTGTGGCAGCTGAACGGCCGG |
| | EEKLEKEDFTRGKLMSSLHLKRYY | CTGGAATACTGCCTGAAGGACCGGATGA |
| | GRILHYLKAKEYSHCAWTIVRVEIL | ACTTCGACATCCCCGAGGAAATCAAGCAG |
| | RNFYFINRLTGYLRNRPKPVELWR | CTGCAGCAGTTCCAGAAAGAGGACGCCG |
| | KGGGGSGGGGSGGGGSDNLLPSW | CTCTGACCATCTACGAGATGCTGCAGAAC |
| | AITLISVNGIFVICCLTYCFAPRCRE | ATCTTCGCCATCTTCCGGCAGGACAGCAG |
| | RRRNERLRRESVRPV (SEQ ID NO: | CTCCACAGGCTGGAACGAGACAATCGTG |
| | 135) | GAAAATCTGCTGGCCAACGTGTACCACCA |
| | | GATCAACCACCTGAAAACCGTGCTGGAA |
| | | GAGAAGCTGGAAAAAGAGGACTTCACCC |
| | | GGGGCAAGCTGATGAGCAGCCTGCACCT |
| | | GAAGCGGTACTACGGCAGAATCCTGCACT |
| | | ACCTGAAGGCCAAAGAGTACAGCCACTG |
| | | CGCCTGGACCATCGTGCGCGTGGAAATCC |
| | | TGCGGAACTTCTACTTCATCAACCGGCTG |
| | | ACCGGCTACCTGAGAAACAGACCCAAGC |
| | | CAGTGGAACTGTGGCGGAAAGGTGGCGG |
| | | AGGATCTGGCGGAGGTGGAAGCGGCGGA |
| | | GGCGGATCTGATAATCTGCTGCCTAGCTG |
| | | GGCCATCACACTGATCTCCGTGAACGGCA |
| | | TCTTCGTGATCTGCTGTCTGACCTACTGCT |
| | | TCGCCCCTCGGTGCAGAGAGCGGAGAAG |
| | | AAACGAACGGCTGCGGAGAGAATCTGTG |
| | | CGGCCTGTGTAA (SEQ ID NO: 146) |
| STAT3/1 | MAQWNQLQQLDTRYLEQLHQLYS | ATGGCCCAGTGGAATCAGCTGCAGCAGCT |
| Chimera | DSFPMELRQFLAPWIESQDWAYAA | CGACACCAGATACCTCGAACAGCTCCACC |
| STAT3 (1-296)/ | SKESHATLVFHNLLGEIDQQYSRFL | AGCTGTACTCCGACAGCTTCCCTATGGAA |
| STATI DBD/ | QESNVLYQHNLRRIKQFLQSRYLE | CTGCGGCAGTTTCTGGCCCCTTGGATCGA |
| STAT3 (515-770) | KPMEIARIVARCLWEESRLLQTAAT | GTCTCAGGATTGGGCCTACGCCGCCAGCA |
| | AAQQGGQANHPTAAVVTEKQQML | AAGAGTCTCACGCCACACTGGTGTTCCAC |
| | EQHLQDVRKRVQDLEQKMKVVEN | AACCTGCTGGGCGAGATCGACCAGCAGT |
| | LQDDFDFNYKTLKSQGDMQDLNG | ACAGCCGGTTTCTGCAAGAGTCCAACGTG |
| | NNQSVTRQKMQQLEQMLTALDQM | CTGTACCAGCACAACCTGAGGCGGATCAA |
| | RRSIVSELAGLLSAMEYVQKTLTDE | GCAGTTCCTGCAGAGCAGATACCTGGAAA |
| | ELADWKRRQQIACIGGPPNICLDRL | AGCCCATGGAAATCGCCCGGATCGTGGCC |
| | ENWITSLAESQLQTRQQIKKLEELQ | AGATGTCTGTGGGAAGAAAGCAGACTGC |
| | QKVSYKGDPITKNKQVLWDRTFSL | TGCAGACCGCCGCTACAGCTGCTCAACAA |
| | FQQLIQSSFVVERQPCMPTHPQRPL | GGCGGACAGGCCAACCATCCTACAGCCG |
| | VLKTGVQFTVKLRLLVKLQELNYN | CCGTGGTTACAGAGAAGCAGCAGATGCT |
| | LKVKVLFDKDVNERNTVKGFRKF | GGAACAGCATCTCCAGGACGTGCGGAAG |
| | NILGTHTKVMNMEESTNGSLAAEF | AGAGTGCAGGACCTGGAACAGAAAATGA |
| | RHLQLKEQKNAGTRTNEGPLIVTE | AGGTGGTGGAAAACCTGCAGGACGACTT |
| | ELHSLSFETQLCQPGLVIDLETTSLP | CGACTTCAACTACAAGACCCTGAAGTCCC |
| | VVVISNVSQLPSGWASILWYNMLV | AGGGCGACATGCAGGATCTGAACGGCAA |
| | AEPRNLSFFLTPPCARWAQLSEVLS | CAACCAGAGCGTGACCCGGCAGAAGATG |
| | WQFSSTTKRGLSIEQLTTLAEKLLG | CAGCAACTCGAACAGATGCTGACAGCCCT |
| | PGVNYSGCQITWAKFCKENMAGK | GGACCAGATGAGGCGGAGCATTGTGTCTG |
| | GFSFWVWLDNIIDLVKKYILALWN | AACTGGCCGGACTGCTGAGCGCCATGGA |
| | EGYIMGFISKERERAILSTKPPGTFL | ATACGTGCAGAAAACCCTGACCGACGAG |
| | LRFSESSKEGGVTFTWVEKDISGKT | GAACTGGCTGACTGGAAAAGACGGCAGC |
| | QIQSVEPYTKQQLNNMSFAEIIMGY | AGATCGCCTGTATCGGCGGACCTCCTAAC |
| | KIMDATNILVSPLVYLYPDIPKEEA | ATCTGCCTGGACCGGCTGGAAAACTGGAT |
| | FGKYCRPESQEHPEADPGSAAPYL | CACAAGCCTGGCCGAAAGCCAGCTGCAG |
| | KTKFICVTPTTCSNTIDLPMSPRTLD | ACAAGACAGCAGATCAAGAAGCTGGAAG |
| | SLMQFGNNGEGAEPSAGGQFESLT | AACTGCAGCAGAAGGTGTCCTACAAGGG |
| | FDMELTSECATSPMDYKDDDDK | CGACCCCATCACCAAGAACAAACAGGTG |
| | (SEQ ID NO: 136) | CTGTGGGACAGAACCTTCAGCCTGTTTCA |
| | | GCAGCTGATCCAGAGCAGCTTCGTGGTGG |
| | | AACGGCAGCCCTGCATGCCTACACACCCT |
| | | CAAAGACCCCTGGTGCTGAAAACCGGCGT |
| | | GCAGTTCACCGTGAAGCTGCGGCTGCTGG |
| | | TCAAGCTGCAAGAGCTGAACTACAACCTG |
| | | AAAGTGAAGGTGCTGTTCGACAAGGACG |
| | | TGAACGAGCGGAACACCGTGAAAGGCTT |
| | | CCGCAAGTTCAACATCCTGGGCACCCACA |
| | | CCAAAGTGATGAACATGGAAGAGAGCAC |
| | | CAACGGCTCCCTGGCCGCCGAGTTTAGAC |
| | | ACCTCCAGCTGAAAGAGCAGAAGAACGC |
| | | CGGCACCAGGACCAATGAGGGACCTCTG |
| | | ATCGTGACAGAGGAACTGCACAGCCTGA |
| | | GCTTCGAAACCCAGCTGTGTCAGCCTGGC |
| | | CTGGTCATCGATCTGGAAACCACCTCTCT |
| | | GCCCGTGGTGGTCATCAGCAATGTGTCCC |
| | | AGCTGCCTTCTGGCTGGGCCAGCATCCTG |
| | | TGGTACAACATGCTGGTGGCCGAGCCTCG |
| | | GAACCTGTCCTTCTTTCTGACCCCTCCATG |
| | | TGCCAGATGGGCCCAGCTGTCTGAAGTCC |
| | | TGAGCTGGCAGTTTAGCAGCACCACCAAG |
| | | AGAGGCCTGAGCATCGAGCAGCTGACAA |
| | | CACTGGCCGAGAAGTTGCTTGGCCCTGGC |
| | | GTGAACTACAGCGGCTGTCAGATTACCTG |
| | | GGCCAAGTTCTGCAAAGAAAACATGGCC |
| | | GGCAAGGGCTTCAGCTTCTGGGTCTGGCT |
| | | GGACAACATCATCGACCTGGTCAAGAAGT |
| | | ACATTCTGGCCCTGTGGAACGAGGGCTAC |
| | | ATCATGGGCTTCATCTCCAAAGAGAGAGA |
| | | GCGGGCCATCCTGAGCACAAAGCCTCCAG |
| | | GCACCTTCCTGCTGAGATTCAGCGAGAGC |
| | | AGCAAAGAAGGCGGCGTCACCTTTACCTG |
| | | GGTCGAGAAGGATATCAGCGGCAAGACC |
| | | CAGATCCAGTCCGTGGAACCCTACACCAA |
| | | ACAACAGCTGAACAACATGAGCTTCGCCG |
| | | AGATCATCATGGGGTACAAGATCATGGAC |
| | | GCCACCAATATCCTGGTGTCCCCACTGGT |
| | | GTACCTGTATCCTGACATCCCCAAAGAGG |
| | | AAGCCTTCGGCAAGTACTGCAGACCCGAG |
| | | AGCCAAGAGCACCCTGAAGCCGATCCTG |
| | | GAAGCGCCGCTCCTTACCTGAAAACAAAG |
| | | TTCATCTGCGTGACCCCTACCACCTGTAG |
| | | CAACACAATCGACCTGCCTATGAGCCCCA |
| | | GAACACTGGACTCCCTGATGCAGTTCGGC |
| | | AACAATGGCGAAGGCGCCGAACCATCTG |
| | | CTGGCGGACAGTTTGAGTCCCTGACCTTC |
| | | GACATGGAACTGACCAGCGAGTGCGCCA |
| | | CCTCTCCTATGGACTACAAGGACGACGAC |
| | | GACAAGTGA (SEQ ID NO: 147) |

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes as described herein. The publications, websites and other reference materials referenced herein to describe the background of the invention and to provide additional detail regarding its practice are hereby incorporated by reference in their entireties.

1. A modified immune cell comprising a chimeric antigen receptor (CAR), wherein the CAR comprises:
(a) an extracellular domain;
(b) a transmembrane domain; and
(c) one or more intracellular moieties comprising:
(i) a first JAK binding domain or a portion thereof, and
(ii) a first STAT binding domain or a portion thereof;
wherein the modified immune cell is or comprises a stem cell, macrophage, monocyte, or dendritic cell.

2. The modified immune cell of claim 1, wherein the one or more intracellular moieties further comprise:
(iii) a first intracellular domain or a portion thereof.

3. The modified immune cell of claim 1 or 2, wherein the one or more intracellular moieties comprise, in order (from cellular membrane proximal to distal):
(i) the first JAK binding domain or portion thereof, the first STAT binding domain or portion thereof, and the first intracellular domain or portion thereof;
(ii) the first STAT binding domain or portion thereof, the first JAK binding domain or portion thereof, and the first intracellular domain or portion thereof;
(iii) the first JAK binding domain or portion thereof, the first intracellular domain or portion thereof, and the first STAT binding domain or portion thereof;
(iv) the first STAT binding domain or portion thereof, the first intracellular domain or portion thereof, and the first JAK binding domain or portion thereof;
(v) the first intracellular domain or portion thereof, the first JAK binding domain or portion thereof, and the first STAT binding domain or portion thereof;
(vi) the first intracellular domain or portion thereof, the first STAT binding domain or portion thereof, and the first JAK binding domain or portion thereof;
(vii) the first JAK binding domain or portion thereof and the first STAT binding domain or portion thereof, or
(viii) the first STAT binding domain or portion thereof and the first JAK binding domain or portion thereof.

4. The modified immune cell of any one of claims 1-3, wherein the one or more intracellular moieties further comprise:
(iv) a second JAK binding domain or a portion thereof,
(v) a second STAT binding domain or a portion thereof,
(vi) a second intracellular domain or a portion thereof,
or combinations thereof.

5. The modified immune cell of any one of claims 1-4, wherein the first JAK binding domain or portion thereof is derived from a first cytokine receptor.

6. The modified immune cell of any one of claims 1-5, wherein the first STAT binding domain or portion thereof is derived from a second cytokine receptor.

7. The modified immune cell of any one or claims 1-6, wherein the second JAK binding domain or portion thereof is derived from a third cytokine receptor.

8. The modified immune cell of any one or claims 1-7, wherein the second STAT binding domain or portion thereof is derived from a fourth cytokine receptor.

9. The modified immune cell of any one of claims 5-8, wherein the first cytokine receptor and the second cytokine receptor are different cytokine receptors.

10. The modified immune cell of claim 5 or 6, wherein the first cytokine receptor and the second cytokine receptor are the same cytokine receptor.

11. The modified immune cell of any one of claims 8-10, wherein the third cytokine receptor and the fourth cytokine receptor are different cytokine receptors.

12. The modified immune cell of any one of claims 8-10, wherein the third cytokine receptor and the fourth cytokine receptor are the same cytokine receptor.

13. The modified immune cell of any one of claims 7-12, wherein the first cytokine receptor and the third cytokine receptor are different cytokine receptors.

14. The modified immune cell of any one of claims 7-12, wherein the first cytokine receptor and the third cytokine receptor are the same cytokine receptor.

15. The modified immune cell of any one of claims 8-14, wherein the second cytokine receptor and the fourth cytokine receptor are different cytokine receptors.

16. The modified immune cell of any one of claims 8-14, wherein the second cytokine receptor and the fourth cytokine receptor are the same cytokine receptor.

17. The modified immune cell of any one of claims 5-16, wherein the first cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

18. The modified immune cell of any one of claims 6-17, wherein the second cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL 7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR¹, TNFR2, TREM1, TREM2, or Tyro3.

19. The modified immune cell of any one of claims 7-18, wherein the third cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

20. The modified immune cell of any one of claims 8-19, wherein the fourth cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL 7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

21. The modified immune cell of any one of claims 1-20, wherein the first JAK binding domain or portion thereof binds JAK1, JAK2, JAK3, TYK2, or combinations thereof.

22. The modified immune cell of any one of claims 4-21, wherein the second JAK binding domain or portion thereof binds JAK1, JAK2, JAK3, TYK2, or combinations thereof.

23. The modified immune cell of any one of claims 1-22, wherein the first STAT binding domain or portion thereof binds STAT1, STAT2, STAT3, STAT4, STATSA, STAT5B, STAT6, a minimal STAT1 binding motif, a minimal STAT3 binding motif, a minimal STAT5A binding motif, a minimal STAT6 binding motif, or combinations thereof.

24. The modified immune cell of any one of claims 4-23, wherein the second STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof.

25. The modified immune cell of any one of claims 2-24, wherein the first intracellular domain or portion thereof is or comprises a CD35, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPα, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL12R, IL13R, IL 14R, IL15R, IL 17R, IFNaR, IFNgR, TNFR, CSFIR, CSF2R, Dap10, CD36, Dectin-1, ICOSL, or Syk intracellular domain, a portion of any of the foregoing, or combinations thereof.

26. The modified immune cell of any one of claims 4-25, wherein the second intracellular domain or portion thereof is or comprises a CD34, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPα, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL12R, IL13R, IL 14R, IL15R, IL 17R, IFNaR, IFNgR, TNFR, CSFIR, CSF2R, Dap10, CD36, Dectin-1, ICOSL, or Syk intracellular domain, a portion of any of the foregoing, or combinations thereof.

27. The modified immune cell of any one of claims 2-26, wherein the first intracellular domain comprise a CD34 intracellular domain or an FcRγ intracellular domain.

28. The modified immune cell of any one of claims 1-27, wherein the extracellular domain is or comprises an scFv, VHH antibody, centyrin, darpin, or nanobody.

29. The modified immune cell of any one of claims 1-28, wherein the transmembrane domain is or comprises a CD8a, CD28, CD40, MyD88 CD64, CD32a, CD32c, CD16a, CD3zeta, ICOS, Dectin-1, DNGR1, SLAMF7, TRL1, TLR2, TLR3, TRL4, TLR5, TLR6, TLR7, TLR8, or TLR9 transmembrane domain.

30. The modified immune cell of any one of claims 1-29, wherein the CAR further comprises an extracellular leader domain.

31. The modified immune cell of claim 30, wherein the extracellular leader domain comprises a CD8a extracellular leader domain.

32. The modified immune cell of any one of claims 1-31, wherein the CAR further comprises an extracellular hinge domain.

33. The modified immune cell of claim 32, wherein the extracellular hinge domain comprises: a CD8a extracellular hinge domain, a CD28 extracellular hinge domain, a DNGR-1 extracellular hinge domain, a Dectin-1 extracellular hinge domain, or an IgG4 extracellular hinge domain.

34. The modified immune cell of any one of claims 1-33, wherein the CAR has or comprises:
(a) an amino acid sequence selected from Table 2;
(b) an amino acid sequence that differs from a sequence selected from Table 2 by no more than five substitutions, additions, or deletions; or
(c) an amino acid sequence that is at least 80% identical to a sequence selected from Table 2.

35. A pharmaceutical composition comprising a modified immune cell of any one of the previous claims.

36. The pharmaceutical composition of claim 35, wherein the pharmaceutical composition comprises a pharmaceutically acceptable carrier.

37. A chimeric antigen receptor (CAR) comprising:
(a) an extracellular domain;
(b) a transmembrane domain; and
(c) one or more intracellular moieties comprising:
(i) a first JAK binding domain or a portion thereof, and
(ii) a first STAT binding domain or a portion thereof.

38. The CAR of claim 37, wherein the one or more intracellular moieties further comprise:
(iii) a first intracellular domain or a portion thereof.

39. The CAR of claim 37 or 38, wherein the one or more intracellular moieties are in order (from cellular membrane proximal to distal):
(i) the first JAK binding domain or portion thereof, the first STAT binding domain or portion thereof, and the first intracellular domain or portion thereof;
(ii) the first STAT binding domain or portion thereof, the first JAK binding domain or portion thereof, and the first intracellular domain or portion thereof;
(iii) the first JAK binding domain or portion thereof, the first intracellular domain or portion thereof, and the first STAT binding domain or portion thereof;
(iv) the first STAT binding domain or portion thereof, the first intracellular domain or portion thereof, and the first JAK binding domain or portion thereof;
(v) the first intracellular domain or portion thereof, the first JAK binding domain or portion thereof, and the first STAT binding domain or portion thereof;
(vi) the first intracellular domain or portion thereof, the first STAT binding domain or portion thereof, and the first JAK binding domain or portion thereof;
(vii) the first JAK binding domain or portion thereof and the first STAT binding domain or portion thereof; or
(viii) the first STAT binding domain or portion thereof and the first JAK binding domain or portion thereof.

40. The CAR of any one of claims 37-39, wherein the one or more intracellular moieties further comprise:
(iv) a second JAK binding domain or a portion thereof,
(v) a second STAT binding domain or a portion thereof,
(vi) a second intraceullular domain or a portion thereof,
or combinations thereof.

41. The CAR of any one of claims 37-40, wherein the first JAK binding domain or portion thereof is derived from a first cytokine receptor.

42. The CAR of any one of claims 37-41, wherein the first STAT binding domain or portion thereof is derived from a second cytokine receptor.

43. The CAR of any one or claims 40-42, wherein the second JAK binding domain or portion thereof is derived from a third cytokine receptor.

44. The CAR of any one or claims 40-43, wherein the second STAT binding domain or portion thereof is derived from a fourth cytokine receptor.

45. The CAR of any one of claims 42-44, wherein the first cytokine receptor and the second cytokine receptor are different cytokine receptors.

46. The CAR of any one of claims 42-44, wherein the first cytokine receptor and the second cytokine receptor are the same cytokine receptor.

47. The CAR of any one of claims 44-46, wherein the third cytokine receptor and the fourth cytokine receptor are different cytokine receptors.

48. The CAR of any one of claims 44-46, wherein the third cytokine receptor and the fourth cytokine receptor are the same cytokine receptor.

49. The CAR of any one of claims 43-48, wherein the first cytokine receptor and the third cytokine receptor are different cytokine receptors.

50. The CAR of any one of claims 43-48, wherein the first cytokine receptor and the third cytokine receptor are the same cytokine receptor.

51. The CAR of any one of claims 44-50, wherein the second cytokine receptor and the fourth cytokine receptor are different cytokine receptors.

52. The CAR of any one of claims 44-50, wherein the second cytokine receptor and the fourth cytokine receptor are the same cytokine receptor.

53. The CAR of any one of claims 41-52, wherein the first cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

54. The CAR of any one of claims 42-53, wherein the second cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

55. The CAR of any one of claims 43-54, wherein the third cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

56. The CAR of any one of claims 44-55, wherein the fourth cytokine receptor is or comprises: 4-1BB, Axl, CD30, CD40, CNTFR, CSF2Rb, EPOR, FLT3, GCSFR, GHR, GMCSFRa, gp130, IFNAR2, IFNGR1, IL2Rb, IL3Ra, IL4Ra, IL5Ra, IL6Ra, IL7Ra, IL9Ra, IL10Ra, IL11Ra, IL20Ra, IL21Ra, IL22R, IL27Ra, IL28R, IL31Ra, LIFRb, MerTK, minimal STAT1 binding motif pYxxP, minimal STAT3 binding motif pYxxQ, minimal STAT5a binding motif pYxxL, minimal STAT6 binding motif pYxxF, MyD88, OX40, PRLR, TNFR1, TNFR2, TREM1, TREM2, or Tyro3.

57. The CAR of any one of claims 37-56, wherein the first JAK binding domain or portion thereof binds JAK1, JAK2, JAK3, TYK2, or combinations thereof.

58. The CAR of any one of claims 40-57, wherein the second JAK binding domain or portion thereof binds JAK1, JAK2, JAK3, TYK2, or combinations thereof.

59. The CAR of any one of claims 37-58, wherein the STAT binding domain or portion thereof binds STAT1, STAT2, STAT3, STAT4, STATSA, STATSB, STAT6, a minimal STAT1 binding motif, a minimal STAT3 binding motif, a minimal STAT5a binding motif, a minimal STAT6 binding motif, or combinations thereof.

60. The CAR of any one of claims 40-59, wherein the second STAT binding domain or portion thereof binds STAT1, STAT2, STAT3, STAT4, STAT5A, STAT5B, STAT6, a minimal STAT1 binding motif, a minimal STAT3 binding motif, a minimal STAT5A binding motif, a minimal STAT6 binding motif, or combinations thereof.

61. The CAR of any one of claims 38-60, wherein the first intracellular domain or a portion thereof is or comprises a CD34, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, ILIR, IL2R, IL3R, IL4R, IL5R, IL6R, IL7R, IL8R, IL9R, IL10R, IL11R, IL12R, IL13R, IL14R, IL15R, IL17R, IFNaR, IFNgR, TNFR, CSFIR, CSF2R, Dap10, CD36, Dectin-1, ICOSL, or Syk intracellular domain, a portion of any of the foregoing, or combinations thereof.

62. The CAR of any one of claims 40-61, wherein the second intracellular domain or portion thereof is or comprises a CD34, FcRγ, MyD88, CD40, CD64, CD32a, CD32c, CD16a, CD89, TLR1, TLR2, TLR3, TLR4, TLR5, TLR6, TLR7, TLR8, TLR9, ALK, AXL, DDR2, EGFR, EphA1, INSR, cMET, MUSK, PDGFR, PTK7, RET, ROR1, ROS1, RYK, TIE2, TRK, VEGFR, CD19, CD20, 41BB, CD28, GCSFR (CD114), RAGE, CD30, CD160, DR3, Fn14, HVEM, CD160, NGFR, RANK, TNFR2, TROY, XEDAR, TRIF, OX40, GITR, TREM-1, TREM-2, DAP12, MR, ICOS, MyD88, V/I/LxYxxL/V, SIRPa, CD45, Siglec-10, PD1, SHP-1, SHP-2, KIR-2DL, KIR-3DL, NKG2A, CD170, CD33, BTLA, CD32b, SIRPb, CD22, PIR-B, LILRB1, 41BBL (TNFSF9), CD27, OX40L, CD32b, CD11b, ITGAM, SLAMF7, CD206, CD163, CD209, Dectin-2, IL1R, IL2R, IL3R, IL4R, IL5R, IL6R, IL7R, IL8R, IL9R, IL10R, IL11R, IL12R, IL 13R, IL14R, IL 15R, IL17R, IFNaR, IFNgR, TNFR, CSFIR, CSF2R, Dap10, CD36, Dectin-1, ICOSL, or Syk intracellular domain, a portion of any of the foregoing, or combinations thereof.

63. The CAR of any one of claims 38-62, wherein the first intracellular domain comprises a CD3ζ intracellular domain or an FcRγ intracellular domain.

64. The CAR of any one of claims 37-63, wherein the extracellular domain is or comprises an scFv, VHH antibody, centyrin, darpin, or nanobody.

65. The CAR of any one of claims 37-64, wherein the transmembrane domain is or comprises a CD8a, CD28, CD40, MyD88 CD64, CD32a, CD32c, CD16a, CD3zeta, ICOS, Dectin-1, DNGR1, SLAMF7, TRL1, TLR2, TLR3, TRL4, TLR5, TLR6, TLR7, TLR8, or TLR9 transmembrane domain.

66. The CAR of any one of claims 37-65, wherein the CAR further comprises an extracellular leader domain.

67. The CAR of claim 66, wherein the extracellular leader domain comprises a CD8a extracellular leader domain.

68. The CAR of any one of claims 37-67, wherein the CAR further comprises an extracellular hinge domain.

69. The CAR of claim 68, wherein the extracellular hinge domain comprises: a CD8a extracellular hinge domain, a CD28 extracellular hinge domain, a DNGR-1 extracellular hinge domain, a Dectin-1 extracellular hinge domain, or an IgG4 extracellular hinge domain.

70. The CAR of any one of claims 37-69, wherein the CAR has or comprises:
(a) an amino acid sequence selected from Table 2;
(b) an amino acid sequence that differs from a sequence selected from Table 2 by no more than five substitutions, additions, or deletions; or
(c) an amino acid sequence that is at least 80% identical to a sequence selected from Table 2.

71. A nucleic acid construct comprising one or more nucleic acid sequences encoding:
(a) an extracellular domain;
(b) a transmembrane domain; and
(c) one or more intracellular moieties comprising:
(i) a first JAK binding domain or a portion thereof, and
(ii) a first STAT binding domain or a portion thereof;
wherein the nucleic acid construct encodes a chimeric antigen receptor (CAR) comprising (a) through (c).

72. The nucleic acid construct of claim 71, wherein the one or more intracellular moieties further comprise:
(iii) a first intracellular domain or a portion thereof.

73. The nucleic acid construct of claim 71 or 72, wherein the one or more intracellular moieties further comprise:
(iv) a second JAK binding domain or a portion thereof,
(v) a second STAT binding domain or a portion thereof,
(vi) a second intraceullular domain or a portion thereof,
or combinations thereof.

74. The nucleic acid construct of claim 71 or 72, further comprising one or more nucleic acid sequences encoding:
(d) one or more extracellular leader domains,
(e) one or more extracellular hinge domains,
(f) one or more cleavage peptides,
or combinations thereof.

75. The nucleic acid construct of claim 74, wherein the cleavage peptide is or comprises a P2A, F2A, E2A or T2A peptide.

76. The nucleic acid construct of any one of claims 71-75, wherein the nucleic acid construct has or comprises:
(a) a nucleotide sequence selected from Table 3;
(b) a nucleotide sequence that differs from a sequence selected from Table 3 by no more than five substitutions, additions, or deletions; or
(c) a nucleotide acid sequence that is at least 80% identical to a sequence selected from Table 3.

77. The nucleic acid construct of any one of claims 71-76, wherein the nucleic acid construct comprises DNA or messenger RNA (mRNA).

78. The nucleic acid construct of any one of claims 71-77, wherein the nucleic acid construct comprises a modification selected from: a modified nucleotide, an alteration to the 5′ untranslated region (UTR), an alteration to the 3′ UTR, a cap structure, a poly(A) tail, or combinations thereof.

79. The nucleic acid construct of claim 78, wherein the cap structure comprises AGCap1, m6AGCap1, or Anti-Reverse Cap Analog (ARCA).

80. The nucleic acid construct of claim 78 or 79, wherein the modified nucleotide comprises pseudouridine (PsU), 5-methoxyuridine (5moU), 5-methylcytidine/pseudouridine (5meC PsU), N1-methyl-pseudouridine (N1mPsU), or combinations thereof.

81. The nucleic acid construct of any one of claims 71-80, wherein the nucleic acid construct is a purified nucleic acid construct.

82. The nucleic acid construct of any one of claims 71-81, wherein the nucleic acid construct is codon-optimized.

83. The nucleic acid construct of claim 82, wherein the nucleic acid construct is codon-optimized for expression in a stem cell, monocyte, macrophage, or dendritic cell.

84. A pharmaceutical composition comprising the nucleic acid construct of any one of claims 71-83.

85. The pharmaceutical composition of claim 84, wherein the pharmaceutical composition comprises a pharmaceutically acceptable carrier.

86. A method of treating a disease or disorder in a subject, the method comprising:
administering to the subject a therapeutically effective amount of the pharmaceutical composition of any one of claim 35, 36, 84, or 85,
wherein at least one sign or symptom of the disease or disorder is improved in the subject after administration.

87. The method of claim 86, wherein the step of administering is or comprises transarterial, subcutaneous, intravenous, intradermal, intratumoral, intranodal, intramedullary, intramuscular, or intraperitoneal delivery.

88. A method of modifying an immune cell, the method comprising:
delivering to the immune cell a nucleic acid construct of any one of claims 71-83, thereby producing a modified immune cell,
wherein the modified immune cell is or comprises a macrophage, monocyte, dendritic cell, or stem cell.

89. The method of claim 88, wherein the nucleic acid construct comprises DNA or messenger RNA (mRNA).

90. The method of claim 88 or 89, wherein the nucleic acid construct comprises a modification selected from: a modified nucleotide, an alteration to the 5′ untranslated region (UTR), an alteration to the 3′ UTR, a cap structure, a poly(A) tail, or combinations thereof.

91. The method of claim 90, wherein the cap structure comprises AGCap1, m6AGCap1, or Anti-Reverse Cap Analog (ARCA).

92. The method of claim 90 or 91, wherein the modified nucleotide comprises pseudouridine (PsU), 5-methoxyuridine (5moU), 5-methylcytidine/pseudouridine (5meC PsU), N1-methyl-pseudouridine (NImPsU), or combinations thereof.

93. The method of any one of claims 88-92, wherein the nucleic acid construct is a purified nucleic acid construct.

94. The method of claim 93, wherein the purified nucleic acid construct is produced by a method comprising silica membrane purification, high performance liquid chromatography (HPLC), Dynabeads, LiCl precipitation, phenol-chloroform extraction, resin based purification, poly A isolation, RNeasy, or combinations thereof.

95. The method of any one of claims 88-94, wherein the nucleic acid construct is codon-optimized.

96. The method of claim 95, wherein the nucleic acid construct is codon-optimized for expression in a stem cell, monocyte, macrophage, or dendritic cell.

97. The method of any one of claims 88-96, wherein the delivering comprises electroporation or transfection with the nucleic acid construct.

98. The method of any one of claims 88-96, wherein the nucleic acid construct is encapsulated within a delivery vehicle.

99. The method of claim 98, wherein the delivery vehicle is or comprises a liposome, a lipid nanoparticle, a polymer, an adeno-associated viral (AAV) vector, an adenoviral vector, a retroviral vector or combinations thereof.

100. The method of claim 99, wherein the liposome or lipid nanoparticle comprises one or more cationic lipids, one or more non-cationic lipids, one or more cholesterol-based lipids, one or more PEG-modified lipids, or combinations thereof.

101. The method of claim 99, wherein the retroviral vector comprises a lentiviral vector or a gammaretroviral vector.

102. The method of claim 101, wherein the lentiviral vector is packaged with a Vpx protein.

103. The method of claim 99, wherein the adenoviral vector comprises an Ad2 vector or an Ad5 vector.

104. The method of claim 103, wherein the Ad5 vector comprises an Ad5f35 adenoviral vector.

105. The method of any one of claims 88-104, the method further comprising delivering to the immune cell an additional payload.

106. The method of claim 105, wherein the additional payload is or comprises a pathogen recognition receptor agonist, polyinosinic: polycytidylic acid (poly I:C), a TLR7/8 agonist, a CpG oligodeoxynucleotide, a NOD-like receptor (NLR) agonist, a RIG-I-like receptor (RLR) agonist, a C-type lectins receptor (CLR) agonist, a cytosolic DNA sensing, the cyclic GMP-AMP synthase-stimulator of interferon genes (cGAS-STING) agonist, an interferon-inducible protein 16 (IFI16) agonist, a DEAD-box helicase 41 (DDX41) agonist, an LRR binding FLII interacting protein 1 (LRRFIP1) agonist, an absent in melanoma 2 (AIM2) agonist, an aryl hydrocarbon receptor (AhR) ligand, or combinations thereof.

107. The method of claim 105 or 106, wherein the nucleic acid construct and the additional payload are encapsulated within the delivery vehicle.


SEQUENCE LISTING (166 sequences)

SEQ ID NO: 1 - protein, 448 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 445 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 375 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRLKIQVRKAAITSYEKSDGVYTGLSTRNQETYETLKHEKPPQ

SEQ ID NO: 4 - protein, 545 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSG

SEQ ID NO: 5 - protein, 475 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSG

SEQ ID NO: 6 - protein, 545 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 475 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGRLKIQVRKAAITSYEKSDGVYTGLSTRNQETYETLKHEKPPQ

SEQ ID NO: 8 - protein, 542 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSG

SEQ ID NO: 9 - protein, 542 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSPNRKNPLWPSVPDPAHSSLGSWVPTIMEEDAFQLPGLGTPPITKLTVLEEDEKKPVPWESHNSSETCGLPTLVQTPTSFGFDKPHVLVDLLVDDSG

SEQ ID NO: 10 - protein, 472 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRLKIQVRKAAITSYEKSDGVYTGLSTRNQETYETLKHEKPPQSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSG

SEQ ID NO: 11 - protein, 472 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRLKIQVRKAAITSYEKSDGVYTGLSTRNQETYETLKHEKPPQSPNRKNPLWPSVPDPAHSSLGSWVPTIMEEDAFQLPGLGTPPITKLTVLEEDEKKPVPWESHNSSETCGLPTLVQTPTSFGFDKPHVLVDLLVDDSG

SEQ ID NO: 12 - protein, 542 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 472 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGRLKIQVRKAAITSYEKSDGVYTGLSTRNQETYETLKHEKPPQ

SEQ ID NO: 14 - DNA, 1347 bp, synthetic construct
  source 1..1347: mol_type = other DNA
atggccttaccagtgaccgccttgctcctgccgctggccttgctgctccacgccgccaggccggacattcaaatgacacagagtccctcatccctcagtgccagcgtgggcgatcgggtgactataacctgcagagcttctcaggacgtgaataccgctgtggcgtggtaccagcagaagccaggcaaagcgcctaagcttctcatttatagtgccagcttcctgtactcaggtgttccgtctcgcttttctggaagtagaagtgggaccgatttcacattgacgatcagcagcttgcagcccgaagatttcgccacctactactgtcagcagcactacactaccccaccgacatttggtcaaggcacaaaagtagagattaaacgcactggttccaccagcgggagcgggaaacccggctctggggaggggagcgaggtccagctggtggaatccgggggtggtcttgtgcagccaggaggatccttgaggttgtcctgcgccgcaagcggctttaacatcaaagatacatacatccattgggtccgacaggcccctggaaagggcctggagtgggtcgcccggatctacccaactaacgggtacactcgctacgctgatagcgtcaagggtcggtttactatttctgccgacacctcaaaaaacacagcctacctccagatgaactctctcagagctgaggatacagccgtgtactattgcagccggtggggaggcgacgggttctacgctatggatgtgtgggggcagggcacactggtcaccgtgagctcatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 15 - DNA, 1338 bp, synthetic construct
  source 1..1338: mol_type = other DNA
atggccttaccagtgaccgccttgctcctgccgctggccttgctgctccacgccgccaggccggacattcaaatgacacagagtccctcatccctcagtgccagcgtgggcgatcgggtgactataacctgcagagcttctcaggacgtgaataccgctgtggcgtggtaccagcagaagccaggcaaagcgcctaagcttctcatttatagtgccagcttcctgtactcaggtgttccgtctcgcttttctggaagtagaagtgggaccgatttcacattgacgatcagcagcttgcagcccgaagatttcgccacctactactgtcagcagcactacactaccccaccgacatttggtcaaggcacaaaagtagagattaaacgcactggttccaccagcgggagcgggaaacccggctctggggaggggagcgaggtccagctggtggaatccgggggtggtcttgtgcagccaggaggatccttgaggttgtcctgcgccgcaagcggctttaacatcaaagatacatacatccattgggtccgacaggcccctggaaagggcctggagtgggtcgcccggatctacccaactaacgggtacactcgctacgctgatagcgtcaagggtcggtttactatttctgccgacacctcaaaaaacacagcctacctccagatgaactctctcagagctgaggatacagccgtgtactattgcagccggtggggaggcgacgggttctacgctatggatgtgtgggggcagggcacactggtcaccgtgagctcatccggaattgaagtgatgtatccgccgccgtatctggataacgaaaaaagcaacggcaccattattcatgtgaaaggcaaacatctgtgcccgagcccgctgtttccgggcccgagcaaaccgttttgggtgctggtggtggtgggcggcgtgctggcgtgctatagcctgctggtgaccgtggcgtttattattttttgggtgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - DNA, 1128 bp, synthetic construct
  source 1..1128: mol_type = other DNA
atggcacttcccgtcacagcacttctgctcccgctggccctgctcctgcatgccgcacgccctgacatccagatgacccagagcccatcctctctgagcgcgtccgtgggcgacagagtgaccatcacctgtagagcctcccaggacgtcaataccgctgtggcctggtaccaacagaagccaggaaaggccccgaagctgctcatctacagcgcctcattcctgtactccggcgtgcctagcagattctctggtagtcggtccggcacagatttcaccttgacaatcagctctttgcaacctgaggactttgctacctattactgccaacagcactacactacaccgcccacgtttggtcaaggcaccaaagtggagatcaagcggactggtagcacaagtggttcaggtaagcctggaagcggagaaggttctgaggtgcaactcgttgaatctggcggtggcctggtccagcctggcggttccctgcgcctttcttgtgctgcgtccggtttcaacatcaaggacacctacatccattgggttcgtcaggcacccggtaagggactcgagtgggtagcccgcatttatcccacaaacggatacactagatacgctgattccgtcaagggccgttttaccatcagtgccgacacatccaagaatactgcctaccttcagatgaacagcctccgcgccgaagacaccgcggtgtattactgttcccgctggggaggcgatggattctacgcaatggacgtctggggccaggggaccctcgtgacagtctcctcaagcggcatcgaggtcatgtacccgccaccctacctggacaacgagaagtccaacgggacaattatccatgttaagggcaagcacctgtgccctagccccctgttccccggtccctccaaaccgttctgggtcctggtagtggttggcggtgtgttggcttgctactctctcctggttaccgtcgccttcatcattttctgggtacgtctgaagattcaggtgcgtaaagctgcaatcacctcatatgagaaatccgacggggtgtacaccggcctgtccacaaggaaccaggagacctacgagaccctcaagcacgagaagcctccacagtaa

SEQ ID NO: 17 - DNA, 1638 bp, synthetic construct
  source 1..1638: mol_type = other DNA
atggccctgcctgtaacagccctcctgctcccactggctctgctcctgcatgcggccaggcctgacatccaaatgacacaaagcccctctagcttgtccgcaagtgtcggtgatagggtgacgattacttgccgtgcctcccaggacgtcaacaccgctgttgcttggtaccaacagaagccgggcaaagcgccgaaactcctgatctactctgcaagtttcctgtacagcggtgtccctagtcggttctctggcagccgctcaggaactgatttcacactgaccatcagttccctgcagcctgaggatttcgctacctattactgccaacagcattacactaccccacccaccttcggacaaggaaccaaagttgaaatcaagcgcaccggcagtacgtctggctctgggaagcccggaagcggggagggcagcgaggtccagttggtcgagtctgggggcgggctggtgcagcctgggggatccctgagattgtcatgcgccgcaagcggctttaacatcaaagacacttacatccattgggtgcggcaagctcccgggaagggcctggagtgggtagcacgcatctatcccaccaatggctacacccgctacgccgattccgtgaagggtcgctttaccatctctgcagacacaagtaagaataccgcctatctgcagatgaactcactgagagcagaggatacagctgtttattactgctcccggtggggtggcgatggtttttatgctatggacgtatggggccagggcaccctggttaccgtctctagctccggtaccacaacccctgctccacgcccgcccactccggcccctactattgccagccaacccttgagtctcaggcctgaagcctgtcgccccgcggccggaggcgcagtgcatacacgcggcctggatttcgcctgtgacatttatatctgggcacccttggccggcacctgcggtgttctgcttctgagcttggtgatcactttgtattgccgtgtgaagttcagccgctctgctgatgcacctgcctatcaacaggggcagaaccagctctataacgagttgaacctgggccggcgcgaggaatacgatgtcctcgataaacgcagaggacgtgatccagagatgggtggaaagccacgccgtaagaacccccaggagggtctgtacaatgagctgcagaaggataaaatggcagaggcttacagcgaaattgggatgaagggggagcggcgccgtggcaaggggcatgacggcctttatcaaggcctgtccaccgccaccaaggatacttatgacgccctgcacatgcaagcgctgcccccgcggtcccccaatcgcaagaacccgctgtggcctagcgtaccagatcccgcgcattccagcctgggctcttgggtgccgacaatcatggaagaggacgctttccagctgcctggtctgggcactcctcccattacgaaactgaccgtgctcgaggaagatgagaaaaagcccgtgccttgggagtctcataactccagcgagacatgcggtctgcccaccctcgtgcagacccccacgagctttgggtacgataaaccccacgtgttggtcgacttgctcgtcgatgacagcggctag

SEQ ID NO: 18 - DNA, 1428 bp, synthetic construct
  source 1..1428: mol_type = other DNA
atggctctcccggtgacggctttgctgctccccctggccctcctgttgcacgctgccagacctgacatccagatgacccagtccccgtccagcctgagtgcgtccgtgggcgaccgcgtgacaatcacctgccgcgcatcccaggatgttaatactgctgtcgcgtggtaccaacagaagcccggcaaggcaccaaagctcctgatttactctgcctcttttctctacagcggagtaccctcccgctttagcggatcccgctctggcacggacttcaccctgaccattagctccctgcagcctgaggatttcgcaacctattactgtcagcaacactacaccactcctcccaccttcggtcagggcactaaggtcgaaatcaagcgcaccggtagcaccagtggctcaggtaaacctggcagcggggagggctctgaggtgcagctcgtcgagtccggagggggcttggtgcagcccggtggctccctgcgcctgtcctgcgccgcgtctggatttaacatcaaagacacttacatccattgggtcagacaggctcctggcaaaggcctggagtgggtagcgcgtatctaccctaccaacggctacacccgctatgcggactccgtgaagggacgcttcactatttccgctgataccagcaagaacaccgcatatttgcaaatgaacagccttagagcggaagacactgcagtctactattgctctcggtggggaggcgatgggttctacgccatggacgtgtggggtcagggcaccctggtcactgtgtcttccagcggtaccacgacccctgctccccgtccacccaccccagcccctaccattgcatcacagcccctgtccctgcggcccgaggcatgtcgccccgcggccgggggagctgtccatactcgtgggctggactttgcctgtgatatctatatctgggctcccctcgcaggtacctgtggggtcctgctcctgagcctggtgatcaccctgtactgccgcctgaagattcaggtgcgtaaagcggctattacttcctacgagaagagcgacggtgtgtatactggcctgtcaacccgtaaccaagaaacctacgagaccctcaagcatgagaaaccacctcagagtccaaatcggaagaacccactctggccgtccgttcctgaccccgcccattcctctctcggctcatgggtgcctactatcatggaggaagacgctttccagctgccgggcctcggcacgccgccaatcaccaagctgaccgtgctggaggaagatgaaaaaaagcctgttccctgggaatcccacaactccagtgagacctgcggcctccctactttggtgcagaccccaacatcctttgggtacgataaaccgcacgtcctcgtggacctgctcgtcgacgattccggctaa

SEQ ID NO: 19 - DNA, 1638 bp, synthetic construct
  source 1..1638: mol_type = other DNA
atggctctgcctgtgacagctctcctgctcccactcgccctgttgctgcatgcagcccgccccgatatccagatgacccaatccccgagctccctgtcagccagcgtgggcgaccgcgtgaccatcacatgccgcgccagccaggacgtgaacacggcggtggcatggtaccaacagaaacccgggaaggcccctaagctgcttatttacagtgcgtctttcctgtattcaggcgtgccatcccgcttctcagggtccaggtcaggcaccgacttcacccttaccatctccagcctgcagcccgaagacttcgctacctactattgccaacagcattacaccactccccctaccttcggccaggggaccaaggtcgagattaagcgtacaggctctacgagcgggagcggcaagccaggttccggtgaggggtctgaggtgcagcttgtcgaatccgggggcgggctggtgcaaccagggggttccctgcggctcagttgcgctgcctccgggttcaatatcaaggacacgtatatccattgggtgagacaggctcccggcaaaggcctcgagtgggtggctaggatctacccaaccaacggatataccagatacgctgacagcgtgaagggccgcttcaccatttctgcagatacctctaagaacaccgcgtacctgcagatgaacagcctgcgggcggaggacactgccgtgtactattgtagcagatggggaggcgacggcttttacgccatggatgtctggggccaggggaccctggtgacagttagttcctctggcacaacgactccagcacccagaccccctacacctgcccccacaattgctagccagcccctgtctctgcgccccgaggcctgccgtcccgccgcgggcggggccgtccatacgcggggcctcgacttcgcttgcgatatctatatctgggctcctctggccggcacgtgtggggtgctgctcctgtccctggtcatcactctgtattgttctcctaataggaagaaccccctgtggccctccgtcccggacccagcccacagctctctggggtcctgggtgccgaccatcatggaggaagacgcattccagctccccggtctgggtaccccgcccatcaccaagctgaccgtgctggaggaagacgaaaaaaagcccgtcccctgggagtcccacaactcaagtgagacttgtggactgcctacacttgttcagacccccacatccttcggctatgacaagcctcatgtcctggttgacctcctggtcgacgatagcggccgcgtgaagttctccaggtctgctgatgccccggcgtaccaacagggtcagaaccagctgtacaacgagcttaatttggggcggcgcgaagagtatgacgtccttgacaagagacgcggccgcgaccccgagatgggcggtaaaccccgcaggaagaacccccaggagggactctacaatgaactgcagaaggacaaaatggctgaggcatatagcgaaatcggcatgaagggcgagagacggcgtggcaagggtcacgacggcctgtatcaaggcctgagcaccgcgaccaaggatacatacgatgcgttgcacatgcaagcattgccccctcgctag

SEQ ID NO: 20 - DNA, 1428 bp, synthetic construct
  source 1..1428: mol_type = other DNA
atggcgctgcctgtcacagcgctcttgctgccacttgctctgttgctccacgctgcaagacccgacatccagatgactcagtccccgagctctttgtccgcatccgtgggggaccgcgtgactatcacctgtcgggcgagtcaagacgtgaataccgccgtcgcatggtaccaacagaagcccggaaaggccccaaagcttttgatctattctgcttcttttctgtattccggcgtcccctctcgcttctccgggagccgttccggcaccgacttcaccctgaccatcagctctctgcagcccgaagacttcgctacatattactgtcaacagcattatactaccccacccaccttcggccagggcaccaaagtcgaaatcaagagaaccggctccacttctggctccggtaagccaggctctggggaaggcagcgaggtgcagctggtcgaatctggcggtgggttggtacagcccggcggatctctccgcctctcttgtgccgcttccggcttcaacatcaaggacacatacatccactgggtgcggcaggctcctggtaagggcctggaatgggtcgccaggatctatcctacaaacggctacacaaggtacgccgactccgtgaagggccgctttaccatcagtgccgatacttccaagaataccgcctacctgcaaatgaactcactgcgcgctgaggatactgctgtctactattgcagccggtggggcggagacggtttctacgccatggatgtctggggacaggggaccctggtgaccgtaagctcttccggtacaaccacacctgcgcctaggccccctacgccggcccctactattgccagccaaccactttctctgcgcccggaggcgtgccgccctgcagccggcggtgccgtccacacccgcggcctggattttgcctgcgacatctatatttgggctcctctggcgggaacctgtggcgtgctgctcttgtctttggtgatcaccctgtactgttcccctaaccgtaagaaccctctgtggcccagcgtgcctgatccggcacattcttccttgggctcttgggtgccgaccatcatggaggaagatgcttttcagctgcctggactgggtacgccgcctatcaccaagctgacagtcttggaagaggatgagaaaaagcctgtaccatgggagtctcataatagctctgagacctgtggcttgcctactctggtgcagacacctacttccttcggctatgacaagccccatgtcctggtggatcttctggtggatgacagcggcagactgaagatccaggtgcgtaaggcagccatcacttcctatgaaaagtccgacggcgtgtataccggtctgtcaactcgcaatcaggaaacctacgagacccttaagcacgagaagcctccccaatag

SEQ ID NO: 21 - DNA, 1629 bp, synthetic construct
  source 1..1629: mol_type = other DNA
atggccctgcccgttaccgccctgttgctgccactggctctcctgcttcatgccgctcgcccagatattcagatgactcagtctcctagctctctgagcgctagcgtgggagaccgcgtcaccatcacatgccgcgccagccaggacgtgaacaccgccgtggcgtggtaccaacagaagcccggaaaagctcccaagctgctcatttactccgcatctttcctgtattccggcgtgcccagtcgttttagcggctcaaggtctggaaccgactttacactgactattagctctctgcaaccagaggactttgcaacttactattgccaacagcactacactacaccacctacatttggccagggcacaaaggttgagatcaagcgcaccgggagtacatccggttccggcaagcccgggtctggcgagggctccgaggtacagctggtcgagagcgggggaggcctggtccagccgggtggcagcctgcgtctcagctgcgccgcatccggctttaacattaaggacacctatatccattgggtgcgtcaggctccaggtaagggacttgagtgggtggcgcggatttaccctacgaacggatacacaaggtacgctgacagcgtgaaaggacgctttaccatcagcgccgatacgagcaagaataccgcttacctccagatgaattccctgcgcgccgaggacaccgcggtgtattactgctctcgctggggtggcgatggcttctatgccatggacgtgtggggacaggggacactggtgaccgtaagctctagtggaatcgaggtgatgtatccgcctccctatctggacaatgagaaaagcaacggcactatcattcatgtgaaaggtaagcatctttgtcccagtcccctgtttcctgggccgtccaagcccttttgggtgctggtggttgtcggcggagtgctggcgtgttattctctgttggtcacagtggcattcattatcttttgggtgcgtgtgaaattttccagatccgctgacgcccccgcataccaacagggtcagaaccagctctacaacgagcttaatctgggccgtagggaagagtacgatgtgcttgataagcgtcgcggtcgtgaccccgagatgggcggtaagcctagacgcaagaacccccaggaggggctgtacaatgagctccagaaggacaagatggccgaagcctactctgagattggcatgaagggcgaaagacgcaggggcaaaggccatgatgggctgtaccagggcctgtctaccgctaccaaagatacatacgatgctctgcatatgcaggccctgcctccccgctcccctaaccgcaaaaacccactctggcctagcgtgcctgatcccgcccacagcagtctcggtagctgggtgcccaccatcatggaagaggacgctttccaattgcctgggctgggcaccccacccattaccaagttgaccgtactcgaagaggacgagaagaaacccgtcccttgggagagtcacaactcttccgagacctgtggcttgcccaccctggtgcagacacctacctccttcggatacgataagccccacgtcctcgtggatctcctggtcgacgatagtggttag

SEQ ID NO: 22 - DNA, 1629 bp, synthetic construct
  source 1..1629: mol_type = other DNA
atggctctgcctgtgaccgccttgctcctgccgctggcactgctcctgcacgctgcacgccccgatatccagatgacgcagtctccaagctctctgtcagcctccgtgggagaccgcgtgacaattacatgccgcgcctctcaggacgttaatacagccgtggcctggtatcaacagaaacccggaaaggcacccaaacttctgatctactctgcgtcctttctctatagtggcgtaccctcacgtttcagcgggagtagaagcggaaccgactttaccctgacaattagtagcctgcagcccgaggatttcgccacgtactattgtcaacagcactacactacccctcccaccttcggccaggggactaaagtagaaattaagcgcaccggttctacttccgggagcggcaaaccggggtccggagagggctctgaggtgcagctcgttgagtccggcggtggattggtccagccgggagggtctctgaggctgtcctgcgcagcttctggcttcaacattaaggacacctacatccattgggtgaggcaggcccctggtaaaggactggagtgggttgccaggatctatcctaccaatggttacacccgttatgcggacagcgtgaagggccggttcaccatctctgcggacacctccaagaacaccgcgtacctgcagatgaactccctccgcgccgaggacacagccgtttactattgtagccgttggggtggcgacgggttttacgctatggacgtgtggggacagggcaccctggttaccgtctctagctctggcatcgaggtgatgtatccccctccatatctcgacaacgagaaatccaacggcaccattatccacgtgaagggtaaacacctgtgcccgtcccctctgttccccggtcccagcaagccattctgggtcctggtagtggtcggtggcgtgctggcgtgttacagccttctggtgaccgtggcgtttattatcttctgggtgcgcgtgaagttcagccgctctgctgacgccccagcctaccaacagggccagaaccagctctacaacgagctcaacctgggcaggcgcgaagagtatgatgtactggacaagaggagaggtcgggacccggagatgggcggaaagccccgcaggaagaaccctcaggaaggcttgtacaacgagctccaaaaagacaaaatggctgaggcctacagcgagatcgggatgaaaggggaacgtcgccgggggaaaggccacgacgggctctatcaggggctgtccacagcaaccaaggatacctacgatgcactccacatgcaggctctgccgcccagaagcccaaaccgcaagaaccccctgtggccctccgtgcccgaccccgcccactcaagcctcggcagctgggtgcctaccatcatggaggaagacgccttccagctccctggtctgggcacgccgcccatcaccaagctgaccgttctggaggaagacgagaaaaagcccgttccctgggagtctcataactccagcgaaacctgtggtctccctaccctcgtgcagacgccgaccagcttcggattcgataagcctcacgtcctggtcgatcttctggtggatgactctggttaa

SEQ ID NO: 23 - DNA, 1419 bp, synthetic construct
  source 1..1419: mol_type = other DNA
atggccttgcctgttacagcgctgttgctgccactggcccttctgcttcacgctgcacgccctgacatccagatgacccagtcccctagttccctgtctgcctctgtgggagaccgggtcactatcacatgcagagcaagtcaagacgtgaataccgccgtggcctggtaccaacagaagccaggcaaggcaccaaagttgctgatctacagcgcctccttcctgtacagcggcgtcccctcccgcttttccggctcccgctcagggaccgatttcacccttaccatcagctcccttcaaccggaggattttgccacatattactgccagcaacactacaccactccgcctactttcggacaagggaccaaggtagaaattaagagaaccggctccactagtggctccggtaaaccaggctcaggtgagggatctgaggtccagctcgtggagagcgggggtggactggttcagccgggtggctctctccgcctcagttgcgctgcctccggctttaacattaaggatacatacattcactgggttcgccaggccccaggaaagggcctcgagtgggtggccagaatctacccaactaatggctacactcgttatgctgactccgtgaagggcaggtttaccatcagcgccgacacctctaaaaacaccgcctacctgcagatgaacagtctccgcgccgaggacacggccgtgtattactgctcacgctggggaggcgacggattttacgctatggacgtttgggggcagggcactctggtgacagtgtcttccagcggcatcgaagtcatgtacccaccgccttacctggataacgagaagtccaacggcacaatcattcacgtcaaaggtaaacacctctgtccaagtccgttgttccccggccctagcaaacccttctgggttcttgtggttgtgggtggcgtcctggcttgttatagccttctggtgacagtagcgtttattatcttctgggtgcgtcttaagatccaagtgcgcaaagctgccatcacctcctacgagaagtctgacggtgtatacaccggtctgtcaactcgcaaccaggaaacctatgagacactgaagcacgaaaaacccccgcagagcccgaaccgcaagaaccccctgtggccttcagtgcccgaccctgcccactcctctctgggttcttgggtccccaccatcatggaagaggatgccttccagctcccaggtctcggtacgcccccaattacaaagctgaccgtgctggaagaggacgagaaaaagccagtcccttgggagagccacaatagctctgagacctgcggactccccactctggtgcaaaccccaacctccttcggttatgataaaccgcatgtgcttgtggacctcctggtggacgattctggatag

SEQ ID NO: 24 - DNA, 1419 bp, synthetic construct
  source 1..1419: mol_type = other DNA
atggctcttcccgtcacggccctcctgttgccacttgctctcttgctgcacgctgcccgcccagacatccagatgacacagagccccagctctctcagcgcttctgtaggggatcgcgtcactatcacctgtcgcgccagtcaggacgtaaacactgcggtcgcttggtaccaacagaagcctggaaaggccccgaagcttctgatttactccgcttccttcctgtattctggcgtgcctagccgtttctccgggagtcgctcaggcaccgactttactctgaccatcagctctcttcagcctgaggacttcgccacctattactgccaacagcattatacgactccccctactttcggtcagggcaccaaagttgagatcaagcgtaccggctctacatccggttccggcaaacccggctccggagaagggtccgaagtgcaactggtagagtctgggggcgggctggtgcagcccggcggatccctgcgcctgagttgtgcagccagcggctttaatatcaaggacacttacatccactgggtgcgccaagcacctggaaagggcctggagtgggtggcccgcatctatccaaccaacggctacacacgttatgctgactccgtgaagggacgcttcacaatcagcgccgacacctccaagaacaccgcctatctgcagatgaactccctgcgcgctgaggacactgccgtttactattgctctcgctggggcggtgacggattctacgccatggatgtttggggccagggaaccctggtgacggtgtcctcaagcggcatcgaagttatgtatccaccgccctaccttgacaacgagaagagcaacggcactattatccacgtgaagggcaaacacctgtgccctagcccccttttccccggtcccagtaagcccttctgggtgttggttgtggtcggtggagtgttggcctgctacagtttgctggttactgttgccttcatcattttttgggtgcgtctgaagattcaggttagaaaggctgcaatcacaagctacgaaaagagcgacggcgtgtatactgggctgtctaccaggaaccaggagacctatgaaactctgaagcacgagaagcccccgcagtctccaaacaggaaaaaccccctgtggccttcagtgccggatcccgctcacagctcactgggatcttgggtccctactatcatggaagaggacgccttccagctgcctggactgggcactccgcctattaccaaactgaccgtgttggaggaagatgagaaaaagccggtaccctgggagagccacaatagttccgaaacttgcggtctgcctaccctggttcaaactcccacgtcttttggttttgacaagccgcatgtgctggtggacttgctggtggatgactccggctaa

SEQ ID NO: 25 - DNA, 1629 bp, synthetic construct
  source 1..1629: mol_type = other DNA
atggctctgccagtcaccgcactgctcctgcctctggctctgcttctgcacgctgcaagacctgatatccagatgacacagtcccctagctccttgagtgcctctgtgggcgaccgcgtgacaatcacctgccgcgcctcccaggacgtgaacaccgccgttgcttggtaccaacagaaaccagggaaagccccaaagcttctgatttactctgctagcttcctgtattctggcgtgccgtctcggttctctggcagtcgctccggaaccgacttcactttgaccatcagctccctccaaccagaggactttgccacttattactgtcagcaacactacaccactcccccaaccttcggtcagggcaccaaggtcgagattaaacgcacgggttccacttccggctctggtaaaccaggcagcggcgagggctccgaagtgcaacttgtggagtccggtggcggtctggtgcagcctggcggatctctgcgcctcagctgcgcggcctccgggttcaatatcaaggacacatacatccactgggtgaggcaagcccctggcaaggggctggagtgggttgcccgcatctacccaaccaatgggtacactcgttacgccgactccgtcaagggtagattcacgatctctgccgatacatctaagaacaccgcatacctccaaatgaattccctgcgtgccgaggataccgctgtctattactgttccagatggggtggcgacggattttacgccatggatgtctggggccagggcaccttggtcactgtgtccagcagtggtattgaagtcatgtacccgccaccgtacctggacaatgagaagtcaaacgggactattatccatgtaaagggaaagcacctgtgcccctctccactcttccctggtcccagtaaaccattctgggtgctggttgtcgtgggtggcgtcctggcctgttattccctcctggtgacagtggcgttcattatcttttgggtgagcccaaaccgcaagaacccactgtggccctctgtgcctgatcctgcgcactccagcctgggctcctgggtgcctactatcatggaagaggacgccttccagctgcctgggctcggaacccctccaatcacgaagctgactgtgctggaggaagacgagaaaaagcctgtgccatgggaatcccataactcttccgaaacctgcggtctgccaactctggttcagacacccacatctttcggttacgataagcctcatgtcctggtagacttgctggtagatgacagtggaagagtgaagttttcccgctcagccgacgctccggcttaccaacagggtcaaaaccagctctacaacgagctgaacctcggccgccgtgaagagtatgacgtgctcgataagcgtcggggccgcgatcccgagatgggaggcaagccgcgccgtaaaaatccgcaggagggtctttacaacgaactgcagaaggacaagatggccgaggcctactccgagatcggcatgaagggcgagcgtaggcgtggcaagggccacgacggcctgtaccagggcctctctacggctacaaaagacacctacgacgctctgcacatgcaagctctgcctccacggtga

SEQ ID NO: 26 - DNA, 1419 bp, synthetic construct
  source 1..1419: mol_type = other DNA
atggctctgcccgtaactgcactgctcctgcctctggccctcctgctccacgcggcccgccccgacattcagatgactcagagcccatccagtctgagtgctagcgtgggtgacagggtcaccattacctgtcgtgcctctcaggacgtgaacacagcggtggcttggtatcaacagaagcccgggaaggctccaaagctgctcatttactcagcctccttcttgtattctggagtgccctcccgctttagcgggtcaaggagcggaacagatttcactctgaccatctcttcactgcagccggaggatttcgccacctattactgccaacagcactataccactcccccaacgtttggccaggggaccaaggtggagattaagagaaccgggtccacttctgggagcggtaagcccggatccggagagggcagcgaagttcagcttgtcgagagcggcggaggcctggttcagccagggggctctctgcgcctgtcatgcgcggcctctggctttaacatcaaggacacctatatccattgggtgcgccaggcacccggtaaaggcctggaatgggtggcgcgcatctaccccactaacggatacacgaggtatgccgactccgttaagggccgcttcaccatcagcgctgataccagcaagaacaccgcctacttgcagatgaatagtctccgggcggaggacacggcagtctattactgtagcaggtggggaggcgatggcttctatgccatggacgtgtggggacagggcactctggtgaccgtatccagcagtggaatcgaggtgatgtaccctcccccttacctggataacgagaaaagtaacggtaccattatccacgtgaagggtaagcatctgtgtccttctcccctgttccccgggccatccaagcccttttgggtcctggttgtggtaggtggggttctggcctgttacagtctcctggtcactgtggccttcatcattttctgggtctctcctaacaggaagaatccgctgtggccaagcgttcctgaccccgctcatagttctctgggctcctgggtgccaaccatcatggaggaagacgcctttcagttgcctggcctgggcactccaccgatcacaaagttgacggtgctcgaagaggacgagaaaaagcccgtcccgtgggagtcacacaattcatccgaaacttgcgggctgcccaccctggtgcagacgccgaccagctttggctacgacaagcctcacgtgcttgtcgacctcttggtcgatgactccggaagactgaaaatccaggttcgcaaagcagctatcacctcatatgagaaaagcgatggcgtctatacaggactgtccacccgtaatcaggagacgtacgaaaccctgaagcacgagaagccaccccagtaa

SEQ ID NO: 27 - protein, 578 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 28 - protein, 825 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 29 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 30 - protein, 521 aa, Homo sapiens
MGLGRCIWEGWTLESEALRRDMGTWLLACICICTCVCLGVSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 31 - protein, 538 aa, Homo sapiens
MPRGWAAPLLLLLLQGGWGCPDLVCYTDYLQTVICILEMWNLHPSTLTLTWQDQYEELKDEATSCSLHRSAHNATHATYTCHMDVFHFMADDIFSVNITDQSGNYSQECGSFLLAESIKPAPPFNVTVTFSGQYNISWRSDYEDPAFYMLKGKLQYELQYRNRGDPWAVSPRRKLISVDSRSVSLLPLEFRKDSSYELQVRAGPMPGSSYQGTWSEWSDPVIFQTQSEELKEGWNPHLLLLLLLVIVFIPAFWSLKTHPLWRLWKKIWAVPSPERFFMPLYKGCSGDFKKWVGAPFTGSSLELGPWSPEVPSTLEVYSCHPPRSPAKRLQLTELQEPAELVESDGVPKPSFWPTAQNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 32 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 33 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 34 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 35 - protein, 422 aa, Homo sapiens
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 36 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 37 - protein, 378 aa, Homo sapiens
MVLLWLTLLLIALPCLLQTKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWRTSLLIALGTLLALVCVFVICRRYLVMQRLFPRIPHMKDPIGDSFQNDKLVVWEAGKAGLEECLVTEVQVVQKT

SEQ ID NO: 38 - protein, 420 aa, Homo sapiens
MIIVAHVLLILLGATEILQADLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

SEQ ID NO: 39 - protein, 400 aa, Homo sapiens
MLLLVTSLLLCELPHPAFLLIPEKSDLRTVAPASSLNVRFDSRTMNLSWDCQENTTFSKCFLTDKKNRVVEPRLSNNECSCTFREICLHEGVTFEVHVNTSQRGFQQKLLYPNSGREGTAAQNFSCFIYNADLMNCTWARGPTAPRDVQYFLYIRNSKRRREIRCPYYIQDSGTHVGCHLDNLSGLTSRNYFLVNGTSREIGIQFFDSLLDTKKIERFNPPSNVTVRCNTTHCLVRWKQPRTYQKLSYLDFQYQLDVHRKNTQPGTENLLINVSGDLENRYNFPSSEPRAKHSVKIRAADVRILNWSSWSEAIEFGSDDGNLGSVYIYVLLIVGTLVCGIVLGFLFKRFLRIQRLFPPVPQIKDKLNDNHEVEDEIIWEEFTPEEGKGYREEVLTVKEIT

SEQ ID NO: 40 - protein, 897 aa, Homo sapiens
MVLAQGLLSMALLALCWERSLAGAEETIPLQTLRCYNDYTSHITCRWADTQDAQRLVNVTLIRRVNEDLLEPVSCDLSDDMPWSACPHPRCVPRRCVIPCQSFVVTDVDYFSFQPDRPLGTRLTVTLTQHVQPPEPRDLQISTDQDHFLLTWSVALGSPQSHWLSPGDLEFEVVYKRLQDSWEDAAILLSNTSQATLGPEHLMPSSTYVARVRTRLAPGSRLSGRPSKWSPEVCWDSQPGDEAQPQNLECFFDGAAVLSCSWEVRKEVASSVSFGLFYKPSPDAGEEECSPVLREGLGSLHTRHHCQIPVPDPATHGQYIVSVQPRRAEKHIKSSVNIQMAPPSLNVTKDGDSYSLRWETMKMRYEHIDHTFEIQYRKDTATWKDSKTETLQNAHSMALPALEPSTRYWARVRVRTSRTGYNGIWSEWSEARSWDTESVLPMWVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 41 - protein, 1097 aa, Homo sapiens
MMDIYVCLKRPSWMVDNKRMRTASNFQWLLSTFILLYLMNQVNSQKKGAPHDLKCVTNNLQVWNCSWKAPSGTGRGTDYEVCIENRSRSCYQLEKTSIKIPALSHGDYEITINSLHDFGSSTSKFTLNEQNVSLIPDTPEILNLSADFSTSTLYLKWNDRGSVFPHRSNVIWEIKVLRKESMELVKLVTHNTTLNGKDTLHHWSWASDMPLECAIHFVEIRCYIDNLHFSGLEEWSDWSPVKNISWIPDSQTKVFPQDKVILVGSDITFCCVSQEKVLSALIGHTNCPLIHLDGENVAIKIRNISVSASSGTNVVFTTEDNIFGTVIFAGYPPDTPQQLNCETHDLKEIICSWNPGRVTALVGPRATSYTLVESFSGKYVRLKRAEAPTNESYQLLFQMLPNQEIYNFTLNAHNPLGRSQSTILVNITEKVYPHTPTSFKVKDINSTAVKLSWHLPGNFAKINFLCEIEIKKSNSVQEQRNVTIKGVENSSYLVALDKLNPYTLYTFRIRCSTETFWKWSKWSNKKQHLTTEASPSKGPDTWREWSSDGKNLIIYWKPLPINEANGKILSYNVSCSSDEETQSLSEIPDPQHKAEIRLDKNDYIISVVAKNSVGSSPPSKIASMEIPNDDLKIEQVVGMGKGILLTWHYDPNMTCDYVIKWCNSSRSEPCLMDWRKVPSNSTETVIESDEFRPGIRYNFFLYGCRNQGYQLLRSMIGYIEELAPIVAPNFTVEDTSADSILVKWEDIPVEELRGFLRGYLFYFGKGERDTSKMRVLESGRSDIKVKNITDISQKTLRIADLQGKTSYHLVLRAYTDGGVGPEKSMYVVTKENSVGLIIAILIPVAVAVIVGVVTSILCYRKREWIKETFYPDIPNPENCKALQFQKSVCEGSSALKTLEMNPCTPNNVEVLETRSAFPKIEDTEIISPVAERPEDRSDAEPENHVVVSYCPPIIEEEIPNPAADEAGGTAQVIYIDVQSMYQPQAKPEEEQENDPVGGAGYKPQMHLPINSTVEDIAAEEDLDKTAGYRPQANVNTWNLVSPDSPRSIDSNSEIVSFGSPCSINSRQFLIPPKDEDSPKSNGGGWSFTNFFQNKPND

SEQ ID NO: 42 - protein, 732 aa, Homo sapiens
MMWTWALWMLPSLCKFSLAALPAKPENISCVYYYRKNLTCTWSPGKETSYTQYTVKRTYAFGEKHDNCTTNSSTSENRASCSFFLPRITIPDNYTIEVEAENGDGVIKSHMTYWRLENIAKTEPPKIFRVKPVLGIKRMIQIEWIKPELAPVSSDLKYTLRFRTVNSTSWMEVNFAKNRKDKNQTYNLTGLQPFTEYVIALRCAVKESKFWSDWSQEKMGMTEEEAPCGLELWRVLKPAEADGRRPVRLLWKKARGAPVLEKTLGYNIWYYPESNTNLTETMNTTNQQLELHLGGESFWVSMISYNSLGKSPVATLRIPAIQEKSFQCIEVMQACVAEDQLVVKWQSSALDVNTWMIEWFPDVDSEPTTLSWESVSQATNWTIQQDKLKPFWCYNISVYPMLHDKVGEPYSIQAYAKEGVPSEGPETKVENIGVKTVTITWKEIPKSERKGIICNYTIFYQAEGGKGFSKTVNSSILQYGLESLKRKTSYIVQVMASTSAGGTNGTSINFKTLSFSVFEIILITSLIGGGLLILIILTVAYGLKKPNKLTHLCWPTVPNPAESSIATWHGDDFKDKLNLKESDDSVNTEDRILKPCSTPSDKLVIDKLVVNFGNVLQEIFTDEARTGQENNLGGEKNGYVTCPFRPDCPLGKSFEELPVSPEIPPRKSQYLRSRMPEGTRPEAKEQLLFSGQSLVPDHLCEEGAPNPYLKNSVTAREFLVSEKLPEHTKGEV

SEQ ID NO: 43 - protein, 372 aa, Homo sapiens
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 44 - protein, 636 aa, Homo sapiens
MRGGRGAPFWLWPLPKLALLPLLWVLFQRTRPQGSAGPLQCYGVGPLGDLNCSWEPLGDLGAPSELHLQSQKYRSNKTQTVAVAAGRSWVAIPREQLTMSDKLLVWGTKAGQPLWPPVFVNLETQMKPNAPRLGPDVDFSEDDPLEATVHWAPPTWPSHKVLICQFHYRRCQEAAWTLLEPELKTIPLTPVEIQDLELATGYKVYGRCRMEKEEDLWGEWSPILSFQTPPSAPKDVWVSGNLCGTPGGEEPLLLWKAPGPCVQVSYKVWFWVGGRELSPEGITCCCSLIPSGAEWARVSAVNATSWEPLTNLSLVCLDSASAPRSVAVSSIAGSTELLVTWQPGPGEPLEHVVDWARDGDPLEKLNWVRLPPGNLSALLPGNFTVGVPYRITVTAVSASGLASASSVWGFREELAPLVGPTLWRLQDAPPGTPAIAWGEVPRHQLRGHLTHYTLCAQSGTSPSVCMNVSGNTQSVTLPDLPWGPCELWVTASTIAGQGPPGPILRLHLPDNTLRWKVLPGILFLWGLFLLGCGLSLATSGRCYHLRHKVLPRWVWEKVPDPANSSSGQPHMEQVPEAQPLGDLPILEVEEMEPPPVMESSQPAQATAPLDSGYEKHFLPTPEELGLLGPPRPQVLA

SEQ ID NO: 45 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 46 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 47 - protein, 622 aa, Homo sapiens
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 48 - protein, 515 aa, Homo sapiens [sequence omitted]

SEQ ID NO: 49 - protein, 489 aa, Homo sapiens
MALLFLLPLVMQGVSRAEMGTADLGPSSVPTPTNVTIESYNMNPIVYWEYQIMPQVPVFTVEVKNYGVKNSEWIDACINISHHYCNISDHVGDPSNSLWVRVKARVGQKESAYAKSEEFAVCRDGKIGPPKLDIRKEEKQIMIDIFHPSVFVNGDEQEVDYDPETTCYIRVYNVYVRMNGSEIQYKILTQKEDDCDEIQCQLAIPVSSLNSQYCVSAEGVLHVWGVTTEKSKEVCITIFNSSIKGSLWIPVVAALLLFLVLSLVFICFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 50 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 51 - protein, 553 aa, Homo sapiens
MRAPGRPALRPLPLPPLLLLLLAAPWGRAVPCVSGGLPKPANITFLSINMKNVLQWTPPEGLQGVKVTYTVQYFIYGQKKWLNKSECRNINRTYCDLSAETSDYEHQYYAKVKAIWGTKCSKWAESGRFYPFLETQIGPPEVALTTDEKSISVVLTAPEKWKRNPEDLPVSMQQIYSNLKYNVSVLNTKSNRTWSQCVTNHTLVLTWLEPNTLYCVHVESFVPGPPRRAQPSEKQCARTLKDQSSEFKAKIIFWYVLPVSITVFLFSVMGYSIYRYIHVGKEKHPANLILIYGNEFDKRFFVPAEKIVINFITLNISDDSKISHQDMSLLGKSSDVSSLNDPQPSGNLRPPQEEEEVKHLGYASHLMEIFCDSEENTEGTSLTQQESLSRTIPPDKTVIEYEYDVRTTDICAGPEEQELSLQEEVSTQGTLLESQAALAVLGPQTLQYSYTPQLQDLDPLAQEHTDSEEGPEEEPSTTLVDWDPQTGRLCIPSLSSFDQDSEGCEPSEGDGLGEEGLLSRLYEEPAPDRPPGENETYLMQFMEEWGLYVQMEN

SEQ ID NO: 52 - protein, 574 aa, Homo sapiens
MRTLLTILTVGSLAAHAPEDPSDLLQHVKFQSSNFENILTWDSGPEGTPDTVYSIEYKTYGERDWVAKKGCQRITRKSCNLTVETGNLTELYYARVTAVSAGGRSATKMTDRFSSLQHTTLKPPDVTCISKVRSIQMIVHPTPTPIRAGDGHRLTLEDIFHDLFYHLELQVNRTYQMHLGGKQREYEFFGLTPDTEFLGTIMICVPTWAKESAPYMCRVKTLPDRTWTYSFSGAFLFSMGFLVAVLCYLSYRYVTKPPAPPNSLNVQRVLTFQPLRFIQEHVLIPVFDLSGPSSLAQPVQYSQIRVSGPREPAGAPQRHSLSEITYLGQPDISILQPSNVPPPQILSPLSYAPNAAPEVGPPSYAPQVTPEAQFPFYAPQAISKVQPSSYAPQATPDSWPPSYGVCMEGSGKDSPTGTLSSPKHLRPKGQLQKEPPAGSCMLGGLSLQEVTSLAMEESQEAKSLHQPLGICTDRTSDPNVLHSGEEGTPQYLKGQLPLLSSVQIEGHPMSLPLQPPSRPCSPSDQGPSPWGLLESLVCPKDEAKSPAPETSDLEQPTELDSLFRGLALTVQWES

SEQ ID NO: 53-56 - skipped (4 entries)

SEQ ID NO: 57 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 58 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 59 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 60 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 61 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 62 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 63 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 64 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 65 - protein, 234 aa, Homo sapiens
MRKTRLWGLLWMLFVSELRAATKLTEEKYELKEGQTLDVKCDYTLEKFASSQKAWQIIRDGEMPKTLACTERPSKNSHPVQVGRIILEDYHDHGLLRVRMVNLQVEDSGLYQCVIYQPPKEPHMLFDRIRLVVTKGFSGTPGSNENSTQNVYKIPPTTTKALCPLYTSPRTVTQAPPKSTADVSTPDSEINLTNVTDIIRVPVFNIVILLAGGFLSKSLVFSVLFAVTLRSFVP

SEQ ID NO: 66 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 67 - protein, 271 aa, Homo sapiens
KTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 68 - protein, 251 aa, Homo sapiens
KWIGYICLRNSLPKVLNFHNFLAWPFPNLPPLEAMDMVEVIYINRKKKVWDYNYDDESDSDTEAAPRTSGGGYTMHGLTVRPLGQASATSTESQLIDPESEEEPDLPEVDVELPTMPKDSPQQLELLSGPCERRKSPLQDPFPEEDYSSTEGSGGRITFNVDLNSVFLRVLDDEDSDDLEAPLMLSSHLEEMVDPEDPDNVQSNHLLASGEGTQPTFPSPSSEGLWSEDAPSDQSDTSESDVDLGDGYIMR

SEQ ID NO: 69 - protein, 223 aa, Homo sapiens
CFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 70 - protein, 12 aa, Homo sapiens
PTSFGYDKPHVL

SEQ ID NO: 71 - protein, 271 aa, Homo sapiens; identical to SEQ ID NO: 67

SEQ ID NO: 72 - protein, 11 aa, Homo sapiens
LAKGYLKQDPL

SEQ ID NO: 73 - protein, 379 aa, Homo sapiens
CYRVNRQQKLSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 74 - protein, 890 aa, Homo sapiens
MALRRSMGRPGLPPLPLPPPPRLGLLLAALASLLLPESAAAGLKLMGAPVKLTVSQGQPVKLNCSVEGMEEPDIQWVKDGAVVQNLDQLYIPVSEQHWIGFLSLKSVERSDAGRYWCQVEDGGETEISQPVWLTVEGVPFFTVEPKDLAVPPNAPFQLSCEAVGPPEPVTIVWWRGTTKIGGPAPSPSVLNVTGVTQSTMFSCEAHNLKGLASSRTATVHLQALPAAPFNITVTKLSSSNASVAWMPGADGRALLQSCTVQVTQAPGGWEVLAVVVPVPPFTCLLRDLVPATNYSLRVRCANALGPSPYADWVPFQTKGLAPASAPQNLHAIRTDSGLILEWEEVIPEAPLEGPLGPYKLSWVQDNGTQDELTVEGTRANLTGWDPQKDLIVRVCVSNAVGCGPWSQPLVVSSHDRAGQQGPPHSRTSWVPVVLGVLTALVTAAALALILLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 75 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 76 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 77 - DNA, 1734 bp, Homo sapiens
  source 1..1734: mol_type = genomic DNA
atgttgccttgcctcgtcgtgctgcttgccgctttgctgagcctccgcctggggtccgacgcccatggtactgagctgccttctcccccttccgtgtggttcgaggccgagtttttccatcacatcctccactggacccccatccccaaccagagtgaatctacatgctacgaagtggcccttctgcgctacggcattgagagctggaactctatctctaactgcagccagacactgtcttatgacctgacggctgtgacactggacctctatcattccaatgggtatcgcgctcgcgtgagggccgtggacgggagccgccacagcaactggactgttaccaatacccgcttctccgtggatgaggtgacacttacagtgggtagtgtcaatctggaaattcacaacggcttcattctgggcaaaatccagctgccccgccctaagatggctccggcgaatgacacctacgagtccatcttttcccactttagagagtacgagatcgctattcgcaaagtgcccggtaacttcaccttcacccacaagaaagtaaagcatgagaatttttctctccttacaagcggcgaagtgggagaattctgcgtgcaggtgaaaccgtccgtggcttctcgttcaaacaaggggatgtggagtaaggaggaatgcatttccctgactcgccagtatttcactgttaccaacgtcattatctttttcgcgtttgtgttgctcctgtctggtgcgcttgcatactgtctggccctccagctgtatgtgagacgcaggaaaaagctgcccagtgtactcctgtttaaaaagccctccccttttatcttcatttcccagaggccttcccccgagacacaggatacgattcaccccctggatgaagaggccttcctgaaggtgtcaccagagctgaagaacctcgacctccacggtagcaccgactccggctttggctccacaaaaccctccctgcaaaccgaagagccccagttcctcctgcctgacccgcacccgcaggctgaccgcaccctgggaaaccgcgagccgcctgtgctgggagattcctgcagctccggcagctctaacagcaccgactctggtatctgtctgcaggagccctccctgagcccttcaacaggcccgacttgggagcaacaggtcgggtccaactctcgtggacaagacgattctggcatcgacctggtccagaactctgagggtcgtgctggagatacacagggtggcagcgctcttggccaccattctcccccggagcccgaggtgcctggcgaagaggatcccgcagccgttgcattccagggatacctgcggcagactaggtgcgccgaagagaaggctaccaagaccggctgtctggaggaagagtctcccctgaccgatggtcttggccctaaattcggtcgctgtctcgtggacgaggccggtctccatccccctgcactggccaagggctacctgaagcaggacccgctggagatgactctggctagcagtggcgcacctactggtcaatggaatcaacctacggaggaatggtccctgttggctctctcctcatgctccgatctcggtatctccgattggagtttcgcccacgatctggcccctctggggtgcgtggccgcaccagggggcctgttgggcagctttaactctgatctggtgaccctgccattgatcagctcccttcagagctctgag

SEQ ID NO: 78 - DNA, 2475 bp, Homo sapiens
  source 1..2475: mol_type = genomic DNA
atgggatggttgtgcagcggccttctgtttcccgtctcttgtctggtactgctccaggtggcttccagcggcaacatgaaggtcctgcaggagcccacctgtgtttctgattatatgtctatctctacttgcgagtggaagatgaacggccccacgaactgttcaaccgaactgcgcttgctctaccagcttgtgtttttgctgtccgaggcgcatacttgtatcccggaaaataacgggggcgcgggctgtgtctgccatttgctgatggatgacgtagtgagtgccgataactatactctggacctgtgggctggccagcaactcctgtggaagggtagtttcaaaccgtccgaacatgttaagccccgcgctcctggcaacctcacagtgcacaccaatgtgtctgacacactgctcctgacttggtccaacccttatccacccgataattacctgtataaccacctcacgtatgctgtaaacatctggtctgagaacgaccccgcggacttccggatttataacgtgacctacctcgaaccctccctgcgtatcgcggcctcaacactgaagagcggtatctcctaccgcgcgcgtgtcagggcgtgggcccaatgttacaacactacctggtctgaatggtctccctcaacaaaatggcacaattcttaccgcgaacccttcgagcagcacctgttgctgggggtgagtgtttcctgcatcgtaatcctggccgtgtgtcttctctgttacgtgagcattaccaagattaaaaaggaatggtgggaccagatccccaaccctgcgaggagccgcctggtggcgatcattatccaagatgcccaaggctcccagtgggagaagcgctcacgtggtcaagagcctgctaagtgcccacactggaagaactgcttgaccaaattgctgccttgcttcctggaacacaacatgaagcgggatgaggacccccataaagcagctaaggaaatgccctttcaaggcagtggcaagtccgcctggtgccctgtagagatctctaagaccgtcctgtggcctgagtctattagcgtagtccgctgtgtggagctgttcgaggcacccgttgaatgtgaagaggaagaggaagttgaagaggaaaagggctcattctgcgcgagtccagagagtagccgcgatgacttccaggaagggcgcgagggtattgtggctcgcctgacggaaagtctgttcctggatctcctgggtgaggaaaatgggggtttctgccaacaggacatgggagaatcttgcttgctgccgccctccggctcaacctccgctcacatgccatgggatgaatttccatccgcgggtcctaaggaggccccaccttgggggaaggagcagcccctgcacctggagccctcaccaccggctagcccaacacagtcacccgacaatctgacttgtacagagactccgctggtgatcgccggcaacccagcctatcggtctttctccaactctctctctcagagcccttgtcctcgcgaactcggccctgaccccctcctggctcgccacctggaggaagtggaaccagagatgccctgcgtgccccaactgtctgagcccacgaccgtcccacagcccgaaccggagacgtgggagcagatcctgcggcgcaacgtgttgcagcacggcgccgctgccgctcccgtgagcgcccctaccagtggctatcaggagttcgtccacgccgttgaacaaggcgggactcaagcatcagcagtagtgggcctcggtcccccaggcgaggctggctacaaggcgttcagctccttgcttgcctcatctgccgtgtctccagagaagtgcggtttcggggcctcctcaggggaagagggctataagcccttccaggatctcatccccggctgccctggcgaccctgcacctgtcccggtaccactgttcaccttcggccttgacagagagcccccacgctccccacagtcctcacacctgccgtcaagcagtcctgaacacctgggcttggagcccggtgagaaggttgaggacatgccaaagcccccgctcccgcaagagcaggctaccgatcccctggtagacagcctgggaagcggcatcgtgtattccgccctgacatgccacttgtgtggacacctcaagcagtgccatggtcaagaggatggcggtcagacacccgtgatggcttctccgtgttgcggatgttgctgtggcgaccgctcttcacccccaaccacaccgctgcgcgcgccggatccgagtcctggcggagtccctctggaggcttccctgtgccccgcgtctctggccccttccggaatttctgagaaaagtaaatcttccagctcctttcacccggcgcccggtaatgcgcagagttcaagccagacacctaagatcgtgaacttcgtaagtgtcggccccacatacatgcgcgtgtct

SEQ ID NO: 79 - DNA, 1377 bp, Homo sapiens
  source 1..1377: mol_type = genomic DNA
atgaccatcctgggcactaccttcggcatggtctttagccttctgcaggtcgtgtccggcgaatctggatatgcccagaacggtgacctcgaggacgcagaactggatgactacagcttctcctgttactctcagctggaagtaaacggttcccagcactccctgacctgcgcgtttgaggacccagacgtcaacatcaccaacctggagttcgagatctgtggcgccctggtggaggtgaagtgcctgaacttccgtaagctccaggagatctacttcatcgaaaccaaaaagttcctgcttatcggaaagagtaacatttgcgtcaaggtgggggagaagtcactcacttgcaaaaagatcgacctgaccacgatcgtgaagccagaggctccattcgacctgtccgtcgtttacagggagggcgccaacgactttgttgtgacctttaacacctcccatctgcagaaaaagtacgtcaaagtgctcatgcatgacgtggcttatcgccaagaaaaggatgagaacaagtggacacatgtgaacctgagttccacgaagcttacacttctgcagcgcaaactgcagcctgccgctatgtatgagattaaggtgcgttccatcccggaccattatttcaagggtttctggagtgagtggagcccctcctactatttcaggacgccggagatcaacaatagcagtggggaaatggaccccatcctcctgaccatctcaatcttgtcattctttagtgtggcacttttggtgatccttgcctgcgtcctgtggaaaaagcgcatcaagccgatcgtctggcctagtctgccagaccacaaaaagactctcgagcatttgtgcaaaaagccccgcaagaacctgaacgtgagtttcaatccagaatcctttctcgattgccagatccacagggtggatgacattcaggcccgcgacgaagtcgagggatttctccaggacacttttccccagcaactggaagagtcagagaagcagcgcctgggtggcgacgtgcagtccccaaactgccccagtgaggacgttgtgatcacacccgagtctttcggcagggacagtagcctgacctgcctggccggaaacgtgtccgcttgtgacgctccgatcctctctagctcccggagcctggactgtcgcgagtccggcaagaacggtcctcacgtgtatcaggacctgctcctgagcctgggtactacgaacagtacactgccccctcccttctccctgcagtccggcatcctgaccctgaatccagtggctcaggggcagccaattctgacctccctgggctcaaatcaggaggaagcttacgtgacaatgtccagcttctatcagaatcag

SEQ ID NO: 80 - DNA, 1563 bp, Homo sapiens
  source 1..1563: mol_type = genomic DNA
atggggctgggtcgctgcatctgggagggttggactctggagtcagaagcattgaggcgcgacatgggcacctggctgcttgcctgtatctgtatctgtacctgtgtctgtctgggcgtgtccgtgacaggggaaggccaggggcctcggtcccgtactttcacctgtctgacaaataacatcctgcgcatcgattgccattggtcagcccctgaattggggcaggggtccagtccgtggcttctgtttacctccaatcaggctccgggtggcacgcacaagtgcatcctccgcggcagtgagtgtaccgtggtccttcccccagaggcagtcctggtgccgtccgataacttcacaattaccttccatcactgtatgagcggccgtgagcaagtgtcactggtcgatcccgaatatctgccacggcgccatgtgaagctggaccctccctccgatctccagtccaacatctcttccggccactgtatcttgacatggagcatctcccctgctctggagcctatgacgaccctgttgtcctatgaactggccttcaaaaagcaggaagaggcttgggagcaggcgcaacaccgtgaccacatcgtgggagtgacctggcttattctcgaggccttcgagctggacccgggtttcatccacgaagcccggctgcgtgtacaaatggctaccctggaggacgatgttgtagaggaagagaggtatacagggcagtggtcagagtggtctcagcctgtgtgttttcaggcgcctcagcggcagggacccctgatcccaccttggggctggcctggcaataccctggtcgctgtgtcaatttttttgctgcttaccggccctacttacttgctctttaagctgagcccccgtgttaaacgcattttctaccagaacgtgccatctcccgctatgtttttccagcccctctacagcgtgcacaacggcaactttcaaacctggatgggcgcccacggcgctggcgtgcttctgtcccaggactgtgccgggaccccacagggtgctttggaaccttgtgtccaggaggcgacagccttgctgacctgtggccctgccagaccctggaaatctgttgccctggaggaagagcaggaggggcctgggactcggctgcctgggaacctgtctagcgaggatgtgctgcccgccggctgcaccgagtggcgcgtccagacactggcgtacctgccccaggaagattgggcccctacttctctgactcgccctgctcccccagattccgagggctcccgctcttccagcagttccagctcttccaataacaataactattgtgccctcggctgttatggcggttggcatctgtcagcgctgcccgggaacacccaaagctccgggcctattcctgctctcgcttgcggactctcctgtgatcatcagggattggaaacgcagcaaggagttgcttgggtcctggctggtcattgccagcgccccggtttgcatgaagacctgcagggcatgttgctgccttccgtgctgtctaaggcgagaagctggacattc

SEQ ID NO: 81 - DNA, 1614 bp, Homo sapiens
  source 1..1614: mol_type = genomic DNA
atgcccaggggatgggccgcgccgctcctgctcctgctcctgcagggcggatggggctgtcctgacctggtctgttacaccgactacttgcagaccgtgatctgcatcctcgagatgtggaacctgcatccatccacccttactctgacttggcaggaccagtacgaagagttgaaggatgaggcaaccagctgtagtttgcaccgttccgcccacaacgccacgcatgctacatatacttgccatatggacgtgtttcacttcatggccgatgacatcttttccgtgaatatcacagatcagtccggtaactattcccaggagtgcgggtccttcttgctggctgagtccatcaagcctgccccacccttcaacgtgaccgttacattctctggtcagtacaacatctcctggcgcagcgattacgaggaccctgcgttttacatgctcaagggtaagctccagtacgagctgcagtatcggaaccgtggtgacccctgggccgtcagcccccgtagaaagttgatcagcgtggactcccgcagcgtgagcttgctgcccctggagttccgcaaggattcttcctatgagctgcaagtccgcgccggcccaatgccaggtagttcctatcaaggcacatggagcgaatggagcgacccagtcattttccagacacagtctgaagagctgaaagagggttggaatccgcacctgttgctccttctgctcctggtgatcgtgtttatcccagctttctggtctctcaagacccatcccctgtggagactgtggaagaaaatctgggccgtccctagcccggagcgctttttcatgcccctgtataagggttgttctggtgacttcaaaaagtgggtaggagcgccttttactggctcttccctggagcttggcccttggagtccagaggtgccctctacactggaggtgtattcttgccacccacctcgctctccagccaagcgccttcaactgaccgaactgcaggagccagccgaactcgtggaatccgacggagtcccaaagccgagtttctggccaaccgctcagaattccggtgggagcgcttactccgaagagagagaccgcccatatggactggtatccattgacacggtgaccgtgcttgatgccgagggaccctgtacatggccttgctcttgtgaggatgacgggtaccctgctctcgacctggatgccggtctggaaccctctcccggtttggaggatccattgctggacgcgggcacaaccgttctctcctgcggctgtgtgtccgctggctcccccggcttgggtggccccctcggaagcctgctcgatcgcctgaagcctccgctcgcggatggtgaggattgggccgggggcctgccatggggaggcaggagccctggaggcgtgtccgagtccgaggccggaagtccacttgcagggctggacatggacacctttgactccggctttgtgggttccgactgctcttcccccgtggagtgtgattttacttctccaggcgacgaagggcctccccgttcttacttgagacagtgggtcgtgatcccaccgcccctgtccagccctggccctcaggcctcc

SEQ ID NO: 82 - DNA, 1653 bp, Homo sapiens
  source 1..1653: mol_type = genomic DNA
atggccgcaccagctctttcctggcgcctccctctgctcattcttctgttgccactggctacatcctgggccagtgctgcagtgaacggtaccagccagtttacctgtttctacaactcccgcgctaacatttcttgtgtgtggagtcaggacggggcgctgcaggatacctcctgccaggttcacgcctggcccgatcgccgtcgctggaaccagacttgcgaattgctgccggtgagccaagcctcctgggcctgtaatctgattctcggtgcccccgattctcagaagctgactaccgtcgatattgttaccttgcgtgttctgtgtagagaaggagtgcgctggcgcgtgatggctattcaggactttaaacccttcgagaacctgaggctcatggcacccatctcacttcaggtagtgcatgtggagactcaccgctgcaatatttcctgggagatctcccaggcgtcccattacttcgagcgtcacctcgaatttgaggctcgtaccctgagcccgggacacacatgggaagaggctccactcctgactttgaagcagaaacaggagtggatctgcctggagaccctgacccccgacacacagtatgagttccaggtgcgcgtgaagccccttcagggcgagttcactacctggtctccgtggagccagcccttggcattccgtacgaagccagcggctctgggaaaggatacgattccttggctgggtcatctgctcgtgggcctctccggggccttcggttttattatcctggtctacttgctgattaactgccggaacactggaccttggctgaagaaagtgctcaaatgtaatacacccgacccaagcaagttcttttctcagctgtcttccgagcatggaggcgatgtgcagaagtggttgtcctctcccttcccttctagctccttcagcccgggcggactggcgccggagatctctcccctggaggtcctggagcgcgataaagtgacccaactgctcctgcaacaggacaaggtgcccgagcctgctagcctctcttcaaaccactccctgacctcctgttttacgaaccagggctatttctttttccatttgcccgatgctttggagatcgaagcctgtcaggtgtatttcacctatgacccgtactctgaagaggatcccgatgaaggcgtggccggtgctccaaccggaagcagtccccagccactgcagcctctgtccggtgaagatgacgcttactgtaccttccccagtagggatgacttgctcctgttcagtcccagcctcctgggtggacccagccctccctctaccgcgcctggagggtccggcgccggtgaagagcgcatgccgccttccctgcaagagagagtgccacgcgactgggatccccagccactcggtcccccaactcctggcgtgcccgacctggtcgatttccagccacctccggagttggtcctccgtgaagctggcgaagaggtgccagacgctggtcctagggagggggtcagctttccatggagccgcccacccggtcagggggagttccgcgctcttaacgctcggctgcccttgaacacagatgcttacctttccctccaggagctgcaaggacaagatccgacccacctggtg

SEQ ID NO: 83 - DNA, 2754 bp, Homo sapiens
  source 1..2754: mol_type = genomic DNA
atgctgaccttgcagacttggctggtgcaggctctgtttatcttcttgacaaccgaatctactggagaactcctggatccctgtggctacatctctcccgaaagtcccgttgtgcagcttcattccaatttcactgcggtgtgtgtgcttaaggagaagtgtatggattacttccacgtgaacgcgaactacatcgtatggaagacaaaccacttcaccattcctaaggagcagtacacgattatcaatagaacggcatccagcgtgacattcaccgacatcgcgtccctgaatattcagctgacctgtaatatcctcaccttcggccagcttgaacagaacgtgtacggcatcactattatcagtgggctgccccctgaaaagcccaaaaatctgtcttgcattgtgaacgaggggaaaaagatgcgctgcgaatgggacggtggccgtgagacacatctcgagacgaattttaccttgaagtccgagtgggccacccacaagttcgccgactgtaaggctaaacgtgacacccccacctcctgcacagtggattacagcaccgtctacttcgtgaacatcgaagtctgggtggaggcggagaacgcgctgggtaaggtgacctctgaccacatcaactttgaccccgtgtataaggtgaagcccaacccacctcacaatctttccgttatcaactccgaagagctctcttccattcttaagttgacctggacaaacccttccatcaagagcgtgatcattctgaaatacaacatccagtatcgcacaaaggacgcctccacttggagccagatccctccggaggacaccgcgagtacccgcagctctttcacagtccaggaccttaaaccgtttactgagtatgtgttccgcatcaggtgcatgaaggaggacggcaagggatactggagcgactggtccgaagaggctagcggtattacatacgaggaccgtccttctaaggccccttctttttggtacaagatcgaccccagccatacacagggttatcggaccgtccagttggtgtggaagaccctgcctcccttcgaagccaacggtaagatcctggactacgaggttaccctgacccggtggaagtctcacctgcagaactacaccgtcaatgcaacaaagctgacagtgaacctgaccaacgaccgctatctcgcaactctgacagtgcgcaacctggtcggtaaaagtgatgctgccgtactgactatcccagcgtgtgactttcaggcaacccatccagtcatggacctgaaggccttcccaaaggataacatgctgtgggttgagtggaccactccgagggaaagtgttaaaaagtacatcctggagtggtgcgtgctgtctgacaaggccccctgcatcaccgactggcagcaagaagatggcactgtacaccgtacttacctccgcgggaaccttgccgaatcaaaatgttacctgatcactgtgacccccgtgtacgcagacggtcccggtagtcccgaatcaattaaggcctacctcaagcaggctccaccgtccaagggccccactgtccgtaccaaaaaggtgggtaaaaacgaagctgtgctggagtgggaccagctgccggtggacgtgcagaacggctttattcgtaactacacaattttctatcgcacgattatcggaaatgaaacagcggtgaacgtggactccagccacaccgagtacaccctcagctccttgaccagtgacacgctctacatggttcggatggctgcctataccgatgaaggaggtaaagatggacctgagttcacctttacgacccccaagtttgcacagggcgaaattgaagcaatcgtcgtgccagtctgcctcgccttcctcctgaccactctcctgggggtcctgttttgtttcaacaagcgggatctgatcaaaaagcatatctggccaaatgttcccgaccctagtaagtcccatattgctcagtggtctccccacacccctcccaggcacaattttaactccaaggaccaaatgtactcagacggcaacttcaccgatgtatcagttgtggagatcgaggctaacgacaagaaacctttccccgaggacctcaaatccctggatctgtttaagaaagagaagatcaacacagaaggtcattcttccggaatcggtggctccagctgcatgagctcaagccgcccaagcatcagctccagcgacgagaacgagagcagtcagaacacatccagcaccgttcagtattccactgtagtccattctggataccgtcaccaggtccccagcgtgcaagtgttctccaggagcgagtccacccagcccctgctcgattcagaagagcggccagaggatctgcagctggtcgaccacgtggacgggggtgatggtatcctgccaaggcaacagtacttcaagcaaaattgctcccaacacgaatcttccccggacatctcccatttcgagagatctaagcaggtctccagcgtcaacgaagaggactttgtgaggctgaaacaacagatttccgatcatatcagccagagctgcggttccggacagatgaaaatgttccaggaggtgtctgctgccgacgcgtttggccccgggaccgaaggacaagtcgagaggttcgagaccgttggtatggaggccgctacagacgagggcatgccgaaaagctacctgccccagaccgtgcgccaaggtggctacatgcctcaa

SEQ ID NO: 84 - DNA, 1404 bp, Homo sapiens
  source 1..1404: mol_type = genomic DNA
atgctggcggtgggctgcgctttgctggcggccctgctcgctgcccctggagcggccctggctccgcgtcgctgccccgctcaggaggtcgcgcgtggagtgttgacttctctgcctggcgatagcgtgactttgacttgccctggcgtagagcctgaggataacgctaccgtgcattgggtactgagaaagcccgctgccggctcccatccttcccgctgggcaggcatggggaggcgcctgctcctgcggtcagttcaattgcacgactctggtaattacagctgctaccgggccggacgcccagccggcaccgtccatctgctcgtcgatgtgccgccagaggaaccacagctctcctgttttaggaagtctccactttccaacgttgtgtgtgagtggggcccccggagcacccctagcttgacaactaaggccgtgctgctcgtgcgcaaatttcagaactcccccgccgaagactttcaggagccgtgccagtacagccaggaatcccagaagttcagttgccagctggccgttccagagggtgactccagcttctacattgtgagtatgtgcgtggcgagttcagtcggctcaaagttttcaaagacccagacttttcagggatgtggtattctccagccggatcctcccgccaacatcactgtcaccgccgtggcacggaaccctcgctggctgagcgtcacatggcaggacccccactcctggaacagtagcttttaccgtctccgcttcgagctccgttatagagctgagcgctccaagaccttcaccacatggatggtaaaggacctgcagcatcactgtgtcatccacgacgcctggtctggactgcgtcatgttgtgcagctcagggcccaggaagagttcgggcagggggagtggtccgaatggtctccggaggccatgggcacaccatggactgagagcagatccccacccgctgaaaacgaggtttctaccccaatgcaggcgctgaccactaacaaagacgatgacaatatcctgttccgcgatagtgccaacgcgacctccctgcccgtgcaggactcctcttccgtgccactcccaacgtttctggtggccggaggctctttggccttcgggactcttctgtgcatcgcgattgtgttgcgcttcaaaaagacatggaagttgcgcgcgctcaaggagggcaagacttctatgcatccaccttattctctgggtcaattggtgcctgagaggcctagacccacccccgtgctggtccccttgatttcccctcccgtctccccaagctcactgggaagcgacaacactagttcccacaacaggccagatgcacgggatccccgttcaccgtacgacatctcaaacaccgactattttttccctaga

SEQ ID NO: 85 - DNA, 1266 bp, Homo sapiens
  source 1..1266: mol_type = genomic DNA
atgagctcctcttgctccggtctctcccgcgtgctcgtggccgttgctacagctctggtgtctgccagctctccttgtcctcaggcgtgggggccccctggcgtgcagtacggacagcccggccgctctgtgaagctgtgttgccccggcgtgacagcaggcgatcctgtgagttggtttagagacggagagccaaaactcctgcagggacctgactccggtctggggcatgagttggtcctggcccaggccgatagtacggacgaggggacctacatctgccagactctcgacggcgccctgggaggcacggtgacgctgcagctgggttaccctccagctagacccgtggtctcctgtcaggccgctgactatgagaatttcagttgtacatggagccccagtcagatttcaggcttgcccactcgctacctgacttcctacagaaaaaagacagtcctgggagcagacagccagcgcaggtcacccagtaccggaccttggccgtgccctcaggaccctttgggtgctgcccgttgcgttgtgcatggcgccgagttctggtctcagtataggatcaatgtaaccgaagtgaaccctctgggtgcgtccacccggctgctcgacgtttctttgcagagcatcctgcgccccgacccgccccaaggacttcgcgtggagtctgtccccggctaccctcgtcggctgcgcgcgagttggacttacccggccagctggccctgccaaccccactttctcctgaaatttcgtctgcagtatcgccccgctcagcaccctgcctggtctactgtggaacccgcgggactggaagaggttatcacagacgccgtcgctggcctgccccatgctgtgcgcgtttcagcccgcgacttcctggacgccgggacctggagcacctggtccccagaggcttggggcactccaagcaccggcacaatccctaaggagatccctgcttggggccagctgcacactcagccagaggtggagcctcaggttgactctcccgctccccctcgccctagcctgcagcctcaccccaggctcttggaccaccgcgatagcgtcgagcaggtggcagtgctggcgtctctggggatcctttcctttctcggcctggtcgccggcgctctggcactggggctctggctcaggctcaggcgtggtggcaaggacggctcccctaagcctggcttcctggccagtgtgatccctgtggatcgcaggcccggagcgcctaatttg

SEQ ID NO: 86 - DNA, 2508 bp, Homo sapiens
  source 1..2508: mol_type = genomic DNA
atggccagactgggcaactgttccctgacatgggccgcactgattatcctgctcctgccgggcagcctggaagagtgcggccacatctctgtgtcagcccccatcgtgcacctgggtgacccgatcaccgcttcttgcattatcaagcagaactgttcccacctggaccccgaacctcagattttgtggcgcctgggcgccgaattgcagccaggtggccgccaacagcgcctgtccgatggcacccaggagagcatcattacccttccccaccttaatcatacacaggccttcctgtcttgttgcctgaactggggtaattccctgcaaatcctggatcaggtggagctgcgcgccggctacccgcctgcaatccctcataatctgagctgtctgatgaatttgactaccagcagtctgatctgtcagtgggagccgggcccggaaactcatcttccgacttcatttacactgaaatcctttaaaagccgcggcaactgtcagactcagggagattccatcctggattgcgtcccaaaggacggccagagccactgctgtattcctcgcaagcacctgcttctgtaccaaaacatgggtatctgggtccaggcagagaacgccctggggacatccatgtctccccagctgtgtctggaccccatggatgttgtgaaattggagcccccaatgctgcgtaccatggaccctagccccgaggctgcgccaccccaggccggctgtctgcagctgtgttgggagccgtggcaacccggtcttcacatcaaccagaagtgtgaactccgccacaagccccagcgtggagaagcaagctgggctctggtgggccccctccctctggaggccctgcagtacgagctgtgtgggcttctgcctgcgacagcttataccctgcagatccgctgtatccgttggcccctgccgggccactggtccgactggtccccctccctggagctgaggactaccgagcgtgcgcctactgtccgcctggatacctggtggcgtcagcgccagctcgaccctcgcaccgtccagctgttctggaaaccagtgccgctggaggaagatagcggacgcatccaggggtatgtcgtgtcttggcggccctccggacaggctggggcgatcctgcccctctgtaacaccacggagttgagttgtaccttccatctcccttctgaggcccaggaagtggcgttggtggcctacaactcagcaggcaccagtagacccacacccgtggtcttttctgagagccggggaccagctctgactcggctccatgcgatggcacgggatccacattccctgtgggtggggtgggagccccctaatccttggccgcagggctacgtgatcgaatgggggcttggacccccatccgcatccaattccaacaagacctggcggatggagcagaatggtagagcaaccgggttcctcctgaaggaaaatattaggccgttccaactctacgaaattatcgtcacccctctgtaccaagacactatgggtcccagccagcacgtgtatgcgtactcccaggagatggccccctcacacgccccagaacttcacttgaagcacatcggtaagacatgggcccagctggagtgggtaccagagccgcccgagctcggtaaaagccctctgacacactacactatcttctggaccaacgcccagaaccagtccttctctgccattctgaacgcgtcttcccgcggctttgtgctgcacggcctcgagcccgcgagcctgtaccacattcacctcatggcagctagccaagccggcgcaaccaactccaccgttctgaccctgatgaccctgacccccgagggctcagaactgcacattatcctgggcctctttggattgctgctcttgctgacctgtctgtgcggcactgcatggctctgctgttcccctaaccgcaagaacccactgtggccttctgtgcccgatccagcgcattccagtctgggctcctgggtgccgaccatcatggaagaggacgcctttcagctcccaggtctggggacgcccccgattacaaagctgactgttctggaagaggacgagaaaaagcctgtgccatgggagtcccacaattcctctgaaacttgtggtctgcctaccctggtgcagacctacgtgctgcagggggacccaagagctgtttctacccagcctcagtcccagagcggcacatctgatcaggtgttgtatggccaacttctcggtagcccgactagccccggccctggacattacttgcggtgtgacagcacccagccattgctggccggcctgacaccatccccaaagtcctatgagaacctctggtttcaggcttctccactcggcaccctggtcaccccggcccccagccaggaagatgactgtgtctttggccccctcctgaacttcccccttctgcagggaatccgcgtccacggtatggaggccttgggttccttc

SEQ ID NO: 87 - DNA, 1134 bp, Homo sapiens
  source 1..1134: mol_type = genomic DNA
atggtgctgctttggttgaccctgctcctgattgccctgccatgcctcctgcagacgaaggaggatcccaacccgcccatcaccaacctgagaatgaaggcgaaggctcaacagctcacttgggacctgaaccgtaacgtgaccgacatcgagtgcgtgaaagatgccgattactccatgccagcagtcaataactcttattgtcagttcggtgccatcagcttgtgtgaggtgactaattacaccgtgcgcgtggcgaacccacccttcagtacctggatcctgttccccgagaatagcggtaagccctgggccggcgccgagaacctgacgtgctggatccatgacgtggactttctgagctgttcctgggccgtgggcccaggcgccccagccgatgtacagtacgacctgtatctgaacgtcgccaataggcgccagcaatatgagtgccttcactataagaccgacgcacaaggtacccgtattggctgtcgctttgatgacatttccagactgagctccggctcccagagctctcatatcctggttcgggggcgctccgctgcatttggtatcccctgtaccgataaatttgttgtgttcagccagattgagattctgactccccctaacatgacggcgaaatgcaacaagacacattcatttatgcactggaagatgcggtcccattttaaccgcaaatttcggtatgaactccagattcagaagcgtatgcagcccgtcatcaccgagcaggtgcgcgatcgcaccagtttccagcttctgaacccgggcacctatacagtgcaaatcagagcaagagaacgcgtatacgaattcctgtctgcgtggtctacccctcagcgttttgagtgcgaccaagaggaaggtgccaacacacgcgcttggcgcaccagcctcttgattgccctgggcacacttctggccctggtgtgtgtgtttgtgatctgccgcagatacctcgtgatgcagcgtctgttcccccgtatccctcacatgaaggacccgatcggcgactcctttcagaacgataagcttgtagtgtgggaggccggtaaggccggtttggaagagtgcctggtgaccgaagtccaggttgtgcaaaaaact

SEQ ID NO: 88 - DNA, 1260 bp, Homo sapiens
  source 1..1260: mol_type = genomic DNA
atgattatcgtggcccacgtgttgctgatcctgctcggtgcgactgaaattcttcaggccgatcttctccctgatgagaaaatctcacttctgccgcccgtaaactttacaatcaaggtcacaggcctggctcaagtacttctgcaatggaaaccaaaccccgaccaagaacagcggaatgtgaacctggagtatcaagtgaagattaacgcccccaaggaggacgattacgaaacccgcatcacagagtccaagtgcgtcaccatcctgcacaagggctttagcgcgagtgtccgtaccatcctgcagaacgaccatagcctcctggcctctagttgggccagtgctgagctgcacgctccccctggatccccaggtaccagcatcgtgaaccttacctgcactaccaataccactgaggacaactatagcaggctccgctcttaccaggtatctctgcactgcacttggctggtgggcacagacgcgcccgaggacacccagtattttttgtattaccgttacggctcttggactgaagagtgccaggagtactctaaggatactctgggccgtaatatcgcatgctggttccctcgcacttttatcttgtccaaagggcgggattggttggccgtgctggtgaatggctccagcaagcactccgctatccgcccattcgatcagctgttcgctctgcacgcgatcgaccagatcaatcccccgctgaacgtgaccgctgagatcgagggcactcggctgtctatccagtgggagaaaccggtgagtgccttccccatccactgtttcgactacgaggtgaagattcacaacaccagaaacggctatctgcagatcgagaagctgatgaccaacgcctttatttccattatcgatgacttgtccaaatacgacgtgcaggtgcgcgcggctgtcagctctatgtgccgcgaggccgggttgtggagtgagtggagccagcccatctacgtgggcaacgacgagcacaagcctcttcgggaatggttcgtgatcgtcatcatggccaccatctgtttcatcttgctgatcctcagtctgatctgcaagatttgccatctctggatcaagctctttccccctatccccgcccccaaatctaacatcaaggatctttttgtgacaaccaactatgagaaagctggttccagcgaaaccgaaattgaggtgatctgctacattgaaaaacctggagtggagacgttggaagactccgttttt

SEQ ID NO: 89 - DNA, 1200 bp, Homo sapiens
  source 1..1200: mol_type = genomic DNA
atgctgcttctcgtgacctccctgctcctgtgtgagctgccgcaccctgcctttctcctgatcccagagaagagcgacctgcggaccgttgccccggcctcctctctgaacgtaagattcgacagccggaccatgaacctcagctgggattgccaggaaaacacaaccttctccaagtgcttcctgactgacaaaaagaatcgcgtagtggaacctcgcctgtccaataacgagtgctcctgtaccttccgcgagatctgtctgcacgagggtgtgacctttgaagtccacgtcaacactagccagagagggttccaacagaagttgctgtacccgaactctggacgggagggcaccgctgcccagaatttctcatgctttatctacaacgctgaccttatgaactgcacctgggcacgtggccccaccgcgccccgtgacgtccagtacttcctctacatccgcaattccaagagacggcgtgagattaggtgtccatattacatccaggactctggcacccatgtggggtgccatctggataacctgagcggccttacctctcgcaactactttctcgttaatggcacgagcagagagatcggcatccagtttttcgattctcttctggacacaaagaaaatcgaaaggttcaacccaccctctaacgtcaccgttcgctgtaacactacccattgtttggtgaggtggaaacagccacgcacctaccagaagctgtcatatctggacttccagtaccagctggacgtccatcgcaaaaatacacagcctggcaccgaaaacctgcttatcaacgtgtccggcgatctggagaaccgctacaacttccctagttctgagccgcgcgctaaacacagtgtcaaaatcagagctgccgacgtgaggattctcaactggagttcctggagtgaagcaatcgagttcggctcagatgacggtaacctgggctctgtgtatatctacgtcctgcttatcgtgggtacactggtttgtggcattgttctgggtttccttttcaagaggttcctgagaatccagaggctgtttcccccagtgccgcaaatcaaagataagctcaacgacaaccacgaggtggaagatgagattatctgggaggaattcactcctgaagagggaaaaggctatagggaggaagttctgaccgtgaaggagattact

SEQ ID NO: 90 - DNA, 2691 bp, Homo sapiens
  source 1..2691: mol_type = genomic DNA
atggtcctggcacagggactcctttccatggccctgcttgctctttgttgggagcgcagcttggccggcgccgaagagaccatccctttgcagaccctgcgctgttataatgactacacttcacacatcacatgtcgctgggccgacacgcaggacgctcaacgcctcgtgaacgtgaccctcattcggcgcgtgaacgaggacctgcttgagcctgtgtcatgtgatcttagcgatgacatgccctggtccgcctgtccacacccgcggtgtgtaccgcggcgctgcgtcatcccttgtcagtccttcgtcgtgactgacgtggactacttttccttccaacctgacagacccctgggtactcgcctgacggttactctgacccagcacgtgcagccacccgaacctcgcgacctgcagatctctaccgaccaagaccactttcttctgacttggtccgtggctcttgggagccctcaaagtcattggctcagccccggtgacctggagttcgaggtggtttacaagcgtcttcaggattcttgggaggatgcggctatcctgctctccaacaccagccaggctacccttggccccgagcacctgatgccatcatccacctatgtggcccgcgtgaggacgcgcctggcacctggcagtcgtctgtctggtagaccctccaagtggagtcctgaggtctgttgggacagccagcctggcgatgaagctcagcctcagaacctggagtgctttttcgacggcgcggctgtgctgtcttgtagctgggaggtgcgcaaagaagttgctagctctgtgtccttcggccttttctataaaccctctcctgacgccggtgaagaggaatgctctcctgtgctgcgtgagggcctgggctccctgcatacccgtcaccattgccagattcctgtaccggacccagccacccacggccaatatattgtgagtgtgcagccccgtcgcgccgaaaaacatattaagagctccgtgaacattcagatggctccgccttccctgaatgtgaccaaggacggcgacagctacagcctgcgctgggagaccatgaaaatgcgttacgagcacattgaccacacttttgaaattcaatataggaaggacaccgcaacctggaaggactctaagactgagactctgcagaacgcccactcaatggccctgcccgcgcttgagccaagcaccagatactgggctcgtgtgcgtgtgcgcaccagtcgcacaggttacaacggcatctggagcgaatggagcgaggctcgtagctgggacaccgagtccgtcctgccgatgtgggtcctggccctgatcgtcatcttcctcaccatcgccgtcttgctggccctgcgtttctgtgggatttacggatatcgtctgcggagaaaatgggaggaaaagatccctaacccgagcaaatcccacctgttccagaacgggagcgccgagctgtggccgcccggttctatgtccgctttcacctcaggcagcccaccccaccagggcccatgggggtctcggttccctgagttggagggcgtcttccccgtagggttcggcgatagcgaggtgagtcctctgacaatcgaggatccgaaacacgtgtgtgaccctccatctggccccgacaccacacccgcggcctccgaccttcctacagagcagccccctagcccccagcccggtcctcccgctgcctcccatacacccgagaagcaggctagctcttttgatttcaacgggccttacctgggaccaccccattccaggtctctgccagatatcctgggtcagccagaaccaccgcaggagggagggtcccagaagtctcccccgcccggatccctggagtacctttgtctgccggcaggaggccaggttcagctggtgcctctggcccaggccatggggcccgggcaggcagttgaagtggagcggcgtccaagtcaaggtgcggccggcagtccgtctctggagtccggagggggcccggccccaccggccctgggccccagggtcggcggtcaggaccagaaggactcccccgtagccattcccatgtccagcggcgatacagaggatcccggtgttgcctcaggatacgtttccagcgcggatctcgtgtttacccctaattccggcgcttcaagcgtctctctggtgccttctcttggtctgccgagcgaccagacgcccagcctgtgtcccggtctggcttctggaccccctggcgcccccggccctgtcaagagcggttttgagggctacgtcgagctgcctccgatcgaagggcgctctccccgctctcctcgcaataaccccgtgcctcccgaggcgaaatcacctgtgctgaacccaggtgaacgccctgccgatgtctcccctacctcaccccagccggagggactcctggtgcttcaacaggtgggtgactattgtttccttccagggctgggccctggacccctgtctctgcgttcaaagccttccagccctggccctggccctgaaattaagaacctggatcaggcatttcaggtcaaaaagcctcccggccaggccgttcctcaggtgccggttattcaactgttcaaggccctgaagcaacaggattacctgtctctgcccccatgggaggtgaacaagcccggcgaggtttgt

SEQ ID NO: 91 - DNA, 3291 bp, Homo sapiens
  source 1..3291: mol_type = genomic DNA
atgatggatatttatgtgtgcctcaaacgcccctcatggatggtcgataacaagaggatgaggaccgctagtaactttcagtggctcctgagcaccttcatcctgctctacctcatgaaccaggtgaactcccagaaaaagggagcgcctcacgacctgaagtgtgtgacaaacaatctgcaggtctggaattgcagctggaaggctccttccggcactggccgtgggacggactacgaggtttgcatcgaaaatagaagccgctcttgctatcagttggagaagaccagcatcaagatcccggccctgtcccatggcgactacgagatcactatcaacagcctgcatgatttcgggagctccacgtccaagtttactctcaatgagcagaacgtgtcccttatccctgatactcctgagattttgaatttgagcgctgacttctcaacctctacgctgtatttgaagtggaacgatcgcggctccgtgttcccgcatcgcagtaacgtgatctgggagatcaaggtcctgcgtaaggaaagtatggagctggtgaagctggtgacacataatactaccttgaacggtaaggacaccttgcatcactggtcttgggcgagtgacatgcctctcgaatgcgccatccactttgtggagatcaggtgctacattgacaacctgcatttctccggtctggaagagtggagtgactggtcccccgtgaagaatatctcctggattccagattcccagacaaaggtgtttcctcaggacaaggtcatcctggtgggctccgatatcaccttttgttgcgtgtctcaagagaaagtgctctccgctttgattggacatacaaactgccctctgattcacttggatggcgagaacgtggccatcaagatccgcaacatctccgtgagcgctagttcaggaaccaacgtggtcttcacaaccgaggacaatatctttggcactgtgatctttgcgggctatccacccgacacaccccaacagctgaattgtgagactcatgatctgaaggaaattatctgttcctggaaccctggacgcgtcaccgccctggtcggccctcgcgcgacctcatacactctggtagaatctttttctggaaagtacgtgcgcttgaagcgtgccgaggccccaactaacgaatcataccagctcctgttccagatgcttcccaatcaggaaatttataacttcacactcaatgcccacaaccctctcggcagatcccagagcaccatcctggtgaacattacagagaaagtgtaccctcacacacccacaagtttcaaggtgaaggacatcaactctacggcggtaaaactgtcttggcatctgcctggaaacttcgctaagatcaacttcctgtgtgagatcgaaatcaaaaagagcaatagcgtgcaggagcagaggaacgtcaccatcaaaggtgtggagaactcctcttacttggtcgcattggataagttgaatccttacacactgtacacattcaggattagatgttccacggagactttctggaagtggtctaaatggagcaacaaaaagcaacacctcacaaccgaggccagcccgtctaagggccctgacacgtggcgggagtggtcctcagacggcaagaacctcatcatttattggaagccgctgcctattaacgaggcaaatgggaagattttgtcttacaatgtcagctgctcatccgacgaagagacccagtccctcagcgagatccccgacccacagcataaggccgagattcgtttggacaagaacgactacattatctcagtcgtggctaagaactccgtggggtccagcccgccctctaagatcgccagcatggagatccccaacgatgacttgaagatcgagcaagtggttggcatgggcaagggcatcttgctcacctggcattatgatccaaacatgacatgcgattacgtcattaagtggtgcaattctagccggtccgagccttgtttgatggactggaggaaagtgcccagtaactcaaccgagacagtgatcgagtctgacgaattccgcccagggattcgttacaactttttcctttatggttgccgcaatcagggctatcaacttctgcgcagtatgattggatacattgaagagctcgcccccatcgtcgcacctaatttcaccgtcgaggacacaagtgcagacagcatcctcgtcaagtgggaggacattccggtggaagagctccggggatttctcaggggttatctcttctatttcggcaagggcgagcgcgacacttctaagatgcgcgtactcgagagcggccgtagcgatatcaaggttaagaatattacggacatctcacaaaaaaccctgaggatcgccgacttgcaaggcaagacttcttaccatctggtgcttcgcgcttacaccgacggcggtgtgggccctgagaagtctatgtacgtggttacgaaggagaactccgtgggcctgattatcgctatcctgattcctgtggctgtggcagtgatcgtgggtgtggtcacaagcattctgtgctaccggaagagagagtggatcaaggagacattttatcccgatattccgaaccccgagaactgtaaggccctgcagtttcagaagtccgtatgtgagggcagttctgctctgaagaccttggaaatgaacccatgtacgcccaataacgttgaagtactggagactcgctccgccttcccaaagatcgaggataccgagattatctccccagtggccgaacgtcccgaggaccgctctgatgcagaaccggagaaccacgtcgtggtttcatactgtcccccgattatcgaagaggaaatcccgaaccccgctgccgacgaggccggtggcacagcccaagtgatctacatcgacgtgcagagcatgtatcagccccaggccaaacccgaggaagagcaggaaaatgaccccgtcggcggtgcgggctataagccccagatgcacctgcccatcaactccacagttgaagacatcgccgctgaagaggacttggacaagaccgcaggctatcgtcctcaggcaaacgtgaacacctggaaccttgtaagccctgacagtcctcgcagcatcgactcaaactccgagatcgtttctttcggctctccgtgttccattaactcccggcagtttctgattccccctaaggacgaagattcccccaaatctaacggtggcggatggtccttcaccaactttttccagaacaaaccaaacgat

SEQ ID NO: 92 - DNA, 2196 bp, Homo sapiens
  source 1..2196: mol_type = genomic DNA
atgatgtggacgtgggccttgtggatgctgcccagcctgtgtaagttcagcctcgctgccttgccagccaagcctgaaaacatcagttgtgtctattactatcgtaagaacctgacttgcacttggagcccgggaaaagagaccagctatacccagtacaccgtgaagcgcacatatgcttttggagagaagcatgataattgtacaactaactctagcacctctgaaaacagggccagttgttcttttttcctgccccgtattaccatccctgacaactacaccattgaagtggaagccgagaatggcgacggcgtgatcaagagtcacatgacctattggagacttgaaaatatcgccaagactgaaccgcctaagatttttcgtgtgaagcccgtgctggggatcaagcgcatgattcagattgaatggatcaagcctgagctggccccagtgagctctgacctgaaatacactctgcgcttcagaactgttaactctacctcctggatggaagttaactttgccaagaaccgtaaggacaagaaccagacatataacctgacgggcctgcagccttttacagagtacgtcatcgccctgcgttgcgctgtgaaagagagcaagttctggtctgactggtcccaggagaagatgggaatgactgaagaggaagccccttgcggtctggagttgtggcgcgtgctgaaacccgctgaggccgacggcagacgccccgtgcgtttgctgtggaaaaaggcgaggggagctcctgttctggaaaaaacactgggatacaacatctggtattaccctgaatctaacactaatctgaccgagaccatgaacacaaccaatcaacagctggagctgcatctcggaggcgagtccttctgggtgtccatgatcagttacaatagcctcggcaagtcccccgtggccacgctgcggatccccgctatccaggagaagagctttcagtgcatcgaggtcatgcaggcctgcgtggcggaagaccagctggtggttaaatggcagtcctctgccctggacgtcaacacctggatgatcgagtggtttcctgacgtcgactccgaacccacgacactctcttgggagtccgtttcccaagccacaaactggaccatccagcaagacaaactgaaacccttctggtgttataacatttccgtgtatcctatgctgcacgacaaggtaggggagccatactccatccaagcctacgccaaagagggggttccgtcagaaggccccgagactaaagtggaaaatatcggcgtgaaaacagtcaccattacctggaaggagatccccaagtccgagcgcaaaggcatcatttgcaattatacaattttctatcaggccgaagggggaaagggcttctccaagaccgtgaacagtagcattctgcagtacggactggaatccctgaaacgcaagacctcctatattgtgcaggtgatggcctccaccagcgccggcggtacaaacggcacctcaatcaatttcaagactctgtccttctccgtatttgagattatcttgattacttccctgatcgggggtggccttctgattctcattatcctcaccgtagcatacggcctgaaaaagcctaataagctgacccacctctgttggccaaccgtgccaaaccccgctgagagtagcatcgctacctggcacggcgatgacttcaaggacaaactgaacttgaaggaatctgatgactctgtgaacactgaggacagaatcctcaagccctgctccacaccgagtgataagctcgtcattgacaaactcgtggttaacttcgggaacgtcctgcaggagatttttaccgacgaagccaggaccggccaggagaataacctgggaggtgagaagaacgggtatgtgacatgcccgttcagacccgattgtccgctggggaagagttttgaagagcttcctgtctctccggaaatcccaccccgcaagagccagtatctccgttcccgcatgccagagggcacccgccctgaggcaaaggagcagctgctcttctctggccagagtctggtgcccgaccacctgtgtgaagagggagcccccaatccatacttgaagaactctgtgacagccagagagttcctggttagcgaaaagctgcctgagcacaccaagggtgaggta

SEQ ID NO: 93 - DNA, 1116 bp, Homo sapiens
  source 1..1116: mol_type = genomic DNA
atggccgcgccagtcccatgggcttgttgcgctgtactggcggccgctgcagccgttgtgtacgctcagcgccactcaccccaggaggctccccacgtacaatatgaacgcctcggttcagacgtcaccttgccgtgtggcacagcaaactgggatgcggccgtgacttggcgcgtgaacggtaccgatctggcccccgacttgctgaacggatctcagctcgtgctgcatggcttggagctggggcatagcggcctgtacgcttgctttcatagagatagctggcacctgcgccaccaggtgctcctgcacgtgggactgccaccccgcgaacctgtgctgagctgtcgcagcaacacttaccccaagggattctactgcagctggcatctgcccacacccacctacatccccaacacattcaatgtgacggtcctccacggctcaaagatcatggtgtgtgaaaaggaccctgccctgaagaaccggtgtcacatccgttacatgcatttgttttctaccattaagtacaaggtttctatcagcgtgtccaacgcattgggtcacaacgcaacagctattactttcgacgagttcacaatcgtgaaaccggatcctccggagaacgtcgttgcccggcccgtccccagtaacccgaggcgcctcgaggtgacctggcagacaccatccacctggcccgatcccgagtctttccctttgaagttctttctccgctatagacctttgatcctggatcagtggcagcacgtcgagctgagtgacggtacagctcacactattacagatgcatacgccggcaaggagtacattatccaggtagcggctaaagacaacgaaatcggaacttggtccgactggtcagtcgcagcccacgccaccccgtggaccgaggaacctaggcacctcactaccgaggcccaagccgctgagacaactacgtcaaccacttcttccctggcgccccctccaaccacaaagatctgtgaccccggggagttgggcagtggcggtggccccagcgccccattcctggtttccgtgccgattactctcgcattggctgccgctgcggccaccgcttctagcctgctgatc

SEQ ID NO: 94 - DNA, 1908 bp, Homo sapiens
  source 1..1908: mol_type = genomic DNA
atgcggggcgggcgcggcgctccgttttggctgtggcccctgcccaagcttgctttgctgccgttgctgtgggtactgttccagcggacccgcccgcaggggtcagcaggccctctccagtgctacggcgtgggcccattgggcgacctcaactgctcttgggagcccctcggggacctgggcgccccgagtgagcttcatttgcagagccaaaagtaccgtagcaacaaaactcagacagtcgctgtcgccgcgggcaggagctgggtagcgatccccagggagcagctgacgatgtccgataagcttctggtgtggggcacaaaggcagggcagccgctgtggcctccagtctttgtcaacctcgagactcagatgaaacccaacgctccacgtctcggtcccgacgtcgacttttctgaagacgatcccctggaggccacggttcactgggctcccccaacatggccaagtcataaggttctgatctgccagttccactataggcgctgtcaggaggccgcttggaccctgctcgagcctgagctcaagactatcccactgaccccggtggagatccaggacctggagctggcaaccggatataaggtatacggccgctgccgcatggagaaggaagaggatctgtggggcgagtggagccccatcctgtctttccaaactcctccaagtgctcccaaggacgtctgggtgtccggcaacctgtgtggaacacctggtggagaggaacctctcttgctctggaaagctcccggaccttgtgtccaggtgagttacaaggtctggttttgggtcggaggtcgcgagctgtcccctgagggcatcacttgctgttgcagcttgattccatctggcgctgagtgggcgcgtgtgtcagctgtgaacgcgacaagttgggagcccctgacaaatttgtccttggtctgcctggactccgcgtccgctccccgcagcgtggcagtctcctctatcgcggggtccaccgagttgctggtgacctggcagccgggccctggcgagcctcttgagcatgttgtggattgggcgcgtgatggagacccgctggagaagctcaactgggtccgcctgcctcccggtaacctgtccgccctgctcccaggcaattttaccgttggcgtgccatatcggattacagtgaccgccgtgagtgctagcgggctggcgtccgcgagctccgtatggggttttagggaagagttggctcccctggtcggccctactctgtggaggctgcaggacgcccctcccggcacacccgccatcgcctggggagaggtgcctaggcatcagctgagaggccacctgactcactatacactctgcgctcagtcagggacatccccctctgtatgtatgaatgtttctggcaatacccaatccgtcacactcccagacctgccttggggcccttgcgagctttgggtgacagcatccactattgctggccagggcccacctggccctattctgcgtctgcacctgccagacaacactctgcgctggaaggtcctgccaggcattctgttcctctgggggctgttcctgttgggctgtggcctcagcctggccacatcaggcaggtgctatcacctgagacacaaggttctgccacgctgggtatgggagaaggtgcctgatccagctaacagctctagcggccagccgcatatggagcaggtgcctgaggcacagccgctgggcgaccttcccatcctggaggtcgaagagatggaaccccctccagttatggagtcttcccagccggcccaggctaccgccccacttgattccggctacgagaagcacttcctgccaaccccggaagagctcggcctgcttggtccccctcgcccgcaggtgctggct

SEQ ID NO: 95 - DNA, 1524 bp, Homo sapiens
  source 1..1524: mol_type = genomic DNA
atggaccacctgggcgcgtctttgtggccacaggtgggctccttgtgtttgctgttggctggagcagcctgggctccccctcccaacttgcccgaccccaagtttgagagcaaggctgcgctcctggctgccaggggtcctgaagagcttctctgtttcactgaaagattggaggacctggtgtgtttttgggaggaagctgcctctgcaggcgtcggcccgggaaattactccttttcctatcagctcgaggacgagccgtggaagctgtgtcggcttcaccaggccccaaccgctcggggtgctgtccgcttctggtgttctctccccaccgcagacactagctccttcgttccgctggagctgagagtcactgcggccagcggcgcccctagataccatcgcgtgattcacatcaatgaggtggttcttctcgacgcccccgtgggccttgtcgccagactcgctgatgagagcgggcacgttgtgctcaggtggctgccaccgcctgagacccccatgacctcccacatccgctatgaagtggacgtttcagccggcaatggtgccgggtcagtccagagggtggagatcttggaaggccgtaccgaatgcgtgctgagtaatttgcgcgggcggacccgctacaccttcgctgtgcgtgctcgtatggccgagcccagcttcggcggattctggtccgcctggtctgagcctgtgagtcttctgacgccatctgacctggatcctttgattctgaccctgagcttgatcctcgtcgtgattctggtcctgctcacggtcctggctctgctttcccatcgtagggcactgaagcagaagatctggcctggcatcccatctccggaatccgagttcgaaggtctgtttaccacacacaaaggtaactttcagttgtggctctaccagaacgatggttgcctgtggtggagtccctgtacgcctttcaccgaggaccctccggcctccttggaagtcctgagtgaaaggtgttggggcactatgcaggcggtggagccaggcaccgacgatgagggcccccttctggagcccgtgggctccgagcatgctcaggacacctacctcgtcctggataagtggctcctgcccaggaaccctccctccgaggacctgcctggccccggaggcagcgtggacatcgttgcaatggacgagggcagtgaggcttcctcttgttcaagcgccctggcctcaaaacctagcccggaaggtgccagcgccgctagcttcgagtatacaatcctcgacccttcctcacagctcctgagaccatggaccctctgtccggagttgccaccgacaccaccccatttgaagtacctgtacctggttgtgtctgacagcggcatctccactgactactcttccggagattcccagggcgcccagggcggactttccgatgggccttattccaacccttacgaaaactctcttatcccagctgcggagcctctgcccccatcttatgttgcctgctcc

SEQ ID NO: 96 - DNA, 1914 bp, Homo sapiens
  source 1..1914: mol_type = genomic DNA
atggacctgtggcaactcctgttgacgctggctctggctggttcctctgacgcgttttccggaagcgaagctacagcagccatcctgagtagagccccctggagcctgcagtctgtcaacccaggactgaagactaattctagcaaggagcctaagttcactaagtgccgtagcccggaacgtgagacgttctcctgtcactggacagatgaggttcatcacggcactaagaacttgggtcccatccaactgttttatacgcgtagaaacacacaagagtggacgcaggaatggaaggaatgtcctgactacgtgagcgccggtgaaaacagctgttatttcaactccagcttcacgtctatttggattccatactgcatcaaactgaccagcaacggtggcaccgtggatgagaagtgcttctcagtcgatgaaatcgtgcagccagaccctccaatcgccctgaactggaccctgctcaacgtcagtctcaccggcatccacgctgatatccaggtgaggtgggaggcccctagaaatgcggatatccaaaaggggtggatggtgctggagtatgagctgcagtataaagaagtgaatgagaccaaatggaagatgatggacccgatccttaccacttccgtgcccgtgtacagcctgaaggttgacaaggaatacgaagtcagagtgcgtagcaaacagaggaactccggtaactatggtgagttctctgaagtgttgtatgtgaccctcccccagatgagtcagttcacctgcgaagaggatttctacttcccgtggctcctgatcattatctttggcatctttggtttgaccgtgatgctgtttgtctttctcttcagcaagcaacagcgtatcaaaatgctgatcctgccgcctgtgccagtgccaaagatcaaaggcatcgacccagacctgcttaaggagggcaagctggaggaagtgaacactatcctggccatccacgattcctacaagccggagttccatagcgacgattcttgggtggagttcatcgagttggacattgacgagcctgacgagaaaactgaggaatccgacaccgacagactgctcagttccgatcatgagaagtcccatagcaacctcggtgtaaaagatggcgatagtgggcgcaccagctgttgcgagcccgacatcctggaaactgactttaatgccaacgatattcatgaaggcacttctgaggttgctcagccacagcgcctgaagggcgaggctgatctgttgtgcttggaccagaagaatcaaaataacagcccttaccatgatgcctgtcccgccactcagcaaccttccgtgatccaggcagagaagaacaagcctcagccactgcccacagagggcgccgagagcacacaccaggcagcccacatccagctgtccaacccatcatccctttctaacatcgacttctatgcccaggtgtctgacattacgccggccggctctgtggtcctgtctcccggccagaaaaacaaggccggcatgtctcagtgtgacatgcatcctgagatggtgagtctttgccaggagaactttctgatggataacgcatatttttgcgaggcagacgctaaaaagtgtatccccgtggctccgcacatcaaagttgagagccatatccagccttctctgaaccaggaggacatctacatcaccactgagtccctgaccactgccgcaggccggccaggcacaggcgagcacgttcccggtagcgagatgcctgtgcccgactatacttctattcatatcgttcagagcccacagggactgattctgaacgcgacagctctgcccctccccgacaaagaattcctgtccagctgtgggtatgtgagcaccgaccagctgaacaagatcatgccc

SEQ ID NO: 97 - DNA, 1866 bp, Homo sapiens
  source 1..1866: mol_type = genomic DNA
atgaaagaaaatgtggcaagcgctacagtgttcacgcttctgctttttcttaacacctgtttgctgaacggacagctgcctcccgggaagccggagattttcaagtgtcgctcccccaataaggagactttcacttgttggtggcgccccggcactgacggtggcttgcccaccaattactccctgacttatcaccgtgagggagaaaccctgatgcacgagtgccctgattatattacaggtggccccaacagctgtcacttcggcaagcagtataccagcatgtggcgcacatatattatgatggttaatgcaacaaaccagatgggcagctctttctccgatgagctgtacgtggacgttacttacatcgtgcagcccgatccacccctcgagctggctgtggaggtgaagcagccggaagatcgcaagccttacctgtggatcaagtggagccctcccactctgatcgacttgaagactggttggtttacattgctgtacgaaatccgcctgaaaccggaaaaggcagctgagtgggaaatccactttgcagggcagcaaaccgagtttaagattctgtccctgcacccggggcagaagtacctggttcaggtgcgttgtaaacctgaccatgggtattggtcagcttggagccctgctaccttcatccagatcccctctgacttcaccatgaacgatactaccgtgtggatcagcgttgctgtgctcagtgccgtgatctgcctgattatcgtctgggccgtggccctcaagggctatagcatggtgacctgtatcttccctccagtgcccggaccgaagatcaagggattcgacgcacaccttctggagaagggaaaatctgaagagcttctctctgccctgggctgccaggacttcccgcccaccagtgactacgaagatcttctggtagaatatctggaagtggatgactccgaggatcagcacctgatgagtgtgcattccaaggaacacccctcccagggcatgaaacctacgtacctggacccagatactgattctggacggggatcctgcgattcaccctccctgctcagcgagaagtgtgaagagcctcaggccaacccctccaccttttatgaccccgaggtgatcgagaaacccgagaatcctgagactacccacacatgggacccgcagtgcatcagcatggagggaaagatcccctacttccatgcagggggctccaaatgctccacctggccgcttcctcagccttcccaacacaaccctaggtcttcctaccacaacatcaccgacgtgtgcgaactggctgtgggccctgccggggccccggcaacactgctcaacgaagccggtaaggacgccttgaagtccagtcagaccatcaagtctcgtgaagagggcaaggctacccaacagagagaagttgaaagcttccattccgaaactgaccaggatacaccgtggctgctcccgcaggagaagacccccttcggatctgccaagcctctggactatgtcgaaatccacaaggtgaacaaagatggtgcactgtccctcttgcccaagcaaagagaaaattccggcaaacccaaaaagccagggacacccgagaataacaaggagtacgcaaaagtgagcggtgtgatggacaataacatcctggtcctcgttccggacccgcacgcgaaaaacgtggcttgtttcgaggaatctgccaaggaagctcccccgtccctggagcagaaccaggctgagaaggccctggcgaacttcacagccacctctagcaagtgtaggctgcaactgggcggactcgactaccttgatccagcctgttttacccacagcttccac

SEQ ID NO: 98 - DNA, 1545 bp, Homo sapiens
  source 1..1545: mol_type = genomic DNA
atgcttctgagccagaacgcattcatcttccgctcccttaacctggttctgatggtgtatatcagtctggtgttcggcatttcttatgacagccccgactacaccgacgagagttgtacgttcaagatcagcctgcggaacttccgcagcattctgagctgggaactgaagaatcatagcatcgtgcccactcattacactttgctgtataccatcatgagcaaacccgaagacctgaaggtggtaaagaactgtgccaacacaacgcgttccttttgcgatttgaccgacgagtggcgcagcacacatgaagcctacgtgaccgttctggagggtttcagcggcaacacgacactgtttagctgtagccacaacttctggctggctattgacatgagcttcgaaccacctgagttcgaaatcgtcggcttcactaaccacatcaacgtgatggtcaagtttccgtccatcgtagaagaggaactgcagtttgacctgtccttggtgatcgaggaacagagtgagggcatcgttaaaaagcacaagcccgagattaaggggaacatgagtgggaactttacgtatattatcgacaaattgattcctaatactaactactgtgtgagcgtctatctcgagcacagcgacgaacaggcagtgatcaagtcccctctgaaatgcaccctcctgccccctggccaagagtccgagtctgccgaatccgcgaagatcgggggtattatcaccgtgtttctgatcgctctcgttcttacatctacaattgtgactcttaagtggatcggctacatctgtctgcgcaacagtctgcctaaggtcctgaacttccataacttcttggcctggcccttccccaacttgcctcccttggaagctatggacatggtagaagtgatctacatcaacaggaaaaagaaagtgtgggactacaactacgacgatgagtctgacagcgacaccgaggccgctccccgcacctctggtggcgggtatactatgcacggtcttactgttcgcccacttggccaggccagcgctactagcaccgaatcccagctgatcgatccagagagcgaagaggaaccggacctgcctgaagtggatgtggaactcccaaccatgcccaaggacagcccccaacagctggagttgctgtcaggaccctgtgagagacgcaagtcacccctgcaggacccatttcccgaggaagactactcaagcaccgagggcagtggcggtcgcatcacgttcaacgtcgacctcaatagtgtcttccttcgtgtgctcgacgatgaggattccgacgatctcgaagctcctctgatgctctcctctcaccttgaagagatggtagaccccgaagatcctgacaacgtgcagtccaaccatctgttggccagcggagaaggcacccagcccacttttccaagcccctctagcgagggtctgtggtccgaggatgctccgagcgaccagagcgacaccagcgagtctgacgtggacctgggggatggttatatcatgagg

SEQ ID NO: 99 - DNA, 1467 bp, Homo sapiens
  source 1..1467: mol_type = genomic DNA
atggctctgctcttcctcctgcccctcgtgatgcagggcgtttctagggccgagatgggaacagccgatctgggtcccagttctgtccctaccccgaccaatgtgactatcgagtcctacaacatgaatccaatcgtctactgggaatatcagatcatgccacaggtacctgtgtttacagtggaagtgaagaattacggcgttaagaactcagaatggattgacgcgtgcatcaacatcagccaccattactgtaacattagcgaccacgtcggcgatcctagcaactcactgtgggtgagggtgaaggctcgtgttggtcaaaaggagtcagcctatgcgaagtccgaagagttcgctgtgtgtagagacggcaaaatcggcccgccaaagttggacattcgcaaggaagagaaacagatcatgattgacatcttccacccttctgtgttcgttaatggtgatgaacaggaggttgactacgacccagaaacgacatgctacatccgcgtgtataacgtgtacgttcgcatgaacggctctgagatccagtataagatcctgacccagaaggaggacgattgtgacgagatccagtgccaactggcaatcccggtgagctccctgaactcacagtattgtgtgtccgcagagggcgtgctgcacgtctggggcgtgacaaccgaaaagtccaaagaggtctgtatcaccatcttcaactcatccatcaaaggctccctgtggatccctgttgtggcagctttgctcctgttccttgtgttgagcctggtcttcatctgcttctatattaaaaagatcaaccctctgaaggagaaaagcattatcctgcctaagtctctgatttctgtggtacgcagcgcaactctggaaacaaaacccgaatccaagtatgtgagcctgattactagctatcaaccgttctctctggaaaaagaagtggtatgcgaggaacctctgtctcctgcaaccgtgcccggtatgcacacagaggataaccctggtaaggtggaacacaccgaagagctgtcttccatcaccgaggtggtcaccacagaagagaacatccccgacgtagtgccgggcagccacttgacccctattgagcgcgagtcctcaagccccctgtcctctaaccagagcgagcccgggagcatcgccctgaactcctaccatagccggaactgctccgagtccgatcactctcgcaacggctttgataccgattcatcctgcctggagagccatagctccctgtctgattcagagtttcccccgaataacaaaggtgagattaagactgagggccaggagctgatcactgtgatcaaagcgcctacgagtttcggctacgacaagccacacgttttggtggatttgctggtggatgactcaggcaaagagagcctgattggctacaggcctactgaagactctaaggagttcagc

SEQ ID NO: 100 - DNA, 1560 bp, Homo sapiens
  source 1..1560: mol_type = genomic DNA
atggctggtcctgaacgctggggccccctgctcctgtgcttgctgcaggcagcgcctggacgcccccgtctggctccgccccagaatgtcacactcctgagccagaacttctccgtgtacctgacttggttgcccgggctcggtaacccacaggacgtgacatactttgttgcctaccaatcttccccaacccggaggcggtggcgtgaggttgaagagtgtgctggcacaaaagagctgctctgctctatgatgtgcctgaaaaagcaggacctgtataacaagttcaaaggccgcgtgaggacggtgagtccgagctccaagtctccatgggtcgaatcagagtacctggactatctgttcgaagtcgagcccgcaccgcccgtgctggttctgacccagaccgaagagatcctgtccgctaacgctacttaccagctgcctccctgtatgccacctctcgatctgaaatacgaggtcgccttctggaaggaaggcgccggtaataagacgttgttccccgtaactccccacggccagcccgttcagatcaccctgcaacctgcggccagcgagcatcactgcctcagcgcccgcactatctacacctttagcgtccccaagtattccaagttttccaagccaacttgctttcttctggaggttcccgaggcaaattgggcattcctggtgctgccttccctgctcatcctgctcctggtgatcgctgcgggcggagtgatctggaagacactgatgggcaatccctggttccaacgcgcaaaaatgccgcgcgccctggacttttcaggccatacgcatcctgtcgccacgttccagcctagccgccccgagagtgtgaacgatctttttctgtgcccccaaaaagagttgacccgcggtgtgcgtcccacacctcgggttagagccccagcaactcaacagacacgctggaaaaaggatctggcggaggacgaagaggaagaggatgaagaggacaccgaagacggcgttagtttccagccctatatcgaaccccctagcttcctcggccaagagcaccaggcccccggacacagtgaagccggcggagtggactccggacgtcccagagcaccactggtgccctctgaaggctccagcgcttgggattccagcgatcgttcctgggccagtaccgtggactcttcctgggatcgcgccggatcctcaggttacttggccgaaaagggccctggccagggccccggcggagacggccaccaagagtccctgcccccacctgagttcagtaaggactctggttttttggaagagctgcccgaagacaatctctcctcttgggccacctggggcaccctgcctccggagccaaacctcgtgccgggtggaccccctgtgagcctgcagacccttactttttgctgggagagctccccagaggaagaggaagaggcacgcgagtccgagattgaagatagcgacgcgggcagctggggtgctgaatccactcagcgtacagaggacaggggtcgcaccctgggccactacatggctcgc

SEQ ID NO: 101 - DNA, 1659 bp, Homo sapiens
  source 1..1659: mol_type = genomic DNA
atgcgcgctcccggccgtcctgccctgcgcccgctgcccctgcccccactgcttctgcttctcctggctgccccttggggccgtgcggttccttgcgtgagcgggggactgcctaagcccgctaacatcacattcctctccatcaatatgaagaacgtcctgcagtggacacccccggagggactgcagggcgtcaaagtgacatacaccgtccagtacttcatctacgggcaaaaaaagtggctgaacaaatccgaatgtcggaacattaaccgcacctactgtgatctgtcagcggagacctcagactacgagcaccaatattacgccaaggtgaaggccatctggggtactaaatgctcaaagtgggccgagtctggtcgtttctatccttttcttgagacccagatcggccctccagaggtggctctgaccacggacgaaaaaagtattagtgtggtcctgaccgcccctgaaaaatggaagcgcaaccctgaggatctgccggtgagcatgcaacagatttactccaatctcaagtataacgtgtcagtgctgaacacgaaatccaaccgcacttggtcacagtgtgtgaccaatcacactctggtgctgacctggttggagcccaacaccctctactgcgtacacgtagagtccttcgtgcccggacctccccggcgtgctcaaccctccgaaaagcagtgcgccagaacactgaaagaccagagctccgaattcaaggctaagattatcttctggtacgtgctccctgtgtctatcacagtgttcctgttctctgtgatgggctattctatctatcgctacatccatgtcggtaaggagaagcaccctgcaaaccttatcttgatctacggtaacgaattcgacaagcgctttttcgtccctgcagagaagatcgtgattaacttcatcactctgaacatctcagatgacagcaagatttctcaccaggacatgagccttctgggcaagtccagtgatgtgagttccttgaacgacccccagccttccggcaacctgaggccccctcaggaggaagaggaagtcaagcacttgggctacgcatcccacctcatggagatcttttgtgactccgaagagaacactgagggtacatccctgacccaacaggaatccctgtcacgcaccattcctcccgacaaaacagtgatcgagtacgagtatgacgttagaactaccgatatctgtgccggtcccgaggaacaggagctcagcctgcaagaggaagttagcacccagggaacactgctcgagtcccaggctgccctcgctgtgctggggccccagacccttcagtacagctatacccctcagctgcaagacttggatccgctggcccaggagcatactgactccgaggaaggacccgaggaagagcccagtacaaccctggtggattgggacccgcagaccggtcgtctgtgcatcccctccctgagctcattcgaccaggactctgaaggctgcgaaccctccgagggggacggtctgggcgaggaaggtctgttgagccgcctgtatgaagagcctgcccctgatcggccaccgggcgagaatgaaacctacctgatgcagtttatggaagagtggggcctgtacgttcagatggagaac

SEQ ID NO: 102 - DNA, 1722 bp, Homo sapiens
  source 1..1722: mol_type = genomic DNA
atgcgcaccttgctgactatcctgaccgtgggctccctggccgctcacgcccctgaggatccctccgatttgctgcagcatgtcaagttccagagctccaactttgagaacatcctgacctgggactcaggtcccgagggtacccccgacactgtgtactccatcgagtataagacctatggcgagcgcgattgggtggctaagaaaggctgtcagcgtattacccgcaagagttgtaacctgaccgtggagacgggcaacctgaccgaactgtactatgccagggtgacggctgtaagcgctgggggccggtccgctaccaagatgaccgaccggttctcctctctccagcacactaccctcaagccgcccgacgtgacctgtatctccaaggtgcgtagcatccagatgattgtgcatccaacccccacccccattcgggctggagacggccatcgcctgaccctggaggacattttccatgacctgttctaccacctggagctgcaggttaatcgcacctatcagatgcacctgggtggcaagcagagggagtacgaattctttggactgacccccgatactgagtttctgggtaccatcatgatctgcgtaccaacgtgggcaaaggagtccgctccctacatgtgccgcgtcaagacactccctgatagaacctggacatactctttcagcggggctttcctcttcagcatgggtttcctggttgccgtcctgtgctacctgtcttatcgctacgtgaccaagccgccagcgccgccaaactcactgaacgtgcagagggtgctcacctttcagcctctcaggttcatccaggagcatgtgcttatccccgtgttcgacctctcaggtccctcctctctggcccagccggtgcagtattcccagatcagagtgtccggtcctcgggagcctgctggcgcaccccagcgtcattccctgagcgaaatcacctacctgggccagcctgacatcagcatcctccagccgtccaatgtacctcccccacagatccttagccctctgtcctacgcgccaaacgcagcccctgaagttggcccacctagctacgctccccaggtaacgcctgaggcgcagttccccttctatgcgccccaggccatcagtaaggttcagccctccagctacgcacctcaggctacacctgattcctggcctccatcctacggcgtgtgcatggagggcagcggaaaggacagccctactggaactctgtcttcccctaagcacctccgtccaaaaggccagttgcagaaggagcctcccgctggttcttgcatgctcgggggcctctctctgcaggaggtgacgtccctggccatggaagagtcccaagaggcaaaaagcctgcaccagcctctgggcatctgcaccgatcgcaccagcgatcccaatgtcttgcacagcggcgaagaggggaccccgcagtacctgaaagggcaactgcccctgctctccagcgtgcagatcgaaggtcacccgatgagcctgccccttcagccccctagccgcccttgtagccctagtgaccagggaccttccccctggggcctcttggagtctctggtatgtccaaaggatgaagcgaagtcccctgccccagagacgtctgatctggaacagcctacggaactcgacagcctgtttcgcggtctggcgttgactgttcagtgggagagt

SEQ ID NO: 103-106 - skipped (4 entries)

SEQ ID NO: 107 - DNA, 831 bp, Homo sapiens
  source 1..831: mol_type = genomic DNA
atggtccgcctgccactgcagtgcgtgctgtggggctgcttgctgaccgcggtacacccggagcctcccactgcctgccgcgagaagcagtaccttatcaattctcaatgttgctctctgtgccagccaggccaaaaactggtgagcgactgcacagagtttaccgagaccgagtgtcttccttgtggagagagcgagttcctggatacatggaaccgcgagacacactgccatcagcacaagtactgtgaccctaacctgggactgcgggtccaacagaagggcacttcagaaacagataccatctgtacgtgtgaagaggggtggcattgcaccagcgaggcctgcgagtcttgtgtcctgcacagaagctgctctcctggtttcggggtcaaacagatcgctaccggcgtcagcgacacaatctgcgagccctgtcccgtggggtttttctccaacgtgtcttccgccttcgagaagtgtcacccctggaccagctgcgagacaaaagacctggttgtgcaacaggcgggcaccaacaagaccgatgtggtctgcggtcctcaggatcgtcttagagctctggtagtgattcctattatcttcggcatcctgtttgccatcttgctggtactggtgtttattaaaaaggtggcgaagaaacccaccaacaaggccccgcatcctaagcaggaaccccaggaaattaactttcccgatgacctcccagggtctaataccgcggcacccgtccaggagacgctccacgggtgtcagcctgtgacccaggaagacggcaaggaaagccgcatttccgtgcaggagcggcag

SEQ ID NO: 108 - DNA, 765 bp, Homo sapiens
  source 1..765: mol_type = genomic DNA
atgggcaactcttgttacaacatcgtcgcaacactcctgttggtgcttaatttcgaacgcacccgcagcctccaggatccatgttcaaactgccctgccggtactttctgcgataataacagaaaccagatctgtagcccatgtcccccaaactctttctccagtgcgggaggtcaacgcacatgtgacatctgtaggcagtgcaaaggagtgttcaggactagaaaggagtgctcatccacaagcaacgctgaatgcgactgcacgccagggtttcactgtctgggtgccggctgttctatgtgtgaacaggattgtaagcagggacaggagctgactaagaaaggttgtaaggactgttgcttcggcaccttcaacgatcagaagaggggtatctgtcgcccctggaccaactgcagccttgacggcaaatctgtgctggtcaatggcaccaaggaacgtgacgtggtctgcggtccctctcccgccgacctgtccccaggggccagctctgtgacccctccggcgcccgcccgcgaacctggtcattctccccaaattatcagttttttcctggcgctgacgagcactgccttgctgttcctgttgtttttcctgaccctgagattctcagttgtgaagcgtggcaggaaaaagttgctgtatattttcaaacagcctttcatgagaccagtgcaaaccacgcaggaagaggatggctgttcctgccgcttccctgaagaggaagagggcgggtgcgagttg

SEQ ID NO: 109 - DNA, 888 bp, Homo sapiens
  source 1..888: mol_type = genomic DNA
atggcggctgggggccctggagccggttctgcagcccccgtgtcatccactagtagcctgcccctggcggctctgaatatgcgcgtgcggcgtcgcctcagtttgttcctcaatgtgaggactcaggtggcggcagattggacagctcttgccgaagagatggattttgagtacctcgagatccgccagctggaaactcaggccgaccctaccggacgcctgctcgacgcttggcagggtcgccctggtgcctctgtgggccgtcttctggaattgctcaccaagcttggtcgcgacgatgtactcctggagctgggaccttcaattgaagaggattgtcagaagtacatcctgaaacagcaacaggaagaggccgagaagccgctgcaggtcgccgcagttgactcttcagtgccaaggactgccgagctggcaggaatcacgaccctggatgaccccctcggccacatgcctgagcggttcgatgccttcatttgttactgcccttcagatatccagttcgtgcaagagatgatcagacagctggagcagaccaactaccggctgaagttgtgcgtgtccgatcgcgacgtgttgccgggcacctgcgtgtggtcaatcgccagcgagctgattgagaagcgctgtcggcgtatggtcgtggtagtgtccgatgactatttgcagagtaaggagtgcgacttccagaccaaattcgccttgagcctttcccccggtgctcatcagaaacgtctgattccaatcaagtacaaggccatgaaaaaggaattccctagtattcttcgctttattactgtttgtgactacaccaatccatgcaccaagtcctggttctggacgcgcctggccaaagcgctgtcactgcca

SEQ ID NO: 110 - DNA, 831 bp, Homo sapiens
  source 1..831: mol_type = genomic DNA
atgtgtgtgggcgcacgcaggctgggcaggggcccctgcgctgcacttctgctcctggggctgggtttgtccaccgtgaccggactgcactgcgtcggcgacacctatccaagcaacgacaggtgttgccatgagtgtagaccaggcaatggtatggtgagccgctgcagtcgcagccagaataccgtgtgtagaccttgtggtcctggcttttataacgacgtggtcagttccaaaccgtgtaaaccgtgtacgtggtgcaatctgcgtagcggatctgaacgcaagcagctctgcaccgccacgcaggacacggtctgtcgctgccgcgctggcacccagccgttggacagttacaaaccaggtgttgactgtgcaccttgtcccccaggccacttcagcccgggagacaatcaggcctgcaaaccctggaccaactgcaccctggctggtaaacatactctccagcctgcatccaacagctctgatgctatctgcgaggaccgcgatccccctgccacccagccccaggagacccaaggtccacctgcccgccctatcaccgtgcagcccactgaagcctggccaaggacctcacagggtccttccacacggccggtggaggtgcctggaggcagagccgtcgctgcgatcctcggtcttggcctcgtgctgggtctcctgggacctctcgctatcctcctggccctgtatctgcttagaagggatcagcgcctgccccctgatgcccataagccgccagggggtggctcttttcgcaccccaattcaggaggaacaggccgatgcccactctactctggctaagatc

SEQ ID NO: 111 - DNA, 1785 bp, Homo sapiens
  source 1..1785: mol_type = genomic DNA
atgcgcgtgctgctcgccgctcttggcttgctgttcctgggtgctctgcgtgctttcccccaggacagacccttcgaggacacatgccacggcaaccccagccattactatgacaaagcagtgcgtcgctgctgttaccgttgtccgatggggctgttccctacgcaacagtgcccccagagacccaccgattgtcgtaagcagtgcgaacctgattattacctggacgaggccgataggtgcacagcctgcgtgacatgctcccgtgacgatctggtagaaaaaactccctgtgcttggaacagctccagggtgtgcgagtgtcgccctggcatgttctgttctaccagcgcagtgaacagttgtgcgaggtgtttctttcacagcgtctgcccggccggcatgatcgtgaagttcccaggcaccgcccaaaagaacaccgtctgtgaacctgcctctcctggtgtaagcccagcttgcgcctcacccgagaactgcaaagagccctcaagcggcaccatccctcaggccaagcccacacccgtatcccctgcgacgtcctctgcctccaccatgcctgtcagaggaggcacccgtttggctcaggaggcggccagcaagctgacccgcgctcctgacagccccagtagcgtgggacgcccatctagcgatccgggcttgtctccgacgcaaccttgccctgagggctccggcgactgtcgtaagcagtgtgaaccggattattacctggacgaggctggccgctgcaccgcatgtgtctcctgtagccgcgatgacctggtagagaagacaccatgcgcctggaactcttcccgtacctgcgagtgcagaccaggaatgatttgtgccacgagcgcaaccaattcctgcgcgcgctgtgtgccctatcccatctgtgcggctgagaccgtcacgaagcctcaggatatggccgagaaagacaccacgttcgaagcaccccctctgggtacgcagcccgactgtaaccctaccccagaaaatggcgaagcgccggcaagtacctctccaactcaatccctcctggtcgacagccaggcctccaagaccctccctatccccacctccgcccccgtcgcactgagctccaccggtaagcccgtcctggatgccggtccagttttgttctgggtgatcctggtgctggtcgttgtggtcggatctagtgcctttttgctgtgtcatcgtcgcgcttgtcgcaaacgtatccgtcagaagctgcacctctgctatcctgtgcagactagtcagccgaagctggaactggtggacagcagacccaggcgttcaagcacccagctgcgctctggcgcatccgtgactgaaccagttgcagaggaacgtggtctcatgtcccagcctctgatggaaacctgccactctgtgggcgccgcttatttggaatctctccctctccaggacgcctcaccggctggcggaccttctagcccccgcgacctgccggagccaagggttagcacggagcacaccaataacaagatcgaaaagatctacatcatgaaggcagacacagttattgtgggcacagttaaggcggaactgcctgaaggccgcgggctggcgggaccggctgagcccgagttggaagaggaactggaggccgatcacacaccccactaccccgagcaggaaaccgagcctcccctcggtagctgcagtgacgtaatgctgtctgtggaagaggaaggcaaggaggaccctctgcctactgcagcctctggaaag

SEQ ID NO: 112 - DNA, 1365 bp, Homo sapiens
  source 1..1365: mol_type = genomic DNA
atgggtctgtctacagtgcccgacctgctcctgcccctggtgcttctcgaactcctggtgggaatttatcccagcggcgtgatcggtctcgtgccccacttgggcgatagggagaaacgcgatagtgtctgcccccaggggaagtacatccacccccaaaataactccatctgctgtaccaagtgtcataagggtacctacctgtacaacgactgtcctggtccaggacaggacacggattgccgcgaatgtgagagcggttccttcacggctagcgagaaccatctcaggcattgtctctcctgcagcaagtgtagaaaggaaatgggtcaggtggagatttcttcctgcactgtggaccgtgacacggtgtgcggctgtcgcaaaaaccagtacaggcattattggtcagagaacctgttccagtgcttcaactgtagcctctgtctgaacggcacggtgcacctgtcatgtcaggagaagcagaacactgtgtgtacatgccatgccggctttttcctgagagagaatgagtgtgtgagttgtagcaattgcaaaaagtccctggaatgcacaaagctgtgtttgccacagatcgagaatgtgaagggtaccgaggattctggaaccacagtgctgcttccactcgtaatcttctttggcttgtgtttgctgtctctgctcttcatcggtctcatgtatcgctatcagcgttggaagagcaagctgtattccatcgtgtgcggtaagagcactcccgagaaggaaggagaactggagggcacaaccacaaagcccttggcccctaacccgagcttctctccaactccaggattcaccccaacactgggcttctctcctgtgccgtccagcaccttcacttcttccagcacatacacccccggagattgtccaaatttcgccgctcctcgtcgcgaagtggcgccgccttatcagggtgccgacccgattctggcaaccgcactggcctcagacccaatccctaaccctctccagaagtgggaggactccgctcataaacctcagagtctcgacaccgatgacccggccaccctgtacgccgtcgtggagaacgttcccccactgagatggaaggagttcgtgcgtcgcctcggcttgtctgaccacgaaattgacaggttggagctgcaaaatggtaggtgcctccgcgaggcacaatactccatgctcgcgacatggaggcgccggaccccgaggcgcgaagccactctggagctcctgggacgtgtactgcgtgacatggatctcctgggttgcctggaagacatcgaagaggccctgtgcggccctgcggctctgcctcccgcaccctctttgctgagg

SEQ ID NO: 113 - DNA, 1383 bp, Homo sapiens
  source 1..1383: mol_type = genomic DNA
atggcccctgtagcagtgtgggcggccctggccgtggggctggagctctgggccgcggcccatgccctgcccgctcaagtggcattcactccctacgcgcccgagcccggctccacctgcaggctgcgtgagtattacgaccagaccgctcagatgtgttgcagtaagtgtagccctggccagcacgccaaggtgttctgtaccaagacgtctgataccgtgtgcgactcttgtgaagattccacctacacccagctgtggaactgggtgcctgagtgcttgtcttgtggctctcgctgcagctcagaccaggtagagacccaggcctgtacccgcgagcagaaccgcatctgtacctgcaggcccggctggtactgcgctctgtccaagcaggaggggtgcagactgtgcgcgcccctccggaagtgccgtcccggctttggagtggcacgtcctggtaccgagacatctgacgtggtttgcaagccatgcgcccctgggaccttctctaacaccacatccagcacggacatttgcagacctcatcagatttgtaacgtcgtggccatccctggcaacgcctccatggatgccgtctgcacctccacctctccgacccgcagtatggcccccggcgcagtgcacctgccgcagcccgtatcaacccgctcccaacatactcagcctacaccagagcctagcaccgccccctccacatccttcttgctgcctatgggcccgagccctcccgctgaaggctccacaggtgacttcgcgctcccggtgggactgatcgtgggcgtcactgccctcggcctgctcatcattggtgttgtgaattgtgtcatcatgacccaggttaagaaaaagcccctgtgtctgcagcgtgaagctaaggtcccgcacctgccggcggacaaggcacgcggcactcaagggcctgagcaacagcacctcctgatcaccgcccccagtagctccagttcttccctcgagagctctgcctctgccctggataggcgtgcgccaacacgcaaccagccgcaggccccaggcgtggaggcctcaggagcaggggaggcacgcgcttccaccggcagctccgactcctctcctggcggtcacggaacccaggtgaatgtcacttgtattgtcaacgtgtgtagctcctctgatcactctagccagtgctcttcccaggcgagtagcactatgggggacactgactccagcccctccgagagtcccaaggacgagcaggtcccgttctccaaggaagagtgtgcgtttcggtcccagctcgaaactccagagacgcttctgggatccacggaagagaagcccctgcccctgggtgtcccagacgcgggcatgaagccatcc

SEQ ID NO: 114 - DNA, 2979 bp, Homo sapiens
  source 1..2979: mol_type = genomic DNA
atgcctgcgctggcgagggatggaggccagttgcccttgctggtggttttcagtgcaatgatcttcggtaccatcacaaaccaagacctccccgtcatcaaatgtgtcctcatcaaccacaaaaacaatgactcatctgtggggaagagctcttcctatccaatggtttctgaaagcccagaggaccttggctgcgcgctccgccctcagtccagtggtacggtctacgaggctgccgctgtggaggttgacgtcagtgcttccatcacccttcaggtcctggttgacgctcctggcaacatctcctgtttgtgggtgttcaagcacagctccctgaactgccagcctcattttgacctccaaaacagaggcgttgtgtccatggtcatcttgaagatgaccgaaacccaggcaggggaatacctcctgttcatccaatccgaagcgaccaactacaccatccttttcaccgtctctatcaggaacaccctcctgtatactctgcgtcgcccttactttcgcaaaatggagaaccaggacgctctggtgtgtatcagcgagtctgttcctgagcctattgtggagtgggttctctgtgacagccagggcgagtcctgtaaggaggaatcccccgctgtggttaaaaaggaagagaaggtactgcacgagctgttcggcaccgacattcgttgctgtgcacggaacgagctgggccgcgaatgcactcggcttttcaccatcgatctcaaccagacgcctcagactaccctgccccaactgttcctcaaagtgggggagcccctgtggattcgttgcaaggccgtccacgtcaaccatggtttcggtctgacctgggaattggagaacaaagctctggaggaaggtaactactttgaaatgtctacctactctaccaaccgcactatgatcaggatcctcttcgccttcgtgagctccgtggcccggaatgacactggctattacacatgcagctcctctaagcacccatcccaaagcgcgctggtgaccatcgtagagaagggctttattaacgctaccaactcttccgaggactacgagatcgaccaatacgaagagttctgtttctccgtgcgcttcaaagcttacccgcagattcgctgtacatggaccttttctagaaagagcttcccgtgcgagcagaagggtttggacaacggctactcaatctccaaattctgtaaccacaagcaccaaccgggcgagtacatctttcatgctgagaatgatgacgcccagtttaccaaaatgttcaccctgaacattcgtcgcaagcctcaggtcctcgcagaggctagtgcctcccaggcgtcctgtttctccgatggttaccctctccctagctggacctggaaaaagtgttctgacaaatcccctaactgcacagaggaaatcactgagggtgtctggaatcgcaaggctaaccgcaaggtgtttggccagtgggtctcttccagtaccctcaacatgagcgaagccatcaagggctttctggtcaagtgttgcgcgtacaattccctgggcaccagctgtgagaccattctgcttaacagccccggaccttttcctttcatccaggataacattagtttttacgctacaattggagtgtgtttgctgtttatcgtggttctgactttgctgatctgtcacaagtacaagaaacaattcagatacgagtcccagctccagatggtgcaggttaccgggtcttcagacaacgagtatttctacgtggattttcgcgaatacgagtatgatctgaagtgggagttccctagagagaacctggaatttggcaaagtgctgggatccggtgcctttggcaaggtgatgaacgcaacagcctacggtatcagcaagactggcgtgtctatccaggtggcagtgaaaatgcttaaggaaaaggctgattcctctgagcgcgaggccttgatgtccgagttgaagatgatgacacagctgggctctcacgagaacatcgtgaacctcctgggggcctgcaccctgtccgggcccatctatctcatcttcgagtactgttgctatggcgacctcctgaactatctcaggagtaagagggagaaattccacaggacctggaccgagatcttcaaagaacataacttctccttttaccccaccttccagtctcaccctaactcttccatgccaggaagccgtgaagtgcagattcaccccgactccgaccagatctccggtttgcacggcaactccttccacagcgaggatgaaatcgagtacgagaaccagaagcgtctggaggaagaggaagacctgaacgtgctgacatttgaggacttgctgtgcttcgcctatcaagtggcaaagggcatggaatttctggaattcaagtcttgtgtccaccgcgaccttgctgcgcgcaacgtactggtcacccatggtaaagttgtgaagatctgtgatttcggcctggcccgcgacatcatgtctgattccaactatgtcgtgaggggcaatgcccgtttgcctgttaagtggatggctccggagtctctgtttgagggcatctacaccatcaagagtgacgtatggagctatggtattctgctttgggagatcttcagtctgggcgtcaacccttatccaggtattcccgtggacgcgaacttttacaagctgattcagaacggcttcaaaatggaccagccattctacgcgaccgaggaaatctacatcattatgcagtcttgttgggccttcgattcccgcaagcggccatcttttccaaatctcacctcctttctcggttgtcagctggcggacgctgaggaagcaatgtaccagaacgttgacggtcgcgtgtccgaatgtccgcacacctaccaaaaccgccgtccattttcccgcgagatggacctggggttgctgtccccgcaggcacaagtggaagacagc

SEQ ID NO: 115 - DNA, 702 bp, Homo sapiens
  source 1..702: mol_type = genomic DNA
atgcgcaaaactagactgtggggtctcctgtggatgttgttcgtctccgagctgagggcagccactaagctgactgaggaaaagtatgaactgaaggaaggccaaactttggatgtgaagtgcgattacaccctcgagaagtttgcctctagccagaaggcgtggcagatcattcgcgatggtgagatgcctaagactctggcctgtacagagcgccccagcaaaaattcccatccggtgcaggtggggagaattatcctggaggattaccacgatcacggccttctgcgtgttcgcatggtgaacctgcaagttgaggacagcggcttgtaccagtgtgttatctaccagcctccgaaggagccacatatgctgtttgaccgcatccgcctggtggtaactaagggcttctccggcacccctggttcaaatgaaaactccacccagaacgtgtacaagatccctccaacaaccacaaaggccctctgcccactgtacacgagcccgcgcactgtaacccaggctcctccgaagagtactgcagacgtctctacgccagattctgagatcaaccttaccaacgtgacagacatcattcgcgttcctgtgttcaatatcgtcatcttgctggccggtggcttcctgtctaagagcctggtgttcagtgtgctcttcgcagtcacactgcgcagttttgttcct

SEQ ID NO: 116 - DNA, 690 bp, Homo sapiens
  source 1..690: mol_type = genomic DNA
atggagccgttgcgtctgctcatcctcctgtttgtcaccgagctcagcggcgcgcacaacactaccgtgttccagggggtggctggccagagcctccaagtgtcttgtccctacgacagcatgaagcattggggacgcagaaaggcgtggtgccgccagctgggagagaaaggcccctgccagcgcgtggtctccacacacaatctgtggctcctgtctttcctgcgtcgctggaatggtagcaccgccatcaccgatgacactcttggaggcaccttgactattactctgagaaacctgcagccacacgacgctgggttgtaccaatgccagagcctgcatggatctgaggccgacaccctgcgcaaggtactggtagaggtgttggctgatccgcttgaccacagagacgccggcgacctgtggttccccggcgaatctgagagctttgaggacgcccatgttgagcacagtatttcccgcagccttctggaaggcgagatcccgttcccgcccacatccatcctgctcctgttggcctgtatcttcttgattaagatcctggccgctagcgctctttgggccgctgcctggcacggccagaagcctgggacccacccaccctccgaactggactgtgggcatgacccaggttaccaactgcagactttgcccggcctgcgcgacacc

SEQ ID NO: 117 - DNA, 813 bp, Homo sapiens
  source 1..813: mol_type = genomic DNA
aaaaccctgatgggcaacccctggtttcagcgggccaagatgcctagagcactggactttagcggccacacacaccccgtggccacctttcaacctagcagacctgagagcgtgaacgacctgttcctgtgtcctcagaaagaactgaccagaggcgtgcggcccacacctagagtcagagcaccagctacacagcagaccagatggaagaaggacctggccgaggacgaggaagaggaggacgaagaggataccgaggacggcgtgtccttccagccttatatcgagcctcctagcttcctgggccaagagcatcaggcccctggacattctgaagccggcggagttgatagcggcagaccaagagcacctctggtgcctagcgaaggatctagcgcctgggacagcagcgatagaagctgggccagcacagtggatagcagctgggatagagccggcagctctggatatctggccgagaaaggaccaggacaaggccctggcggagatggccaccaagaatctcttccacctcctgagttcagcaaggacagcggctttctcgaggaactgcccgaggacaatctgtccagctgggctacatggggcacactgcctccagagcctaatctggttcctggcggacctcctgtgtctctgcagaccctgaccttttgctgggagagcagccccgaggaagaagaagaggccagagagtccgagatcgaggattccgatgccggatcttggggagccgagagcacacagagaaccgaggatagaggcagaaccctgggccactacatggccaga

SEQ ID NO: 118 - DNA, 753 bp, Homo sapiens
  source 1..753: mol_type = genomic DNA
aagtggatcggctacatctgcctgagaaacagcctgcctaaggtgctgaacttccacaactttctggcctggccttttcctaacctgcctcctctggaagccatggacatggtggaagtgatctacatcaaccggaagaagaaagtctgggactacaactacgacgacgagagcgacagcgacacagaggccgctcctagaacatctggcggcggatatacaatgcacggcctgaccgttagacctctcggacaggcctctgccacaagcacagagagccagctgatcgaccctgagagcgaggaagaacctgacctgcctgaggtggacgtggaactgcctaccatgcctaaggacagccctcagcagctggaactgctctctggcccttgcgagagaagaaagagccctctgcaggacccatttcctgaagaggactacagctccaccgaaggctctggcggcagaatcaccttcaacgtggacctgaacagcgtgttcctgagagtgctggacgacgaggatagcgacgacctggaagctcctctgatgctgagcagccacctggaagagatggtggaccccgaggatcccgacaacgtgcagagcaatcatctgctggctagcggcgagggcacccagcctacatttccatctccaagcagcgaaggcctttggagcgaggatgcccctagcgatcagagcgataccagcgagtccgatgtggacctcggcgacggctatatcatgcgg

SEQ ID NO: 119 - DNA, 669 bp, Homo sapiens
  source 1..669: mol_type = genomic DNA
tgcttctacatcaagaagatcaacccgctgaaagagaagtccatcatcctgcctaagagcctgatcagcgtcgtgcgctctgccacactggaaacaaagcccgagagcaaatacgtgtccctgatcaccagctaccagcctttcagcctggaaaaagaggtcgtctgcgaggaacctctgagccctgctacagtgcctggcatgcacaccgaggacaatcccggaaaggtggaacacacagaggaactgagcagcatcaccgaggtggtcaccaccgaagagaacatccccgatgtggtgccaggcagccacctgacacctatcgagagagagtctagcagccctctgtccagcaatcagagcgagcctggatctatcgccctgaacagctaccacagccggaactgcagcgagagcgaccacagcagaaacggcttcgacaccgacagcagctgcctggaatctcacagcagcctgagcgacagcgagttccctccaaacaacaagggcgagatcaagaccgagggccaagagctgatcaccgtgattaaggcccctaccagcttcggctacgacaagcctcatgtgctggtcgatctgctggtggacgacagcggcaaagagtctctgatcggctacagacccaccgaggatagcaaagagttcagc

SEQ ID NO: 120 - DNA, 36 bp, Homo sapiens
  source 1..36: mol_type = genomic DNA
cctaccagcttcggctacgacaaaccccatgtgctg

SEQ ID NO: 121 - DNA, 966 bp, Homo sapiens
  source 1..966: mol_type = genomic DNA
cagctgtatgtgcggcggagaaagaaactgcccagcgtcctgctgttcaagaagccctctccattcatcttcatcagccagcggccttctccagagacacaggacaccatccatcctctggacgaagaggccttcctgaaggtgtcccctgagctgaagaacctggatctgcacggcagcaccgatagcggctttggcagcacaaagcctagcctgcagaccgaggaaccccagttcctgctgcctgatcctcatcctcaggccgatagaaccctgggcaacagagaacctcctgtgctgggcgatagctgtagcagcggcagcagcaatagcaccgactccggcatctgtctgcaagagccttctctgagcccaagcacaggccctacatgggagcagcaagtgggcagcaattccagaggccaggatgacagcggaatcgacctggtgcagaactctgaaggcagagccggcgatacacaaggcggatctgctctgggacaccactctccacctgagcctgaagttcccggcgaagaggatcctgccgctgtggcatttcagggctacctgagacagaccagatgcgccgaggaaaaggccaccaagaccggctgtctggaagaggaatcccctctgacagatggactgggccccaagttcggcagatgcctggttgatgaagccggactgcatcctcctgctctggccaagggatacctgaagcaggaccctctggaaatgaccctggcttcttctggcgcccctaccggacagtggaatcagcctacagaggaatggtccctgctggccctgagcagctgtagcgatctgggcatcagcgattggagcttcgcccacgatctggccccactgggatgtgttgctgcacctggtggactgctgggctccttcaatagcgacctggtcaccctgccactgatcagcagtctgcagagcagcgag

SEQ ID NO: 122 - DNA, 33 bp, Homo sapiens
  source 1..33: mol_type = genomic DNA
ctggccaagggctacctgaagcaggaccctctg

SEQ ID NO: 123 - DNA, 2670 bp, Homo sapiens
  source 1..2670: mol_type = genomic DNA
atggccctgagaagatccatgggcagacctggacttccacctctgcctttgcctccacctcctagactgggactgctgctggctgctcttgcttctctgctgctgcctgaatctgccgccgctggactgaaactgatgggagcccctgtgaagctgaccgtgtctcagggacagcccgtgaaactgaactgcagcgtggaaggcatggaagaacccgacatccagtgggtcaaagatggcgccgtggtgcagaacctggaccagctgtatatccccgtgtccgagcagcactggatcggctttctgagcctgaagtccgtggaaagaagcgacgccggcagatactggtgccaggttgaagatggcggcgaaaccgagatcagccagcctgtgtggctgacagtggaaggcgtgccattcttcaccgtggaacccaaggatctggccgtgcctcctaacgctccattccagctgtcttgcgaagccgtgggacctcctgagcctgtgacaatagtttggtggcggggcaccacaaagatcggcggacctgctccttctccttccgtgctgaatgtgaccggcgtgacccagagcaccatgtttagctgcgaggcccacaacctgaaaggcctggcctctagcagaaccgccaccgtgcatcttcaagccttgcctgccgctcctttcaacatcaccgtgaccaagctgagcagcagcaatgcctctgtggcttggatgcctggcgctgatggtagagcactgctgcagagctgtaccgtgcaagtgacacaagctccaggcggctgggaagtgctggctgtggttgttcctgtgcctcctttcacctgtctgctgagagatctggtgcctgccaccaactacagcctgagagtcagatgcgccaacgctctgggcccttctccatatgctgattgggtgcccttccagaccaaaggactggcccctgcttctgcccctcagaatctgcacgccatcagaaccgatagcggcctgatcctggaatgggaagaagtgatccccgaggctcctctggaaggacctctgggaccttacaagctgtcctgggtgcaagacaacggcacacaggacgaactgaccgtcgagggcacaagagccaatctgacaggctgggaccctcagaaagacctgatcgtcagagtgtgcgtgtccaacgccgttggctgtggaccttggagtcagcctctggtggtgtcctctcacgatagagctggacagcagggccctcctcacagcagaacatcttgggtgccagtggtgctgggcgtgctgacagctcttgttacagctgctgccctggctctgatcctgctgcggaagcggagaaaagaaaccagattcggacaggccttcgacagcgtgatggccagaggcgaacctgccgtgcactttagagccgccagaagcttcaacagagagcggcccgagagaatcgaggccacactggattctctgggcatcagcgacgagctgaaagaaaagctggaagatgtgctgatcccggaacagcagttcaccctgggcagaatgctcggcaagggcgagtttggatctgtgcgggaagcccagctcaagcaagaggatggcagcttcgtgaaggtggccgtgaagatgctgaaggccgacatcattgccagcagcgatatcgaagagttcctgcgcgaagccgcctgcatgaaggaattcgatcaccctcacgtggccaagctcgtgggagtgtctctgagaagcagagccaaaggcaggctgcccattcctatggtcatcctgcctttcatgaagcacggcgacctgcacgcctttctgctggcctccagaatcggcgagaaccccttcaatctgcctctgcagaccctgatccggttcatggtggatatcgcctgcggcatggaatacctgagcagccggaacttcatccaccgcgatctggctgccagaaactgcatgctggccgaggatatgaccgtgtgcgtggccgattttggcctgagccggaagatctacagcggcgactactacagacagggctgcgcctctaagctgcccgttaagtggctggccctggaaagcctggccgacaatctgtacaccgtgcagtctgacgtgtgggcctttggcgtgaccatgtgggagatcatgaccagaggccagacaccttacgccggcatcgagaacgccgagatctacaactatctgatcggcggcaaccggctgaagcagcctcctgagtgtatggaagatgtctacgacctgatgtaccagtgttggagcgccgatcctaagcagcggcctagctttacctgcctgagaatggaactggaaaacatcctgggccagctgagcgtcctgagcgcttctcaggatcccctgtacatcaacatcgagagagccgaggaacccacagccggcggatctcttgaactgcctggcagggatcagccttattctggtgctggcgacggctctggcatgggagctgttggaggaaccccaagcgactgccggtacattcttacacctggcggactggccgaacagcctggacaggctgaacaccagccagagagccctctgaacgagacacaacgtctgctgctcctccagcagggactgctccctcactcttcttgt

SEQ ID NO: 124 - DNA, 2682 bp, Homo sapiens
  source 1..2682: mol_type = genomic DNA
atggcttggagatgccccagaatgggcagagtgcctctggcttggtgtctggctctttgtggctgggcctgtatggcccctagaggaacacaggccgaggaatccccattcgtgggcaaccccggaaatatcacaggcgccagaggactgaccggcacactgagatgtcaactgcaggttcagggcgagcctcctgaagtgcactggctgagagatggccagatcctggaactggccgacagcacacagacacaggtgccactgggagaagatgagcaggacgactggatcgtggtgtcccagctgagaatcaccagcctgcagctgagcgataccggccagtatcagtgcctggtgtttctgggccaccagaccttcgtgtctcagcctggatatgtgggcctcgagggcctgccttacttcctggaagaacccgaggatagaaccgtggccgccaacacacccttcaacctgtcttgtcaggcccagggacctcctgagcctgttgatctgctgtggctgcaggatgccgtgcctcttgctacagctcctggacacggacctcagagaagcctgcatgtgcctggactgaacaagaccagcagcttcagctgcgaagcccacaatgccaagggcgtgaccacaagcagaaccgccacaatcacagtgctgccccagcagcctagaaacctgcacctggtttccagacagcccaccgagctggaagtggcttggacacctggcctgagcggcatctatcctctgacacactgtacactgcaggccgtgctgagcgacgatggcatgggaattcaagccggcgagcctgatcctcctgaggaaccacttacaagccaggcctctgtgcctcctcatcagctgagactgggatctctgcaccctcacacaccctaccacatcagagtggcctgcacaagctctcagggccctagctcttggacccactggctgcctgtggaaacacctgaaggcgttccactgggccctccagagaatatcagcgccaccagaaatggctcccaggccttcgtgcattggcaagagcctagagcacctctgcagggcacactgctgggatacagactggcctaccagggccaagatacccctgaggtgctgatggacatcggcctgagacaagaagtgacactggaactgcaaggcgacggcagcgtgtccaatctgacagtgtgtgtggccgcctatacagccgctggcgacggaccttggtcactgcctgttcctctggaagcttggaggcctggacaggcacagcctgtgcatcagctggtcaaagagccttccactcctgccttcagctggccttggtggtacgttctgctgggagctgtggtggctgctgcctgcgttctgattctggccctgttcctggtgcaccggcggaagaaagagacaagatacggcgaggtgttcgagcccacagtggaaaggggagagctggtcgtccggtacagagtgcggaagtcctacagcagacggacaaccgaggccacactgaacagcctgggaatcagcgaggaactgaaagaaaagctgcgggacgtgatggtggatcggcacaaagtggccctgggcaagacactcggagagggcgaatttggcgccgtgatggaaggacagctgaaccaggacgacagcatcctgaaggtggccgtgaaaaccatgaagatcgccatctgcaccagaagcgaactcgaggactttctgagcgaggccgtgtgcatgaaggaattcgatcaccccaacgtgatgcggctgatcggagtgtgttttcagggcagcgagcgcgagagctttcctgctcctgtggtcatcctgcctttcatgaagcacggcgacctgcacagctttctgctgtactccagactgggcgaccagcctgtgtacctgcctacacagatgctggtcaagttcatggccgatatcgcctccggcatggaatacctgagcaccaagcggttcatccacagagatctggccgccagaaactgcatgctgaacgagaacatgagcgtgtgcgtggccgatttcggcctgtccaagaagatctacaacggcgactactaccggcagggcagaatcgccaagatgcccgtgaagtggatcgccattgagagcctggccgatagagtgtacaccagcaagagcgacgtgtggtccttcggcgtgacaatgtgggagatcgccaccaggggacagaccccttatcctggcgtggaaaacagcgagatctacgactacctgagacagggcaacagactgaagcagcccgccgattgtctggatggactgtacgccctgatgagccggtgttgggagctgaaccctcaggacagacctagctttaccgagctgagagaggacctggaaaacaccctgaaagccctgcctccagctcaagagcccgacgagatcctgtacgtgaacatggatgaaggcggcggataccctgaaccacctggtgctgctggcggagctgatccacctacacagcccgatcctaaggatagctgctcctgcctgacagccgccgaagttcatccagccggcagatacgtgctgtgccctagcacaacaccctctccagcacagccagccgacagaggatctccagctgctccaggacaagaagatggcgct

SEQ ID NO: 125 - DNA, 2997 bp, Homo sapiens
  source 1..2997: mol_type = genomic DNA
atgggacctgctcctctgcctctgctgctgggactgtttctgcctgctctttggcggagagccatcaccgaggccagagaggaagccaagccttatcctctgttccccggaccttttccaggcagcctgcagaccgatcacacccctttgctgtctctgcctcacgcctctggctatcagcccgctctgatgttcagccccacacagccaggcagacctcacacaggcaatgtggccattcctcaagtgaccagcgtggaaagcaagcccttgcctcctctggccttcaagcacacagtgggccacatcatcctgagcgagcacaagggcgtgaagttcaactgcagcatcagcgtgcccaacatctaccaggacaccaccatcagctggtggaaggacggcaaagaactgctgggagcccaccacgccatcacacagttctaccccgacgatgaagtgaccgccatcattgccagcttcagcatcaccagcgtgcagagaagcgacaacggcagctacatctgcaagatgaagatcaacaacgaggaaatcgtcagcgaccccatctacatcgaggtgcagggcctgcctcacttcaccaagcagcccgagagcatgaacgtgaccagaaacaccgccttcaacctgacctgtcaggccgtgggacctcctgagcctgtgaacatcttctgggtgcagaacagctccagagtgaacgagcagcctgagaagtcccctagcgtgctgacagtgcctggactgacagagatggccgtgttttcttgcgaggcccacaacgacaagggcctgaccgtgtctaagggcgtgcagatcaatatcaaggccattccatctccacctaccgaggtgtccatccggaatagcacagcccactccatcctgatctcttgggtgcccggcttcgatggctacagccccttcagaaactgctccatccaagtgaaagaggccgatcctctgagcaacggctccgtgatgatcttcaacacaagcgccctgccacacctgtaccagatcaaacagctgcaggctctggccaactactccatcggcgtgtcctgcatgaacgagatcggctggagtgccgtgtctccttggatcctggccagcacaactgaaggcgctccatctgtggcccctctgaatgtgaccgtgttcctgaacgagagcagcgacaatgtggacatccggtggatgaagccacctacaaagcagcaggacggcgaactcgtgggctacagaatctctcacgtgtggcagtctgccggcatctccaaagaactcctcgaagaagtgggccagaacggcagcagagccaggatctctgtgcaggtccacaacgccacatgcacagtgcggattgccgctgtgacaagaggcggagtgggcccttttagcgaccccgtgaagatctttatccccgctcacggctgggtcgactacgccccatcttctacaccagctccaggcaacgctgaccccgtgctgatcatcttcggctgcttttgcggctttatcctgatcggcctgatcctgtacatcagcctggccatcagaaagcgggtgcaagagacaaagttcggcaacgccttcaccgaagaggacagcgagctggtggtcaactatatcgccaagaagtccttctgcagacgggccatcgagctgacactgcacagtctgggagtgtccgaggaactgcagaacaagctggaagatgtggtcatcgaccggaacctgctgatcctgggcaagattctcggcgagggcgagtttggctctgtgatggaaggcaacctgaagcaagaggacggcacctctctgaaggtggccgtgaaaaccatgaagctggacaacagcagccagcgcgagatcgaagagtttctgtctgaggccgcctgtatgaaggatttctctcaccccaacgtgatccggctgctgggcgtgtgtatcgagatgtctagccagggcatccccaagcctatggtcatcctgcctttcatgaagtacggcgatctgcacacctacctgctgtactccagactggaaacaggccccaagcacatccctctgcagaccctgctgaagttcatggtggatatcgccctcggcatggaatacctgagcaaccggaacttcctgcaccgcgatctggccgccagaaattgcatgctgagggacgacatgaccgtgtgcgtggccgattttggcctgagcaagaagatctacagcggcgactactaccggcagggcagaattgccaagatgcccgtgaagtggatcgccatcgagagcctggccgacagagtgtacaccagcaagtctgacgtgtgggccttcggcgtgaccatgtgggagattgccaccagaggcatgaccccttatcctggcgtccagaaccacgagatgtacgattacctgctgcacggccacagactgaagcagccagaggattgcctggacgagctgtacgagatcatgtactcttgctggcggaccgatccactggacagacctacattctccgtgctgcggctgcagctggaaaaactgctggaaagcctgcctgacgtgcggaaccaggccgatgtgatctacgtgaacacccagctgctggaatccagcgaaggactggcccagggatctacactggctcctctggacctgaacatcgaccccgacagcattatcgccagctgcacaccaagagccgccatcagcgttgtgacagccgaggtgcacgatagcaagcctcacgaaggccggtacatcctgaatggcggaagcgaggaatgggaagatctgaccagcgctccttctgccgccgtgaccgccgagaaaaattctgtgctgcctggcgagcggctcgtgcgaaacggtgtctcttggagccacagctccatgctgcctctgggaagctctctgcctgatgagctgctgttcgccgacgattctagcgagggatccgaggtgttgatg

SEQ ID NO: 126 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 127 - protein, 31 aa, Homo sapiens
EGISLLAQNTSWLLLLLLSLSLLQATDFMSL

SEQ ID NO: 128 - protein, 15 aa, synthetic construct
GGGGSGGGGSGGGGS

SEQ ID NO: 129 - protein, 48 aa, synthetic construct
DNLLPSWAITLISVNGIFVICCLTYCFAPRCRERRRNERLRRESVRPV

SEQ ID NO: 130 - protein, 41 aa, Homo sapiens
IEVMYPPPYLDNEKSNGTIIHVKGKHLCPSPLFPGPSKPGS

SEQ ID NO: 131 - protein, 10 aa, synthetic construct
RPKPVELWRK

SEQ ID NO: 132 - protein, 233 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSEGISLLAQNTSWLLLLLLSLSLLQATDFMSL

SEQ ID NO: 133 - protein, 250 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNGGGGSGGGGSGGGGSDNLLPSWAITLISVNGIFVICCLTYCFAPRCRERRRNERLRRESVRPV

SEQ ID NO: 134 - protein, 259 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNIEVMYPPPYLDNEKSNGTIIHVKGKHLCPSPLFPGPSKPGSEGISLLAQNTSWLLLLLLSLSLLQATDFMSL

SEQ ID NO: 135 - protein, 260 aa, synthetic construct
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNRPKPVELWRKGGGGSGGGGSGGGGSDNLLPSWAITLISVNGIFVICCLTYCFAPRCRERRRNERLRRESVRPV

SEQ ID NO: 136 - protein, 776 aa, synthetic construct
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPMDYKDDDDK

SEQ ID NO: 137 - DNA, 498 bp, synthetic construct
  source 1..498: mol_type = other DNA
atgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaat

SEQ ID NO: 138 - DNA, 93 bp, synthetic construct
  source 1..93: mol_type = other DNA
gagggaattagcctgctggcccagaataccagctggctgctgctgcttctgctgagcctgtctctgctgcaagccaccgacttcatgagcctg

SEQ ID NO: 139 - DNA, 45 bp, synthetic construct
  source 1..45: mol_type = other DNA
ggcggcggaggatctggcggaggtggaagcggaggcggtggatct

SEQ ID NO: 140 - DNA, 63 bp, synthetic construct
  source 1..63: mol_type = other DNA
ctgctgcctagctgggccatcacactgatctccgtgaacggcatcttcgtgatctgctgtctg

SEQ ID NO: 141 - DNA, 123 bp, synthetic construct
  source 1..123: mol_type = other DNA
atcgaagtgatgtaccctccaccttacctggacaacgagaagtccaacggcaccatcatccacgtgaagggcaagcacctgtgtccttctccactgttccccggacctagcaagcctggctct

SEQ ID NO: 142 - DNA, 30 bp, synthetic construct
  source 1..30: mol_type = other DNA
agacccaagccagtggaactgtggcggaaa

SEQ ID NO: 143 - DNA, 702 bp, synthetic construct
  source 1..702: mol_type = other DNA
atgaccaacaagtgcctgctgcagatcgccctgctgctgtgctttagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaatggcggcggaggatctggcggaggtggaagcggaggcggtggatctgagggaatttctctgctggctcagaacaccagctggctgctgctgcttctgctgtctctgagtctgctgcaggccaccgacttcatgagcctgtaa

SEQ ID NO: 144 - DNA, 753 bp, synthetic construct
  source 1..753: mol_type = other DNA
atgaccaacaagtgcctgctgcagatcgccctgctgctgtgctttagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaatggcggcggaggatctggcggaggtggaagcggaggcggtggatctgataatctgctgccctcctgggccatcacactgatctccgtgaacggcatcttcgtgatctgctgtctgacctactgcttcgcccctcggtgcagagagcggagaagaaacgaacggctgcggagagaatctgtgcggcctgtgtaa

SEQ ID NO: 145 - DNA, 780 bp, synthetic construct
  source 1..780: mol_type = other DNA
atgaccaacaagtgcctgctgcagatcgccctgctgctgtgctttagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaacatcgaagtgatgtaccctccaccttacctggacaacgagaagtccaacggcaccatcatccacgtgaagggcaagcacctgtgtccttctccactgttccccggacctagcaagcctggctctgagggaattagcctgctggcccagaataccagctggctgctgctgcttctgctgagcctgtctctgctgcaagccaccgacttcatgagcctgtga

SEQ ID NO: 146 - DNA, 783 bp, synthetic construct
  source 1..783: mol_type = other DNA
atgaccaacaagtgcctgctgcagatcgccctgctgctgtgctttagcacaacagccctgagcatgagctacaacctgctgggcttcctgcagcggagcagcaacttccagtgccagaaactgctgtggcagctgaacggccggctggaatactgcctgaaggaccggatgaacttcgacatccccgaggaaatcaagcagctgcagcagttccagaaagaggacgccgctctgaccatctacgagatgctgcagaacatcttcgccatcttccggcaggacagcagctccacaggctggaacgagacaatcgtggaaaatctgctggccaacgtgtaccaccagatcaaccacctgaaaaccgtgctggaagagaagctggaaaaagaggacttcacccggggcaagctgatgagcagcctgcacctgaagcggtactacggcagaatcctgcactacctgaaggccaaagagtacagccactgcgcctggaccatcgtgcgcgtggaaatcctgcggaacttctacttcatcaaccggctgaccggctacctgagaaacagacccaagccagtggaactgtggcggaaaggtggcggaggatctggcggaggtggaagcggcggaggcggatctgataatctgctgcctagctgggccatcacactgatctccgtgaacggcatcttcgtgatctgctgtctgacctactgcttcgcccctcggtgcagagagcggagaagaaacgaacggctgcggagagaatctgtgcggcctgtgtaa

SEQ ID NO: 147 - DNA, 2331 bp, synthetic construct
  source 1..2331: mol_type = other DNA
atggcccagtggaatcagctgcagcagctcgacaccagatacctcgaacagctccaccagctgtactccgacagcttccctatggaactgcggcagtttctggccccttggatcgagtctcaggattgggcctacgccgccagcaaagagtctcacgccacactggtgttccacaacctgctgggcgagatcgaccagcagtacagccggtttctgcaagagtccaacgtgctgtaccagcacaacctgaggcggatcaagcagttcctgcagagcagatacctggaaaagcccatggaaatcgcccggatcgtggccagatgtctgtgggaagaaagcagactgctgcagaccgccgctacagctgctcaacaaggcggacaggccaaccatcctacagccgccgtggttacagagaagcagcagatgctggaacagcatctccaggacgtgcggaagagagtgcaggacctggaacagaaaatgaaggtggtggaaaacctgcaggacgacttcgacttcaactacaagaccctgaagtcccagggcgacatgcaggatctgaacggcaacaaccagagcgtgacccggcagaagatgcagcaactcgaacagatgctgacagccctggaccagatgaggcggagcattgtgtctgaactggccggactgctgagcgccatggaatacgtgcagaaaaccctgaccgacgaggaactggctgactggaaaagacggcagcagatcgcctgtatcggcggacctcctaacatctgcctggaccggctggaaaactggatcacaagcctggccgaaagccagctgcagacaagacagcagatcaagaagctggaagaactgcagcagaaggtgtcctacaagggcgaccccatcaccaagaacaaacaggtgctgtgggacagaaccttcagcctgtttcagcagctgatccagagcagcttcgtggtggaacggcagccctgcatgcctacacaccctcaaagacccctggtgctgaaaaccggcgtgcagttcaccgtgaagctgcggctgctggtcaagctgcaagagctgaactacaacctgaaagtgaaggtgctgttcgacaaggacgtgaacgagcggaacaccgtgaaaggcttccgcaagttcaacatcctgggcacccacaccaaagtgatgaacatggaagagagcaccaacggctccctggccgccgagtttagacacctccagctgaaagagcagaagaacgccggcaccaggaccaatgagggacctctgatcgtgacagaggaactgcacagcctgagcttcgaaacccagctgtgtcagcctggcctggtcatcgatctggaaaccacctctctgcccgtggtggtcatcagcaatgtgtcccagctgccttctggctgggccagcatcctgtggtacaacatgctggtggccgagcctcggaacctgtccttctttctgacccctccatgtgccagatgggcccagctgtctgaagtcctgagctggcagtttagcagcaccaccaagagaggcctgagcatcgagcagctgacaacactggccgagaagttgcttggccctggcgtgaactacagcggctgtcagattacctgggccaagttctgcaaagaaaacatggccggcaagggcttcagcttctgggtctggctggacaacatcatcgacctggtcaagaagtacattctggccctgtggaacgagggctacatcatgggcttcatctccaaagagagagagcgggccatcctgagcacaaagcctccaggcaccttcctgctgagattcagcgagagcagcaaagaaggcggcgtcacctttacctgggtcgagaaggatatcagcggcaagacccagatccagtccgtggaaccctacaccaaacaacagctgaacaacatgagcttcgccgagatcatcatggggtacaagatcatggacgccaccaatatcctggtgtccccactggtgtacctgtatcctgacatccccaaagaggaagccttcggcaagtactgcagacccgagagccaagagcaccctgaagccgatcctggaagcgccgctccttacctgaaaacaaagttcatctgcgtgacccctaccacctgtagcaacacaatcgacctgcctatgagccccagaacactggactccctgatgcagttcggcaacaatggcgaaggcgccgaaccatctgctggcggacagtttgagtccctgaccttcgacatggaactgaccagcgagtgcgccacctctcctatggactacaaggacgacgacgacaagtga

SEQ ID NO: 148 - RNA, 400 nt, synthetic construct
  source 1..400: mol_type = other RNA
  misc_difference 1..400: note = This sequence may encompass 10-400 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 149 - RNA, 200 nt, synthetic construct
  source 1..200: mol_type = other RNA
  misc_difference 1..200: note = This sequence may encompass 10-200 nucleotides
cccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccc

SEQ ID NO: 150 - protein, 581 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSG

SEQ ID NO: 151 - protein, 617 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSGGGGGSGGGGSGGGGSPTSFGYDKPHVLVDLLVDDSG

SEQ ID NO: 152 - protein, 511 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSG

SEQ ID NO: 153 - protein, 547 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGTTTPAPRPPTPAPTIASQPLSLRPEACRPAAGGAVHTRGLDFACDIYIWAPLAGTCGVLLLSLVITLYCRLKIQVRKAAITSYEKSDGVYTGLSTRNQETYETLKHEKPPQSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSGGGGGSGGGGSGGGGSPTSFGYDKPHVLVDLLVDDSG

SEQ ID NO: 154 - protein, 578 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSG

SEQ ID NO: 155 - protein, 614 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSGGGGGSGGGGSGGGGSPTSFGYDKPHVLVDLLVDDSG

SEQ ID NO: 156 - protein, 508 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRLKIQVRKAAITSYEKSDGVYTGLSTRNQETYETLKHEKPPQSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSG

SEQ ID NO: 157 - protein, 544 aa, synthetic construct
MALPVTALLLPLALLLHAARPDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGIEVMYPPPYLDNEKSNGTIIHVKGKHLCPSPLFPGPSKPFWVLVVVGGVLACYSLLVTVAFIIFWVRLKIQVRKAAITSYEKSDGVYTGLSTRNQETYETLKHEKPPQSPNRKNPLWPSVPDPAHSSLGSWVPTIMEEDAFQLPGLGTPPITKLTVLEEDEKKPVPWESHNSSETCGLPTLVQTPTSFGYDKPHVLVDLLVDDSGGGGGSGGGGSGGGGSPTSFGYDKPHVLVDLLVDDSGGGGGSGGGGSGGGGSPTSFGYDKPHVLVDLLVDDSG

SEQ ID NO: 158 - DNA, 1746 bp, synthetic construct
  source 1..1746: mol_type = other DNA
atggcccttcccgtgaccgccctgttgctgcctcttgcattgctcctgcacgccgctcgccccgacatccagatgacccagtctccctctagcctgtccgctagcgtgggagaccgggtgactatcacatgccgcgcgagccaagacgtgaatactgctgtggcttggtaccaacagaagccagggaaggcccccaaactcctgatctactctgcaagctttctttactccggtgtgccgtcaagattcagcggcagcagaagcggcaccgactttacactgaccatttctagcctccaaccggaggacttcgctacctactattgtcaacagcactacactacgccccctaccttcggtcagggcaccaaagtggagattaagcgtactggttcaacatccggctccgggaagcccggcagcggcgagggctccgaggtgcaactggtggagtctggtggcggactcgtgcagcctgggggctcccttcgcctctcatgcgcagcgagtggcttcaacatcaaagacacttatatccactgggtgcgtcaggcaccgggtaagggcctggagtgggtggctagaatctatcctaccaacggatacactcgctacgcagattccgttaaaggccggttcaccatctccgccgacacttccaagaacaccgcttatctccagatgaacagccttcgcgcggaagacaccgctgtgtactattgctcccgctggggtggcgatggcttctatgccatggacgtgtggggccaaggaaccctggtgaccgtgtcctctagcggtaccactaccccggccccaagaccaccgacccctgctccaaccatcgcttctcagcctctttcactgcggccggaagcttgtcgccccgctgccgggggcgctgtccatacaagaggcctcgattttgcctgtgacatctacatctgggcaccgctcgctggaacctgtggtgtgctgctcctgagcctcgtgatcaccctgtattgcagagtgaagttcagccgcagtgcagacgcccctgcctaccaacaggggcagaaccaactgtacaacgagctgaacctgggccgtagagaagagtacgatgtgctggataagaggcggggccgggatcctgaaatgggaggcaagccccggcgcaagaacccacaagaaggcctgtacaacgagctgcagaaggataagatggctgaggcatatagtgagattggaatgaaaggagagcgcagacgcggcaagggccacgacggcctgtaccagggactgagcacggcaacaaaagacacctacgacgctttgcacatgcaggccctgcccccgcgcagtcccaatcgcaaaaatcccttgtggccctccgtccctgatccggctcacagctccctcgggtcctgggtgcccaccatcatggaagaggatgctttccagctcccaggcctgggcactcccccaatcaccaagttgaccgtcttggaagaggacgagaaaaagcctgtgccctgggagtcccataactcctcagagacatgcggtctgccaaccctggtgcagaccccaacatccttcggctacgataaaccccacgtcctggtcgatctgctcgttgacgattccggcgggggcggaggtagcggtggcggtgggtccggcgggggcgggtcacctactagcttcggttacgataagccccatgtcctggtggacttgcttgtggatgactcaggctaa

SEQ ID NO: 159 - DNA, 1854 bp, synthetic construct
  source 1..1854: mol_type = other DNA
atggcgctgcctgtcaccgccctgctcctgccgttggcccttctgctccacgcagccagacctgatattcaaatgactcagtcacctagctccctctccgcttcagtgggtgaccgcgtgacaatcacttgccgcgccagccaggacgtgaacactgccgtggcctggtaccaacagaagccgggaaaggccccgaagctcctgatttattctgcatcctttctgtattcaggtgttccaagcagattttctggtagccgctccggaacagattttacactgacaatctccagcctgcagcccgaagattttgcgacttattactgtcaacagcactacaccacgcccccaacttttggccagggcactaaggtggagatcaagcgtacgggttccaccagtggttccggtaagcccggcagcggggagggtagcgaagtacagctggtggagtccggcgggggcctggtccagcctggaggctccctgagactgtcctgcgctgccagcggattcaacattaaggacacatacatccactgggtgcgccaggcacctgggaagggcctggagtgggtggcccggatctaccctactaacggatacacccgctacgccgattccgtgaaggggcggtttactatctccgctgatacgtcaaagaacaccgcttacctccagatgaactctctgagggcagaggataccgcagtgtattactgctctaggtggggtggggacgggttctacgctatggatgtatggggccagggcacactggtcactgtatctagctctggaacgacaactcccgcaccccgccctccaacccctgctcccacaatcgcctctcagccactgagtttgaggcctgaggcctgtcgcccagccgctggaggtgctgtccatactcgtggactggactttgcctgtgacatctatatctgggcccctctggctggcacatgtggcgtgttgctgctttctctcgttatcaccctgtactgccgggtgaaattcagccggtccgctgacgcaccagcttatcaacagggacaaaaccagctgtacaacgaattgaatctggggcggcgtgaggaatacgacgtcctggacaaacgtcgcgggcgcgatcccgagatgggtggcaagcctcgcaggaagaacccgcaggagggtctttacaacgagctgcagaaggacaagatggctgaggcatacagcgagatcggtatgaagggggagaggcgtcgcggtaagggccatgacggcttgtaccagggactctccactgccacaaaagatacttacgacgctctgcacatgcaggctctgcctccacgttcccccaataggaagaatcccctttggccatccgtgcctgaccctgcccacagttccctcggctcttgggtgcccaccatcatggaagaggatgcctttcagctgcccggtctgggcacgccacccatcactaagctgaccgtgctggaagaggacgagaaaaagcctgtgccttgggaaagccacaattctagcgagacctgtggcctcccgactttggtgcagactccaactagttttgggtacgataagccccacgtgttggtagacttgctggtggatgactccggcgggggtggcggatccggcggaggcgggagtggcggtggcggttcccccacttctttcggatatgacaagccgcacgtgctggtggatttgctggtggatgactccggcgggggcggagggagcggtggcggaggctccgggggcgggggctctcccacatcttttggctacgacaaacctcatgttctggtcgacttgctggttgatgactccggataa

SEQ ID NO: 160 - DNA, 1536 bp, synthetic construct
  source 1..1536: mol_type = other DNA
atggccctgccggttaccgcgctgcttctgcccctggcactgttgctgcacgcggcccggcctgacatccagatgacacagtccccatcttccctgtctgcgagcgtgggagaccgtgtgaccattacctgcagggcttcccaggatgttaacactgctgtggcttggtaccagcaaaagcctggtaaggctcccaagctcctgatctactccgcatctttcctctactcaggcgtgcctagcaggtttagtggtagccggtctggaacggacttcacccttacaatttcttccctgcagccagaagacttcgccacctattactgtcaacagcactataccacacctcccacgtttggacaggggaccaaagtggagatcaagagaactggttccacttcaggtagcgggaagccaggatcaggtgaaggctccgaggtccagttggtcgagagcggtggaggtctggtgcagccaggaggctcactgagattgtcttgtgctgccagcggcttcaatatcaaggatacctacatccactgggtacgccaggcccccggcaagggactggagtgggtggctcgcatctaccctacaaacggctacaccaggtacgcggatagtgtcaagggccggtttaccattagcgcggacaccagcaagaacaccgcttaccttcagatgaactccctgcgggcggaagacaccgccgtgtattactgttctcgctggggcggtgacggattctacgccatggacgtttggggtcagggcaccctggttacggtctccagttccggcacaaccactcccgcccctagaccgccaacacctgctcctacgatcgcctcacagcccttgtcccttagaccagaggcctgtcgtccggcggccggaggcgccgtgcacaccagagggctcgattttgcttgcgacatctacatctgggctccactggcgggaacctgcggggtgcttttgctgagcctggttatcaccctgtactgtcgcctgaaaatccaggtgcgcaaagctgccattacctcttacgaaaagagtgatggcgtgtataccggcctctccacaagaaaccaggaaacttatgaaaccctcaagcacgagaagcccccacagtcccctaaccgtaagaacccattgtggccttctgtgcctgatcccgctcatagtagcctcggatcctgggtcccgaccatcatggaagaggacgcctttcagctgcctggcctcgggacccctccaatcacaaagctgacagttctggaagaggacgagaaaaagccagtgccctgggaatcccacaattctagcgagacctgtggcctgcccaccctggtccagaccccaacttctttcggatatgataagccgcacgtgctggttgatcttctggtagatgactccggaggtggcggtgggtctggcgggggtggatccggcgggggcgggagcccaacttctttcggttatgataagcctcatgtgcttgtagatctcctggttgatgactccggctga

SEQ ID NO: 161 - DNA, 1644 bp, synthetic construct
  source 1..1644: mol_type = other DNA
atggccctgccagtcactgctctgttgctgccactggccttgctcctgcacgctgcaaggccagatattcagatgacacagagcccatcctctctgtctgccagcgtcggtgatagagtgaccatcacgtgccgggcttcccaagacgtcaacacagcggttgcttggtatcaacagaaaccgggcaaggcacccaagctcctgatctattccgccagtttcctgtactccggggtgccgtcccggttcagtggttctcggtctggcacggattttactctgacgatctccagcctccagcctgaggacttcgccacctattactgccaacagcactacacaactcccccaacctttggtcagggcaccaaggtggagatcaagcgtaccggctccactagcggcagtgggaaacctggctcaggggaagggtctgaagtgcagctggtggagtctggaggcggtctcgtacagccaggtggcagcctgcgcctgtcctgcgctgcctccggattcaacatcaaagacacctacatccattgggttcgccaagcccccggcaagggccttgagtgggtagccaggatttaccccaccaatggctacacccgctacgccgactccgttaagggccgtttcacgatctctgcagacacctctaagaataccgcctatctgcagatgaattctctgcgcgctgaggacacagcggtgtactattgcagcagatggggaggtgacggtttttatgccatggacgtctggggccagggcactctggttacggtgagttcttccgggactacaaccccggcacccaggccccctactcccgctcccaccatcgcctcccagccgctgtctctgcgtcccgaggcctgtcgtccggcggcagggggcgccgtgcacaccagaggcctggatttcgcttgtgacatctacatctgggctccactcgcagggacgtgtggcgtcctgctcctgagcctggtcatcaccctctattgtcgcctgaagatccaggtgcgcaaggctgccatcaccagctatgaaaagtccgacggagtatacacaggactttccacacgcaaccaggaaacttacgagactttgaagcatgagaagccgccccagagtcccaacagaaagaaccccctttggccgagcgtcccggatcccgcgcattcttccctgggatcctgggtgcccaccattatggaagaggatgcattccagctccctggcctcggcacaccccctattaccaaactcaccgtgctggaagaggatgagaagaaacctgtgccatgggaaagccacaacagctccgagacctgtggcctgcctaccctggttcagactcctacatcctttgggtatgataagccccacgtgctggttgatcttctggttgatgacagcggtggcggaggtggcagcgggggcggtggaagcggtggaggcggatccccaacatctttcggctacgacaagccccacgtgctggtagacttgctggtcgatgactccggtggaggcggaggctctggcggtggaggctctggaggcggtggctcccctaccagctttgggtacgataaaccgcatgttctggtggatttgctggtcgatgactctggctaa

SEQ ID NO: 162 - DNA, 1737 bp, synthetic construct
  source 1..1737: mol_type = other DNA
atggccctgcccgtgaccgccttgctcctgcccttggcccttctcctgcatgcggccagacccgatatccagatgactcagtccccttccagcctctccgcctctgtgggtgatagagtcaccatcacttgtcgggcttcacaagacgtgaacaccgccgtggcctggtatcaacagaagcccggcaaagcaccaaaactcctgatttactccgccagcttcctgtacagcggagtgccctcccgcttcagcgggagtcggagcggcacagacttcaccctgaccatttccagtctgcagccagaggacttcgctacatattactgtcaacagcactacaccacgccacctacctttgggcaggggacgaaggtagaaatcaaacgcaccggctccacatccggaagcggaaagcctggcagcggcgagggctcagaggtccagctggtggagtccgggggtggcctggtgcagcccggcggttccctgcgcctctcttgcgcggcttccggtttcaacatcaaagacacctacatccactgggttcgccaggccccaggaaagggcctcgagtgggtggctcgcatctatcctaccaatgggtacacacgctacgccgactccgtgaagggccgctttactatcagcgcagacacaagcaagaacacagcctacctgcaaatgaactcactccgcgccgaggacaccgcggtgtattactgctcccgctggggaggtgacggcttctacgccatggacgtgtgggggcagggaacactcgtcaccgtgtctagctccgggatcgaagtaatgtatcccccgccttacctggacaacgagaaatctaacggcacgatcattcatgtcaagggaaagcacctgtgcccatcccctctgttccctggcccgtccaagcccttttgggttctggtggttgtcggaggcgtgctggcatgctactcactcctggtcaccgttgcctttattatcttctgggtgcgcgtgaagttctccagatccgctgatgccccagcatatcaacagggacagaatcagctgtataatgaactgaacctgggcaggcgcgaagagtacgatgtgctcgataaacgcaggggccgcgatccagaaatgggaggcaagcccagacgcaaaaacccgcaggaaggcctgtataacgaactccagaaggacaagatggccgaggcctatagcgaaatcgggatgaagggcgaacgtagacgcggcaagggccacgacgggctctaccaaggtctgtccacagcgaccaaagatacatacgacgcccttcacatgcaagccctgccccctcgttcccctaaccgtaagaacccattgtggccctccgtgccggacccagctcacagctccctgggctcatgggtgcccaccatcatggaagaggacgcattccagctgcccgggctcggcacccctcccatcactaagcttacagtcctcgaagaggatgagaaaaagcccgtgccctgggaatcccacaacagctccgagacctgtggtctccccacccttgtgcagacacctacttccttcgggtatgataagccacatgttcttgtggacctcttggttgacgatagcgggggtgggggcggttccggcgggggtggatccggaggtggcggatcccccacaagcttcgggtacgacaagccccacgttttggtggaccttctggtggatgactccggttaa

SEQ ID NO: 163 - DNA, 1845 bp, synthetic construct
  source 1..1845: mol_type = other DNA
atggcgctgcccgtgaccgcgctccttttgcctcttgcactgctcctgcacgctgcacgtccggacatccagatgactcaatctcctagcagtttgtctgcctctgtgggcgaccgggttaccatcacatgtcgggccagccaggacgtgaacaccgccgtggcatggtatcaacagaagcccggcaaggctccaaagctcctgatttacagtgcctctttcctgtattccggtgtaccctctcgtttctccggatctcgcagcgggacggattttaccctgaccatcagctccctgcagccggaagattttgctacatactattgccaacagcactacaccactcccccgactttcggccagggcacaaaggtggagattaaacgtaccggctcaacctctgggtctggcaaacccgggtctggcgagggatcagaggtgcagctggtagaatcaggcggtggcctggtgcagcctggcgggagcctccgtctgtcctgtgcggccagcggctttaatatcaaggacacctatatccattgggtcagacaagcacctggcaaggggctggaatgggttgctcgtatctacccaactaatggttatacaaggtatgccgactctgtcaagggccgttttacaatctccgccgacacctctaaaaataccgcttatctccagatgaacagtctcagagctgaggacacagccgtgtattactgcagtagatggggcggggatggcttctatgcgatggatgtctgggggcagggcacacttgttaccgtctcatcttccggcatcgaggtgatgtatcccccaccttaccttgataatgagaaatcaaacgggaccattatccatgtgaaaggtaagcatctgtgccccagtcccctcttccctggacctagcaagcccttctgggtgctggtagtcgttgggggcgtgctggcctgctactccctgttggtgaccgtggcctttattatcttctgggtgcgtgtcaagttttccaggtcagccgacgcccccgcctaccagcaaggtcagaaccaactttataatgagttgaatctgggacgccgtgaagagtatgacgtcctcgacaagcggcgcgggcgcgatcccgagatgggtggcaagcctcgtcgcaagaaccctcaagagggcttgtataacgagctgcagaaggacaagatggctgaagcttactccgagatcggaatgaagggcgagcgtcgccgtggaaagggacacgacggtctgtaccaaggtctctccaccgccactaaggacacctacgacgctctgcacatgcaagccttgccgccccgctcacccaaccgcaaaaaccctctctggcccagcgtgccagatcctgcacactcttccctgggtagctgggtgcctacaattatggaagaggacgcctttcagctcccgggtctgggaactcctcccatcaccaagctgaccgtgctcgaagaggacgagaagaaaccggtaccctgggagtctcacaactcttccgagacctgtggcctgcctaccctcgtccaaactcccacttcatttggttatgacaagcctcatgtactggtggacttgctggtggacgattcaggtggcgggggtggctcaggcggtggaggctccgggggcgggggctctcctacgtcttttggctacgacaagcctcacgttttggtcgatttgctcgttgatgactctggcggaggcggtggctccggcggaggtggaagcggcgggggagggagccccacttccttcggctacgataagcctcacgttctcgtcgatcttctggtggacgattcaggataa

SEQ ID NO: 164 - DNA, 1527 bp, synthetic construct
  source 1..1527: mol_type = other DNA
atggctctgccagtcactgctctgttgctgccgctggccctgctcctgcacgccgctcgccctgacatccagatgacccagtcaccatcctcactgagcgcctctgtcggagatagggtgaccattacttgccgcgcatctcaggacgtgaacacagctgtcgcctggtaccaacagaagcccggaaaggcccccaagcttctgatttactctgcgagcttcctgtatagcggtgtaccctctagattcagtggaagccggtcaggtacagacttcaccctgaccatttcatccctccagccggaagatttcgcaacatattactgccaacagcactacactaccccgccaacgtttggtcagggcacaaaggtcgagatcaaacggacaggatccacaagtggttctggcaagcctggcagtggtgagggcagcgaagttcaattggtggaatctggcggaggcctggtccagcccgggggtagcctgcgcctgtcatgcgccgcgtccggcttcaacattaaggacacctacattcattgggtgcgccaggctcccgggaagggtctggagtgggtcgcccgcatctaccccactaatggctacacccgttacgctgactccgtgaagggccgtttcaccatttccgccgacacctccaagaataccgcatatctccagatgaactccctgcgcgctgaggacacagcagtgtattactgttctcgctggggtggagacggtttctatgccatggacgtttggggccagggcacactggttaccgtgagctccagcggcatcgaagtaatgtaccctccgccttatctggataacgagaaatccaatggaaccattatccacgtcaaaggcaaacacctgtgtccgtcccctttgttccctggaccctctaagcccttctgggtgctcgtcgtggtaggcggggtgctggcctgttactccctcctggtaacagtagctttcattatcttctgggtgcgcctgaagattcaggtccgtaaggcagctatcacctcctatgagaagagcgacggtgtctataccggcctgagcacccggaaccaggagacttatgagaccctcaagcacgaaaagcctccacagagtcccaatcggaagaatcccctttggccttctgtgcctgatccagcccactcctctcttggtagctgggtgcctacaattatggaagaggacgcgtttcagctgcccggcctgggcaccccacccatcacaaagctgaccgtgctcgaggaagacgagaagaaacccgtgccatgggagagtcataactcaagcgagacctgcgggttgcccaccctggtccagactcccaccagctttggatatgacaagcctcatgtactggtggatttgctggtggacgattccgggggcggaggcggttcaggcggaggtggatccggtggaggtggctctcccacctcattcggttatgacaagccccacgtgttggtggacctccttgtggacgattctggttaa

SEQ ID NO: 165 - DNA, 1635 bp, synthetic construct
  source 1..1635: mol_type = other DNA
atggccctgccggtcactgcactccttctgccgctggcactgctcctgcacgctgcacgtcccgacatccagatgacccagtcaccgagctctctctccgcttctgtgggtgaccgtgtgaccatcacctgccgtgcctctcaggacgttaacaccgctgtggcctggtaccagcaaaaaccaggcaaagcccccaaactccttatttacagtgcttcattcctgtactccggcgtccctagcagatttagcggctcccgcagcggaacagattttacactcaccatctcctcattgcagcccgaggattttgccacctattactgccaacagcattacacaacccctcccaccttcgggcagggaactaaggtcgagatcaagcgcacaggctctacttccgggtccgggaagccaggttccggagagggctctgaggtacaactggttgaaagtgggggcggtctcgttcagccgggaggctccctgcgcctgtcctgcgccgcgtctggttttaacattaaagatacctatattcactgggtgcgtcaagctccaggcaagggactggagtgggtggcgcgcatctaccccactaacggttacacccgctacgccgacagcgtcaaggggcgttttaccatctctgccgacacctccaagaacacagcctatcttcaaatgaacagcttgcgcgccgaggacaccgcggtgtattactgctctcgctggggcggagacggtttctacgccatggacgtgtggggccagggcacgctggtgactgtcagctcttcaggtatcgaggtcatgtacccacccccatatttggacaatgagaagtctaatggcaccattatccacgtgaaaggtaagcatctctgtcccagtccgctgtttcccggtccaagtaagcctttttgggtcctcgtcgtggtcgggggagtcttggcctgctactctttgctggtgaccgtcgcttttattatcttctgggtgcgcctcaaaatccaggttcggaaggcggccatcacaagctacgagaagagcgacggcgtttataccggcctgtccacccgtaatcaggaaacatatgaaaccctgaagcacgaaaagccaccccagagtcccaaccgtaagaacccactctggccttctgtccccgatcccgcccattcatccttgggtagttgggtgccaactatcatggaagaggatgcattccagctccctggcctgggcactcctcccatcaccaaactgacggttctggaggaagacgaaaagaaaccggtgccgtgggagagccacaattcctcagagacatgtggtctgccaacactggtgcaaacgcctaccagcttcgggtatgacaagccgcacgtcctcgtggacctcctggtggatgactccggcggtgggggtgggtctggcggtgggggctccggcggaggcgggtctccaacttcctttggctacgataaaccacacgtccttgtggaccttctggtggatgacagcgggggcgggggcggtagcgggggcggtggatcaggcggaggcggtagccctacctctttcggttatgacaagccccatgtattggtagaccttctggtggatgacagcggatag

SEQ ID NO: 166 - protein, 4 aa, unidentified
DEAD